(19) United States
(12) Patent Application Publication
(10) Pub. No.: US 2026/0232812 A1
(43) Pub. Date: Aug. 13, 2026

(54) BIFUNCTIONAL MOLECULES FOR TARGETED PROTEIN DEGRADATION

(71) Applicant: Amphista Therapeutics Limited, Cambridge (GB)

(72) Inventors: Andrea TESTA, Cambridge (GB); Callum MACGREGOR, Cambridge (GB); David MCGARRY, Cambridge (GB); Gregor MEIER, Cambridge (GB)

(21) Appl. No.: 18/874,153

(22) PCT Filed: Jun. 16, 2023
(86) PCT No.: PCT/GB2023/051592

§ 371 Date: Dec. 12, 2024

(30) Foreign Application Priority Data
Jun. 16, 2022 (GB) 2208866.0
Dec. 20, 2022 (GB) 22192702.2

(51) Int. Cl. A61K 47/54; A61P 35/00; G01N 33/50

(52) U.S. Cl. CPC A61K 47/545; A61P 35/00; G01N 33/502

(57) ABSTRACT

The present disclosure relates to a novel class of bifunctional molecules that are useful in a targeted or selective degradation of a protein.

### FIELD

The present disclosure relates to a novel class of bifunctional molecules that are useful in a targeted or selective degradation of a protein.

### BACKGROUND

Targeted Protein Degradation (TPD) is a therapeutic modality, which relies on the use of synthetic molecules to repurpose intracellular protein degradation machinery to induce degradation of specific disease-causing proteins. TPD approaches offer a number of advantages over other drug modalities (e.g. small molecule inhibitors, antibodies & protein-based agents, antisense oligonucleotides and related knockdown approaches) including: potentiated pharmacology due to catalytic protein removal from within cells; ability to inhibit multiple functions of a specific drug target including e.g. scaffolding function through target knockdown; opportunity for systemic dosing with good biodistribution; potent in vivo efficacy due to catalytic potency and prolonged duration of action limited only by de novo protein resynthesis; and facile chemical synthesis and formulation using application of small molecule processes.

The majority of physiologic post-translational regulation of intracellular protein levels as well as removal of damaged, misfolded, or excess proteins is mediated by the ubiquitin-proteasome system (UPS). The UPS relies on a complex cascade of protein-protein interactions that enables the polypeptide ubiquitin to be covalently attached to the protein intended for removal. The ubiquitin on the protein then acting as a marker or tag to the proteasome which then degrades and removes the protein from the cell.

The UPS can be repurposed to degrade specific proteins using bifunctional chemical molecules, commonly referred to as bifunctional degraders, as therapeutic agents. These molecules act by inducing the proximity of desired substrates with UPS proteins to initiate a cascade of events which ultimately leads to degradation, and removal of the protein from the cell by the proteasome.

Proteolysis targeting chimeras (PROTACs) constitute one such class of bifunctional degraders, which induce proximity of target proteins to the UPS by recruitment of specific ubiquitin E3 ligases. PROTACs are composed of two ligands joined by a linker-one ligand to engage a desired target protein and another ligand to recruit a ubiquitin E3 ligase.

The ubiquitin E3 ligases used most frequently in PROTACs are von Hippel-Lindau (VHL) and Cereblon (CRBN). PROTACs recruiting VHL are typically based on hydroxyproline-containing ligands, whereas PROTACs recruiting CRBN are typically characterised by the presence of a glutarimide moiety, such as thalidomide, pomalidomide and lenalidomide or close analogues to act as the warhead. Other ligases including mdm2 and the IAP family have also shown utility in PROTAC design.

However, these approaches suffer from a range of limitations, which restrict their utility to treat a wide range of diseases. For example, limitations of current PROTAC approaches include: inability to efficiently degrade some targets; poor activity of PROTACs in many specific cells due to low and variable expression of E3 ligases and other proteins required for efficient degradation; chemical properties which make it more difficult to prepare degraders with suitable drug-like properties including good drug metabolism & pharmacokinetic profiles; and high susceptibility to induced resistance mechanisms in tumours.

Because of these limitations, there remains a need to identify novel degrading mechanisms and warheads able to deliver new bifunctional degrader molecules, which show efficient degradation across a range of targets and cellular systems and/or with improved profiles suitable for drug development.

Further bifunctional degrader molecules have been described in WO 2019/238886, WO 2019/238817, WO 2019/238816, and WO 2022/129925.

### SUMMARY

The present disclosure is based on the identification of a novel class of bifunctional molecules that are useful in a targeted and/or selective degradation of a desired protein, e.g. a “target protein”. In particular, the present disclosure provides bifunctional molecules, which facilitate proteasomal degradation of selected target protein(s) using a novel class of warhead.

The bifunctional molecules described herein comprise a general structure of:

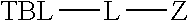

wherein TBL is a target protein binding ligand and L is a linker. The moiety “Z” (a “warhead”) modulates, facilitates and/or promotes proteasomal degradation of the target protein and may, in some cases, be referred to as a modulator, facilitator and/or promoter of proteasomal degradation. For example, in use, the TBL moiety of the bifunctional molecule binds to a target protein. The moiety Z (which is joined or otherwise connected to the TBL via the linker) then modulates, facilitates and/or promotes the degradation of this target protein, e.g. by acting to bring the target protein into proximity with a proteasome and/or by otherwise causing the target protein to be marked for proteasomal degradation within a cell.

Thus, the bifunctional molecules described in the present disclosure may be considered to comprise: a target protein binding ligand (TBL) (i.e. a ligand capable of binding (e.g. specifically binding) to a target protein; a warhead or degradation tag (Z) (e.g. moiety Z which acts to modulate, facilitate and/or promote the degradation of this target protein) and a linker (e.g. a chemical linker) which conjugates, joins or connects TBL and Z.

The bifunctional molecules described in the present disclosure have been shown to be effective degraders against a wide range of target proteins. Without being bound by theory, it is hypothesised that the Z moiety of the bifunctional molecules described herein does not bind to the ubiquitin E3 ligases typically relied on in the classical PROTAC approaches discussed above (such as CRBN and VHL). Accordingly, the bifunctional molecules described herein are believed to modulate, facilitate and/or promote proteasomal degradation via an alternative mechanism. Thus, the present class of bifunctional molecules may be useful against a wider range of diseases (including those that are resistant to many PROTAC degraders).

According to a first aspect of the disclosure, there is provided a bifunctional molecule comprising the general formula:

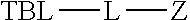

wherein TBL is a target protein binding ligand;
L is a linker; and
Z comprises a structure according to formula (ZI):

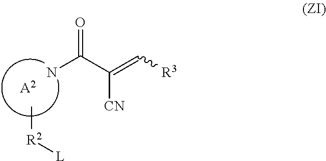

wherein:
ring A² is an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S;
R² is absent or is selected from aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
wherein R^{y} is optionally substituted C₁₋₆alkyl or H;
R³ is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S; and
L shows the point of attachment of the linker;
and further wherein Z is not:

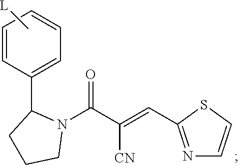

or a pharmaceutically acceptable salt thereof.

For the avoidance of doubt, in embodiments when ring A² is an optionally substituted 4- to 7-membered monocyclic N-heterocycloalkyl, ring A² is not fused to an aromatic ring. In embodiments when ring A² is an optionally substituted 7- to 12-membered bicyclic N-heterocycloalkyl, or an optionally substituted 8- to 18-membered tricyclic N-heterocycloalkyl, the term ‘heterocycloalkyl’ is intended to take its standard meaning in the art to exclude any of the two or three rings within these systems being aromatic. In other words, Z does not comprise, for example, a tetrahydroquinoline or other fused heterocycloalkyl/aryl or fused heterocycloalkyl/heteroaryl system.

As shown in formula (ZI) above, the linker may be appended to moiety Z via the R² group. In such examples, the linker may be attached to moiety Z by way of a covalent bond between an atom on the linker and an atom contained in the ring system of the aryl, substituted aryl, heteroaryl, substituted heteroaryl, heterocycloalkyl or substituted heterocycloalkyl of the R² group. Alternatively, the linker may be attached to moiety Z by way of a covalent bond to the nitrogen atom of NR^{y} or the benzylic carbon atom of the —CH(aryl)- or —CH(substituted aryl)-, for example by way of a covalent bond to the benzylic carbon atom of the —CH(aryl)- or —CH(substituted aryl)-.

As described above, in some examples R² may be absent. In such examples, the linker may be appended to moiety Z by way of a covalent bond between an atom on the linker and an atom contained in the heterocyclic ring (e.g. ring A²).

In all of the examples, the linker may be attached at any suitable position e.g. provided it has the correct valency and/or is chemically suitable. For example, the linker may be bonded at any position on the aryl, substituted aryl, heteroaryl, substituted heteroaryl, heterocycloalkyl, substituted heterocycloalkyl, NR^{y}, —CH(aryl)- or —CH(substituted aryl)- of the R² group or may replace a hydrogen atom at any position on the heterocyclic ring shown, for example, in formula (ZI).

As described above, ring A² is an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S, such as N and O.

When ring A² is bicyclic or tricyclic, and unless otherwise stated, it may comprise rings that are joined by a bond, rings that are fused, a bridged ring and/or rings that are joined at a spiro centre.

When ring A² is bicyclic, it may be a bridged bicyclic ring (i.e. it may comprise two rings that share three or more atoms) or it may be a spirocyclic bicyclic ring (i.e. it may comprise two rings that share one atom, e.g. the two rings may be joined at a spiro centre).

When ring A² is a bridged bicyclic ring, it may be an optionally substituted 7- to 12-membered bridged bicyclic N-heterocycloalkyl optionally containing one or two additional ring heteroatoms selected from N, O and S. In some examples, ring A² is a 7- or 8-membered bridged bicyclic N-heterocycloalkyl optionally containing one or two additional ring heteroatoms selected from N, O and S. In some examples, ring A² is a 7- or 8-membered bridged bicyclic N-heterocycloalkyl optionally containing one additional ring atom selected from N.

When ring A² is a spirocyclic bicyclic ring, it may be an optionally substituted 7- to 12-membered spirocyclic bicyclic N-heterocycloalkyl optionally containing one or two additional ring heteroatoms selected from N, O and S. In some examples, ring A² is a 7- to 12-membered spirocyclic bicyclic N-heterocycloalkyl optionally containing one or two additional ring heteroatoms selected from N, O and S. In some cases, ring A² is bicyclic and comprises a first 5- to 7-membered ring and a second 3- to 7-membered ring. For example, ring A² may be a spirocyclic bicyclic N-heterocycloalkyl comprising a first 5- or 6-membered ring and a second 3- to 6-membered ring, and optionally containing one or two additional ring heteroatoms selected from N, O and S. In some examples, ring A² may be a spirocyclic bicyclic N-heterocycloalkyl comprising a first 5- or 6-membered ring and a second 3- to 6-membered ring, and optionally containing one additional ring heteroatoms selected from N.

In some embodiments, Z comprises a structure according to formula (ZIa):

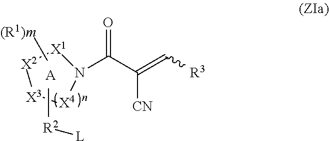

wherein:
R¹ is absent (i.e. when m is 0) or is selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R¹ groups combine to form an optionally substituted C₁₋₃ bridge, optionally substituted C₃₋₅cycloalkyl or optionally substituted 5- to 7-membered heterocycloalkyl (e.g. 5- to 7-membered N-heterocycloalkyl), optionally wherein the C₃₋₅cycloalkyl or the 5- to 7-membered heterocycloalkyl are joined to ring A at a spiro centre;
R² is absent or is selected from aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
wherein R^{y} is optionally substituted C₁₋₆alkyl or H;
R³ is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S;
X¹ is CH₂;
X², X³ and X⁴ are each independently CH₂, O or NR^{x};
R^{x} is H or C₁ to C₆ alkyl, or wherein one R¹ group and one R^{x} group combine to form an optionally substituted C₁₋₃ bridge;
n is 0, 1, 2, or 3;
m is 0, 1, 2, 3 or 4; and
L shows the point of attachment of the linker.

For the avoidance of doubt, in embodiments when ring A is not an aromatic ring, and is not fused to an aromatic ring. In other words, Z does not comprise, for example, a tetrahydroquinoline or other fused heterocycloalkyl/aryl or fused heterocycloalkyl/heteroaryl system.

In some examples, where n is 1, 2 or 3 (i.e. when 1, 2 or 3 X⁴ groups are present), an X⁴ group adjacent to (or directly bonded to) the N of the heterocyclic ring shown in formula (ZIa) is CH₂.

In some examples, Z comprises a structure according to formula (ZIb):

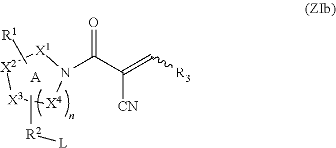

wherein:
R¹ is absent (i.e. when m is 0) or is selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R¹ groups combine to form an optionally substituted C₁₋₃ bridge, optionally substituted C₃₋₅cycloalkyl or optionally substituted 5- to 7-membered heterocycloalkyl (e.g. a 5- to 7-membered N-heterocycloalkyl), optionally wherein the C₃₋₅cycloalkyl or the 5- to 7-membered heterocycloalkyl are joined to ring A at a spiro centre;
R² is absent or is selected from aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
wherein R^{y} is optionally substituted C₁₋₆alkyl or H;
R³ is selected from C₁-C₆ alkyl, cycloalkyl, substituted cycloalkyl, alkylcycloalkyl, substituted alkylcycloalkyl, heterocycloalkyl, substituted heterocycloalkyl, alkyl heterocycloalkyl, substituted alkylheterocycloalkyl, aryl, substituted aryl, alkyl aryl, substituted alkylaryl, heteroaryl, substituted heteroaryl, alkyl heteroaryl, substituted alkylheteroaryl, optionally wherein the C₁-C₆ alkyl is substituted with one or more heteroatoms selected from halo, N, O and S;
X¹ and X⁴ are each CH₂;
X² and X³ are each independently CH₂, O or NR^{x}; with the proviso that none or only 1 of X² and X³ is O;
R^{x} is H or C₁ to C₆ alkyl; or wherein one R¹ group and one R^{x} group combine to form an optionally substituted C₁₋₃ bridge;
n is 0, 1, 2 or 3;
m is 0, 1, 2, 3 or 4; and
L shows the point of attachment of the linker.

In some examples, Z comprises a structure according to formula (ZIb′):

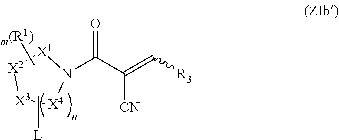

wherein:
R¹, R³, X¹, X², X³, X⁴, n, m and L are as defined above in respect of formula (ZIa) and (ZIb).

In some examples, Z comprises a structure according to formula (ZIb″):

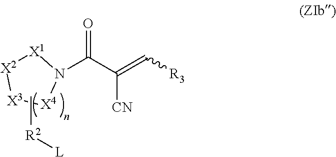

wherein:
R², R³, X¹, X², X³, X⁴, n and L are as defined above in respect of formula (ZIa) and (ZIb).

As stated above, in some embodiments of formulae (ZIa), (ZIb), (ZIb′), and (ZIb″) (and other formulae as described herein), an optionally substituted C₁₋₃ bridge may be formed by two R¹ groups or, in some cases, by one R¹ group and one R^{x} group. The C₁₋₃ bridge may be a C₁-C₃ alkylene bridging group, such as methylene, ethylene or propylene. In some examples, the C₁-C₃ bridge may be methylene or ethylene. Where the C₁₋₃ bridge is substituted, it may comprise from one to three (e.g. one or two) substituents (selected from any suitable substituent as described herein). For example, the C₁ to C₃ alkylene bridging group may be optionally substituted with one or two substituents each independently selected from the group consisting of halo, C₁ to C₃ alkyl, C₁ to C₃ haloalkyl and C₁ to C₃ alkoxy.

In further embodiments, Z may comprise a structure according to formula (I):

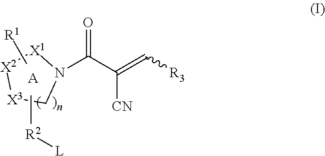

wherein R¹ is absent or is selected from aryl, substituted aryl, heteroaryl, substituted heteroaryl, cycloalkyl, C₁ to C₆ alkyl and substituted C₁ to C₆ alkyl;
R² is absent or is selected from aryl, substituted aryl, heteroaryl, substituted heteroaryl, heterocycloalkyl, substituted heterocycloalkyl, —NR^{y}, —CH(aryl)-, —CH(substituted aryl)-, —CH(heteroaryl)- and —CH(substituted heteroaryl)-;
wherein R^{y} is H or C₁ to C₆ alkyl;
R³ is selected from C₁ to C₆ alkyl, substituted C₁ to C₆ alkyl, aryl, substituted aryl, heteroaryl, and substituted heteroaryl;
X¹ is CH₂;
X² and X³ are each independently CH₂, or a heteroatom selected from O and NR^{x}, wherein R^{x} is H or C₁ to C₆ alkyl;
n is 0, 1, 2, or 3; and
L shows the point of attachment of the linker;
and further wherein Z is not:

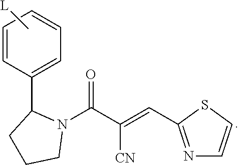

In alternative examples of formula (I), the list of options for R³ given above, may be replaced with C₁ to C₆ alkyl, cycloalkyl, substituted cycloalkyl, alkylcycloalkyl, substituted alkylcycloalkyl, alkyl heterocycloalkyl, alkylcycloalkyl, substituted heterocycloalkyl, substituted heterocycloalkyl, aryl, substituted aryl, heteroaryl, substituted heteroaryl, optionally wherein the C₁ to C₆ alkyl is substituted with one or more heteroatoms selected from halo, N, O and S. In some embodiments, R² may be absent or selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl)-.

In some examples, at least one of R¹ or R² is present.

For example, where R¹ is absent, R² may be present and selected from aryl, substituted aryl, heteroaryl, substituted heteroaryl, heterocycloalkyl, substituted heterocycloalkyl, —NR^{y}, —CH(aryl)-, —CH(substituted aryl)-, —CH(heteroaryl)- and —CH(substituted heteroaryl)-. For example, where R¹ is absent, R² may be present and selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl)-.

By way of further example, where R² is absent, R¹ may be present and selected from aryl, substituted aryl, heteroaryl, substituted heteroaryl, cycloalkyl, substituted cycloalkyl, C₁ to C₆ alkyl and substituted C₁ to C₆ alkyl. By way of even further example, where R² is absent, at least one R¹ may be selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R¹ groups combine to form an optionally substituted C₁₋₃ bridge, optionally substituted C₃₋₆cycloalkyl or optionally substituted 5- to 7-membered N-heterocycloalkyl, optionally wherein the C₃₋₅cycloalkyl or the 5-7-membered N-heterocycloalkyl are joined to ring A at a spiro centre.

In some examples, both of R¹ and R² are present. For example, in some cases, R² is present and at least one R¹ is selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R¹ groups combine to form a optionally substituted C₁₋₃ bridge, optionally substituted C₃₋₆cycloalkyl or optionally substituted 5- to 7-membered N-heterocycloalkyl.

In the compounds described herein, R¹ and/or R² may be covalently attached to the heterocyclic ring (e.g. ring A) at any suitable position e.g. provided it has the correct valency and/or is chemically suitable. For example, R¹ and/or R² may replace a hydrogen atom at any position on the heterocyclic core, e.g. that shown in formula (I).

Where both R¹ and R² are present, they may be covalently attached to the heterocyclic ring (e.g. ring A) at the same or different positions. For example, in some cases R¹ and R² may be covalently attached to the heterocyclic core by way of different carbon atoms. In other cases, R¹ and R² may be covalently attached to the heterocyclic core by way of the same carbon atom.

As shown in the formulae described herein in relation to the Z moiety, a double bond is present in Z. The stereochemistry of this double bond may be either E or Z and this is indicated by the wavy line bond in formula (I) (and is similarly shown on the other formulae and structures disclosed herein). The designation of this moiety as either E or Z may depend on the identity of the R³ group. In some examples, Z may comprise a mixture of E and Z stereoisomers. Thus, the present disclosure includes within its scope the use of each individual E and Z stereoisomers of any of the disclosed Z moieties according to formula (I) and any of the other formulae described herein (e.g. in a substantially stereopure form), as well as the use of mixtures of these E and Z isomers. In some cases, the stereochemistry of the double bond and the moieties bound to it is Z, i.e. the Z stereoisomer. In other examples, the stereochemistry of the double bond and the moieties bound to it is E, i.e. the E stereoisomer.

For the avoidance of doubt, where the vinylic double bond of Z, for example that of formula (I), is shown in a structure herein to be a specific stereoisomer (E or Z) in any of the specific examples of this disclosure, it need not be in that specific stereoisomer. In other words, both E and Z steroisomers and mixtures of the two are included within the scope of the structure irrespective of the specific stereoisomer shown.

By way of further example, Z may be represented as either formula (Ia) or (Ib):

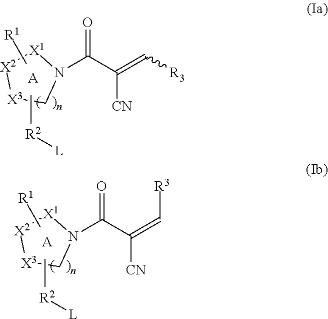

wherein R¹, R², R³, X¹, X², X³ and n are as defined above and herein.

It will be appreciated that the bifunctional molecules of the present disclosure may exist in different stereoisomeric forms. The present disclosure includes within its scope the use of all stereoisomeric forms, or the use of a mixture of stereoisomers of the bifunctional molecules, By way of example, where the bifunctional molecule comprises one or more chiral centres, the present disclosure encompasses each individual enantiomer of the bifunctional molecule as well as mixtures of enantiomers including racemic mixtures of such enantiomers. By way of further example, where the bifunctional molecule comprises two or more chiral centres, the present disclosure encompasses each individual diastereomer of the bifunctional molecule, as well as mixtures of the various diastereomers.

Unless otherwise indicated, the various structures shown herein encompass all isomeric (e.g. enantiomeric, diastereomeric, and geometric (or conformational)) forms of the structure). For example, the present disclosure embraces the R and S configurations for each asymmetric centre, and Z and E double bond isomers. Therefore, single stereochemical isomers as well as enantiomeric, diastereomeric, and geometric (or conformational) mixtures of the present compounds are to be understood to be within the scope of the present disclosure. Additionally, unless otherwise stated, where present, all tautomeric forms of the bifunctional molecules described herein are to be understood to be within the scope of the present disclosure.

Additionally, unless otherwise stated, structures depicted herein are also meant to include compounds that differ only in the presence of one or more isotopically enriched atoms. For example, bifunctional molecules as described herein in which one or more hydrogen atoms have been replaced by deuterium or tritium, or in which one or more carbon atoms have been replaced by a ¹³C— or ¹⁴C-enriched carbon are to be understood to within the scope of the present disclosure. Such molecules may be useful, for example, as analytical tools, as probes in biological assays, or as therapeutic agents in accordance with the present disclosure. By way of further example, a bifunctional molecule as described herein, may be substituted with one or more deuterium atoms.

As used herein, references to “a bifunctional molecule” may further embrace a pharmaceutically acceptable salt thereof.

By way of further example, Z may be represented as formula (Ic′):

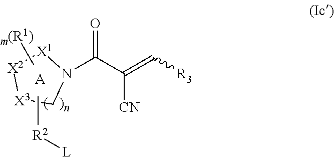

wherein:
R¹ is absent (i.e. m is 0) or is selected from the group consisting of: aryl having 6 to 10 carbon ring atoms that is optionally substituted with one to three substituents; heteroaryl having 5 to 10 ring atoms containing 1 to 3 heteroatoms each independently selected from N, O and S, the heteroaryl being optionally substituted with one to three substituents; C₃ to C₈ cycloalkyl being optionally substituted with one to three substituents; heterocycloalkyl having 3 to 10 ring atoms and containing 1 to 3 ring heteroatoms each independently selected from N, O and S, the heterocycloalkyl being optionally substituted with one to three substituents; C₁ to C₈ alkyl optionally substituted with one to three substituents; and/or wherein two R¹ groups combine to form a C₁₋₃ bridge optionally substituted with one to three substituents, C₃₋₅cycloalkyl optionally substituted with one to three substituents or 5- to 7-membered N-heterocycloalkyl optionally substituted with one to three substituents (e.g wherein the C₃₋₅cycloalkyl or the 5-7-membered N-heterocycloalkyl are joined to ring A at a spiro centre);
R² is absent or is selected from the group consisting of: aryl having 6 to 10 carbon ring atoms, the aryl being optionally substituted with one to three substituents; heteroaryl having 5 to 10 ring atoms and containing 1 to 3 heteroatoms each independently selected from N, O and S, the heteroaryl being optionally substituted with one to three substituents; heterocycloalkyl having 3 to 10 ring atoms and containing 1 to 3 heteroatoms each independently selected from N, O and S, the heterocycloalkyl being optionally substituted with one to three substituents; —NR^{y}; —CH(aryl)-, wherein the aryl has 6 to 10 carbon ring atoms and is optionally substituted with one to three substituents)-; and —CH(heteroaryl)-, wherein the heteroaryl has 5 to 10 ring atoms and contains 1 to 3 heteroatoms each independently selected from N, O and S, the heteroaryl being optionally substituted with one to three substituents;
wherein R^{y} is H or C₁ to C₆ alkyl;
R³ is selected from the group consisting of: C₁ to C₆ alkyl optionally substituted with one to three substituents; C₃ to C₈ cycloalkyl optionally substituted with one to three substituents; heterocycloalkyl having 3 to 10 ring atoms and containing 1 to 3 heteroatoms each independently selected from N, O and S, the heterocycloalkyl being optionally substituted with one to three substituents; aryl having 6 to 10 carbon ring atoms, the aryl being optionally substituted with one to three substituents; heteroaryl having 5 to 10 ring atoms and containing 1 to 3 heteroatoms each independently selected from N, O and S, the heteroaryl being optionally substituted with one to three substituents;
X¹ is CH₂;
X² and X³ are each independently CH₂, or a heteroatom selected from O and NR^{x}, wherein R^{x} is H or C₁ to C₆ alkyl, or wherein one R¹ group and one R^{x} group combine to form a C₁₋₃ bridge optionally substituted with one to three substituents; with the proviso that none, or only 1 or 2 X² and X³ is a heteroatom; and
m is 0, 1, 2 or 3;
n is 0, 1, 2, or 3; and
L shows the point of attachment of the linker.

By way of further example, Z may be represented as formula (Ic):

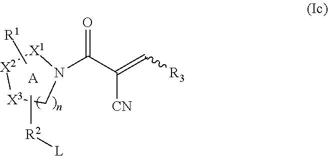

wherein:
R¹ is absent or is selected from the group consisting of: aryl having 6 to 10 carbon ring atoms that is optionally substituted with one to three substituents; heteroaryl having 5 to 10 ring atoms containing 1 to 3 heteroatoms each independently selected from N, O and S, the heteroaryl being optionally substituted with one to three substituents; C₃ to C₈ cycloalkyl; C₁ to Ce alkyl optionally substituted with one to three substituents;
R² is absent or is selected from the group consisting of: aryl having 6 to 10 carbon ring atoms, the aryl being optionally substituted with one to three substituents; heteroaryl having 5 to 10 ring atoms and containing 1 to 3 heteroatoms each independently selected from N, O and S, the heteroaryl being optionally substituted with one to three substituents; heterocycloalkyl having 3 to 10 ring atoms and containing 1 to 3 heteroatoms each independently selected from N, O and S, the heterocycloalkyl being optionally substituted with one to three substituents; —NR^{y}; —CH(aryl)-, wherein the aryl has 6 to 10 carbon ring atoms and is optionally substituted with one to three substituents)-; and —CH(heteroaryl)-, wherein the heteroaryl has 5 to 10 ring atoms and contains 1 to 3 heteroatoms each independently selected from N, O and S, the heteroaryl being optionally substituted with one to three substituents;
wherein R^{y} is H or C₁ to C₆ alkyl;
R³ is selected from the group consisting of: C₁ to C₆ alkyl optionally substituted with one to three substituents; C₃ to C₈ cycloalkyl optionally substituted with one to three substituents; heterocycloalkyl having 3 to 10 ring atoms and containing 1 to 3 heteroatoms each independently selected from N, O and S, the heterocycloalkyl being optionally substituted with one to three substituents; aryl having 6 to 10 carbon ring atoms, the aryl being optionally substituted with one to three substituents; heteroaryl having 5 to 10 ring atoms and containing 1 to 3 heteroatoms each independently selected from N, O and S, the heteroaryl being optionally substituted with one to three substituents;
X¹ is CH₂;
X² and X³ are each independently CH₂, or a heteroatom selected from O and NR^{x}, wherein R^{x} is H or C₁ to C₆ alkyl; with the proviso that none, or only 1 or 2 X² and X³ is a heteroatom; and
n is 0, 1, 2, or 3; and
L shows the point of attachment of the linker.

By way of further example, Z may be represented as formula (Id′):

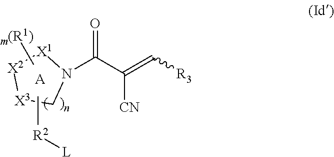

wherein:
R¹ is absent (i.e. when m is 0) or is selected from the group consisting of: phenyl that is optionally substituted with one to three substituents selected from the group consisting of halo, C₁ to C₆ alkyl, C₁ to C₆ haloalkyl and C₁ to C₆ alkoxy; heteroaryl having 5 to 6 ring atoms containing 1 to 3 heteroatoms each independently selected from N, O and S, the heteroaryl being optionally substituted with one to three substituents selected from the group consisting of halo, C₁ to C₆ alkyl, C₁ to C₆ haloalkyl and C₁ to C₆ alkoxy; heterocycloalkyl having 5 to 7 ring atoms and containing 1 to 3 ring heteroatoms each independently selected from N, O and S; C₃ to C₈ cycloalkyl; C₁ to C₆ alkyl and C₁ to C₆ haloalkyl;

and/or wherein two R¹ groups combine to form a C₁₋₃ bridge, C₃₋₅cycloalkyl or 5- to 7-membered N-heterocycloalkyl (e.g, wherein the C₃₋₅cycloalkyl or the 5-7-membered N-heterocycloalkyl are joined to ring A at a spiro centre);

R² is absent or is selected from the group consisting of: phenyl that is optionally substituted with one to three substituents selected from the group consisting of halo, C₁ to C₆ alkyl, C₁ to C₆ haloalkyl and C₁ to C₆ alkoxy; heteroaryl having 5 to 6 ring atoms containing 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to C₆ alkoxy; heterocycloalkyl having 5 to 7 ring atoms and containing 1 to 3 heteroatoms each independently selected from N, O and S, the heterocycloalkyl being optionally substituted with one to three substituents each independently selected from the group consisting of halo, C₁ to C₆ alkyl, C₁ to C₆ haloalkyl and C₁ to C₆ alkoxy; —NR^{y}; —CH(phenyl)-, wherein the phenyl is optionally substituted with one to three substituents each independently selected from the group consisting of halo, C₁ to C₆ alkyl, C₁ to C₆ haloalkyl and C₁ to C₆ alkoxy; and —CH(heteroaryl), wherein the heteroaryl has 5 to 6 ring atoms and contains 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to C₆ alkoxy;
wherein R^{y} is H or C₁ to C₆ alkyl;
R³ is selected from the group consisting of C₁ to C₆ alkyl optionally wherein the C₁ to C₆ alkyl is substituted with a heterocycloalkyl group; C₃ to C₆ cycloalkyl optionally wherein the C₃ to C₆ cycloalkyl is substituted with one to three substituents each independently selected from the group consisting of halo, C₁ to C₆ alkyl, C₁ to C₆ haloalkyl and C₁ to C₆ alkoxy; phenyl that is optionally substituted with one to three substituents each independently selected from the group consisting of halo, C₁ to C₆ alkyl, C₁ to C₆ haloalkyl and C₁ to C₆ alkoxy; and heteroaryl having 5 to 6 ring atoms containing 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to C₆ alkoxy;
X¹ is CH₂;
X² and X³ are each independently CH₂, or a heteroatom selected from O and NR^{x}, wherein R^{x} is H or C₁ to C₆ alkyl, or wherein one R¹ group and one R^{x} group combine to form a C₁₋₃ bridge; with the proviso that none or only 1 of X² and X³ is a heteroatom; and
m is 0, 1, 2 or 3;
n is 0, 1, 2, or 3; and
L shows the point of attachment of the linker.

By way of further example, Z may be represented as formula (Id):

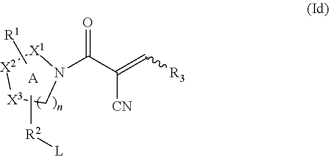

wherein:
R¹ is absent or is selected from the group consisting of: phenyl that is optionally substituted with one to three substituents selected from the group consisting of halo, C₁ to C₆ alkyl, C₁ to C₆ haloalkyl and C₁ to C₆ alkoxy; heteroaryl having 5 to 6 ring atoms containing 1 to 3 heteroatoms each independently selected from N, O and S, the heteroaryl being optionally substituted with one to three substituents selected from the group consisting of halo, C₁ to C₆ alkyl, C₁ to Ce haloalkyl and C₁ to C₆ alkoxy; C₃ to C₈ cycloalkyl; C₁ to C₆ alkyl and C₁ to C₆ haloalkyl;
R² is absent or is selected from the group consisting of: phenyl that is optionally substituted with one to three substituents selected from the group consisting of halo, C₁ to C₆ alkyl, C₁ to C₆ haloalkyl and C₁ to C₆ alkoxy; heteroaryl having 5 to 6 ring atoms containing 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to C₆ alkoxy; heterocycloalkyl having 5 to 7 ring atoms and containing 1 to 3 heteroatoms each independently selected from N, O and S, the heterocycloalkyl being optionally substituted with one to three substituents each independently selected from the group consisting of halo, C₁ to C₆ alkyl, C₁ to C₆ haloalkyl and C₁ to C₆ alkoxy; —NR^{y}; —CH(phenyl)-, wherein the phenyl is optionally substituted with one to three substituents each independently selected from the group consisting of halo, C₁ to C₆ alkyl, C₁ to C₆ haloalkyl and C₁ to C₆ alkoxy; and —CH(heteroaryl), wherein the heteroaryl has 5 to 6 ring atoms and contains 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to C₆ alkoxy;
wherein R^{y} is H or C₁ to C₆ alkyl;
R³ is selected from the group consisting of C₁ to C₆ alkyl optionally wherein the C₁ to C₆ alkyl is substituted with a heterocycloalkyl group; C₃ to C₈ cycloalkyl optionally substituted with one to three substituents; heterocycloalkyl having 3 to 10 ring atoms and containing 1 to 3 heteroatoms each independently selected from N, O and S, the heterocycloalkyl being optionally substituted with one to three substituents; phenyl that is optionally substituted with one to three substituents each independently selected from the group consisting of halo, C₁ to C₆ alkyl, C₁ to Ce haloalkyl and C₁ to C₆ alkoxy; and heteroaryl having 5 to 6 ring atoms containing 1 to 3 heteroatoms each independently selected from N, O and S, the heteroaryl being optionally substituted with one to three substituents each independently selected from the group consisting of halo, C₁ to C₆ alkyl, C₁ to C₆ haloalkyl and C₁ to Ce alkoxy;
X¹ is CH₂;
X² and X³ are each independently CH₂, or a heteroatom selected from O and NR^{x}, wherein R^{x} is H or C₁ to C₆ alkyl; with the proviso that none or only 1 of X² and X³ is a heteroatom; and
n is 0, 1, 2, or 3; and
L shows the point of attachment of the linker.

By way of further example, Z may be represented as formula (Ie′):

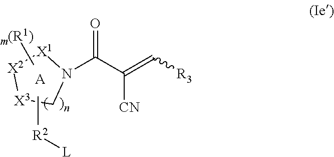

wherein:
R¹ is absent (i.e. when m is 0) or is selected from the group consisting of: phenyl; heteroaryl having 5 to 6 ring atoms containing 1 or 2 heteroatoms each independently selected from N, O and S; C₃ to C₇ cycloalkyl; heterocycloalkyl having 5 to 7 ring atoms and containing 1 or 2 heteroatoms each independently selected from N, O and S; C₁ to C₆ alkyl and C₁ to C₆ haloalkyl; wherein the phenyl or heteroaryl is optionally substituted with one substituent selected from the group consisting of halo, C₁ to C₃ alkyl, C₁ to C₃ haloalkyl and C₁ to C₃ alkoxy; and/or wherein two R¹ groups combine to form a C₁₋₃ bridge, C₃₋₅cycloalkyl or 5- to 7-membered N-heterocycloalkyl (e.g, wherein the C₃₋₅cycloalkyl or the 5- to 7-membered N-heterocycloalkyl are joined to ring A at a spiro centre);
R² is absent or is selected from the group consisting of: phenyl; heteroaryl having 5 to 6 ring atoms and containing 1 or 2 heteroatoms each independently selected from N, O and S; heterocycloalkyl having 5 to 7 ring atoms and containing 1 or 2 heteroatoms each independently selected from N, O and S; —NR^{y}; —CH(phenyl)-; and —CH(heteroaryl) wherein the heteroaryl has 5 to 6 ring atoms and contains 1 or 2 heteroatoms each independently selected from N, O and S; and further wherein the phenyl, heteroaryl, heterocycloalkyl, —CH(phenyl)- and —CH(heteroaryl) are each optionally substituted with one substituent selected from the group consisting of halo, C₁ to C₃ alkyl, C₁ to C₃ haloalkyl and C₁ to C₃ alkoxy;
wherein R^{y} is H or C₁ to C₆ alkyl;
R³ is selected from the group consisting of C₁ to C₆ alkyl optionally wherein the C₁ to C₆ alkyl is substituted with a heterocycloalkyl group the heterocycloalkyl having 5 to 7 ring atoms and containing 1 or 2 heteroatoms each independently selected from N, O and S; C₃ to C₆ cycloalkyl; phenyl; and heteroaryl having 5 to 6 ring atoms containing 1 to 3 heteroatoms each independently selected from N, O and S; wherein the C₃ to C₆ cycloalkyl, phenyl and heteroaryl are optionally substituted with one or two substituents selected from the group consisting of halo, C₁ to C₃ alkyl, C₁ to C₃ haloalkyl and C₁ to C₃ alkoxy;
X¹ is CH₂;
X² and X³ are each independently CH₂ or O; with the proviso that none or only 1 of X² and X³ is O;
m is 0, 1, 2 or 3;
n is 1, 2, or 3; and
L shows the point of attachment of the linker.

By way of further example, Z may be represented as formula (Ie):

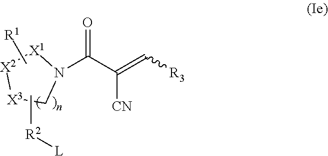

wherein:
R¹ is absent or is selected from the group consisting of: phenyl; heteroaryl having 5 to 6 ring atoms containing 1 or 2 heteroatoms each independently selected from N, O and S; C₃ to C₇ cycloalkyl; C₁ to C₆ alkyl and C₁ to C₆ haloalkyl; wherein the phenyl or heteroaryl is optionally substituted with one substituent selected from the group consisting of halo, C₁ to C₃ alkyl, C₁ to C₃ haloalkyl and C₁ to C₃ alkoxy;
R² is absent or is selected from the group consisting of: phenyl; heteroaryl having 5 to 6 ring atoms and containing 1 or 2 heteroatoms each independently selected from N, O and S; heterocycloalkyl having 5 to 7 ring atoms and containing 1 or 2 heteroatoms each independently selected from N, O and S; —NR^{y}; —CH(phenyl)-; and —CH(heteroaryl) wherein the heteroaryl has 5 to 6 ring atoms and contains 1 or 2 heteroatoms each independently selected from N, O and S; and further wherein the phenyl, heteroaryl, heterocycloalkyl, —CH(phenyl)- and —CH(heteroaryl) are each optionally substituted with one substituent selected from the group consisting of halo, C₁ to C₃ alkyl, C₁ to C₃ haloalkyl and C₁ to C₃ alkoxy;
wherein R^{y} is H or C₁ to C₆ alkyl;
R³ is selected from the group consisting of C₁ to C₆ alkyl optionally wherein the C₁ to C₆ alkyl is substituted with a heterocycloalkyl group the heterocycloalkyl having 5 to 7 ring atoms and containing 1 or 2 heteroatoms each independently selected from N, O and S; C₃ to C₆ cycloalkyl; phenyl; and heteroaryl having 5 to 6 ring atoms containing 1 to 3 heteroatoms each independently selected from N, O and S; wherein the C₃ to C₆ cycloalkyl, phenyl and heteroaryl are optionally substituted with one or two substituents selected from the group consisting of halo, C₁ to C₃ alkyl, C₁ to C₃ haloalkyl and C₁ to C₃ alkoxy;
X¹ is CH₂;
X² and X³ are each independently CH₂ or O; with the proviso that none or only 1 of X² and X³ is O;
and
n is 1, 2, or 3; and
L shows the point of attachment of the linker.

In further embodiments, Z comprises a structure according to formula (ZII):

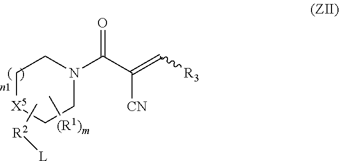

wherein R² is absent or is as described in any one of the embodiments disclosed herein;
R³ is as described in any one of the embodiments disclosed herein;
X⁵ is CR^{b}₂, NR^{b}, O or a 5- to 7-membered heterocycloalkyl (e.g. a 5- to 7-membered heterocycloalkyl);
each R¹ is independently selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R¹ groups combine to form an optionally substituted C₁₋₃ bridge or optionally substituted C₃₋₅cycloalkyl (optionally wherein the C₃₋₅cycloalkyl is joined to the heterocyclic ring shown in formula (ZII) at a spiro centre);
R^{b} is H or optionally substituted C₁₋₃alkyl;
n1 is 0, 1, 2 or 3;
m is 0, 1 or 2; and
L shows the point of attachment of the linker.

In yet further embodiments, Z comprises a structure according to any one of formulae (ZIIa) to (ZIIe):

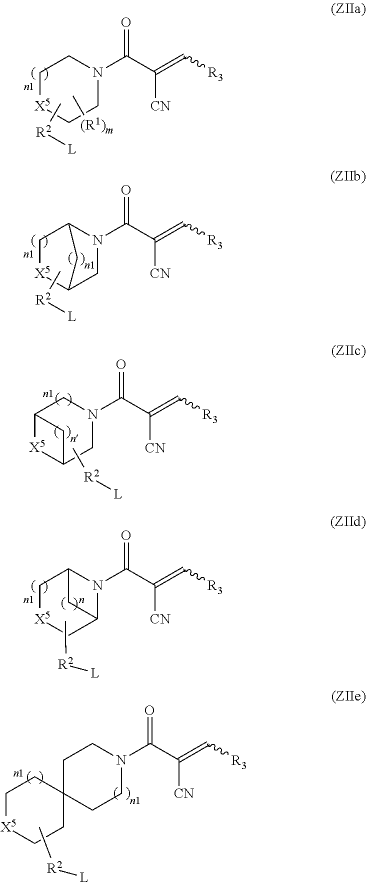

wherein:
R² is as described in any one of the embodiments disclosed herein;
R³ is as described in any one of the embodiments disclosed herein;
each R¹ is independently selected from aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R¹ groups combine to form an optionally substituted C₃₋₅cycloalkyl (optionally wherein the C₃₋₅cycloalkyl is joined to the heterocyclic ring shown in formula (ZIIa) at a spiro centre);
X⁵ is C(R^{b})₂, NR^{b} or O;
R^{b} is H or optionally substituted C₁₋₃alkyl;
n1 is 0, 1, 2 or 3;
n′ is 1 or 2;
m is 0, 1 or 2; and
L shows the point of attachment of the linker.

For example, Z may comprise a structure according to formula (ZIIIa) to (ZIIIh):

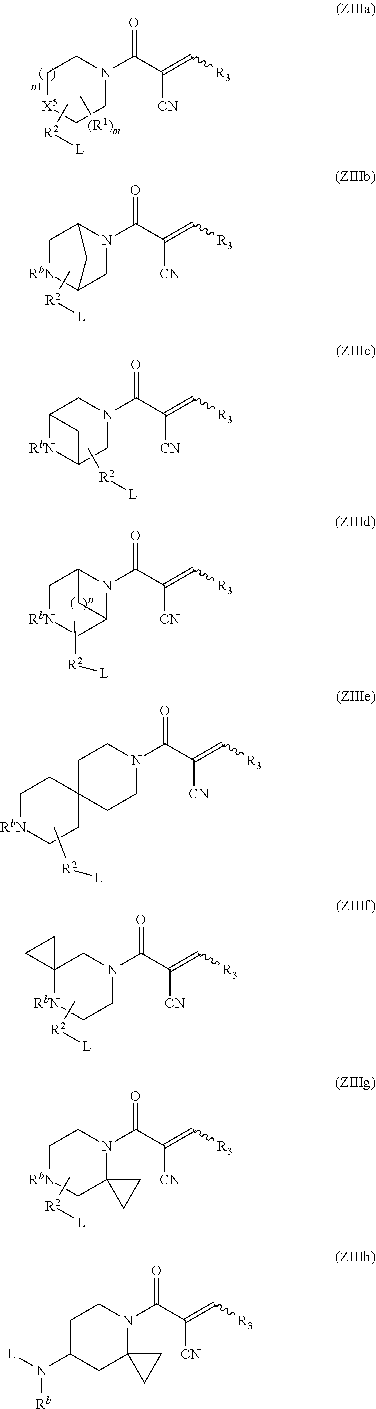

wherein:
R² is as described in any one of the embodiments disclosed herein;
R³ is as described in any one of the embodiments disclosed herein;
each R¹ is independently selected from aryl, substituted aryl, heteroaryl, substituted heteroaryl, cycloalkyl, substituted cycloalkyl, C₁ to C₆ alkyl and substituted C₁ to C₆ alkyl;
X⁵ is CH₂, NR^{b} or O;
R^{b} is H or optionally substituted C₁₋₃alkyl;
n1 is 0, 1 or 2;
n′ is 1 or 2;
m is 0, 1 or 2; and
L shows the point of attachment of the linker.

In even further embodiments, Z comprises a structure according to formula (ZIVa) to (ZIVj):

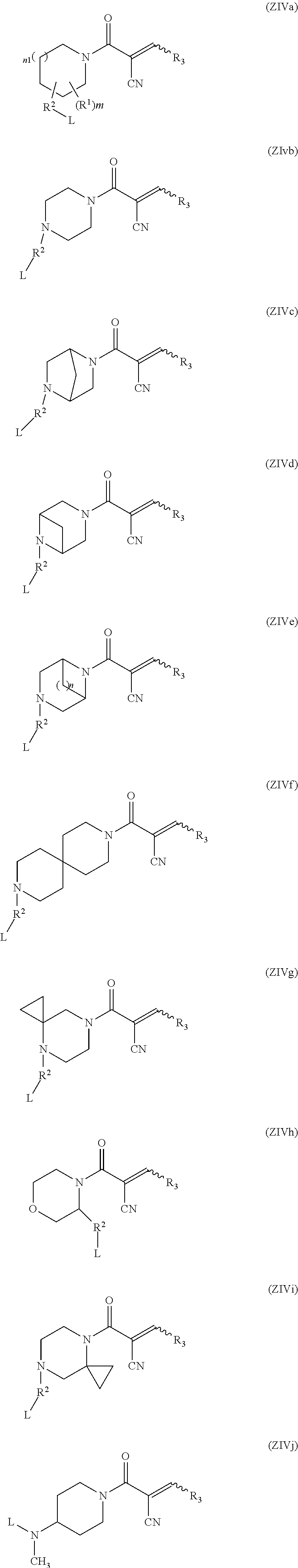

wherein:
R² is absent or is as described in any one of the embodiments disclosed herein;
R³ is as described in any one of the embodiments disclosed herein;
each R¹ is independently selected from aryl, substituted aryl, heteroaryl, substituted heteroaryl, cycloalkyl, substituted cycloalkyl, C₁ to C₆ alkyl and substituted C₁ to C₆ alkyl;
n1 is 0, 1 or 2;
n′ is 1 or 2;
m is 0, 1 or 2; and
L shows the point of attachment of the linker.

In further examples, Z comprises a structure according to formula (If):

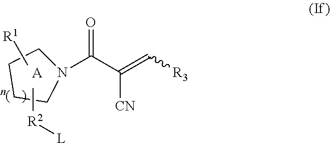

wherein R¹ is absent or is selected from aryl, substituted aryl, heteroaryl, substituted heteroaryl, cycloalkyl, C₁ to C₆ alkyl and substituted C₁ to C₆ alkyl;
R² is absent or is selected from aryl, substituted aryl, heteroaryl, substituted heteroaryl, heterocycloalkyl, substituted heterocycloalkyl, —CH(aryl)- and —CH(substituted aryl)-;
R³ is selected from C₁ to C₆ alkyl, aryl, heteroaryl, substituted C₁ to C₆ alkyl, substituted aryl, and substituted heteroaryl; and
wherein at least one of R¹ and R² is present;
n is 0, 1, 2, or 3; and
L shows the point of attachment of the linker.

In some examples, R¹, R² and R³ of formula (If) may be selected from those groups defined above, e.g. for any one or more of formulae (Ic′), (Ic), (Id′), (Id), (Ie′) or (Ie).

In some examples of the formulae described above and herein, n may be 1, 2 or 3 and/or n1 may be 0, 1 or 2.

In those cases where R¹ is absent, Z may be represented by formula (II):

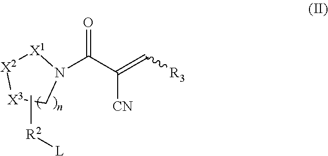

wherein R² is selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl);
R³ is selected from C₁ to C₆ alkyl, aryl, heteroaryl, substituted aryl, and substituted heteroaryl, optionally wherein the C₁ to C₆ alkyl is substituted with a heterocycloalkyl group;
X¹ is CH₂;
X² and X³ are each independently CH₂ or O; with the proviso that none or only 1 of X² and X³ is O; and
n is 0, 1, 2 or 3; and
L shows the point of attachment of the linker;
and wherein Z is not:

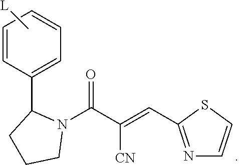

In those cases where R¹ is absent, Z may be represented by formula (IIa):

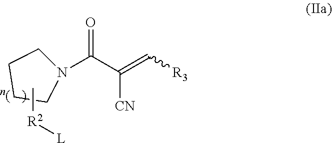

wherein R² is selected from aryl, substituted aryl, heteroaryl, substituted heteroaryl, heterocycloalkyl, substituted heterocycloalkyl, —CH(aryl)-, —CH(substituted aryl)-, —CH(heteroaryl)- and —CH(substituted heteroaryl);
R³ is selected from C₁ to C₆ alkyl, aryl, heteroaryl, substituted aryl, and substituted heteroaryl, optionally wherein the C₁ to C₆ alkyl is substituted with a a heterocycloalkyl group; and
n is 0, 1, 2 or 3; and
L shows the point of attachment of the linker;
and wherein Z is not:

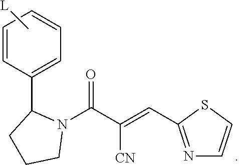

By way of particular example, in formulae (II) or (IIa), n may be 1 or 2.

By way of further example, Z may be represented by formula (IIb):

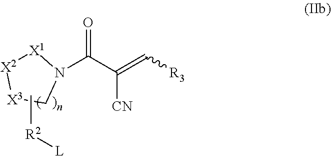

wherein R² is selected from aryl substituted aryl, heteroaryl, substituted heteroaryl, heterocycloalkyl, and substituted heterocycloalkyl;
R³ is selected from C₁ to C₆ alkyl, aryl, heteroaryl, substituted aryl, and substituted heteroaryl, optionally wherein the C₁ to C₆ alkyl is substituted with a heterocycloalkyl group;
X¹ is CH₂;
X² and X³ are each independently CH₂ or O; with the proviso that none or only 1 of X² and X³ is O;
n is 1 or 2; and
L shows the point of attachment of the linker;
and wherein Z is not:

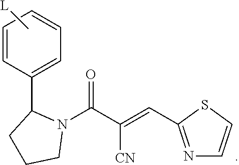

By way of further example, Z may be represented by formula (IIc):

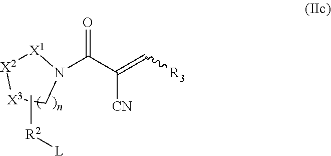

wherein R² is selected from heterocycloalkyl and substituted heterocycloalkyl;
R³ is selected from C₁ to C₆ alkyl, aryl, heteroaryl, substituted aryl, and substituted heteroaryl, optionally wherein the C₁ to C₆ alkyl is substituted with a heterocycloalkyl group;
X¹ is CH₂;
X² and X³ are each independently CH₂ or O; with the proviso that none or only 1 of X² and X³ is O;
n is 1 or 2; and
L shows the point of attachment of the linker.

In some cases, Z may be represented by formula (IId):

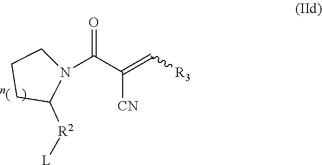

wherein R² is selected from heterocycloalkyl and substituted heterocycloalkyl;
R³ is selected from C₁ to C₆ alkyl, aryl, heteroaryl, substituted aryl, and substituted heteroaryl, optionally wherein the C₁ to C₆ alkyl is substituted with a heterocycloalkyl group;
n is 1 or 2; and
L shows the point of attachment of the linker.

In other examples, Z may comprise a structure according to formula (IIe):

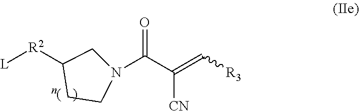

wherein R² is selected from aryl, substituted aryl, heteroaryl, substituted heteroaryl, heterocycloalkyl and substituted heterocycloalkyl;
R³ is selected from C₁ to C₆ alkyl, aryl, heteroaryl, substituted aryl, and substituted heteroaryl, optionally wherein the C₁ to C₆ alkyl is substituted with a heterocycloalkyl group;
n is 1 or 2; and
L shows the point of attachment of the linker.

In other examples, Z may comprise a structure according to formula (IIf):

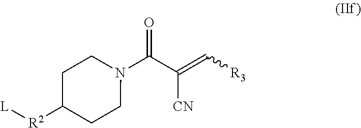

wherein R² is selected from aryl, substituted aryl, heteroaryl, substituted heteroaryl, heterocycloalkyl and substituted heterocycloalkyl;
R³ is selected from C₁ to C₆ alkyl, aryl, heteroaryl, substituted aryl, and substituted heteroaryl, optionally wherein the C₁ to C₆ alkyl is substituted with a heterocycloalkyl group; and
L shows the point of attachment of the linker.

In those cases where R² is absent, Z may comprise a structure according to formula (III):

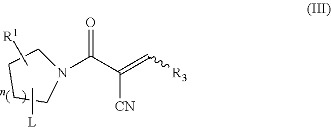

wherein R¹ is selected from aryl, substituted aryl, heteroaryl, substituted heteroaryl, cycloalkyl and C₁ to C₆ alkyl;
R³ is selected from C₁ to C₆ alkyl, aryl, heteroaryl, substituted aryl, and substituted heteroaryl, optionally wherein the C₁ to C₆ alkyl is substituted with a heterocycloalkyl group; and
n is 0, 1, 2 or 3; and
L shows the point of attachment of the linker.

In some examples, n may be 1 or 2.

In some examples where n is 2, Z may be represented by formula (IIIa):

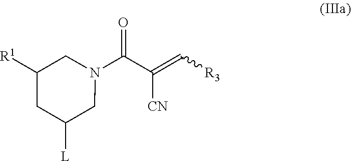

wherein R¹ is selected from aryl, substituted aryl, heteroaryl, substituted heteroaryl, cycloalkyl and C₁ to C₆ alkyl;
R³ is selected from C₁ to C₆ alkyl, aryl, heteroaryl, substituted aryl, and substituted heteroaryl, optionally wherein the C₁ to C₆ alkyl is substituted with a heterocycloalkyl group; and
L shows the point of attachment of the linker.

In some examples where n is 1, Z may be represented by formula (IIIb):

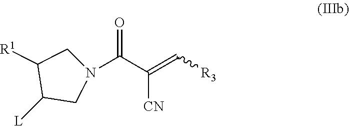

wherein R¹ is selected from aryl, substituted aryl, heteroaryl, substituted heteroaryl, cycloalkyl and C₁-C₆ alkyl;
R³ is selected from C₁ to C₆ alkyl, aryl, heteroaryl, substituted aryl, and substituted heteroaryl, optionally wherein the C₁ to C₆ alkyl is substituted with a heterocycloalkyl group; and
L shows the point of attachment of the linker.

As illustrated above, bifunctional molecules of formula (IIIb) comprise at least two stereocentres and so exist in several diastereomeric (and enantiomeric) forms. In some examples, the groups R¹ and L may exist in a trans relationship (e.g. these groups are held and/or oriented on opposite sides of the heterocyclic core). In other examples, the groups R¹ and L may exist in a cis relationship (e.g. these groups are held and/or oriented on the same side of the heterocyclic core). By way of further example, bifunctional molecules of formula (IIIb) may encompass at least the following diastereomeric forms:

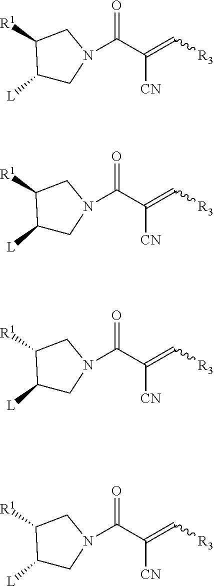

In those examples where R¹ is absent and R² is selected from CH(aryl)-, —CH(substituted aryl)-, —CH(heteroaryl)- and —CH(substituted heteroaryl)-, Z may be represented by formula (IV):

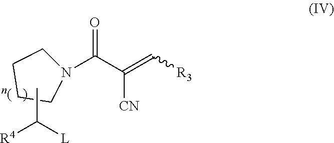

wherein R³ is selected from C₁ to C₆ alkyl, aryl, heteroaryl, substituted aryl, and substituted heteroaryl, optionally wherein the C₁ to C₆ alkyl is substituted with a heterocycloalkyl group;
R⁴ is selected from aryl, substituted aryl, heteroaryl and substituted heteroaryl; and
n is 0, 1, 2 or 3; and
L shows the point of attachment of the linker.

In some examples, Z may comprise a structure according to formula (IVa):

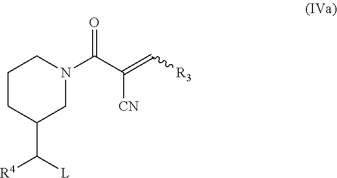

wherein R³ is selected from C₁ to C₆ alkyl, aryl, heteroaryl, substituted aryl, and substituted heteroaryl, optionally wherein the C₁ to C₆ alkyl is substituted with a heterocycloalkyl group;
R⁴ is selected from aryl, substituted aryl, heteroaryl and substituted heteroaryl; and
L shows the point of attachment of the linker.

In either of formula (IV) or (IVa), R⁴ may be selected from aryl or substituted aryl.

Representative examples of groups R¹, R², R³ and R⁴ are now provided below which are applicable to any one or more of the formulae described herein (unless otherwise indicated).

With respect to the various structures for Z defined by the formulae herein (and unless otherwise stated), R¹ may be selected from aryl, substituted aryl, heteroaryl, substituted heteroaryl, cycloalkyl, substituted cycloalkyl, C₁ to C₆ alkyl, and substituted C₁ to C₆ alkyl.

In some examples, R¹ is an optionally substituted aryl or an optionally substituted heteroaryl. Where R¹ is a substituted aryl or substituted heteroaryl, the aryl or heteroaryl may comprise one or more substituents selected from the group consisting of C₁ to C₆ alkyl (e.g. methyl), C₁ to C₆ alkoxy (e.g. methoxy), C₁ to C₆ haloalkyl and halo.

By way of further example, R¹ may be phenyl that is optionally substituted with one to three substituents selected from the group consisting of halo, C₁ to C₆ alkyl, C₁ to C₆ haloalkyl and C₁ to C₆ alkoxy. By way of a yet further example, R¹ may be heteroaryl having 5 to 6 ring atoms containing 1 to 3 heteroatoms each independently selected from N, O and S, the heteroaryl being optionally substituted with one to three substituents selected from the group consisting of halo, C₁ to C₆ alkyl, C₁ to C₆ haloalkyl and C₁ to C₈ alkoxy; C₃ to C₈ cycloalkyl.

Representative examples of suitable R¹ groups include but are not limited to phenyl, substituted phenyl, pyrazolyl, and substituted pyrazolyl.

In some examples, R¹ is a cycloalkyl, such as a C₃ to C₇ cycloalkyl, or a C₃ to C₆ cycloalkyl.

In some examples, R¹ is a C₁ to C₆ alkyl, such as a C₁ to C₃ alkyl that is optionally substituted with one to three substituents as defined herein.

Further non-limiting examples of suitable R¹ groups are illustrated below:

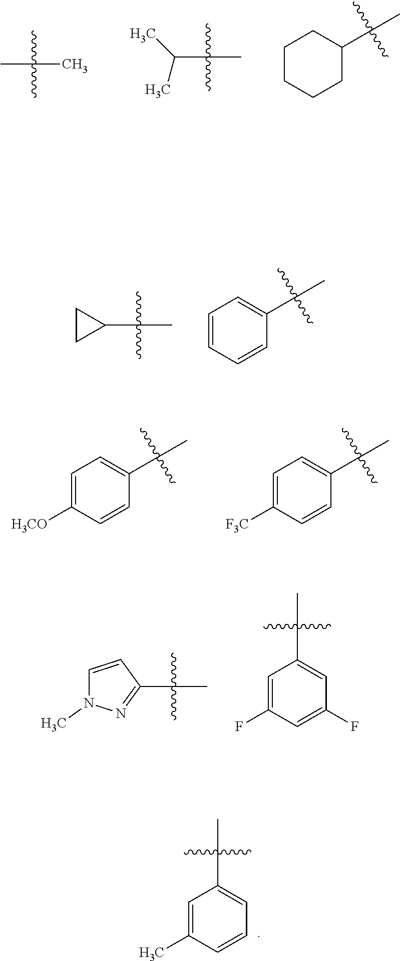

In the structures shown above, the line intersected by a wavy line represents the covalent bond between the exemplary R¹ groups shown above and a carbon atom on the heterocycloalkyl core attached to the R¹ group in the parent structure of Z (as illustrated by the various formulae described herein). Although a particular substitution pattern is shown in the exemplary aryl and heteroaryl structures above, it will be appreciated that other substitution patterns are also encompassed within the scope of the present disclosure.

In further examples, such as in respect of formulae (ZII), two R¹ groups may combine to form a C₁₋₃ bridge or C₃₋₅cycloalkyl. For example, two R¹ groups may combine to form a C₃₋₅cycloalkyl. In such examples, the C₃₋₅cycloalkyl may be joined to the heterocyclic ring of the parent structure at a spiro centre.

With respect to the various structures for Z defined by the formulae herein (and unless otherwise stated), R² may be selected from aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl) and —CH(substituted heteroaryl); wherein R^{y} is optionally substituted C₁₋₆alkyl (such as methyl) or H

In some examples, R² is present in Z (and/or the bifunctional molecules described herein) as a divalent group. In other words, as shown in formulae (I) to (IVa) (and unless otherwise stated), the various groups defined for R² are covalently attached to an atom of the heterocyclic core of Z and also may be covalently attached to an atom of a linker. Thus, these groups may be considered as divalent radical species.

Where R² is selected from optionally substituted aryl and optionally substituted heteroaryl, R² may be selected from aryl having 6 to 10 carbon ring atoms, the aryl being optionally substituted with one to three substituents; and heteroaryl having 5 to 10 ring atoms and containing 1 to 3 heteroatoms each independently selected from N, O and S, the heteroaryl being optionally substituted with one to three substituents. By way of further example, R² may be selected from phenyl optionally substituted with one to three substituents selected from H, C₁ to C₆ alkyl, halo, C₁ to C₆ haloalkyl and C₁ to C₆ alkoxy; and heteroaryl having 5 to 6 ring atoms and containing 1 or 2 N atoms, the heteroaryl being optionally substituted with one to three substituents selected from C₁-C₆ alkyl (e.g. C₁ to C₃ alkyl), halo (e.g. F), C₁-C₆ haloalkyl (e.g. C₁ to C₃ haloalkyl) and C₁ to Ce alkoxy (e.g. C₁ to C₃ alkoxy). In some cases, suitable examples of R² include (but are not limited to) optionally substituted phenyl, and optionally substituted pyrazolyl.

Where R² is selected from optionally substituted heterocycloalkyl, the heterocycloalkyl may have 3 to 10 ring atoms and contain 1 to 3 heteroatoms each independently selected from N, O and S, and the heterocycloalkyl may be optionally substituted with one to three substituents. In some examples, the heterocycloalkyl may have 5 to 8 ring atoms (e.g. 6 ring atoms) and may contain 1 or 2 N atoms. In some cases, suitable examples include (but are not limited to) optionally substituted piperidinyl, and optionally substituted piperazinyl.

Further examples of suitable R² groups are shown below:

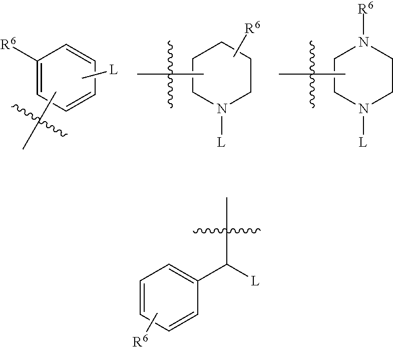

wherein in the structures shown above, R⁶ may be selected from H, C₁-C₆ alkyl, halo, C₁-C₆ haloalkyl and C₁-C₆ alkoxy. In some examples, R⁶ may be selected from H and C₁-C₆ alkyl.

In the structures shown above, the line intersected by a wavy line represents the covalent bond between the exemplary R² groups shown above and a carbon atom on the heterocycloalkyl core attached to the R² group in the parent structure of Z (as illustrated by the various formulae described herein). Although a particular substitution pattern is shown in the exemplary structures above, it will be appreciated that other substitution patterns are also encompassed within the scope of the present disclosure.

In addition, the bond to L shows the point of attachment to the linker. In the exemplary aryl structure above, it will be appreciated that the linker may replace a hydrogen atom at any suitable position on the aryl ring (e.g. provided it is chemically suitable and has the correct valency).

With respect to the various structures for Z defined by the formulae herein, R³ is selected from C₁ to C₆ alkyl, aryl, heteroaryl, substituted C₁ to C₆ alkyl, substituted aryl, and substituted heteroaryl.

In some examples, R³ may be selected from the group consisting of: C₁ to C₆ alkyl optionally substituted with a heterocycloalkyl group having 5 to 7 ring atoms and containing 1 or 2 heteroatoms each independently selected from N, O and S; aryl having 6 to 10 carbon ring atoms; and heteroaryl having 5 to 10 ring atoms and containing 1 to 3 heteroatoms each independently selected from N, O and S; wherein the aryl and the heteroaryl are optionally substituted with one or two substituents selected from the group consisting of halo, C₁ to C₃ alkyl, C₁ to C₃ haloalkyl and C₁ to C₃ alkoxy. By way of further example, in some cases the aryl and heteroaryl may be optionally substituted with one or two substituents selected from halo (e.g. F) and C₁ to C₃ alkyl (e.g. methyl).

Representative examples of suitable R³ groups include, but are not limited to, thiazolyl, pyridinyl, benzothiazolyl, phenyl, pyrazolyl, isoxazolyl, isothiazolyl, oxetanyl, cyclobutanyl, cyclopropanyl, tert-butyl, imidazolyl, oxazolyl, thiophenyl, imidazo(1,2-a)pyridinyl, N—C₁ to C₆ alkylenemorpholine, and 4,5,6,7-tetrahydro-1,3-benzothiazolyl, such as thiazolyl, pyridinyl, benzothiazolyl, phenyl, pyrazolyl, isoxazolyl, isothiazolyl, tetrahydropyranyl, tetrahydrofuranyl, oxetanyl, cyclobutanyl, cyclopropanyl and tert-butyl.

In each case, these R³ groups may be substituted, such as substituted thiazolyl, substituted pyridinyl, substituted benzothiazolyl, substituted phenyl, substituted pyrazolyl, substituted isoxazolyl, substituted isothiazolyl, substituted tetrahydropyranyl, substituted tetrahydrofuranyl, substituted oxetanyl, substituted cyclobutanyl, substituted cyclopropanyl and substituted tert-butyl. Where R³ is a substituted heteroaryl or aryl group, there may be one or more substituents on the aromatic ring e.g. it may be mono-, di- or tri-substituted. Where R³ is optionally substituted pyrazolyl or imidazolyl, a nitrogen atom of the pyrazolyl or imidazolyl ring may be substituted with C₁ to C₆ alkyl, such as methyl.

Representative examples of suitable R³ groups include, but are not limited to, optionally substituted phenyl, optionally substituted thiazolyl, optionally substituted pyrazolyl, optionally substituted oxazoyl, optionally substituted isoxazolyl, tert-butyl, C₁-C₆ alkyl comprising a morpholino substituent, optionally substituted benzothiazolyl and optionally substituted pyridinyl. Where R³ is a substituted aryl or heteroaryl group, there may be one or more substituents on the aromatic ring e.g. it may be mono-, di- or tri-substituted.

Further examples of suitable R³ groups are shown below:

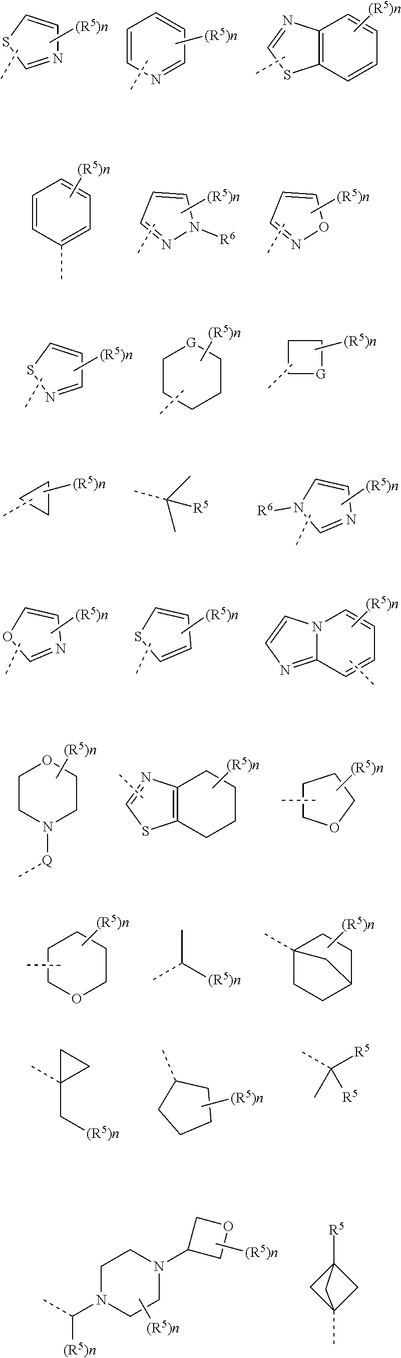

wherein the dotted line on the structures indicates the position that each of the respective R³ groups may be joined to the structure shown in the formulae described herein. Where the dotted line is not shown connected directly to an atom, the R³ group may be connected to the structure shown in formulae by a covalent bond to an atom at any position on the aromatic ring (provided that it has the correct valency and/or is chemically suitable). For example, a hydrogen at any position on the R³ group may be replaced with a bond to the parent structures as shown in the formulae described herein.

R⁵ may be any substituent as described herein or may be absent. In some examples, R⁵ may be selected from halo (e.g. F, Cl, Br, I), CF₃, —CH₂F, —CHF₂, OCF₃, —OCH₂F, —OCHF₂, C₁ to C₆ alkyl, —CN, —OH, —OMe, —SMe, —SOMe, —SO₂Me, —NH₂, —NHMe, —NMe₂, CO₂Me, —NO₂, CHO, and COMe. As stated above, there may be one or more substituents on the aromatic ring (e.g. n may be 0 to 5, such as 0 to 4, 0 to 3, or 0 to 2). Where more than one substituent is present, each substituent may be independently selected from the R⁵ groups noted above.

R⁶ may be C₁ to C₆ alkyl, such as methyl.

G may be selected from CH₂, O and NH.

Q may be C₁ to C₆ alkylene such as dimethylmethylene (—C(CH₃)₂—) or dimethylethylene (—C(CH₃)₂CH₂—).

In further embodiments, R³ is selected from the group consisting of:

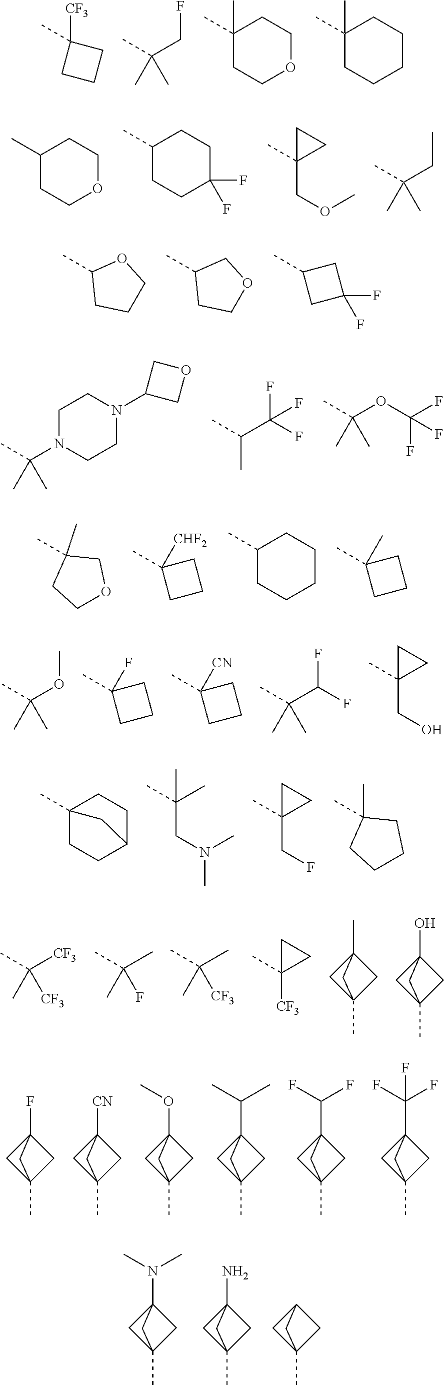

wherein the dotted line indicates the position at which each of the respective R³ groups is joined to the structure in the formulae described herein.

By way of further example, R⁵ may be selected from C₁ to C₆ alkyl (e.g. methyl) and halo (e.g. F). As stated above, there may be one or more substituents on the aromatic ring. Where two or more substituents are present, each substituent may be independently selected from the R⁵ groups noted above. Again, where present and unless otherwise indicated, R⁵ may be appended to the aryl or heteroaryl ring at any position (provided that it has the correct valency and/or is chemically suitable).

In the structures shown above, the line intersected by a wavy line represents the covalent bond between the exemplary R³ groups shown above and the carbon atom of the parent structure of Z (as illustrated by the various formulae described herein). In those cases where R³ is an aryl or heteroaryl group, this covalent bond (as illustrated in the various formulae described herein) may be formed at any position on the aromatic ring (provided that it has the correct valency and/or is chemically suitable). For example, a hydrogen at any position on the R³ groups shown above may be replaced with a bond to the structure shown in formula (I).

By way of further example, a suitable R³ group may be selected from the following:

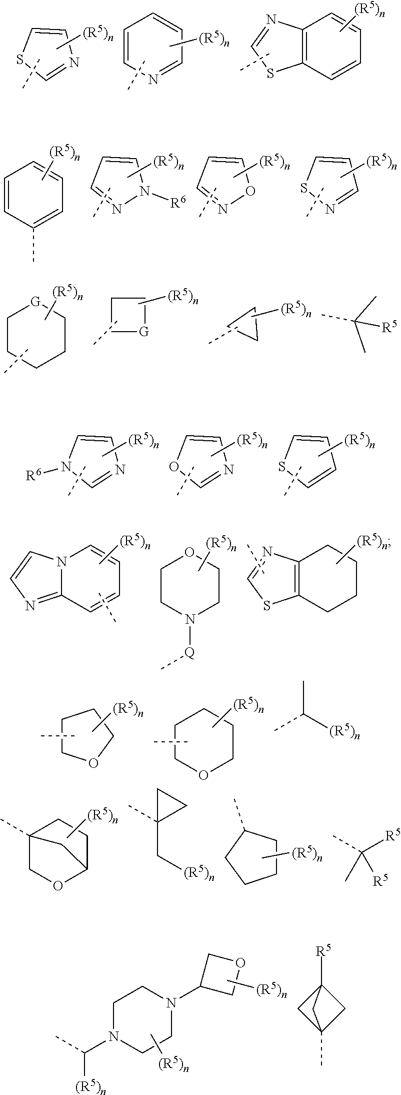

wherein the dotted line on the structures indicates the position that each of the respective R³ groups may be joined to the structure shown in formulae described herein, and R⁵, R⁶, n and G are as defined above.

In other examples, a suitable R³ group may be selected from the following:

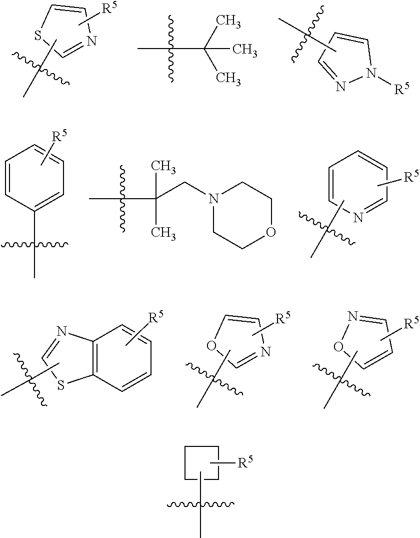

wherein the line intersected by a wavy line represents the covalent bond between the exemplary R³ groups shown above and the carbon atom of the parent structure of Z (as illustrated by the various formulae described herein), and R⁵ is as defined above.

By way of further example, a suitable R³ group may be selected from the following:

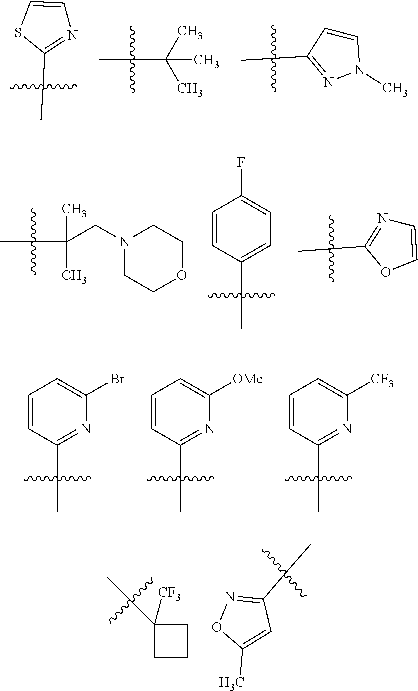

Again, in the structures shown above, the line intersected by a wavy line represents the covalent bond between the exemplary R³ groups shown above and the carbon atom of the parent structure of Z (as illustrated by the various formulae described herein).

As stated above, R⁴ may be selected from aryl, substituted aryl, heteroaryl and substituted heteroaryl. In some examples, R⁴ may be selected from aryl having 6 to 10 carbon ring atoms; and heteroaryl having 5 to 10 ring atoms and containing 1 to 3 heteroatoms each independently selected from N, O and S; wherein the aryl and the heteroaryl are optionally substituted with one or two substituents selected from the group consisting of halo, C₁ to C₃ alkyl, C₁ to C₃ haloalkyl and C₁ to C₃ alkoxy. In some examples, R⁴ may be an optionally substituted phenyl.

By way of further example, a suitable R⁴ group may be selected from the following:

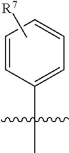

R⁷ may be any substituent as described herein or may be absent. In some examples, R⁷ may be selected from C₁ to C₆ alkyl, halo, C₁ to C₆ haloalkyl and C₁ to C₆ alkoxy. In some examples, R⁶ may be C₁ to C₆ alkyl or C₁ to C₃ alkyl (e.g. methyl). As stated above, there may be one or more substituents on the aromatic ring. Where two or more substituents are present, each substituent may be independently selected from the R⁷ groups noted above. Again, where present and unless otherwise indicated, R⁷ may be covalently bonded to the aryl or heteroaryl ring at any position (provided that it has the correct valency and/or is chemically suitable).

By way of further example, representative examples of Z are illustrated below:

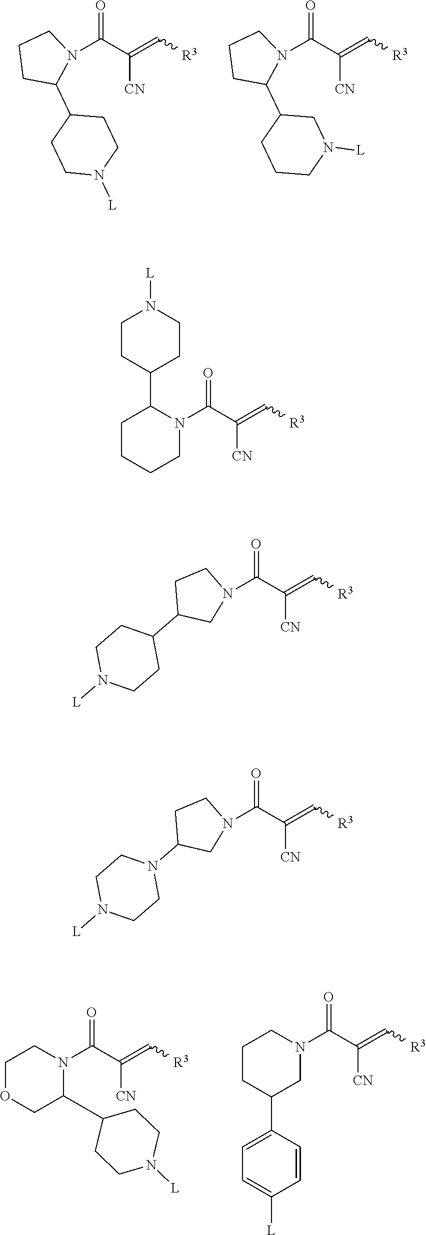

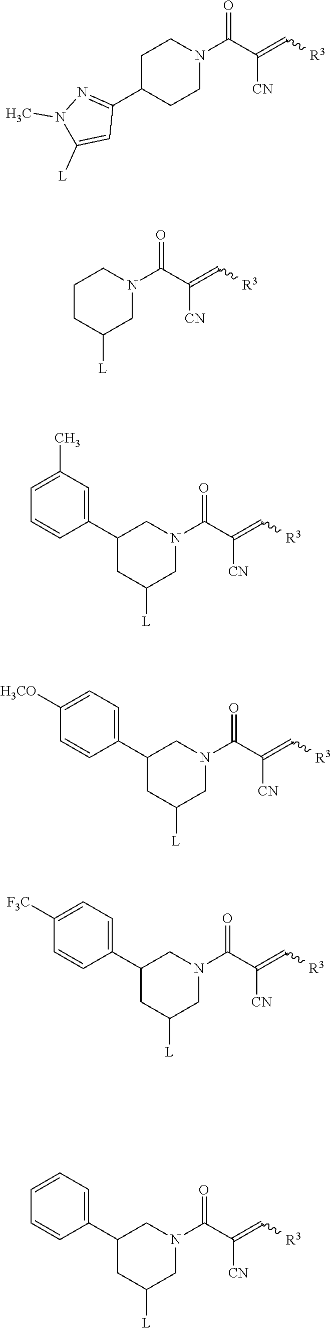

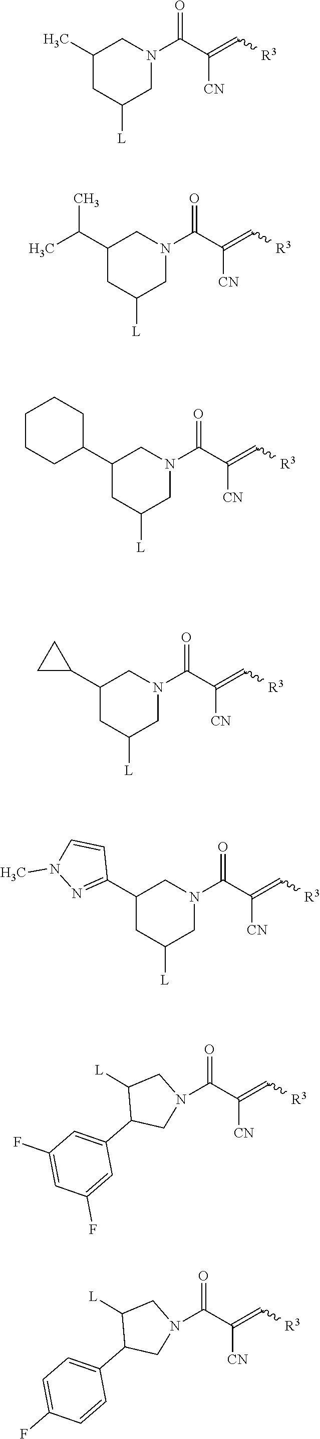

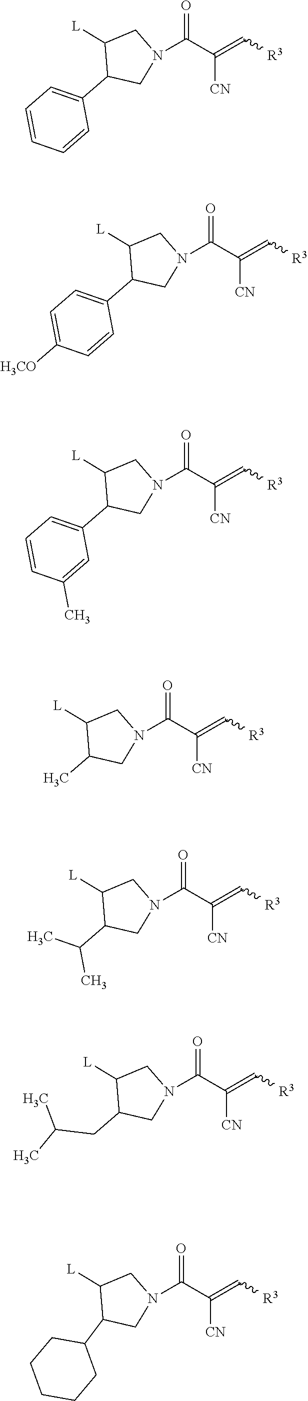

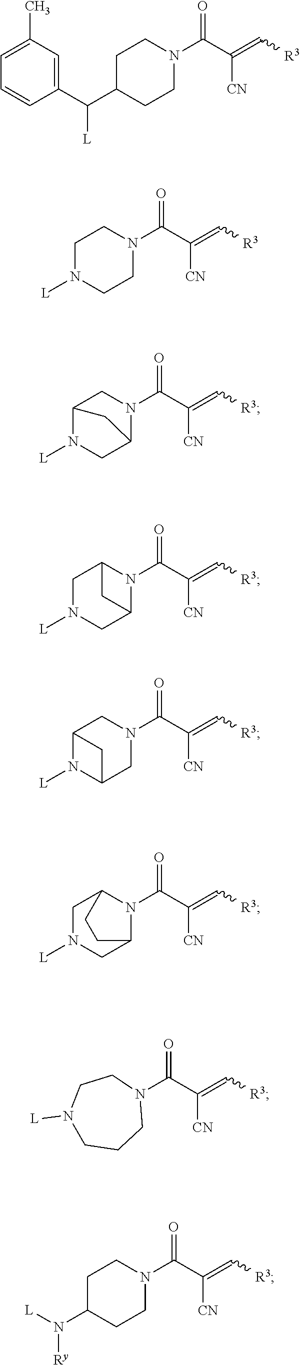

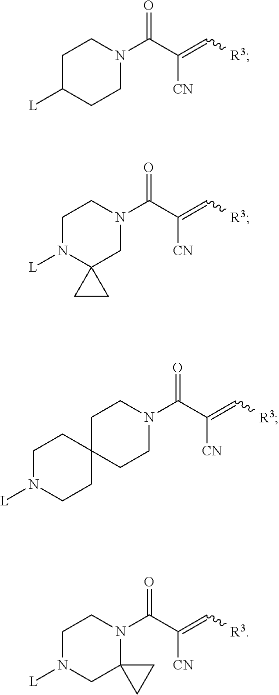

In the exemplary structures shown above, R³ may be selected from any of those R³ groups disclosed herein. In some cases, in the exemplary structures shown above, R³ may be selected from the group consisting of:

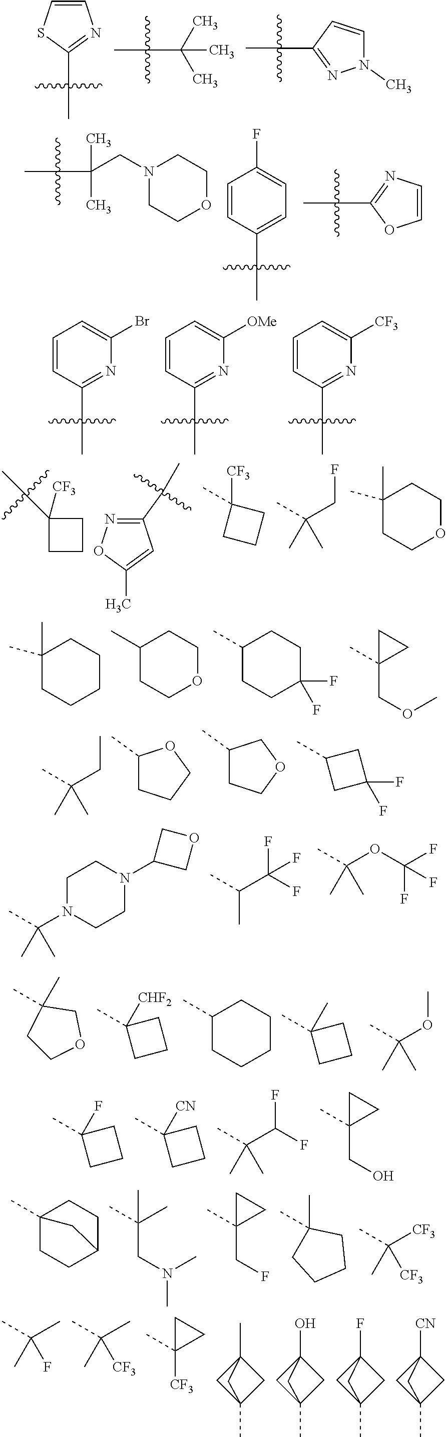

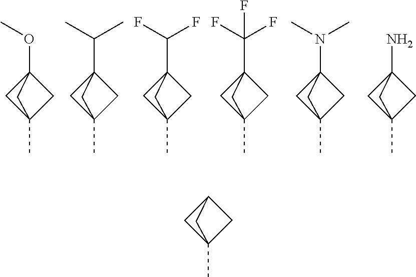

As noted above, Z is not:

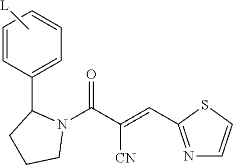

In some examples, Z is not (or does not comprise) a structure selected from one or more of the following:

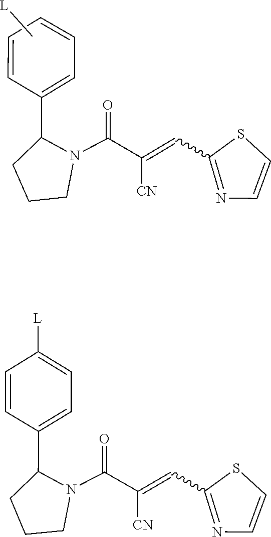

In some examples, Z is not (or does not comprise) the following structure:

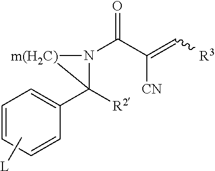

wherein R²′ is selected from H and C₁ to C₆ alkyl;
R³′ is selected from C₁ to C₆ alkyl, aryl, heteroaryl, substituted aryl, and substituted heteroaryl, optionally wherein the C₁ to C₆ alkyl is substituted with a heterocycloalkyl group;
m is 3, 4 or 5; and
L shows the point of attachment of the linker.

In some examples, the bifunctional molecule is not:

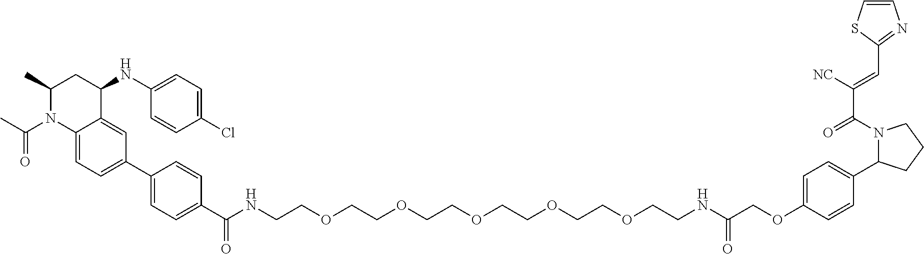 [SMILES: CC(=O)N1c2ccc(-c3ccc(C(=O)NCCOCCOCCOCCOCCOCCNC(=O)COc4ccc(C5CCCN5C(=O)/C(C#N)=C/c5nccs5)cc4)cc3)cc2[C@H](Nc2ccc(Cl)cc2)C[C@@H]1C]

Alternatively it is noted, that whilst the various formulae described herein indicate that the linker is joined to the Z moiety via the heterocyclic core (either directly or indirectly via the R² group), the present disclosure also extends to examples wherein the linker is attached at any other position in the Z moiety (provided that it has the correct valency and/or is chemically suitable). For example, the linker may replace a hydrogen atom at any position in the Z moiety. Thus, in some examples, Z may be represented as shown in formula (ZV) or (V):

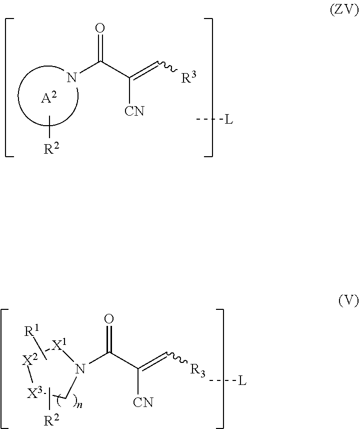

wherein ring A², R¹, R², R³, X¹, X², X³, n and L are as defined in any one of the embodiments disclosed above.

The dotted line shown through the square brackets on formulae (ZV) and (V) indicates that the linker may be joined via a covalent bond to any atom on the Z moiety provided that it has the correct valency, is chemically suitable and/or provided that the attachment of the linker at this alternative position does not disrupt the function of the Z moiety in promoting and/or facilitating proteasomal degradation.

According to a further aspect of the disclosure, there are also provided compounds comprising a general structure of:

L-Z
wherein moiety Z is as defined in any one of the formulae described herein (e.g. any one of formulae (I) to (V) and (ZI) to (ZV); and
L is a linker as defined herein.

Such compounds may be useful in a synthesis of the described bifunctional molecules, e.g. via a modular approach, wherein each of moieties TBL, Z and L are provided as separate building blocks. In some examples, L and Z may be joined to provide the compounds L-Z as described above (which may then be further reacted to join to an appropriate TBL moiety).

### Intermediate/Fragment

As described above, structures comprising a Z moiety as described herein may find particular utility in targeted protein degradation. As such, intermediates comprising such Z moieties may have value in providing useful intermediates for the synthesis of bifunctional molecules for use in targeted protein degradation.

According to a further aspect, there is provided an intermediate compound or fragment corresponding to any one or more of formulae for Z described herein or any of the other example structures described herein, in which the L-W or W moiety has been replaced with a group “G”.

G is attached to the disclosed structures by way of a covalent bond

According to a further aspect, there is provided a compound comprising the Z moiety according to formula (VI) or (VIa):

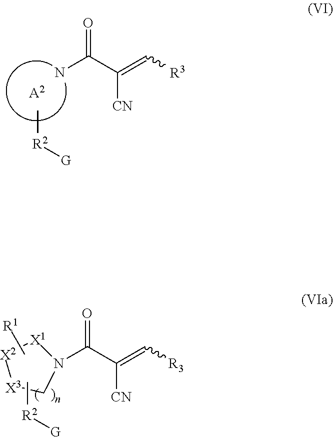

wherein ring A², R¹, R², R³, n, X¹, X² and X³ are as defined in any one of the embodiments disclosed above.

As shown in formula (VI) and (VIa), G is appended to the heterocyclic core either directly or via the R² group. In either case, G is attached to moiety Z by way of a covalent bond. G may be attached (either directly or indirectly via R²) at any position on the heterocyclic core (provided it has the correct valency and/or is chemically suitable). For example, G may replace a hydrogen atom at any position on the heterocyclic core.

The group G in formula (VI) or (VIa) is configured to enable attachment of the Z moiety to another chemical structure (such as a linker moiety or a linker-target protein binding ligand moiety) via formation of a new covalent bond. Following the formation of this new covalent bond, the group G may form part of the linker as defined herein.

In some examples, G may comprise a functional group that is able to facilitate the formation of a new covalent bond between Z and another moiety, e.g. via formation of an amide, ester, thioester, keto, urethane, amine, or ether linkage, or via formation of a new carbon-carbon bond or new carbon-nitrogen bond.

By way of example only, G may be represented as shown below:

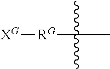

wherein R^{G} is absent or is a C₁ to C₆ alkyl, optionally substituted with one or more heteroatoms selected from N, O and S;
X^{G} is a group that is selected from —CO₂H, —(CO)—N-hydroxysuccinimide and —(CO)-pentafluorphenol esters, —CHO, —COR^{G1}, —OH, —NH₂, —NHR^{G2}, halo (e.g. iodo and bromo), O— leaving group (such as —OTs (tosylate), OMs (mesylate), —OTf (triflate)), alkynyl, azide, dienyl, aminoxy, tetrazinyl, (E)-cyclooctenyl, cyclooctynyl, norbornyl, boronic acid, boronate ester, alkylboranes or an organometallic group (e.g. organotin, zinc or other suitable reagent); and
R^{G1} and R^{G2} are each independently selected from C₁ to C₆ alkyl.

In this structure, a wavy line is shown over the bond that forms the link with the heterocyclic core of Z (or the R² group if present).

G is linked to the heterocyclic core shown in formula (VI) (either directly or via the R² group) by way of the R^{G} group. In those cases where R^{G} is absent, the group X^{G} is directly attached to the heterocyclic core or R² shown in formula (VI).

Representative examples of suitable G moieties are shown below:

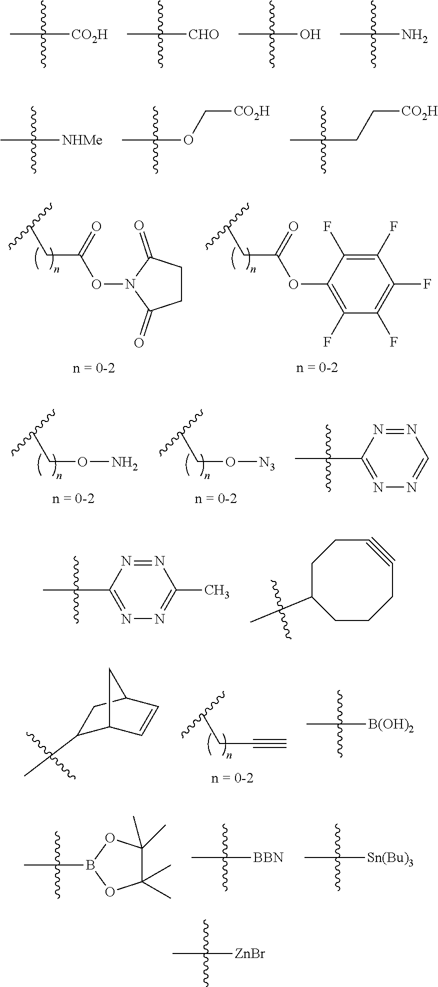

It is noted that the disclosure further extends to any of the structures for Z shown in the formulae defined herein, such as formulae (I) to (V) and (ZI) to (ZV) or other representative examples of Z, wherein the group L on these structures has been replaced with the group G as defined above in respect of formula (VI) or (VIa).

### Linker (L)

As described herein, the TBL is linked or coupled to moiety Z via a linker L. The linker may be a chemical linker (e.g. a chemical linker moiety) and, for example, may be a covalent linker, by which is meant that the linker is coupled to Z and/or TBL by a covalent bond.

The linker acts to tether the target protein binding ligand and Z moieties to one another whilst also allowing both of these portions to bind to their respect targets and/or perform their intended function. In particular, the linker may act to tether the target protein binding ligand to Z whilst also mitigating the possibility of the Z moiety disrupting, interfering with and/or inhibiting the binding of the target protein binding ligand to the target protein. Additionally or alternatively, the linker may act to tether Z to the target protein binding ligand whilst also mitigating the possibility of the target protein binding ligand disrupting, interfering with and/or inhibiting the cellular interactions of Z (e.g. its function in modulating, facilitating and/or promoting the proteasomal degradation of the target protein).

In other words, the linker may function to facilitate targeted protein degradation by allowing each end of the bifunctional molecule to be available for binding (or another type of interaction) with various components of the cellular environment. For example, the linker may be configured to allow the target protein binding ligand to bind to the target protein without interference, disruption and/or inhibition from the Z moiety of the bifunctional molecule. Additionally or alternatively, the linker may be configured to allow the Z moiety to interact with the various components in the cellular environment to modulate, facilitate and/or promote the proteasomal degradation of the target protein without interference, disruption and/or inhibition from the target protein binding ligand of the bifunctional molecule.

In many cases, a broad range of linkers will be tolerated. The selection of linker may depend upon the protein being targeted for degradation (the target protein) and/or the particular target protein binding ligand.

The linker may be selected to provide a particular length and/or flexibility, e.g. such that the target protein binding ligand and the Z moiety are held within a particular distance and/or geometry. As will be appreciated by one of skill in the art, the length and/or flexibility of the linker may be varied dependent upon the structure and/or nature of the target protein binding ligand.

In some examples, the TBL is connected directly to moiety Z by a covalent bond i.e, the linker is a covalent bond. Such a direct connection is also encompassed within the term “linker” within the context of the present disclosure (and unless otherwise stated).

By way of example only, the linker may comprise any number of atoms between 1 and 200, between 1 and 100, between 1 and 50, between 1 and 30 or between 1 and 10. In some cases the linker may comprise any number of atoms in a single linear chain of between 1 and 200, between 1 and 100, between 1 and 50, between 1 and 30 or between 1 and 10. In some examples of the disclosure, the linker may comprise any number of atoms in a single linear chain between 1 and 25, such as 25, or between 1 and 20, such as 3 and 20, or between 1 and 18, such as 3 and 18.

The degree of flexibility of the linker may depend upon the number of rotatable bonds present in the linker. A rotatable bond is defined as a single non-ring bond, bound to a nonterminal heavy atom (e.g. non-hydrogen atom). As described herein, an amide (C—N) bond is not considered rotatable because of the high rotational energy barrier. In some cases, the linkers may comprise one or more moieties selected from rings, double bonds and amides to reduce the flexibility of the linker. In other cases, the linker may comprise a greater number and/or proportion of single bonds (e.g. may predominantly comprise single non-ring bonds) to increase the flexibility of the linker. It may also be appreciated that the length of the linker may affect the degree of flexibility. For example, a shorter linker comprising fewer bonds may also reduce the flexibility of a linker.

In some examples, the number of rotatable bonds present in the linker may be any number between 1 and 20, between 1 and 15, or between 1 and 10. In some examples, the number of rotatable bonds present in the linker may be any number between 2 and 9, between 2 and 8, or between 3 and 6.

In some examples, the linker may comprise any number of atoms in a single linear chain between 10 and 20; and/or the number of rotatable bonds present in the linker may be any number between 2 and 8.

The structure of the linker (L) may be represented as follows:

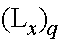

wherein each Lₓ represents a subunit of L; and
q is an integer greater than or equal to 1.

For example, q may be any integer between 1 and 30, between 1 and 20 or between 1 and 5.

By way of example, in the case where q is 1, the linker comprises only one Lₓ subunit and may be represented as L₁. In the case where q is 2, the linker comprises two Lₓ subunits that are covalently linked to one another and which may be represented as L₁-L₂. In another example, where q is 3, the linker comprises three Lₓ subunits that are covalently linked to one another and may be represented as L₁-L₂-L₃. For even higher integer values of q, L may comprise the following subunits L₁, L₂, L₃, L₄ . . . up to L_{q}.

Each of Lₓ may be independently selected from CR^{L1}R^{L2}, O, C═O, S, S═O, SO₂, NR^{L3}, SONR^{L4}, SONR^{L5}C═O, CONR^{L6}, NR^{L7}CO, C(R^{L8})═C(R^{L9}), C═C, aryl, substituted aryl, heteroaryl, substituted heteroaryl, cycloalkyl, substituted cycloalkyl, heterocycloalkyl and substituted heterocycloalkyl groups.

Each of R^{L1}, R^{L2}, R^{L3}, R^{L4}, R^{L5}, R^{L6}, R^{L7}, R^{L8} and R^{L9} may be independently selected from H, halo, C₁ to C₆ alkyl, C₁ to C₆, haloalkyl, —OH, —O(C₁ to C₆ alkyl), —NH₂, —NH(C₁ to C₆ alkyl), —NO₂, —CN, —CONH₂, —CONH(C₁ to C₆ alkyl), —CON(C₁ to C₆ alkyl)₂, —S(O)OC₁ to C₆ alkyl, —C(O)OC₁ to C₆ alkyl, and —CO(C₁ to C₆ alkyl). In some examples, each of R^{L1}, R^{L2}, R^{L3}, R^{L4} R^{L5}, R^{L6}, R^{L7}, R^{L8} and R^{L9} may be independently selected from H and C₁ to C₆ alkyl.

The terms aryl, substituted aryl, heteroaryl, substituted heteroaryl, cycloalkyl, substituted cycloalkyl, heterocycloalkyl and substituted heterocycloalkyl groups are defined above.

The terminal Lₓ subunits may link or couple the linker moiety to the TBL and Z moieties of the bifunctional molecule. For example, if the terminal Lₓ subunits are designated as L₁ and L_{q}, L₁ may link the linker to the TBL moiety and L_{q} may link the linker to the Z moiety. In those cases where q is 1, the one Lₓ subunit (e.g. L₁) provides the link between the TBL and Z moieties of the bifunctional molecule.

The TBL and Z moieties may be covalently linked to L through any group which is appropriate and stable to the chemistry of the linker. By way of example only, the linker may be covalently bonded to the TBL moiety via a carbon-carbon bond, keto, amino, amide, ester or ether linkage. Similarly, the linker may be covalently bonded to the Z moiety via a carbon-carbon bond, carbon-nitrogen bond, keto, amino, amide, ester or ether linkage.

In some cases, each terminal Lₓ subunit (e.g. L₁ and L_{q}) is independently selected from O, C═O, CR^{L1}R^{L2}, NR^{L3}, CONR^{L6}, NR^{L7}CO, aryl, substituted aryl, heteroaryl, substituted heteroaryl, cycloalkyl, substituted cycloalkyl, heterocycloalkyl and substituted heterocycloalkyl groups.

In some examples, at least one of Lₓ comprises a ring structure and is, for example, selected from a heterocycloalkyl, heteroaryl, cycloalkyl or aryl group.

In alternative examples, the linker may be or comprise an alkyl linker comprising, a repeating subunit of —CH₂—; where the number of repeats is from 1 to 50, for example, 1-50, 1-40, 1-30, 1-20, 1-19, 1-18, 1-17, 1-16, 1-15, 1-14, 1-13, 1-12, 1-11, 1-10, 1-9. 1-8, 1-7, 1-6, 1-5, 1-4, 1-3 and 1-2.

In other examples, the linker may be or comprise a polyalkylene glycol. By way of example only, the linker may be or comprise a polyethylene glycol (PEG) comprising repeating subunits of ethylene glycol (C₂H₄O), for example, having from about 1-50 ethylene glycol subunits, for example where the number of repeats is from 1 to 100, for example, 1-50, 1-40, 1-30, 1-20, 1-19 1-18, 1-17, 1-16, 1-15, 1-14, 1-13, 1-12 or 1-5 repeats.

In some of the examples described herein, the structure of the linker (L) may be, or comprise, a structure represented as shown in formula (L1a):

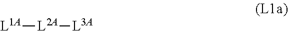

wherein L^{1A} is absent or is selected from C₁-C₆ alkylene (e.g. ethylene), C₁-C₆ alkoxy (e.g. —O(CH₂)—, —O(CH₂)₂—, —O(CH₂)₅—, —CH₂OCH₂—) and C₁-C₆ alkylamino (e.g. —NR^{L2A}(CH₂)—, —R^{L2A} (CH₂)₂—, —R^{L2A} (CH₂)₅—, —CH₂R^{L2A}CH₂—);
L^{2A} is —NR^{L2A}C═O— or —C═ONR^{L2A}—; and
L_{3A} is selected from C₁-C₃ alkylene (e.g. ethylene), C₁-C₆ alkoxy (e.g. —(CH₂)O—, —(CH₂)₂O—, —(CH₂)₅O—, —CH₂OCH₂—) and C₁-C₆ alkylamino (e.g. —(CH₂) NR^{L2A}—, —(CH₂)₂NR^{L2A}—, —(CH₂)₅NR^{L2A}—, —CH₂NR^{L2A}CH₂—);
   wherein R^{L2A} is H or C₁-C₆ alkyl (e.g. C₁-C₃ alkyl).

In further examples, the structure of the linker (L) may be, or comprise, a structure represented as shown in formula (L₁b):

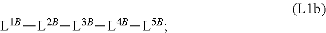

wherein L^{1B} is absent or is selected from C₁-C₃ alkylene (e.g. ethylene), C₁-C₆ alkoxy (e.g. —O(CH₂)—, —O(CH₂)₂—, —O(CH₂)₅—, —CH₂OCH₂—) and C₁-C₆ alkylamino (e.g. —NR^{L2A}CH₂)—, —NR^{L2A}CH₂)₂—, —R^{L2A}CH₂)₅—, —CH₂R^{L2A}CH₂—);
L^{2B} is —NR^{L2A}C═O— or —C═ONR^{L2A}—;
L^{3B} is selected from C₁-C₁₅ alkylene, —[(CH₂)₂O]₁₋₆(CH₂)₂—;
L^{4B} is —NR^{L2A}C═O— or —C═ONR^{L2A}— wherein R^{L2A} is H or C₁-C₆ alkyl (e.g. C₁-C₃ alkyl);
L^{5B} is selected from C₁-C₃ alkylene (e.g. ethylene), C₁-C₆ alkoxy (e.g. —(CH₂)O—, —(CH₂)₂O—, —(CH₂)₅O—, —CH₂OCH₂—) and C₁-C₆ alkylamino (e.g. —(CH₂) NR^{L2A}—, —NR^{L2A}CH₂)₂—, —(CH₂)₅NR^{L2A}—, —CH₂NR^{L2A}CH₂—);
   wherein R^{L2A} is H or C₁-C₆ alkyl (e.g. C₁-C₃ alkyl).

In some of the examples described herein, the structure of the linker (L) may be, or comprise, a structure represented as shown in formula (L1c):

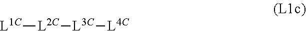

wherein L^{1C} is an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S;
L^{2C} is absent or is selected from C₁-C₃ alkylene (e.g. ethylene), C₁-C₆ alkoxy (e.g. —(CH₂)O—, —(CH₂)₂O—, —(CH₂)₅O—, —CH₂OCH₂—) and C₁-C₆ alkylamino (e.g. —(CH₂) NR^{L2A}—, —(CH₂)₂NR^{L2A}—, —(CH₂)₅NR^{L2A}—, —CH₂NR^{L2A}CH₂—);
L₃C is —R^{L2B}C═O— or —(C═O)R^{L2}B—; and
L_{4C} is selected from C₁-C₃ alkylene (e.g. ethylene), C₁-C₆ alkoxy (e.g. —(CH₂)O—, —(CH₂)₂O—, —(CH₂)₅O—, —CH₂OCH₂—) and C₁-C₆ alkylamino (e.g. —(CH₂) NR^{L2A}—, —(CH₂)₂NR^{L2A}—, —(CH₂)₅NR^{L2A}—, —CH₂NR^{L2A}CH₂—);
   wherein:
   R^{L2A} is H or C₁-C₆ alkyl (e.g. C₁-C₃ alkyl); and
   R^{L2B} is NR^{L2A}; or an N-linked optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S.

In examples of Linker (L) represented by the Formula L1c, L^{1C} and L^{2C} may be both absent. In such examples, R^{L2B} in L^{3C} is an N-linked optionally substituted 4- to 7-membered monocyclic N-heterocycloalkyl, optionally containing one or two additional ring heteroatoms selected from N, O and S, and L^{3C} is the terminal subunit of the linker attached, suitably covalently attached, to the TBL via R^{L2B}.

In some of the examples described herein, the structure of the linker (L) may be, or comprise, a structure represented as shown in formula (L1d):

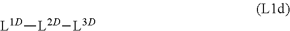

wherein L^{1D} is absent or is selected from C₁-C₃ alkylene, CO, C₁-C₃ alkylene (N(C₁-C₃ alkyl);
L^{2D} is NR^{L2A} or an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from N, O and S; wherein R^{L2A} is H or C₁-C₆ alkyl (e.g. C₁-C₃ alkyl); and
L^{3D} is absent or is selected from C₁-C₃ alkylene, —O—, —N(C₁-C₃ alkyl)-, and CO.

In further examples, the structure of the linker (L) may be, or comprise, a structure represented as shown in formula (L1e):

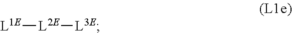

wherein L^{1E} is C₁-C₃ alkylene (e.g. methylene) or CO;
L^{2E} is an optionally substituted 4- to 7-membered monocyclic N-heterocycloalkyl, an optionally substituted 7- to 12-membered bicyclic N-heterocycloalkyl, each optionally containing one or two additional ring heteroatoms selected from N, O and S; and
L^{3E} is selected from C₁-C₃ alkylene (e.g. methylene).

In some examples, L^{1A}, L_{1B}, L^{1C}, L^{1D}, or L^{1E} is the terminal subunit of the linker structure attached (i.e. covalently bonded) to the W moiety and L^{3A}, L^{5B}, L^{4C}, L^{3D}, L^{3E}, is the terminal subunit of the linker structure attached (i.e. covalently bonded) to the TBL portion.

Where any of L^{1A}, L^{1B} or L^{1D} are absent, L^{2A}, L^{2B} or L^{2D} is directly attached (i.e. covalently bonded) to the W moiety. Where L^{3D} is absent, L^{2D} is directly attached (i.e. covalently bonded) to the TBL portion.

As stated above, a number of linker portions, such as L^{1C}, L^{2D}, L^{2E} examples of R^{L2B} and, may be bicyclic or tricyclic, and unless otherwise stated, these moieties may comprise rings that are joined by a bond, rings that are fused, a bridged ring and/or rings that are joined at a spiro centre.

When any one of L^{1C}, L^{2D}, L^{2E} examples of R^{L2B} is bicyclic, it may be a bridged bicyclic ring (i.e. it may comprise two rings that share three or more atoms) or it may be a spirocyclic bicyclic ring (i.e. it may comprise two rings that share one atom, e.g. the two rings may be joined at a spiro centre).

When any one of L_{1C}, L_{2D}, L^{2E} examples of R^{L2B} is a bridged bicyclic ring, it may be an optionally substituted 7- to 12-membered bridged bicyclic N-heterocycloalkyl optionally containing one or two additional ring heteroatoms selected from N, O and S. In some examples, L_{1C}, L_{2D}, L_{2E}, and examples of R^{L2B} may be a 7- or 8-membered bridged bicyclic N-heterocycloalkyl optionally containing one or two additional ring heteroatoms selected from N, O and S. In some examples, L_{1C}, L_{2D}, L_{2E}, and examples of R^{L2B} may be a 7- or 8-membered bridged bicyclic N-heterocycloalkyl optionally containing one additional ring atom selected from N.

When any one of L_{1C}, L_{2D}, L_{2E}, and examples of R^{L2B} is a spirocyclic bicyclic ring, it may be an optionally substituted 7- to 12-membered spirocyclic bicyclic N-heterocycloalkyl optionally containing one or two additional ring heteroatoms selected from N, O and S. In some examples, L_{1C}, L_{2D}, L_{2E}, and examples of R^{L2B} may be a 7- to 12-membered spirocyclic bicyclic N-heterocycloalkyl optionally containing one or two additional ring heteroatoms selected from N, O and S. In some cases, L_{1C}, L_{2D}, L_{2E}, and examples of R^{L2B} may be bicyclic and comprises a first 5- to 7-membered ring and a second 3- to 7-membered ring. For example, L_{1C}, L_{2D}, L_{2E}, and examples of R^{L2B} may be a spirocyclic bicyclic N-heterocycloalkyl comprising a first 5- or 6-membered ring and a second 3- to 6-membered ring, and optionally containing one or two additional ring heteroatoms selected from N, O and S. In some examples, L_{1C}, L_{2D}, L_{2E}, and examples of R^{L2B} may be a spirocyclic bicyclic N-heterocycloalkyl comprising a first 5- or 6-membered ring and a second 3- to 6-membered ring, and optionally containing one additional ring heteroatoms selected from N.

In some examples, the structure of L_{1C}, L_{2D}, L_{2E}, and examples of R^{L2B} may be any one selected from:

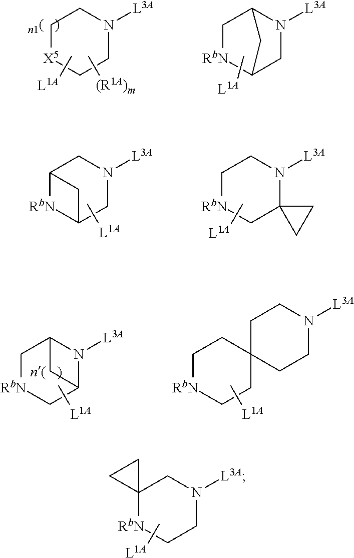

Wherein L^{1A} and L_{3A} are as defined above;
X⁵ is C(R^{b})₂, NR^{b} or O;
R^{b} is H or optionally substituted C₁₋₃alkyl;
n1 is 0, 1, 2 or 3;
n′ is 1 or 2;
m is 0, 1 or 2

The dotted line on the structures above indicates that the linker may be joined to the structure shown at any position indicated (provided that it has the correct valency and/or is chemically suitable).

In some examples L_{1C}, L_{2D}, L_{2E}, and examples of R^{L2B} is any one selected from:

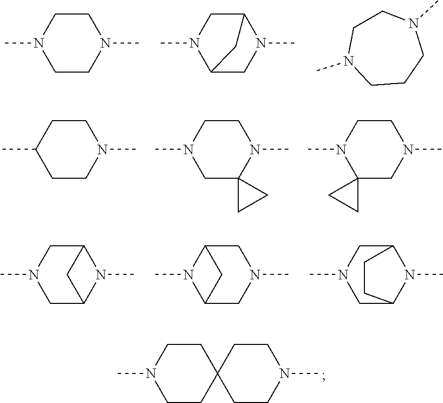

The dotted line on the structures above indicates that the linker may be joined to the structure shown at any position indicated (provided that it has the correct valency and/or is chemically suitable).

As stated above, L_{1D} is absent or is selected from C₁-C₃ alkylene, —O—, —N(C₁-C₃ alkyl)-, and CO. In some examples, L_{3D} is selected from C₁-C₃ alkylene (e.g. methylene).

In some of the examples described herein, the linker (L) may be, or comprise, a structure represented as shown in formula (L₁f):

L^{1F} (L₁f)
wherein L^{1F} is selected from C₁-C₃ alkylene, CO, and C₁-C₃ alkylene (NR^{L1C}); wherein R^{L1C} is H or C₁-C₃ alkyl.

In some examples, L^{1F} is selected from C₁-C₃ alkylene (such as methylene).

In any of the examples described herein, the linker is or comprises one or more of:

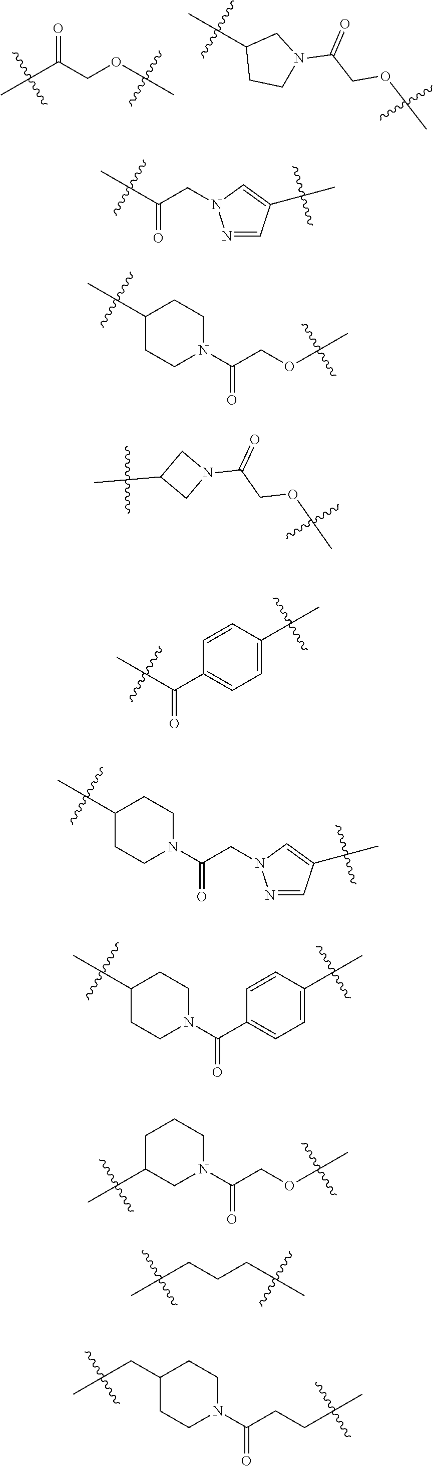

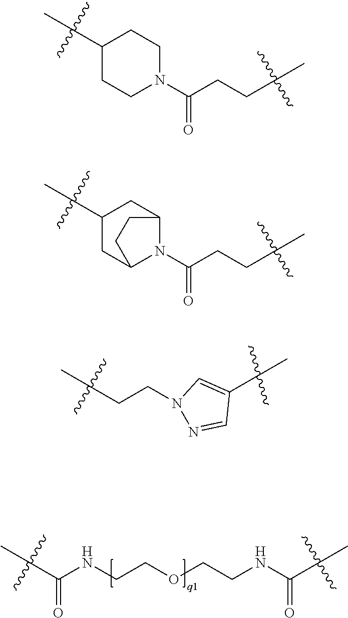

wherein q1 is any integer between 1 and 20, or between 1 and 10 (e.g. between 1 and 5). Alternatively, in any of the examples described herein, the linker is or comprises one or more of:

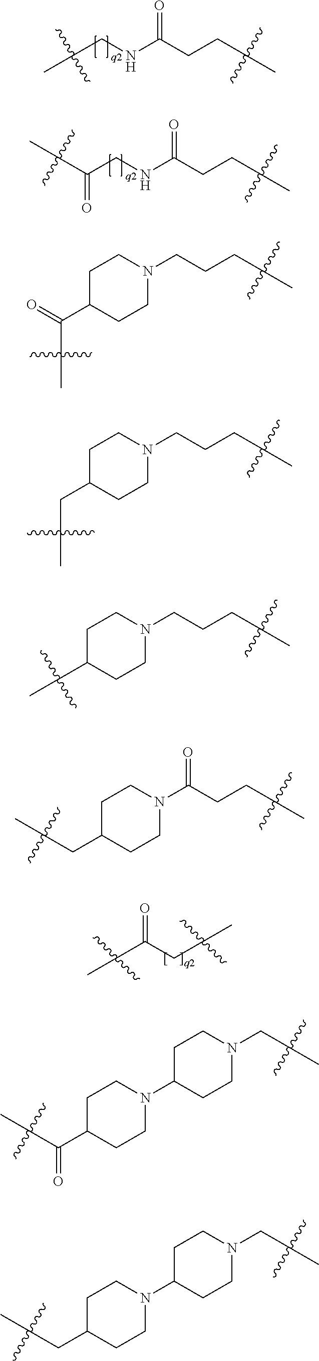

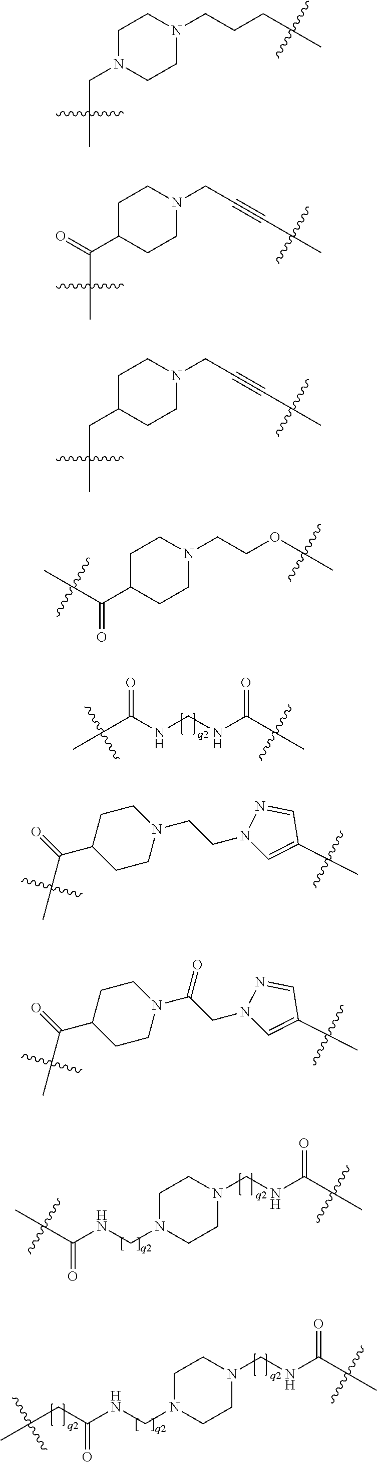

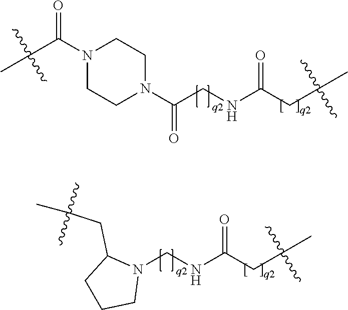

wherein q2 is any integer between 1 and 20, or between 1 and 10 (e.g. 3, 4, 5, 6 or 10).

As a further alternative, in any of the examples described herein, the linker is or comprises one or more of:

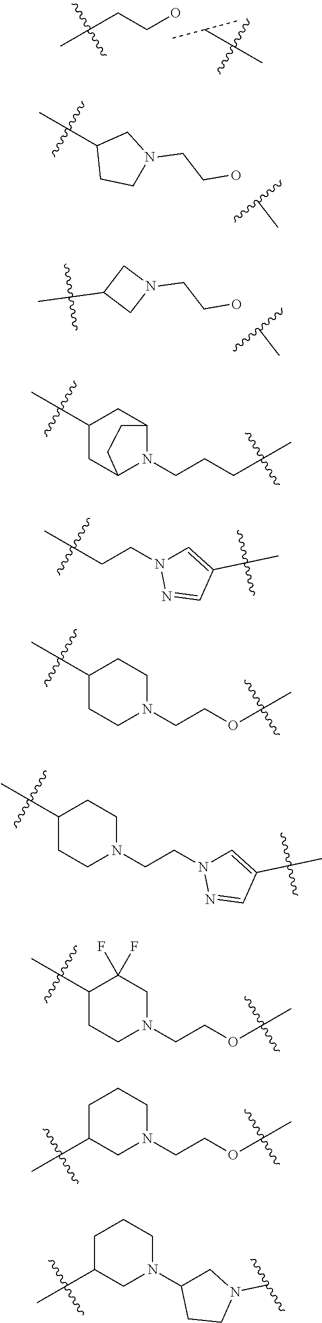

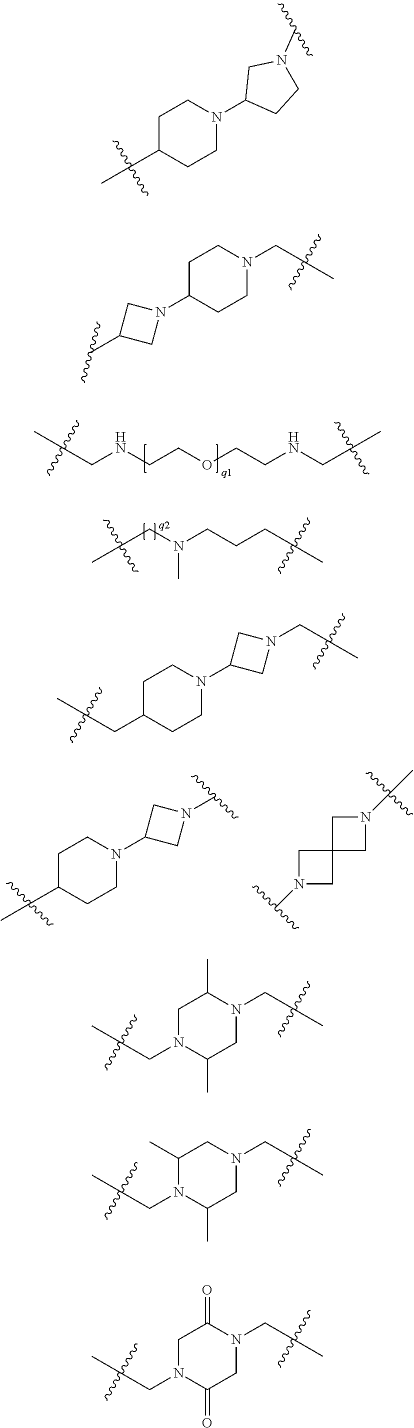

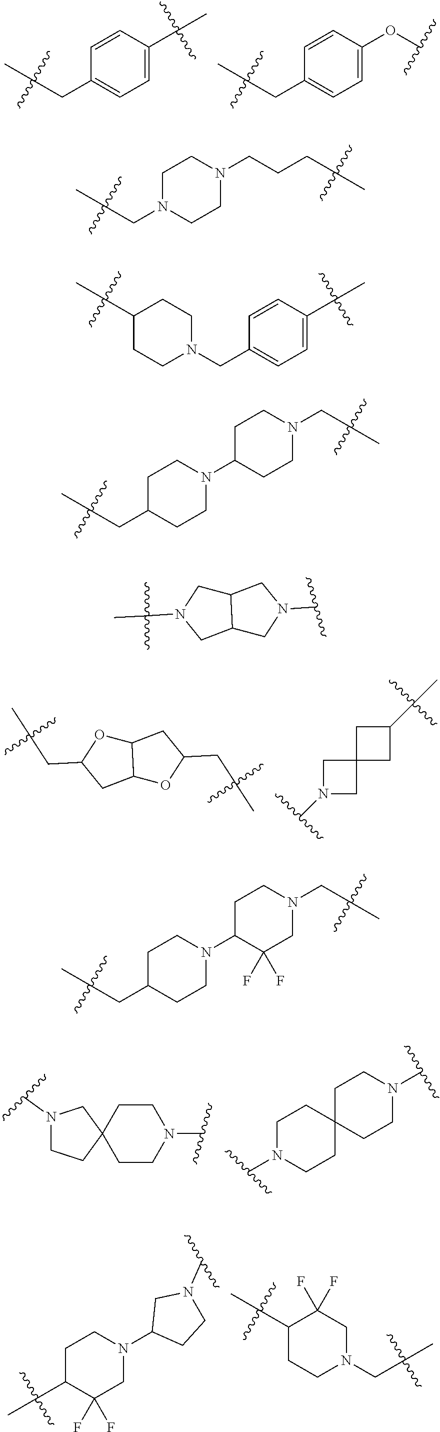

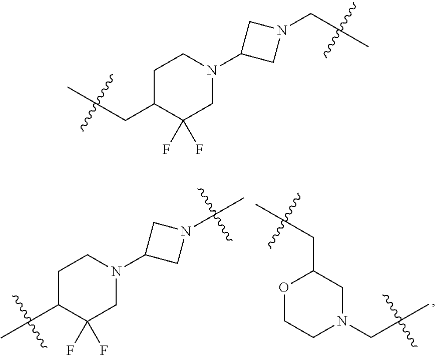

wherein q1 is any integer between 1 and 20, or between 1 and 10 (e.g. between 1 and 5) and
q2 is any integer between 1 and 20, or between 1 and 10 (e.g. 3, 4, 5, 6 or 10).

In particular examples, the linker is or comprises one or more of the following structures:

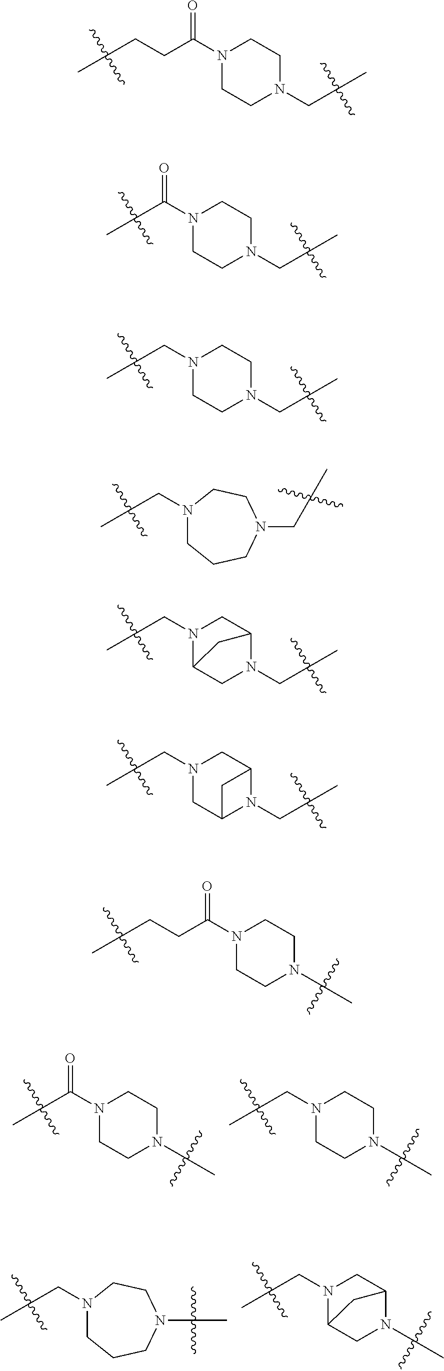

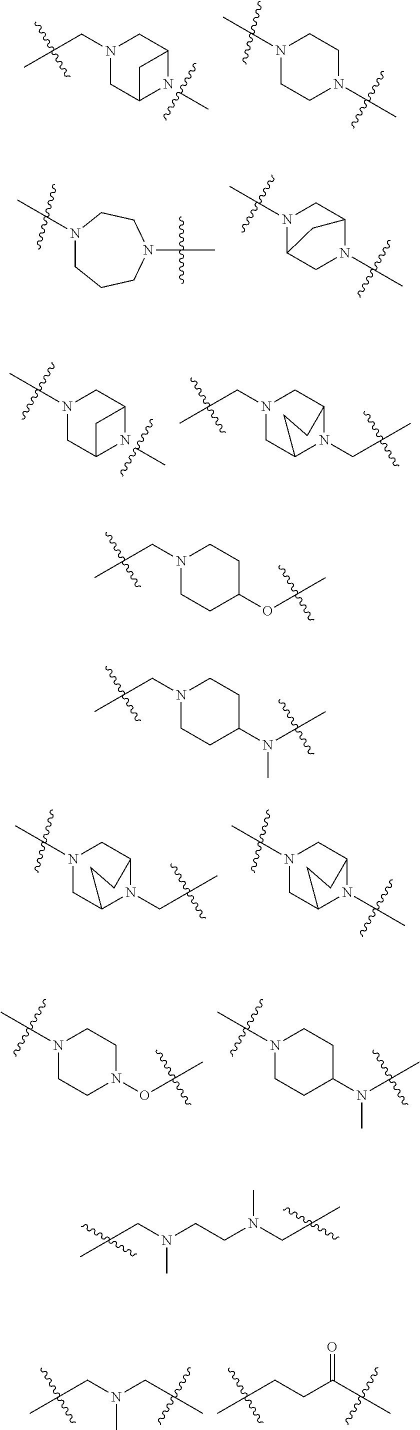

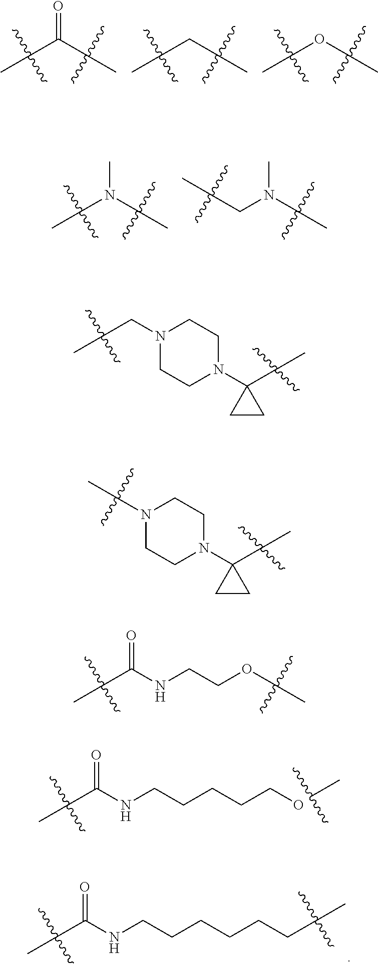

In yet further alternatives, in any of the examples described herein, the linker is or comprises one or more of:

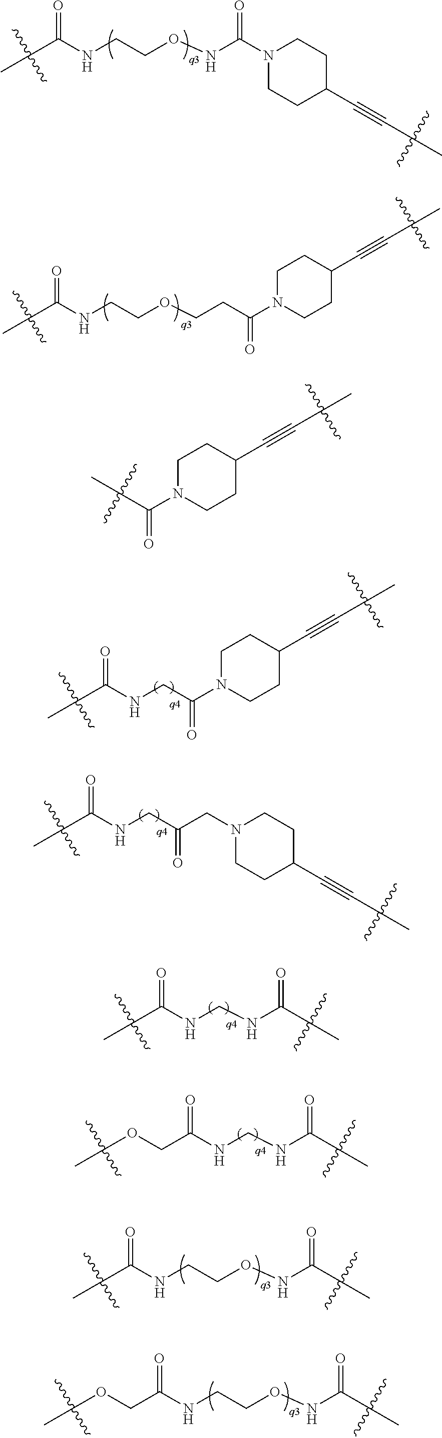

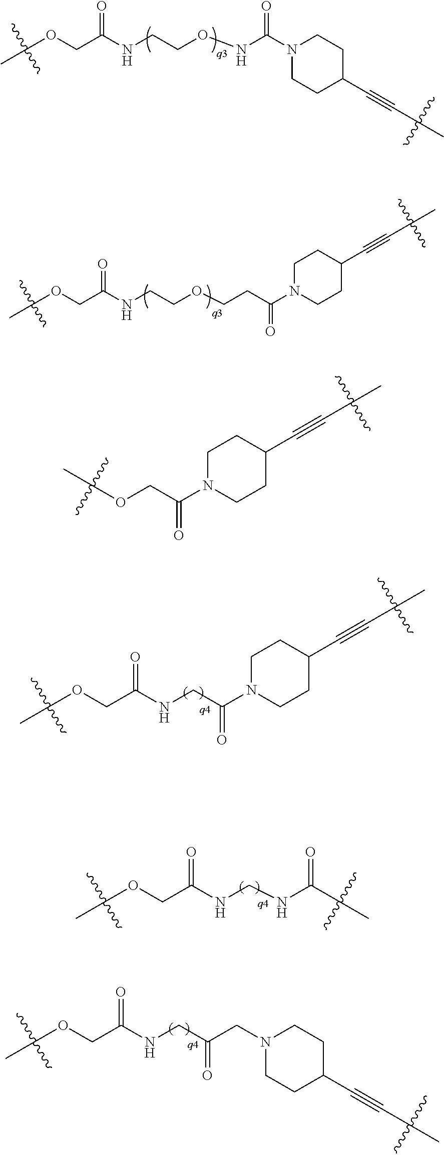

wherein q3 is 1 to 8, such as 1 to 5, and q4 is 1 to 12, such as 1 to 10.

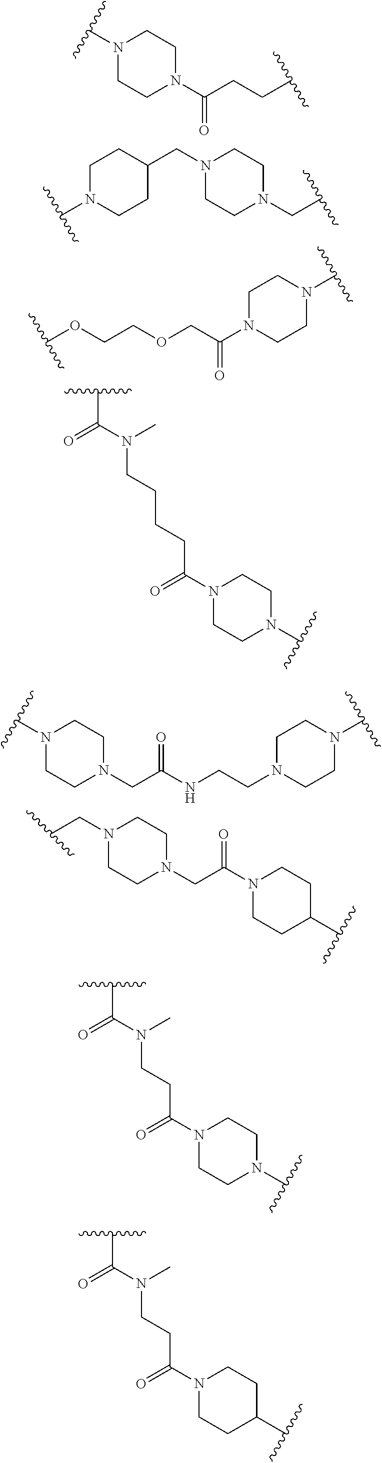

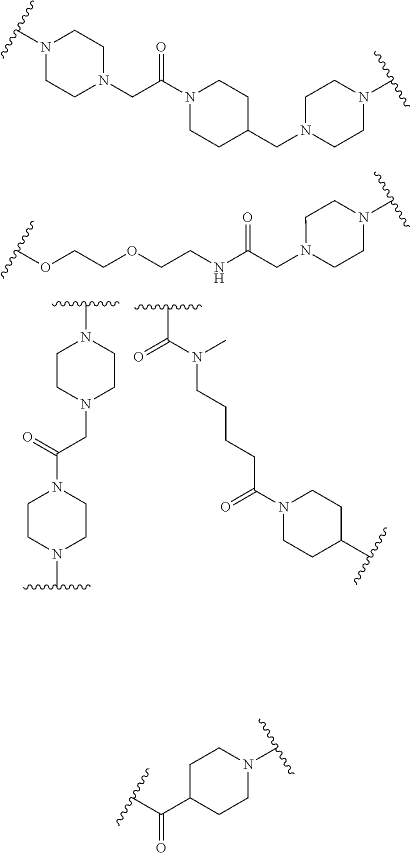

In particular examples, the linker is or comprises one or more of the following structures:

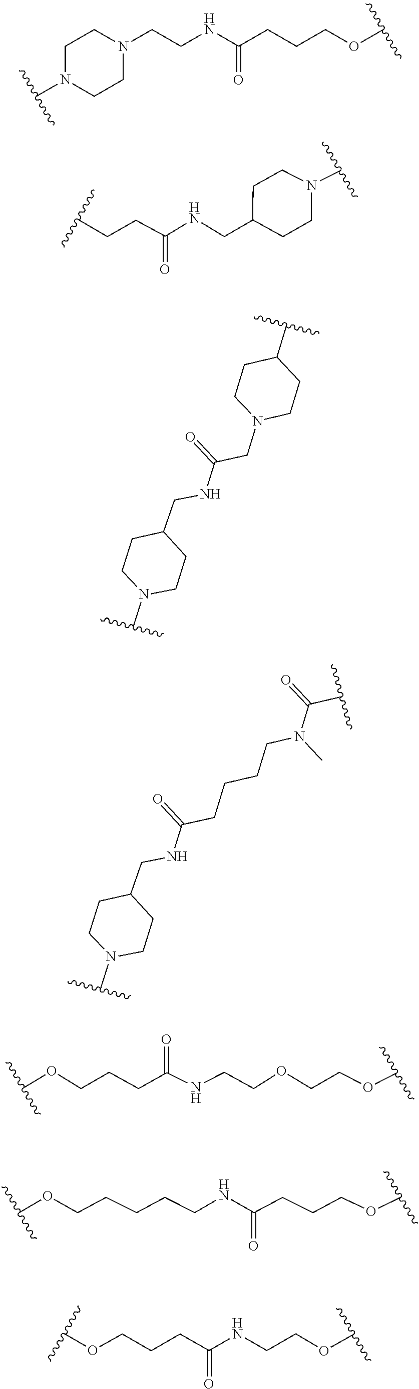

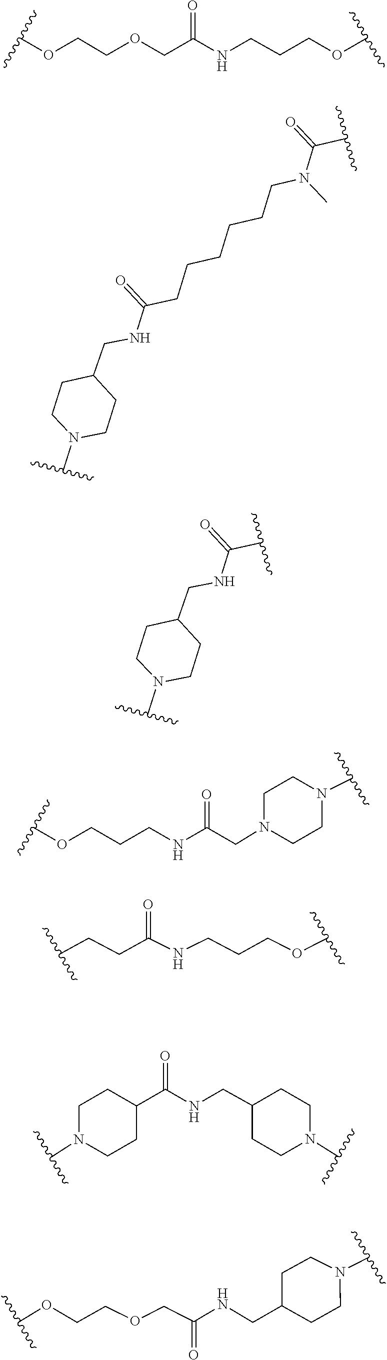

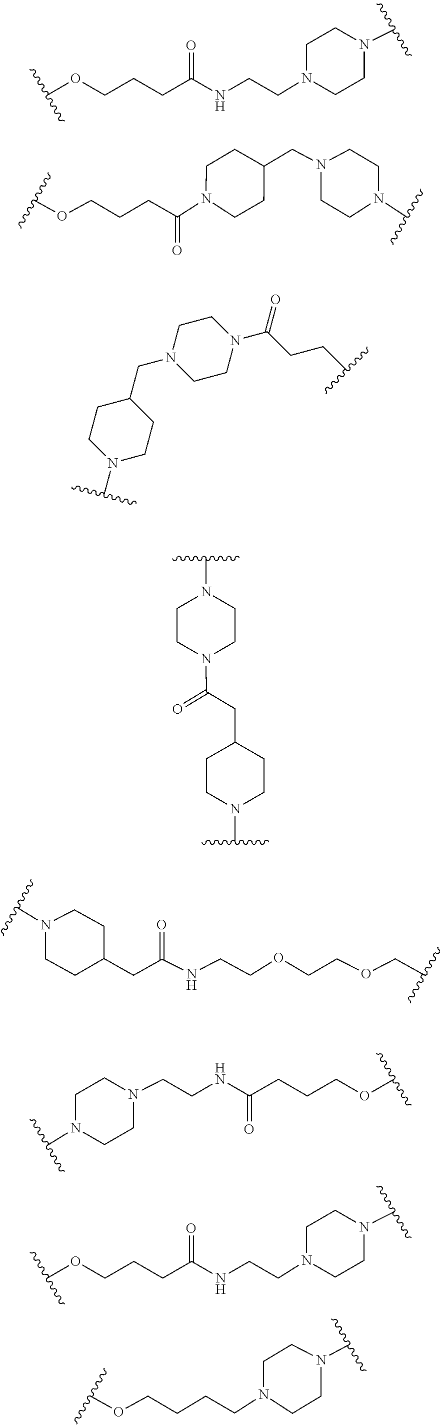

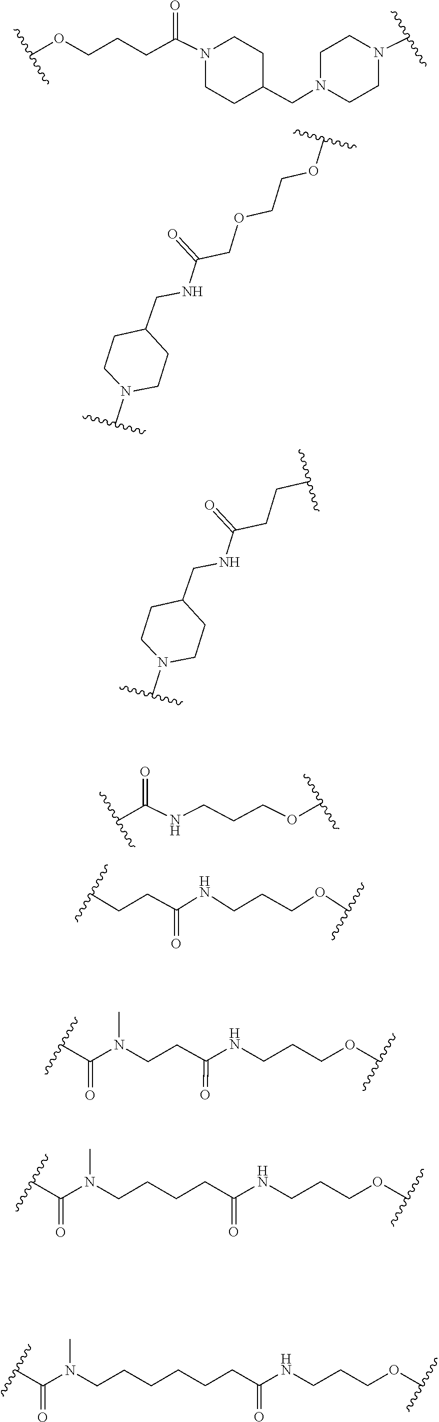

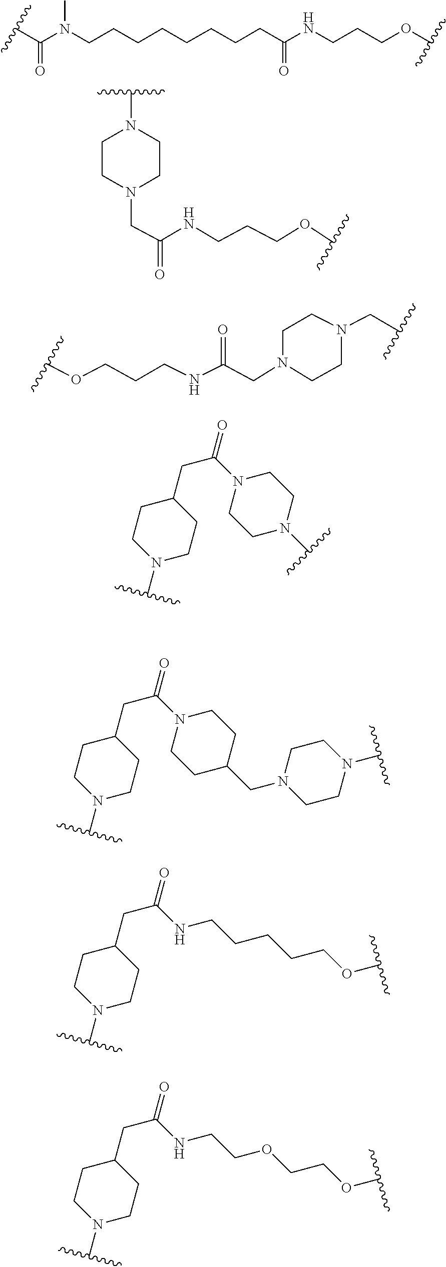

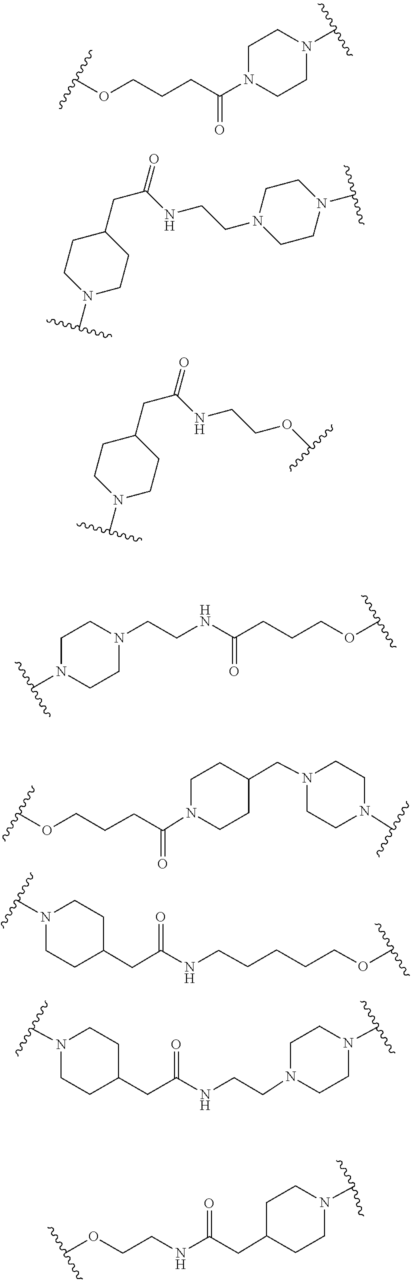

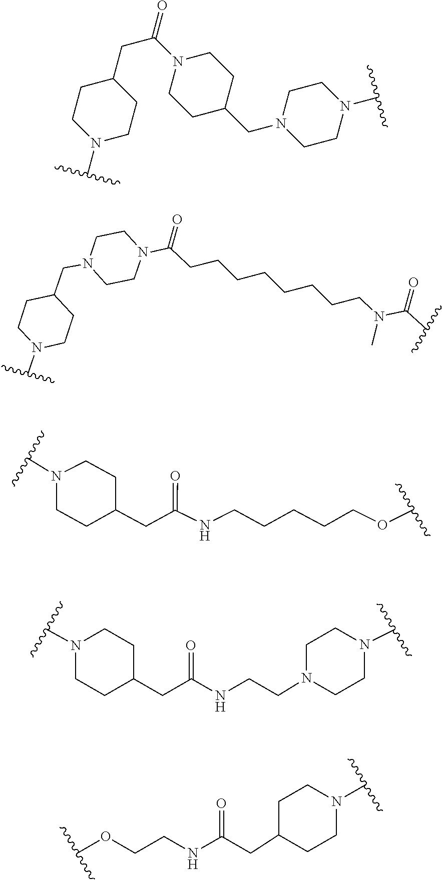 [SMILES: *N1CCC(CC(=O)N2CCC(CN3CCN(*)CC3)CC2)CC1 ; *C(=O)N(C)CCCCCCCCC(=O)N1CCN(CC2CCN(*)CC2)CC1 ; *OCCCCCNC(=O)CC1CCN(*)CC1 ; *N1CCC(CC(=O)NCCN2CCN(*)CC2)CC1 ; *OCCNC(=O)CC1CCN(*)CC1]

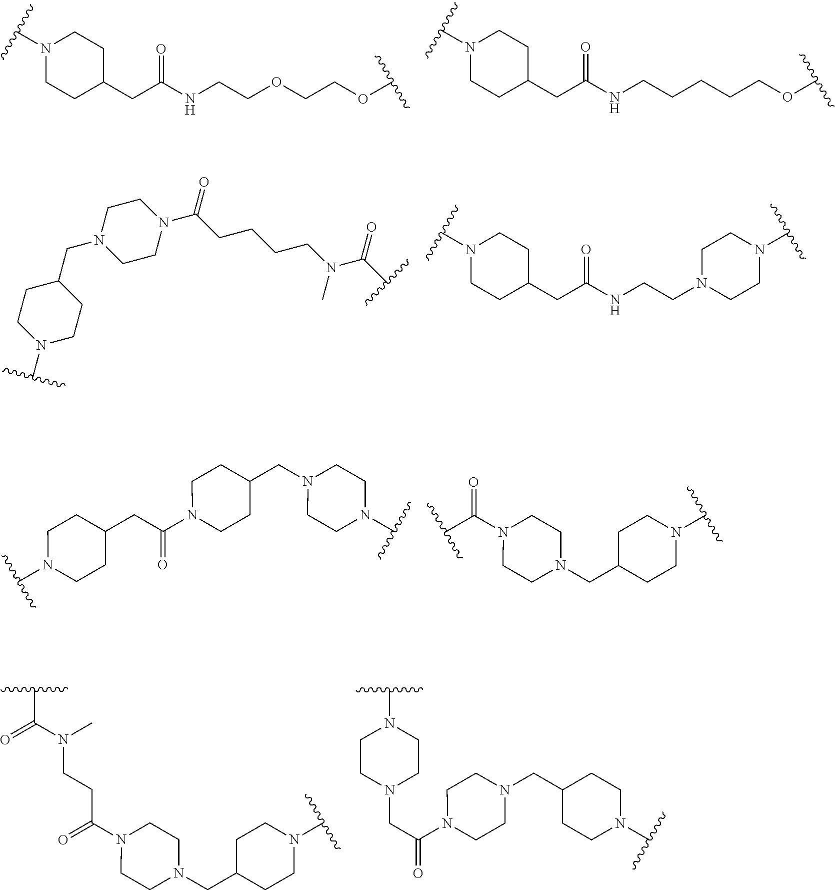

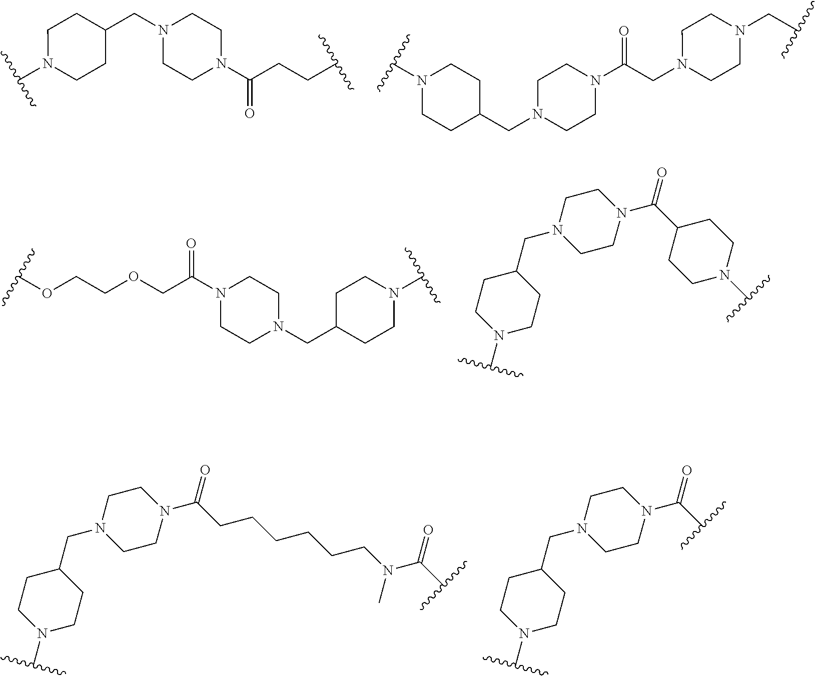 [SMILES: *CCC(=O)N1CCN(CC2CCN(*)CC2)CC1 ; *CN1CCN(CC(=O)N2CCN(CC3CCN(*)CC3)CC2)CC1 ; *OCCOCC(=O)N1CCN(CC2CCN(*)CC2)CC1 ; *N1CCC(CN2CCN(C(=O)C3CCN(*)CC3)CC2)CC1 ; *C(=O)N(C)CCCCCCC(=O)N1CCN(CC2CCN(*)CC2)CC1 ; *C(=O)N1CCN(CC2CCN(*)CC2)CC1]

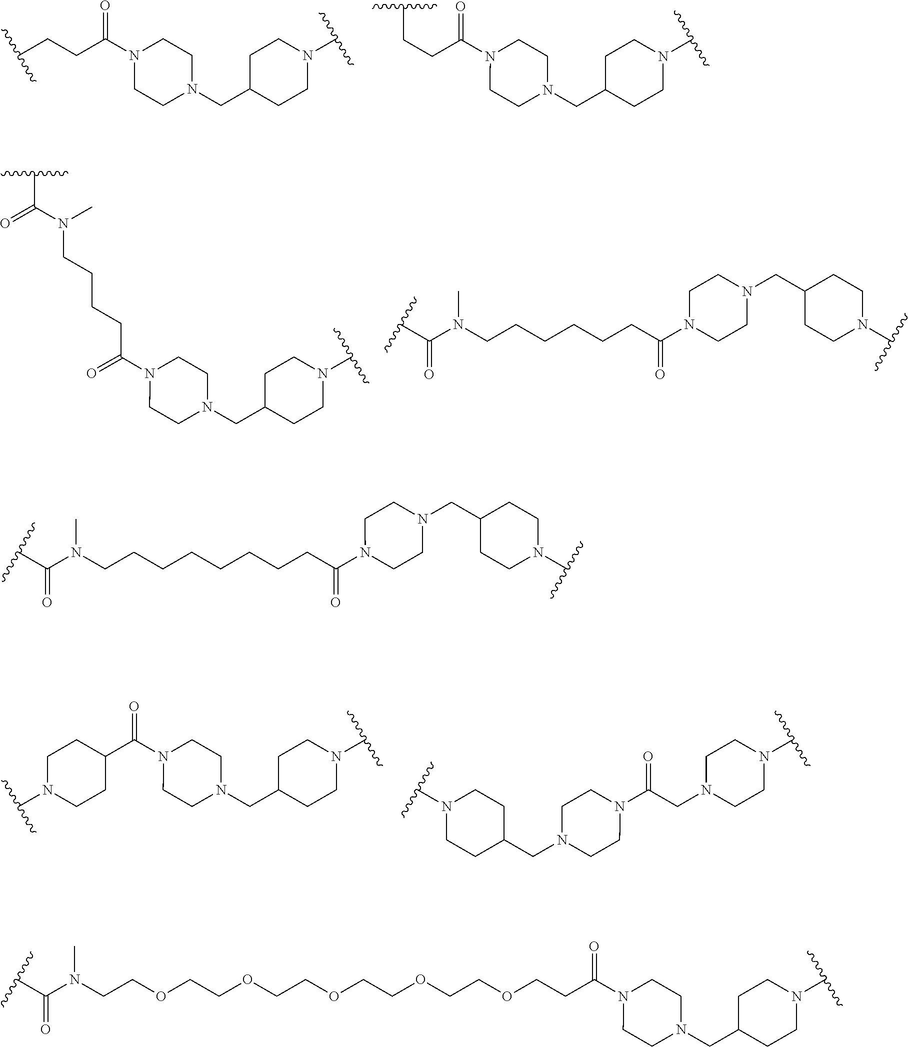

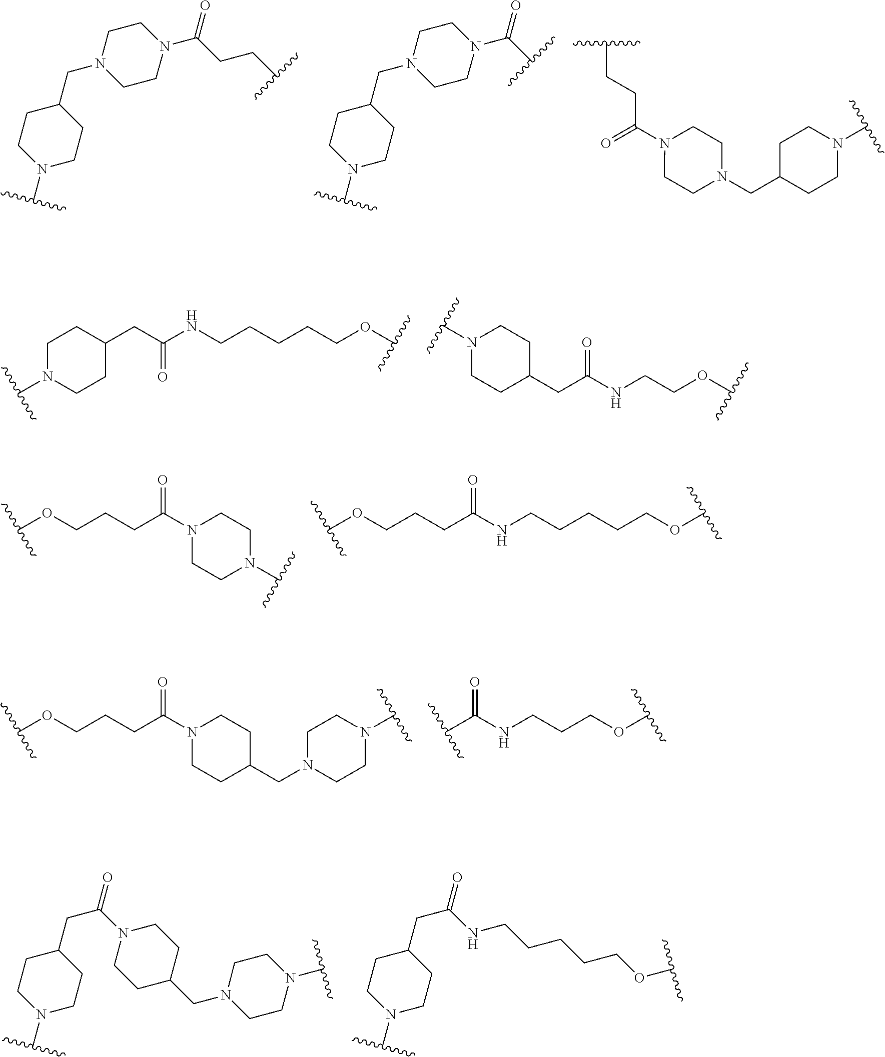

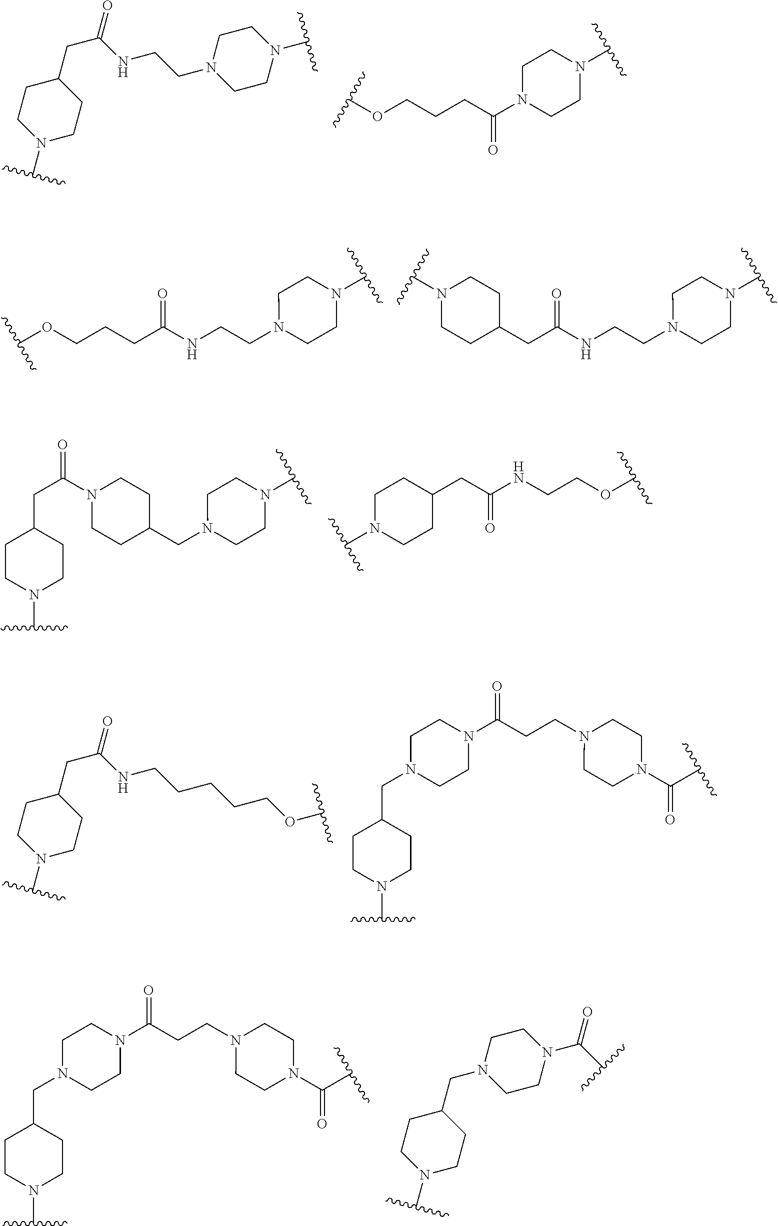

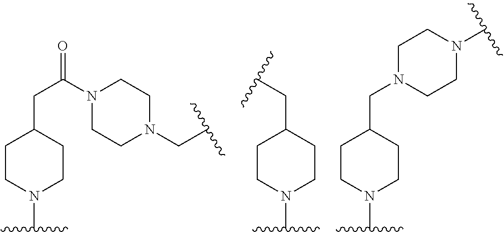

In some cases, the structures shown above represent the entire linker. In other examples, the linker of the bifunctional molecule may comprise a plurality of the structures shown above.

In these structures, the wavy lines are shown over the bond(s) that forms the link with the TBL and Z moieties respectively.

In some examples, the bond(s) that forms the link with the TBL and/or Z moieties is (are) attached to a ring structure. On many of the structures described herein, this bond is shown as being attached at a particular position on the ring structure. However, the disclosure also encompasses joining or coupling to the TBL and Z moieties at any chemically suitable position on these ring structures.

The present disclosure encompasses the use of any of the linkers disclosed herein in combination with any of the Z moieties and TBL moieties described herein.

### Target Protein

As used herein, a “target protein” may be any polypeptide or protein that the skilled practitioner wishes to selectively degrade in a cell or a mammal, e.g., a human or animal subject. In other words, a “target protein” may be a protein or polypeptide that is selected by the skilled practitioner for increased proteolysis in a cell. The term “selected target protein” may be any polypeptide or protein which has been selected to be targeted for protein degradation and/or increased proteolysis.

In some examples of the present disclosure, the term “target protein” does not include androgen receptor. As used herein, “androgen receptor” means a protein with the UniProtKB designation of P10275 (ANDR_HUMAN).

In some examples of the present disclosure, the term “target protein” does not include estrogen receptor. As used herein, “estrogen receptor” means a protein with the UniProtKB designation of P03372 (ESR1_HUMAN).

In other words, in some examples, the bifunctional molecules disclosed herein may not be intended for use or may not be suitable for use in the targeted degradation of a target protein selected from an: (i) estrogen receptor; and (ii) androgen receptor.

According to the disclosure, degradation of a target protein may occur when the target protein is subjected to and/or contacted with a bifunctional molecule as described herein, e.g. when the target protein is subjected to and/or contacted with any one of the bifunctional molecules in a cell.

Selective degradation and/or increased proteolysis of the target protein will reduce levels of the target protein and so can reduce the effects of the target protein in the cell. The control of specific protein levels afforded by the bifunctional molecules described herein may provide treatment of a disease state or condition, which is modulated through or by the target protein by lowering the level of that protein in the cells of a subject.

Target proteins that may be subject to increased proteolysis and/or selective degradation when contacted to the bifunctional molecules of this disclosure (and the associated methods of using such molecules) include any proteins and polypeptides. Target proteins include proteins and polypeptides having a biological function or activity such as structural, regulatory, hormonal, enzymatic, genetic, immunological, contractile, storage, transportation, and signal transduction functions and activities.

By way of example, target proteins may include structural proteins, receptors, enzymes, cell surface proteins, proteins pertinent to the integrated function of a cell, including proteins involved in catalytic activity, epigenetic regulation, aromatase activity, motor activity, helicase activity, metabolic processes (anabolism and catabolismantioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioural proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, and translation regulator activity.

Target proteins may include proteins from eukaryotes and prokaryotes, including humans, other animals, including domesticated animals, microbes, viruses, fungi and parasites, among numerous other targets for drug therapy.

In some examples, target proteins may include, but are not limited to: (i) kinases (such as serine/threonine kinases and receptor tyrosine kinases); (ii) bromodomain-containing proteins (such as BET family proteins); (iii) epigenetic proteins (including histone or DNA methyl transferases, acetyl transferases, deacetylases and demethylases); (iv) transcription factors (including STAT3 and myc); (v) GTPases (including KRAS, NRAS, and HRAS); (vi) phosphatases; (vii) ubiquitin E3 ligases and deubiquitinase enzymes; (viii) nuclear hormone receptors (including, for example, thyroid hormone receptor, androgen receptor (AR) and estrogen receptor (ER), but as stated above, in some examples, the target protein does not include androgen receptor and estrogen receptor); (ix) aggregation-prone proteins (including Beta-amyloid, tau, Htt, alpha-synuclein and polyQ-expanded proteins); (x) apoptotic & anti-apoptotic factors (including Bcl2, Bcl-xl and Mcl-1); and (xi) polymerases (including PARP & POLQ) among numerous others.

A target protein may also be selected from targets for human therapeutic drugs. These include proteins which may be used to restore function in numerous diseases, e.g. polygenic diseases, including for example, target proteins selected from B7.1 and B7, TNFR1, TNFR2, NADPH oxidase, Bcll/Bax and other partners in the apoptosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAK STAT, RXR and similar, HIV 1 protease, HIV 1 integrase, influenza, neuraminidase, hepatitis B reverse transcriptase, sodium channel, multi drug resistance (MDR), protein P-glycoprotein (and MRP), serine/threonine kinases, tyrosine kinases, CD23, CD124, tyrosine kinase p56 Ick, CD4, CD5, IL-2 receptor, IL-1 receptor, TNF-alphaR, ICAM1, Cat+ channels, VCAM, VLA-4 integrin, selectins, CD40/CD40L, neurokinins and receptors, inosine monophosphate dehydrogenase, p38 MAP Kinase, Ras/Raf/MEK/ERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly(ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, adenosine receptors, adenosine kinase and AMP deaminase, purinergic receptors (P2Y1, P2Y2, P2Y4, P2Y6, P2X1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, SMARCA2; protoporphyrinogen oxidase, and enolpyruvylshikimate-phosphate synthase.

Target proteins may also be haloalkane dehalogenase enzymes. By way of example, bifunctional molecules according to the disclosure which contain chloroalkane peptide binding moieties (C1-C12 often about C2-C10 alkyl halo groups) may be used to inhibit and/or degrade haloalkane dehalogenase enzymes which are used in fusion proteins or related diagnostic proteins as described in PCT/US2012/063401 filed Dec. 6, 2011 and published as WO 2012/078559 on Jun. 14, 2012, the contents of which is incorporated by reference herein.

### Target Protein Binding Ligand (TBL)

As used herein, a “target protein binding ligand” refers to a ligand or moiety, which binds to a target protein, e.g. a selected target protein. By way of example, a target protein binding ligand may be any moiety, which selectively and/or specifically binds a target protein. A bifunctional molecule according to this disclosure may comprise a target protein binding ligand, which binds to the target protein with sufficient binding affinity such that the target protein is more susceptible to degradation or proteolysis than if unbound by the bifunctional molecule.

The target protein binding ligand may bind to a target protein with a binding affinity of less than or equal to about 10 μM, less than or equal to about 1 μM, less than or equal to about 0.5 μM, or less than or equal to about 0.1 μM.

In some examples, the ligand may bind to the target protein with a binding affinity of about 0.01 nM to about 10 μM, such as about 0.01 nM to about 8 μM, about 0.01 nM to about 5 μM, about 0.01 nM to about 3 μM.

For the avoidance of doubt, binding affinity is a measure of the propensity of an object comprising two components bound together to separate (dissociate) into the two components. As used herein, the binding affinity is the measure of the propensity of the complex formed when the target protein binding ligand binds to the target protein to dissociate into separate components, i.e. the propensity of the target protein binding ligand to dissociate from the target protein.

The binding between the target protein and the target protein binding ligand may comprise one or more binding interactions, such as one or more of the group consisting of hydrogen bonding, dipole-dipole bonding, ion-dipole bonding, ion-induced dipole bonding, ionic bonding and covalent bonding. For example, the binding between the target protein and the target protein binding ligand may comprise a salt bridge (a combination of hydrogen and ionic bonding).

As stated above, in some examples, the target protein binding ligand moiety may not be a target protein binding ligand selected from: (i) an estrogen receptor binding ligand; and (ii) an androgen receptor binding ligand. In particular, in some examples, the target protein ligand comprised within the bifunctional molecules of the present disclosure is: (i) not a ligand that specifically binds to an estrogen receptor; and (ii) not a ligand that specifically binds to an androgen receptor.

In some examples, the bifunctional molecules disclosed herein do not comprise a target protein binding ligand that binds to an estrogen receptor with a binding affinity of less than or equal to about 10 μM, or less than or equal to about 1 μM.

In other words, in some examples, the bifunctional molecules disclosed herein may not comprise a target protein binding ligand that binds to an estrogen receptor with sufficient binding affinity such that the estrogen receptor is selectively degraded. In particular, if the bifunctional molecules as described herein were to be contacted with an estrogen receptor, the observed DC₅₀ values (for degradation of the estrogen receptor) would be greater than about 10000 nM, or greater than about 1000 nM.

In some examples, the bifunctional molecules disclosed herein may not comprise a target protein binding ligand that binds to an androgen receptor with a binding affinity of less than or equal to about 10 μM, or less than or equal to about 1 μM.

Additionally, in some examples, the bifunctional molecules disclosed herein may not comprise a target protein binding ligand that binds to an androgen receptor with sufficient binding affinity such that the androgen receptor is selectively degraded. In particular, if the bifunctional molecules as described herein were to be contacted with an androgen receptor, the observed DC₅₀ values (for degradation of the androgen receptor) would be greater than about 10000 nM, or greater than about 1000 nM.

A target protein binding ligand may comprise or be derived from a small molecule (or analogue or fragment thereof) already known to act as a modulator, promoter and/or inhibitor of protein function (e.g. any small molecule known to bind to the target protein). By way of example, the target protein binding ligand may comprise or be derived from a small molecule that is known to inhibit activity of a given target protein.

Non-limiting examples of small molecules that can be comprised in the target protein binding ligand moiety of the bifunctional molecules described herein include: (i) binders to kinases (including serine/threonine kinases e.g. RAF, receptor tyrosine kinases and other classes), (ii) compounds binding to bromodomain-containing proteins (including BET family and others), (iii) epigenetic modulator compounds (including binders to histone or DNA methyl transferases, acetyl transferases, deacetylases & demethylases and others e.g. histone deacetylase (HDAC), lysine acetyl transferases such as P300 (EP300; adenoviral E1A binding protein of 300 kDa) and CBP (CREBBP; cyclic-AMP response element binding protein)), (iv) binders to transcription factors including STAT3, myc and others, (v) binders to GTPases (including KRAS, NRAS, HRAS and others), (vi) binders of phosphatases, (vii) binders of ubiquitin E3 ligases (e.g. MDM2) and deubiquitinase enzymes; (viii) binders of nuclear hormone receptors (including, for example, thyroid receptor, androgen receptor (AR) and estrogen receptor (ER), although in some examples, as stated above, the target binding ligands do not comprise binders to androgen receptor and estrogen receptor); (ix) binders to aggregation-prone proteins (including Beta-amyloid, tau, Htt, alpha-synuclein and polyQ-expanded proteins); (x) binders to apoptotic & anti-apoptotic factors (including Bcl2, Bcl-xl and Mcl-1), and (xi) binders to polymerases (including PARP & POLQ) among numerous others.

Other non-limiting examples of small molecules that can be comprised in the target protein binding ligand moiety of the bifunctional molecules described herein include: (i) Hsp90 inhibitors, (ii) human lysine methyltransferase inhibitors, (iii) angiogenesis inhibitors, (iv) compounds targeting the aryl hydrocarbon receptor (AHR), (v) compounds targeting FKBP, (vi) compounds targeting HIV protease, (vii) compounds targeting HIV integrase, (viii) compounds targeting HCV protease, (ix) compounds targeting acyl-protein thioesterase-1 and -2 (APT1 and APT2) among numerous others.

### BET Inhibitors

In some instances, the target protein binding ligand is derived from a BET inhibitor (e.g. the BET inhibitor IBET276). In such examples, the target protein binding ligand may comprise the following structure:

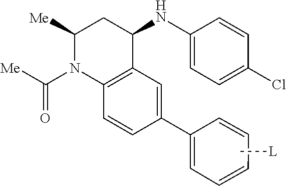

wherein L shows the position of attachment of the linker and the dotted line on the structure above indicates that the linker may be joined to the target protein binding ligand via any position on the aromatic ring (e.g. in some examples, L may be present at the 4-position on this aromatic ring). However, the present disclosure also encompasses joining or coupling to the linker at any chemically suitable position on this target protein binding ligand.

### BRD9

Alternatively, the target protein binding ligand may be derived from a BRD9 inhibitor, for example the target protein binding ligand may comprise the following structure:

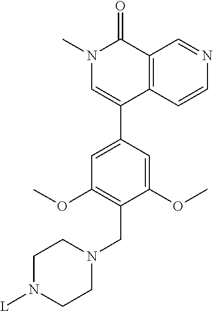

wherein L shows the position of attachment of the linker. The present disclosure also encompasses joining or coupling to the linker at any other chemically suitable position on this target protein binding ligand.

### Kinase Inhibitors

In other examples, the target protein binding ligand is derived from a kinase inhibitor. In such examples, the target protein binding ligand may comprise the following structure:

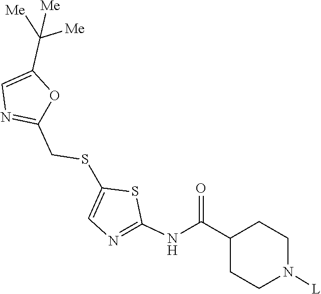

wherein L shows the position of attachment of the linker. However, again, the present disclosure also encompasses joining or coupling to the linker at any chemically suitable position on this target protein binding ligand.

The target protein binding ligand may be derived from a kinase inhibitor, such as a CDK9 inhibitor, and may comprise the following structure:

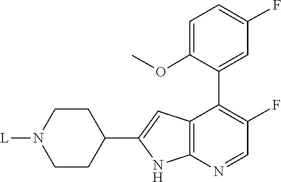

where L shows the position of attachment of the linker. The present disclosure also encompasses joining or coupling to the linker at any other chemically suitable position on this target protein binding ligand.

### EGFR

Alternatively, the target protein binding ligand may be derived from a kinase inhibitor such as a mutant EGFR inhibitor, and may have the following structure:

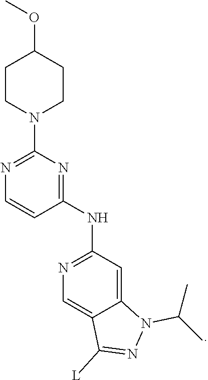

where L shows the position of attachment of the linker. Again, the present disclosure also encompasses joining or coupling to the linker at any other chemically suitable position on this target protein binding ligand.

In some examples, the target protein binding ligand may be represented by formula (EGFR1):

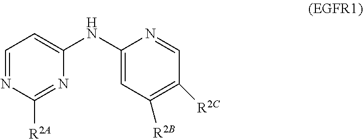

wherein R^{2A} is selected from C₁-C₆alkyl, C₃-C₇cycloalkyl, C₃-C₇heterocycloalkyl, heteroaryl, —O(C₁-C₆alkyl), and —NR^{a1}R^{b1}, wherein said C₁-C₆alkyl, C₃-C₇cycloalkyl, C₃-C₇heterocycloalkyl and heteroaryl are optionally further substituted with one to five R^{f1} groups;
wherein each R^{a1} is independently H or C₁-C₆alkyl;
wherein each R^{b1} is independently H, C₁-C₆alkyl, C₁-C₆alkoxy, amino, —(CH₂)ₘ₁C(O)NH₂, —(CH₂)ₘ₁C₃-C₇cycloalkyl, —(CH₂)ₘ₁C₃-C₇heterocycloalkyl or —(CH₂)ₘ₁heteroaryl;
wherein each R^{f1} is independently selected from the group consisting of C₁-C₆alkyl, C₁-C₆alkoxy, amino, hydroxy, C₁-C₆alkylamino, amide, urea, oxo, halo, pyrazolyl, imidazolyl, triazolyl, CN, —NHC(O)(C₁-C₃alkyl), acyl, sulfonyl, sulfoxide, sulfonamide, sulfoximinyl, —(CH₂)ₘ₁C₃-C₇heterocycloalkyl, and C(O)OR^{a1};
R^{2B} is hydrogen and R^{2C} is —NHC(O)R^{2D} where R^{2D} is selected from aryl and heteroaryl optionally substituted with one to two substituents independently selected from C₁-C₆alkyl, halo, C₁-C₆alkoxy and CN; or
R^{2B} and R^{2C} taken together form a five- to ten-membered aryl or heteroaryl ring optionally substituted with one to five substituents each independently selected from C₁-C₆alkyl, halo, C₁-C₆haloalkyl, C₁-C₆alkoxy, CN, amino, C₁-C₆alkylamino, —O(C₁-C₆haloalkyl), oxo, C₃-C₇cycloalkyl, C₃-C₇heterocycloalkyl optionally substituted with C₁-C₃alkyl, C₁-C₆hydroxyalkyl, aryl and heteroaryl; and
each m1 is independently 0, 1, 2 or 3.

The target protein binding ligand (TBL) represented in formula (EGFR1) may be appended to the linker L of the bifunctional molecule by way of a covalent bond between an atom on the target protein binding ligand (TBL) and an atom on the linker (L). The linker may be attached at any suitable position e.g. provided it has the correct valency and/or is chemically suitable. For example, the linker of the bifunctional molecule may be covalently bonded to the R^{2B} or R^{2C} moiety of formula (EGFR1) at any position (provided that it has the correct valency and/or is chemically suitable).

In some examples of formula (EGFR1), R^{2A} is C₃-C₇heterocycloalkyl. In particular, R^{2A} may be a C₃-C₇heterocycloalkyl comprising at least one N ring atom that is optionally substituted with one to three R^{f1} groups (as defined above). In some examples, R^{2A} may comprise the following structure:

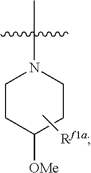

wherein R^{f1} is halo; and wherein the wavy line bisects the bond that forms the attachment to the pyrimidinyl core of formula (EGFR1).

In some examples, the target protein binding ligand may be represented by formula (EGFR2):

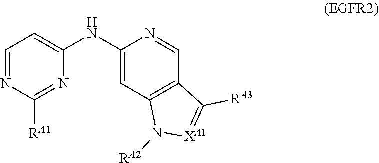

wherein R^{A1} is selected from C₃-C₇heterocycloalkyl, heteroaryl, —O(C₁-C₆alkyl), or —NR^{a1}R^{b1} wherein said C₃-C₇heterocycloalkyl and heteroaryl are optionally further substituted with one to five R^{f1} groups;
R^{A2} is selected from H, C₁-C₆alkyl, C₁-C₆haloalkyl, C₃-C₇cycloalkyl and C₃-C₇heterocycloalkyl;
R^{A3} is absent or is selected from C₁-C₆alkyl, C₃-C₇heterocycloalkyl, C₃-C₇cycloalkyl, —O(C₁-C₆alkyl), CN, —NR^{a1}R^{a1}, —NHC(O)(C₁-C₃alkyl), —C(O)NR^{a1}R^{b1} and heteroaryl;
wherein each R^{f1} is independently selected from the group consisting of C₁-C₆alkyl, C₁-C₆alkoxy, amino, hydroxy, C₁-C₆alkylamino, amide, urea, oxo, halo, pyrazolyl, imidazolyl, triazolyl, CN, —NHC(O)(C₁-C₃alkyl), acyl, sulfonyl, sulfoxide, sulfonamide, sulfoximinyl, —(CH₂)ₘ₁C₃-C₇heterocycloalkyl, and C(O)OR^{a1};
wherein each R^{a1} is independently H or C₁-C₆alkyl;
wherein each R^{b1} is independently H, C₁-C₆alkyl, C₁-C₆alkoxy, amino, —(CH₂)ₘ₁C(O)NH₂, —(CH₂)ₘ₁C₃-C₇cycloalkyl, —(CH₂)ₘ₁C₃-C₇heterocycloalkyl or —(CH₂)ₘ₁heteroaryl;
or R^{a1} and R^{b1} together may form a C₃-C₇cycloalkyl, C₃-C₇heterocycloalkyl or heteroaryl ring, wherein said C₃-C₇cycloalkyl, C₃-C₇heterocycloalkyl and heteroaryl may each be further substituted with one to three groups selected from the group consisting of halo, hydroxy, C₁-C₃alkyl, amino, oxo, amide, sulfonyl, sulfoxide, sulfoximinyl, sulfonamide, C₁-C₃alkoxy, CN and acyl;
X^{A1} is selected from N and CH; and
each m1 is independently 0, 1, 2 or 3.

In some examples of formula (EGFR2), X^{A1} is N.

In some examples of formula (EGFR2), R^{A1} is selected from C₃-C₇heterocycloalkyl, heteroaryl and NR^{a1}R^{b1}.

In some examples of formula (EGFR2), R^{A1} is selected from C₃-C₇heterocycloalkyl and heteroaryl, the C₃-C₇heterocycloalkyl and heteroaryl being optionally further substituted with one to five R^{f1} groups, wherein each R^{f1} is independently selected from sulfonyl, alkoxy and halo; R^{A3} is absent or is —C(O)NR^{a1}R^{b1}; and R^{A2} is C₁-C₆alkyl.

In some examples of formula (EGFR2), R^{A1} is a C₅-C₇heterocycloalkyl containing at least one N heteroatom (e.g. piperidinyl) or is a heteroaryl containing at least one N heteroatom in the ring (e.g. pyrazolyl).

In some examples of formula (EGFR2), R^{f1} is selected from —SO₂(C₃-C₇cycloalkyl) (e.g. —SO₂(cyclopropyl)), halo (e.g. F), hydroxy, C₁-C₆alkyl and C₁-C₆alkoxy (e.g. methoxy).

In some examples of formula (EGFR2), R^{A2} is C₁-C₆alkyl or C₁-C₆haloalkyl, such as C₁-C₄alkyl or C₁-C₄haloalkyl. Representative examples of R^{A2} include, but are not limited to, isopropyl, sec-butyl and 1,1,1-trifluoropropan-2-yl.

In some examples of formula (EGFR2), R^{A3} is selected from C₁-C₃alkyl, C₃-C₇heterocycloalkyl, heteroaryl, —NR^{a1}R^{a1} and —C(O)NR^{a1}R^{b1}.

In some examples of formula (EGFR2), R^{A1} is selected from C₃-C₇heterocycloalkyl (e.g. piperidinyl) and heteroaryl (e.g. pyrazolyl), wherein said C₃-C₇heterocycloalkyl or heteroaryl is optionally further substituted with one to three R^{f1} groups selected from C₁-C₆alkyl, C₁-C₆alkoxy, hydroxy, halo, sulfonyl, and sulfonamide.

In some examples, the target protein binding ligand may be represented by formula (EGFR2a):

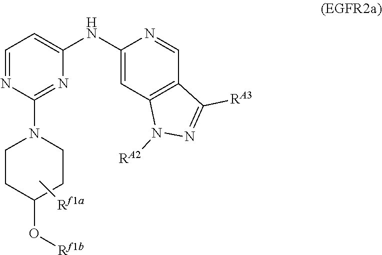

wherein R^{A2} and R^{A3} are as defined in formula (2); and
R^{f1b} is selected from H and C₁-C₆alkyl (e.g. C₁-C₃alkyl); and
R^{f1a} is absent or is halo.

The target protein binding ligand (TBL) represented in formulae (EGFR2) and (EGFR2a) may be appended to the linker L of the bifunctional molecule by way of a covalent bond between an atom on the target protein binding ligand (TBL) and an atom on the linker (L). The linker may be attached at any suitable position e.g. provided it has the correct valency and/or is chemically suitable. For example, the linker of the bifunctional molecule may be covalently bonded to the R^{A3} moiety of formula (EGFR2) or (EGFR2a) at any position (provided that it has the correct valency and/or is chemically suitable). In those examples where R^{A3} is absent, the linker of the bifunctional molecule may be covalently bonded to the heteroaryl ring as shown on formulae (EGFR2) and (EGFR2a).

In some examples, the target protein binding ligand may be represented by formula (EGFR3):

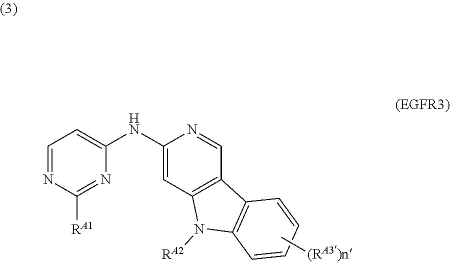

wherein R^{A1} is selected from C₃-C₇heterocycloalkyl, heteroaryl, —O(C₁-C₆alkyl), or —NR^{a1}R^{b1},
wherein said C₃-C₇heterocycloalkyl and heteroaryl are optionally further substituted with one to five R^{f1} groups;
R^{A2} is selected from H, C₁-C₆alkyl, C₁-C₆haloalkyl, C₃-C₇cycloalkyl and C₃-C₇heterocycloalkyl;
R^{A3}′ is absent or is selected from C₁-C₆alkyl, halo, C₁-C₆haloalkyl, C₁-C₆alkoxy, CN, amino, C₁-C₆alkylamino, di(C₁-C₆alkyl)amino, —O(C₁-C₆haloalkyl), oxo, C₃-C₇cycloalkyl, C₃-C₇heterocycloalkyl optionally substituted with C₁-C₃alkyl, C₁-C₇hydroxyalkyl, aryl and heteroaryl;
wherein each R^{f1} is independently selected from the group consisting of C₁-C₆alkyl, C₁-C₆alkoxy, amino, hydroxy, C₁-C₆alkylamino, amide, urea, oxo, halo, pyrazolyl, imidazolyl, triazolyl, CN, —NHC(O)(C₁-C₃alkyl), acyl, sulfonyl, sulfoxide, sulfonamide, sulfoximinyl, —(CH₂)ₘ₁C₃-C₇heterocycloalkyl, —O(C₁-C₆alkyl) and C(O)OR^{a1};
wherein each R^{a1} is independently H or C₁-C₆alkyl;
wherein each R^{b1} is independently H, C₁-C₆alkyl, C₁-C₆alkoxy, amino, —(CH₂)ₘ₁C(O)NH₂, —(CH₂)ₘ₁C₃-C₇cycloalkyl, —(CH₂)ₘ₁C₃-C₇heterocycloalkyl or —(CH₂)ₘ₁heteroaryl;
or R^{a1} and R^{b1} together may form a C₃-C₇cycloalkyl, C₃-C₇heterocycloalkyl or heteroaryl ring, wherein said C₃-C₇cycloalkyl, C₃-C₇heterocycloalkyl and heteroaryl may each be further substituted with one to three groups selected from the group consisting of halo, hydroxy, C₁-C₃alkyl, amino, oxo, amide, sulfonyl, sulfoxide, sulfoximinyl, sulfonamide, C₁-C₃alkoxy, CN and acyl;
each m1 is independently 0, 1, 2 or 3; and
n′ is 0, 1 or 2.

In those examples where n′ is 2, each R^{A3}′ is independently selected from C₁-C₆alkyl, halo, C₁-C₆haloalkyl, C₁-C₆alkoxy, CN, amino, C₁-C₆alkylamino, —O(C₁-C₆haloalkyl), oxo, C₃-C₇cycloalkyl, C₃-C₇heterocycloalkyl optionally substituted with C₁-C₃alkyl, C₁-C₆hydroxyalkyl, aryl and heteroaryl.

In some examples of formula (3), R^{A1} is selected from C₃-C₇heterocycloalkyl and heteroaryl, wherein said C₃-C₇heterocycloalkyl and heteroaryl are optionally further substituted with one to five R^{f1} groups as defined above. By way of further example, R^{A1} may be selected from C₅-C₇heterocycloalkyl containing at least one N ring atom (e.g. piperidinyl) and a five—to six-membered heteroaryl containing one, two or three N ring atoms (e.g. pyrazolyl), both of which may optionally be further substituted with one to five R^{f1} groups as defined above.

In some examples, the target protein binding ligand may be represented by formula (EGFR3a):

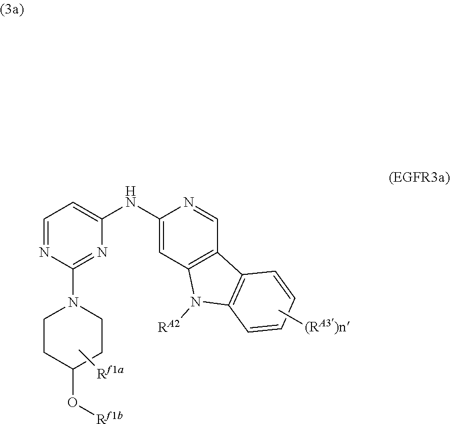

wherein R^{A2}, R^{A3}′ and n′ are as defined in formula (EGFR3); and
R^{f1b} is selected from H and C₁-C₆alkyl (e.g. C₁-C₃alkyl); and
R^{f1a} is absent or is halo.

In some examples of formula (EGFR3) or (EGFR3a), R^{A2} may be C₁-C₆alkyl (such as isopropyl).

In some examples of formula (EGFR3) or (EGFR3a), R^{A3}′ may be selected from C₁-C₆alkyl, halo, C₁-C₆haloalkyl, C₃-C₇heterocycloalkyl optionally substituted with C₁-C₃alkyl and —O(C₁-C₆haloalkyl).

In some examples of formula (3) or (3a), n′ is 2 and a first R^{A3}′ is —O(C₁-C₆haloalkyl) and a second R^{A3}′ is C₃-C₇heterocycloalkyl optionally substituted with C₁-C₃alkyl.

In some examples of formula (3) or (3a), n′ is 1 and R^{A3}′ is —O(C₁-C₆haloalkyl).

The target protein binding ligand (TBL) represented in formulae (EGFR3) and (EGFR3a) may be appended to the linker L of the bifunctional molecule by way of a covalent bond between an atom on the target protein binding ligand (TBL) and an atom on the linker (L). The linker may be attached at any suitable position e.g. provided it has the correct valency and/or is chemically suitable. For example, the linker of the bifunctional molecule may be covalently bonded to the R^{A3}′ moiety of formula (EGFR3) or (EGFR3a) at any position (provided that it has the correct valency and/or is chemically suitable). In those examples where R^{A3}′ is absent, the linker of the bifunctional molecule may be covalently bonded to the heteroaryl ring as shown on formulae (EGFR3) and (EGFR3a).

Representative examples of target protein binding ligands that may be incorporated into the bifunctional molecules of the present disclosure are shown below:

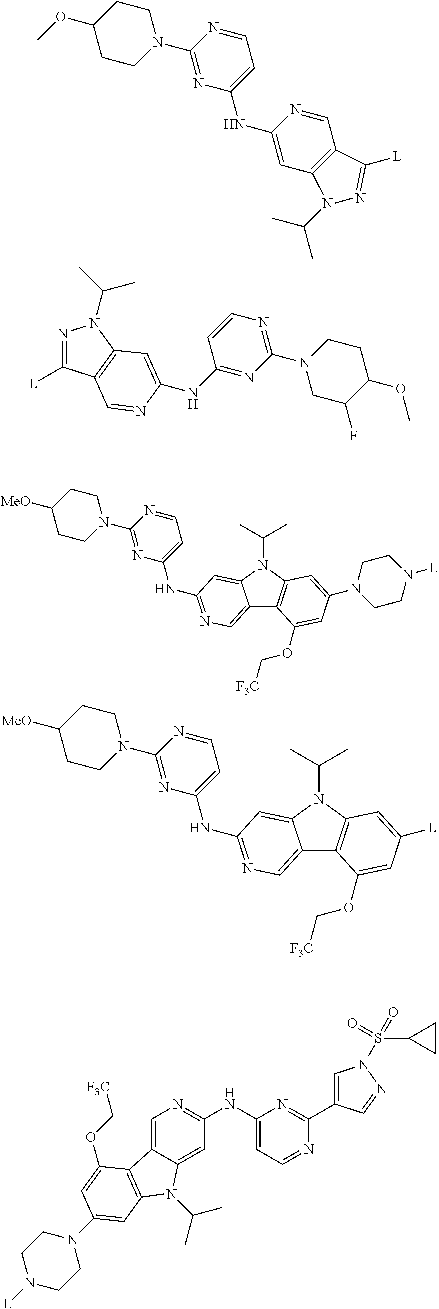

Where L on the structures above represents the point of attachment of the linker. Again, the present disclosure also encompasses joining or coupling to the linker at any other chemically suitable position on this target protein binding ligand.

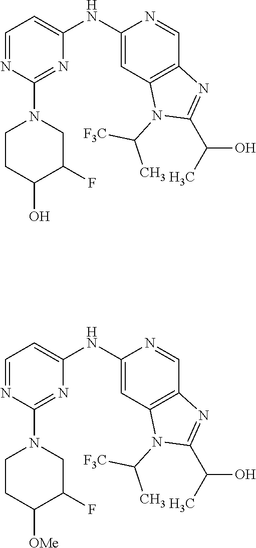 [SMILES: CC(O)c1nc2cnc(Nc3ccnc(N4CCC(O)C(F)C4)n3)cc2n1C(C)C(F)(F)F ; COC1CCN(c2nccc(Nc3cc4c(cn3)nc(C(C)O)n4C(C)C(F)(F)F)n2)CC1F]

As above, the linker may be attached to the target protein binding ligand at any suitable position e.g. provided it has the correct valency and/or is chemically suitable.

As shown on the structures above, the TBL moiety may comprise one or more chiral centres. As stated previously, the present disclosure encompasses each individual enantiomer of these TBL moieties as well as mixtures of enantiomers including racemic mixtures of such enantiomers. By way of further example, where the TBL moiety comprises two or more chiral centres, the present disclosure encompasses each individual diastereomer of the TBL moiety, as well as mixtures of the various diastereomers.

By way of particular example, the target protein binding ligand that may be incorporated into the bifunctional molecules of the present disclosure may be represented as:

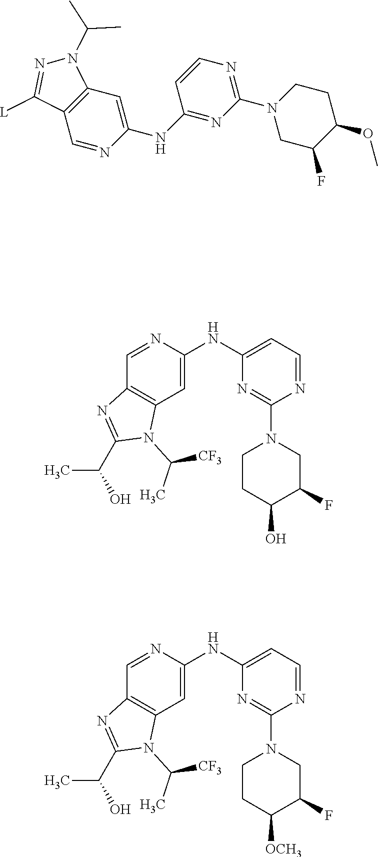

### KRAS

In some instances, the target protein binding ligand may be derived from a GTPase inhibitor, such as a KRAS G12C inhibitor. For example, the target protein binding ligand may have the following structure:

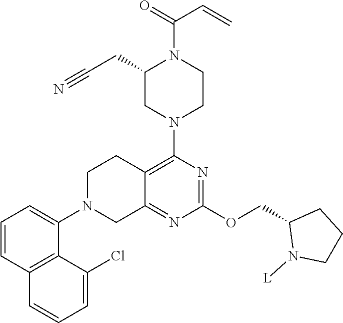

where L shows the position of attachment of the linker. Again, the present disclosure also encompasses joining or coupling to the linker at any other chemically suitable position on this target protein binding ligand.

In some instances, the target protein binding ligand may be derived from a GTPase inhibitor, such as a KRAS G12D inhibitor. For example, the target protein binding ligand may have the following structure:

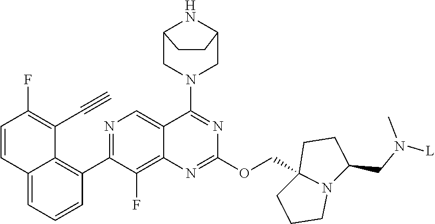

where L shows the position of attachment of the linker. Again, the present disclosure also encompasses joining or coupling to the linker at any other chemically suitable position on this target protein binding ligand.

Accordingly, in some examples, the target protein binding ligand may be represented by formula (KRAS1):

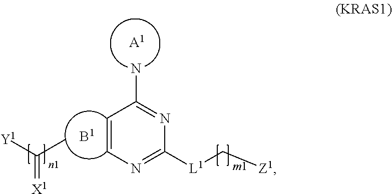

wherein:
ring A¹ represents an optionally substituted saturated or unsaturated 5- to 10-membered N-containing ring which contains at least one further heteroatom selected from the group consisting of N, S and O and is optionally bridged;
ring B¹ represents an optionally substituted moiety selected from the group consisting of a 5- to 6-membered unsaturated ring having at least one heteroatom selected from the group consisting of N, S, and O, a 6- to 10-membered (e.g. 6-membered) aromatic hydrocarbon ring, a C₃₋₆cycloalkene ring and an 8- to 10-membered spiro ring, wherein the ring B¹ is fused with the pyrimidine ring to form a substituted or unsubstituted bicyclic ring;
n1 is 0 or 1;
X¹ is O or S;
Y¹ is an optionally substituted moiety selected from the group consisting of a 6- to 10-membered aromatic hydrocarbyl ring and a 6- to 10-membered unsaturated monocyclic or bicyclic ring which contains at least one heteroatom selected from the group consisting of N, S and O;
L₁ is absent or is any one moiety selected from the group consisting of O, optionally substituted C₂₋₃alkynylene and NR^{c};
Z¹ is:
(i) any one moiety selected from the group consisting of cyanoalkyl, alkylcarbonylaminoalkyl, alkylaminocarbonyl, alkylaminoalkyl, optionally substituted C₃₋₆cycloalkyl, a 5- to 6-membered saturated ring which contains at least one heteroatom selected from the group consisting of N, S and O, and an 8- to 10-membered partially unsaturated ring which contains at least one heteroatom selected from the group consisting of N, S and O; or
(ii) any one moiety selected from the group consisting of hydrogen, —N(R^{C})₂, heterocyclyl, C₁₋₆alkyl, -D-heterocyclyl, -D-aryl, -D-heteroaryl, -D-cycloalkyl, -D-N(R^{C})₂, -D-NHC(═NH)NH₂, -D-C(O)N(R^{C})₂, -D-C₁₋₆ haloalkyl, -D-ORG, -D-(CH₂OR^{C}) (CH₂)ₙOR^{C}, -D-NR^{C}C(O)-aryl, -D-COOH, and -DC(O)OC₁₋₆alkyl, wherein the heterocyclyl and the aryl portion of -D-NRCC(O)-aryl and the heterocyclyl portion of -D-heterocyclyl and the cycloalkyl portion of the -D-cycloalkyl may be optionally substituted with one or more R^{D}, and wherein the aryl or heteroaryl of the -D-aryl and the -D-heteroaryl may be optionally substituted with one or more R^{E}; wherein
each D is independently a C₁₋₄alkylene optionally substituted with hydroxy, C₁₋₄hydroxyalkyl or heteroaryl;
each R^{C} is independently hydrogen or C₁₋₃alkyl;
each R^{D} is independently halo, hydroxy, C₁₋₃hydroxyalkyl, C₁₋₃alkyl, C₁₋₃haloalkyl, C₁₋₃alkoxy, cyano, -Q-phenyl, -Q-phenylSO₂F, —NHC(O)phenyl, —NHC(O)phenylSO₂F, C₁₋₃alkyl substituted pyrazolyl, araC₁₋₃alkyl-, tert-butyldimethylsilyloxyCH₂—, —N(R^{C})₂, (C₁₋₃alkoxy)C₁₋₃alkyl-, (C₁₋₃alkyl)C(O), oxo, (C₁₋₃haloalkyl)C(O)—, —SO₂F, (C₁₋₃alkoxy)C₁₋₃alkoxy, —CH₂OC(O)N(R^{C})₂, —CH₂NHC(O)OC₁₋₆alkyl, —CH₂NHC(O)N(R^{C})₂, —CH₂NHC(O)C₁₋₆alkyl, —CH₂(pyrazolyl), —CH₂NHSO₂C₁₋₆alkyl, —CH₂OC(O)heterocyclyl, —OC(O)N(R^{C})₂, —OC(O)NH(C₁₋₃alkyl)O(C₁₋₃alkyl), —OC(O)NH(C₁₋₃alkyl)O(C₁₋₃alkyl)phenyl(C₁₋₃alkyl)N(CH₃)₂, —OC(O)NH(C₁₋₃alkyl)O(C₁₋₃alkyl)phenyl or —OC(O)heterocyclyl, —CH₂heterocyclyl, wherein the phenyl of —NHC(O)phenyl or —OC(O)NH(C₁₋₃alkyl)O(C₁₋₃alkyl)phenyl is optionally substituted with —C(O)H or OH and wherein the heterocyclyl of —CH₂heterocyclyl is optionally substituted with OXO;
Q is a bond or O;
each R^{E} is independently halogen, hydroxy, HC(O)—, C₁₋₄alkyl, C₁₋₄alkoxy, C₁₋₄haloalkyl, C₁₋₄hydroxyalkyl, or —N(R^{C})₂; and
m1 is 0 to 3.

The linker may be attached the structure of formula (1) at any suitable position, for example where valency allows substitution or covalent addition of a linker moiety. Suitably, the linker is attached to the TBL via suitably substitution on Z¹.

In some examples of formula (KRAS1), when L₁ is C₂₋₃alkynylene, Z¹ is alkylaminocarbonyl or alkylaminoalkyl and m1 is 0 or 1.

In some examples of formula (KRAS1), when Z¹ is as defined in (ii), m1 is 0.

The optional substituent of Y¹ of formula (KRAS1) may be one or more substituents independently selected from the group consisting of halo, cyano, hydroxy, C₁₋₄alkyl, —S—C₁₋₃alkyl, C₂₋₄alkenyl, C₂₋₄alkynyl, C₂₋₄hydroxyalkynyl, C₁₋₃cyanoalkyl, triazolyl, C₁₋₃haloalkyl, —O—C₁₋₃haloalkyl, —S—C₁₋₃haloalkyl, C₁₋₃alkoxy, hydroxyC₁₋₃alkyl, —CH₂C(O)N(R^{C})₂, —C₃₋₄alkyny(NR^{C})₂, —N(R^{C})₂, deuteroC₂₋₄alkynyl, (C₁₋₃alkoxy)haloC₁₋₃alkyl-, and C₃₋₆cycloalkyl, wherein said C₃₋₆cycloalkyl is optionally substituted with halo or C₁₋₃alkyl.

The target protein binding ligand represented in formula (KRAS1) may be appended to the linker L of the bifunctional molecule by way of a covalent bond between an atom on the target protein binding ligand (TBL) and an atom on the linker (L). The linker may be attached at any suitable position e.g. provided it has the correct valency and/or is chemically suitable. For example, the linker of the bifunctional molecule may be covalently bonded to the Z¹ moiety of formula (KRAS1) at any position (provided that it has the correct valency and/or is chemically suitable).

In some examples, the target protein binding ligand may be represented by formula (KRAS2):

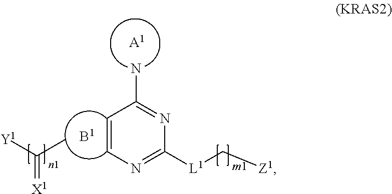

wherein:
ring A¹ represents an optionally substituted saturated or unsaturated 8- to 10-membered N-containing bridged ring which contains at least one further heteroatom selected from the group consisting of N, S and O;
ring B¹ represents an optionally substituted moiety selected from the group consisting of a 5-to 6-membered unsaturated ring having at least one heteroatom selected from the group consisting of N, S, and O, a 6-membered aromatic hydrocarbon ring, a C₃₋₆cycloalkene ring and an 8- to 10-membered spiro ring, wherein the ring B¹ is fused with the pyrimidine ring to form a substituted or unsubstituted bicyclic ring;
n1 is 0 or 1;
X¹ is O or S;
Y¹ is an optionally substituted moiety selected from the group consisting of a 6- to 10-membered aromatic hydrocarbyl ring and a 6- to 10-membered unsaturated monocyclic or bicyclic ring which contains at least one heteroatom selected from the group consisting of N, S and O;
L₁ is O or an optionally substituted C₂₋₃alkynylene;
Z¹ is any one moiety selected from the group consisting of cyanoalkyl, alkylcarbonylaminoalkyl, alkylaminocarbonyl, alkylaminoalkyl, optionally substituted C₃₋₆cycloalkyl, a 5- to 6-membered saturated ring which contains at least one heteroatom selected from the group consisting of N, S and O, and an 8- to 10-membered partially unsaturated ring which contains at least one heteroatom selected from the group consisting of N, S and O; and
m1 is 0 to 3 (e.g. m1 is 0 or 1).

Suitably, L or L-Z of the degrader is joined to the Z¹ moiety.

In some examples of formula (KRAS2), when L₁ is optionally substituted C₂₋₃alkynylene, Z¹ is alkylaminocarbonyl or alkylaminoalkyl.

In some specific examples, the target protein binding ligand may be represented by formula (KRAS3):

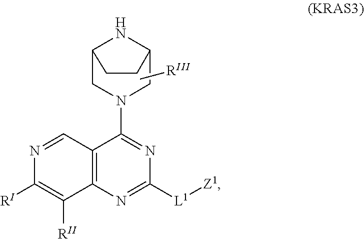

wherein:
L₁ is absent or is any one moiety selected from the group consisting of O and NR^{C};
Z¹ is:
(ii) any one moiety selected from the group consisting of hydrogen, —N(R^{C})₂, heterocyclyl, C₁₋₆alkyl, -D-heterocyclyl, -D-aryl, -D-heteroaryl, -D-cycloalkyl, -D-N(R^{C})₂, -D-NHC(═NH)NH₂, -D-C(O)N(R^{C})₂, -D-C₁₋₆ haloalkyl, -D-ORG, -D-(CH₂OR^{C})(CH₂)ₙORG, -D-NR^{C}C(O)-aryl, -D-COOH, and -DC(O)OC₁₋₆alkyl, wherein the heterocyclyl and the aryl portion of -D-NR^{C}C(O)-aryl and the heterocyclyl portion of -D-heterocyclyl and the cycloalkyl portion of the -D-cycloalkyl may be optionally substituted with one or more R^{D}, and wherein the aryl or heteroaryl of the -D-aryl and the -D-heteroaryl may be optionally substituted with one or more R^{E}; wherein
each D is independently a C₁₋₄alkylene optionally substituted with hydroxy, C₁₋₄hydroxyalkyl or heteroaryl;
each R^{C} is independently hydrogen or C₁₋₃alkyl;
each R^{D} is independently halo, hydroxy, C₁₋₃hydroxyalkyl, C₁₋₃alkyl, C₁₋₃haloalkyl, C₁₋₃alkoxy, cyano, -Q-phenyl, -Q-phenylSO₂F, —NHC(O)phenyl, —NHC(O)phenylSO₂F, C₁₋₃alkyl substituted pyrazolyl, araC₁₋₃alkyl-, tert-butyldimethylsilyloxyCH₂—, —N(R^{C})₂, (C₁₋₃alkoxy)C₁₋₃alkyl-, (C₁₋₃alkyl)C(O), oxo, (C₁₋₃haloalkyl)C(O)—, —SO₂F, (C₁₋₃alkoxy)C₁₋₃alkoxy, —CH₂OC(O)N(R^{C})₂, —CH₂NHC(O)OC₁₋₆alkyl, —CH₂NHC(O)N(R^{C})₂, —CH₂NHC(O)C₁₋₆alkyl, —CH₂(pyrazolyl), —CH₂NHSO₂C₁₋₆alkyl, —CH₂OC(O)heterocyclyl, —OC(O)N(R^{C})₂, —OC(O)NH(C₁₋₃alkyl)O(C₁₋₃alkyl), —OC(O)NH(C₁₋₃alkyl)O(C₁₋₃alkyl)phenyl(C₁₋₃alkyl)N(CH₃)₂, —OC(O)NH(C₁₋₃alkyl)O(C₁₋₃alkyl)phenyl or —OC(O)heterocyclyl, —CH₂heterocyclyl, wherein the phenyl of —NHC(O)phenyl or —OC(O)NH(C₁₋₃alkyl)O(C₁₋₃alkyl)phenyl is optionally substituted with —C(O)H or OH and wherein the heterocyclyl of —CH₂heterocyclyl is optionally substituted with oxo;
Q is a bond or O;
each R^{E} is independently halo, hydroxy, HC(O)—, C₁₋₄alkyl, C₁₋₄alkoxy, C₁₋₄haloalkyl, C₁₋₄₋hydroxyalkyl, or —N(R^{C})₂;
R^{I} is aryl or heteroaryl, wherein the aryl or the heteroaryl is optionally substituted with one or more substituents independently selected from the group consisting of halo, cyano, hydroxy, C₁₋₄alkyl, —S—C₁₋₃alkyl, C₂₋₄alkenyl, C₂₋₄alkynyl, C₂₋₄hydroxyalkynyl, C₁₋₃cyanoalkyl, triazolyl, C₁₋₃haloalkyl, —O—C₁₋₃haloalkyl, —S—C₁₋₃haloalkyl, C₁₋₃alkoxy, hydroxyC₁₋₃alkyl, —CH₂C(O)N(R^{C})₂, —C₃₋₄alkyny(NR)₂, —N(R^{C})₂, deuteroC₂₋₄alkynyl, (C₁₋₃alkoxy)haloC₁₋₃alkyl-, or C₃₋₆cycloalkyl wherein said C₃₋₆cycloalkyl is optionally substituted with halo or C₁₋₃alkyl;
R^{II} is hydrogen, halo, or C₁₋₃alkyl; and
R^{III} is hydrogen, hydroxy, halo, C₁₋₃alkyl, C₁₋₃cyanoalkyl, C₁₋₃hydroxyalkyl, HC(O)—, —CO₂R^{C}, —CO₂N(R^{C})₂ or a 5-6 membered heteroaryl.

### Ring A¹ of Formula (KRAS1):

Ring A¹ of formula (KRAS1) represents an optionally substituted saturated or unsaturated 5-to 10-membered N-containing ring containing at least one further heteroatom selected from the group consisting of N, S and O and is optionally bridged. Ring A¹ is often an optionally substituted, optionally bridged saturated 6- to 8-membered N-containing ring. Typically, A¹ contains two heteroatoms independently selected from N or O. Often, both heteroatoms are N. In some cases, A¹ is bridged, for example by a methylene, ethylene or propylene (typically a methylene or ethylene) bridge, wherein the bridge is optionally substituted, for example with one or more moieties selected from the group consisting of halo and hydroxy group. In some cases, ring A¹ is an optionally substituted piperazinyl ring, optionally bridged between two of the carbon atoms of the ring.

In some examples, A¹ is an optionally substituted piperazinyl ring, optionally bridged between two of the carbon atoms of the ring wherein the piperazinyl ring is optionally substituted at nitrogen with hydroxy and is optionally substituted at carbon with one or more substituents selected from the group consisting of halo, alkoxycarbonyl, cyano, and hydroxyalkyl.

In some cases, ring A¹ is of formula (KRAS1a):

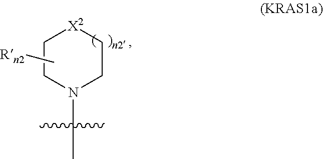

wherein:
X² is NH, N(C₁₋₆alkyl), NOH or O;
n2′ is 1 or 2;
R′ is the optional substituent as defined above; and
n2 is 0 to 8 wherein when n2 is 2 to 4, two of R′ may join to form a bridge between two different carbon atoms of the ring.
R′ is often a substituent independently selected from the group consisting of C₁₋₆alkyl, C₁₋₆alkenyl, halo, alkoxycarbonyl, cyano or hydroxyalkyl (e.g. C₁₋₆₋alkyl). n2 is suitably 1 or 2. When n2 is 2, the two R′ may join to form a bridge between two different carbon atoms of the ring.

In some examples, A¹ is of the following formula:

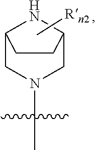

wherein:
R′ is one or more substituent(s) selected from the group consisting of halo, hydroxy, C₁₋₃alkyl, C₁₋₃cyanoalkyl, C₁₋₃hydroxyalkyl, HC(═O)—, —CO₂R^{C}, or —CO₂N(R^{C})₂; and
n2 is 0 to 6;
and wherein R^{C} is defined above for formula (KRAS3).

In some examples, A¹ may be any one moiety selected from the group consisting of:

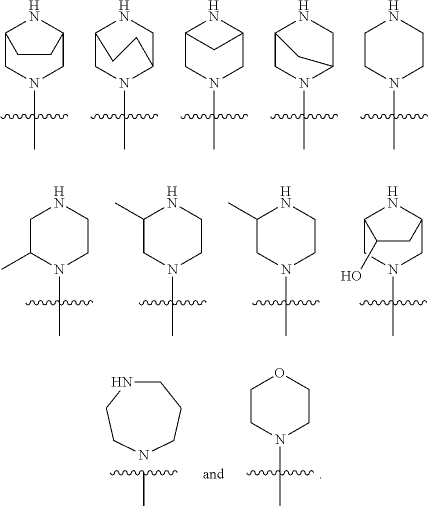

In some cases, A¹ is any one ring selected from the group consisting of:

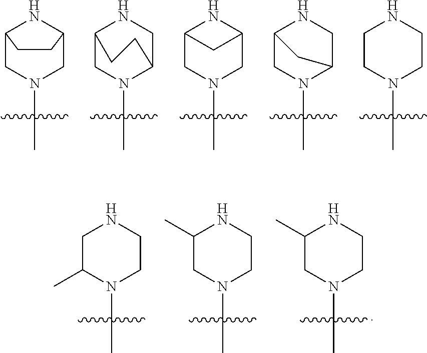

For example, A¹ may be

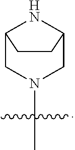 [SMILES: *N1CC2CCC(C1)N2]

### Ring B¹ of Formula (KRAS1)

Ring B¹ of formula (KRAS1) represents an optionally substituted moiety selected from the group consisting of a 5- to 6-membered unsaturated ring having at least one heteroatom selected from the group consisting of N, S, and O, a 6-membered aromatic hydrocarbon ring, a C₃₋₆cycloalkene and an 8- to 10-membered spiro ring, wherein the ring B¹ is fused with the pyrimidine ring to form a substituted or unsubstituted bicyclic ring.

Often, B¹ is an optionally substituted moiety selected from the group consisting of a 5- to 6-membered saturated or unsaturated ring having at least one heteroatom selected from the group consisting of N and O, a phenyl ring, and a C₅₋₆cycloalkene. Often, B¹ is 6-membered. Sometimes, B¹ is aromatic.

In some examples of formula (KRAS1), B¹ is:
(i) a 5- to 6-membered saturated or unsaturated ring which contains at least one heteroatom selected from the group consisting of N, S, and O (such as N or O),
(ii) a 6- to 10-membered aromatic hydrocarbon ring,
(iii) C₃₋₆cycloalkenyl or
(iv) an 8- to 10-membered spiro ring; wherein B¹ is fused with the pyrimidine ring to form a substituted or unsubstituted bicyclic ring; and wherein B¹ in the bicyclic ring may be substituted with one or more substituents selected from the group consisting of halo, C₁₋₆alkyl, alkylcarbonyl and 4- to 6-membered saturated monocyclic heterocyclyl which contains one or more heteroatoms selected from N, S, and O.

In some examples, B¹ is selected from the group consisting of benzene, piperidine, pyrrolidine, cyclohexane, cyclohexene, tetrahydro-2H-pyran, 3,4-dihydro-2H-pyran and spiro[2.5]octane; and is optionally substituted with one or more substituents selected from the group consisting of halo, C₁₋₆alkyl, alkylcarbonyl and oxetanyl.

In some examples, B¹ is selected from the group consisting of benzene, piperidine, pyrrolidine, tetrahydro-2H-pyran and 3,4-dihydro-2H-pyran.

Sometimes, when B¹ is pyrrolidine, n1 is 1 and X¹ is O or S, and when B¹ is not pyrrolidine, n1 is 0.

Typically, B¹ is an optionally substituted 6-membered N-heteroaromatic ring, such as an optionally substituted pyridine ring.

In some cases, B¹ is any one ring selected from the group consisting of:

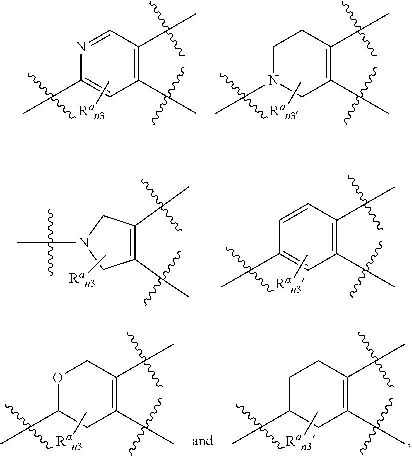

wherein:
n3 is 0 to 2;
n3′ is 0 to 3; and
R^{a} is the optional substituent as defined above; and wherein in these structures, the wavy lines are shown over the bond(s) that forms the link with the parent structure (as shown in formulae (KRAS1), (KRAS2) and (KRAS3).

Typically, the optional substituent of B¹ is one or more moieties selected from the group consisting of halo (such as fluoro or chloro) and C₁₋₄alkyl (such as methyl or ethyl). Often, the optional substituent of B¹ is one or more moieties selected from the group consisting of fluoro, chloro and ethyl. Often, B¹ is unsubstituted or substituted with fluoro.

In some cases, B¹ is any one ring selected from the group consisting of:

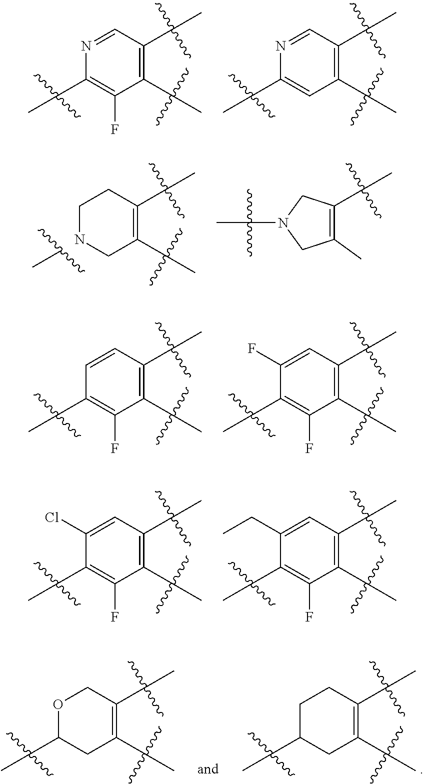

For example, B¹ may be

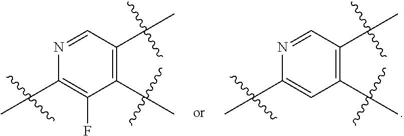 [SMILES: *c1cnc(*)c(F)c1* ; *c1cc(*)c(*)cn1]

Again, in any of the structures shown above, the wavy lines are shown over the bonds that forms the link with the parent structure (as shown in formulae (KRAS1), (KRAS2) and (KRAS3)).

Moiety L₁-(CH₂)ₘ₁—Z¹ of Formula (KRAS1):

L₁ is absent or is any one moiety selected from the group consisting of O, optionally substituted C₂₋₃alkynylene and NR^{c}, wherein R^{c} is as defined above in formula (KRAS1) or (KRAS3).
m1 is 0 to 3.

In some cases, L₁ is O and m1 is 1 or 2. Sometimes, L₁ is O and m is 1.
Z¹ is:
(i) any one moiety selected from the group consisting of cyanoalkyl, alkylcarbonylaminoalkyl, alkylaminocarbonyl, alkylaminoalkyl, optionally substituted C₃₋₆cycloalkyl, a 5- to 6-membered saturated ring which contains at least one heteroatom selected from the group consisting of N, S and O, and an 8- to 10-membered partially unsaturated ring which contains at least one heteroatom selected from the group consisting of N, S and O; or
(ii) any one moiety selected from the group consisting of —N(R^{C})₂, heterocyclyl, C₁₋₆alkyl, -D-heterocyclyl, -D-aryl, -D-heteroaryl, -D-cycloalkyl, -D-N(R^{C})₂, -D-NHC(═NH)NH₂, -D-C(O)N(R^{C})₂, -D-C₁₋₆ haloalkyl, -D-OR^{C}, -D —(CH₂OR^{C})(CH₂)ₙOR^{C}, -D-NR^{C}C(O)-aryl, -D-COOH, and -DC(O)OC₁₋₆alkyl, wherein the heterocyclyl and the aryl portion of -D-NR^{C}C(O)-aryl and the heterocyclyl portion of -D-heterocyclyl and the cycloalkyl portion of the -D-cycloalkyl may be optionally substituted with one or more R^{D}, and wherein the aryl or heteroaryl of the -D-aryl and the -D-heteroaryl may be optionally substituted with one or more R^{E}.

The linker of the degrader is joined to the Z¹ moiety at any position (provided that it has the correct valency and/or is chemically suitable). For example, where Z¹ comprises an aromatic or heteroaromatic ring, the linker may replace a hydrogen atom at any position on the ring.

Each D is independently a C₁₋₄alkylene optionally substituted with hydroxy, C₁₋₄hydroxyalkyl or heteroaryl.

Each R^{C} is independently hydrogen or C₁₋₃alkyl.

Each R^{D} is independently halo, hydroxy, C₁₋₃hydroxyalkyl, C₁₋₃alkyl, C₁₋₃haloalkyl, C₁₋₃alkoxy, cyano, -Q-phenyl, -Q-phenylSO₂F, —NHC(O)phenyl, —NHC(O)phenylSO₂F, C₁₋₃alkyl substituted pyrazolyl, araC₁₋₃alkyl-, tert-butyldimethylsilyloxyCH₂—, —N(R^{C})₂, (C₁₋₃alkoxy)C₁₋₃alkyl-, (C₁₋₃alkyl)C(O), oxo, (C₁₋₃haloalkyl)C(O)—, —SO₂F, (C₁₋₃alkoxy)C₁₋₃alkoxy, —CH₂OC(O)N(R^{C})₂, —CH₂NHC(O)OC₁₋₆alkyl, —CH₂NHC(O)N(R^{C})₂, —CH₂NHC(O)C₁₋₆alkyl, —CH₂(pyrazolyl), —CH₂NHSO₂C₁₋₆alkyl, —CH₂OC(O)heterocyclyl, —OC(O)N(R^{C})₂, —OC(O)NH(C₁₋₃alkyl)O(C₁₋₃alkyl), —OC(O)NH(C₁₋₃alkyl)O(C₁₋₃alkyl)phenyl(C₁₋₃alkyl)N(CH₃)₂, —OC(O)NH(C₁₋₃alkyl)O(C₁₋₃alkyl)phenyl or —OC(O)heterocyclyl, —CH₂heterocyclyl, wherein the phenyl of —NHC(O)phenyl or —OC(O)NH(C₁₋₃alkyl)O(C₁₋₃alkyl)phenyl is optionally substituted with —C(O)H or OH and wherein the heterocyclyl of —CH₂heterocyclyl is optionally substituted with oxo.

Q is a bond or O.

Each R^{E} is independently halogen, hydroxy, HC(O)—, C₁₋₄alkyl, C₁₋₄alkoxy, C₁₋₄haloalkyl, C₁₋₄hydroxyalkyl, or —N(R^{C})₂.

In some examples, Z¹ is as defined in (i).

In such examples:
L₁ may be O or substituted or unsubstituted C₂₋₃ alkynyl; and
Z¹ may be cyanoalkyl, alkylcarbonylaminoalkyl, alkylaminocarbonyl, alkylaminoalkyl, C₃₋₆cycloalkyl, a 5- to 6-membered saturated ring which contains at least one heteroatom selected from the group consisting of N, S, and O, an 8- to 10-membered partially unsaturated ring which contains at least one heteroatom selected from the group consisting of N, S, and O; wherein
the ring in Z¹ may be substituted by halo, hydroxy, C₁₋₆alkyl, C₁₋₃alkoxy, C₁₋₃hydroxyalkyl, C₁₋₃methoxyalkyl, a substituted or unsubstituted 5- to 6-membered saturated ring which contains at least one heteroatom selected from the group consisting of N, S, and O, and which may be substituted by C₁₋₃alkyl, alkylcarbonylalkyl, hydroxyalkyl, dialkylamino, dialkylaminoalkyl, alkoxyalkyl, or cyanoalkyl.

Sometimes, when L₁ is O, m1 is 0 or 1, and when L₁ is C₂₋₃alkynyl, m1 is 1 and Z¹ is dimethylaminocarbonyl or dimethylaminomethyl.

In some examples:
L₁ is O;
m1 is 0 or 1; and
Z¹ is a C₃₋₆cycloalkyl, a 5- to 6-membered saturated ring which contains at least one heteroatom selected from the group consisting of N, S, and O, an 8- to 10-membered partially saturated ring which contains at least one heteroatom selected from the group consisting of N, S, and O; wherein the ring in Z¹ may be substituted with halo, hydroxy, C₁₋₆alkyl, C₁₋₃alkoxy, C₂₋₃alkynyl, alkylcarbonylalkyl, hydroxyalkyl, dialkylamino, dialkylaminoalkyl, alkoxyalkyl, cyanoalkyl or C₁₋₆alkyl which is substituted with a 5- to 6-membered saturated ring which contains at least one heteroatom selected from the group consisting of N, S, and O and which may be further substituted with a halo.

In some examples:
L₁ is O;
m1 is 1; and
Z¹ is a C₃₋₆cycloalkyl or a 5- to 6-membered saturated ring which contains at least one heteroatom selected from the group consisting of N, S, and, O; wherein the ring in Z¹ may be substituted with a halo, hydroxy, cyano, C₁₋₆alkyl, C₁₋₃alkoxy, alkylcarbonylalkyl, hydroxyalkyl, dialkylamino, dialkylaminoalkyl, alkoxyalkyl, cyanoalkyl, or C₁₋₆alkyl which is substituted by a 5 to 6-membered saturated ring which contains at least one heteroatom selected from the group consisting of N, S, and O and which may be further substituted with a halo.

In some examples Z¹ is:
(i) cyclobutane, cyclopropane, piperidine, morpholine, piperazine, isoindoline or 1,2,3,4-tetrahydroisoquinoline, and may be substituted with a halo, hydroxy, cyano, C₁₋₆alkyl, or C₁₋₃alkoxy; or
(ii) alkylcarbonylalkyl, hydroxyalkyl, dialkylamino, dialkylaminoalkyl, alkoxyalkyl, cyanoalky or C₁₋₆alkyl which is substituted with a 5- to 6-membered saturated ring which contains at least one heteroatom selected from the group consisting of N and O and which may be further substituted with a halo.

In some examples, Z¹ is cyclobutane, cyclopropane, piperidine, morpholine, piperazine, isoindoline, or 1,2,3,4-tetrahydroisoquinoline which may be substituted with a halo, hydroxy, C₁₋₃alkoxy, methyl, ethyl, isopropanyl, ethylcalbonylmethyl, hydroxyethyl, dimethylamino, dimethylaminomethyl, methoxyethyl, cyanomethyl, morpholylmethyl, or 3-fluoropyrrolidinylmethyl.

In some examples, Z¹ is as defined in (ii).

In some cases, L₁ is O, m1 is 0 and Z¹ is C₁₋₆alkyl (such as methyl, ethyl, isopropyl or isobutyl) or -D-heterocyclyl optionally substituted with one or more R^{D} (as defined above with respect to formulae (KRAS1) and (KRAS3)).

Sometimes, D is methylene and the heterocyclyl is selected from the group consisting of hexahydro-1H-pyrrolizinyl, hexahydro-3H-pyrrolizin-3-one, hexahydro-1H-pyrrolo[2,1-c][1,4]oxazinyl, octahydroindolizinyl, hexahydropyrrolizine 4(1H)-oxide, azetidinyl, pyrrolidinyl, pyrrolidin-2-one, oxetanyl, piperidinyl, 1-azabicyclo[2.2.1]heptanyl, morpholinyl, oxa-5-azabicyclo[2.2.1]heptan-5-yl, thiopyranyl, 6-oxa-2-2λ²-azaspiro[3.4]octanyl, 7-oxa-2λ²-azaspiro[3.5]nonanyl, 2′,3′-dihydrospiro[cyclopropane-1, 1′-indenyl], (2S)-1-azabicyclo[2.2.1]heptan-2-yl, and tetrahydrofuranyl, each optionally substituted with one or more R^{D}.

Sometimes, D is methylene and the heterocyclyl is hexahydro-1H-pyrrolizinyl, optionally substituted with one or more R^{D}.

In some cases, the heterocyclyl is hexahydro-1H-pyrrolizinyl substituted with one or more R^{D} independently selected from the group consisting of halo (such as fluoro), hydroxy, C₁₋₃hydroxyalkyl, C₁₋₃haloalkyl, C₁₋₃alkyl, C₁₋₃alkoxy, phenyl or pyrazolyl.

Sometimes, the heterocyclyl is hexahydro-1H-pyrrolizinyl substituted with three R^{D} groups, one of which is halo (such as fluoro), hydroxy, C₁₋₃hydroxyalkyl, C₁₋₃haloalkyl, C₁₋₃alkyl, C₁₋₃alkoxy, phenyl or pyrazoly, and two of which are independently C₁₋₃alkyl.

Sometimes, the heterocyclyl is azetidinyl substituted with a C₁₋₃alkyl.

In some cases, the heterocyclyl is pyrrolidinyl substituted with any one moiety selected from the group consisting of hydroxalkyl, haloalkyl, C₁₋₃alkyl, alkoxy, araC₁₋₃alkyl, -Q-phenyl and —NHC(O)phenyl, and wherein the aryl portion of the araC₁₋₃alkyl or the phenyl portion of the —Q-phenyl and —NHC(O)phenyl are each optionally substituted with one or more R^{D}, such as SO₂F.

The heterocyclyl may be pyrrolidinyl substituted with two groups, wherein the first is a C₁₋₃alkyl and the second is a C₁₋₃alkoxy or a halo.

Sometimes, the heterocyclyl is pyrrolidin-2-one substituted with a C₁₋₃alkyl.

In some cases, the heterocyclyl is piperidinyl substituted with any one of the group consisting of acetyl, (C₁₋₃alkoxy)C₁₋₃alkoxy, or —C(O)CH₂Cl.

Often, L₁ is O, m1 is 0, D is ethylene or propylene and the heterocyclyl is morpholinyl or oxa-5-azabicyclo[2.2.1]heptan-5-yl.

Sometimes, L₁ is O, m1 is 0 and Z¹ is -D-heteroaryl, wherein the heteroaryl portion is optionally substituted with one or more R^{E}. D may be methylene or ethylene and the heteroaryl may be pyridyl, pyrazolyl, imidazolyl, triazolyl, 4,5,6,7-tetrahydro-1H-indazolyl, benzimidazolyl, imidazo[1,2-a]pyridinyl, or pyrimidinyl, each optionally substituted with one or more R^{E}. R^{E} may be one or more independently selected from the group consisting of halo, C₁₋₄alkyl, —N(R^{C})₂, or C₁₋₄alkoxy. For example, the heteroaryl may be pyrazolyl substituted with C₁₋₄alkyl or —N(R^{C})₂. Alternatively, the heteroaryl may be imidazolyl substituted with any one moiety selected from the group consisting of C₁₋₄alkyl, C₁₋₄haloalkyl, and C₁₋₄hydroxyalkyl. As another alternative, the heteroaryl is triazolyl substituted with C₁₋₄alkyl.

In some cases, L¹ is O, m1 is 0 and Z¹ is -D-aryl, wherein the aryl portion is optionally substituted with one or more R^{E} (as defined above with respect to formulae (KRAS1) and (KRAS3)).

In other cases, L¹ is O, m1 is 0 and Z¹ is -D-cycloalkyl, wherein the cycloalkyl portion is optionally substituted with one or more R^{E} (as defined above with respect to formulae (KRAS1) and (KRAS3)).

Sometimes, L¹ is O, m1 is 0 and Z¹ is -D-N(R^{C})₂. For example, D may be ethylene and each R^{C} may be independently selected from C₁₋₃alkyl.

In some cases, L¹ is O, m1 is 0 and Z¹ is -D-NC(═NH)—NH₂. For example, D may be ethylene or propylene.

In other cases, L¹ is O, m1 is 0 and Z¹ is -D-C₁₋₆haloalkyl. Alternatively, L¹ may be O, m1 may be 0 and Z¹ may be -D-OR^{C}. As another alternative, L¹ may be O, m1 may be 0 and Z¹ may be -D —(CH₂OR^{C}) (CH₂),OR^{C}. Otherwise, L¹ may be O, m1 may be 0 and R² may be -D-NR^{C}C(O)-aryl.

Moiety —(C(X¹)ₙ₁Y¹ of Formula (KRAS1):

In some cases, X¹ is O. Sometimes, n1 is 0.

Y¹ is an optionally substituted moiety selected from the group consisting of a 6- to 10-membered aromatic hydrocarbyl ring and a 6- to 10-membered unsaturated monocyclic or bicyclic ring, which contains at least one heteroatom selected from the group consisting of N, S and O. For example, Y¹ may be an optionally substituted 6- to 10-membered aromatic hydrocarbyl ring, such as an optionally substituted moiety selected from the group consisting of naphthyl, phenyl, 1,2,3,4-tetrahydronaphthalenyl and 2,3-dihydro-1H-indenyl. Typically, Y¹ is a substituted naphthyl, for example a naphthyl substituted with one or more substituents selected from the group consisting of hydroxy, ethynyl, halo (such as chloro or fluoro), C₁₋₄alkyl and C₁₋₄alkoxy.

In some examples, Y¹ is an 8- to 10-membered unsaturated bicyclic ring which contains at least one heteroatom selected from the group consisting of N and S, or a 6- to 10-membered aromatic hydrocarbon ring; wherein the ring may be substituted with one or substituents selected from the group consisting of halo, hydroxy, amino, C₁₋₆alkyl, C₂₋₃alkenyl, C₂₋₃alkynyl and 5- to 6-membered unsaturated monocyclic heterocyclyl which contains one or more heteroatoms selected from the group consisting of N, S and O.

In some examples, Y¹ is selected from the group consisting of benzene, naphthalene, benzo[b]thiophene, thieno[3,2-b]pyridine, isoquinoline, indole, and indazole, each of which is optionally substituted with one or more substituents selected from the group consisting of halo, hydroxy, amino, C₁₋₆alkyl, C₂₋₃alkenyl, C₂₋₃alkynyl and thiophenyl.

In some cases, Y¹ is an optionally substituted heteroaryl such as an optionally substituted moiety selected from the group consisting of isoquinolinyl, indazolyl, or benzo[d][1,3]dioxolyl. For example, Y¹ may be isoquinolinyl substituted with a halo or C₂₋₄alkynyl. Alternatively, Y¹ may be indazolyl substituted with a chloro or a C₁₋₃alkyl. As another alternative, Y¹ may be benzo[d][1,3]dioxolyl substituted with two halo.

In some cases, Y¹ is of formula (KRAS1b):

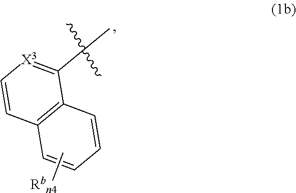

wherein:
X³ is any one selected from the group consisting of CH, CR^{b} and N;
n4 is 0 to 4; and
R^{b} is the optional substituent, described above.

The wavy lines on formula (1b) is shown over the bond that forms the link with the parent structure (as shown in formula (1).

Often, n4 is 2 or 1 (such as 2). Typically, X³ is CH or CR^{b}.

R^{b} may be any one or more substituents selected from the group consisting of hydroxy, C₂₋₄alkynyl (such as ethynyl), halo (such as chloro or fluoro), C₁₋₄alkoxy (such as methoxy), C₁₋₄alkyl, C₂₋₄haloalkynyl (such as haloethynyl), C₁₋₄haloalkyl, C₂₋₄alkenyl (such as ethenyl), C₂₋₄haloalkenyl, C₁₋₄haloalkoxy (such as halomethoxy), C₃₋₄cycloalkyl (such as cyclopropyl), C₁₋₄alkenylol and amino. For example, R^{b} may be any one or more substituents selected from the group consisting of hydroxy, C₂₋₄alkynyl (such as ethynyl), halo (such as chloro or fluoro), C₁₋₄alkoxy (such as methoxy), C₁₋₄alkyl, C₂₋₄haloalkynyl (such as haloethynyl) and C₁₋₄haloalkyl.

In some examples, R^{b} (or the optional substituent) is any one or more substituents selected from the group consisting of halo, cyano, hydroxy, C₁₋₄alkyl, —S—C₁₋₃alkyl, C₂₋₄alkenyl, C₂₋₄alkynyl, C₂₋₄hydroxyalkynyl, C₁₋₃cyanoalkyl, triazolyl, C₁₋₃haloalkyl, —O—C₁₋₃haloalkyl, —S—C₁₋₃haloalkyl, C₁₋₃alkoxy, hydroxyC₁₋₃alkyl, —CH₂C(O)N(R⁵)₂, —C₃₋₄alkynyl(NR⁵)₂, —N(R⁵)₂, deuteroC₂₋₄alkynyl, (C₁₋₃alkoxy)haloC₁₋₃alkyl-, and C₃₋₆cycloalkyl wherein said C₃₋₆cycloalkyl is optionally substituted with halo or C₁₋₃alkyl.

In some examples:
A¹ is of the following formula:

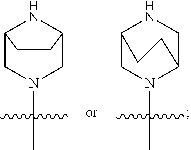 [SMILES: *N1CC2CCC(C1)N2 ; *N1CC2CCC1CN2]

B¹ is benzene, piperidine, or pyrrolidine each of which are optionally substituted with a halo or C₁₋₆alkyl; wherein when B¹ is pyrrolidine, n1 is 1 and X¹ is O, and when B¹ is not pyrrolidine,
n1 is 0;
Y¹ is naphthalenyl which may be substituted with a halo, hydroxy, C₁₋₆alkyl, C₂₋₃alkenyl, or C₂₋₃alkynyl;
L¹ is O;
m1 is 1; and
Z¹ is cyclobutanyl, cyclopropanyl, piperidinyl, morpholinyl, piperazinyl, isoindolinyl, or 1,2,3,4-tetrahydroisoquinolinyl, each of which is optionally substituted with a halo, hydroxy, C₁₋₃alkoxy, methyl, ethyl, isopropanyl, ethylcalbonylmethyl, hydroxyethyl, dimethylamino, dimethylaminomethyl, alkoxyalkyl, cyanomethyl, morpholinylmethyl, or 3-fluoropyrrolidinemethyl.
   CBP and/or p300 Inhibitors

In some instances, the target protein binding ligand may be derived from a CBP and/or p300 inhibitor. For example, the target protein binding ligand may have the following structure:

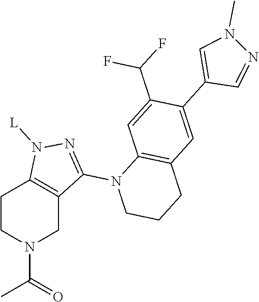

where L shows the position of attachment of the linker. Again, the present disclosure also encompasses joining or coupling to the linker at any other chemically suitable position on this target protein binding ligand.

In some examples, the target protein binding ligand may be represented by formula (C1):

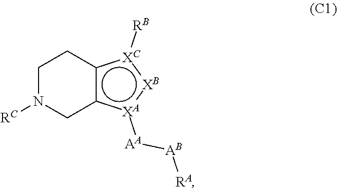

wherein:
the linker moiety of the bivalent compound is attached to R^{B} or R^{A};
X^{A} and X^{C} are independently selected from C and N, with the proviso that at least one of X^{A} and X^{C} is C and only one or fewer of X^{A} and X^{C} is N;
X^{B} is selected from CR^{XB1}, O, and NR^{XB2}, wherein R^{XB1} is any one substituent selected from the group consisting of H, optionally substituted C₁₋₈alkyl, and optionally substituted 3-10 membered carbocyclyl; and wherein R^{XB2} is absent or any one substituent selected from the group consisting of H, optionally substituted C₁₋₈alkyl, and optionally substituted 3-10 membered carbocyclyl;
A^{A} is absent or is selected from the group consisting of, CR^{AA1}R^{AA2}, CO, O, S, SO, SO₂, and NR^{AA1}, wherein R^{AA1} and R^{AA2} are each independently selected from the group consisting of hydrogen, halo, hydroxy, cyano, nitro, optionally substituted C₁₋₈alkyl, optionally substituted C₂₋₈alkenyl, optionally substituted C₂₋₈alkynyl, optionally substituted C₁₋₈alkoxy, optionally substituted C₁₋₈alkoxyC₁₋₈alkyl, optionally substituted C₁₋₈alkylamino, optionally substituted C₁₋₈alkylaminoC₁₋₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl;
A^{B} and R^{AA1}, A^{B} and R^{AA2}, and/or R^{AA1} and R^{AA2} together with the atom to which they are connected form an optionally substituted 3-20 membered cycloalkyl or heterocyclyl ring (such as a 4-20 membered heterocyclyl ring);
A^{B} is any one substituent selected from the group consisting of aryl, heteroaryl, bicyclic aryl, bicyclic heteroaryl, tricyclic aryl and tricyclic heteroaryl, wherein each substituent is substituted with R^{A} and is optionally substituted with one or more substituents independently selected from the group consisting of hydrogen, halo, oxo, CN, NO₂, OR^{AB1}, SR^{AB1}, NR^{AB1}R^{AB2}, OCOR^{AB1}, OCO₂R^{AB1}, OCONR^{AB1}R^{AB2}, COR^{AB1}, CO₂R^{AB1}, CONR^{AB1}R^{AB2}, SOR^{AB1}, SO₂R^{AB1}, SO₂NR^{AB1}R^{AB2}NR^{AB3}CO₂R^{AB1}, NR^{AB3}COR^{AB1}, NR^{AB3}C(O)NR^{AB1}R^{AB2}, NR^{AB3}SOR^{AB1} NR^{AB3}SO₂R^{AB1}, NR^{AB3}SO₂NR^{AB1}R^{AB2}, optionally substituted C₁₋₈alkyl, optionally substituted C₂₋₈alkenyl, optionally substituted C₂₋₈alkynyl, optionally substituted C₁₋₈alkoxy, optionally substituted C₁₋₈alkoxyC₁₋₈alkyl, optionally substituted C₁₋₈alkylamino, optionally substituted C₁₋₈alkylaminoC₁₋₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein
R^{AB1}, R^{AB2} and R^{AB3} are each independently selected from hydrogen, optionally substituted C₁₋₈alkyl, optionally substituted C₂₋₈alkenyl, optionally substituted C₂₋₈alkynyl, optionally substituted C₁₋₈alkoxyC₁₋₈alkyl, optionally substituted C₁₋₈alkylaminoC₁₋₈alkyl, optionally substituted 3-10 membered carbocyclyl, optionally substituted 4-10 membered heterocyclyl, optionally substituted aryl, and optionally substituted heteroaryl, or
R^{AB1} and R^{AB2} or R^{AB1} and R^{AB3} together with the atom to which they are connected form a 4-20 membered heterocyclyl ring;
R^{A} is absent or is selected from hydrogen, halo, CN, NO₂, OR^{A1}, S R^{A1}, NR^{A1}R^{A2}, OCOR^{A1}, OCOR^{A1}, OCONR^{A1}R^{A2}, COR^{A1}, CO₂R^{A1}, CONR^{A1}R^{A2}, SOR^{A1}, SO₂R^{A1} SO₂NR^{A1}R^{A2}, NR^{A3}CO₂R^{A1}, NR^{A3}COR^{A1}, NR^{A3}C(O)NR^{A1}R^{A2}, NR^{A3}SOR^{A1}, NR^{A3}SO₂R^{A1} NR^{A3}SONR^{A1}R^{A2}, optionally substituted C₁₋₈alkyl, optionally substituted C₂₋₈alkenyl, optionally substituted C₂₋₈alkynyl, optionally substituted C₁₋₈alkoxy, optionally substituted C₁₋₈alkoxyC₁₋₈alkyl, optionally substituted C₁₋₈alkylamino, optionally substituted C₁₋₈alkylaminoC₁₋₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, wherein
R^{A1}, R^{A2}, and R^{A3} are independently selected from hydrogen, optionally substituted C₁-C₈alkyl, optionally substituted C₂₋₈alkenyl, optionally substituted C₂₋₈alkynyl, optionally substituted C₁₋₈alkoxyC₁₋₈alkyl, optionally substituted C₁₋₈alkylaminoC₁₋₈alkyl, optionally substituted 3-10 membered carbocyclyl, optionally substituted 4-10 membered heterocyclyl, optionally substituted aryl, and optionally substituted heteroaryl, or
R^{A1} and R^{A2} or R^{A1} and R^{A3} together with the atom to which they are connected form a 3-20 membered cycloalkyl or 4-20 membered heterocyclyl ring;
R^{B} is absent or is selected from the group consisting of R^{B1}O, R^{B1}S, R^{B1}NR^{B2}, R^{B1}OC(O), R^{B1}OC(O)O, R^{B1}OCONR^{B2}, R^{B1}C(O), R^{B1}C(O)O, R^{B1}CONR^{B2}, R^{B1}S(O), R^{B1}SO₂, R^{B1}SO₂NR^{B2}, R^{B1}NR^{B3}C(O)O, R^{B1}NR^{B3}C(O), R^{B1}NR^{B3}C(O)NR^{B2}, R^{B1}NR^{B3}S(O), R^{B1}N R^{B3}SO₂, R^{B1}NR^{B3}SO₂NR^{B2}, optionally substituted C₁₋₈alkylene, optionally substituted C₂₋₈alkenylene, optionally substituted C₂₋₈alkynylene, optionally substituted 3-10 membered carbocyclyl, optionally substituted 4-10 membered heterocyclyl, optionally substituted C₃₋₁₃fused cycloalkyl, optionally substituted C₃₋₁₃fused heterocyclyl, optionally substituted C₃₋₁₃bridged cycloalkyl, optionally substituted C₃₋₁₃bridged heterocyclyl, optionally substituted C₃₋₁₃spiro cycloalkyl, optionally substituted C₃₋₁₃spiro heterocyclyl, optionally substituted aryl, and optionally substituted heteroaryl, wherein

R^{B1} is absent, or is a bivalent moiety selected from the group consisting of optionally substituted C₁₋₈alkylene, optionally substituted C₂₋₈alkenylene, optionally substituted C₂₋₈₋alkynylene, optionally substituted C₁₋₈alkoxyC₁₋₈alkylene, optionally substituted C₁₋₈₋haloalkylene, optionally substituted C₁₋₈hydroxyalkylene, optionally substituted 3-10 membered carbocyclyl, optionally substituted 4-10 membered heterocyclyl, optionally substituted C₃₋₁₃fused cycloalkyl, optionally substituted C₃₋₁₃fused heterocyclyl, optionally substituted C₃₋₁₃bridged cycloalkyl, optionally substituted C₃₋₁₃bridged heterocyclyl, optionally substituted C₃₋₁₃spiro cycloalkyl, optionally substituted C₃₋₁₃spiro heterocyclyl, optionally substituted aryl, and optionally substituted heteroaryl;
R^{B2} and R^{B3} are each independently selected from the group consisting of optionally substituted C₁₋₈alkyl, optionally substituted C₂₋₈alkenyl, optionally substituted C₂₋₈alkynyl, optionally substituted C₁₋₈alkoxyC₁₋₈alkyl, optionally substituted C₁₋₈alkylaminoC₁₋₈alkyl, optionally substituted 3-10 membered carbocyclyl, optionally substituted 4-10 membered heterocyclyl, optionally substituted aryl, and optionally substituted heteroaryl, or
R^{B2} and R^{B3} together with the atom to which they are connected form a 3-20 membered cycloalkyl or 4-20 membered heterocyclyl ring; and
R^{C} is selected from hydrogen, COR^{C1}, CO₂R^{C1}, CONR^{C1}R^{C2}, SOR^{C1}, SO₂R^{C1}, SO₂NR^{C1}R^{C2}, optionally substituted C₁₋₆alkyl, optionally substituted C₂₋₆alkenyl, optionally substituted C₂₋₆alkynyl, optionally substituted 3-6 membered cycloalkyl, optionally substituted 4-6 membered heterocyclyl, optionally substituted aryl, and optionally substituted heteroaryl, wherein
R^{C1} and R^{C2} are each independently selected from the group consisting of hydrogen, optionally substituted C₁₋₆alkyl, optionally substituted C₂₋₆alkenyl, optionally substituted C₂-6alkynyl, optionally substituted 3-6 membered cycloalkyl, optionally substituted 4-6 membered heterocyclyl, optionally substituted aryl, and optionally substituted heteroaryl, or
R^{C1} and R^{C2} together with the atom to which they are connected form a 4-20 membered heterocyclyl ring.

The target protein binding ligand represented in formula (C1) may be appended to the linker L of the bifunctional molecule by way of a covalent bond between an atom on the target protein binding ligand (TBL) and an atom on the linker (L). The linker may be attached at any suitable position e.g. provided it has the correct valency and/or is chemically suitable.

By way of example, the linker of the bifunctional molecule may be covalently bonded to TBL as shown in formula (C1) by way of the R^{A} or R^{B} groups. For example, the linker may be covalently bonded to an atom on either of the R^{A} or R^{B} groups of formula (C1) at any position (provided that it has the correct valency and/or is chemically suitable) (e.g. by replacing a hydrogen atom).

In some examples, the linker is attached to R^{A}. In other examples, the linker is attached to R^{B}.

For the avoidance of doubt, when it is specified that the linker is attached to R^{A} and R^{A} is defined as absent, the linker is instead attached (e.g. covalently bonded) to A^{B}. Similarly, when it is specified that the linker is attached to R^{B} and R^{B} is defined as absent, the linker is instead attached (e.g. covalently bonded) to X^{C}.

In more specific examples, X^{A} is C and X^{B} and X^{C} are each N, i.e. the compound is of formula (C2):

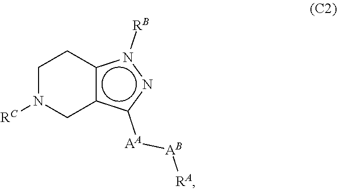

wherein the linker moiety of the bivalent compound is attached to R^{B} or R^{A} and A^{A} A^{B}, R^{A}, R^{B} and R^{C} are as defined above in respect of formula (C1).
   A^{A}-A^{B}-R^{A} Moiety of Formulae (C1 and C2)

In some examples, the A^{A}-A^{B}-R^{A} moiety is represented by formula (C3):

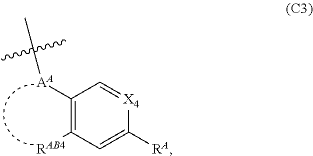

wherein:
A^{A} and R^{A} are as defined above for formula (C1);
X⁴ is selected from CR^{X41} and N, wherein R^{X41} is selected from hydrogen, halo, hydroxy, amino, cyano, nitro, optionally substituted C₁₋₆alkyl, optionally substituted C₂₋₆alkenyl, optionally substituted C₂₋₆alkynyl, optionally substituted C₁₋₆alkoxy, optionally substituted C₁₋₆alkylamino, optionally substituted 3-6 membered cycloalkyl, optionally substituted 3-6 membered cycloalkoxy, optionally substituted 3-6 membered cycloalkylamino, optionally substituted 4-6 membered heterocyclyl, optionally substituted aryl, and optionally substituted heteroaryl; and
R^{AB4} is absent, optionally forms a ring with A^{A}, or is any one moiety selected from the group consisting of hydrogen, halo, R^{AB5}NR^{AB6}, R^{AB5}OR^{AB6}, R^{AB5}SR^{AB6}R^{AB5}NR^{AB6}R^{AB7}, R^{AB5}OCOR^{AB6}, R^{AB5}OCO₂R^{AB6}, R^{AB5}OCONR^{AB6}R^{AB7}, R^{AB5}COR^{AB6}, R^{AB5}CO₂R^{AB6}, R^{AB5}CONR^{AB6}R^{AB7}, R^{AB5}SOR^{AB6}, R^{AB5}SO₂R^{AB6}R^{AB5}SO₂NR^{AB6}R^{AB7}, R^{AB5}NR^{AB8}CO₂R^{AB6}, R^{AB5}NR^{AB8}COR^{AB6}, R^{AB5}NR^{AB8}C(O)NR^{AB6}R^{AB7}, R^{AB5}NR^{AB8}SOR^{AB6}, R^{AB5}NR^{AB8}SO₂R^{AB6}, R^{AB5}NR^{AB8}SO₂NR^{AB6}R^{AB7}, optionally substituted C₁₋₈alkyl, optionally substituted C₁₋₈alkylene, optionally substituted C₂₋₈alkenyl, optionally substituted C₂₋₈alkenylene, optionally substituted C₂₋₈alkynyl, optionally substituted C₂₋₈alkynylene, optionally substituted 3-10 membered carbocyclyl, optionally substituted 4-10 membered heterocyclyl, optionally substituted aryl, and optionally substituted heteroaryl, wherein
R^{AB5} is absent or is a bivalent or trivalent moiety selected from optionally substituted C₁₋₈alkylene, optionally substituted C₂₋₈alkenylene, optionally substituted C₂₋₈alkynylene, optionally substituted C₁₋₈alkoxyC₁₋₈alkylene, optionally substituted C₁₋₈alkylaminoC₁₋₈alkylene, optionally substituted C₁₋₈haloalkylene, optionally substituted C₁₋₈hydroxyalkylene, optionally substituted 3-10 membered carbocyclyl, optionally substituted 4-10 membered heterocyclyl, optionally substituted aryl, and optionally substituted heteroaryl;
R^{AB6}R^{AB7} and R^{AB8} are each independently absent or selected from the group consisting of hydrogen, optionally substituted C₁₋₈alkyl, optionally substituted C₂₋₈alkenyl, optionally substituted C₂₋₈alkynyl, optionally substituted C₁₋₈alkoxyC₁₋₈alkyl, optionally substituted C₁₋₈alkylaminoC₁₋₈alkyl, optionally substituted 3-10 membered carbocyclyl, optionally substituted 4-10 membered heterocyclyl, optionally substituted aryl, and optionally substituted heteroaryl, or
R^{AB6} and R^{AB7}, or R^{AB6} and R^{AB8} together with the atom to which they are connected form a 3-20 membered cycloalkyl or heterocyclyl ring.

In some cases, A^{A} is absent.

In some examples, A^{B} -R^{A} is represented by formulae (C₄) or (C₅):

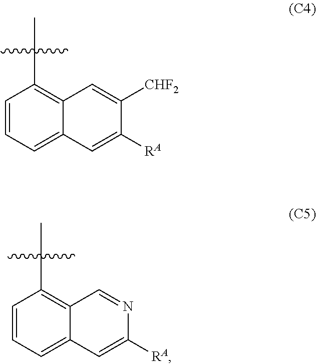

wherein R^{A} is as defined above. For the avoidance of doubt, where A^{A} is absent, the bond bisected with a wavy line is attached to the central ring motif of formulae (C1) or (C₂). Where A^{A} is present, the bond bisected with a wavy line is attached to A^{A}.

In some cases, A^{A} is NR^{AA1}, wherein R^{AA1} is selected from the group consisting of hydrogen, optionally substituted C₁₋₈alkyl, optionally substituted C₂₋₈alkenyl, optionally substituted C₂₋₈alkynyl, optionally substituted C₁₋₈alkoxyC₁₋₈alkyl, optionally substituted C₁₋₈alkylaminoC₁₋₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl

In some examples, A^{A}-A^{B}-R^{A} is represented by any one of formulae (C₄) to (C₈):

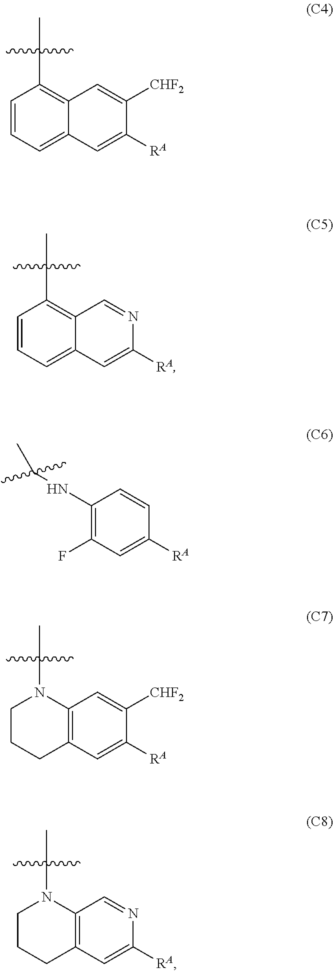

wherein R^{A} is as defined above.

In some examples, A^{A}-A^{B}-R^{A} is represented by any one of formulae (C6), (C7) and (C8), wherein R^{A} is as defined above for formula (C1).

In some examples, R^{A} is selected from the group consisting of optionally substituted 3-10 membered carbocyclyl, optionally substituted 4-10 membered heterocyclyl, optionally substituted aryl and optionally substituted heteroaryl. In some cases, R^{A} is aromatic. For example, R^{A} may be selected from optionally substituted aryl (such as phenyl) and optionally substituted heteroaryl (such as pyrazolyl or pyridinyl). Sometime, R^{A} is an optionally substituted heteroaryl. For example, R^{A} may be selected from optionally substituted pyrazolyl (such as N-methylpyrazolyl) and optionally substituted pyridinyl.

In more specific examples, A^{A}-A^{B}-R^{A} is represented by any one of formulae (C9) to (C12):

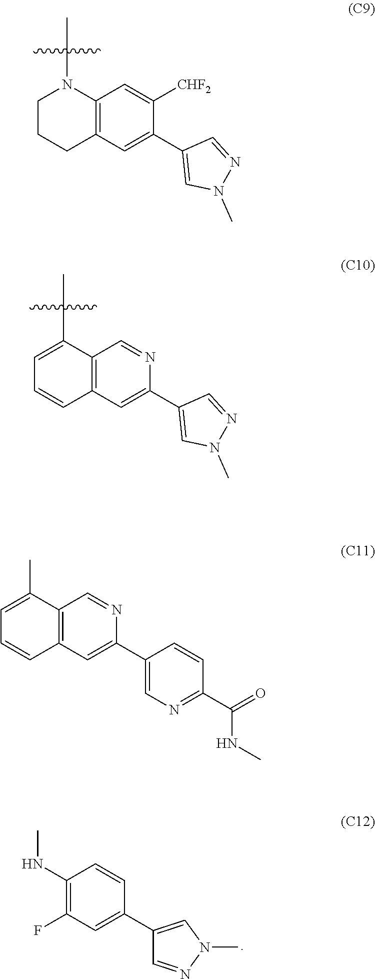 [SMILES: *N1CCCc2cc(-c3cnn(C)c3)c(C(F)F)cc21 ; *c1cccc2cc(-c3cnn(C)c3)ncc12 ; CNC(=O)c1ccc(-c2cc3cccc(C)c3cn2)cn1 ; CNc1ccc(-c2cnn(C)c2)cc1F]

In even more specific examples, A^{A}-A^{B}-R^{A} is represented by any one of formula (C9).

### R^{B} Moiety of any One of Formulae (C1) to (C12)

As described above, R^{B} is absent or is selected from the group consisting of R^{B1}O, R^{B1}S, R^{B1}NR^{B2}, R^{B1}OC(O), R^{B1}OC(O)O, R^{B1}OCONR^{B2}, R^{B1}C(O), R^{B1}C(O)O, R^{B1}CONR^{B2}, R^{B1}S(O), R^{B1}SO₂, R^{B1}SO₂NR^{B2}, R^{B1}NR^{B3}C(O)O, R^{B1}NR^{B3}C(O), R^{B1}NR^{B3}C(O)NR^{B2}, R^{B1}NR^{B3}S(O), R^{B1}N R^{B3}SO₂, R^{B1}NR^{B3}SO₂NR^{B2}, optionally substituted C₁₋₈alkylene, optionally substituted C₂₋₈alkenylene, optionally substituted C₂₋₈alkynylene, optionally substituted 3-10 membered carbocyclyl, optionally substituted 4-10 membered heterocyclyl, optionally substituted C₃₋₁₃fused cycloalkyl, optionally substituted C₃₋₁₃fused heterocyclyl, optionally substituted C₃₋₁₃bridged cycloalkyl, optionally substituted C₃₋₁₃bridged heterocyclyl, optionally substituted C₃₋₁₃spiro cycloalkyl, optionally substituted C₃₋₁₃spiro heterocyclyl, optionally substituted aryl, and optionally substituted heteroaryl. As stated above, in some examples, R^{B} is connected to the “linker” moiety of the bifunctional molecule.

In some examples, R^{B} is selected from optionally substituted C₁₋₈alkylene, optionally substituted 3-10 membered carbocyclyl, optionally substituted 4-8 membered heterocyclyl, optionally substituted aryl, and optionally substituted heteroaryl. Sometimes, R^{B} is optionally substituted 4-8 membered heterocyclyl. Sometimes, R^{B} is optionally substituted 4-8 membered N-heterocyclyl such as an optionally substituted 5-7 membered N-heterocyclyl. In specific examples, R^{B} is optionally substituted 6 membered N-heterocyclyl such as an optionally substituted piperidinyl. Sometimes, R^{B} is piperidinyl, wherein the piperidinyl is bonded to the linker via the nitrogen atom.

### R^{C} Moiety of any One of Formulae (C1) to (C12)

As described above, R^{C} is selected from hydrogen, COR^{C1}, CO₂R^{C1}, CONR^{C1}R^{C2}, SOR^{C1}, SO₂R^{C1}, SO₂NR^{C1}R^{C2}, optionally substituted C₁₋₆alkyl, optionally substituted C₂₋₆alkenyl, optionally substituted C₂₋₆alkynyl, optionally substituted 3-6 membered cycloalkyl, optionally substituted 4-6 membered heterocyclyl, optionally substituted aryl, and optionally substituted heteroaryl.

R^{C1} and R^{C2} are each independently selected from the group consisting of hydrogen, optionally substituted C₁₋₆alkyl, optionally substituted C₂₋₆alkenyl, optionally substituted C₂₋₆alkynyl, optionally substituted 3-6 membered cycloalkyl, optionally substituted 4-6 membered heterocyclyl, optionally substituted aryl, and optionally substituted heteroaryl, or R^{C1} and R^{C2} together with the atom to which they are connected form a 4-20 membered heterocyclyl ring.

In some examples, R^{C} is selected from the group consisting of COR^{C1} and CONR^{C1}R^{C2}, wherein R^{C1} and R^{C2} are as defined above. Sometimes, R^{C1} and R^{C2} are each independently selected from the group consisting of hydrogen and optionally substituted C₁₋₆alkyl. For example, R^{C1} and R^{C2} may each independently be selected from hydrogen and unsubstituted C₁₋₆alkyl (such as unsubstituted C₁₋₃alkyl). In some cases, R^{C1} is a C₁₋₆alkyl (such as a C₁₋₃alkyl, e.g. methyl) and R^{C2} is hydrogen. In some examples, R^{C} is selected from COMe and CONHMe.

### PARP

In other instances, the target protein binding ligand may be derived from a polymerase inhibitor, such as a PARP1 inhibitor. For example, the target protein binding ligand may have the following structure:

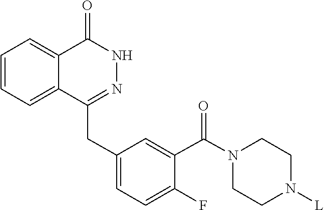

where L shows the position of attachment of the linker. The present disclosure also encompasses joining or coupling to the linker at any other chemically suitable position on this target protein binding ligand.

In other instances, the target protein binding ligand may be derived from a polymerase inhibitor, such as a POLQ inhibitor. For example, the target protein binding ligand may have the following structure:

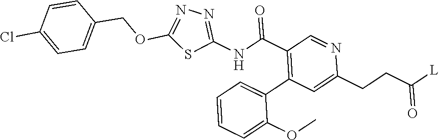

where L shows the position of attachment of the linker. The present disclosure also encompasses joining or coupling to the linker at any other chemically suitable position on this target protein binding ligand.

In other instances, the target protein binding ligand may be derived from a deubiquitinase inhibitor, such as a USP1 inhibitor. For example, the target protein binding ligand may have the following structure:

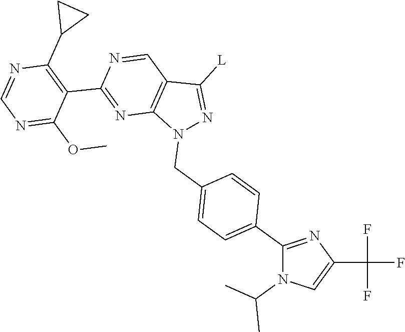

where L shows the position of attachment of the linker. The present disclosure also encompasses joining or coupling to the linker at any other chemically suitable position on this target protein binding ligand.

Representative examples of possible target protein binding ligand moieties for each of the various classes of target protein binding ligands are described below.

### SMARCA2/SMARCA4

In some instances, the target protein binding ligand may be derived from a SMARCA2 inhibitor. For example, the target protein binding ligand may have the structure of formula 1T:

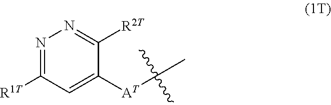

wherein the wavy line intersects the bond between the SMARCA2/SMARCA4 binder and the linker; and
wherein:
R^{1T} is hydrogen, halo, C₁-C₄ alkyl, C₂-C₄ alkenyl, C₁-C₄ alkoxy, hydroxy, —COOR^{at}, —CON(R^{at})₂, C₆-C₁₀ aryl or C₅-C₉ heteroaryl; wherein, the alkyl, alkenyl, alkoxy, aryl and heteroaryl are each optionally substituted with one or more groups independently selected from hydroxy, C₁-C₄ alkoxy, C₁-C₄ haloalkoxy, halo, C₁-C₄ alkyl, C₁-C₄ haloalkyl, amino, —COOR^{at} and —OCOR^{at}; wherein each R^{at} is independently selected from hydrogen and C₁-C₄ alkyl;
R^{2T} is —NR^{3T}R^{4T} or —OR^{3T};
A^{T} is absent or is selected from O, CR^{5T}R^{6T}, C₆-C₁₀ aryl optionally substituted with one or more R^{7T}, a three-to eight-membered heteroaryl optionally substituted with one or more R^{7T}, and a three-to eight-membered heterocycloalkyl optionally substituted with one or more R^{8T};
each R^{3T}, R^{4T}, R^{3T}, R^{4T}, R^{5T}, R^{6T}, and R^{7T} is independently selected from hydrogen, C₁-C₄ alkyl and C₁-C₄ haloalkyl;
each R^{8T} is independently selected from hydrogen, C₁-C₄ alkyl, C₁-C₄ haloalkyl and aryl; or
R^{3T} and R^{5T} together with the atoms to which they are attached form a five or six membered heterocycloalkyl or a five or six membered heteroaryl, wherein the heterocycloalkyl and the heteroaryl are optionally substituted with one or more groups independently selected from hydroxy, C₁-C₄ alkoxy, C₁-C₄ haloalkoxy, halo, C₁-C₄ alkyl, C₁-C₄ haloalkyl, amino, COOR^{at} and —OCOR^{at}; wherein each R^{at} is independently selected from hydrogen and C₁-C₄ alkyl; or
R^{3T} and one R^{8T} together with the atoms which they are attached to form a five or six membered heterocycloalkyl or a five or six membered heteroaryl, wherein the heterocycloalkyl and the heteroaryl are optionally substituted with one or more groups independently selected from hydroxy, C₁-C₄ alkoxy, C₁-C₄ haloalkoxy, halo, C₁-C₄ alkyl, C₁-C₄ haloalkyl, amino, —COOR^{at} and —OCOR^{at}; wherein each R^{at} is independently selected from hydrogen and C₁-C₄ alkyl.

In some embodiments, R^{1T} is C₆-C₁₀ aryl optionally substituted with one or more groups independently selected from hydroxy, C₁-C₄ alkoxy, C₁-C₄ haloalkoxy, halo, C₁-C₄ alkyl, C₁-C₄ haloalkyl, amino, —COOR^{at} and —OCOR^{at}; wherein each R^{at} is independently selected from hydrogen and C₁-C₄ alkyl.

In some embodiments, R^{1T} is C₆-C₁₀ aryl substituted with one or more groups independently selected from hydroxy, C₁-C₄ alkoxy, C₁-C₄ haloalkoxy, halo, C₁-C₄ alkyl, C₁-C₄ haloalkyl, and amino.

In some embodiments, R^{1T} is a group of the following structure:

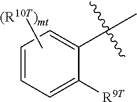

wherein:
mt is 0, 1, 2, 3, or 4;
R^{9T} is selected from hydroxy, C₁-C₄ alkoxy, C₁-C₄ haloalkoxy, halo, C₁-C₄ alkyl, C₁-C₄ haloalkyl, amino, —COOR^{at} and —OCOR^{at}; wherein each R^{at} is independently selected from hydrogen and C₁-C₄ alkyl; and
each R^{10T} is independently selected from hydroxy, C₁-C₄ alkoxy, C₁-C₄ haloalkoxy, halo, C₁-C₄ alkyl, C₁-C₄ haloalkyl, amino, —COOR^{at} and —OCOR^{at}; wherein each R^{at} is independently selected from hydrogen and C₁-C₄ alkyl,
wherein the wavy line intersects the bond between R^{1T} and the rest of the molecule.

In some embodiments, R^{1T} is a group of the following structure:

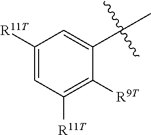

wherein:
R^{9T} is selected from hydroxy, C₁-C₄ alkoxy, C₁-C₄ haloalkoxy, halo, C₁-C₄ alkyl, C₁-C₄ haloalkyl, amino, —COOR^{at} and —OCOR^{at}; wherein each R^{at} is independently selected from hydrogen and C₁-C₄ alkyl; and
each R^{11T} is independently selected from hydrogen, hydroxy, C₁-C₄ alkoxy, C₁-C₄ haloalkoxy, halo, C₁-C₄ alkyl, C₁-C₄ haloalkyl, amino, —COOR^{at} and —OCOR^{at}; wherein each R^{at} is independently selected from hydrogen and C₁-C₄ alkyl,
wherein the wavy line intersects the bond between R^{1T} and the rest of the molecule.

In some embodiments, R_{1T} is a group of the following structure:

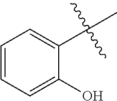 [SMILES: *c1ccccc1O]

wherein the wavy line intersects the bond between R^{1T} and the rest of the molecule.

In some embodiments, R^{2T} is —NR^{3T}R^{4T}.

In some embodiments, A^{T} is selected from CR^{5T}R^{6T}, a three-to eight-membered heteroaryl optionally substituted with one or more R^{7T}, and a three-to eight-membered heterocycloalkyl optionally substituted with one or more R^{8T}.

In some embodiments, A^{1T} is selected from a three-to eight-membered heteroaryl and a three-to eight-membered heterocycloalkyl.

In some embodiments, A^{1T} is selected from piperidinyl, piperazinyl, pyridyl, pyrazinyl, pyrrolidinyl, pyrryl, pyrazolidinyl, pyrazolyl, imidazolyl, imidazolidinyl, and diazabicyclo[3.2.1]octanyl.

In some embodiments, A^{1T} is selected from piperidinyl, pyrazolyl, and diazabicyclo[3.2.1]octanyl.

In some embodiments, mt is 0, 1 or 2.

In some embodiments, R^{3T} is selected from hydrogen, C₁-C₄ alkyl and C₁-C₄ haloalkyl.

In some embodiments, R^{3T} is selected from hydrogen and C₁-C₄ alkyl.

In some embodiments, R^{3T} is hydrogen.

In some embodiments, R^{4T} is selected from hydrogen, C₁-C₄ alkyl and C₁-C₄ haloalkyl.

In some embodiments, R^{4T} is selected from hydrogen and C₁-C₄ alkyl.

In some embodiments, R^{4T} is hydrogen.

In some embodiments, R^{5T} is selected from hydrogen, C₁-C₄ alkyl and C₁-C₄ haloalkyl.

In some embodiments, R^{5T} is selected from hydrogen and C₁-C₄ alkyl.

In some embodiments, R^{5T} is hydrogen.

In some embodiments, R^{3T} and R^{5T} together with the atoms which they are attached to form a five or six membered heterocycloalkyl or a five or six membered heteroaryl.

In some embodiments, R^{6T} is selected from hydrogen, C₁-C₄ alkyl and C₁-C₄ haloalkyl.

In some embodiments, R^{6T} is selected from hydrogen and C₁-C₄ alkyl.

In some embodiments, R^{6T} is hydrogen.

In some embodiments, R^{7T} is selected from hydrogen, C₁-C₄ alkyl and C₁-C₄ haloalkyl.

In some embodiments, R^{7T} is selected from hydrogen and C₁-C₄ alkyl.

In some embodiments, R^{7T} is hydrogen.

In some embodiments, R^{8T} is selected from hydrogen, C₁-C₄ alkyl and C₁-C₄ haloalkyl.

In some embodiments, R^{8T} is selected from hydrogen and C₁-C₄ alkyl.

In some embodiments, R^{8T} is hydrogen.

In some embodiments, R^{3T} and one R^{8T} together with the atoms which they are attached to form a five or six membered heterocycloalkyl or a five or six membered heteroaryl.

In some embodiments, R^{9T} is selected from hydroxy, C₁-C₄ alkoxy, C₁-C₄ haloalkoxy, halo, C₁-C₄ alkyl, C₁-C₄ haloalkyl, and amino.

In some embodiments, R^{9T} is selected from hydroxy, C₁-C₄ alkoxy, C₁-C₄ haloalkoxy, and amino.

In some embodiments, R^{9T} is selected from hydroxy and amino.

In some embodiments, R^{9T} is hydroxy.

In some embodiments, each R^{10T} is independently selected from hydroxy, C₁-C₄ alkoxy, C₁-C₄ haloalkoxy, halo, C₁-C₄ alkyl, C₁-C₄ haloalkyl, and amino.

In some embodiments, each R^{10T} is independently selected from halo, C₁-C₄ alkyl, and C₁-C₄ haloalkyl.

In some embodiments, each R^{10T} is independently selected from halo.

In some embodiments, each R^{11T} is independently selected from hydrogen, hydroxy, C₁-C₄ alkoxy, C₁-C₄ haloalkoxy, halo, C₁-C₄ alkyl, C₁-C₄ haloalkyl, and amino.

In some embodiments, each R^{11T} is independently selected from hydrogen, halo, C₁-C₄ alkyl, and C₁-C₄ haloalkyl.

In some embodiments, each R^{11T} is independently selected from hydrogen, halo, and C₁-C₄ alkyl.

In some embodiments, each R^{11T} is independently selected from hydrogen and halo.

In some embodiments, the SMARCA binder is of formula 2T:

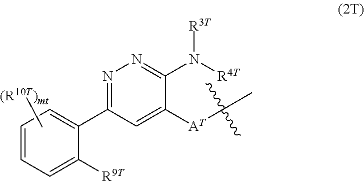

wherein the wavy line intersects the bond between the SMARCA2 binder and the linker; and
wherein:
mt is 0, 1, 2, 3, or 4;
A^{1T} is selected from a three-to eight-membered heteroaryl optionally substituted with one or more R^{7T}, and a three-to eight-membered heterocycloalkyl optionally substituted with one or more R^{8T};
each R^{3T}, R^{4T}, R^{7T} and R^{8T} is independently selected from hydrogen, C₁-C₄ alkyl and C₁-C₄ haloalkyl;
R^{9T} is selected from hydroxy, C₁-C₄ alkoxy, C₁-C₄ haloalkoxy, and amino; and each R^{10T} is independently selected from hydroxy, C₁-C₄ alkoxy, C₁-C₄ haloalkoxy, halo, C₁-C₄ alkyl, C₁-C₄ haloalkyl, and amino.

In some embodiments of the SMARCA binder of formula 2T,
mt is 0, 1 or 2;
A^{1T} is selected from a three-to eight-membered heteroaryl and a three-to eight-membered heterocycloalkyl;
R^{3T} and R^{4T} are both hydrogen;
R^{9T} is hydroxy, C₁-C₄ alkoxy, C₁-C₄ haloalkoxy, and amino;
each R^{10T} is independently selected from hydroxy, C₁-C₄ alkoxy, C₁-C₄ haloalkoxy, halo, C₁-C₄ alkyl and C₁-C₄ haloalkyl.

In some embodiments of the SMARCA binder of formula 2T,
mt is 0, 1 or 2;
A^{1T} is selected from piperidinyl, piperazinyl, pyridyl, pyrazinyl, pyrrolidinyl, pyrryl, pyrazolidinyl, pyrazolyl, imidazolyl, imidazolidinyl, and diazabicyclo[3.2.1]octanyl;
R^{3T} and R^{4T} are both hydrogen;
R^{9T} is hydroxy, C₁-C₄ alkoxy, C₁-C₄ haloalkoxy, and amino;
each R^{10T} is independently selected from hydroxy, C₁-C₄ alkoxy, C₁-C₄ haloalkoxy, halo, C₁-C₄ alkyl and C₁-C₄ haloalkyl.

In some embodiments of the SMARCA binder of formula 2T,
mt is 0, 1 or 2;
A^{1T} is selected from piperidinyl, pyrazolyl, and diazabicyclo[3.2.1]octanyl;
R^{3T} and R^{4T} are both hydrogen;
R^{9T} is hydroxy, C₁-C₄ alkoxy, C₁-C₄ haloalkoxy, and amino;
each R^{10T} is independently selected from hydroxy, C₁-C₄ alkoxy, C₁-C₄ haloalkoxy, halo, C₁-C₄ alkyl and C₁-C₄ haloalkyl.

In some embodiments, the SMARCA binder is of formula 3T:

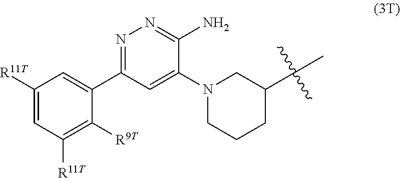

wherein the wavy line intersects the bond between the SMARCA2 binder and the linker; and

wherein:

R^{9T} is hydroxy or amino; and

each R^{11T} is independently selected from H, and halo.

In some embodiments, the SMARCA2 binder is of formula 4T:

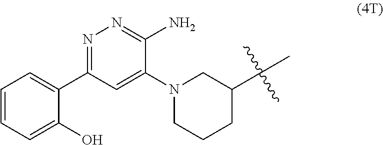 [SMILES: *C1CCCN(c2cc(-c3ccccc3O)nnc2N)C1]

wherein the wavy line intersects the bond between the SMARCA2 binder and the linker.

In some embodiments, the SMARCA2 binder is of the formula 5T:

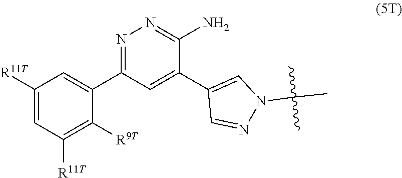

wherein the wavy line intersects the bond between the SMARCA2 binder and the linker; and
wherein:
R^{9T} is hydroxy or amino; and
each R^{11T} is independently selected from H, and halo.

In some embodiments, the SMARCA2 binder is of the formula 6T:

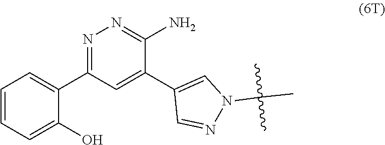 [SMILES: *n1cc(-c2cc(-c3ccccc3O)nnc2N)cn1]

wherein the wavy line intersects the bond between the SMARCA2 binder and the linker.

In some embodiments, the SMARCA2 binder is of the formula 7T:

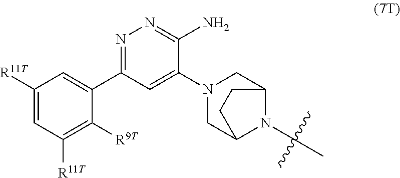

wherein the wavy line intersects the bond between the SMARCA2 binder and the linker; and
wherein:
R^{9T} is hydroxy or amino;
each R^{11T} is independently selected from H, and halo.

In some embodiments, the SMARCA2 binder is of the formula 8T:

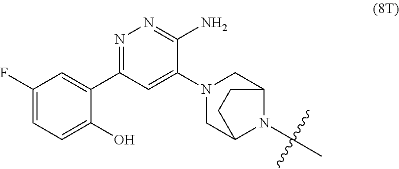 [SMILES: *N1C2CCC1CN(c1cc(-c3cc(F)ccc3O)nnc1N)C2]

wherein the wavy line intersects the bond between the SMARCA2 binder and the linker.

In some embodiments, the SMARCA2 binder is of the formula 9T:

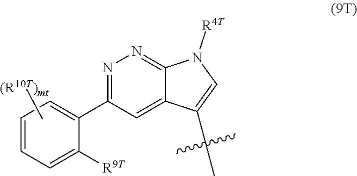

wherein the wavy line intersects the bond between the SMARCA2 binder and the linker; and
wherein:
m is 0, 1, 2, 3, or 4;
R⁴ is selected from hydrogen and C₁-C₄ alkyl;
R⁹ is selected from hydroxy, C₁-C₄ alkoxy, and amino; and
each R¹⁰ is independently selected from hydroxy, C₁-C₄ alkoxy, halo, C₁-C₄ alkyl, and amino.

In some embodiments of the SMARCA binder of formula 9T,
mt is 0, 1 or 2;
R^{4T} is hydrogen;
R^{9T} is selected from hydroxy, C₁-C₄ alkoxy, and amino; and
each R^{10T} is independently selected from hydroxy, C₁-C₄ alkoxy, halo and C₁-C₄ alkyl.

In some embodiments, the SMARCA2 binder is of the formula 10T:

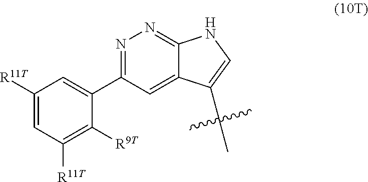

wherein the wavy line intersects the bond between the SMARCA2 binder and the linker; and
wherein:
R^{9T} is hydroxy or amino; and
each R^{11T} is independently selected from H, and halo.

In some embodiments, the SMARCA2 binder is of the formula 11T:

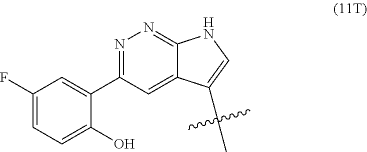 [SMILES: *c1c[nH]c2nnc(-c3cc(F)ccc3O)cc12]

wherein the wavy line intersects the bond between the SMARCA2 binder and the linker.
In some embodiments, the SMARCA2 binder is of the formula 12T:

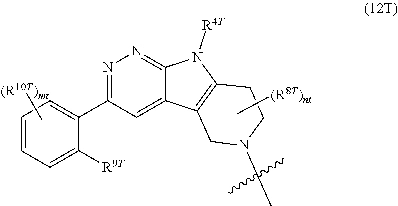

wherein the wavy line intersects the bond between the SMARCA2 binder and the linker; and
wherein:
m is 0, 1, 2, 3, or 4;
n is 0, 1, 2, or 3;
each R^{4T}, R^{7T} and R^{8T} is independently selected from hydrogen and C₁-C₄ alkyl;
R^{9T} is selected from hydroxy, C₁-C₄ alkoxy, and amino; and
each R^{10T} is independently selected from hydroxy, C₁-C₄ alkoxy, halo, C₁-C₄ alkyl, and amino.

In some embodiments of the SMARCA binder of formula 12T,
mt is 0, 1 or 2;
nt is 0;
R^{4T} is hydrogen;
R^{9T} is selected from hydroxy, C₁-C₄ alkoxy, and amino; and
each R^{10T} is independently selected from hydroxy, C₁-C₄ alkoxy, halo and C₁-C₄ alkyl.

In some embodiments, the SMARCA2 binder is of the formula 13T:

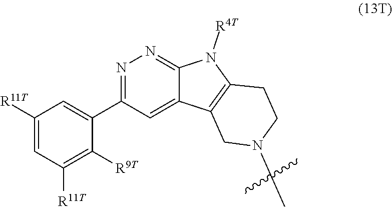

wherein the wavy line intersects the bond between the SMARCA2 binder and the linker; and
wherein:
R⁹ is hydroxy or amino; and
each R¹¹ is independently selected from H, and halo.

In some embodiments, the SMARCA2 binder is of the formula 14T:

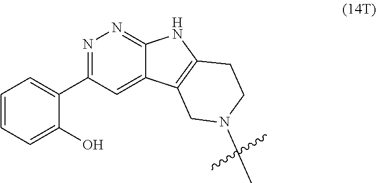 [SMILES: *N1CCc2[nH]c3nnc(-c4ccccc4O)cc3c2C1]

wherein the wavy line intersects the bond between the SMARCA2 binder and the linker.

### I. Kinase and Phosphatase Inhibitors

Examples of kinase inhibitors may be found in Jones et al. Small-Molecule Kinase Downregulators (2017, Cell Chem. Biol., 25:30-35). Further kinase inhibitors that may be used according to some examples of the disclosure include, but are not limited to:

### 1. Erlotinib Derivative Tyrosine Kinase Inhibitor:

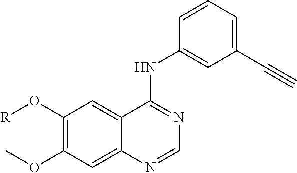

where R is a linker attached, for example, via an ether group;
2. The kinase inhibitor sunitinib (derivatized):

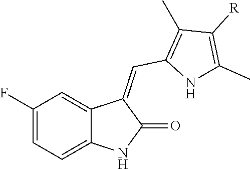

(derivatized where R is a linker attached, for example, to the pyrrole moiety);
3. Kinase Inhibitor sorafenib (derivatized):

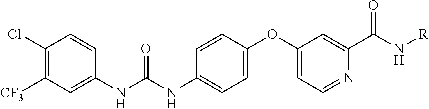

(derivatized where R is a linker attached, for example, to the amide moiety);
4. The kinase inhibitor dasatinib (derivatized):

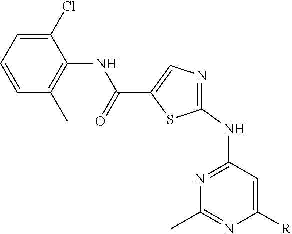

(derivatized where R is a linker attached, for example, to the pyrimidine);
5. The kinase inhibitor lapatinib (derivatized):

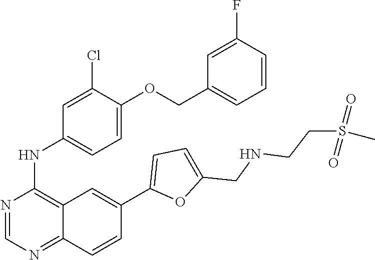 [SMILES: CS(=O)(=O)CCNCc1ccc(-c2ccc3ncnc(Nc4ccc(OCc5cccc(F)c5)c(Cl)c4)c3c2)o1]

(derivatized where a linker is attached, for example, via the terminal methyl of the sulfonyl methyl group);
6. The kinase inhibitor U09-CX-5279 (derivatized):

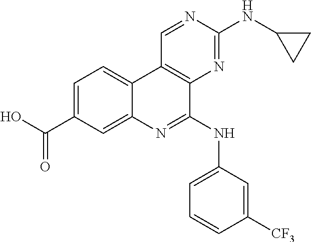 [SMILES: O=C(O)c1ccc2c(c1)nc(Nc1cccc(C(F)(F)F)c1)c1nc(NC3CC3)ncc12]

derivatized where a linker is attached, for example, via the amine (aniline), carboxylic acid or amine alpha to cyclopropyl group, or cyclopropyl group;
7. The kinase inhibitors identified in Millan, et al., Design and Synthesis of Inhaled P38 Inhibitors for the Treatment of Chronic Obstructive Pulmonary Disease, (2011,]. Med. Chem. 54:7797), including the kinase inhibitors Y1W and Y1X (Derivatized) having the structures:

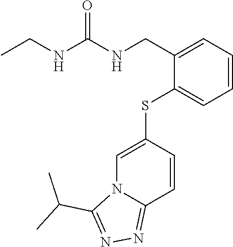 [SMILES: CCNC(=O)NCc1ccccc1Sc1ccc2nnc(C(C)C)n2c1]

### YIX

(I-ethyl-3-(2-{[3-(1-methylethyl)[1,2,4]triazolo[4,3-a]pyridine-6-yl]sulfanyl}benzyl)urea derivatized where a linker is attached, for example, via the iso-propyl group;

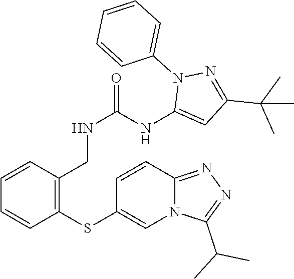 [SMILES: CC(C)c1nnc2ccc(Sc3ccccc3CNC(=O)Nc3cc(C(C)(C)C)nn3-c3ccccc3)cn12]

### Y1W

1-(3-tert-butyl-1-phenyl-1H-pyrazol-5-yl)-3-(2-{[3-(1-methylethyl)[1,2,4]triazolo[4,3-a]pyridin-6-yl]sulfanyl}benzyl)urea derivatized where a linker is attached, for example, preferably via either the iso-propyl group or the tert-butyl group;

8. The kinase inhibitors identified in Schenkel, et al., Discovery of Potent and Highly Selective Thienopyridine Janus Kinase 2 Inhibitors (2011, J. Med. Chem., 54(24):8440-8450), including the compounds 6TP and OTP (Derivatized) having the structures:

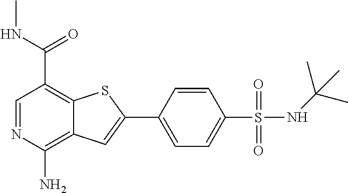 [SMILES: CNC(=O)c1cnc(N)c2cc(-c3ccc(S(=O)(=O)NC(C)(C)C)cc3)sc12]

### 6TP

4-amino-2-[4-(tert-butylsulfamoyl)phenyl]-N-methylthieno[3,2-c]pyridine-7-carboxamide

### Thienopyridine 19

derivatized where a linker is attached, for example, via the terminal methyl group bound to amide moiety;

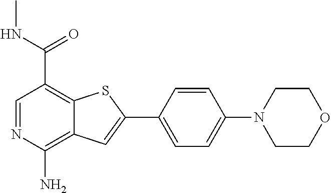 [SMILES: CNC(=O)c1cnc(N)c2cc(-c3ccc(N4CCOCC4)cc3)sc12]

### OTP

4-amino-N-methyl-2-[4-(morpholin-4-yl)phenyl]thieno[3,2-c]pyridine-7-carboxamide

### Thienopyridine 8

derivatized where a linker is attached, for example, via the terminal methyl group bound to the amide moiety;
9. The kinase inhibitors identified in Van Eis, et al., “2,6-Naphthyridines as potent and selective inhibitors of the novel protein kinase C isozymes”, (2011 December, Biorg. Med. Chem. Lett., 15,21(24):7367-72), including the kinase inhibitor 07U having the structure:

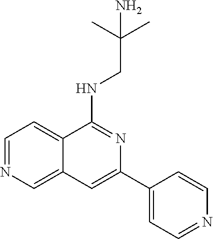 [SMILES: CC(C)(N)CNc1nc(-c2ccncc2)cc2cnccc12]

### 07U

2-methyl-N-1-[3-(pyridin-4-yl)-2,6-naphthyridin-1-yl]propane-1,2-diamine derivatized where a linker is attached, for example, via the secondary amine or terminal amino group;

10. The kinase inhibitors identified in Lountos, et al., “Structural Characterization of Inhibitor Complexes with Checkpoint Kinase 2 (Chk2), a Drug Target for Cancer Therapy”, (2011, J. Struct. Biol., 176:292), including the kinase inhibitor YCF having the structure:

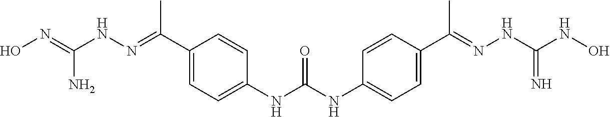 [SMILES: C/C(=N\NC(=N)NO)c1ccc(NC(=O)Nc2ccc(/C(C)=N/N/C(N)=N/O)cc2)cc1]

derivatized where a linker is attached, for example, via either of the terminal hydroxyl groups;

11. The kinase inhibitors identified in Lountos, et al., “Structural Characterization of Inhibitor Complexes with Checkpoint Kinase 2 (Chk2), a Drug Target for Cancer Therapy”, (2011, J. Struct. Biol. 176292), including the kinase inhibitors XK9 and NXP (derivatized) having the structures:

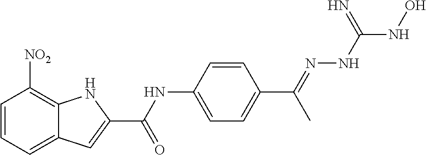 [SMILES: C/C(=N\NC(=N)NO)c1ccc(NC(=O)c2cc3cccc([N+](=O)[O-])c3[nH]2)cc1]

### XK9

N-{4-[(1E)-N—(N-hydroxycarbamimidoyl)ethanehydrazonoyl]phenyl}-7-nitro-1 H-indole-2-carboxamide;

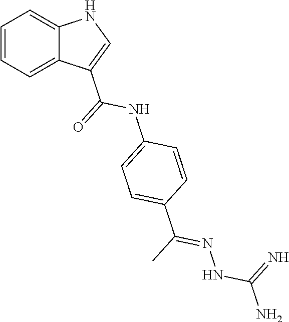 [SMILES: C/C(=N\NC(=N)N)c1ccc(NC(=O)c2c[nH]c3ccccc23)cc1]

### NXP

N-{4-[(1E)-N-carbamimidoylethanehydrazonoyl]phenyl}-1H-indole-3-carboxamide derivatized where a linker is attached, for example, via the terminal hydroxyl group (XK9) or the hydrazone group (NXP);

12. The kinase inhibitor afatinib (derivatized) (N-[4-[(3-chloro-4-fiuorophenyl)amino]-7-[[(3S)-tetrahydro-3-furanyl]oxy]-6-quinazolinyl]-4 (dimethylamino)-2-butenamide) (Derivatized where a linker is attached, for example, via the aliphatic amine group);
13. The kinase inhibitor fostamatinib (derivatized) ([6-({5-fluoro-2-[(3,4,5-trimethoxyphenyl)amino]pyrimidin-4-yl}amino)-2,2-dimethyl-3-oxo-2,3-dihydro-4H-pyrido[3,2-b]-1,4-oxazin-4-yl]methyl disodium phosphate hexahydrate) (Derivatized where a linker is attached, for example, via a methoxy group);
14. The kinase inhibitor gefitinib (derivatized) (N-(3-chloro-4-fluoro-phenyl)-7-methoxy-6-(3-morpholin-4-ylpropoxy)quinazolin-4-amine):

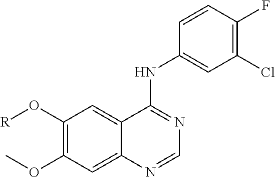

(derivatized where a linker is attached, for example, via a methoxy or ether group);
15. The kinase inhibitor lenvatinib (derivatized) (4-[3-chloro-4-(cyclopropylcarbamoylamino)phenoxy]-7-methoxy-quinoline-6-carboxamide) (derivatized where a linker is attached, for example, via the cyclopropyl group);
16. The kinase inhibitor vandetanib (derivatized) (N-(4-bromo-2-fluorophenyl)-6-methoxy-7-[(I-methylpiperidin-4-yl)methoxy]quinazolin-4-amine) (derivatized where a linker is attached, for example, via the methoxy or hydroxyl group);
17. The kinase inhibitor Gleevec (also known as Imatinib) (derivatized):

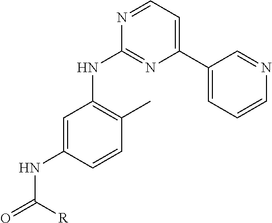

(derivatized where R is a linker attached, for example, via the amide group or via the aniline amine group);
18. The kinase inhibitor pazopanib (derivatized) (VEGFR3 inhibitor):

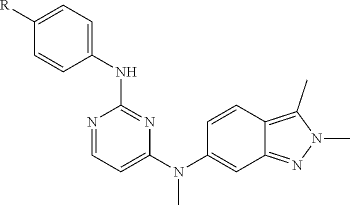

(derivatized where R is a linker attached, for example, to the phenyl moiety or via the aniline amine group);
19. The kinase inhibitor AT-9283 (Derivatized) Aurora Kinase Inhibitor

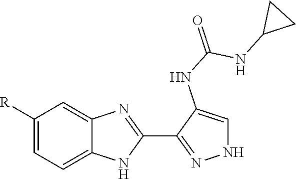

(where R is a linker attached, for example, to the phenyl moiety);
20. The kinase inhibitor TAE684 (derivatized) ALK inhibitor

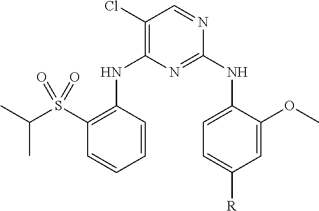

(where R is a linker attached, for example, to the phenyl moiety);
21. The kinase inhibitor nilotinib (derivatized) Abl inhibitor:

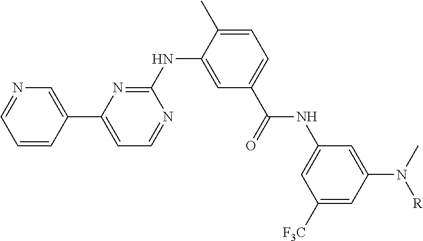

(derivatized where R is a linker attached, for example, to the phenyl moiety or the aniline amine group);
22. Kinase Inhibitor NVP-BSK805 (derivatized) JAK2 Inhibitor

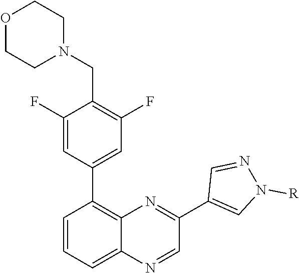

(derivatized where R is a linker attached, for example, to the phenyl moiety or the diazole group);
23. Kinase Inhibitor crizotinib Derivatized Alk Inhibitor

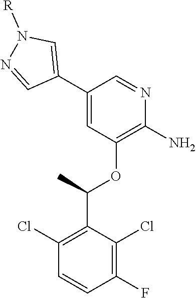

(derivatized where R is a linker attached, for example, to the phenyl moiety or the diazole group);
24. Kinase Inhibitor JNJ FMS (derivatized) Inhibitor

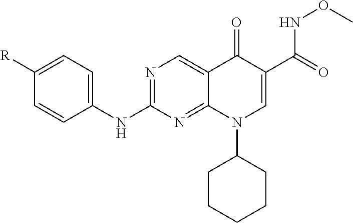

(derivatized where R is a linker attached, for example, to the phenyl moiety);
25. The kinase inhibitor foretinib (derivatized) Met Inhibitor

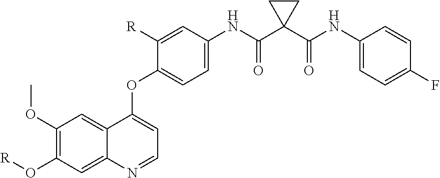

(derivatized where R is a linker attached, for example, to the phenyl moiety or a hydroxyl or ether group on the quinoline moiety);
26. The allosteric Protein Tyrosine Phosphatase Inhibitor PTPIB (derivatized):

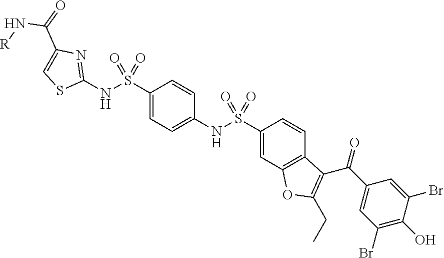

derivatized where a linker is attached, for example, at R, as indicated;
27. The inhibitor of SHP-2 Domain of Tyrosine Phosphatase (derivatized):

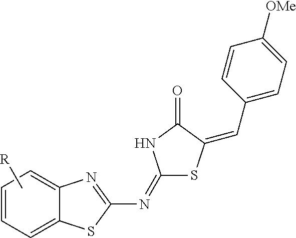

derivatized where a linker is attached, for example, at R;
28. The inhibitors (derivatized) of BRAF (wt and/or mutant forms):

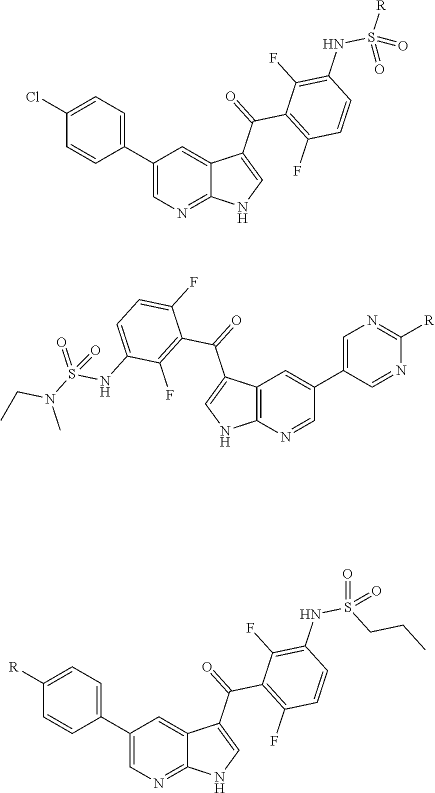

derivatized where a linker group is attached, for example, at R, e.g. the kinase inhibitor vemurafenib (PLX4032) (derivatized) (propane-1-sulfonic acid

{3-[5-(4-chlorophenyl)-1H-pyrrolo[2,3-b]pyridine-3-carbonyl]-2,4-difiuoro-phenyl}-amide)

(derivatized where a linker is attached, for example, via the sulfonyl propyl group);
29. Inhibitor (derivatized) of Tyrosine Kinase ABL

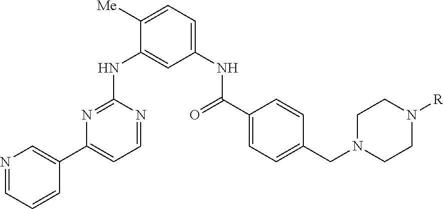

derivatized where a linker is attached, for example, at R;
30. The kinase inhibitor OSI-027 (derivatized) mTORCI/2 inhibitor

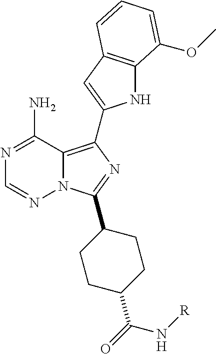

derivatized where a linker is attached, for example, at R;
31. The kinase inhibitor OSI-930 (derivatized) c-Kit/KDR inhibitor

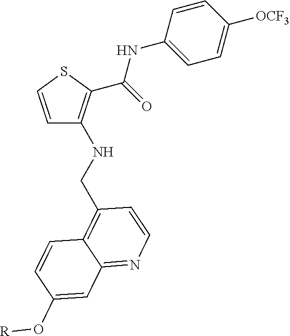

derivatized where a linker is attached, for example, at R; and
32. The kinase inhibitor OSI-906 (derivatized) IGFIR/IR inhibitor

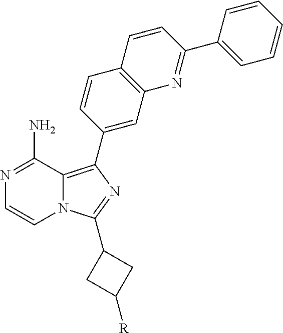

derivatized where a linker is attached, for example, at R;
(derivatized where “R” designates a site for attachment of a linker on the piperazine moiety).

### II. Compounds Targeting Human BET Bromodomain-Containing Proteins

Compounds targeting Human BET Bromodomain-containing proteins include, but are not limited to the compounds associated with the targets as described below, where “R” designates a site for linker attachment, for example:

JQI, Filippakopoulos et al. Selective inhibition of BET bromodomains. Nature (2010):

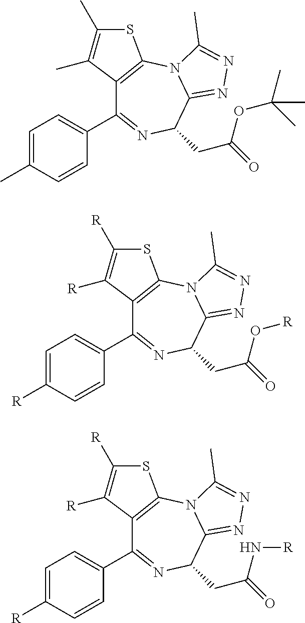

2. I-BET, Nicodeme et al. Supression of Inflammation by a Synthetic Histone Mimic. Nature (2010). Chung et al. Discovery and Characterization of Small Molecule Inhibitors of the BET Family Bromodomains. J. Med Chem. (2011):

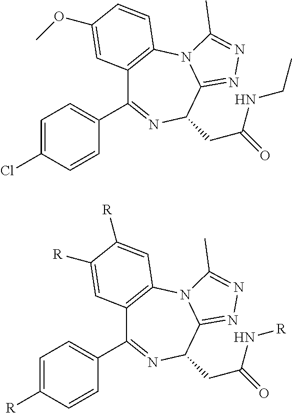

3. Compounds described in Hewings et al. 3,5-Dimethylisoxazoles Act as Acetyl-lysine Bromodomain Ligands. (2011, J. Med. Chem.54:6761-6770).

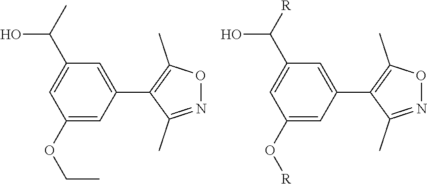

4. I-BET151, Dawson et al. Inhibition of BET Recruitment to Chromatin as an Effective Treatment for MLL-fusion Leukemia. Nature (2011):

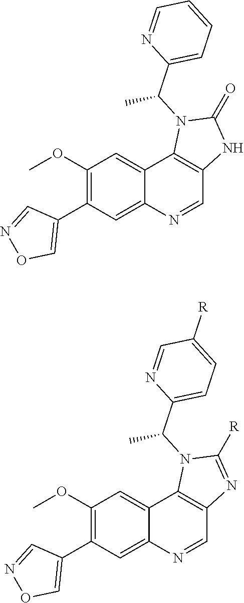

(Where R, in each instance, designates a site for attachment of a linker.)

### BRD9

Representative examples of BRD9 targeting agents have been developed over the years, including those described in: WO 2014/114721, WO 2016/077375, WO 2016/077378, WO 2016/139361, WO 2019/152440, a paper by Martin L. J. et. al., (Journal of Medicinal Chemistry 2016, 59, 4462-4475) titled “Structure-Based Design of an in Vivo Active Selective BRD9 Inhibitor”; a paper by Theodoulou N. H. et. al., (Journal of Medicinal Chemistry 2015, 59, 1425-1439) titled “Discovery of I-BRD9, a selective Cell Active Chemical Probe for Bromodomain Containing Protein 9 Inhibition”; and a paper by Clack P. et. al., (Angewandte Chemie, 2015, 127, 6315-6319).

Such BRD9 binding molecules (as referenced in the paragraph above) can be incorporated into the bifunctional molecules of the present disclosure as the target protein binding ligand (TBL).

The BRD9 binder may be of formula BRD91a:

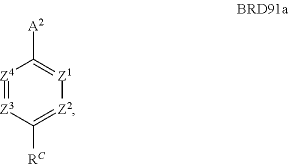

wherein:
Z¹ is N or CR^{A};
Z² is N or CR^{B};
Z³ is N or CR^{D};
Z⁴ is N or CR^{E};
wherein no more than 3 of Z¹, Z², Z³ and Z⁴ are N;
R^{A} and R^{E} are each independently selected from the group consisting of —H, —O—C₁₋₃alkyl and —C₁₋₃alkyl;
R^{B} and R^{D} are each independently selected from the group consisting of —O—C₁₋₃alkyl, —H, —OH, halogen, —NH₂, —C₁₋₃alkyl, —O—C₁₋₃haloalkyl, —C₁₋₃alkyl-O—C₁₋₃alkyl, 4-7 membered heterocycloalkyl, —C₁₋₃alkyl-SO₂-C₁₋₃alkyl, —C₁₋₃alkyl-NH₂, —C₁₋₃alkyl-N(—C₁₋₃alkyl) 2, —N(C₁₋₃alkyl) 2, —NH—R^{F};
R^{F} is selected from —SO₂-C₁₋₃alkyl and —C₁₋₃alkyl, wherein the —C₁₋₃alkyl is optionally substituted with a 5 to 6 membered heteroaryl;
alternatively, R^{A} and R^{B} taken together form a benzene ring;
alternatively, R^{C} and Z² or R^{C} and Z³ taken together (e.g. R^{C} and R^{B} or R^{C} and R^{D} taken together with the carbon atoms to which they are joined) form a 5-7 membered heterocycloalkyl optionally substituted with —C₁₋₃alkyl;
R^{C} is selected from the group consisting of —H, —Y—R^{G}, —NH₂, —C₁₋₃alkyl and 4-7 membered heterocycloalkyl;
Y is absent or is selected from the group consisting of —CR^{H}R^{I}—, —SO₂— and —CO—;
R^{H} and R^{I} are each independently selected from —H or —C₁₋₃alkyl; or R^{H} and R^{I} taken together form a —C₃₋₄cycloalkyl,
R^{G} is selected from the group consisting of —NH₂, —OH, —C₁₋₃alkyl, —N(R^{J}R^{K}), —O—R^{L}, aryl, 5-6 membered heteroaryl, wherein the aryl and heteroaryl are optionally and independently substituted with one or more halogen, optionally substituted 4- to 7-membered monocyclic heterocycloalkyl, and optionally substituted 7- to 12-membered bicyclic heterocycloalkyl, which monocylic or bicyclic heterocycloalkyl are optionally substituted with any suitable substituent, such as one or more groups independently selected from halogen, —OH, —NH₂, —C₁₋₃alkyl, —NHC₁₋₃alkyl, —N(C₁₋₃alkyl)₂, —O—C₁₋₃alkyl and —CH₂-R^{M1};
R^{M1} is selected from 5-10 membered mono- or bicyclic aryl or heteroaryl, which is optionally substituted with —NH₂, —OH, halogen, —CN, C₁₋₃alkyl, —O—C₁₋₃alkyl;
R^{J} is —H or —C₁₋₃alkyl;
R^{K} is selected from the group consisting of —C₁₋₃alkyl, —C₂₋₃alkyl-N(C₁₋₃alkyl)₂, —C₂₋₃alkyl-NHC₁₋₃alkyl, optionally substituted 4- to 7-membered monocyclic heterocycloalkyl, and optionally substituted 7- to 12-membered bicyclic heterocycloalkyl, which monocyclic or bicyclic heterocycloalkyl are optionally substituted with any suitable substituent, such as —C₁₋₃alkyl;
R^{L} is —C₁₋₃alkyl or a 4-7 membered heterocycloalkyl, which heterocycloalkyl is optionally substituted with C₁₋₃alkyl;
wherein when R^{C} is Y—R^{G}, R^{B} and R^{D} are each independently selected from —H, —OH, halogen, —NH₂, —CN, —C₁₋₃alkyl, —C₁₋₃haloalkyl, —O—C₁₋₃alkyl, —O—C₁₋₃haloalkyl and —C₁₋₃alkyl-O—C₁₋₃alkyl;
wherein at least one of the substituents R^{A} to R^{E} is not hydrogen;
and
A² is selected from formulae 1b or 1c:

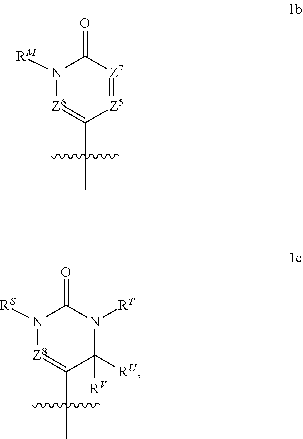

wherein the wavy lines intersect the bond between A² and the carbon atom positioned ortho to R^{A} and R^{E};
R^{M} is selected from the group consisting of optionally substituted C₁₋₆alkyl, optionally substituted C₂₋₆alkenyl, optionally substituted C₁₋₆heteroalkyl, optionally substituted C₃₋₁₀carbocyclyl, C₂₋₆alkynyl and H;
Z⁵ is N or CR^{O};
Z⁶ is N or CR^{P};
Z⁷ is N or CR^{N};
wherein only one of Z⁵, Z⁶ and Z⁷ is N;
Z⁸ is CR^{W} or N;
R^{N} is selected from the group consisting of halogen, optionally substituted —C₁₋₆alkyl, —H, C(O)C₁₋₅alkyl, —NH₂, optionally substituted amino, —OH, cyano, optionally substituted C₁₋₆heteroalkyl, optionally substituted C₃₋₁₀ carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl and thiol;
R^{O} is selected from the group consisting of H, halogen, cyano, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, hydroxy, thiol and optionally substituted amino;
R^{P} is selected from the group consisting of H, halogen, optionally substituted C₁₋₆alkyl, optionally substituted C₁₋₆heteroalkyl, optionally substituted C₃₋₁₀carbocyclyl and optionally substituted C₆₋₁₀aryl;
alternatively, R^{N} and Z⁵ taken together, combine to form an optionally substituted C₆₋₁₀arene or optionally substituted C₂₋₉heteroarene; optionally wherein R^{N} and R^{O}taken together with the carbon atoms to which they are joined, combine to form an optionally substituted C₆₋₁₀arene or optionally substituted C₂₋₉heteroarene;
R^{S} is selected from the group consisting of H, optionally substituted C₁₋₆alkyl, optionally substituted C₁₋₆heteroalkyl and optionally substituted C₃₋₁₀carbocyclyl;
R^{T} is selected from the group consisting of H, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, optionally substituted sulfone and optionally substituted sulfonamide, or R^{T} and R^{U} together with the atoms to which each is attached, form an optionally substituted C₂₋₉heterocyclyl;
R^{U} and R^{V} are each independently selected from the group consisting of H, halogen, hydroxyl, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, thiol, optionally substituted sulfone and optionally substituted amino; alternatively, R^{T} and R^{U} together with the atoms to which each is attached, form an optionally substituted C₂₋₉heterocyclyl;
R^{W} is selected from the group consisting of H, halogen, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl and optionally substituted C₂₋₉heteroaryl;
and
wherein the BRD9 binder is attached to the linker at any suitable position

In some embodiments, no more than 1 of Z¹, Z², Z³ and Z⁴ of formula BRD91a is N. Sometimes, Z¹ is CR^{A}, Z² is CR^{B}, Z³ is N or CR^{D} and Z⁴ is CR^{E}, i.e. only Z³ may be N. In such embodiments, the BRD9 binder may be of formula BRD91a′:

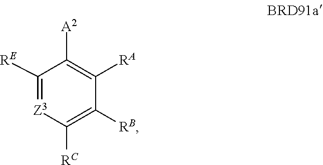

wherein:
R^{A}, R^{B}, R^{C}, R^{E}, Z³ and A² are as defined above and herein.
A² is selected from formulae BRD91b or BRD91c:

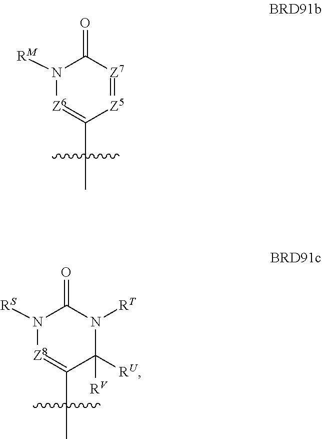

wherein the wavy lines intersect the bond between A² and the carbon atom positioned ortho to R^{A} and R^{E}, and Z⁵, Z⁶, Z⁷, Z⁸, R^{M}, R^{S}, R^{T}, R^{U} and R^{V} are as defined above and herein.

Z⁷ is N or CR^{N} and Z⁵ is N or CR^{O}. In some examples, R^{N} (with the carbon to which it is bonded) and Z⁵ taken together, may combine to form an optionally substituted C₆₋₁₀arene or optionally substituted C₂₋₉heteroarene. For the avoidance of doubt, where Z⁵ is N and RN (with the carbon to which it is bonded) and Z⁵ taken together combine to form an optionally substituted C₆₋₁₀arene or optionally substituted C₂₋₉heteroarene, R^{N} (with the carbon to which it is bonded) and Z⁵ taken together combine to form an optionally substituted N—C₂₋₄heteroarene. For example, where Z⁵ is N, R^{N} and N may combine to form an optionally substituted N—C₂₋₄heteroaryl, as shown below:

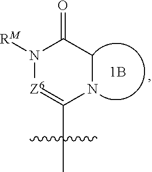

wherein the wavy lines intersect the bond between A² and the carbon atom positioned ortho to R^{A} and R^{E}, Z⁶ and R^{M} are as defined above, and where 1B is an optionally substituted N—C₂₋₄heteroarene, such as an optionally substituted 5 membered heteroarene e.g. any one selected from the optionally substituted group consisting of pyrrole, imidazole, pyrazole and triazole (including 1,2,3 and 1,2,4-triazoles).

In some examples, where Z⁵ is CR^{O} and Z⁷ is CR^{N}, R^{N} and R^{O}taken together with the carbons to which they are bonded, may combine to form an optionally substituted C₆₋₁₀arene or optionally substituted C₂₋₉heteroarene, as shown below:

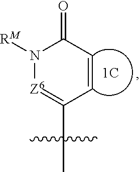

wherein the wavy lines intersect the bond between A² and the carbon atom positioned ortho to R^{A} and R^{E}, Z⁶ and R^{M} are as defined above, and where, as stated above, ring 1C is an optionally substituted C₆₋₁₀arene or optionally substituted C₂₋₉heteroarene. For example, ring 1C may be an optionally substituted benzene or 5-6 membered heteroarene, such as any one selected from the optionally substituted group consisting of benzene, pyridine, pyrrole, imidazole, pyrimidine, thiophene and pyrazole.

In some embodiments, R^{N} (taken with the carbon atoms to which it is joined) and Z⁵ taken together may form a benzene ring or a 5-6 membered heteroarene ring (e.g. ring 1C may be a benzene ring or a 5-6 membered heteroarene), each of which rings can be optionally and independently substituted with one or more groups selected from halogen, —OH, —NH₂, —NH—C₁₋₃alkyl and —C₁₋₅alkyl, C₁₋₅haloalkyl, C₁₋₅alkoxy, C₁₋₄haloalkoxy, 1d, C₃₋₅azacycloalkyl, C₂₋₅alkenyl, C₂₋₅alkynyl, C₃₋₅cycloalkyl, wherein the —C₁₋₅alkyl group can be optionally substituted with 5-6 membered heteroaryl or phenyl; wherein 1d is:

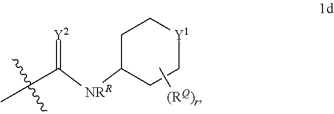

wherein
Y² is NR^{R} or O;
Y¹ is S(O)ₐ or NR^{R};
each R^{R} is independently H or C₁₋₄alkyl;
each R^{Q} is independently selected from the group consisting of C₁₋₄alkyl, C₁₋₄haloalkyl, halogen and —C(O)C₁₋₃alkyl;
a is 0 to 2; and
r is 0 to 3.

In some embodiments, Z⁷ is CR^{N}, i.e. A² is selected from formula BRD91b′:

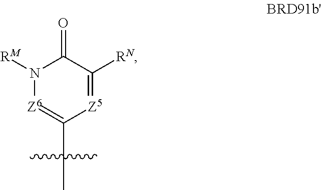

wherein the wavy line intersects the bond between A² and the carbon atom positioned ortho to R^{A} and R^{E}, and Z⁵, Z⁶, RM and R^{N} are as defined above and herein.

R^{M} may be selected from the group consisting of optionally substituted C₁₋₆alkyl, optionally substituted C₂₋₆alkenyl, optionally substituted C₁₋₆heteroalkyl, optionally substituted C₃₋₁₀carbocyclyl, C₂₋₆alkynyl and H. In some embodiments, R^{M} may be selected from the group consisting of optionally substituted C₁₋₆alkyl, optionally substituted C₃₋₆cycloalkyl and H. For example, R^{M} may be selected from the group consisting of C₁₋₆alkyl, C₃₋₆cycloalkyl, C₁₋₆haloalkyl and H. In some embodiments, R^{M} is selected from the group consisting of —C₁₋₅alkyl, -cyclopropyl, —C₁₋₄haloalkyl and H, such as C₁₋₅alkyl. In some embodiments, R^{M} is C₁₋₃alkyl.

R^{N} may be selected from the group consisting of halogen, optionally substituted —C₁₋₆alkyl, —H, C(O)C₁₋₅alkyl, —NH₂, optionally substituted amino, —OH, cyano, optionally substituted C₁₋₆heteroalkyl, optionally substituted C₃₋₁₀ carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl and thiol. In some embodiments, R^{N} may be selected from the group consisting of halogen, optionally substituted C₁₋₆alkyl, H, C(O)C₁₋₅alkyl, —NH₂, —NHC₁₋₃alkyl and —OH. In some embodiments, R^{N} is selected from the group consisting of halogen, —C₁₋₅alkyl, —C₁₋₃haloalkyl, —H, C(O)C₁₋₅alkyl, —NH₂, —NHC₁₋₃alkyl and —OH. For example, R^{N} may be C₁₋₅alkyl or halogen.

Z⁵ may be N or CR^{O}, where R^{O} is selected from the group consisting of H, halogen, cyano, optionally substituted C₁₋₆alkyl, optionally substituted C₁₋₆heteroalkyl, optionally substituted C₃₋₁₀carbocyclyl, optionally substituted C₂₋₉heterocyclyl, optionally substituted C₆₋₁₀aryl, optionally substituted C₂₋₉heteroaryl, optionally substituted C₂₋₆alkenyl, optionally substituted C₂₋₆heteroalkenyl, hydroxy, thiol and optionally substituted amino. For example, R^{O} may be H or optionally substituted C₁₋₆alkyl, such as C₁₋₃alkyl. In some embodiments, R^{O} may be H or —C₁₋₃alkyl.

In some embodiments, R^{N} is —C₁₋₅alkyl or halogen, or R^{N} and Z⁵ taken together form an optionally substituted 5-6 membered heteroarene or benzene ring. In some embodiments, the optionally substituted 5-6 membered heteroarene ring may comprise one or more heteroatoms selected from the group consisting of N, S and O, such as N and S, i.e. the optionally substituted 5-6 membered heteroarene ring may be an N- or S-heteroarene. In some embodiments, the optionally substituted 5-6 membered heteroarene ring is any one selected from the optionally substituted group consisting of pyridine, pyrrole, imidazole, pyrimidine, thiophene and pyrazole.

For the avoidance of doubt, the optional substituents may be one or more groups selected from halogen, —OH, —NH₂, —NH—C₁₋₃alkyl and —C₁₋₅alkyl, C₁₋₅haloalkyl, C₁₋₅alkoxy, C₁₋₄haloalkoxy, 1d, C₃₋₅azacycloalkyl, C₂₋₅alkenyl, C₂₋₅alkynyl, C₃₋₅cycloalkyl, wherein the —C₁₋₅alkyl group can be optionally substituted with 5-6 membered heteroaryl or phenyl; wherein 1d is:

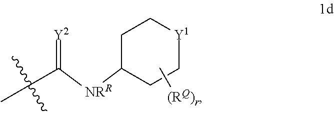

wherein
Y² is NR^{R} or O;
Y¹ is S(O)ₐ or NR^{R};
each R^{R} is independently H or C₁₋₄alkyl;
each R^{Q} is independently selected from the group consisting of C₁₋₄alkyl, C₁₋₄haloalkyl, halogen and —C(O)C₁₋₃alkyl;
a is 0 to 2; and
r is 0 to 3.

For example, the optional substituents may be independently selected from the group consisting of halogen, —OH, —NH₂, —NH—C₁₋₃alkyl-C₁₋₅alkyl, C₁₋₅haloalkyl, C₁₋₅alkoxy and C₁₋₄haloalkoxy. In some cases, the optional substituents may be independently selected from C₁-C₄alkyl, allyl, crotyl, C₂₋₅alkenyl, C₂₋₅alkynyl, C₁₋₅haloalkyl, C₃₋₅cycloalkyl, C₁-C₄alkoxy, and halo. In some embodiments, where R^{N} and Z⁵ taken together combine to form an optionally substituted C₆₋₁₀aryl or optionally substituted C₂₋₉heteroaryl, the C₆₋₁₀aryl or C₂₋₉heteroaryl is not substituted.

Z⁶ may be N or CR^{P}, where R^{P} is selected from the group consisting of H, halogen, optionally substituted C₁₋₆alkyl, optionally substituted C₁₋₆heteroalkyl, optionally substituted C₃₋₁₀carbocyclyl and optionally substituted C₆₋₁₀aryl. For example, RP may be H or optionally substituted C₁₋₆alkyl, such as H or C₁₋₆alkyl. In some embodiments, R^{P} is H or —C₁₋₃alkyl, i.e. Z⁶ is N, CH or C—C₁₋₃alkyl. For example, Z⁶ may be CH or C—C₁₋₃alkyl.

In some particular embodiments, A² is selected from formula BRD91b′, wherein formula BRD91b′ is:

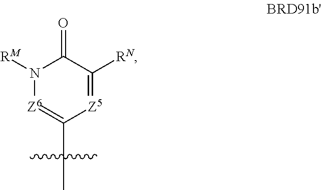

wherein the wavy line intersects the bond between A² and the carbon atom positioned ortho to R^{A} and R^{E};
R^{M} is selected from the group consisting of —C₁₋₅alkyl, -cyclopropyl, —C₁₋₄haloalkyl and H;
R^{N} is selected from the group consisting of halogen, —C₁₋₅alkyl, —C₁₋₃haloalkyl, —H, C(O)C₁₋₅alkyl, —NH₂, —NHC₁₋₃alkyl and —OH;
Z⁵ is N or CR^{O}
Z⁶ is N or CRP wherein only one of Z⁵ and Z⁶ may be N;
R^{O} is H or —C₁₋₃alkyl;
R^{P} is H or —C₁₋₃alkyl;
wherein only one of R^{O} and R^{P} may be —C₁₋₃alkyl;
alternatively, R^{N} and Z⁵ taken together form a benzene ring or a 5-6 membered heteroarene ring, each of which rings can be optionally and independently substituted with one or more groups selected from halogen, —OH, —NH₂, —NH—C₁₋₃alkyl and —C₁₋₅alkyl, C₁₋₅haloalkyl, C₁₋₅alkoxy, C₁₋₄haloalkoxy, 1d, C₃₋₅azacycloalkyl, C₂₋₅alkenyl, C₂₋₅alkynyl, C₃₋₅cycloalkyl, wherein the —C₁₋₅alkyl group can be optionally substituted with 5-6 membered heteroaryl or phenyl;
wherein 1d is:

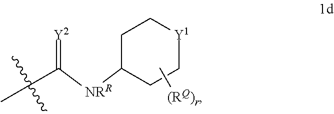

wherein
Y² is NR^{R} or O;
Y¹ is S(O)ₐ or NR^{R};
each R^{R} is independently H or C₁₋₄alkyl;
each R^{Q} is independently selected from the group consisting of C₁₋₄alkyl, C₁₋₄haloalkyl, halogen and —C(O)C₁₋₃alkyl;
a is 0 to 2; and
r is 0 to 3.

The BRD9 binder may be attached to the linker at any suitable position (provided it has the correct valency and/or is chemically suitable). For example, the linker may be attached to the BRD9 binder by way of a covalent bond between an atom on the linker and an atom forming part of R^{C}, R^{A}, R^{B}, R^{D} or R^{E}. Alternatively, the linker may be attached directly to the ring to which R^{C}, R^{A}, R^{B}, R^{D} and/or R^{E} are bound, i.e. the linker may replace R^{C}, R^{A}, R^{B}, R^{D} or R^{E}. In some embodiments, the linker is attached to the BRD9 binder by way of a covalent bond between an atom on the linker and an atom forming part of R^{C} or by way of a covalent bond between an atom on the linker and the atom to which R^{C} would otherwise be bound, i.e. the linker replaces R^{C}.

Alternatively, where R^{C} and Z² or R^{C} and Z³ taken together (e.g. R^{C} and R^{B} or R^{C} and R^{D} taken together with the carbon atoms to which they are joined) form a 5-7 membered heterocycloalkyl optionally substituted with —C₁₋₃alkyl, the linker may be attached to the BRD9 binder by way of a covalent bond between an atom on the linker and an atom forming part of the 5-7 membered heterocycloalkyl.

In some embodiments, the BRD9 binder is of formula BRD91a¹, BRD91a², BRD91a³:

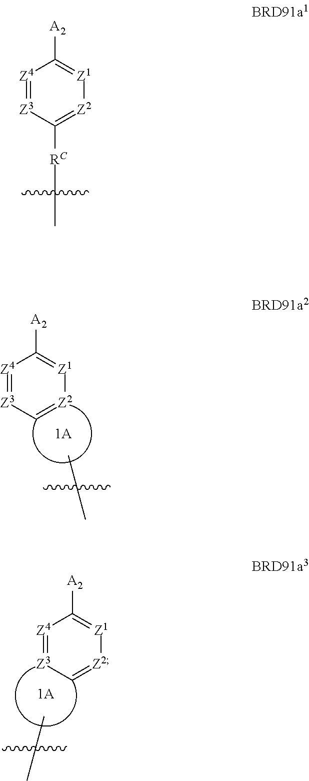

wherein the wavy line intersects the bond between the BRD9 binder and the linker;
A², Z¹, Z², Z³ and Z⁴ are as defined above and herein;
R^{C} is absent or is as defined above and herein;
and ring 1A is a 5-7 membered heterocycloalkane optionally substituted with —C₁₋₃alkyl.

Ring 1A may comprise one or two heteroatoms independently selected from the list consisting of N, S and O. For example, ring 1A may be selected from the list consisting of pyrrolidine, piperidine, piperazine, morpholine, oxolane, oxane, tetrahydrothiophene and thiane. In some cases, ring 1A may be an N-heterocycloalkane such as pyrrolidine, piperidine or piperazine. In particular examples, ring 1A is pyrrolidine.

For the avoidance of doubt, where the linker is attached to the BRD9 binder by way of a covalent bond between an atom on the linker and an atom forming part of a feature on the BRD9 binder (such as R^{C}), the linker replaces a chemical group or an atom of the feature with a valency of 1 (such as a hydrogen atom) in order for valencies to be satisfied. For example, where the feature on the BRD9 binder is dimethylamido (—C(O)N(CH₃)₂) or dimethylaminomethylene (—CH₂N(CH₃)₂), the linker may replace a methyl group or a hydrogen atom on the feature.

As another alternative, the linker may be attached to the BRD9 binder by way of a covalent bond between an atom on the linker and an atom forming part of A², for example an atom forming part of R^{M}, R^{N}, R^{O}, R^{P}, R^{S}, R^{T}, R^{U}, R^{V}, or R^{W} or the linker may replace R^{M}, R^{N}, R^{O}, R^{P}, R^{S}, R^{T}, R^{U}, R^{V}, or R^{W}. Alternatively, where R^{N} and Z⁵ taken together combine to form an optionally substituted C₆₋₁₀arene or optionally substituted C₂₋₉heteroarene; optionally wherein R^{N} and R^{O} taken together with the carbon atoms to which they are joined, combine to form an optionally substituted C₆₋₁₀arene or optionally substituted C₂₋₉heteroarene, the linker may be attached to the BRD9 binder by way of a covalent bond between an atom on the linker and an atom forming part of the optionally substituted C₆₋₁₀arene or optionally substituted C₂₋₉heteroarene.

In one exemplary BRD9 binder, where R^{N} and Z⁵ taken together combine to form an optionally substituted thiophene, the linker may be attached to the BRD9 binder as shown in the structure below:

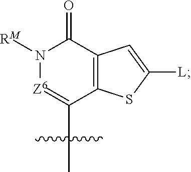

wherein the wavy line intersects the bond between A² and the carbon atom positioned ortho to R^{A} and R^{E}; and R^{M} and Z⁶ are as defined above and herein.

The linker may be attached to an atom forming part of a substituent bonded to the same positions indicated above. For example, the linker may be attached to an atom forming part of substituent 1d bonded to the same positions indicated above. This is exemplified in the structure below, where R^{N} and Z⁵ taken together combine to form an optionally substituted thiophene; the wavy line intersects the bond between A² and the carbon atom positioned ortho to R^{A} and R^{E}; and Y² is O, Y¹ is N, R^{R} is H, and R_{Q} and r are as defined above:

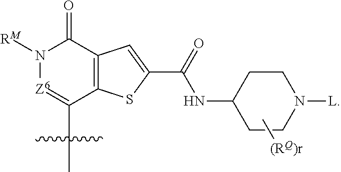

As defined above, Z¹, Z², Z³, Z⁴ and R^{C} of the BRD9 binder may be defined as follows:
Z¹ is N or CR^{A};
Z² is N or CR^{B};
Z³ is N or CR^{D};
Z⁴ is N or CR^{E};
wherein no more than 3 of Z¹, Z², Z³ and Z⁴ are N;
R^{A} and R^{E} are each independently selected from the group consisting of —H, —O—C₁₋₃alkyl and —C₁₋₃alkyl;
R^{B} and R^{D} are each independently selected from the group consisting of —O—C₁₋₃alkyl, —H, —OH, halogen, —NH₂, —C₁₋₃alkyl, —O—C₁₋₃haloalkyl, —C₁₋₃alkyl-O—C₁₋₃alkyl, 4-7 membered heterocycloalkyl, —C₁₋₃alkyl-SO₂-C₁₋₃alkyl, —C₁₋₃alkyl-NH₂, —C₁₋₃alkyl-N(—C₁₋₃alkyl) 2, —N(C₁₋₃alkyl)₂, —NH—R^{F};
R^{F} is selected from —SO₂—C₁₋₃alkyl and —C₁₋₃alkyl, wherein the —C₁₋₃alkyl is optionally substituted with a 5 to 6 membered heteroaryl;
alternatively, R^{A} and R^{B} taken together form a benzene ring; alternatively, R^{C} and Z² or R^{C} and Z³ taken together (e.g. R^{C} and R^{B} or R^{C} and R^{D} taken together with the carbon atoms to which they are joined) form a 5-7 membered heterocycloalkyl optionally substituted with —C₁₋₃alkyl;
R^{C} is selected from the group consisting of —H, —Y—R^{G}, —NH₂, —C₁₋₃alkyl and 4-7 membered heterocycloalkyl;
Y is absent or is selected from the group consisting of —CR^{H}R^{I}—, —SO₂— and —CO—;
R^{H} and R^{I} are each independently selected from —H or —C₁₋₃alkyl; or R^{H} and R^{I} taken together form a —C₃₋₄cycloalkyl,
R^{G} is selected from the group consisting of —NH₂, —OH, —C₁₋₃alkyl, —N(R^{J}R^{K}), —O—R^{L}, aryl, 5-6 membered heteroaryl, wherein the aryl and heteroaryl are optionally and independently substituted with one or more halogen, optionally substituted 4- to 7-membered monocyclic heterocycloalkyl, and optionally substituted 7- to 12-membered bicyclic heterocycloalkyl, which monocyclic or bicyclic heterocycloalkyl are optionally substituted with any suitable substituent, such as one or more groups independently selected from halogen, —OH, —NH₂, —C₁₋₃alkyl, —NHC₁₋₃alkyl, —N(C₁₋₃alkyl) 2, —O—C₁₋₃alkyl and —CH₂—R^{M1};
R^{M1} is selected from 5-10 membered mono- or bicyclic aryl or heteroaryl, which is optionally substituted with —NH₂, —OH, halogen, —CN, C₁₋₃alkyl, —O—C₁₋₃alkyl;
R^{J} is —H or —C₁₋₃alkyl;
R^{K} is selected from the group consisting of —C₁₋₃alkyl, —C₂₋₃alkyl-N(C₁₋₃alkyl) 2, —C₂₋₃alkyl-NHC₁₋₃alkyl, optionally substituted 4- to 7-membered heterocycloalkyl, and optionally substituted 7-to 12-membered bicyclic heterocycloalkyl, which monocyclic or bicyclic heterocycloalkyl are optionally substituted with any suitable substituent, such as —C₁₋₃alkyl;
R^{L} is —C₁₋₃alkyl or a 4-7 membered heterocycloalkyl, which heterocycloalkyl is optionally substituted with C₁₋₃alkyl;
wherein when R^{C} is Y—R^{G}, R^{B} and R^{D} are each independently selected from —H, —OH, halogen, —NH₂, —CN, —C₁₋₃alkyl, —C₁₋₃haloalkyl, —O—C₁₋₃alkyl, —O—C₁₋₃haloalkyl and —C₁₋₃alkyl-O—C₁₋₃alkyl;
wherein at least one of the substituents R^{A} to R^{E} is not hydrogen.

In some examples of the BRD9 binding ligands described herein (and unless otherwise stated):
(i) R^{A}, R^{B}, R^{D} and R^{E} are independently selected from the group consisting of —O—C₁₋₃ alkyl, —H, halogen, —O—C₁₋₃haloalkyl, —OH, —NH₂, —C₁₋₃alkyl, —C₁₋₃alkyl-NH₂, —C₁₋₃alkyl-N(—C₁₋₃alkyl)₂ and —N(C₁₋₃alkyl) 2; or
(ii) R^{A}, R^{D} and R^{E} are independently selected from the group consisting of —O—C₁₋₃alkyl, —H, halogen, —O—C₁₋₃haloalkyl, —OH, —NH₂, —C₁₋₃alkyl, —C₁₋₃alkyl-NH₂, —C₁₋₃alkyl-N(—C₁₋₃alkyl)₂ and —N(C₁₋₃alkyl)₂ and R^{B} and R^{C} taken together form a 5-7 membered heterocycloalkyl optionally substituted with —C₁₋₃alkyl.

In such embodiments, the 5-7 membered heterocycloalkyl may be as defined above for ring 1A.

In some embodiments, R^{A}, R^{B}, R^{D} and R^{E} are independently selected from the group consisting of —O—C₁₋₃alkyl, —H, halogen, —O—C₁₋₃haloalkyl, —OH, —NH₂, —C₁₋₃alkyl, —C₁₋₃alkyl-NH₂, —C₁₋₃alkyl-N(—C₁₋₃alkyl)₂ and —N(C₁₋₃alkyl)₂. For example, R^{A}, R^{B}, R^{D} and R^{E} may be independently selected from the group consisting of —O—C₁₋₃alkyl, —H, halogen and —O—C₁₋₃haloalkyl.

In some cases, at least one of R^{A}, R^{B}, R^{D} and R^{E} may be —H. For example, at least one of R^{A} and R^{B} may be —H. In particular embodiments, at least two of R^{A}, R^{B}, R^{D} and R^{E} are —H.

In some embodiments, at least one of R^{A}, R^{B}, R^{D} and R^{E} is selected from the group consisting of —O—C₁₋₃alkyl, halogen and —O—C₁₋₃haloalkyl. Sometimes, R^{B} and R^{E} are selected from the group consisting of —O—C₁₋₃alkyl, halogen and —O—C₁₋₃haloalkyl.

In some embodiments, R^{C} is —H or —Y—R^{G}. Y may be —CR^{H}R^{I}— or —CO—, wherein R^{H} and R^{I} are as defined above. Each of R^{H} and R^{I} may be —H; or R^{H} and R^{I} taken together may form a —C₃₋₄cycloalkyl.

R^{G} may be as defined above, or may be selected from the group consisting of —NH₂, —OH, —C₁₋₃alkyl-N(R^{J}R^{K}), —O—R^{L} and optionally substituted 4- to 7-membered monocyclic heterocycloalkyl, and optionally substituted 7- to 12-membered bicyclic heterocycloalkyl, where R^{J}, R^{K} and R^{L} and the optional substituents of the 4- to 7-membered monocyclic heterocycloalkyl and 7- to 12-membered bicyclic heterocycloalkyl are as defined above. R^{J} may be —H or —C₁₋₃alkyl and R^{K} may be selected from —C₁₋₃alkyl, optionally substituted 4- to 7-membered monocyclic heterocycloalkyl, and optionally substituted 7- to 12-membered bicyclic heterocycloalkyl. R^{L} may be —C₁₋₃alkyl.

Where R^{G} or R^{K} is an optionally substituted 4- to 7-membered monocyclic heterocycloalkyl, the optionally substituted 4- to 7-membered monocyclic heterocycloalkyl may be a 5- to 7-membered monocyclic heterocycloalkyl comprising between one and three ring heteroatoms selected from N, O and S. In some examples, the optionally substituted 4- to 7-membered monocyclic heterocycloalkyl may be a 5- to 7-membered monocyclic heterocycloalkyl comprising one or two ring heteroatoms selected from N. In some examples, the optionally substituted 4- to 7-membered monocyclic heterocycloalkyl may be piperazinyl, piperidinyl or diazepanyl (each of which may optionally comprise between one and three substituents as described herein).

Where R^{G} or R^{K} is an optionally substituted 7- to 12-membered bicyclic heterocycloalkyl, the optionally substituted 7- to 12-membered bicyclic heterocycloalkyl may be a bridged bicyclic ring or a spirocyclic bicyclic ring (i.e it may comprise two rings joined at a spiro centre). By way of example only, the optionally substituted 7- to 12-membered bicyclic heterocycloalkyl may be a bridged piperazinyl or bridged piperidinyl. In other examples, the optionally substituted 7-to 12-membered bicyclic heterocycloalkyl may be an optionally substituted spirocyclic bicyclic heterocycloalkyl comprising between one and three ring heteroatoms selected from N, O and S (e.g. between one and two ring heteroatoms selected from N). In some examples, the optionally substituted 7- to 12-membered bicyclic heterocycloalkyl may be spirocyclic and comprise a first 5- or 6-membered ring and a second 3- to 6-membered ring.

In some examples, R^{C} may be any one selected from:

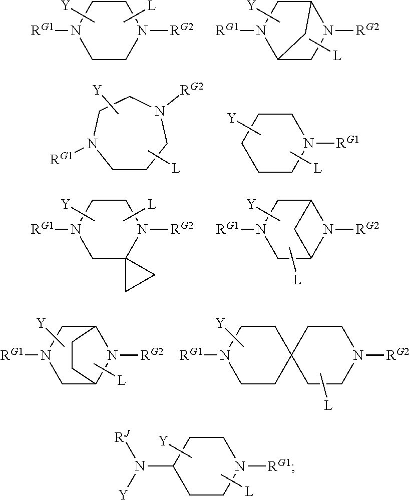

wherein Y is CR^{H}R^{I} (e.g. CH₂);
R^{G1} and R^{G2} are each independently selected from H and C₁-C₃ alkyl;
R^{J} is as defined above and herein; and
L shows the point of attachment of the linker.

In the structures shown above, both the Y and L groups may be attached to the heterocyclic ring(s) by way of a covalent bond between an atom on the Y and L group respectively and an atom on the heterocyclic ring. These groups may be bonded at any chemically suitable position provided valencies are satisfied (e.g. by replacing each replacing a H atom).

By way of further example only, R^{C} may be any one selected from:

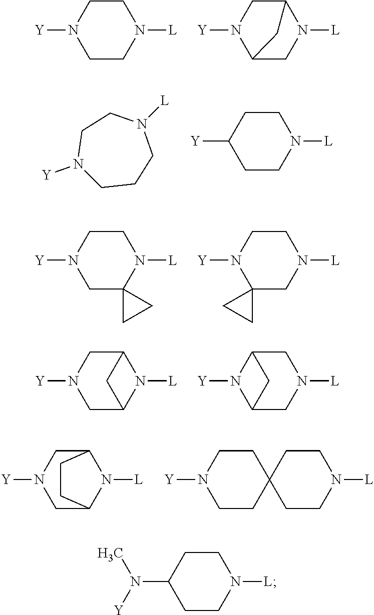

wherein Y is CR^{H}R^{I} (e.g. CH₂); and
L shows the point of attachment of the linker.

In particular embodiments, R^{C} is any one selected from the group consisting of

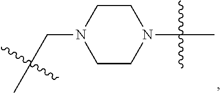 [SMILES: *CN1CCN(*)CC1]

—CH₂N(C₁₋₃alkyl)₂, —C(O)N(C₁₋₃alkyl)₂, —C(CH₂CH₂)N(C₁₋₃alkyl)₂, and CH₂OCH₃, wherein the wavy lines intersect the bond between R^{C} and the rest of the BRD9 binder and the bond between R^{C} and the linker.

In some embodiments, the BRD9 binder is of formula BRD91e, BRD91f or BRD91g:

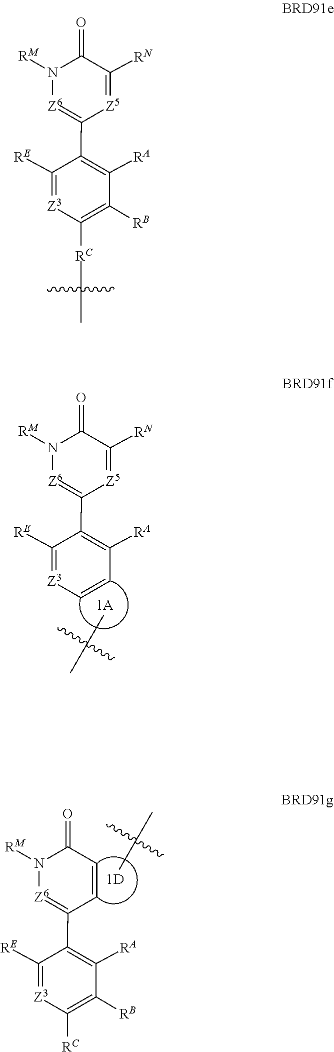

wherein the wavy line intersects the bond between the BRD9 binder and the linker;
R^{A}, R^{B}, R^{E}, R^{M}, R^{N}, Z³, Z⁵ and Z⁶ are as defined above;
R^{C} is absent, or is as defined for R^{C} above and herein;
ring 1A is a 5-7 membered heterocycloalkane optionally substituted with —C₁₋₃alkyl; and
ring 1D is an optionally substituted C₆₋₁₀arene or optionally substituted C₂₋₉heteroarene.

In some embodiments, ring 1D is optionally substituted benzene or an optionally substituted 5-6 membered heteroarene. The 5-6 membered heteroarene may comprise one or more heteroatoms selected from the group consisting of S, N and O, such as S. In some cases, ring 1D may be a 5-6 membered N-heteroarene or S-heteroarene, for example any one selected from the group consisting of thiophene, pyrazole, imidazole, pyrrole, pyrimidine and pyridine. In particular examples, ring 1D is thiophene fused to the rest of the BRD9 binder at the 2′ and 3′ positions and, in even more particular examples, bonded to the linker by way of a covalent bond between an atom on the linker and the carbon atom at the 5′ position of the thiophene. In such particular examples, the BRD9 binder may be of formula BRD91g′:

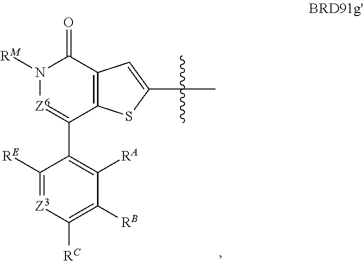

wherein the wavy line intersects the bond between the BRD9 binder and the linker; and
wherein R^{A}, R^{B}, R^{C}, R^{E}, R^{M}, Z³, and Z⁶ are as defined above.

In some embodiments, ring 1A is pyrrolidine. In particular examples, ring 1A is pyrrolidine fused to the rest of the BRD9 binder at the 3′ and 4′ positions and, in even more particular embodiments, bonded to the linker by way of a covalent bond between an atom on the linker and the nitrogen atom of the pyrrolidine. In such particular embodiments, the BRD9 binder may be of formula BRD91f′:

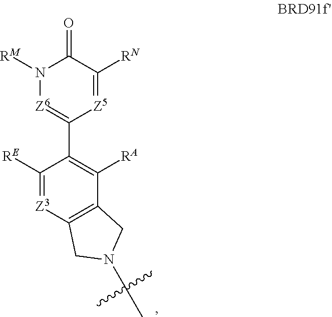

wherein the wavy line intersects the bond between the BRD9 binder and the linker; and
wherein R^{A}, R^{E}, R^{M}, R^{N}, Z³, and Z⁶ are as defined above and herein.

In some embodiments, the BRD9 binder is of formula BRD91e, BRD91f′ or BRD91g′.

In particular embodiments, the BRD9 binder is any one of formulae BRD91ea to BRD91eh, BRD91fa to BRD91fh and BRD91ga:

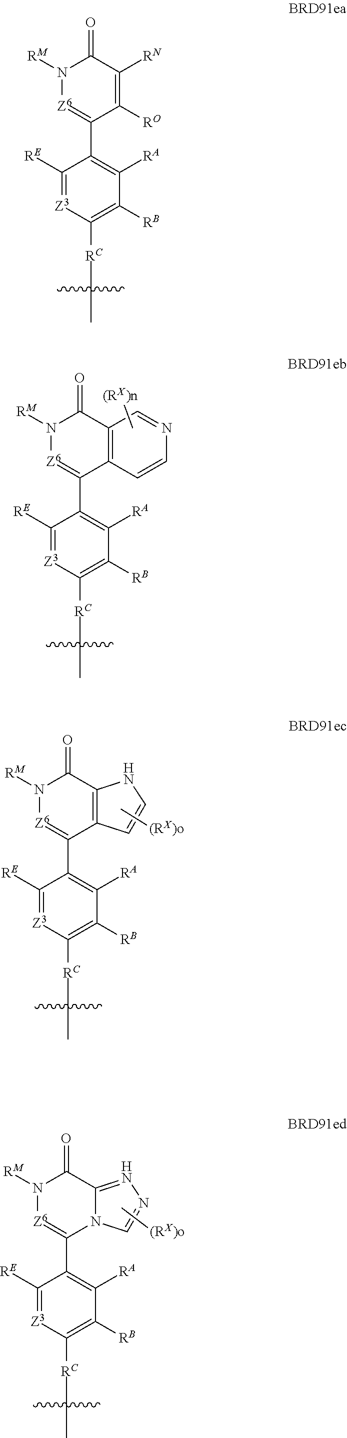

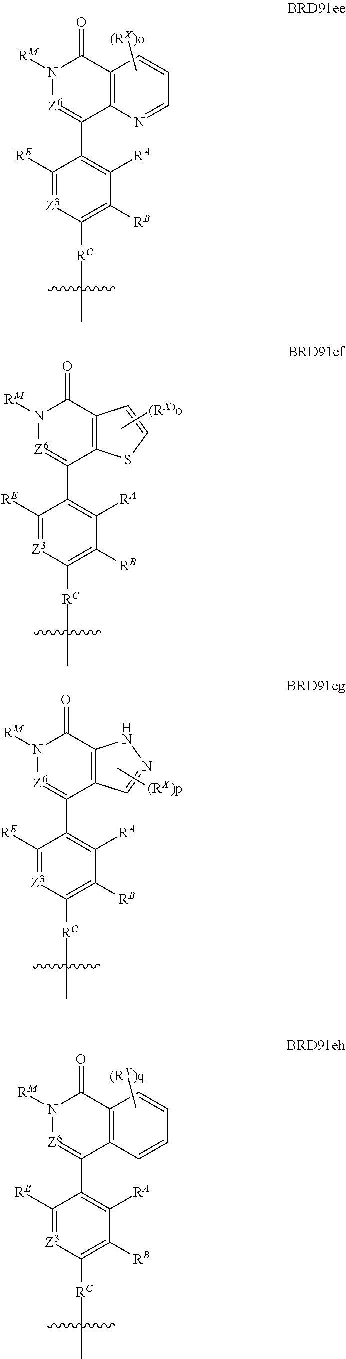

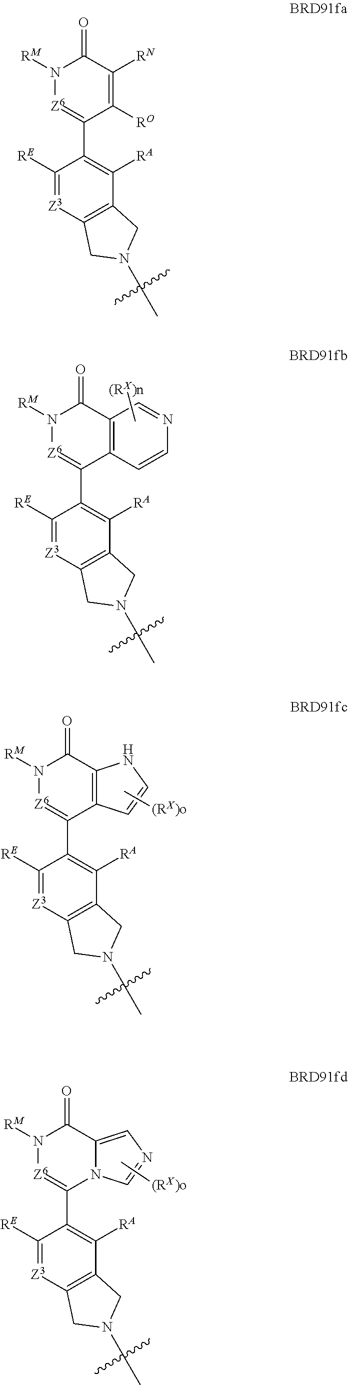

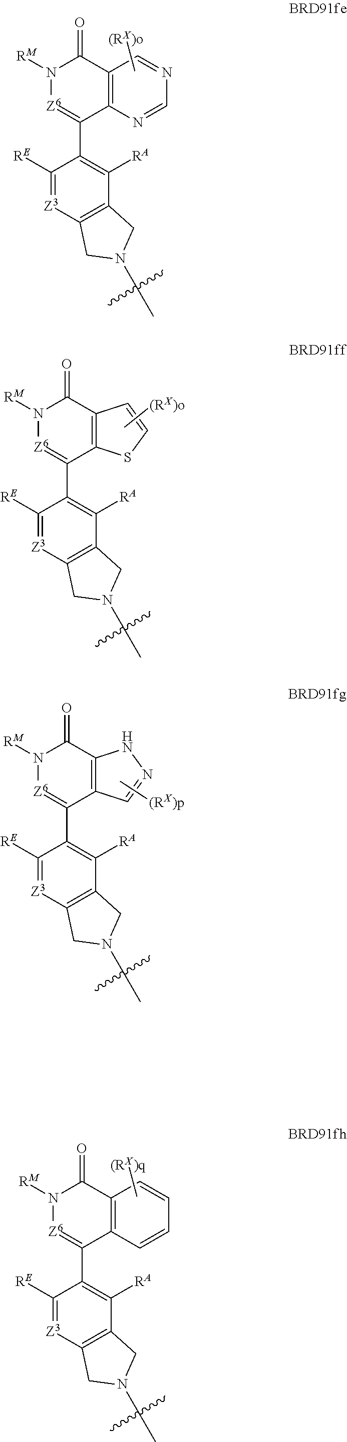

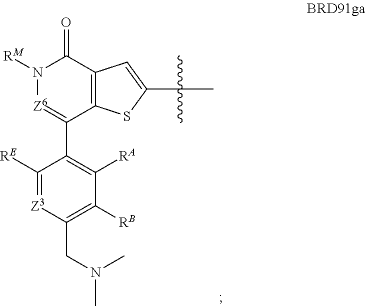

wherein the wavy line intersects the bond between the BRD9 binder and the linker;
R^{A}, R^{B}, R^{E}, R^{M}, Z³ and Z⁶ are as defined above and herein;
R^{C} is absent, or is as defined above and herein;
R^{N} is as defined above and herein, for example is selected from the group consisting of halogen, —C₁₋₅alkyl, —C₁₋₃haloalkyl, —H, C(O)C₁₋₅alkyl, —NH₂, —NHC₁₋₃alkyl and —OH;
R^{O} is as defined above and herein, for example is —H or —C₁₋₃alkyl;
each R^{X} is as defined for the optional substituents of the optionally substituted C₆₋₁₀aryl or optionally substituted C₂₋₉heteroaryl formed from R^{N} and Z⁵ (taken together), for example each R^{X} may be independently selected from the group consisting of halogen, —OH, —NH₂, —NH—C₁₋₃alkyl-C₁₋₅alkyl, C₁₋₅haloalkyl, C₁₋₅alkoxy and C₁₋₄haloalkoxy;
n is 0 to 3 (such as 0);
is 0 to 2 (such as 0);
p is 0 or 1 (such as 0); and
q is 0 to 4 (such as 0).

Each of n, o, p and q may be 0.

In some embodiments, the BRD9 binder is according to formula BRD91ea′:

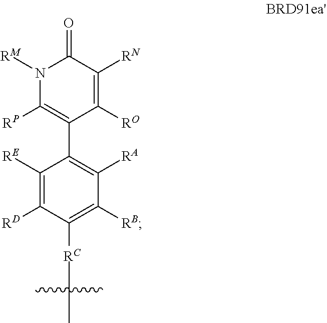

wherein the wavy line intersects the bond between the BRD9 binder and the linker;
R^{A} and R^{E} are as defined above and herein, for example are each independently selected from H and —O—C₁₋₃alkyl;
R^{B} and R^{D} are as defined above and herein, for example are each independently selected from —O—C₁₋₃alkyl, —H, -halo, —C₁₋₃alkyl, and —O—C₁₋₃haloalkyl;
R^{C} is absent, or is —Y—R^{G};
Y is selected from the group consisting of —CR^{H}R^{I}—, and —CO—;
R^{H} and R^{I} are each independently selected from —H or —C₁₋₃alkyl; or R^{H} and R^{I} taken together form a —C₃₋₄cycloalkyl;
R^{G} is selected from the group consisting of —N(R^{J}R^{K}) (e.g. —N(C₁₋₃alkyl)-, —N(C₁₋₃alkyl) (optionally substituted 4- to 7-membered monocyclic heterocycloalkylene), or —N(C₁₋₃alkyl) (optionally substituted 7- to 12-membered bicyclic heterocycloalkylene); —O—; optionally substituted 4- to 7-membered monocyclic heterocycloalkylene; and optionally substituted 7-to 12-membered bicyclic heterocycloalkylene;
R^{J} and R^{K} are as defined above and herein;
;
R^{M} is as defined above and herein, for example is C₁₋₃alkyl; and
R^{N}, R^{O} and R^{P} are each as defined above and herein, for example are each independently selected from the group consisting of halo, —C₁₋₃alkyl, and —C₁₋₃haloalkyl.

In even more particular embodiments, the BRD9 binder is any one of formulae BRD91 h to BRD91z and BRD92a to BRD92g:

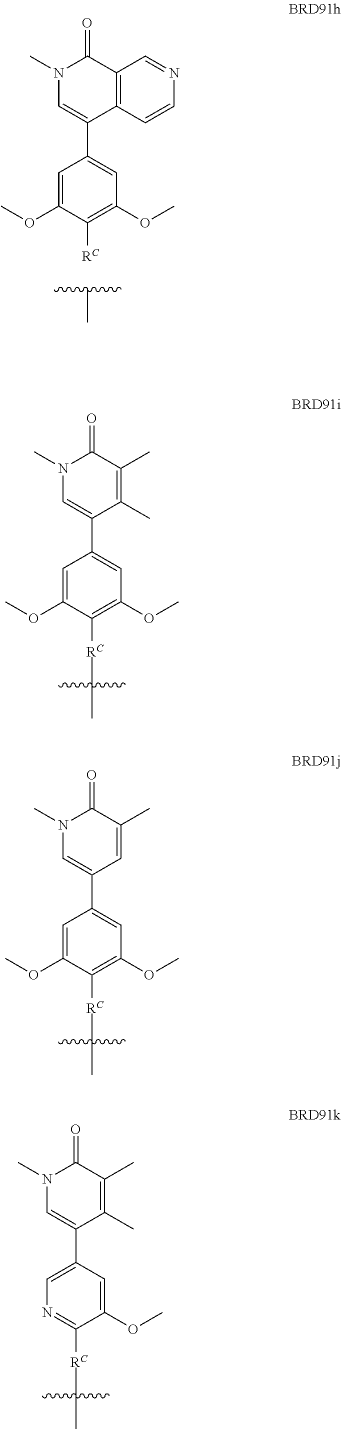

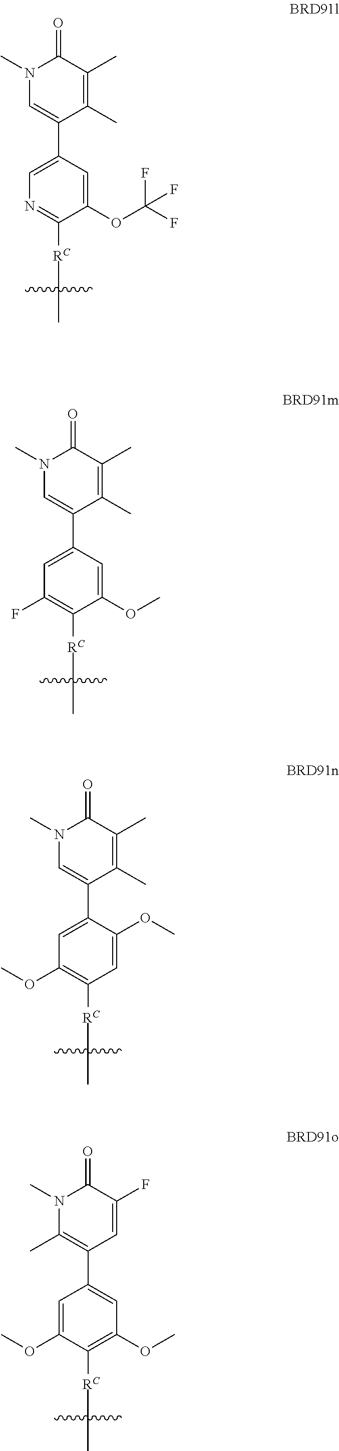

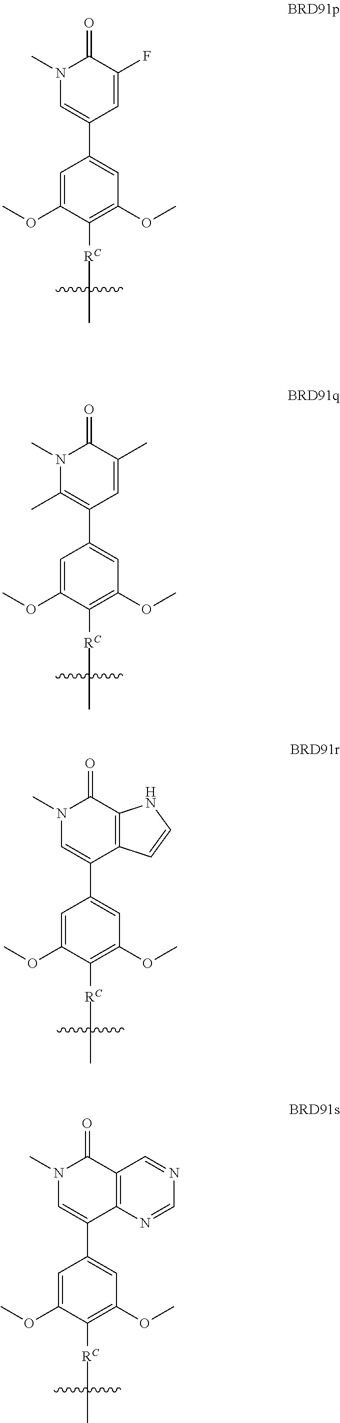

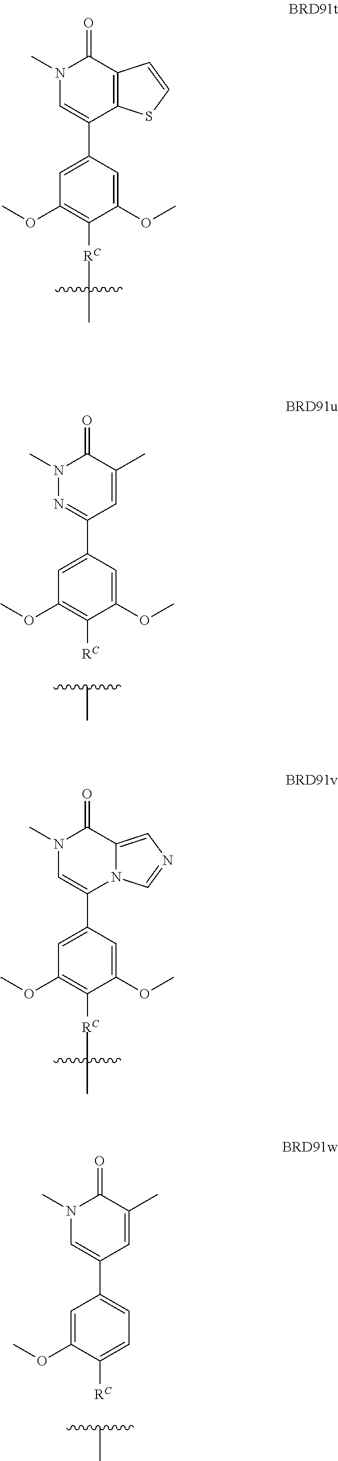

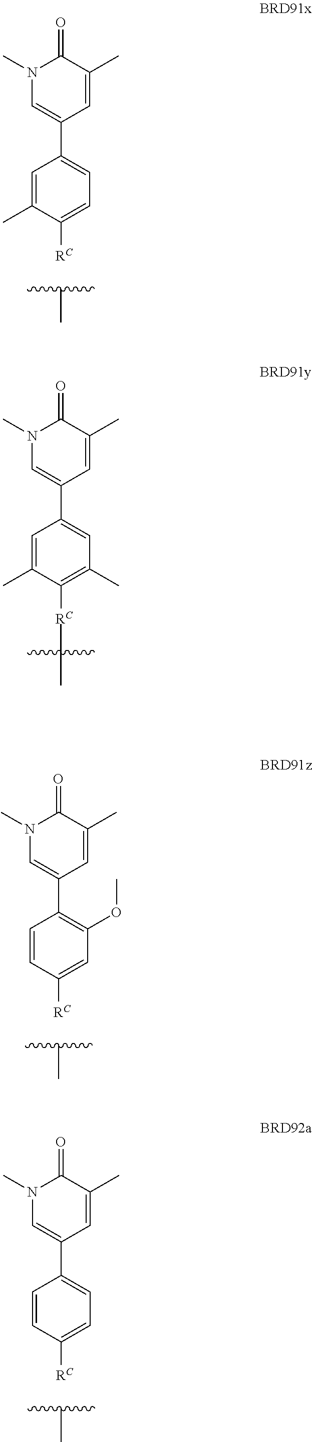

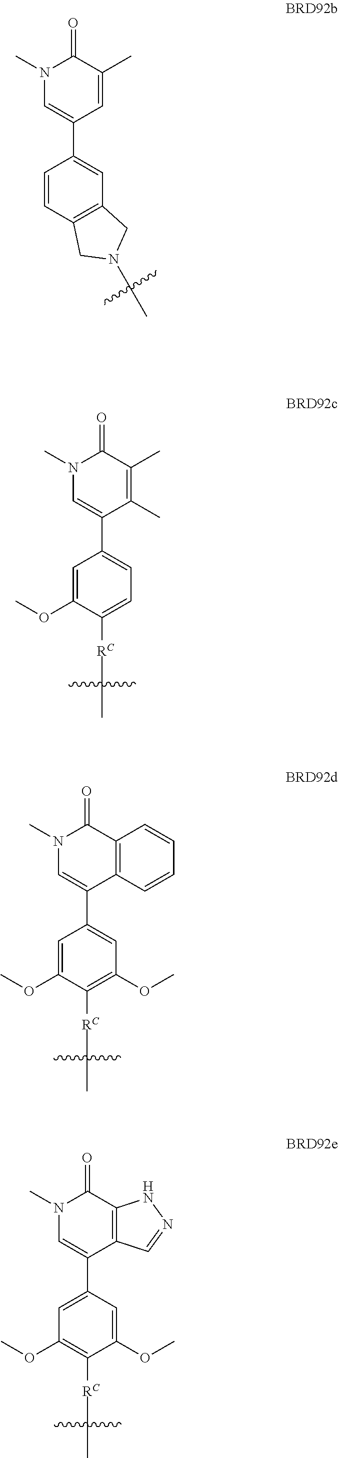

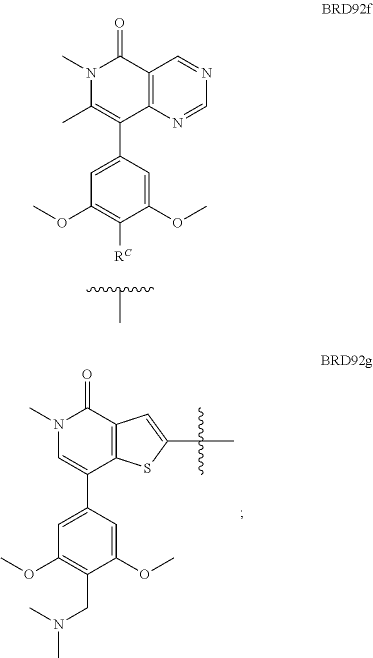

wherein R^{C} is absent, or is —Y—R^{G};
Y is selected from the group consisting of —CR^{H}R^{I}—, and —CO—;
R^{H} and R^{I} are each —H; or R^{H} and R^{I} taken together form a —C₃₋₄cycloalkyl;
R^{G} is selected from the group consisting of —N(R^{J}R^{K}) (e.g. —N(C₁₋₃alkyl)-, N(C₁₋₃alkyl) (optionally substituted 4- to 7-membered monocyclic heterocycloalkylene), or —N(C₁₋₃alkyl) (optionally substituted 7- to 12-membered bicyclic heterocycloalkylene) ); —O—; optionally substituted 4- to 7-membered monocyclic heterocycloalkylene containing one or two N ring atoms; and
optionally substituted 7- to 12-membered bicyclic heterocycloalkylene containing one or two N ring atoms;
R^{J} and R^{K} are as defined above and herein;
wherein the wavy line intersects the bond between the BRD9 binder and the linker.

In particular examples of any of the above formulae (e.g. any one of formulae BRD91e, BR91g, BRD91g′, BRD91ea to BRD91eh, BRD91ea′, BRD91 h to BRD91z and BRD92a to BRD92g, and unless otherwise stated), R^{G} is —N(C₁₋₃alkyl)-, —O— or

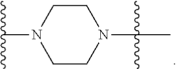 [SMILES: *N1CCN(*)CC1]

In some examples of any of the above formulae (e.g. any one of formulae BRD91e, BRD91g, BRD91g′, BRD91ea to BRD91eh, BRD91ea′, BRD91 h to BRD91z and BRD92a to BRD92g, and unless otherwise stated), R^{C} may be any one selected from:

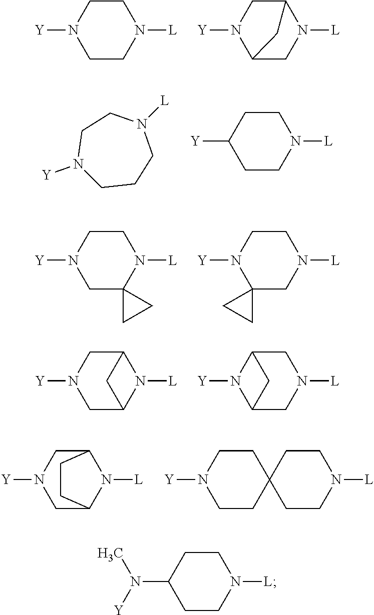

wherein Y is CR^{H}R_{I} (e.g. CH₂) or —CO—;
R^{H} and R^{I} are as defined above and herein; and
L shows the point of attachment of the linker.

In particular, in each of the structures shown above, Y may be CH₂.

In some cases, the BRD9 binder may not be:

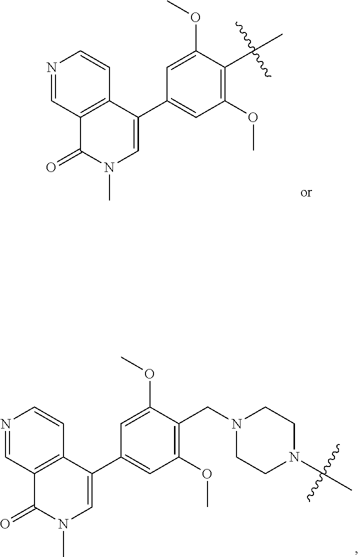 [SMILES: *c1c(OC)cc(-c2cn(C)c(=O)c3cnccc23)cc1OC ; *N1CCN(Cc2c(OC)cc(-c3cn(C)c(=O)c4cnccc34)cc2OC)CC1]

wherein the wavy line intersects the bond between the BRD9 binder and the linker.

### III. Heat Shock Protein 90 (HSP90) Inhibitors

HSP90 inhibitors useful according to the present disclosure include but are not limited to:

1. The HSP90 inhibitors identified in Vallee, et al., “Tricyclic Series of Heat Shock Protein 90 (HSP90) Inhibitors Part I: Discovery of Tricyclic Imidazo[4,5-C]Pyridines as Potent Inhibitors of the HSP90 Molecular Chaperone (2011,]. Med. Chem., 54:7206), including YKB (N-[4-(3H-imidazo[4,5-C]Pyridin-2-yl)-9H-Fluoren-9-yl]-succinamide):

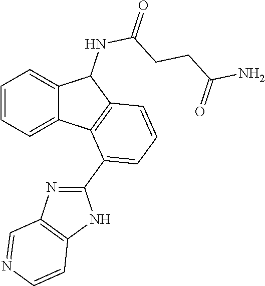 [SMILES: NC(=O)CCC(=O)NC1c2ccccc2-c2c(-c3nc4cnccc4[nH]3)cccc21]

derivatized where a linker is attached, for example, via the terminal amide group;

2. The HSP90 inhibitor p54 (modified) (8-[(2,4-dimethylphenyl)sulfanyl]-3]pent-4-yn-I-yl-3H-purin-6-amine):

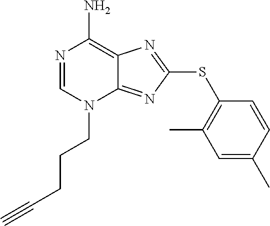 [SMILES: C#CCCCn1cnc(N)c2nc(Sc3ccc(C)cc3C)nc1-2]

where a linker is attached, for example, via the terminal acetylene group;

3. The HSP90 inhibitors (modified) identified in Brough, et al., “4,5-Diarylisoxazole HSP90 Chaperone Inhibitors: Potential Therapeutic Agents for the Treatment of Cancer”, (2008,]. Med. Chem., 51:196), including the compound 2GJ (5-[2,4-dihydroxy-5-(1-methylethyl)phenyl]-n-ethyl-4-[4-(morpholin-4-ylmethyl)phenyl] isoxazole-3-carboxamide) having the structure:

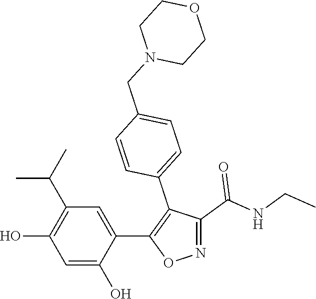 [SMILES: CCNC(=O)c1noc(-c2cc(C(C)C)c(O)cc2O)c1-c1ccc(CN2CCOCC2)cc1]

derivatized, where a linker is attached, for example, via the amide group (at the amine or at the alkyl group on the amine);

4. The HSP90 inhibitors (modified) identified in Wright, et al., Structure-Activity Relationships in Purine-Based Inhibitor Binding to HSP90 Isoforms, (2004 June., Chem Biol. 11(6):775-85), including the HSP90 inhibitor PU3 having the structure:

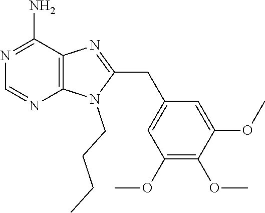 [SMILES: CCCCn1c(Cc2cc(OC)c(OC)c(OC)c2)nc2c(N)ncnc21]

where a linker group is attached, for example, via the butyl group; and

5. The HSP90 inhibitor geldanamycin ((4E,6Z,8S,9S,10E, 12S, 13R, 14S, 16R)-13-hydroxy-8, 14, 19-trimethoxy-4, 10, 12, 16-tetramethyl-3,20,22-trioxo-2-azabicyclo[16.3.I] (derivatized) or any its derivatives (e.g. 17-alkylamino-17-desmethoxygeldanamycin (“17-AAG”) or 17-(2-dimethylaminoethyl)amino-17-desmethoxygeldanamycin (“17-DMAG”)) (derivatized, where a is attached, for example, via the amide group).

### IV. HDM2/MDM2 Inhibitors

HDM2/MDM2 inhibitors of the invention include, but are not limited to:

1. The HDM2/MDM2 inhibitors identified in Vassilev, et al., In vivo activation of the p53 pathway by small-molecule antagonists of MDM2, (2004, Science, 303844-848), and Schneekloth, et al., Targeted intracellular protein degradation induced by a small molecule: En route to chemical proteomics, (2008, Biorg. Med. Chem. Lett., 18:5904-5908), including (or additionally) the compounds nutlin-3, nutlin-2, and nutlin-1 (derivatized) as described below, as well as all derivatives and analogs thereof:

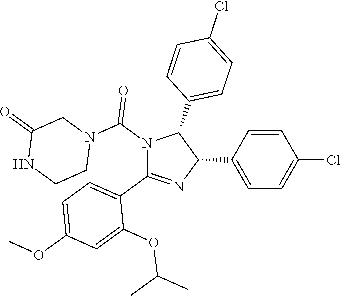 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

(derivatized where a linker is attached, for example, at the methoxy group or as a hydroxyl group);

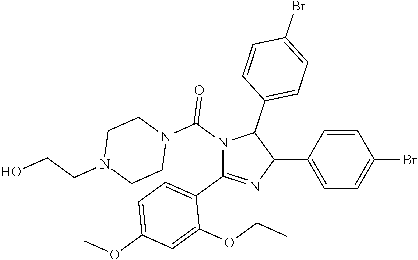 [SMILES: CCOc1cc(OC)ccc1C1=NC(c2ccc(Br)cc2)C(c2ccc(Br)cc2)N1C(=O)N1CCN(CCO)CC1]

(derivatized where a linker is attached, for example, at the methoxy group or hydroxyl group);

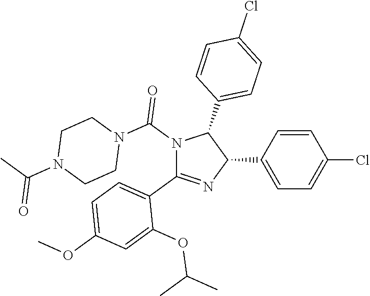 [SMILES: COc1ccc(C2=N[C@@H](c3ccc(Cl)cc3)[C@@H](c3ccc(Cl)cc3)N2C(=O)N2CCN(C(C)=O)CC2)c(OC(C)C)c1]

(derivatized where a linker is attached, for example, via the methoxy group or as a hydroxyl group); and

### V. HDAC Inhibitors

HDAC Inhibitors (derivatized) useful in some examples of the disclosure include, but are not limited to:
1. Finnin, M. S. et al. Structures of Histone Deacetylase Homologue Bound to the TSA and SAHA Inhibitors. (1999, Nature, 40:188-193).

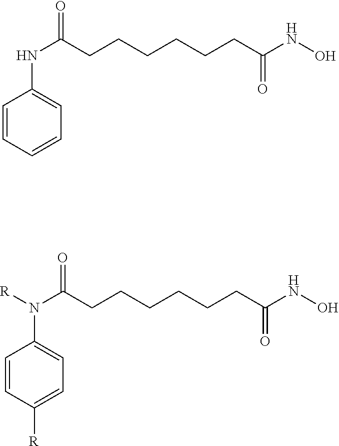

(Derivatized where “R” designates a site for attachment, for example, of a linker; and
2. Compounds as defined by formula (I) of PCT W00222577 (the entire contents of which are incorporated herein by reference) (“DEACETYLASE INHIBITORS”) (Derivatized where a linker is attached, for example, via the hydroxyl group);

### VI. Human Lysine Methyltransferase Inhibitors

Human Lysine Methyltransferase inhibitors useful in some examples of the disclosure include, but are not limited to:
1. Chang et al. Structural Basis for G9a-Like protein Lysine Methyltransferase Inhibition by BIX-1294 (2009, Nat. Struct. Biol., 16 (3): 312).

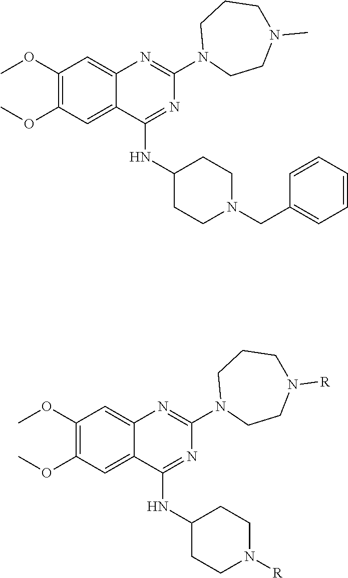

(Derivatized where “R” designates a site for attachment, for example, of a linker;
2. Liu, F. et al Discovery of a 2,4-Diamino-7-aminoalkoxyquinazoline as a Potent and Selective Inhibitor of Histone Methyltransferase G9a. (2009, J. Med. Chem., 52 (24): 7950).

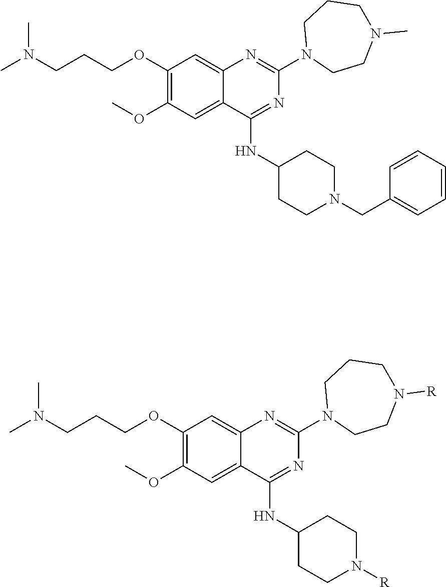

(Derivatized where “R” designates a potential site for attachment of a linker);
3. Azacitidine (derivatized) (4-amino-1-D-ribofuranosyl-1,3,5-triazin-2 (1H)-one) (Derivatized where a linker is attached, for example, via the hydroxy or amino groups); and
4. Decitabine (derivatized) (4-amino-1-(2-deoxy-b-D-erythro-pentofuranosyl)-1, 3, 5-triazin-2 (1H)-one) (Derivatized where a linker is attached, for example, via either of the hydroxy groups or at the amino group).

### VII. Angiogenesis Inhibitors

Angiogenesis inhibitors useful in some aspects of the disclosure include, but are not limited to:
1. GA-1 (derivatized) and derivatives and analogs thereof, having the structure(s) and binding to linkers as described in Sakamoto, et al., Development of Protacs to target cancer-promoting proteins for ubiquitination and degradation, (2003 December, Mol. Cell Proteomics, 2 (12): 1350-1358);
2. Estradiol (derivatized), which may be bound to a linker as is generally described in Rodriguez-Gonzalez, et al., Targeting steroid hormone receptors for ubiquitination and degradation in breast and prostate cancer, (2008, Oncogene 27:7201-7211);
3. Estradiol, testosterone (derivatized) and related derivatives, including but not limited to DHT and derivatives and analogs thereof, having the structure(s) and binding to a linker as generally described in Sakamoto, et al., Development of Protacs to target cancer-promoting proteins for ubiquitination and degradation, (2003 December, Mol. Cell Proteomics, 2 (12): 1350-1358); and
4. Ovalicin, fumagillin (derivatized), and derivatives and analogs thereof, having the structure(s) and binding to a linker as is generally described in Sakamoto, et al., Protacs: chimeric molecules that target proteins to the Skp1-Cullin-F box complex for ubiquitination and degradation (2001 July, Proc. Natl. Acad. Sci. USA, 98 (15): 8554-8559) and U.S. Pat. No. 7,208,157, the entire contents of which are incorporated herein by reference.

### VIII. Immunosuppressive Compounds

Immunosuppressive compounds useful in some examples of the disclosure include, but are not limited to:
1. AP21998 (derivatized), having the structure(s) and binding to a linker as is generally described in Schneekloth, et al., Chemical Genetic Control of Protein Levels: Selective in Vivo Targeted Degradation (2004, J. Am. Chem. Soc., 126:3748-3754);
2. Glucocorticoids (e.g., hydrocortisone, prednisone, prednisolone, and methylprednisolone) (Derivatized where a linker is bound, e.g. to any of the hydroxyls) and beclometasone dipropionate (Derivatized where a linker is bound, e.g. to a proprionate);
3. Methotrexate (Derivatized where a linker can be bound, e.g. to either of the terminal hydroxyls);
4. Ciclosporin (Derivatized where a linker can be bound, e.g. at a of the butyl groups);
5. Tacrolimus (FK-506) and rapamycin (Derivatized where a linker group can be bound, e.g. at one of the methoxy groups); and
6. Actinomycins (Derivatized where a linker can be bound, e.g. at one of the isopropyl groups).

### IX. Compounds Targeting the Aryl Hydrocarbon Receptor (AHR)

Compounds targeting the aryl hydrocarbon receptor (AHR) according to some examples of the disclosure include, but are not limited to:
1. Apigenin (Derivatized in a way which binds to a linker as is generally illustrated in Lee, et al., Targeted Degradation of the Aryl Hydrocarbon Receptor by the PROTAC Approach: A Useful Chemical Genetic Tool, ChemBioChem Volume 8, Issue 17, pages 2058-2062 Nov. 23, 2007); and
2. SRI and LGC006 (derivatized such that a linker is bound), as described in Boitano, et al., Aryl Hydrocarbon Receptor Antagonists Promote the Expansion of Human Hematopoietic Stem Cells (2010 September, Science, 329 (5997): 1345-1348).

### XI. Compounds Targeting FKBP

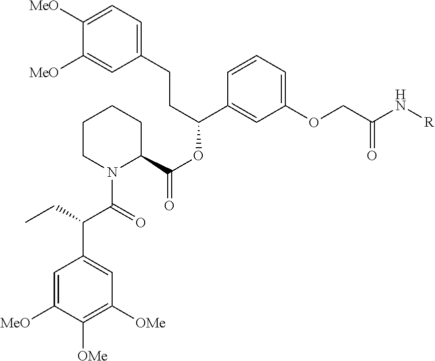

(Derivatized where “R” designates a site for linker attachment).

### XIV. Compounds Targeting Thyroid Hormone Receptor (TR)

1. Thyroid Hormone Receptor Ligand (derivatized)

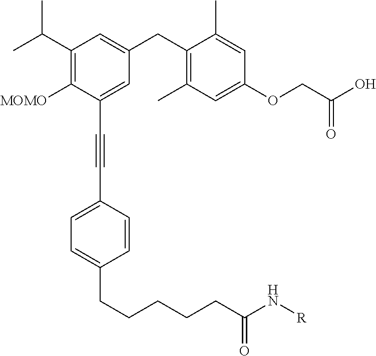

(Derivatized where “R” designates a site for linker attachment and MOMO indicates a methoxymethoxy group).

### XV. Compounds Targeting HIV Protease

1. Inhibitor of HIV Protease (derivatized)

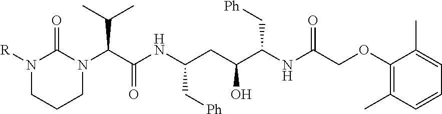

(Derivatized where “R” designates a site for linker attachment). See, 2010, J. Med. Chem., 53:521-538.
2. Inhibitor of HIV Protease

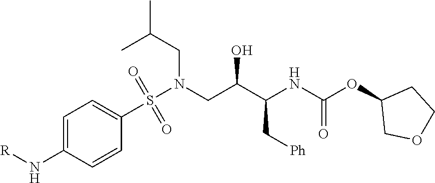

(Derivatized where “R” designates a potential site for linker attachment). See, 2010, J. Med. Chem., 53:521-538.

### XVI. Compounds Targeting HIV Integrase

1. Inhibitor of HIV Integrase (derivatized)

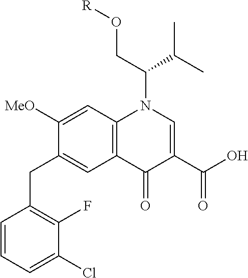

(Derivatized where “R” designates a site for linker attachment). See, 2010, J. Med. Chem., 53:6466.
2. Inhibitor of HIV Integrase (derivatized)

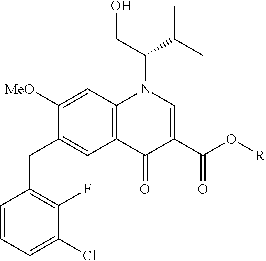

3. Inhibitor of HIV integrase Isentress (derivatized)

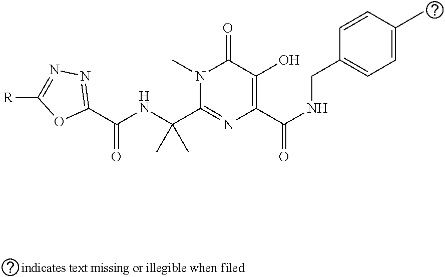

(Derivatized where “R” designates a site for linker attachment). See, 2010, J. Med. Chem., 53:6466.

### XVII. Compounds Targeting HCV Protease

1. Inhibitors of HCV Protease (derivatized)

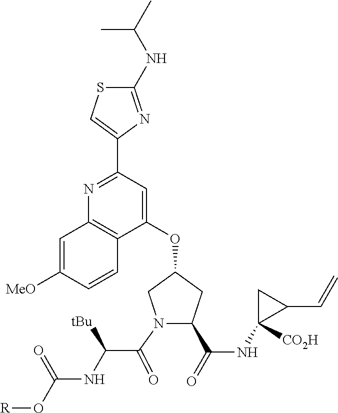

(Derivatized where “R” designates a site for linker attachment).

### XVIII. Compounds Targeting Acyl-Protein Thioesterase-1 and -2 (APTI and APT2)

1. Inhibitor of APTI and APT2 (derivatized)

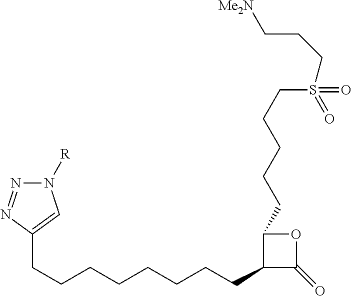

(Derivatized where “R” designates a site for linker attachment). See 2011, Angew. Chem. Int. Ed.,50:9838-9842.

### XIX. Compounds Targeting Ubiquitin-Specific Protease 1 (Usp1)

Additional examples of USP1 binding ligands are provided below.

A compound of formula USP1A, USP2A or USP3A:

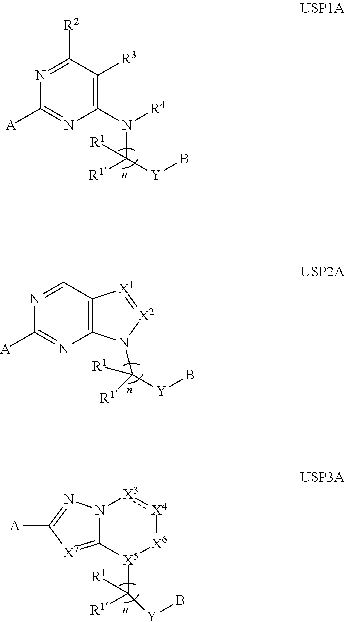

wherein:
A is an aryl or heteroaryl, each of which is optionally substituted with any one or more substituents selected from the group consisting of C₁₋₄alkyl, C₃₋₇ cycloalkyl, C₁₋₄alkoxy, C₁₋₄ haloalkyl, C₃₋₇ cyclohaloalkyl, C₁₋₄ haloalkoxy, halo, hydroxyl and amino;
B is a heteroaryl comprising at least one N ring atom, optionally substituted with any one or more substituents selected from the group consisting of C₁₋₄alkyl, C₁₋₄ haloalkyl, C₃₋₇ cycloalkyl, C₁₋₄ alkoxy, C₃₋₇ cyclohaloalkyl, C₁₋₄ haloalkoxy, halo, hydroxyl and amino;
R¹ and R¹′ are each independently selected from H and C₁₋₂ alkyl;
n is 1 to 3;
Y is an aryl or heteroaryl, each of which is optionally substituted with any one or more selected from the group consisting of C₁₋₄alkyl, C₃₋₇ cycloalkyl, C₁₋₄alkoxy, C₁₋₄ haloalkyl, C₃₋₇ cyclohaloalkyl, C₁₋₄ haloalkoxy, halo, hydroxyl and amino;
R² and R⁴ are each independently selected from the group consisting of H and C₁₋₄ alkyl;
R³ is selected from the group consisting of L-Z or C₁₋₄ alkyl-L-Z;
X¹ and X² are each independently selected from the group consisting of N, C-L-Z and C—R⁵, where R⁵ is selected from the group consisting of H and C₁-C₄ alkyl; and wherein one of X¹ and X² is C-L-Z;
wherein the bond shown as [symbol] indicates that the bond is a single or double bond;
wherein when the bond is a double bond, X³ and X⁴ are independently selected from the group consisting of CL-Z and CR⁶; and wherein one of X³ and X⁴ is C-L-Z; and
wherein when the bond is a single bond, X³ and X⁴ are independently selected from the group consisting of CR⁶L-Z and CR⁶₂, and wherein one of X³ and X⁴ is CR⁶L-Z;
wherein each R⁶ is independently selected from the group consisting of H C₁₋₄ alkyl, NH₂, NHMe and NMe₂;
X⁵ is N or CH;
X⁶ is selected from C═O, CR⁷₂, NH and NMe, where each R⁷ is independently selected from the group consisting of H and C₁₋₄ alkyl;
X⁷ is N or CH;
L is a linker (as defined above); and
Z is as defined above and herein.

In some cases, the compound targeting Usp1 is a compound of formula USP1, USP2, or USP3:

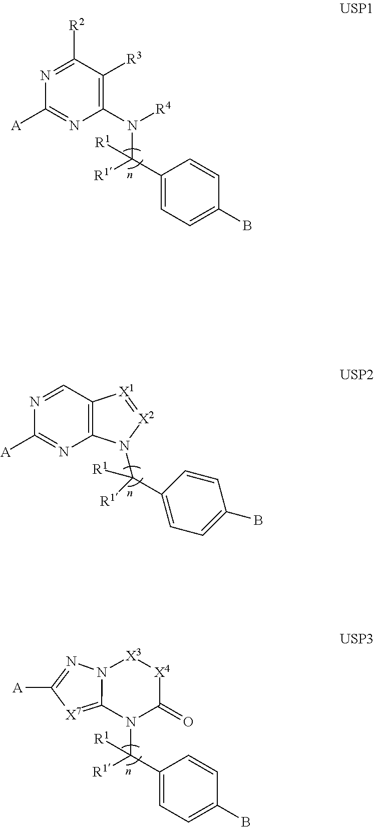

wherein:
A is an aryl or heteroaryl, each of which is optionally substituted with any one or more selected from the group consisting of C₁₋₄alkyl, C₃₋₇ cycloalkyl, C₁₋₄alkoxy, C₁₋₄ haloalkyl, C₃₋₇ cyclohaloalkyl, C₁₋₄ haloalkoxy, halo, hydroxyl and amino;
B is a heteroaryl comprising at least one N ring atom, optionally substituted with any one or more selected from the group consisting of C₁₋₄alkyl, C₁₋₄ haloalkyl, C₃₋₇ cycloalkyl, C₁₋₄ alkoxy, C₃₋₇ cyclohaloalkyl, C₁₋₄ haloalkoxy, halo, hydroxyl and amino;
R¹ and R¹′ are each independently selected from H and C₁₋₂ alkyl;
n is 1 to 3;
R² and R⁴ are each independently selected from the group consisting of H and C₁₋₄ alkyl;
R³ is selected from the group consisting of L-Z or C₁₋₄ alkyl-L-Z;
X¹ and X² are each independently selected from the group consisting of N, C-L-Z and C—R⁵, where R⁵ is selected from the group consisting of H and C₁-C₄ alkyl; and wherein one of X¹ and X² is C-L-Z;
X³ and X⁴ are independently selected from the group consisting of CR⁶L-Z and CR⁶₂, where each R⁶ is independently selected from the group consisting of H and C₁₋₄ alkyl; and wherein one of X³ and X⁴ is CR⁶L-Z;
X⁷ is N or CH;
L is a linker (as defined above and herein); and
Z is as defined above and herein.

By way of further example, the compound targeting Usp1 may comprise a compound of formula USP1a, USP2a, or USP3a:

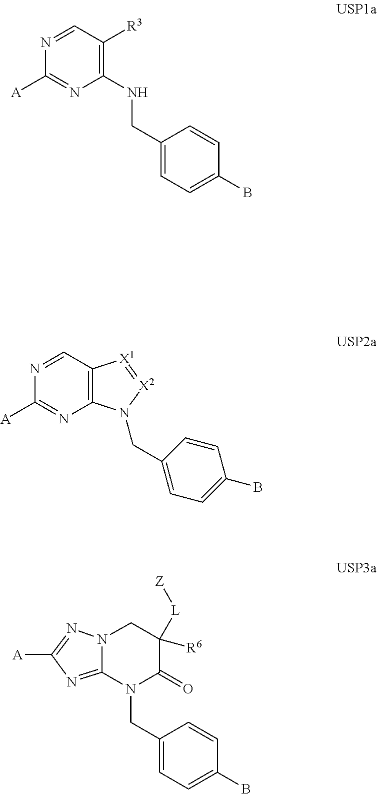

wherein A, B, R³, X¹, X², R⁶, L and Z are as defined above for formula USP1A, USP2A, USP3A, USP1, USP2, and USP3.

Representative examples of groups A, B, Y, X¹, X², X³, X⁴, R¹, R¹′, R², R³, R₄, R⁵ and R⁶ are now provided below which are applicable to any one or more of the formulae described herein in relation to compounds targeting Usp1 (unless otherwise indicated) (formulae USP1A, USP2A, USP3A, USP1, USP2, USP3, USP1a, USP2a, and USP3a).

As stated above, A may be an aryl or heteroaryl, each of which is optionally substituted with any one or more selected from the group consisting of C₁₋₄alkyl, C₃₋₇ cycloalkyl, C₁₋₄alkoxy, C₁₋₄ haloalkyl, C₃₋₇ cyclohaloalkyl, C₁₋₄ haloalkoxy, halo, hydroxyl and amino. For the avoidance of doubt, where A is a heteroaryl, it is optionally substituted at one or more carbon or heteroatoms.

Where A is a heteroaryl, it may be contain one or more nitrogen atoms, such as two nitrogen atoms. Sometimes, the heteroaryl is monocyclic. Sometimes the heteroaryl is a N-heteroaryl.

In some examples, A is selected from the group consisting of phenyl, pyridinyl, pyrimidinyl, pyrazinyl, pyrazolyl, pyridazinyl, optionally substituted with any one or more substituents selected from the group consisting of C₁₋₄alkyl, C₃₋₇ cycloalkyl, C₁₋₄alkoxy, C₁₋₄ haloalkyl, C₃₋₇ cyclohaloalkyl, C₁₋₄ haloalkoxy, halo, hydroxyl and amino.

By way of further example, A may be selected from the group consisting of phenyl, pyrimidinyl and pyrazolyl, optionally substituted with any one or more substituents selected from the group consisting of C₁₋₄alkyl (e.g. isopropyl or methyl), C₃₋₇ cycloalkyl (e.g. cyclopropyl), C₁₋₄alkoxy (e.g. methoxy), C₁₋₄ haloalkyl (e.g. C₁₋₄ fluoroalkyl, such as trifluoromethyl), C₃₋₇ cyclohaloalkyl (e.g. C₃₋₇ cyclofluoroalkyl), C₁₋₄ haloalkoxy (e.g. C₁₋₄ fluoroalkoxy, such as difluoromethoxy) and halo (e.g. chloro or fluoro, such as chloro). By way of even further example, A may be selected from the group consisting of phenyl, pyrimidinyl and pyrazolyl, optionally substituted with any one or two substituents selected from the group consisting of C₁₋₄alkyl (e.g. isopropyl or methyl), C₃₋₇ cycloalkyl (e.g. cyclopropyl), C₁₋₄alkoxy (e.g. methoxy), C₁₋₄ haloalkoxy (e.g. difluoromethoxy) and halo (e.g. chloro). Sometimes, A is selected from the group consisting of phenyl and pyrimidinyl, optionally substituted with any one or two substituents selected from the group consisting of C₁₋₄alkyl (e.g. isopropyl), C₃₋₇ cycloalkyl (e.g. cyclopropyl), and C₁₋₄alkoxy (e.g. methoxy). In some examples, A may be selected from N-alkyl 4-alkyl pyrazolyl, 4,6-dialkyl pyrimidinyl and, 2,4-dialkyl pyridinyl (where each of the alkyl groups are C₁₋₄alkyl).

Representative examples of suitable A groups include, but are not limited to:

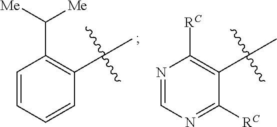

wherein R^{C} is selected from the group consisting of methoxy, cyclopropyl, and difluoromethoxy.

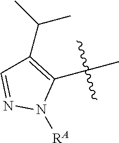

where R^{A} is methyl, ethyl, fluoro or difluorotrifluoroethyl.

In the structures shown above, the line intersected by a wavy line represents the covalent bond between the exemplary A groups shown above and a carbon atom on the heteroaryl core attached to the A group (as illustrated by the various formulae described herein). Although a particular substitution pattern is shown in the exemplary aryl and heteroaryl structures above, it will be appreciated that other substitution patterns are also encompassed within the scope of the present disclosure.

As stated above, B is a heteroaryl comprising at least one N ring atom, optionally substituted with any one or more selected from the group consisting of C₁₋₄alkyl, C₁₋₄ haloalkyl, C₃₋₇ cycloalkyl, C₁₋₄ alkoxy, C₃₋₇ cyclohaloalkyl, C₁₋₄ haloalkoxy, halo, hydroxyl and amino. For the avoidance of doubt, B is optionally substituted at one or more carbon or heteroatoms.

In some examples, B is a monocyclic five- or six-membered heteroaryl comprising at least one N ring atom (e.g. comprising 1, 2 or 3 N ring atoms). By way of further example, B may be selected from the group consisting of pyrrolyl, pyrazolyl, imidazolyl, thiazolyl, triazolyl, oxazolyl, isoxazolyl, and oxadiazolyl, optionally substituted with any one or more substituents selected from the group consisting of C₁₋₄alkyl, C₁₋₄ haloalkyl, C₃₋₇ cycloalkyl, C₁₋₄ alkoxy, C₃₋₇ cyclohaloalkyl, C₁₋₄ haloalkoxy, halo, hydroxyl and amino. Often, B is a monocyclic five-membered heteroaryl. Sometimes, B comprises at least 2 nitrogen atoms. By way of further example, B may be selected from the group consisting of imidazolyl, pyrazolyl and triazolyl (e.g. 1, 2, 3-triazolyl), optionally substituted with any one or more substituents selected from the group consisting of C₁₋₄alkyl, C₁₋₄ haloalkyl, C₃₋₇ cycloalkyl, C₁₋₄ alkoxy, C₃₋₇ cyclohaloalkyl, C₁₋₄ haloalkoxy, and halo. In some examples, B may be selected from the group consisting of imidazolyl, pyrazolyl and triazolyl (e.g. 1, 2, 3-triazolyl), optionally substituted with any one or more substituents selected from the group consisting of C₁₋₄alkyl (e.g. isopropyl, methyl or ethyl), C₁₋₄ haloalkyl (e.g. trifluoromethyl or fluoroethyl), and C₁₋₄ alkoxy (such as methoxy).

Representative examples of suitable B groups include, but are not limited to:

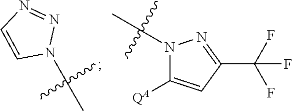

where Q^{A} is selected from methyl, isopropyl, ethyl, fluoroethyl and methoxy;

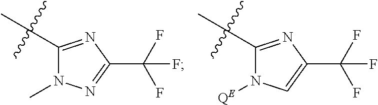

where Q^{E} is selected from isopropyl, methyl, ethyl, and fluoroethyl.

In the structures shown above, the line intersected by a wavy line represents the covalent bond between the exemplary B groups shown above and a carbon atom on the aryl core attached to the B group (as illustrated by the various formulae described herein). Although a particular substitution pattern is shown in the exemplary heteroaryl structures above, it will be appreciated that other substitution patterns are also encompassed within the scope of the present disclosure.

As stated above, Y may be an aryl or heteroaryl, each of which is optionally substituted with any one or more selected from the group consisting of C₁₋₄alkyl, C₃₋₇ cycloalkyl, C₁₋₄alkoxy, C₁₋₄ haloalkyl, C₃₋₇ cyclohaloalkyl, C₁₋₄ haloalkoxy, halo, hydroxyl and amino.

For the avoidance of doubt, Y is a bi-radical linking B with CR¹R¹′. References to any monoradical species in relation to Y are intended to mean bi-radical species able to link B with CR¹R¹′. For example, reference to phenyl means a biradical benzene group linking B with CR¹R¹.

Often, when Y is phenyl, B and CR¹R¹′ are positioned para to one another.

Often, when Y is a heteroaryl, it is a monocyclic 6-membered heteroaryl. The heteroaryl is typically a N-heteroaryl comprising at least one nitrogen atom. Often, the N-heteroaryl comprises two nitrogen atoms.

By way of example, Y may be phenyl, pyrimidinyl (such as pyrimidin-1,5-diyl), or pyridinyl, optionally substituted with any one or more substituents selected from the group consisting of halo and C₁₋₄alkoxy.

Representative examples of Y include, but are not limited to:

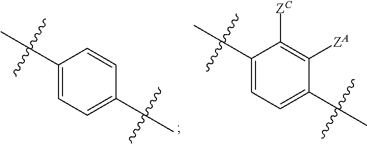

where Z^{A} is selected from H and halo (e.g. F); and Z^{C} is selected from H and methoxy; and

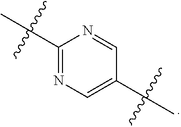 [SMILES: *c1cnc(*)nc1]

As stated above, R¹ and R¹′ are each independently selected from H and C₁₋₂ alkyl (e.g. methyl). By way of example, R¹ and R¹′ may each be H.

As stated above, n may be 1 to 3. In some examples, n is 1.

As stated above, R² and R⁴ are each independently selected from the group consisting of H and C₁₋₄ alkyl (e.g. methyl). By way of example, R² and R⁴ may both be H.

In those examples, where n is 1, and each of R¹, R¹′, R² and R⁴ is H, the bifunctional molecule may comprise a compound of formula USP1a, USP2a or USP3a:

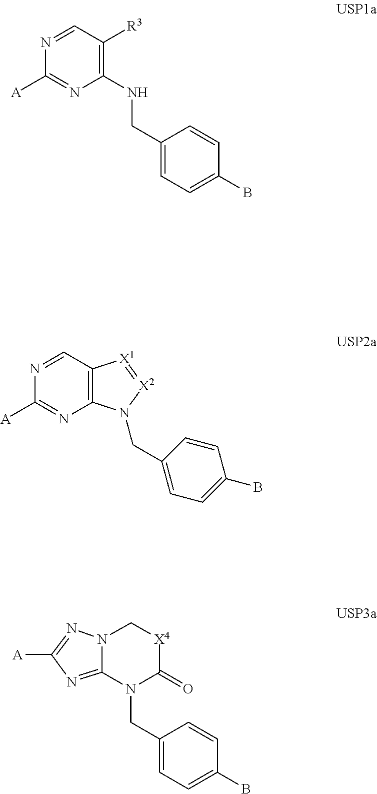

wherein A, B, R³, X¹, X² and X⁴ are as defined above (and herein) for formula USP1, USP2 and USP3.

In relation to formula USP2 (and also formula (USP2a), as stated previously), X¹ and X² are each independently selected from the group consisting of N, C-L-Z and C—R⁵, where R⁵ is selected from the group consisting of H and C₁-C₄ alkyl; and wherein one of X¹ and X² is C-L-Z.

In those cases where X¹ is C-L-Z, X² may be N or C—R⁵, wherein R⁵ is selected from the group consisting of H and C₁-C₄ alkyl (e.g. methyl). In some examples, where X¹ is C-L-Z, X² may be CH.

In those cases where X² is C-L-Z, X¹ may be N or C—R⁵, wherein R⁵ is selected from the group consisting of H and C₁-C₄ alkyl (e.g. methyl). In some examples, where X² is C-L-Z, X² may be CH.

In some examples of the disclosure, the bifunctional molecules comprise a compound of formula USP2b, USP2c, USP2d or USP2e:

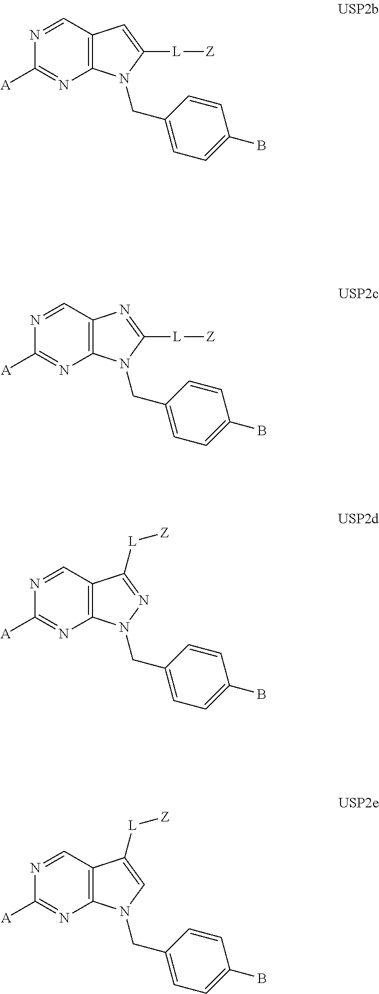

wherein A, B, L and Z are as defined above and herein in respect of the Usp target binding ligand.

In relation to formula USP3, X³ and X⁴ are independently selected from the group consisting of CR⁶L-Z and CR⁶₂, where each R⁶ is independently selected from the group consisting of H and C₁₋₄ alkyl; and wherein one of X³ and X⁴ is CR⁶L-Z.

In those examples where X³ is CR⁶L-Z, X⁴ may be CR⁶₂, wherein each R⁶ is independently selected from the group consisting of H and C₁₋₄ alkyl (e.g. methyl). By way of example, X³ may be CH-L-Z and X⁴ may be CH₂.

In those examples where X⁴ is CR⁶L-Z, X³ may be CR⁶₂, wherein each R⁶ is independently selected from the group consisting of H and C₁₋₄ alkyl (e.g. methyl). By way of example, X⁴ may be CH-L-Z and X³ may be CH₂.

In some examples of the disclosure, compound targeting Usp1 is a compound of formula USP3b, USP3c, USP3d or USP3e:

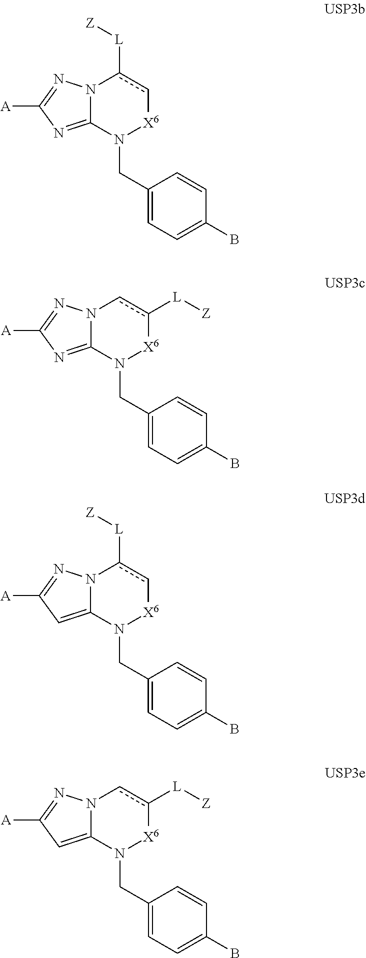

wherein A, B, X⁶, [symbol] L and Z are as defined above and herein.

In particular, in some examples, X⁶ of any of formula USP3b to USP3e, is C═O.

Further representative examples of compounds targeting Usp1 in accordance with the disclosure are illustrated below.

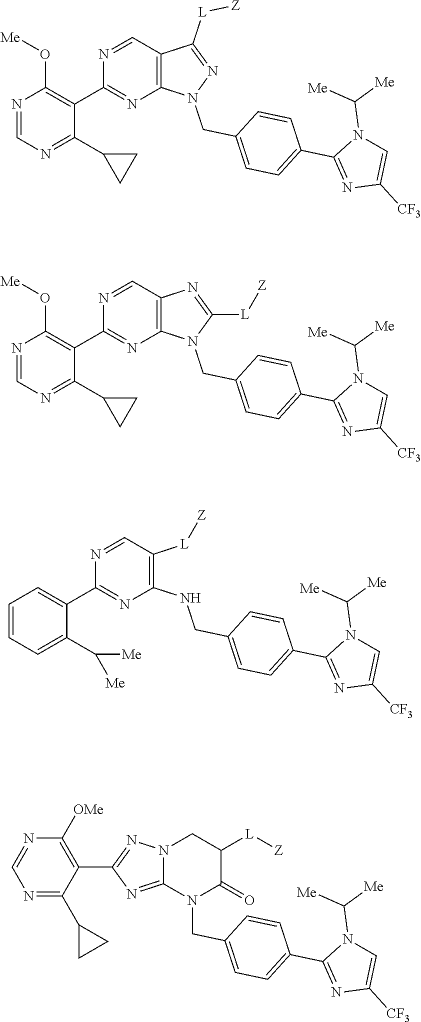

wherein L and Z are as defined above and herein.

### XX. Compounds Targeting Aggregation-Prone Proteins (Including Such as Binders to Synuclein)

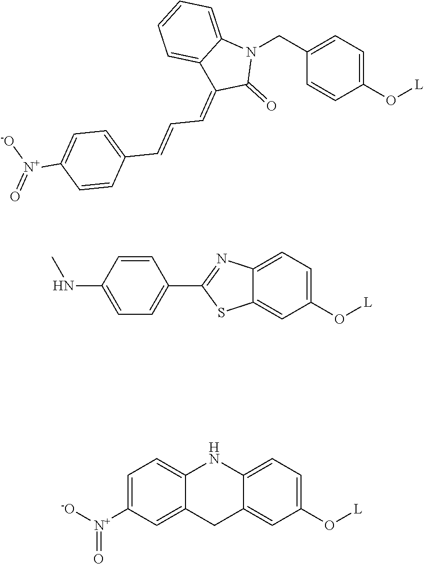

where L shows the position of attachment of the linker. The present disclosure also encompasses joining or coupling to the linker at any other chemically suitable position on this target protein binding ligand.

### XXI. Compounds Targeting Apoptotic & Anti-Apoptotic Factors (Including Bcl2 and Bcl-XL)

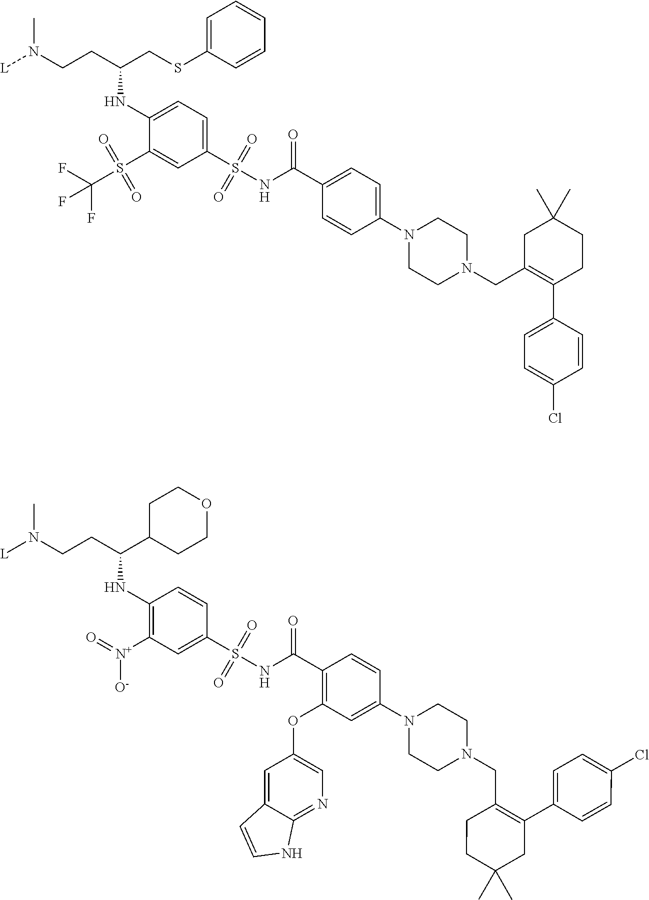

where L shows the position of attachment of the linker. The present disclosure also encompasses joining or coupling to the linker at any other chemically suitable position on this target protein binding ligand.

### Isotopically-Labelled Compounds

The disclosure also includes various deuterated forms of the compounds disclosed herein, or of any of the Formulae disclosed herein, including Formulae (ZI), (ZII), (ZIIIa to ZIIIf), (ZIVa to ZIVj), (I), (II) (III), (IV), (IVa), (ZV), (V), (VI) and (VIa); (EGFR1), (EGFR2) and (EGFR3); (KRAS1), (KRAS2), and (KRAS3); (C1) to (C12); (BRD91), 1T, 2T, 3T, 4T, 5T, 6T, 7T, 8T, 9T, 10T, 11T, 12T, 13T, 14T or (USP1A), (USP2A), and (USP3A) (inc. corresponding subgeneric formulae defined herein), respectively, or a pharmaceutically acceptable salt and/or a corresponding tautomer form thereof (including subgeneric formulas, as defined above) of the present disclosure. Each available hydrogen atom attached to a carbon atom may be independently replaced with a deuterium atom. A person of ordinary skill in the art will know how to synthesize deuterated forms of the compounds of any of the Formulae disclosed herein, including Formulae (ZI), (ZII), (ZIIIa to ZIIIf), (ZIVa to ZIVj), (I), (II) (III), (IV), (IVa), (ZV), (V), (VI) and (VIa); (EGFR1), (EGFR2) and (EGFR3); (KRAS1), (KRAS2), and (KRAS3); (C1) to (C12); (BRD91), 1T, 2T, 3T, 4T, 5T, 6T, 7T, 8T, 9T, 10T, 11T, 12T, 13T, 14T or (USP1A), (USP2A), and (USP3A) (inc. corresponding subgeneric formulae defined herein), respectively, or a pharmaceutically acceptable salt and/or a corresponding tautomer form thereof (including subgeneric formulae, as defined above) of the present disclosure. For example, deuterated materials, such as alkyl groups may be prepared by conventional techniques (see for example: methyl-d₃-amine available from Aldrich Chemical Co., Milwaukee, WI, Cat. No.489,689-2).

The disclosure also includes isotopically-labelled compounds which are identical to those recited in any of the Formulae disclosed herein, including Formulae (ZI), (ZII), (ZIIIa to ZIIIf), (ZIVa to ZIVj), (I), (II) (III), (IV), (IVa), (ZV), (V), (VI) and (VIa); (EGFR1), (EGFR2) and (EGFR3); (KRAS1), (KRAS2), and (KRAS3); (C1) to (C12); (BRD91), 1T, 2T, 3T, 4T, 5T, 6T, 7T, 8T, 9T, 10T, 11T, 12T, 13T, 14T or (USP1A), (USP2A), and (USP3A) (inc. corresponding subgeneric formulae defined herein), respectively, or a pharmaceutically acceptable salt and/or a corresponding tautomer form thereof (including subgeneric formulae, as defined above) of the present disclosure but for the fact that one or more atoms are replaced by an atom having an atomic mass or mass number different from the atomic mass or mass number most commonly found in nature. Examples of isotopes that can be incorporated into compounds of the disclosure include isotopes of hydrogen, carbon, nitrogen, oxygen, fluorine, iodine and chlorine such as ³H, ¹¹C, ¹⁴C, ¹⁸F, ¹²³I or ¹²⁵I. Compounds of the present disclosure and pharmaceutically acceptable salts of said compounds that contain the aforementioned isotopes and/or other isotopes of other atoms are within the scope of the present disclosure. Isotopically labelled compounds of the present disclosure, for example those into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

### Degradation Activity

Degradation may be determined by measuring the amount of a target protein in the presence of a bifunctional molecule as described herein and/or comparing this to the amount of the target protein observed in the absence of the bifunctional molecule. For example, the amount of target protein in a cell that has been contacted and/or treated with a bifunctional molecule as described herein may be determined. This amount may be compared to the amount of target protein in a cell that has not been contacted and/or treated with the bifunctional molecule (e.g. as a control). If the amount of target protein is decreased in the cell contacted and/or treated with the bifunctional molecule, the bifunctional molecule may be considered as facilitating and/or promoting the degradation and/or proteolysis of the target protein.

The amount of the target protein can be determined using methods known in the art, for example, by performing immunoblotting assays, Western blot analysis and/or ELISA with cells that have been contacted and/or treated with a bifunctional molecule.

Selective degradation and/or increased proteolysis may be considered to have occurred if at least a 10% decrease in the amount of a target protein is observed compared to the control, for example, 20%, 30%, 40%, 50%, 60%, 70%, 80%, 90% or 100% following administration of the bifunctional molecule to the cell.

For example, selective degradation and/or increased proteolysis may be considered to have occurred if at least a 10% decrease in the amount of a target protein is observed, (e.g. at least 20%, 30%, 40%, 50%, 60%, 70%, 80%, 90% or 100% decrease) within 4 hours or more (e.g. 4 hours, 8 hours, 12 hours, 24 hours, 30 hours, 36 hours, 42 hours, 48 hours, 54 hours, 60 hours, 66 hours and 72 hours) following administration of the bifunctional molecule to the cell. The bifunctional molecule may be administered at any concentration, e.g. a concentration between 0.01 nM to 10 μM, such as 0.01 nM, 0.1 nM, 1 nM, 10 nM, 100 nM, 1 M, and 10 μM. In some instances, an increase of at least 30%, 40%, 50%, 60%, 70%, 80%, 90%, or approximately 100% in the degradation of the target protein is observed following administration of the bifunctional molecule at a concentration of approximately 100 nM (e.g. following an incubation period of approximately 8 hours).

One measure of degrader activity of the bifunctional molecules is the DC₅₀ value. As used herein, DC₅₀ is the concentration required to reach 50% of the maximal degradation of the target protein. The bifunctional molecules described herein may comprise a DC₅₀ of less than or equal to 10000 nM, less than or equal to 1000 nM, less than or equal to 500 nM, less than or equal to 100 nM or less than or equal to 75 nM. In some cases, the bifunctional molecules comprise a DC₅₀ less than or equal to 50 nM, less than or equal to 25 nM, or less than or equal to 10 nM.

Another measure of the degrader activity of the bifunctional molecules is the Dₘₐₓ value. As used herein, Dₘₐₓ represents the maximal percentage of target protein degradation. The bifunctional molecules described herein may comprise a Dₘₐₓ of at least 50%, at least 60%, at least 70%, at least 80%, at least 90%, at least 95% or about 100%.

Yet another measure of the efficacy of the described bifunctional molecules may be their effect on cell viability and/or their IC₅₀ value. For example, an anti-proliferative effect of a bifunctional molecule as described herein may be assessed in a cell viability assay to provide an IC₅₀ value. As used herein, the IC₅₀ value represents the concentration at which 50% cell viability was observed in the cell viability assay (following administration of a bifunctional molecule as described herein). In terms of cell viability, the bifunctional molecules described herein may comprise an IC₅₀ of less than 1000 nM, less than 500 nM, less than 100 nM, less than 50 nM, less than 25 nM, less than 20 nM, or less than 10 nM. In some cases, the bifunctional molecules described herein may comprise an IC50 value of less than 5 nM.

### Pharmaceutical Compositions

The present disclosure provides a pharmaceutical composition comprising the bifunctional molecules described herein. In such compositions, the bifunctional molecule may be suitably formulated such that it can be introduced into the environment of the cell by a means that allows for a sufficient portion of the molecule to enter the cell to induce degradation of the target protein.

Accordingly, there is provided a pharmaceutical composition comprising a bifunctional molecule as described herein together with a pharmaceutically acceptable carrier.

Pharmaceutically acceptable carriers are well known to those skilled in the art and include, but are not limited to, phosphate buffer solutions and/or saline. Pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

In addition to the aforementioned carrier ingredients the pharmaceutical compositions described above may alternatively or additionally include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutical compositions may be present in any formulation typical for the administration of a pharmaceutical compound to a subject. Representative examples of typical formulations include, but are not limited to, capsules, granules, tablets, powders, lozenges, suppositories, pessaries, nasal sprays, gels, creams, ointments, sterile aqueous preparations, sterile solutions, aerosols, implants etc.

A pharmaceutical composition is formulated to be compatible with its intended route of administration. Examples of routes of administration include parenteral, e.g., intravenous, intradermal, subcutaneous, oral, transdermal, topical, transmucosal, vaginal and rectal administration.

The pharmaceutical compositions may include those suitable for oral, parenteral (including subcutaneous, intradermal, intramuscular and intravenous), topical (including dermal, buccal and sublingual), rectal, nasal and pulmonary administration e.g., by inhalation. The composition may, where appropriate, be conveniently presented in discrete dosage units and may be prepared by any of the methods well known in the art of pharmacy. Methods typically include the step of bringing into association an active compound with liquid carriers or finely divided solid carriers or both and then, if necessary, shaping the product into the desired formulation.

Pharmaceutical compositions suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. The bifunctional molecules may also be formulated as dispersible granules, which may for example be suspended in water before administration, or sprinkled on food. The granules may be packaged, e.g., in a sachet. Compositions suitable for oral administration wherein the carrier is a liquid may be presented as a solution or a suspension in an aqueous or non-aqueous liquid, or as an oil-in-water liquid emulsion. Compositions for oral administration include controlled release dosage forms, e.g., tablets wherein an active compound is formulated in an appropriate release-controlling matrix, or is coated with a suitable release-controlling film.

Pharmaceutical compositions suitable for parenteral administration include sterile solutions or suspensions of an active compound in aqueous or oleaginous vehicles. Injectable preparations may be adapted for bolus injection or continuous infusion. Such preparations are conveniently presented in unit dose or multi-dose containers, which are sealed after introduction of the formulation until required for use. Alternatively, the bifunctional molecule may be in powder form, which is constituted with a suitable vehicle, such as sterile, pyrogen-free water, before use.

The pharmaceutical composition may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins.

Pharmaceutical compositions suitable for topical formulation may be provided for example as gels, creams or ointments.

The bifunctional molecules described herein may be present in the pharmaceutical compositions as a pharmaceutically and/or physiologically acceptable salt, solvate or derivative.

As used herein, the term “pharmaceutically acceptable salt” refers to those salts, which are generally considered suitable for use in medicine (including in a veterinary context). For example, pharmaceutically acceptable salts may be those which can be contacted with the tissues of a mammalian subject (e.g. humans) without undue toxicity, irritation, allergic response or the like. By way of further example of suitable pharmaceutically acceptable salts, S. M. Berge et al., describe pharmaceutically acceptable salts in detail in J. Pharmaceutical Sciences, 1977, 66, 1-19, the entire contents of which are incorporated herein by reference.

Representative examples of pharmaceutically and/or physiologically acceptable salts of the bifunctional molecules of the disclosure may include, but are not limited to, acid addition salts formed with organic carboxylic acids such as acetic, lactic, tartaric, maleic, citric, pyruvic, oxalic, malonic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, hydrobromic, sulfuric, perchloric, phosphoric and sulfamic acids. Other pharmaceutically acceptable salts include (but are not limited to) adipate, alginate, ascorbate, aspartate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, 2-naphthalenesulfonate, nicotinate, nitrate, oleate, oxalate, palmitate, pamoate, pectinate, persulfate, 3-phenylpropionate, pivalate, propionate, stearate, thiocyanate, undecanoate, valerate salts, and the like.

In some examples, salts that may be derived from appropriate bases include alkali metal, alkaline earth metal, ammonium and N⁺(C₁₋₄alkyl)₄ salts. Representative alkali or alkaline earth metal salts include, but are not limited to, sodium, lithium, potassium, calcium, magnesium, and the like. Further pharmaceutically acceptable salts may include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Pharmaceutically and/or physiologically functional derivatives of compounds of the present invention are derivatives, which may be converted in the body into the parent compound. Such pharmaceutically and/or physiologically functional derivatives may also be referred to as “pro-drugs” or “bioprecursors”. Pharmaceutically and/or physiologically functional derivatives of compounds of the present disclosure may include hydrolysable esters or amides, particularly esters, in vivo.

It may be convenient or desirable to prepare, purify, and/or handle a corresponding pharmaceutically and/or physiologically acceptable solvate of the bifunctional molecules described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

### Uses of Moiety Z

As described herein, the moiety Z may form part of a bifunctional molecule intended for use in a method of targeted protein degradation, wherein the moiety Z acts to modulate, facilitate and/or promote proteasomal degradation of the target protein.

As such, according to a further aspect of the disclosure, there is provided a use of the moiety Z or a compound comprising moiety Z as described herein (e.g. as defined in any one of formula (I) to (V)) in a method of targeted protein degradation (e.g. an in vitro or in vivo method of targeted protein degradation). For example, moiety Z may find particular application as a promoter or facilitator of targeted protein degradation.

There is also provided a use of moiety Z or a compound comprising moiety Z (e.g. as defined in any one of formula (I) to (V)) in the manufacture of a bifunctional molecule suitable for targeted protein degradation.

### Therapeutic Methods and Uses

The bifunctional molecules of the present disclosure may modulate, facilitate and/or promote proteasomal degradation of a target protein. As such, there is provided a method of selectively degrading and/or increasing proteolysis of a target protein in a cell, the method comprising contacting and/or treating the cell with a bifunctional molecule as described herein. The method may be carried out in vivo or in vitro.

In particular, there is provided a method of selectively degrading and/or increasing proteolysis of a target protein in a subject in need thereof, the method comprising administering to the subject a therapeutically effective amount of a bifunctional molecule of the present disclosure.

As such, the bifunctional molecules of the present disclosure may find application in medicine and/or therapy. Specifically, the bifunctional molecules of the present disclosure may find use in the treatment and/or prevention of any disease or condition, which is modulated through the target protein. For example, the bifunctional molecules of the present disclosure may be useful in the treatment of any disease, which is modulated through the target protein by lowering the level of that protein in the cell, e.g. cell of a subject. Reduction of target protein levels in a cell following administration of a degrader of the present invention wherein activity of the selected protein is implicated in a disease state or a disorder, then it is to be understood that the degrader is useful in the treatment of that disease.

There is further provided the use of the bifunctional molecules as described herein in the manufacture of a medicament for the treatment and/or prevention of any disease or condition, which is modulated through the target protein. Additionally, there is provided the use of a moiety Z (e.g. as defined in any one of formulae (I) to (V) in the manufacture of a medicament for the treatment and/or prevention of any disease or condition, which is modulated through the target protein.

Diseases and/or conditions that may be treated and/or prevented by the molecules of the disclosure include any disease, which is associated with and/or is caused by an abnormal level of protein activity.

Such diseases and conditions include those whose pathology is related at least in part to an abnormal (e.g. elevated) level of a protein and/or the overexpression of a protein. For example, the bifunctional molecules may find use in the treatment and/or prevention of diseases where an elevated level of a protein is observed in a subject suffering from the disease. In other examples, the diseases and/or conditions may be those whose pathology is related at least in part to inappropriate protein expression (e.g., expression at the wrong time and/or in the wrong cell), excessive protein expression or expression of a mutant protein. In one example, a mutant protein disease is caused when a mutant protein interferes with the normal biological activity of a cell, tissue, or organ.

Accordingly, there is provided a method of treating and/or preventing a disease or condition, which is associated with and/or is caused by an abnormal level of protein activity, which comprises administering a therapeutically effective amount of a bifunctional compound as described herein.

Representative examples of the diseases and/or conditions that may be treated and/or prevented by the use of the described bifunctional compounds include (but are not limited to) cancer, asthma, multiple sclerosis, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease, (PKDI) or 4 (PKD2) Prader-Willi syndrome, Sickle-cell disease, Tay-Sachs disease, and Turner syndrome.

Further examples include, Alzheimer's disease, Amyotrophic lateral sclerosis (Lou Gehrig's disease), Anorexia nervosa, Anxiety disorder, Atherosclerosis, Attention deficit hyperactivity disorder, Autism, Bipolar disorder, Chronic fatigue syndrome, Chronic obstructive pulmonary disease, Crohn's disease, Coronary heart disease, Dementia, Depression, Diabetes mellitus type 1, Diabetes mellitus type 2, Epilepsy, Guillain-Barre syndrome, Irritable bowel syndrome, Lupus, Metabolic syndrome, Multiple sclerosis, Myocardial infarction, Obesity, Obsessive-compulsive disorder, Panic disorder, Parkinson's disease, Psoriasis, Rheumatoid arthritis, Sarcoidosis, Schizophrenia, Stroke, Thromboangiitis obliterans, Tourette syndrome, and Vasculitis.

Yet further examples include aceruloplasminemia, Achondrogenesis type II, achondroplasia, Acrocephaly, Gaucher disease type 2, acute intermittent porphyria, Canavan disease, Adenomatous Polyposis Coli, ALA dehydratase deficiency, adenylosuccinate lyase deficiency, Adrenogenital syndrome, Adrenoleukodystrophy, ALA-D porphyria, ALA dehydratase deficiency, Alkaptonuria, Alexander disease, Alkaptonuric ochronosis, alpha 1-antitrypsin deficiency, alpha-1 proteinase inhibitor, emphysema, amyotrophic lateral sclerosis, Alstrom syndrome, Alexander disease, Amelogenesis imperfecta, ALA dehydratase deficiency, Anderson-Fabry disease, androgen insensitivity syndrome, Anemia, Angiokeratoma Corporis Diffusum, Angiomatosis retinae (von Hippel-Lindau disease), Apert syndrome, Arachnodactyly (Marfan syndrome), Stickler syndrome, Arthrochalasis multiplex congenital (Ehlers-Danlos syndrome arthrochalasia type), ataxia telangiectasia, Rett syndrome, primary pulmonary hypertension, Sandhoff disease, neurofibromatosis type II, Beare-Stevenson cutis gyrata syndrome, Mediterranean fever, familial, Benjamin syndrome, beta-thalassemia, Bilateral Acoustic Neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dube syndrome, Brittle bone disease (osteogenesis imperfecta), Broad Thumb-Hallux syndrome (Rubinstein-Taybi syndrome), Bronze Diabetes/Bronzed Cirrhosis (hemochromatosis), Bulbospinal muscular atrophy (Kennedy's disease), Burger-Grutz syndrome (lipoprotein lipase deficiency), CGD Chronic granulomatous disorder, Campomelic dysplasia, biotinidase deficiency, Cardiomyopathy (Noonan syndrome), Cri du chat, CAVD (congenital absence of the vas deferens), Caylor cardiofacial syndrome (CBAVD), CEP (congenital erythropoietic porphyria), cystic fibrosis, congenital hypothyroidism, Chondrodystrophy syndrome (achondroplasia), otospondylomegaepiphyseal dysplasia, Lesch-Nyhan syndrome, galactosemia, Ehlers-Danlos syndrome, Thanatophoric dysplasia, Coffin-Lowry syndrome, Cockayne syndrome, (familial adenomatous polyposis), Congenital erythropoietic porphyria, Congenital heart disease, Methemoglobinemia/Congenital methaemoglobinaemia, achondroplasia, X-linked sideroblastic anemia, Connective tissue disease, Conotruncal anomaly face syndrome, Cooley's Anemia (beta-thalassemia), Copper storage disease (Wilson's disease), Copper transport disease (Menkes disease), hereditary coproporphyria, Cowden syndrome, Craniofacial dysarthrosis (Crouzon syndrome), Creutzfeldt-Jakob disease (prion disease), Cockayne syndrome, Cowden syndrome, Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, Degenerative nerve diseases including de Grouchy syndrome and Dejerine-Sottas syndrome, developmental disabilities, distal spinal muscular atrophy, type V, androgen insensitivity syndrome, Diffuse Globoid Body Sclerosis (Krabbe disease), Di George's syndrome, Dihydrotestosterone receptor deficiency, androgen insensitivity syndrome, Down syndrome, Dwarfism, erythropoietic protoporphyria, Erythroid 5-aminolevulinate synthetase deficiency, Erythropoietic porphyria, erythropoietic protoporphyria, erythropoietic uroporphyria, Friedreich's ataxia, familial paroxysmal polyserositis, porphyria cutanea tarda, familial pressure sensitive neuropathy, primary pulmonary hypertension (PPH), Fibrocystic disease of the pancreas, fragile X syndrome, galactosemia, genetic brain disorders, Giant cell hepatitis (Neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), Hyperandrogenism, Hypochondroplasia, Hypochromic anemia, Immune system disorders, including X-linked severe combined immunodeficiency, Insley-Astley syndrome, Jackson-Weiss syndrome, Joubert syndrome, Lesch-Nyhan syndrome, Jackson-Weiss syndrome, Kidney diseases, including hyperoxaluria, Klinefelter's syndrome, Kniest dysplasia, Lacunar dementia, Langer-Saldino achondrogenesis, ataxia telangiectasia, Lynch syndrome, Lysyl-hydroxylase deficiency, Machado-Joseph disease, Metabolic disorders, including Kniest dysplasia, Marfan syndrome, Movement disorders, Mowat-Wilson syndrome, cystic fibrosis, Muenke syndrome, Multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, Polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford Progeria Syndrome), progressive chorea, chronic hereditary (Huntington) (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), Recurrent polyserositis, Retinal disorders, Retinoblastoma, Rett syndrome, RFALS type 3, Ricker syndrome, Riley-Day syndrome, Roussy-Levy syndrome, severe achondroplasia with developmental delay and acanthosis nigricans (S ADD AN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita), SEMD, Strudwick type (spondyloepimetaphyseal dysplasia, Strudwick type), Shprintzen syndrome, Skin pigmentation disorders, Smith-Lemli-Opitz syndrome, South-African genetic porphyria (variegate porphyria), infantile-onset ascending hereditary spastic paralysis, Speech and communication disorders, sphingolipidosis, Tay-Sachs disease, spinocerebellar ataxia, Stickler syndrome, stroke, androgen insensitivity syndrome, tetrahydrobiopterin deficiency, beta-thalassemia, Thyroid disease Tomaculous neuropathy (hereditary neuropathy with liability to pressure palsies) Treacher Collins syndrome, Triplo X syndrome (triple X syndrome), Trisomy 21 (Down syndrome), Trisomy X, VHL syndrome (von Hippel-Lindau disease), Vision impairment and blindness (Alstrom syndrome), Vrolik disease, Waardenburg syndrome, Warburg Sjo Fledelius Syndrome, Weissenbacher-Zweymüller syndrome, Wolf-Hirschhorn syndrome, Wolff Periodic disease, Weissenbacher-Zweymüller syndrome and Xeroderma pigmentosum. Representative examples of cancers that may be treated and/or prevented using the described bifunctional molecules include but, are not limited to squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; multiple myeloma, sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Further examples include, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

As used herein, the term “patient” or “subject” is used to describe an animal, such as a mammal (e.g. a human or a domesticated animal), to whom treatment, including prophylactic treatment, with the compositions according to the present disclosure is provided. For treatment of those infections, conditions or disease states which are specific to a specific animal such as a human patient, the term patient refers to that specific animal, including a domesticated animal such as a dog or cat or a farm animal such as a horse, cow, sheep, etc. In general, in the present invention, the term patient refers to a human patient unless otherwise stated or implied from the context of the use of the term.

### Assays

The disclosure also encompasses a method of screening bifunctional molecules to identify suitable target protein binding ligands and linkers for use in the bifunctional molecules described herein, e.g. a bifunctional molecule that is able to effectively modulate, facilitate and/or promote proteolysis of a target protein. This method may assist in identifying suitable linkers for a particular target protein binding partner such that the level of degradation is further optimised.

The method may comprise:
a. providing a bifunctional molecule comprising:
   (i) a first ligand comprising a structure according to Z (as defined in any of the formulae for Z disclosed herein);
   (ii) a second ligand that binds to a target protein (a target protein binding ligand); and
   (iii) a linker that covalently attaches the first and second ligands;
b. contacting a cell with the bifunctional molecule; and
C. detecting degradation of the target protein in the cell.

This method may further comprise the steps of:
d. detecting degradation of the target protein in the cell in the absence of the bifunctional molecule; and
e. comparing the level of degradation of the target protein in the cell contacted with the bifunctional molecule to the level of degradation of the target protein in the absence of the bifunctional molecule;
wherein an increased level of degradation of the target protein in the cell contacted with the bifunctional molecule indicates that the bifunctional molecule has facilitated and/or promoted the degradation of the target protein.

In such methods, a step of detecting degradation of the target protein may comprise detecting changes in levels of a target protein in a cell. For example, a reduction in the level of the target protein indicates degradation of the target protein. An increased reduction in the level of the target protein in the cell contacted with the bifunctional molecule (compared to any reduction in the levels of target protein observed in the cell in the absence of the bifunctional molecule) indicates that the bifunctional molecule has facilitated and/or promoted the degradation of the target protein.

The method may further comprise providing a plurality of linkers, each one being used to covalently attach the first and second ligands together to form a plurality of bifunctional molecules. The level of degradation provided by each one of the plurality of bifunctional molecules may be detected and compared. Those bifunctional molecules showing higher levels of target protein degradation indicate preferred and/or optimal linkers for use with the selected target protein binding partner.

The method may be carried out in vivo or in vitro.

### Compound Library

The disclosure also provides a library of bifunctional molecules, the library comprising a plurality of bifunctional molecules, the plurality of bifunctional molecules comprising a plurality of Z moieties covalently linked to a selected target protein binding partner.

As such, the target protein binding partner may be pre-selected and the Z moiety may not be determined in advance. The library may be used to determine the activity of a candidate Z moiety of a bifunctional molecule in modulating, promoting and/or facilitating selective protein degradation of a target protein.

The disclosure also includes a library of bifunctional molecules, the library comprising a plurality of bifunctional molecules, the plurality of bifunctional molecules comprising a plurality of target protein binding ligands and a selected Z moiety. As such, the Z moiety of the bifunctional molecule may be pre-selected and the target protein may not be determined in advance. The library may be used to determine the activity of a putative target protein binding ligand and its value as a binder of a target protein to facilitate target protein degradation.

### Methods of Manufacture

According to a further aspect of the disclosure, there is provided a method of making a bifunctional molecule as described herein.

The method of making the bifunctional molecule may comprise the steps of:
(a) providing a first ligand or moiety comprising a structure according to Z (as defined in any one of the formulae for Z disclosed herein);
(b) providing a second ligand or moiety that binds to a target protein (e.g. a target protein binding ligand as defined herein); and
(c) linking (e.g. covalently linking) the first and second ligands or moieties using a linker as defined herein.

In other examples, the method of making the bifunctional molecule may comprise the steps of:
(a) providing a target protein binding ligand (as defined herein);
(b) linking (e.g. covalently linking) a linker (as defined herein) to the target protein binding ligand to provide a target protein binding ligand-linker conjugate (TBL-L);
(c) further reacting the linker moiety of the conjugate to add and/or form a structure according to Z (as defined in any of formulae (I) to (III)) thereon to provide the bifunctional molecule having the general formula TBL-L-Z.

It should be understood that throughout this specification, the terms “comprise”, “comprising” and/or “comprises” is/are used to denote that aspects, embodiments and examples of this disclosure “comprise” a particular feature or features. It should be understood that this/these terms may also encompass aspects, embodiments and/or examples which “consist essentially of” or “consist of” the relevant feature or features.

### Disclaimer

The bifunctional molecule of the present invention may not comprise one or more structures. In examples, the bifunctional molecule of the present invention does not comprise bifunctional molecules, or Z, having the structure of the disclaimer defined below.

### Aryl/Heteroaryl Cyanoacrylamides

In examples of the present invention, the bifunctional molecule of the first aspect does not comprise bifunctional molecules, or Z, having the structure of Disclaimer 2, as defined below.

In embodiments, the bifunctional molecule of the first aspect does not comprise a bifunctional molecule, or Z, having the general formula (DII):

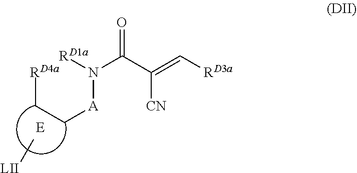

wherein
R^{D1a} is selected from C₁ to C₆ alkyl, benzyl, substituted benzyl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclic or heterocyclic group;
A is absent or is CR^{D2a}R^{D2a}′.
ring E is selected from aryl, heteroaryl, substituted aryl and substituted heteroaryl;
R^{D2a} and R^{D2a}′ are each independently selected from H and C₁ to C₆ alkyl, optionally wherein the C₁ to C₆ alkyl is substituted with one or more heteroatoms selected from N, O or S, or wherein R² and R²′ together form a 3-, 4-, 5- or 6-membered carbocyclic or heterocyclic ring;
R^{D3a} is selected from C₁ to C₆ alkyl, aryl, heteroaryl, substituted aryl, substituted heteroaryl, carbocyclyl, substituted carbocyclyl, heterocyclyl and substituted heterocyclyl, optionally wherein the C₁ to C₆ alkyl is substituted with one or more heteroatoms selected from halo, N, O and S and/or is substituted with a carbocyclic or heterocyclic group;
R^{D4a} is H, C₁ to C₆ alkyl, optionally wherein the C₁ to C₆ alkyl is substituted with one or more heteroatoms selected from N, O or S;
or wherein R^{D1a} and R^{D}4a together form a 5-, 6-, or 7-membered heterocyclic ring;
or wherein when A is CR^{D2a}R^{D2a}″:
R^{D1a} and R^{D2a} together form a 5-, 6-, or 7-membered heterocyclic ring; or
R^{D2a} and R^{D4a} together form a 5-, 6-, or 7-membered heterocyclic or carbocyclic ring; and
LII shows the point of attachment of the linker.

In examples of Disclaimer 2, on ring E, groups R^{D4a} and A may be held at adjacent positions on the aryl, heteroaryl, substituted aryl or substituted heteroaryl ring. In other words, the R^{D4a} and A groups may be in a 1,2-substitution pattern with one another, or may be separated by 3 bonds. For the avoidance of doubt, where ring E is a heteroaryl or substituted heteroaryl, a heteroatom contained within ring E may be directly bonded to A or R^{D4a}.

As shown in formula (DII) above, and in examples of Disclaimer 2, the linker is appended to moiety Z via ring E. The linker may be attached to moiety Z by way of a covalent bond between an atom on the linker and an atom contained in the ring system of the optionally substituted aryl or heteroaryl group of ring E. This linker may be attached to ring E at any position on the optionally substituted aromatic or heteroaromatic ring (provided it has the correct valency and/or is chemically suitable). For example, the linker may replace a hydrogen atom at any position on the aromatic or heteroaromatic ring.

In other examples of Disclaimer 2, the bifunctional molecule, or Z, to be disclaimed may comprise a structure as shown in formula (DII) above, wherein:
A, ring E, X and R^{D4a} are as defined above; and wherein
R^{D1a} is selected from optionally substituted C₁ to C₆ alkyl, optionally substituted C₁ to C₆ haloalkyl, optionally substituted benzyl, optionally substituted carbocyclyl, and optionally substituted heterocyclyl;
R^{D2a} and R^{D2a}′ are each independently selected from H and optionally substituted C₁ to C₆ alkyl, or wherein R^{D2a} and R^{D3a}′ together form a 3-, 4-, 5- or 6-membered optionally substituted carbocyclic or heterocyclic ring; and
R^{D3a} is selected from optionally substituted C₁ to C₆ alkyl, optionally substituted aryl, optionally substituted heteroaryl, optionally substituted carbocyclyl and optionally substituted heterocyclyl.

In further examples of Disclaimer 2, in those cases where R^{D1a} and R^{D4a} together form a 5-, 6-, or 7-membered heterocyclic ring, Z to be disclaimed may be represented by formula (DIIa):

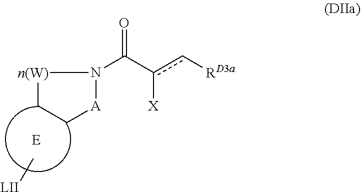

wherein A, B, R³, X and LII are as defined for formula (DII); and
n is 1, 2 or 3;
W is selected from CR^{W1}R^{W2}, O, NR^{W3}, and S; and
R^{W1}R^{W2} and R^{W3} are each independently selected from H and C₁ to C₆ alkyl; and
wherein when n is 2 or 3, each W is independently selected from CR^{W1}R^{W2}, O, NR^{W3}, and S.

In further examples of Disclaimer 2, in those cases, where R^{D1a} and R^{D2a} together form a 5-, 6-, or 7-membered heterocyclic ring, the bifunctional molecule, or Z to be disclaimed may be represented as formula (DIIb):

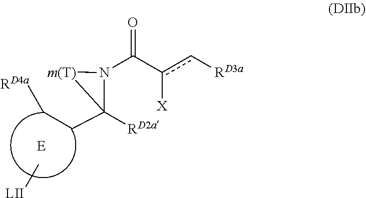

wherein ring E, R^{D2a}′, R^{D3a}, R^{D4a}, X and LII are as defined for formula (DII);
m is 3, 4 or 5;
each T is independently selected from CR^{T1}R^{T2}, O, NR^{T3}, and S; and
R^{T1}, R^{T2} and R^{T3} are each independently selected from H and C₁ to C₆ alkyl.

In further examples of Disclaimer 2, in those cases where R^{D2a} and R^{D4a} together form a 5-, 6-, or 7-membered heterocyclic or carbocyclic ring, Z may be represented as formula (DIIc):

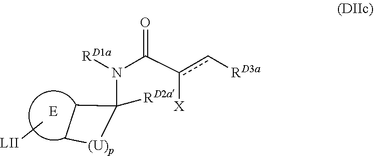

wherein ring E, R^{D1a}, R^{D2a}′, R^{D3a}, X and LII are as defined for formula (I);
p is 2, 3 or 4; and
each U is independently selected from CR^{U1}R^{U2}, O, NR^{U3}, and S; and
R^{U1}, R^{U2} and R^{U3} are each independently selected from H and C₁ to C₆ alkyl.

In some examples of Disclaimer 2, the bifunctional molecule, or Z, to be disclaimed may comprise a structure according to formula (ZII):

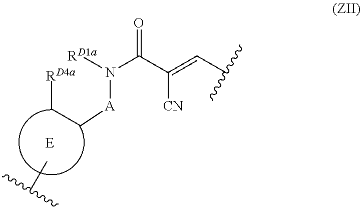

wherein
R^{D1a} is not H;
A is absent or is CR^{D2a}R^{D2a}′.
ring E is selected from aryl, heteroaryl, substituted aryl and substituted heteroaryl;
wherein R^{D1a} and R^{D4a} are not joined to form a ring; or
wherein R^{D1a} and R^{D4a} together form a 5-, 6-, or 7-membered heterocyclic ring; or
wherein when A is CR^{D2a}R^{D2a}″:
R^{D1a} and R^{D2a} together form a 5-, 6-, or 7-membered heterocyclic ring; or
R^{D2a} and R^{D4a} together form a 5-, 6-, or 7-membered heterocyclic or carbocyclic ring.

In further examples of Disclaimer 2, the linker (LII) present on the structure to be disclaimed has the structure:

(LIIₓ)_{qa}
wherein each LIIx represents a subunit of LII that is independently selected from CR^{L1a}R^{L2a}, O, C═O, S, SO, SO₂, NR^{L3a}, SONR^{L4a}, SONR^{L5a}C═O, CONR^{L6a}, NR^{L7a}CO, C(R^{L8a})═C(R^{L9a}), C≡C, aryl, substituted aryl, heteroaryl, substituted heteroaryl, cycloalkyl, substituted cycloalkyl, heterocycloalkyl and substituted heterocycloalkyl groups;
wherein R^{L1a}, R^{L2a}, R^{L3a}, R^{L4a}, R^{L5a}, R^{L6a}, R^{L7a}, R^{L8a} and R^{L9a} are each independently selected from H, halo, C₁ to C₆ alkyl, C₁ to C₆, haloalkyl, —OH, —O(C₁ to C₆ alkyl), —NH₂, —NH(C₁ to C₆ alkyl), —NO₂, —CN, —CONH₂, —CONH(C₁ to C₆ alkyl), —CON(C₁ to C₆ alkyl)₂, —SO₂(C₁ to C₆ alkyl), —CO₂(C₁ to C₆ alkyl), and —CO(C₁ to C₆ alkyl); and
qa is an integer between 1 and 30.

In examples of Disclaimer 2, the linker (LII) present on the structure to be disclaimed the linker is or comprises one or more of:

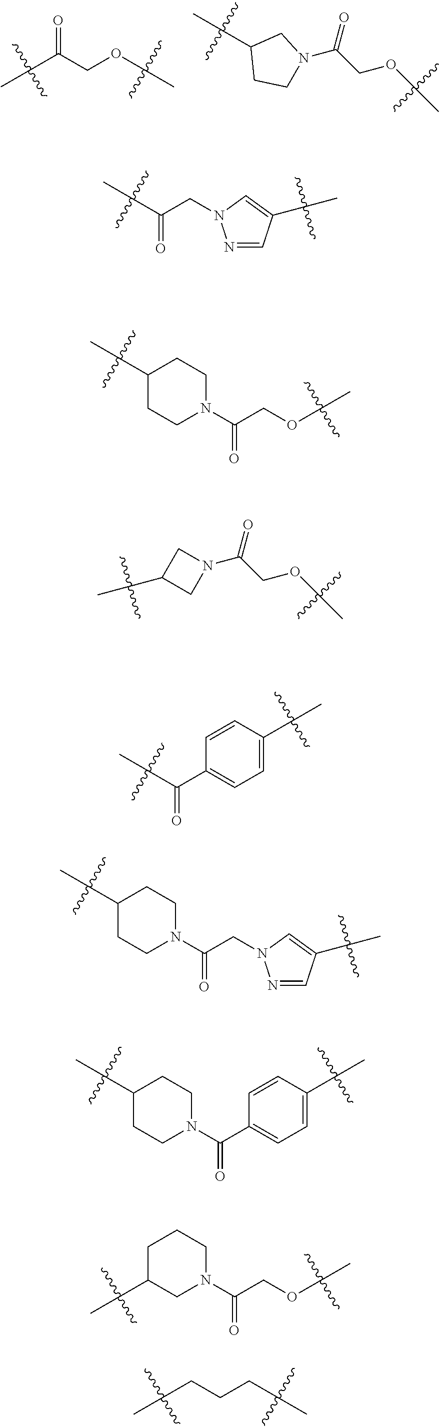

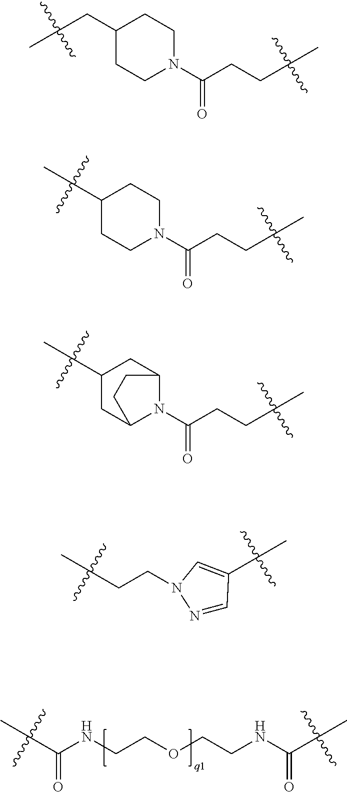

wherein q1 is any integer between 1 and 20, or between 1 and 10 (e.g. between 1 and 5).

Alternatively, in any of the examples of Disclaimer 2, the linker is or comprises one or more of:

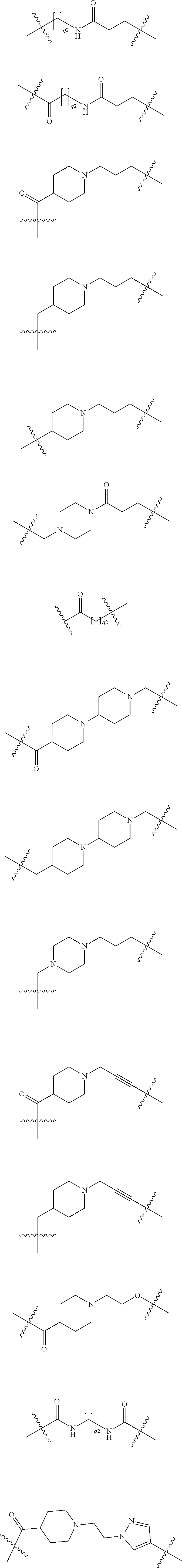

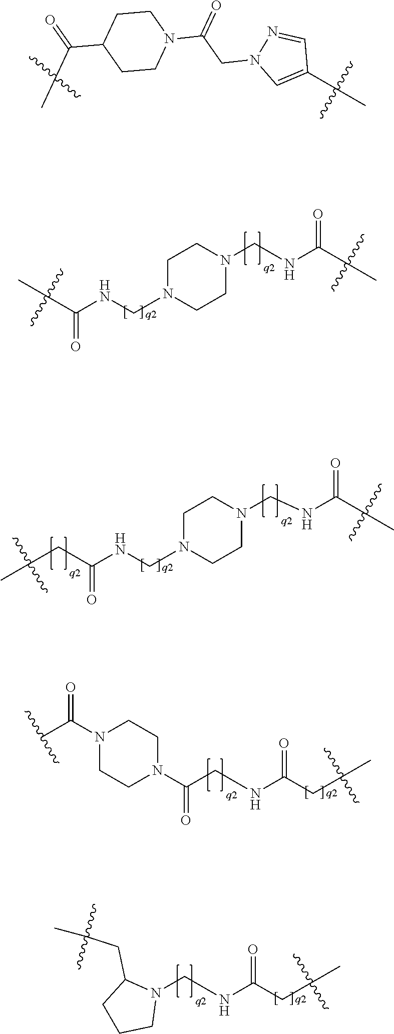

wherein q2 is any integer between 1 and 20, or between 1 and 10 (e.g. 3, 4, 6 or 10).

In some cases, the structures shown above represent the entire linker. In other examples, the linker of the bifunctional molecule to be disclaimed may comprise a plurality of the structures shown above.

In these structures, the wavy lines are shown over the bond(s) that forms the link with further portions of the linker, the TBL and Z moieties respectively.

### Definitions

In the discussion above, reference is made to a number of terms, which are to be understood to have the meanings provided below, unless a context indicates to the contrary. The nomenclature used herein for defining compounds, in particular the compounds described herein, is intended to be in accordance with the rules of the International Union of Pure and Applied Chemistry (IUPAC) for chemical compounds, specifically the “IUPAC Compendium of Chemical Terminology (Gold Book)” (see A. D. Jenkins et al., Pure & Appl. Chem., 68, 2287-2311 (1996)). For the avoidance of doubt, if an IUPAC rule is contrary to a definition provided herein, the definition herein is to prevail.

As used herein, the term “aryl” refers to a mono- or polycyclic aromatic hydrocarbon system having 6 to 14 carbon atoms, in some cases having 6 to 10 carbon atoms. Representative examples of suitable “aryl” groups include, but are not limited to, phenyl, biphenyl, naphthyl, 1-naphthyl, 2-naphthyl and anthracenyl. As used herein, “substituted aryl” refers to an aryl group as defined herein which comprises one or more substituents on the aromatic ring. When an aryl group is substituted, any hydrogen atom(s) may be replaced with the substituent(s), providing valencies are satisfied.

As used herein, “heteroaryl” may be a single or fused ring system having one or more aromatic rings containing 1 or more, in some cases 1 to 3, in some cases 1 to 2, in some cases a single O, N and/or S heteroatom(s). The term “heteroaryl” may refer to a mono- or polycyclic heteroaromatic system having 5 to 10 ring atoms. Representative examples of heteroaryl groups may include, but are not limited to, pyrrolyl, furanyl, thiophenyl, pyrazolyl, imidazolyl, oxazolyl, isoxazolyl, thiazolyl, pyridinyl, pyrimidinyl, pyridazinyl, pyrazinyl, indolyl, benzofuranyl, benzothiazolyl, benzimidazolyl, indazolyl, benzoxazolyl, benzisoxazolyl etc. As used herein, “substituted heteroaryl” refers to a heteroaryl group as defined herein which comprises one or more substituents on the heteroaromatic ring.

As used herein, the term “alkyl” refers to a straight or branched chain hydrocarbyl group. The chain may be saturated or unsaturated, e.g. in some cases the chain may contain one or more double or triple bonds.

As used herein, “C₁-C₆ alkyl” refers to a straight or branched chain hydrocarbyl group containing from 1 to 6 carbon atoms. As used herein, a “C₁-C₃ alkyl” refers to a straight or branched chain hydrocarbyl group containing from 1 to 3 carbon atoms. Representative examples are methyl, ethyl, n-propyl, iso-propyl, n-butyl, sec-butyl, iso-butyl, tert-butyl, n-pentyl, isopentyl, neopentyl, n-hexyl, isohexyl, neohexyl, etc. When an alkyl group is substituted, any hydrogen atom(s), CH₃, CH₂ or CH group(s) may be replaced with the substituent(s), providing valencies are satisfied.

As used herein, a “cycloalkyl” is a ring containing 3 to 10 carbon atoms, in some cases 3 to 8, or in some cases 5 to 6 carbon atoms. The ring may be saturated or unsaturated, e.g. in some cases the ring may contain one or more double or triple bonds. As used herein, a C₃-C₇ cycloalkyl is a cycloalkyl containing 3 to 7 carbon atoms in the ring. As used herein, a C₃-C₆ cycloalkyl is a cycloalkyl containing 3 to 6 carbon atoms in the ring.

Representative examples of cycloalkyl groups include cyclopropyl, cyclobutyl, cyclopentyl, cyclohexyl, cycloheptyl, cyclooctyl, cyclobutenyl, cyclopentenyl, cyclohexenyl, cycloheptenyl, cyclooctenyl, cyclooctynl etc. As used herein, “substituted cycloalkyl” refers to a cycloalkyl group as defined herein which comprises one or more substituents on the cycloalkyl ring. When a cycloalkyl group is substituted, any hydrogen atom(s) may be replaced with the substituent(s), providing valencies are satisfied.

The term “alkenyl” defines monovalent groups derived from alkenes by removal of a hydrogen atom from any carbon atom, wherein the term “alkene” is intended to define acyclic branched or unbranched hydrocarbons having the general formula CnH₂ₙ, wherein n is an integer ≥2. Examples of alkenyl groups include ethenyl, n-propylenyl, iso-propylenyl, n-butylenyl, sec-butylenyl, iso-butylenyl and tert-butylenyl. When an alkenyl group is substituted, any hydrogen atom(s) may be replaced with the substituent(s), providing valencies are satisfied. Where the alkenyl comprises a divalent hydrocarbon radical, this moiety may sometimes be referred to herein as an alkenylene.

The term “alkynyl” defines monovalent groups derived from alkynes by removal of a hydrogen atom from any carbon atom, wherein the term “alkyne” is intended to define acyclic branched or unbranched hydrocarbons having the general formula CnH₂ₙ₋₂, wherein n is an integer ≥2. Examples of alkynyl groups include ethynyl, n-propylynyl, iso-propylynyl, n-butylynyl, sec-butylynyl, iso-butylynyl and tert-butylynyl. When an alkynyl group is substituted, any hydrogen atom(s) may be replaced with the substituent(s), providing valencies are satisfied. Where the alkynyl comprises a divalent hydrocarbon radical, this moiety may sometimes be referred to herein as an alkynylene.

“Benzyl” as used herein refers to a —CH₂Ph group. As used herein, a “substituted benzyl” refers to a benzyl group as defined herein which comprises one or more substituents on the aromatic ring. When a benzyl group is substituted, any hydrogen atom(s) may be replaced with the substituent(s), providing valencies are satisfied.

As used herein, “heterocycloalkyl” refers to a monocyclic or polycyclic ring having in one or more rings of the ring system at least one heteroatom selected from O, N and S (e.g. from one to five ring heteroatoms independently selected from the group consisting of O, N and S). The one or more rings may also contain one or more double bonds provided that the one or more rings are not fully aromaticized. The one or more rings of the heterocycloalkyl may comprise 3 to 10 atoms, in some cases 3 to 8 atoms. The one or more rings may be aliphatic. The one or more rings may be saturated or unsaturated, e.g. in some cases the one or more rings may contain one or more double or triple bonds. Any N heteroatom present in the heterocycloalkyl group may be C₁ to C₆ alkyl-substituted. In some cases, the heterocycloalkyl is a monocyclic or bicyclic ring, such as a monocyclic ring. Representative examples of heterocycloalkyl groups include, but are not limited to, pyrrolidinyl, tetrahydrofuranyl, dioxolanyl, dithiolanyl, thiazolidinyl, isothiazolidinyl, oxazolidinyl, isoxazolidinyl, pyrazolidinyl, imidazolidinyl, piperidinyl, piperazinyl, N-alkylpiperazinyl, morpholinyl, dioxanyl, oxazolidinyl, tetrahydropyranyl, diazaspiroundecane, diazaspiroheptane, azaspiroheptane, diazaspirodecane, octahydropyrrolopyrrole, etc. As used herein, “substituted heterocycloalkyl” refers to a heterocycloalkyl group as defined herein which comprises one or more substituents on the heterocycloalkyl ring.

As used herein, —CH(aryl)-, —CH(substituted aryl)-, —CH(heteroaryl)- and —CH(substituted heteroaryl) refers to a methylene moiety that comprises an aryl, substituted aryl, heteroaryl or substituted heteroarylsubstituent and is the attachment point for the linker L.

As used herein, the term “heterocyclyl” refers to a monovalent radical derived from a heterocycle. A heterocycle is a cyclic compound (a compound comprising one or more rings of connected atoms) having as ring members atoms of at least two different elements (such as carbon and nitrogen).

As used herein, a “carbocyclic ring” is a ring containing 3 to 10 carbon atoms, in some cases 3 to 8 carbon atoms, or in some cases 5 to 6 carbon atoms. The ring may be aliphatic. Thus, as used herein, references to “carbocyclyl” and “substituted carbocyclyl” groups may refer to aliphatic carbocyclyl groups and aliphatic substituted carbocyclyl groups. The ring may be saturated or unsaturated, e.g. in some cases the ring may contain one or more double or triple bonds. Representative examples of carbocyclyl groups include cyclopropyl, cyclobutyl, cyclopentyl, cyclohexyl, cycloheptyl, cyclooctyl, cyclobutenyl, cyclopentenyl, cyclohexenyl, cycloheptenyl, cyclooctenyl, cyclooctynl etc. As used herein, “substituted carbocyclyl” refers to a carbocyclyl group as defined herein which comprises one or more substituents on the carbocyclic ring. When a carbocyclyl group is substituted, any hydrogen atom(s) may be replaced with the substituent(s), providing valencies are satisfied.

As used herein, a “heterocyclic ring” (or heterocyclyl) may comprise at least 1 heteroatom selected from O, N and S. The heterocyclic ring may be a monocyclic or polycyclic ring, each ring comprising 3 to 10 atoms, in some cases 3 to 8 atoms. The one or more rings may be aliphatic. Thus, as used herein, references to “heterocyclyl” and “substituted heterocyclyl” groups may refer to aliphatic heterocyclyl groups and aliphatic substituted heterocyclyl groups. The one or more rings may be saturated or unsaturated, e.g. in some cases the one or more rings may contain one or more double or triple bonds. Any N heteroatom present in the heterocyclic group may be C₁ to C₆ alkyl-substituted. In some cases, the heterocyclyl is a monocyclic or bicyclic ring, such as a monocyclic ring. In other examples, the heterocyclyl may be a bicyclic ring, which may, in some cases be a fused ring. Representative examples of heterocyclyl groups include, but are not limited to, pyrrolidinyl, tetrahydrofuranyl, dioxolanyl, dithiolanyl, thiazolidinyl, isothiazolidinyl, oxazolidinyl, isoxazolidinyl, pyrazolidinyl, imidazolidinyl, piperidinyl, piperazinyl, N-alkylpiperazinyl, morpholinyl, dioxanyl, oxazolidinyl, tetrahydropyranyl, diazaspiroundecane, diazaspiroheptane, azaspiroheptane, diazaspirodecane, octahydropyrrolopyrrole, pyrrolizidinyl, etc. As used herein, “substituted heterocyclyl” refers to a heterocyclyl group as defined herein which comprises one or more substituents on the heterocyclic ring.

As used herein, where a group comprising carbon atoms is defined as “saturated”, only single bonds bind the carbon atoms to one another. Where a group comprising carbon atoms is defined as “unsaturated”, at least two of the carbon atoms are connected by a double or triple bond. For the avoidance of doubt, unsaturated compounds may comprise any number of double and/or triple bonds, provided that

“Cycloalkene” is used herein to refer to an unsaturated monocyclic hydrocarbon having one endocyclic double bond.

The term “spiro” is used to refer to moieties comprising two or more ring systems, wherein at least two of the ring systems are connected by just one atom (typically a quaternary carbon atom).

“Monocyclic” is used herein to refer to moieties comprising one ring of atoms. “Bicyclic” is used herein to refer to moietes that feature two joined rings of atoms. “Tricyclic” is used herein to refer to moieties that feature three joined rings of atoms. “Polycyclic” is used herein to refer to moieties that comprise two or more joined rings. Unless the context indicates otherwise, bicyclic and polycyclic systems may comprise a fused ring system (in which at least two rings share a common bond). In other examples, the two or more rings may be joined by a bond between atoms on each of the two or more rings. In other examples, the bicyclic system may comprise a spiro centre (as defined above).

The term “bridged” is used herein to refer to a cyclic compound, or ring, comprising two bridgehead atoms (typically two carbon atoms of the cyclic compound or ring) that are connected by one or more atoms lying outside of the ring (such as one to three atoms lying outside of the ring). Bridged rings comprise two rings sharing three or more atoms. In some examples, the bridgehead atoms are separated within the ring by at least one carbon atom. In some examples, a ring may be bridged by between 1 and 3 bridging atoms which lie outside of the ring to form a bridging group (optionally wherein the bridging atoms are selected from C, N, O and S). As used herein, a “C₁₋₃ bridge” is a bridging group comprising between 1 and 3 carbon bridging atoms. The bridging group may compirise one to three atoms lying outside of the ring, of which one, two or three of those atoms are carbon. In some cases, the bridging group may additionally comprise non-carbon atoms (such as a heteroatom selected from N, O and S). By way of example, as used herein, a “C₁₋₃ bridge” may refer to a bridging group comprising between 1 and 3 atoms of which one, two or three are carbon and the remainder (if any) are selected from N, O and S. The bridging group may be a C₁ to C₃ alkylene (such as methylene, ethylene or propylene). The C₁ to C₃ alkylene bridging group may be optionally substituted with any suitable substituent as described herein. For example, C₁ to C₃ alkylene bridging group may be optionally substituted with one or two substituents each independently selected from the group consisting of halo, C₁ to C₃ alkyl, C₁ to C₃ haloalkyl and C₁ to C₃ alkoxy.

The term “fused” is used to refer to moieties comprising two or more ring systems, wherein at least two of the ring systems are connected by a [1,2] ring junction, i.e. a moiety comprising two or more ring systems wherein two, or more, of the rings present share a bond in each respective ring structure.

The term “aliphatic” refers to acyclic or cyclic, saturated or unsaturated compounds, excluding aromatic compounds, where “aromatic” defines a cyclically conjugated molecular entity with a stability (due to delocalisation) significantly greater than that of a hypothetical localised structure. The Huckel rule is often used in the art to assess aromatic character; monocyclic planar (or almost planar) systems of trigonally (or sometimes digonally) hybridised atoms that contain (4n+2) π-electrons (where n is a non-negative integer) will exhibit aromatic character. The rule is generally limited to n=0 to 5.

The term “hydrocarbyl” refers to a monovalent radical derived from a hydrocarbon by the removal of a hydrogen atom from the hydrocarbon. A hydrocarbon is any molecule comprising only the elements carbon and hydrogen. Hydrocarbons may be aliphatic, aromatic, unsaturated or saturated.

As used herein, an alkoxy refers to an alkyl group, as defined above, appended to the parent molecular moiety through an oxy group, —O—. As used herein, a C₁₋₄alkoxy refers to a C₁₋₄ alkyl group (as defined above), appended to the parent molecular moiety through a oxy group, —O—. Representative examples of alkoxy include, but are not limited to, methoxy, ethoxy, propoxy, 2-propoxy, butoxy, tert-butoxy, pentyloxy, hexyloxy etc.

As used herein, “alkoxyalkyl” may refer to a moiety derived from an alkyl moiety in which a hydrogen atom at any position of the alkyl is substituted with an alkoxy moiety. Examples of alkoxyalkyl groups include methoxyethyl, methoxypropyl, ethoxymethyl and the like.

As used herein, the term “alkylcarbonyl” refers to carbonyl having alkyl mentioned above. Examples of alkylcarbonyl include C₁₋₆alkylcarbonyl (—C(O)C₁₋₆alkyl), such as methylcarbonyl, ethylcarbonyl, n-propylcarbonyl, isopropylcarbonyl, n-butylcarbonyl, isobutylcarbonyl, tert-butylcarbonyl, n-pentylcarbonyl, isopentylcarbonyl, and hexylcarbonyl, with methylcarbonyl being preferable.

The term “alkylamino” is used herein to refer to a moiety derived from an amino (NH₂) moiety in which one or both hydrogen atom(s) of the amino is/are substituted with one or two alkyl moieties. Examples of alkylamino groups include dimethylamino, diethylamino and the like.

As used herein, the term “alkylcarbonylaminoalkyl” refers to aminoalkyl having alkylcarbonyl mentioned above. Examples of alkylcarbonylaminoalkyl include C₁₋₆alkylcarbonylaminoalkyl, such as methylcarbonylaminomethyl and ethylcarbonylaminomethyl.

As used herein, the term “alkylaminocarbonyl” refers to carbonyl having at least one alkylamino group. Examples of alkylaminocarbonyl include C₁₋₆alkylamino-C₁₋₆alkyl, such as methylaminocarbonyl, and ethylaminocarbonyl.

As used herein, the term “alkylaminoalkyl” refers to alkyl mentioned above having at least one alkylamino group mentioned above. Examples of alkylaminoalkyl include C₁₋₆alkylamino-C₁₋₆alkyl, such as methylaminomethyl, methylaminoethyl, ethylaminomethyl, and ethylaminopropyl.

The term “alkoxyalkylene” is used herein to refer to a moiety derived from an alkylene moiety in which a hydrogen atom at any position of the alkylene is substituted with an alkoxy moiety. Examples of alkoxyalkylene groups include methoxyethylene, methoxymethylene and the like.

The term “haloalkylene” is used herein to refer to a moiety derived from an alkylene moiety in which one or more hydrogen atom(s) at any position(s) of the alkylene is/are substituted with one or more halo moieties. Examples of haloalkylene groups include fluoroethylene, difluoromethoxymethylene, dichloroethylene and the like.

The term “hydroxyalkylene” is used herein to refer to a moiety derived from an alkylene moiety in which a hydrogen atom at any position of the alkylene is substituted a hydroxy moiety. Examples of hydroxyalkylene groups include hydroxyethylene, hydroxymethylene and the like.

The term “cycloalkoxy” is used herein to refer to a moiety derived from a linear alkoxy moiety in which a bond forms between the oxygen atom of the OH moiety and the carbon atom at the end of the alkyl chain (by abstraction of the hydrogen atom of the OH moiety and a hydrogen atom at the end of the alkyl chain). Examples of cycloalkoxy groups include oxacyclohexanyl, oxacyclopentanyl and the like.

The term “carbocyclylamino” is used herein to refer to a moiety derived from an linear monohydrocarbylamino moiety in which a bond forms between the nitrogen atom of the NH moiety and the carbon atom at the end of the hydrocarbyl chain (by abstraction of the hydrogen atom of the NH moiety and a hydrogen atom at the end of the hydrocarbyl chain). Examples of carbocyclylamino groups include piperidinyl, pyrrolidinyl, pyridinyl, pyrrolyl and the like.

As used herein, the term “substituted” means that the moiety comprises one or more substituents. As used herein, the “optionally substituted” means that the moiety may comprise one or more substituents.

As used herein, a “substituent” may include, but is not limited to, hydroxy, thiol, carboxyl, cyano (CN), nitro (NO₂), halo, haloalkyl (e.g. a C₁ to C₆ haloalkyl or a C₁ to C₄ haloalkyl), an alkyl group (e.g. C₁ to C₁₀ or C₁ to C₆), an alkenyl group (e.g. C₂ to C₆), an alkynyl group (e.g. C₂ to C₆), aryl (e.g. phenyl and substituted phenyl for example benzyl or benzoyl), morpholino, N—C₁₋₆alkylenylmorpholine, alkoxy group (e.g. C₁ to C₆ alkoxy or C₁ to C₄ alkoxy), haloalkoxy (e.g. C₁ to C₄ haloalkoxy), aryloxy (e.g. phenoxy and substituted phenoxy), hydroxyalkynyl (e.g. C₂ to C₆). thioether (e.g. C₁ to C₆ alkyl or aryl thioether), alkylthio (e.g. C₁ to C₆alkylthio), cyanoalkyl (e.g. C₁ to C₆), oxo, keto (e.g. C₁ to C₆ keto), ester (e.g. C₁ to C₆ alkyl or aryl ester, which may be present as an oxyester or carbonylester on the substituted moiety), thioester (e.g. C₁ to C₆ alkyl or aryl thioester), alkylene ester (such that attachment is on the alkylene group, rather than at the ester function which is optionally substituted with a C₁ to C₆ alkyl or aryl group), amine (including monoalkylamino, dialkylamino, a five- or six-membered cyclic alkylene amine optionally substituted with one or more halo, further including a C₁ to C₆ alkyl amine or a C₁ to C₆ dialkyl amine which alkyl groups may be substituted with one or two hydroxyl groups, and also including alkylphenylamino or alkylphenyl(alkyl)amino groups), amido (including —C(O)NH₂, —C(O)NH(alkyl) such as —C(O)NH(C₁₋₄alkyl), —C(O)N (alkyl)₂ such as —C(O)N(C₁₋₄alkyl)₂, —NHC(O)alkyl such as —NHC(O)C₁₋₄alkyl, —NHC(O)(phenyl), —N(alkyl)C(O)(alkyl) such as —N(C₁₋₄alkyl)C(O)(C₁₋₄alkyl), —N(alkyl)C(O)(phenyl) such as —N(C₁₋₄alkyl)C(O)(phenyl), N—C₁₋₆alkylenylamino, amido (e.g. which may be substituted with one or two C₁ to C₆ alkyl groups (including a carboxamide which is optionally substituted with one or two C₁ to C₆ alkyl groups), aminoalkyl (e.g. C₁ to C₄ aminoalkyl), alkanol (e.g. C₁ to C₆ alkyl, C₁ to C₄ alkyl or aryl alkanol), or carboxylic acid (e.g. C₁ to C₆ alkyl or aryl carboxylic acid), sulfoxide, sulfone, sulfinimide, sulfonamide, and urethane (such as —O—C(O)—NR₂ or —N(R)—C(O)—O—R, wherein each R in this context is independently selected from C₁ to C₆ alkyl or aryl), a heteroaryl, arylalkyl (such as an arylC₁₋₄alkyl), heteroarylalkyl (such as a heteroarylC₁₋₄alkyl), —OC₁₋₄alkylphenyl, —C(O)alkyl such as —C(O)(C₁₋₄alkyl), —C(O)alkylphenyl such as C(O)(C₁₋₄alkylphenyl), —C(O)haloalkyl such as —C(O)(C₁₋₄haloalkyl), —SO₂(alkyl) such as —SO₂(C₁₋₄alkyl), —SO₂(phenyl), —SO₂haloalkyl such as OSO₂(C₁₋₄haloalkyl), —SO₂NH₂, —SO₂NH(alkyl) such as —SO₂NH(C₁₋₄alkyl), —SO₂NH(phenyl), —NHSO₂(alkyl) such as —NHSO₂(C₁₋₄alkyl), —NHSO₂(phenyl), —NHSO₂(haloalkyl) such as —NHSO₂(C₁₋₄haloalkyl), —S—C₁₋₃haloalkyl, —CH₂C(O)N(R^{C})₂, —C₃₋₄alkynyl(NR^{C})₂, deuteroC₂₋₄alkynyl, (C₁₋₃alkoxy)haloC₁₋₃alkyl-, C₃₋₆cycloalkyl (wherein said C₃₋₆cycloalkyl is optionally substituted with halo or C₁₋₃alkyl), HC(O)—, —CO₂R^{C}, or —CO₂N(R^{C})₂, wherein R^{C} is hydrogen or C₁₋₃alkyl.

In some examples, and unless the context indicates otherwise, a “substituent” may include, but is not limited to, halo, C₁ to C₆ alkyl, C₁ to C₆ haloalkyl, C₁ to C₆ alkoxy, hydroxyl, oxo, amino (such as NR¹′R²′), amido (such as —(C═O)NR¹′R²′ or NR¹′ (C═O)C₁-C₆ alkyl), keto (such as —(C═O)C₁-C₆ alkyl), and ester (such as —O(C═O)C₁-C₆ alkyl or —(C═O)OC₁-C₆ alkyl); and wherein R¹′ and R²′ are each independently selected from H and C₁ to C₆ alkyl.

In further examples, and unless the context indicates otherwise, a “substituent” may include, but is not limited to, halo, C₁ to C₆ alkyl, C₁ to C₆ haloalkyl and C₁ to C₆ alkoxy.

As used herein, a “halo” group may be F, Cl, Br, or I. In some examples, halo may be F.

As used herein, “haloalkyl” may be an alkyl group in which one or more hydrogen atoms thereon have been replaced with a halogen atom, e.g. a C₁-C₆ haloalkyl may be a C₁ to C₆ alkyl in which one or more hydrogen atoms thereon have been replaced with a halogen atom. By way of a representative example, a C₁-C₆ haloalkyl may be a fluoroalkyl, such as trifluoromethyl (—CF₃) or 1,1-difluoroethyl (—CH₂CHF₂).

As used herein, a cyclohaloalkyl refers to a cycloalkyl as defined above, in which one or more hydrogen atoms thereon have been replaced with a halogen atom. By way of example, a “C₃ to C₇ cyclohaloalkyl” refers to a C₃ to C₇ cycloalkyl in which one or more hydrogen atoms thereon have been replaced with a halogen atom.

As used herein, a “C₁₋₄ haloalkoxy” refers to a C₁₋₄ alkoxy as defined above, in which one or more hydrogen atoms thereon have been replaced with a halogen atom.

As used herein, the terms “aryl”, “substituted aryl”, “heteroaryl”, “substituted heteroaryl”, “cycloalkyl”, “C₁ to C₆ alkyl”, “heterocycloalkyl”, and “substituted heterocycloalkyl” may refer to either a monovalent radical species or a divalent radical species. For example, within the context of the various formulae for Z described herein, R¹ is typically a monovalent group that is attached to the heterocyclic core of Z and so the terms aryl, substituted aryl, heteroaryl, substituted heteroaryl, cycloalkyl and C₁ to C₆ alkyl should be understood to represent a monovalent radical moiety. By way of further example, R² for Z is typically a divalent group that is covalently attached to both the heterocyclic core of Z and also the linker. As such, in these examples, the terms aryl, substituted aryl, heteroaryl, substituted heteroaryl, heterocycloalkyl, substituted heterocycloalkyl should be understood to represent divalent radical moiety.

It should be understood that throughout this specification, the terms “comprise”, “comprising” and/or “comprises” is/are used to denote that aspects, embodiments and examples of this disclosure “comprise” a particular feature or features. It should be understood that this/these terms may also encompass aspects, embodiments and/or examples which “consist essentially of” or “consist of” the relevant feature or features.

### EXAMPLES

The present invention will now be described in detail with reference to the following non-limiting examples.

### LIST OF ABBREVIATIONS

μL=Microliter
μM=Micromolar
NMR=Nuclear Magnetic Resonance
ACN=acetonitrile
AcOH or HOAc=acetic acid
Boc=tert-butoxycarbonyl
bs=broad singlet
° C.=degrees Celsius
d=doublet
δ=chemical shift
DCM=Dichloromethane
DIPEA=N, N-Diisopropylethylamine, or Hünig's base
DMF=N, N-dimethylformamide
DMSO=Dimethylsulfoxide
dppf=1, 1′-Ferrocenediyl-bis(diphenylphosphine)
EtOAc=Ethyl acetate
g or G=gram
h or H=Hour(s)
HATU=1-[Bis(dimethylamino)methylene]-1H-1,2,3-triazolo[4,5-b]pyridinium 3-oxide hexafluorophosphate
HPLC=high performance liquid chromatography
Hz=Hertz
J=coupling constant (given in Hz unless otherwise indicated)
LCMS=liquid chromatography mass spectrometry
m=multiplet
M=Molar
M+H⁺=parent mass spectrum peak plus H⁺
mg=Milligram
min=minutes
mL=Milliliter
mM=Millimolar
mmol=Millimole
MS=mass spectrum
MTBE=methyl tert-butyl ether
nM=nanomolar
q=quartet
RT or r.t.=room temperature
t=triplet
TFA=trifluoroacetic acid
THF=tetrahydrofuran
TLC=thin layer chromatography

### Chemistry—Materials and Methods

All chemicals, unless otherwise stated were commercially available and used without further purification. Solvents were anhydrous and reactions preformed under positive pressure of nitrogen or argon.

Flash column chromatography (FCC) was performed using a Teledyne Isco Combiflash Rf or Rf200i. Prepacked columns RediSep Rf Normal Phase Disposable Columns were used.

NMR data was acquired in Bruker Avance Neo nano bay 400 MHz NMR Spectrometer. Chemical Shifts are reported in ppm relative to dimethyl Sulfoxide (δ 2.50), methanol (δ 3.31), chloroform (δ 7.26) or other solvent as indicated in NMR spectral data. A small amount (1-5 mg) of sample is dissolved in an appropriate deuterated solvent (0.6 ml).

Preparative HPLC was performed on a Gilson Preparative HPLC System with a Waters X-Bridge C18 column (100 mm×19 mm; 5 μm particle size) and a gradient of 5% to 95% acetonitrile in water over 10 min, flow 25 mL/min, with 0.1% formic acid in the aqueous phase.

Liquid Chromatography Mass Spectra (LC-MS) were recorded using positive ion electron spray ionisation (ESI⁺) on an Agilent InfinityLab Single Quadrupole LC/MSD with a Waters XBridge® C18 3.5 μm column (2.1 mm×50 mm) using H₂O+MeCN (5-95%)+0.1% HCO₂H or H₂O+MeCN (20-95%)+0.1% HCO₂H as eluent, using a linear gradient over 3 minutes. Alternatively, a Shimadzu LC; Prominence-I series instrument was used, with the following set up (unless otherwise stated):

| Column: X-Bridge C8 (150 × 4.6 mm, 5.0 μm) | |
|---|---|
| Column Temperature: Ambient | |
| Detection: UV @ 210-400 nm(Max Plot) | |
| Sample Diluent: Acetonitrile and Water | |
| Mobile Phase A: 10 mM Ammonium Acetate in water | |
| Mobile Phase B: Acetonitrile | |
| Flow rate: 1.5 mL/Min | |
| Runtime: 12.0 Min | |
| Elution: Gradient elution | |
| Time in Min | % of Mobile Phase B |
| 0.01 | 10 |
| 8.0 | 100 |
| 10.0 | 100 |
| 10.01 | 10 |
| 12.0 | 10 |

HPLC Method B

| Column: X-select CSH C18 (150 × 4.6 mm, 5.0 μm) | |
|---|---|
| Column Temperature: Ambient | |
| Detection: UV @ 210-400 nm(Max Plot) | |
| Sample Diluent: Acetonitrile and Water | |
| Mobile Phase A: 0.1% Formic acid in water | |
| Mobile Phase B: Acetonitrile | |
| Runtime: 10.0 Min | |
| Flowrate: 2.0 mL/Min | |
| Elution: Gradient elution | |
| Time in Min | % of Mobile Phase B |
| 0 | 5 |
| 8 | 100 |
| 8.01 | 5 |
| 10 | 5 |

### PART A—Synthetic Methods

### BET Inhibitor (BRD4) Degraders

Overviews of various exemplary synthetic methods that may be used to provide the compounds of the present disclosure are shown below.

### Overview of Synthetic Pathway—Scheme 1

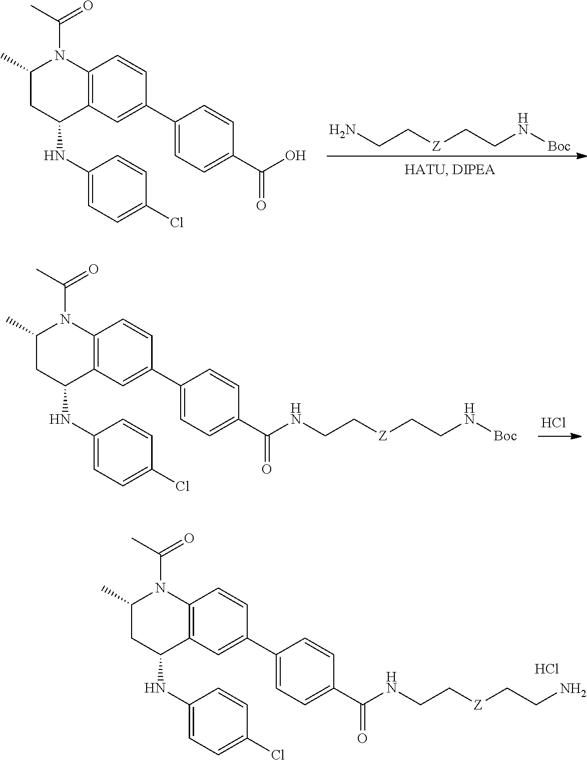

### Overview of Synthetic Pathway—Scheme 2

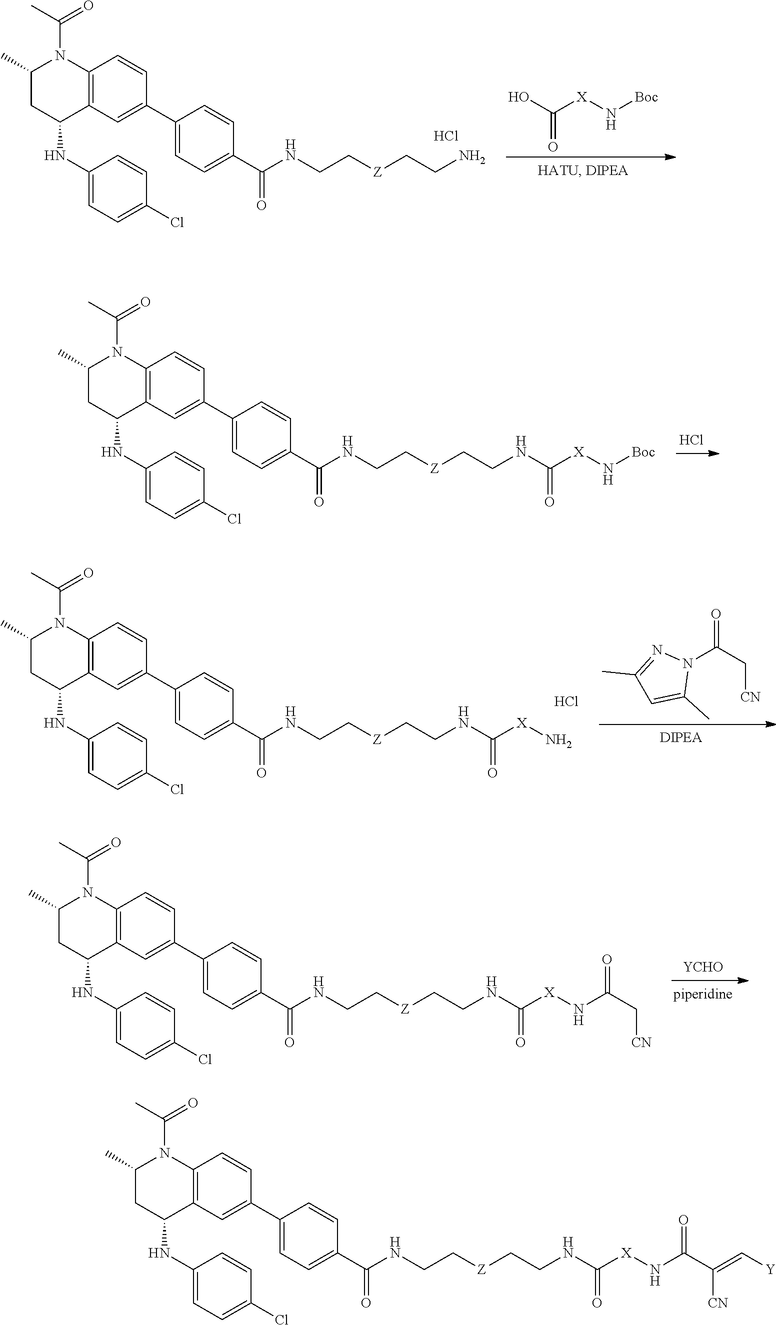

### Overview of Synthetic Pathway—Scheme 3

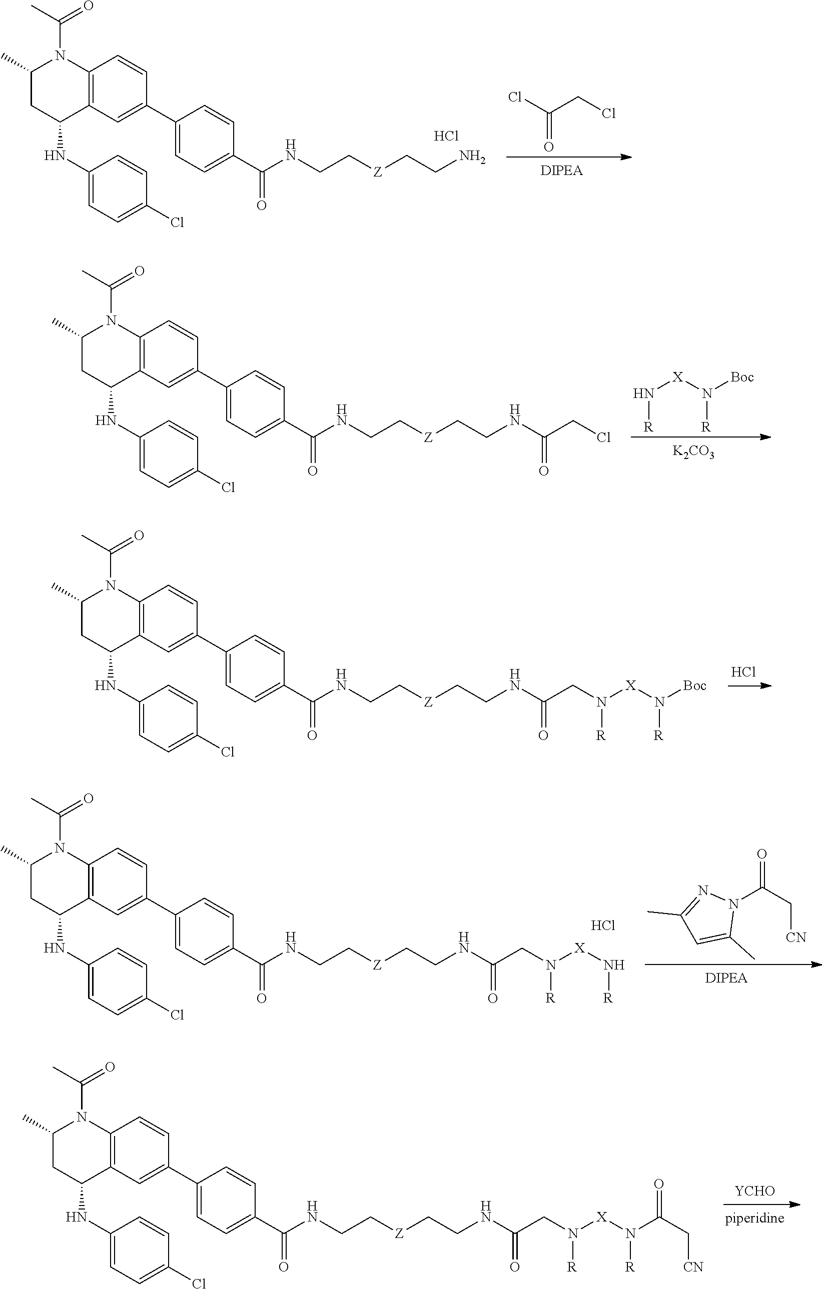

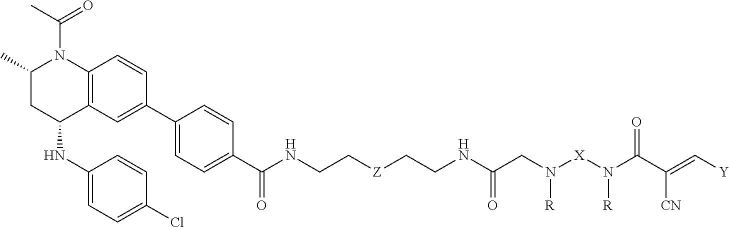

### Overview of Synthetic Pathway—Scheme 4

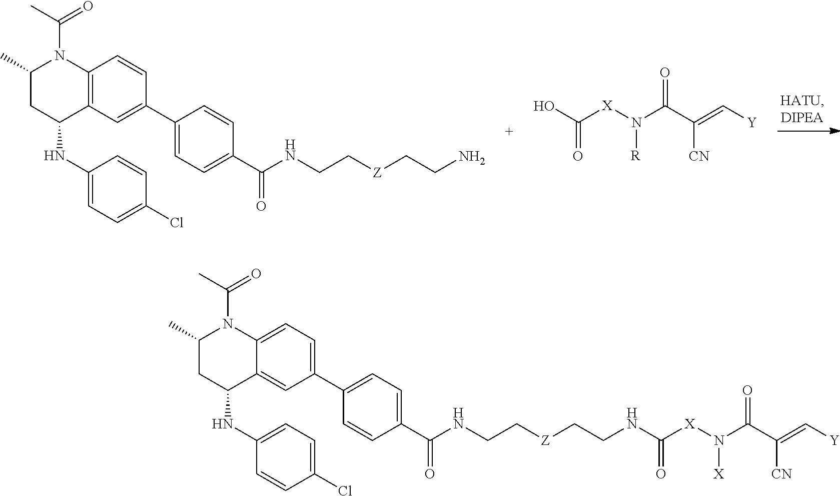

### Example 1a: tert-butyl (5-(4-((2S,4R)-1-acetyl-4-((4-chlorophenyl)amino)-2-methyl-1,2,3,4-tetrahydroquinolin-6-yl)benzamido)pentyl)carbamate: (see scheme 1)

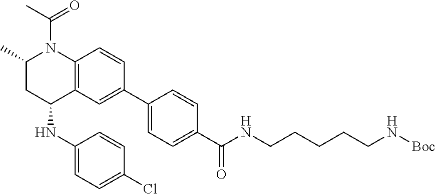 [SMILES: CC(=O)N1c2ccc(-c3ccc(C(=O)NCCCCCNC(=O)OC(C)(C)C)cc3)cc2[C@H](Nc2ccc(Cl)cc2)C[C@@H]1C]

To a stirred solution of commercially available GSK1324726A (1.1 equiv., 5.9 g, 13.6 mmol) in DMF (25 ml) were added DIPEA (3 equiv., 4.79 g, 6.4577 mL, 37.075 mmol) and HATU (1.1 equiv., 5.17 g, 13.6 mmol) at 0° C. and stirred for 10 min. To this reaction mixture was added tert-butyl N-(5-aminopentyl) carbamate (1.0 equiv., 2.5 g, 12.4 mmol) dissolved in DMF (5 mL) at 0° C. and stirred for additional 24 h at RT. The progress of the reaction was monitored by LCMS. Reaction was quenched with ice cold water; and precipitation was observed. The precipitate was filtered off, the solid obtained was washed by water and dried under reduced pressure. The crude compound was purified by silica gel column chromatography using EtOAc/Hexane as eluents to afford the title compound (6.8 g, 8.97 mmol, 72.6% yield) as an off-white solid. m/z=519.2 [M−Boc+H]⁺

### Additional Examples

An additional example made in accordance with a method similar to that described above (and illustrated in scheme 1 is shown below).

| Example | | |
|---|---|---|
| number | Structure/Preparation | m/z (M + H)⁺ |
| 1b | 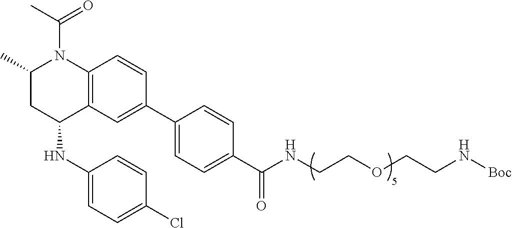 | 820.2 [M + Na]⁺ |

### Example 2a: 4-((2S,4R)-1-acetyl-4-((4-chlorophenyl)amino)-2-methyl-1,2,3,4-tetrahydroquinolin-6-yl)-N-(5-aminopentyl)benzamide hydrochloride: (see scheme 1)

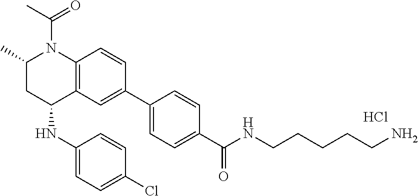 [SMILES: CC(=O)N1c2ccc(-c3ccc(C(=O)NCCCCCN)cc3)cc2[C@H](Nc2ccc(Cl)cc2)C[C@@H]1C ; Cl]

To a stirred solution of tert-butyl (5-(4-((2S,4R)-1-acetyl-4-((4-chlorophenyl)amino)-2-methyl-1,2,3,4-tetrahydroquinolin-6-yl)benzamido) pentyl) carbamate (1a) (4.5 g, 7.27 mmol) in DCM (100 ml) was added HCl (4M in dioxane) (18.2 ml, 10.0 equiv. 72.7 mmol) slowly at 0° C. The reaction was allowed to stir at RT for 2 h. The progress of the reaction was monitored by TLC. After completion, the reaction mixture was concentrated under reduced pressure to get a crude solid, which was washed with MTBE (100 ml) and dried under vacuum, to afford 4-((2S,4R)-1-acetyl-4-((4-chlorophenyl)amino)-2-methyl-1,2,3,4-tetrahydroquinolin-6-yl)-N-(5-aminopentyl)benzamide hydrochloride (4.2 g, 6.77 mmol, 93% yield). The crude was used for the next step without further purification. m/z=519.0 [M+H]⁺.

### Additional Examples

An additional example made in accordance with a method similar to that described above (carried out on compound 1b) (and illustrated in scheme 1 is shown below).

| Example | | |
|---|---|---|
| number | Structure/Preparation | m/z (M + H)⁺ |
| 2b | 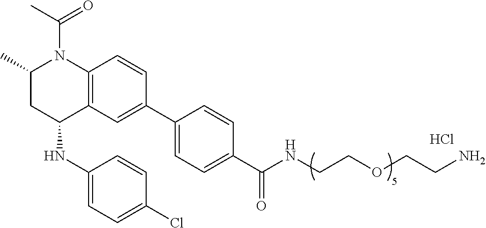 | 697.8 [M + H]⁺ |

### Example 3:4-((2S,4R)-1-acetyl-4-((4-chlorophenyl)amino)-2-methyl-1,2,3,4-tetrahydroquinolin-6-yl)-N-(1-chloro-2-oxo-6,9, 12, 15, 18-pentaoxa-3-azaicosan-20-yl)benzamide (see scheme 3)

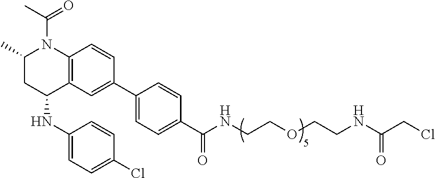

To a stirred solution of 4-((2S,4R)-1-acetyl-4-((4-chlorophenyl)amino)-2-methyl-1,2,3,4-tetrahydroquinolin-6-yl)-N-(5-aminopentyl)benzamide hydrochloride (2b) (120 mg, 0.164 mmol) in dichloromethane (DCM) (0.5 ml) at 0° C. were sequentially added Et₃N (60.8 μL, 5.0 equiv. 0.436 mmol) and chloroacetyl chloride (20.8 μL, 3.0 equiv. 0.262 mmol. The reaction was allowed to stir at room temperature for 16 h. The progress of the reaction was monitored by LC-MS. After completion, the reaction was quenched by addition of NH₄Cl (saturated aqueous solution) the mixture was extracted with DCM (10 mL). The organic phase was dried over MgSO₄ and concentrated under reduced pressure. Purification by column chromatography (SiO₂, DCM/MeOH) yielded 4-((2S,4R)-1-acetyl-4-((4-chlorophenyl)amino)-2-methyl-1,2,3,4-tetrahydroquinolin-6-yl)-N-(1-chloro-2-oxo-6,9, 12, 15, 18-pentaoxa-3-azaicosan-20-yl)benzamide (3) (77.0 mg, 0.0995 mmol, 61.0% yield). m/z=773.8 [M+H]⁺

### Example 4: tert-butyl 2-(1-(1-(4-((2S,4R)-1-acetyl-4-((4-chlorophenyl)amino)-2-methyl-1,2,3,4-tetrahydroquinolin-6-yl)phenyl)-1,21-dioxo-5,8, 11, 14, 17-pentaoxa-2,20-diazadocosan-22-yl) piperidin-4-yl)pyrrolidine-1-carboxylate (see scheme 3)

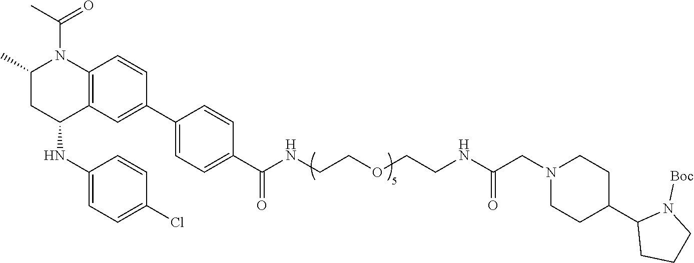

To a stirred solution of 4-((2S,4R)-1-acetyl-4-((4-chlorophenyl)amino)-2-methyl-1,2,3,4-tetrahydroquinolin-6-yl)-N-(1-chloro-2-oxo-6,9, 12, 15, 18-pentaoxa-3-azaicosan-20-yl)benzamide (3) (77.0 mg, 0.0995 mmol) in DMF (0.5 ml)) at 0° C. were sequentially added tert-butyl 2-(piperidin-4-yl)pyrrolidine-1-carboxylate (3.00 equiv., 76 mg, 0.299 mmol) and Potassium Carbonate (3.00 equiv., 41 mg, 0.299 mmol) and heated to 80° C. for 12 h. After cooling to room temperature the mixture was diluted with EtOAc. The organic phase was washed with LiCl (5% aqueous solution), dried over MgSO₄ and concentrated under reduced pressure. Purification by column chromatography (SiO₂, DCM/MeOH) yielded tert-butyl 2-(1-(1-(4-((2S,4R)-1-acetyl-4-((4-chlorophenyl)amino)-2-methyl-1,2,3,4-tetrahydroquinolin-6-yl)phenyl)-1,21-dioxo-5,8, 11, 14, 17-pentaoxa-2,20-diazadocosan-22-yl) piperidin-4-yl)pyrrolidine-1-carboxylate (80.0 mg, 0.0807 mmol, 81.1% yield). m/z=992.2 [M+H]⁺

### Example 5a: tert-butyl 3-((5-(4-((2S,4R)-1-acetyl-4-((4-chlorophenyl)amino)-2-methyl-1,2,3,4-tetrahydroquinolin-6-yl)benzamido)pentyl)carbamoyl)-5-(m-tolyl)piperidine-1-carboxylate (see, for example, scheme 2)

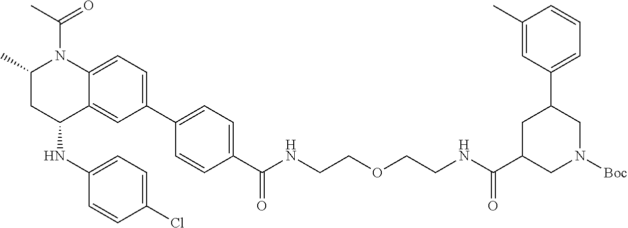 [SMILES: CC(=O)N1c2ccc(-c3ccc(C(=O)NCCOCCNC(=O)C4CC(c5cccc(C)c5)CN(C(=O)OC(C)(C)C)C4)cc3)cc2[C@H](Nc2ccc(Cl)cc2)C[C@@H]1C]

To a stirred solution of 1-(tert-butoxycarbonyl)-5-(m-tolyl)piperidine-3-carboxylic acid (0.095 g, 0.297 mmol) in 1 mL DMF was added DIPEA (0.189 ml, 1.080 mmol) followed by HATU (0.154 g, 0.405 mmol) in 1 mL DMF. Resulting solution was stirred for 10 min before the addition of 4-((2S,4R)-1-acetyl-4-((4-chlorophenyl)amino)-2-methyl-1,2,3,4-tetrahydroquinolin-6-yl)-N-(5-aminopentyl)benzamide hydrochloride (2a) (0.15 g, 0.270 mmol) in 1 ml of DMF and stirred for overnight. Reaction progress was monitored by LCMS. The reaction mixture was diluted by cold water (5 mL) and extracted with EtOAc (3×10 mL), the organic layer was washed with water and brine, dried over Na₂SO₄ and concentrated to afford crude tert-butyl 3-((5-(4-((2S,4R)-1-acetyl-4-((4-chlorophenyl)amino)-2-methyl-1,2,3,4-tetrahydroquinolin-6-yl)benzamido)pentyl)carbamoyl)-5-(m-tolyl)piperidine-1-carboxylate (0.2 g, 0.214 mmol, 79% yield). m/z=720 [M−Boc+H]⁺.

### Additional Examples

Additional examples made in accordance with a method similar to that described above and illustrated in scheme 2 are shown below (carried out on compounds 2a and 2b with various carboxylic acids)

| Example | | m/z |
|---|---|---|
| number | Structure/Preparation | (M + H)⁺ |
| 5b | 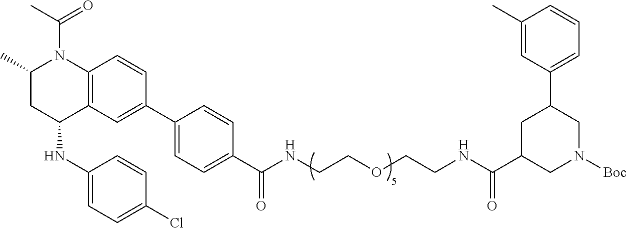 | [M]⁺ 1060.2 and [M + 2]⁺ 1062.1 |
| 5c | 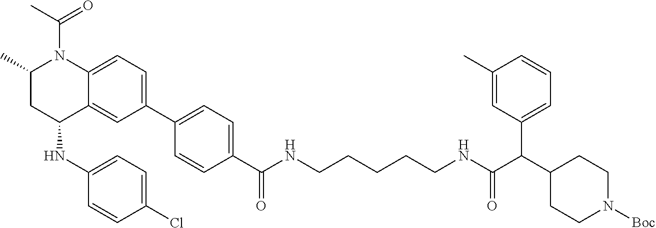 [SMILES: CC(=O)N1c2ccc(-c3ccc(C(=O)NCCCCCNC(=O)C(c4cccc(C)c4)C4CCN(C(=O)OC(C)(C)C)CC4)cc3)cc2[C@H](Nc2ccc(Cl)cc2)C[C@@H]1C] | [M]⁺ 834.0 |
| 5d | 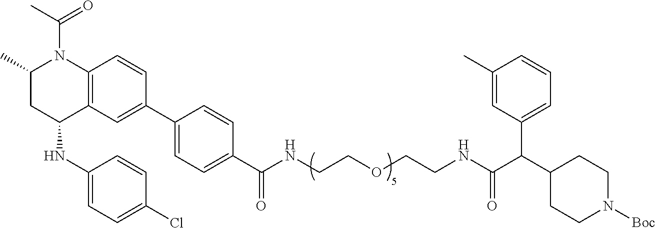 | [M]⁺ 1074.4 and [M + 2]⁺ 1076.4 |
| 5e | 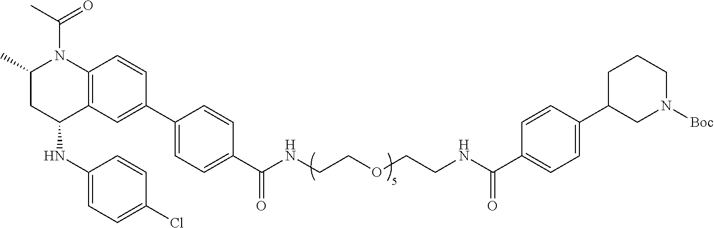 | [M + H]⁺ 985.3 and [M + Na]⁺ 1007.3 |
| 5f | 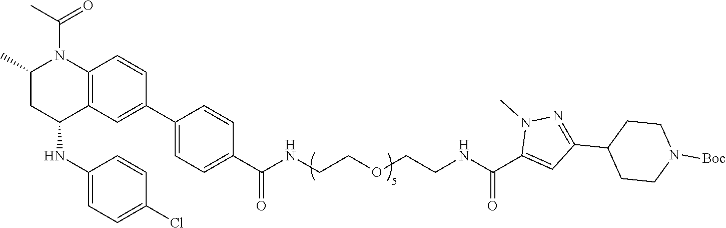 | [M + H]⁺ 1111.3 and [M + Na]⁺ 1133.2 |
| 5g | 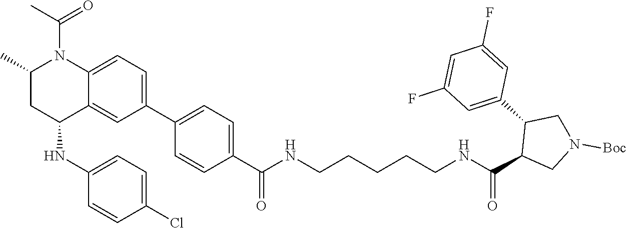 [SMILES: CC(=O)N1c2ccc(-c3ccc(C(=O)NCCCCCNC(=O)[C@@H]4CN(C(=O)OC(C)(C)C)C[C@H]4c4cc(F)cc(F)c4)cc3)cc2[C@H](Nc2ccc(Cl)cc2)C[C@@H]1C] | [M − 100]⁺ 728.2 |

### Example 6a: N-(5-(4-((2S,4R)-1-acetyl-4-((4-chlorophenyl)amino)-2-methyl-1,2,3,4-tetrahydroquinolin-6-yl)benzamido)pentyl)-5-(m-tolyl)piperidine-3-carboxamide hydrochloride (see for example, scheme 3)

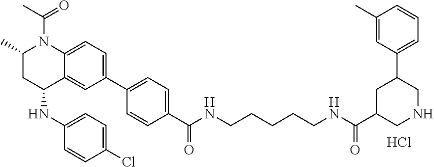 [SMILES: CC(=O)N1c2ccc(-c3ccc(C(=O)NCCCCCNC(=O)C4CNCC(c5cccc(C)c5)C4)cc3)cc2[C@H](Nc2ccc(Cl)cc2)C[C@@H]1C ; Cl]

To a solution of tert-butyl 3-((5-(4-((2S,4R)-1-acetyl-4-((4-chlorophenyl)amino)-2-methyl-1,2,3,4-tetrahydroquinolin-6-yl)benzamido) pentyl) carbamoyl)-5-(m-tolyl) piperidine-1-carboxylate (1 equiv., 222 mg, 0.271 mmol) in DCM (3 mL, 0.0901 M) was added HCl solution (4M in 1,4-dioxane) (10 equiv., 98.6 mg, 2.71 mmol) slowly at 0° C. and then warmed reaction slowly to room temperature and stirred for 3 h. The reaction progress was monitored by TLC and LCMS. The reaction mixture was concentrated under reduced pressure, washed with MTBE (10 ml) and dried under vacuum to afford the crude N-(5-(4-((2S,4R)-1-acetyl-4-((4-chlorophenyl)amino)-2-methyl-1,2,3,4-tetrahydroquinolin-6-yl)benzamido)pentyl)-5-(m-tolyl)piperidine-3-carboxamide hydrochloride (150 mg, 0.16 mmol, 60.5% yield). The crude was used for the next step without further purification. m/z=720.4 [M+H]⁺

### Additional Examples

| Example | | m/z |
|---|---|---|
| number | Structure/Preparation | (M + H)⁺ |
| 6b | 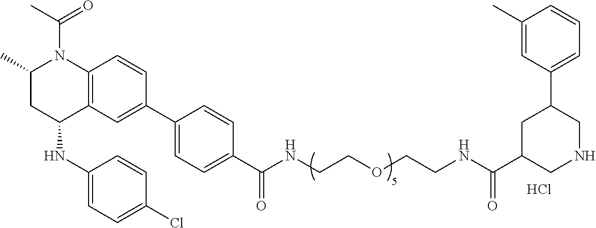 | [M]⁺ 898.2 and [M + 2]⁺ 900.2 |
| 6c | 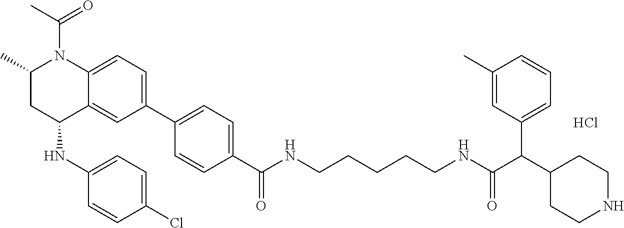 [SMILES: CC(=O)N1c2ccc(-c3ccc(C(=O)NCCCCCNC(=O)C(c4cccc(C)c4)C4CCNCC4)cc3)cc2[C@H](Nc2ccc(Cl)cc2)C[C@@H]1C ; Cl] | [M + H]⁺ 734.3 |
| 6d | 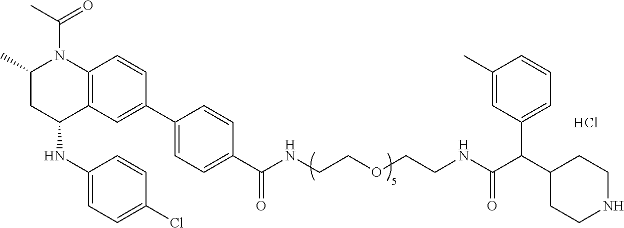 | [M]⁺ 912.4 |
| 6e | 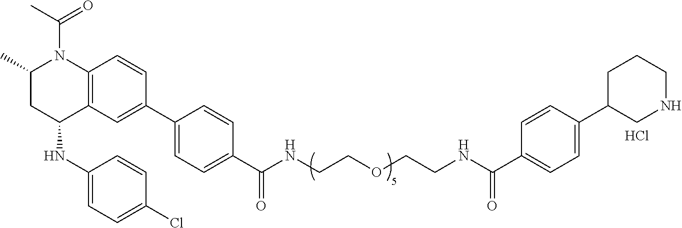 | [M]⁺ 920.2 |
| 6f | 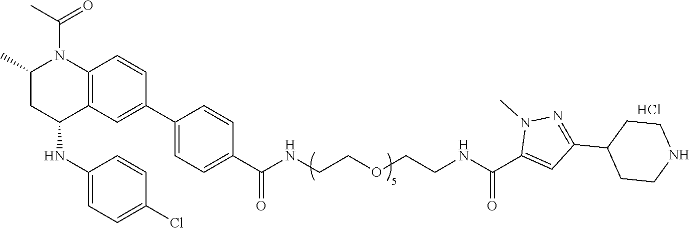 | [M + H]⁺ 889.3 and [M + Na]⁺ 911.3 |
| 6g | 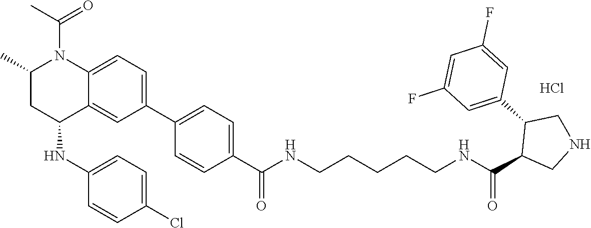 [SMILES: CC(=O)N1c2ccc(-c3ccc(C(=O)NCCCCCNC(=O)[C@@H]4CNC[C@H]4c4cc(F)cc(F)c4)cc3)cc2[C@H](Nc2ccc(Cl)cc2)C[C@@H]1C ; Cl] | [M]⁺ 728.3 and [M + 2]⁺ 730.0 |
| 6h | 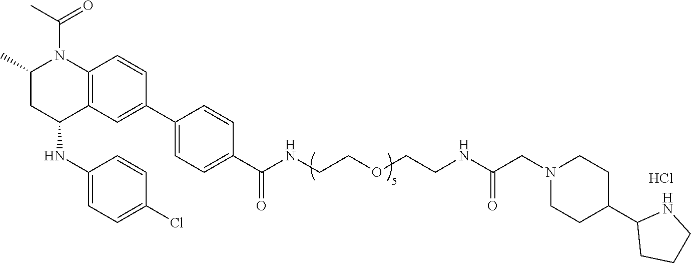 | [M + H]⁺ 892.0 |

### Example 7a: N-(5-(4-((2S,4R)-1-acetyl-4-((4-chlorophenyl)amino)-2-methyl-1,2,3,4-tetrahydroquinolin-6-yl)benzamido)pentyl)-1-(2-cyanoacetyl)-5-(m-tolyl)piperidine-3-carboxamide (see, for example, scheme 2)

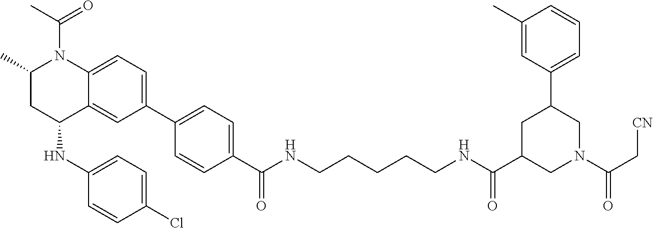 [SMILES: CC(=O)N1c2ccc(-c3ccc(C(=O)NCCCCCNC(=O)C4CC(c5cccc(C)c5)CN(C(=O)CC#N)C4)cc3)cc2[C@H](Nc2ccc(Cl)cc2)C[C@@H]1C]

To a stirred solution N-(5-(4-((2S,4R)-1-acetyl-4-((4-chlorophenyl)amino)-2-methyl-1,2,3,4-tetrahydroquinolin-6-yl)benzamido)pentyl)-5-(m-tolyl)piperidine-3-carboxamide hydrochloride (6a) (1 equiv., 31.5 mg, 0.0416 mmol) in 1,4-dioxane (1 ml) was added DIPEA (3 equiv., 16.2 mg, 0.125 mmol) taken in (0.5 ml) 1,4-dioxane and 1-cyanoacetyl-3,5-dimethyl-1H-pyrazole (2 equiv., 13.6 mg, 0.0833 mmol) taken in (0.5 ml) 1,4-dioxane at room temperature and stirred at 80° C. for 6 h. The crude obtained was dissolved in DCM (20 mL) and washed by H₂O (10 mL×2), the organic layer was dried over Na₂SO₄ and concentrated to give the title compound as a crude product (30.2 mg, 79.9% yield). The crude was used for the next step without further purification. m/z=786.0 [M−H]⁻

### Additional Example

Additional examples made in accordance with a method similar to that described above and illustrated in scheme 2 are shown below (carried out on compounds 6b to 6h).

| Example | | m/z |
|---|---|---|
| number | Structure/Preparation | (M + H)⁺ |
| 7b | 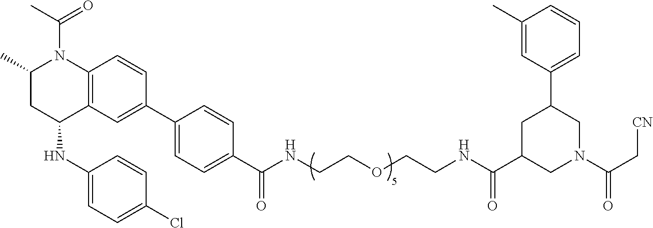 | [M − H]⁻ 800.4 and [M + Na]⁺ 824.3 |
| 7c | 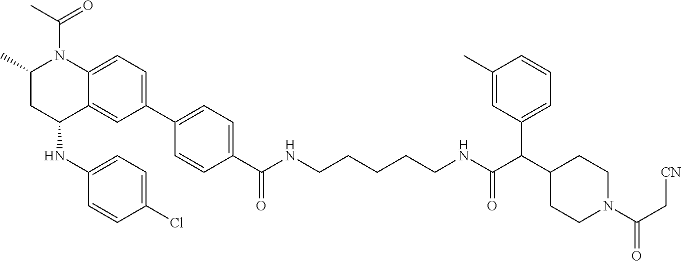 [SMILES: CC(=O)N1c2ccc(-c3ccc(C(=O)NCCCCCNC(=O)C(c4cccc(C)c4)C4CCN(C(=O)CC#N)CC4)cc3)cc2[C@H](Nc2ccc(Cl)cc2)C[C@@H]1C] | [M]⁺ 951.1 and [M + Na]⁺ 974.2 |
| 7d | 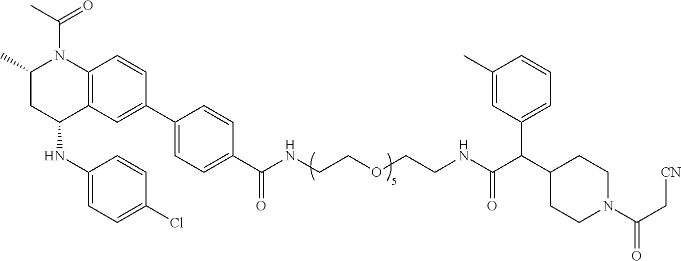 | [M + H]⁺ 980.2 |
| 7e | 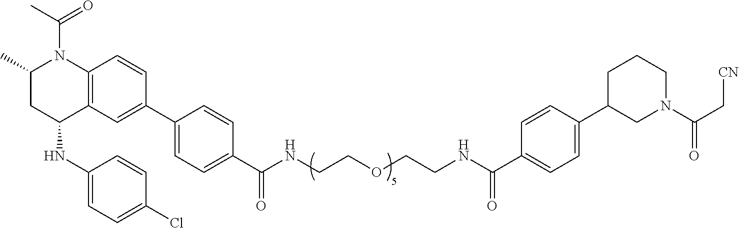 | [M + H]⁺ 966.2 and [M + Na]⁺ 988.2 |
| 7f | 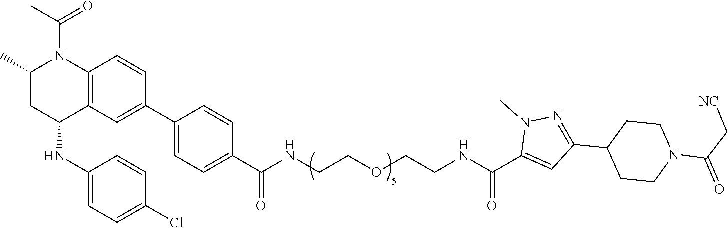 | [M]⁺ 955.5 and [M + 2]⁺ 957.2 |
| 7g | 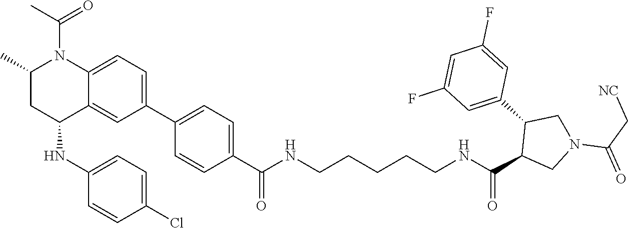 [SMILES: CC(=O)N1c2ccc(-c3ccc(C(=O)NCCCCCNC(=O)[C@@H]4CN(C(=O)CC#N)C[C@H]4c4cc(F)cc(F)c4)cc3)cc2[C@H](Nc2ccc(Cl)cc2)C[C@@H]1C] | [M]⁺795.0 and [M − H]⁺ 794.2 |
| 7h | 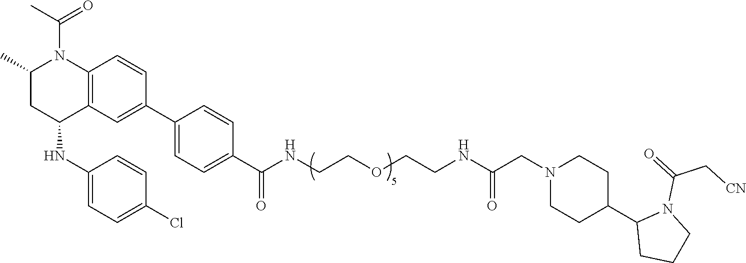 | [M + H]⁺ 959.1 |

### Example 8a: N-(5-(4-((2S,4R)-1-acetyl-4-((4-chlorophenyl)amino)-2-methyl-1,2,3,4-tetrahydroquinolin-6-yl)benzamido)pentyl)-1-(2-cyanoacetyl)-5-(m-tolyl)piperidine-3-carboxamide (see, for example, scheme 2)

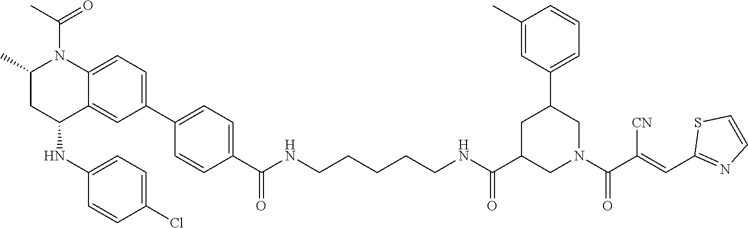 [SMILES: CC(=O)N1c2ccc(-c3ccc(C(=O)NCCCCCNC(=O)C4CC(c5cccc(C)c5)CN(C(=O)/C(C#N)=C/c5nccs5)C4)cc3)cc2[C@H](Nc2ccc(Cl)cc2)C[C@@H]1C]

To a stirred solution of N-(5-(4-((2S,4R)-1-acetyl-4-((4-chlorophenyl)amino)-2-methyl-1,2,3,4-tetrahydroquinolin-6-yl)benzamido) pentyl)-1-(2-cyanoacetyl)-5-(m-tolyl) piperidine-3-carboxamide (7a) (1 equiv., 32.8 mg, 0.0416 mmol) in EtOH (1 mL) at room temperature was added piperidine (1.5 equiv. 0, 5.3 mg, 0.0625 mmol) diluted with 0.5 mL ethanol, followed by 1,3-Thiazole-2-carbaldehyde (3 equiv, 14.1 mg, 0.1249 mmol) taken in 0.5 mL of EtOH. The resulting reaction mixture was stirred overnight at room temperature under nitrogen. The solvent in the reaction mixture was removed under reduced pressure to afford the crude compound. The crude compound was purified by mass directed prep HPLC using ammonium acetate/ACN (Phenomenx c18, 250×21.1 mm, 5u) to afford the title compound (4.6 mg, 0.0052 mmol, 12.4% yield) as off white solid. m/z: 905.1 [M+Na]⁺

### Additional Examples

Additional examples made in accordance with a method similar to that described above and illustrated in scheme 2 are shown below (carried out on compounds 7b to 7h).

| Exam- | | |
|---|---|---|
| ple | | m/z |
| num- | | (M + |
| ber | Structure/Preparation | H)⁺ |
| 8b | 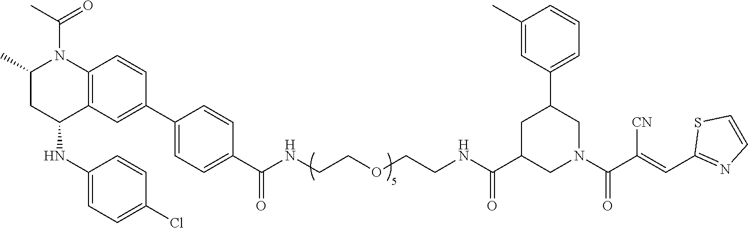 | [M − H]⁻ 895.2 and [M + Na]⁺ 919.2 |
| 8c | 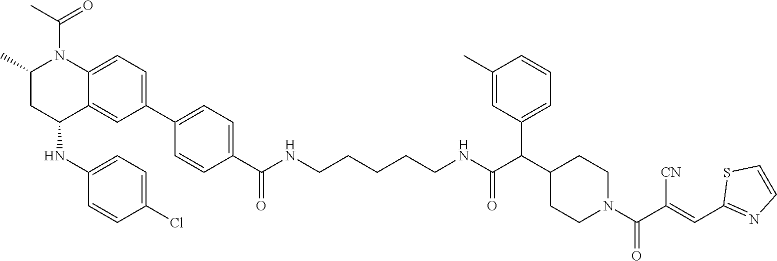 [SMILES: CC(=O)N1c2ccc(-c3ccc(C(=O)NCCCCCNC(=O)C(c4cccc(C)c4)C4CCN(C(=O)/C(C#N)=C/c5nccs5)CC4)cc3)cc2[C@H](Nc2ccc(Cl)cc2)C[C@@H]1C] | [M]⁺ 1046.2 and [M + 23]⁺ 1069.1 |
| 8d | 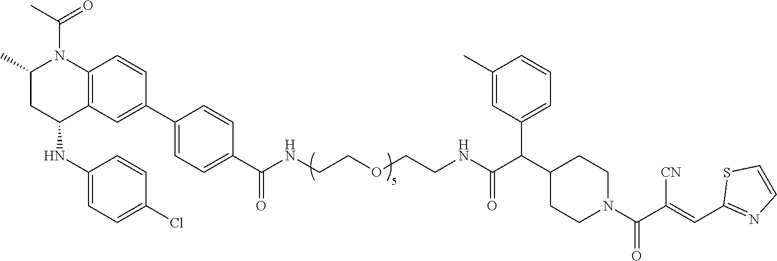 | [M]⁺ 1074.4; [M + 2]⁺ 1076.4; [M + 23]⁺ 1097.3 |
| 8e | 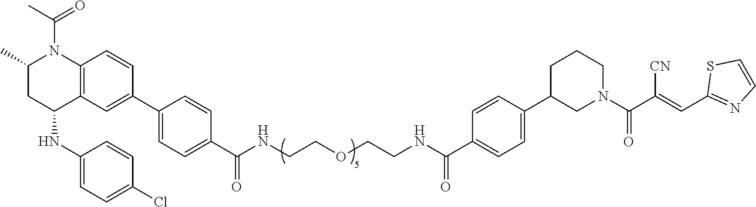 | [M]⁺ 1060.2 and [M + 2]⁺ 1062.1 |
| 8f | 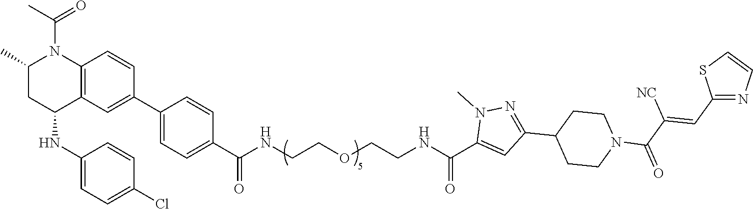 | [M + H]⁺ 1051.2 and [M + Na]⁺ 1073.2 |
| 8g | 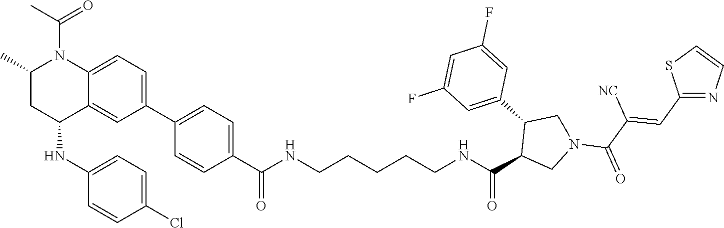 [SMILES: CC(=O)N1c2ccc(-c3ccc(C(=O)NCCCCCNC(=O)[C@@H]4CN(C(=O)/C(C#N)=C/c5nccs5)C[C@H]4c4cc(F)cc(F)c4)cc3)cc2[C@H](Nc2ccc(Cl)cc2)C[C@@H]1C] | [M − H]⁺ 889 and [M + Na]⁺ 913.0 |
| 8h | 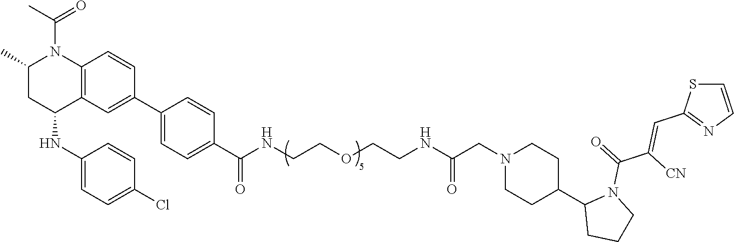 | [M]⁺ 1054.1 |

### Piperidine-Aminoacid Based Warheads

Further examples of bifunctional degraders comprising a piperidine-amino acid derivative-based moiety as Z are illustrated below.

Overviews of various exemplary synthetic methods that may be used to provide these compounds are shown below.

### Overview of Synthetic Pathway—Scheme 5

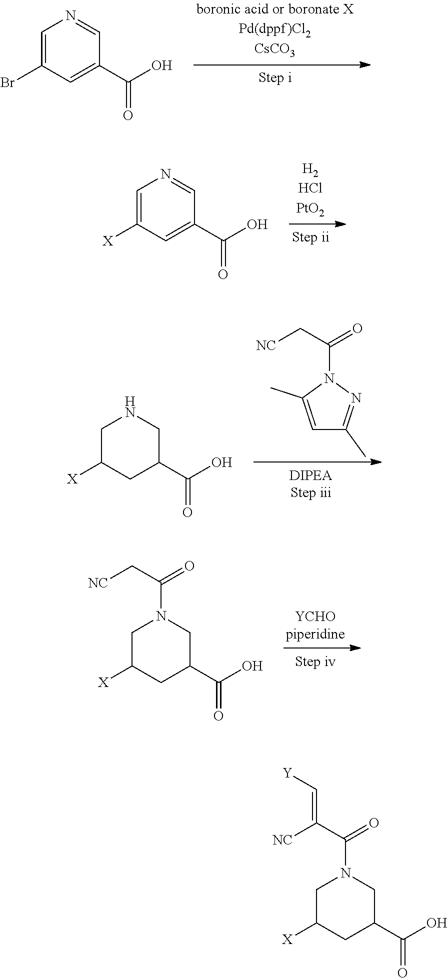

### Overview of Synthetic Pathway—Scheme 6

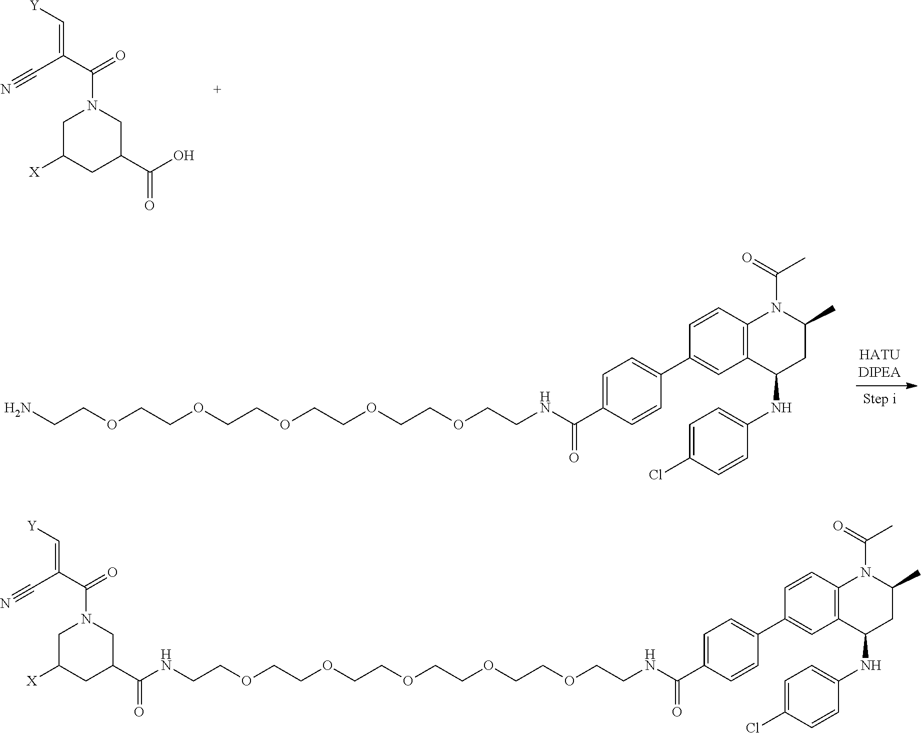

### Synthesis of Piperidine-Amino Acids (See Scheme 5);

5-bromonicotinic acid is treated with the desired boronate or boronic acid X under Suzuki conditions ((PdCl₂ (dppf)·DCM, K₂CO₃, in dioxane/water at 100° C.) in order to obtain the 5 substituted nicotinic acids, unless commercially available. Hydrogenation under (H₂, PtO₂ in HCl or HOAc) affords the substituted nipecotic acids. Acylation using 1 cyanoacetyl-3,5-dimethylpyrazole and DIPEA in dioxane or DMF affords the precursors for the Knoevenagel reaction, which can be carried on using aldehydes Y in ethanol at r.t. (or THF at 40 to 70° C.) using piperidine as catalyst.

### Example 9a: 5-phenylnicotinic acid

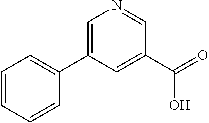 [SMILES: O=C(O)c1cncc(-c2ccccc2)c1]

To a stirred solution of 5-bromonicotinic acid (1.0 equiv., 2.0 g, 9.90 mmol), phenylboronic acid (1.2 equiv., 1.4 g, 11.88 mmol) in Dioxane (20 ml) H₂O (4.0 ml) was added K₂CO₃ (2.0 equiv., 2.7 g, 19.80 mmol). After addition the reaction mixture was degassed with N₂ gas for 20 min and then added catalyst Pd(dppf)Cl₂·CH₂Cl₂ (0.1 equiv., 0.80 g, 0.990 mmol) at RT and then stirred at 100° C. for 16 h. The progress of the reaction was monitored by LCMS. The reaction mixture was filtered through a celite bed and washed with EtOAc and water. The filtrate was concentrated completely and added ice cold water was added. The mixture was extracted with EtOAc. The aqueous layer was acidified with 1.5 N HCl at 0° C. and stirred for 30 min until precipitation was observed. The precipitate was filtered off and washed with water and dried thoroughly to afford 5-phenylnicotinic acid (1.10 g, 5.41 mmol, 54.7% yield) as an off white solid. m/z=200.1 [M+H]⁺; ¹H-NMR (400 MHz, DMSO-d6): δ 13.57 (bs, 1H), 9.09 (dd, J=2.00, 18.60 Hz, 2H), 8.45 (t, J=2.40 Hz, 1H), 7.81-7.78 (m, 2H), 7.56-7.47 (m, 3H).

### Additional Examples

Additional examples made in accordance with a method similar to that described above and illustrated in scheme 5 are shown below.

| Example | | | m/z |
|---|---|---|---|
| number | Employed Boronate | Structure/Preparation | (M + H)⁺ |
| 9b | 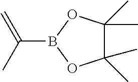 | 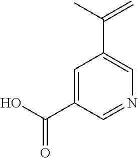 | 164.3 |
| 9c | 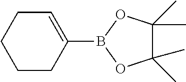 | 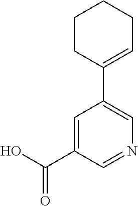 [SMILES: O=C(O)c1cncc(C2=CCCCC2)c1] | 204.2 |
| 9d | 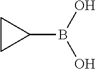 | 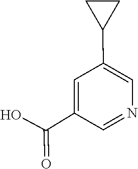 [SMILES: O=C(O)c1cncc(C2CC2)c1] | 164.4 |
| 9e | 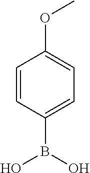 | 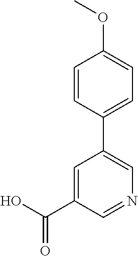 [SMILES: COc1ccc(-c2cncc(C(=O)O)c2)cc1] | 230.1 |
| 9f | 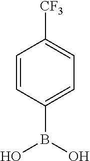 [SMILES: OB(O)c1ccc(C(F)(F)F)cc1] | 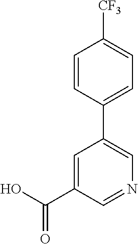 [SMILES: O=C(O)c1cncc(-c2ccc(C(F)(F)F)cc2)c1] | 268.2 |
| 9g | 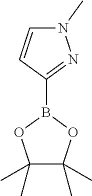 | 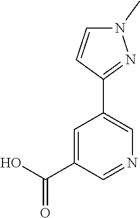 | 204.3 |
| 9i | 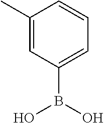 | 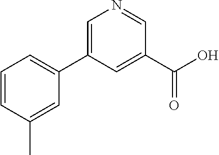 [SMILES: Cc1cccc(-c2cncc(C(=O)O)c2)c1] | 214.1 |

### Example 10a: 5-phenylpiperidine-3-carboxylic acid (see, for example, scheme 5)

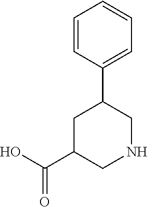 [SMILES: O=C(O)C1CNCC(c2ccccc2)C1]

To a stirred solution of 5-phenylnicotinic acid (1.0 equiv., 0.500 g, 2.510 mmol) in acetic acid (20 ml) was added Platinum (IV) oxide (0.4 equiv., 0.285 g, 1.255 mmol) at room temperature and stirred under hydrogen gas bladder pressure for 48 h. The progress of the reaction was monitored by LCMS. Reaction mass was filtered through celite bed and washed with methanol. The filtrate was concentrated under reduced pressure. The crude residue was purified by reverse phase column chromatography in 0.1% formic acid: acetonitrile to give 5-phenylpiperidine-3-carboxylic acid (0.500 g, 1.949 mmol, 99% yield) as gummy colourless compound. m/z=206.2 [M+H]⁺; ¹H-NMR (400 MHz, DMSO-d6): δ 11.37 (bs, 1H), 7.33-7.21 (m, 5H), 3.17-2.79 (m, 5H), 2.70-2.62 (m, 1H), 1.84-1.66 (m, 2H).

### Additional Examples

Additional examples made in accordance with a method similar to that described above and illustrated in step ii, scheme 5 are shown below.

| Example | | m/z |
|---|---|---|
| number | Structure/Preparation | (M + H)⁺ |
| 10b | 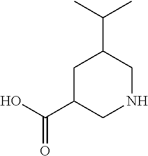 | 172.2 |
| 10c | 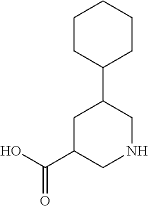 [SMILES: O=C(O)C1CNCC(C2CCCCC2)C1] | 212.3 |
| 10d | 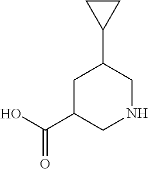 [SMILES: O=C(O)C1CNCC(C2CC2)C1] | 170.1 |
| 10e | 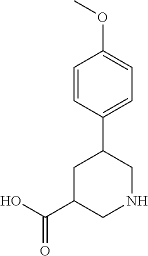 [SMILES: COc1ccc(C2CNCC(C(=O)O)C2)cc1] | 236.2 |
| 10f | 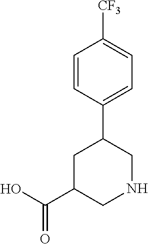 [SMILES: O=C(O)C1CNCC(c2ccc(C(F)(F)F)cc2)C1] | 274.2 |
| 10g | 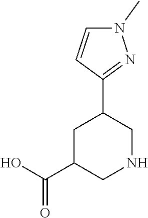 | 210.1 |
| 10i | 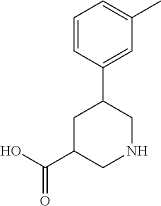 [SMILES: Cc1cccc(C2CNCC(C(=O)O)C2)c1] | 220.1 |
| 10k | 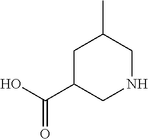 | 144.1 |

N.B. 3-piperidine carboxylic acid is commercially available and, for example, can be obtained from Sigma-Aldrich.

Compounds 10a to 10k and 3-piperidine carboxylic acid are then reacted with DIPEA and 1-cyanoacetyl-3,5-dimethyl-1H-pyrazole (as illustrated in step iii, scheme 5).

### 1-(2-cyanoacetyl)-5-phenylpiperidine-3-carboxylic acid

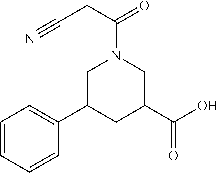 [SMILES: N#CCC(=O)N1CC(C(=O)O)CC(c2ccccc2)C1]

To a stirred solution of 5-phenylpiperidine-3-carboxylic acid (1.0 equiv, 0.600 g, 2.92 mmol in Dioxane (5.0 ml) were added DIPEA (5 equiv., 0.74 ml, 2.92 mmol) and 3-(3,5-dimethyl-1H-pyrazol-1-yl)-3-oxopropanenitrile (1.1 equiv., 0.525 g, 3.22 mmol) at RT. Reaction mixture stirred for 16 h at 90° C. The progress of the reaction was monitored by LCMS. Reaction mixture was concentrated and quenched with water (20 ml). The Aq. Layer was acidified with 1.5 N HCl and extracted with 10% MeOH: DCM (2×30 ml), Organic layer were dried over Na₂SO₄, filtered and concentrated. Crude product was purified by column chromatography (DCM/MeOH (4 to 7%)) to afford 1-(2-cyanoacetyl)-5-phenylpiperidine-3-carboxylic acid (0.230 g, 0.845 mmol, 28.9% yield) as gummy liquid. LCMS: m/z=[M−H]⁻ 271.0; ¹H-NMR (400 MHz, DMSO-d6): δ 11.37 (bs, 1H), 7.33-7.21 (m, 5H), 4.02 (s 2H), 3.17-2.79 (m, 4H), 2.70-2.62 (m, 1H), 2.192 (m, 1H), 2.084-1.96 (m, 2H).

| Example | | m/z |
|---|---|---|
| number | Structure/Preparation | (M + H)⁺ |
| 1 | 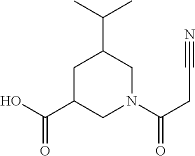 [SMILES: CC(C)C1CC(C(=O)O)CN(C(=O)CC#N)C1] | 238.1 |
| 2 | 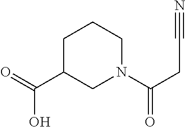 [SMILES: N#CCC(=O)N1CCCC(C(=O)O)C1] | 197.2 |
| 3 | 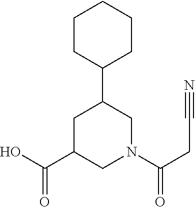 [SMILES: N#CCC(=O)N1CC(C(=O)O)CC(C2CCCCC2)C1] | 278.2 |
| 4 | 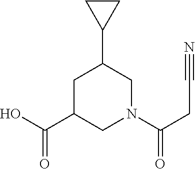 [SMILES: N#CCC(=O)N1CC(C(=O)O)CC(C2CC2)C1] | 237.2 |
| 5 | 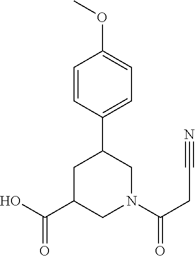 [SMILES: COc1ccc(C2CC(C(=O)O)CN(C(=O)CC#N)C2)cc1] | 303.1 |
| 6 | 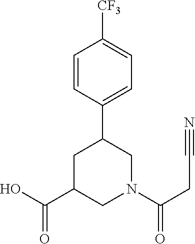 [SMILES: N#CCC(=O)N1CC(C(=O)O)CC(c2ccc(C(F)(F)F)cc2)C1] | 341.2 |
| 7 | 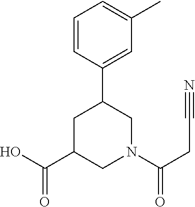 [SMILES: Cc1cccc(C2CC(C(=O)O)CN(C(=O)CC#N)C2)c1] | 287.1 |
| 8 | 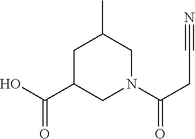 | 211.1 |

The resulting compounds are then reacted with the aldehydes shown below (as illustrated in step iv, scheme 5).

Aldehyde Y

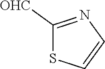

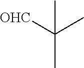

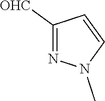

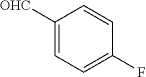

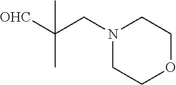

### (E/Z)-1-(2-cyano-4,4-dimethylpent-2-enoyl)-5-phenylpiperidine-3-carboxylic acid (Pipacid 0)

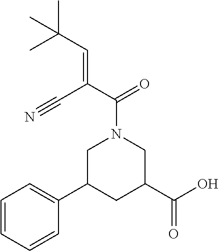 [SMILES: CC(C)(C)/C=C(\C#N)C(=O)N1CC(C(=O)O)CC(c2ccccc2)C1]

To a stirred solution of 1-(2-cyanoacetyl)-5-phenylpiperidine-3-carboxylic acid (0.070 g, 0.257 mmol in ethanol (2 ml) was added piperidine (0.044 g, 0.514 mmol) followed by addition of pivalaldehyde (0.033 g, 0.386 mmol) at RT under nitrogen atmosphere. The resulting reaction mixture was stirred at 60° C. for 16 h. Reaction was monitored by LCMS. The reaction was concentrated under reduced pressure to get a crude product. The crude product was acidified by adding 2N HCl in water and then product extracted in 10% MeOH/DCM (3×20 mL). Organic layer was dried over Na₂SO₄, filtered and concentrated under reduced pressure. The crude compound was purified by flash column chromatography (DCM/MeOH (0 to 10%)) to give (E)-1-(2-cyano-4,4-dimethylpent-2-enoyl)-5-phenylpiperidine-3-carboxylic acid (0.030 g, 0.060 mmol, 23.31% yield as a sticky oil. LCMS: m/z=[M−H]⁻¹ 1 339.4; ¹H-NMR (400 MHz, DMSO-d6): δ 12.42 (s, 1H), 7.32 (m, 6H), 3.30-3.17 (m, 4H), 2.70-2.62 (m, 1H), 2.192 (m, 1H), 2.084-1.96 (m, 2H), 1.09 (s, 9H).

| | | m/z |
|---|---|---|
| Example number | Structure/Preparation | (M + H⁾⁺¹ |
| Pipacid 1 | 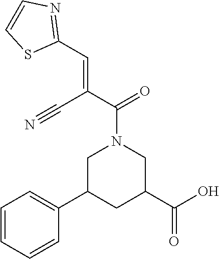 [SMILES: N#C/C(=C\c1nccs1)C(=O)N1CC(C(=O)O)CC(c2ccccc2)C1] | 368.1 |
| Pipacid 2 | 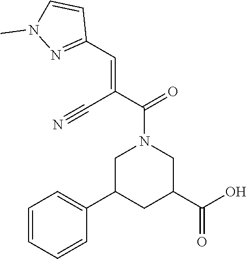 [SMILES: Cn1ccc(/C=C(\C#N)C(=O)N2CC(C(=O)O)CC(c3ccccc3)C2)n1] | 365.3 |
| Pipacid 3 | 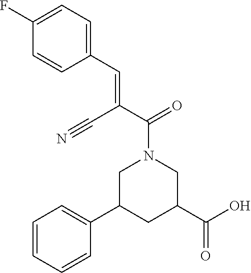 [SMILES: N#C/C(=C\c1ccc(F)cc1)C(=O)N1CC(C(=O)O)CC(c2ccccc2)C1] | 379.2 |
| Pipacid 4 | 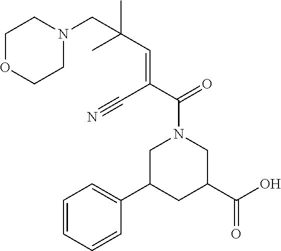 [SMILES: CC(C)(/C=C(\C#N)C(=O)N1CC(C(=O)O)CC(c2ccccc2)C1)CN1CCOCC1] | 426.2 |
| Pipacid 5 | 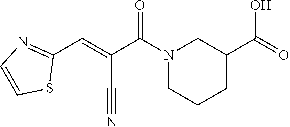 [SMILES: N#C/C(=C\c1nccs1)C(=O)N1CCCC(C(=O)O)C1] | 293.2 |
| Pipacid 6 | 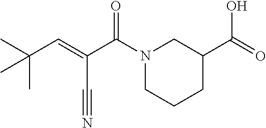 [SMILES: CC(C)(C)/C=C(\C#N)C(=O)N1CCCC(C(=O)O)C1] | 265.1 |
| Pipacid 7 | 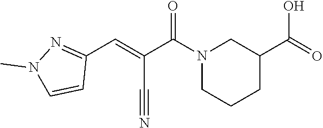 [SMILES: Cn1ccc(/C=C(\C#N)C(=O)N2CCCC(C(=O)O)C2)n1] | 289.3 |
| Pipacid 8 | 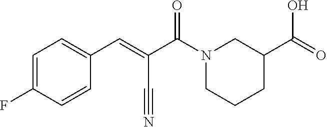 [SMILES: N#C/C(=C\c1ccc(F)cc1)C(=O)N1CCCC(C(=O)O)C1] | 303.1 |
| Pipacid 9 | 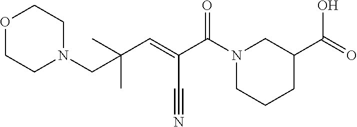 [SMILES: CC(C)(/C=C(\C#N)C(=O)N1CCCC(C(=O)O)C1)CN1CCOCC1] | 350.1 |
| Pipacid 10 | 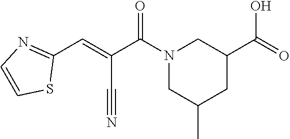 [SMILES: CC1CC(C(=O)O)CN(C(=O)/C(C#N)=C/c2nccs2)C1] | 306.2 |
| Pipacid 11 | 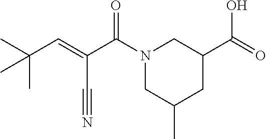 [SMILES: CC1CC(C(=O)O)CN(C(=O)/C(C#N)=C/C(C)(C)C)C1] | 279.3 |
| Pipacid 12 | 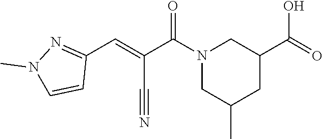 [SMILES: CC1CC(C(=O)O)CN(C(=O)/C(C#N)=C/c2ccn(C)n2)C1] | 303.3 |
| Pipacid 13 | 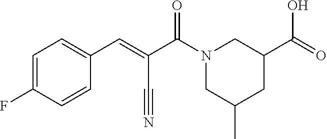 [SMILES: CC1CC(C(=O)O)CN(C(=O)/C(C#N)=C/c2ccc(F)cc2)C1] | 317.1 |
| Pipacid 14 | 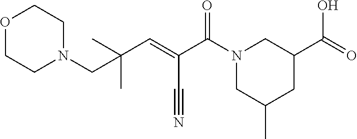 [SMILES: CC1CC(C(=O)O)CN(C(=O)/C(C#N)=C/C(C)(C)CN2CCOCC2)C1] | 364.2 |
| Pipacid 15 | 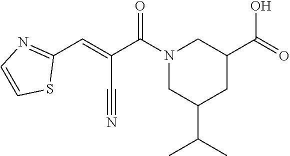 [SMILES: CC(C)C1CC(C(=O)O)CN(C(=O)/C(C#N)=C/c2nccs2)C1] | 334.1 |
| Pipacid 16 | 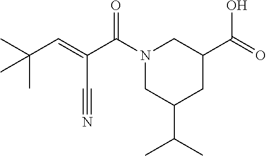 [SMILES: CC(C)C1CC(C(=O)O)CN(C(=O)/C(C#N)=C/C(C)(C)C)C1] | 307.1 |
| Pipacid 17 | 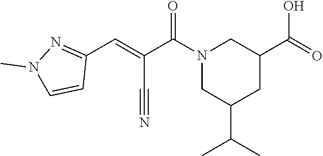 [SMILES: CC(C)C1CC(C(=O)O)CN(C(=O)/C(C#N)=C/c2ccn(C)n2)C1] | 331.3 |
| Pipacid 18 | 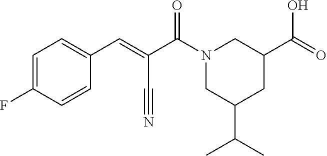 [SMILES: CC(C)C1CC(C(=O)O)CN(C(=O)/C(C#N)=C/c2ccc(F)cc2)C1] | 345.1 |
| Pipacid 19 | 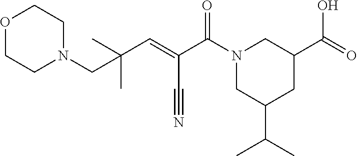 [SMILES: CC(C)C1CC(C(=O)O)CN(C(=O)/C(C#N)=C/C(C)(C)CN2CCOCC2)C1] | 392.2 |
| Pipacid 20 | 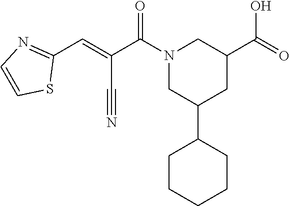 [SMILES: N#C/C(=C\c1nccs1)C(=O)N1CC(C(=O)O)CC(C2CCCCC2)C1] | 373.1 |
| Pipacid 21 | 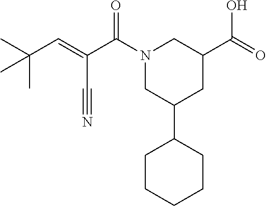 [SMILES: CC(C)(C)/C=C(\C#N)C(=O)N1CC(C(=O)O)CC(C2CCCCC2)C1] | 347.2 |
| Pipacid 22 | 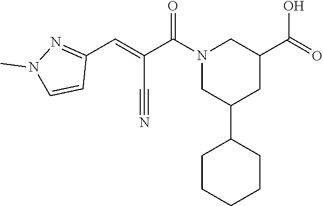 [SMILES: Cn1ccc(/C=C(\C#N)C(=O)N2CC(C(=O)O)CC(C3CCCCC3)C2)n1] | 371.1 |
| Pipacid 23 | 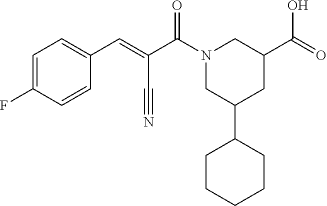 [SMILES: N#C/C(=C\c1ccc(F)cc1)C(=O)N1CC(C(=O)O)CC(C2CCCCC2)C1] | 385.1 |
| Pipacid 24 | 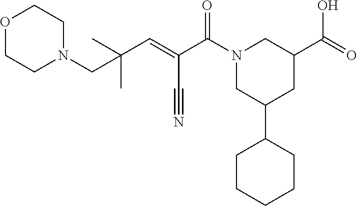 [SMILES: CC(C)(/C=C(\C#N)C(=O)N1CC(C(=O)O)CC(C2CCCCC2)C1)CN1CCOCC1] | 432.2 |
| Pipacid 25 | 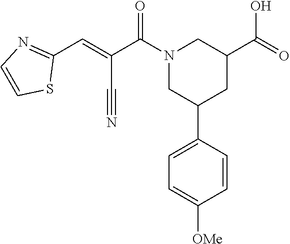 [SMILES: COc1ccc(C2CC(C(=O)O)CN(C(=O)/C(C#N)=C/c3nccs3)C2)cc1] | 398.2 |
| Pipacid 26 | 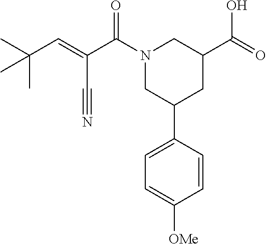 [SMILES: COc1ccc(C2CC(C(=O)O)CN(C(=O)/C(C#N)=C/C(C)(C)C)C2)cc1] | 371.3 |
| Pipacid 27 | 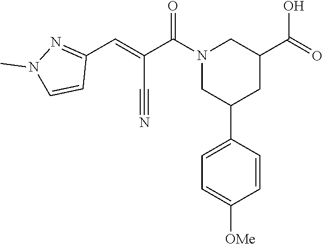 [SMILES: COc1ccc(C2CC(C(=O)O)CN(C(=O)/C(C#N)=C/c3ccn(C)n3)C2)cc1] | 395.2 |
| Pipacid 28 | 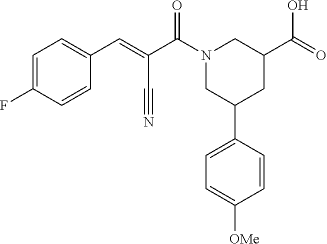 [SMILES: COc1ccc(C2CC(C(=O)O)CN(C(=O)/C(C#N)=C/c3ccc(F)cc3)C2)cc1] | 409.1 |
| Pipacid 29 | 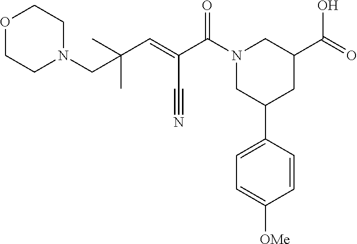 [SMILES: COc1ccc(C2CC(C(=O)O)CN(C(=O)/C(C#N)=C/C(C)(C)CN3CCOCC3)C2)cc1] | 456.2 |
| Pipacid 30 | 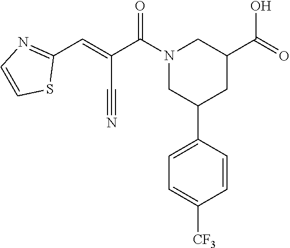 [SMILES: N#C/C(=C\c1nccs1)C(=O)N1CC(C(=O)O)CC(c2ccc(C(F)(F)F)cc2)C1] | 436.2 |
| Pipacid 31 | 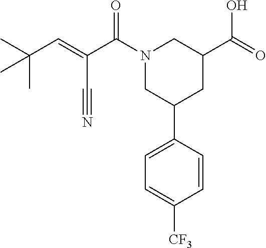 [SMILES: CC(C)(C)/C=C(\C#N)C(=O)N1CC(C(=O)O)CC(c2ccc(C(F)(F)F)cc2)C1] | 410.1 |
| Pipacid 32 | 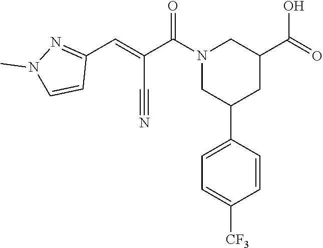 [SMILES: Cn1ccc(/C=C(\C#N)C(=O)N2CC(C(=O)O)CC(c3ccc(C(F)(F)F)cc3)C2)n1] | 433.3 |
| Pipacid 33 | 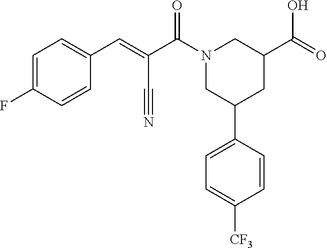 [SMILES: N#C/C(=C\c1ccc(F)cc1)C(=O)N1CC(C(=O)O)CC(c2ccc(C(F)(F)F)cc2)C1] | 447.2 |
| Pipacid 34 | 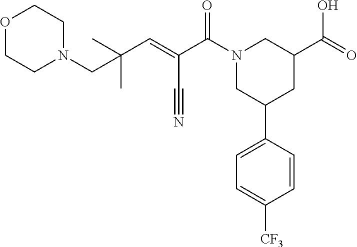 [SMILES: CC(C)(/C=C(\C#N)C(=O)N1CC(C(=O)O)CC(c2ccc(C(F)(F)F)cc2)C1)CN1CCOCC1] | 494.2 |
| Pipacid 35 | 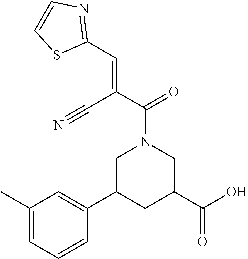 [SMILES: Cc1cccc(C2CC(C(=O)O)CN(C(=O)/C(C#N)=C/c3nccs3)C2)c1] | 382.1 |
| Pipacid 36 | 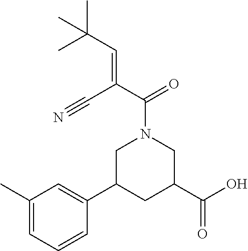 [SMILES: Cc1cccc(C2CC(C(=O)O)CN(C(=O)/C(C#N)=C/C(C)(C)C)C2)c1] | 355.1 |
| Pipacid 37 | 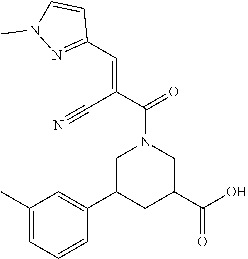 [SMILES: Cc1cccc(C2CC(C(=O)O)CN(C(=O)/C(C#N)=C/c3ccn(C)n3)C2)c1] | 379.1 |
| Pipacid | 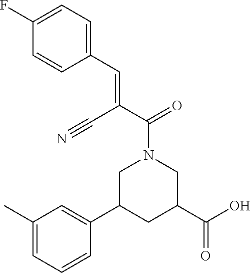 [SMILES: Cc1cccc(C2CC(C(=O)O)CN(C(=O)/C(C#N)=C/c3ccc(F)cc3)C2)c1] | 393.2 |
| Pipacid 39 | 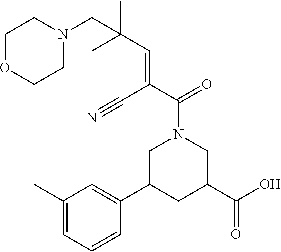 [SMILES: Cc1cccc(C2CC(C(=O)O)CN(C(=O)/C(C#N)=C/C(C)(C)CN3CCOCC3)C2)c1] | 440.2 |

### Synthesis of the Final Bifunctional Degraders is Illustrated in Scheme 6

Piperidine-acrylamide acids and amine-linker functionalised target protein binding ligand are dissolved in DMF and treated with HATU and DIPEA at room temperature to afford the bifunctional compounds.

Example compounds that are made in accordance with the method illustrated in scheme 6 are shown below.

### N-(1-(4-((2S,4R)-1-acetyl-4-((4-chlorophenyl)amino)-2-methyl-1,2,3,4-tetrahydroquinolin-6-yl)phenyl)-1-oxo-5,8, 11, 14, 17-pentaoxa-2-azanonadecan-19-yl)-1-((E)-2-cyano-3-(thiazol-2-yl)acryloyl)-5-phenylpiperidine-3-carboxamide

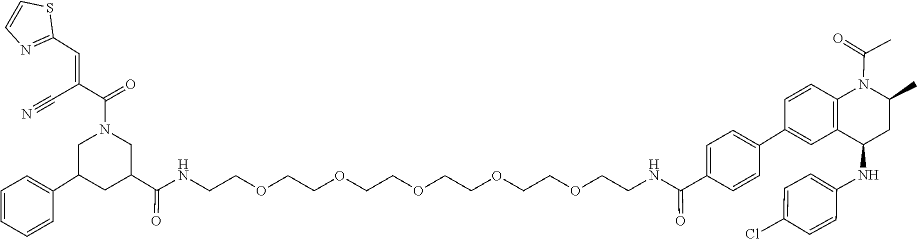 [SMILES: CC(=O)N1c2ccc(-c3ccc(C(=O)NCCOCCOCCOCCOCCOCCNC(=O)C4CC(c5ccccc5)CN(C(=O)/C(C#N)=C/c5nccs5)C4)cc3)cc2[C@H](Nc2ccc(Cl)cc2)C[C@@H]1C]

To a stirred solution of 4-((2S,4R)-1-acetyl-4-((4-chlorophenyl)amino)-2-methyl-1,2,3,4-tetrahydroquinolin-6-yl)-N-(17-amino-3,6,9, 12, 15-pentaoxaheptadecyl)benzamide hydrochloride (0.010 g, 0.014 mmol) and (E)-1-(2-cyano-3-(thiazol-2-yl) acryloyl)-5-phenylpiperidine-3-carboxylic acid (6.01 mg, 0.016 mmol) in DMF (1 mL) was added DIPEA (5.28 mg, 0.041 mmol) followed by addition of HATU (6.22 mg, 0.016 mmol) at RT under nitrogen atmosphere. The resulting reaction mixture was stirred at RT for 16 h. The reaction mixture was diluted with water and product was extracted ethyl acetate (20 ml×2). The combined organic extracts were dried over sodium sulphate, filtered, and concentrated under reduced pressure. The crude residue was purified by mass directed preparative HPLC (Mobile phase A: 0.1% HCOOH in H₂O, Mobile phase B: 0.1% HCOOH in ACN, Column: X-select C18 250*19.0 5 u). After purification, the desired fractions were lyophilized to give N-(1-(4-((2S,4R)-1-acetyl-4-((4-chlorophenyl)amino)-2-methyl-1,2,3,4-tetrahydroquinolin-6-yl)phenyl)-1-oxo-5,8,11,14,17-pentaoxa-2-azanonadecan-19-yl)-1-((E)-2-cyano-3-(thiazol-2-yl) acryloyl)-5-phenylpiperidine-3-carboxamide (5.40 mg, 5.11 mmol, 38% yield) as a colourless solid. LCMS: m/z=[M+H]⁺ 1046.2 and [M+H+2]⁺ 1048.2 (Cl-pattern); ¹H NMR (400 MHz, DMSO-d₆) 1.10 (d, J=6.3 Hz, 3H), 1.15-1.30 (m, 2H), 1.88-2.09 (m, 2H), 2.14 (s, 3H), 2.62 (td, J=8.4, 4.4 Hz, 2H), 2.68 (dt, J=3.7, 1.9 Hz, 2H), 3.14-3.26 (m, 4H), 3.35-3.60 (m, 24H), 4.31 (ddd, J=11.7, 7.8, 3.9 Hz, 1H), 4.66-4.78 (m, 1H), 6.29 (d, J=8.1 Hz, 1H), 6.72-6.79 (m, 2H), 7.09-7.16 (m, 2H), 7.20-7.39 (m, 5H), 7.42-7.48 (m, 2H), 7.59-7.65 (m, 3H), 7.90 (d, J=8.5 Hz, 2H), 8.02 (s, 1H), 8.12-8.22 (m, 2H), 8.51 (t, J=5.4 Hz, 1H).

### Further Examples

| Example | | m/z |
|---|---|---|
| No. | Structure | [M + H]⁺ |
| 13a | 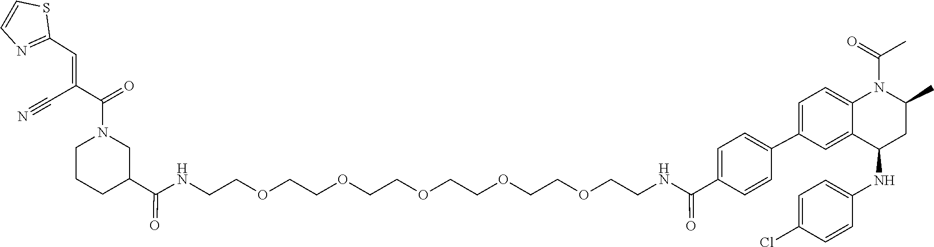 [SMILES: CC(=O)N1c2ccc(-c3ccc(C(=O)NCCOCCOCCOCCOCCOCCNC(=O)C4CCCN(C(=O)/C(C#N)=C/c5nccs5)C4)cc3)cc2[C@H](Nc2ccc(Cl)cc2)C[C@@H]1C] | [M]⁺ 1019.2 and [M + 2]⁺ 1021.2 |
| 13b | 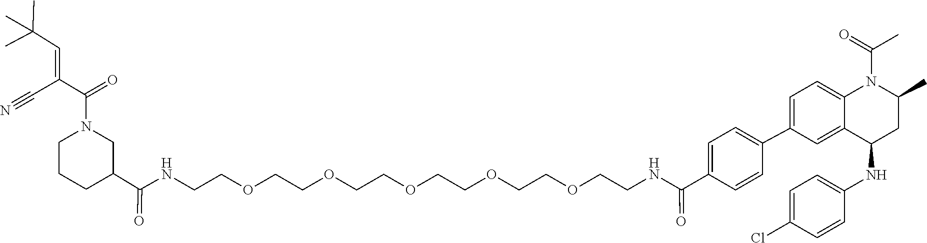 [SMILES: CC(=O)N1c2ccc(-c3ccc(C(=O)NCCOCCOCCOCCOCCOCCNC(=O)C4CCCN(C(=O)/C(C#N)=C/C(C)(C)C)C4)cc3)cc2[C@H](Nc2ccc(Cl)cc2)C[C@@H]1C] | [M]⁺ 943.2 and [M + 2]⁺ 945.2 |
| 13c | 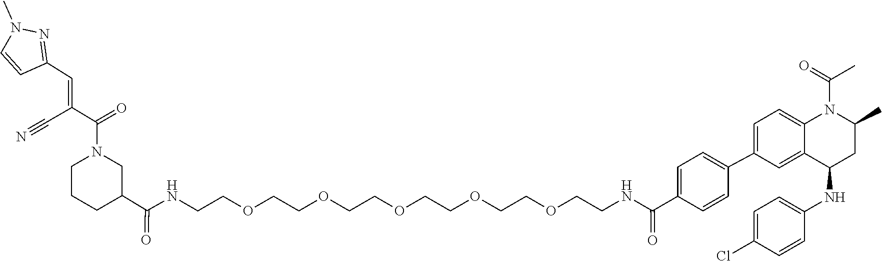 [SMILES: CC(=O)N1c2ccc(-c3ccc(C(=O)NCCOCCOCCOCCOCCOCCNC(=O)C4CCCN(C(=O)/C(C#N)=C/c5ccn(C)n5)C4)cc3)cc2[C@H](Nc2ccc(Cl)cc2)C[C@@H]1C] | [M]⁺ 967.2 and [M + 2]⁺ 969.2 |
| 13d | 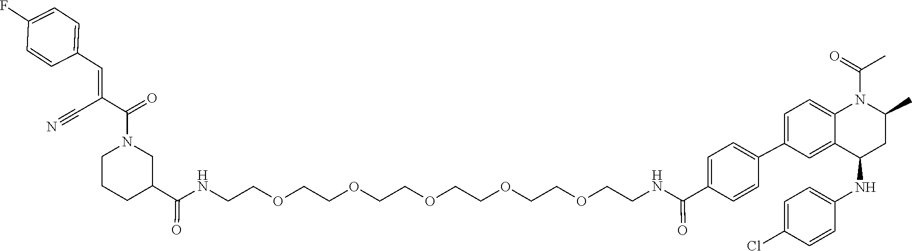 [SMILES: CC(=O)N1c2ccc(-c3ccc(C(=O)NCCOCCOCCOCCOCCOCCNC(=O)C4CCCN(C(=O)/C(C#N)=C/c5ccc(F)cc5)C4)cc3)cc2[C@H](Nc2ccc(Cl)cc2)C[C@@H]1C] | [M]⁺ 981.2 and [M + 2]⁺ 983.2 |
| 13e | 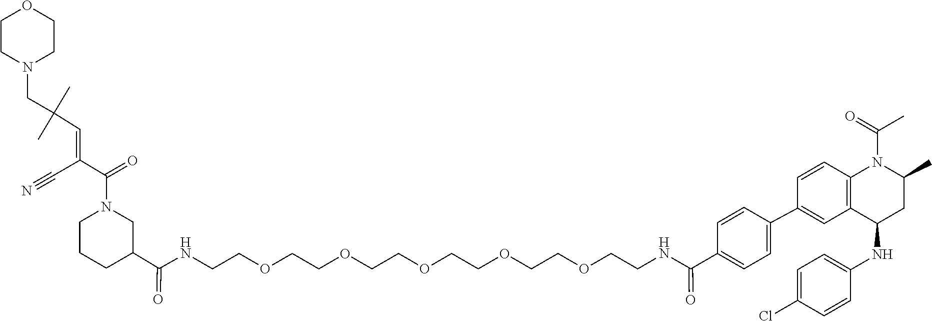 [SMILES: CC(=O)N1c2ccc(-c3ccc(C(=O)NCCOCCOCCOCCOCCOCCNC(=O)C4CCCN(C(=O)/C(C#N)=C/C(C)(C)CN5CCOCC5)C4)cc3)cc2[C@H](Nc2ccc(Cl)cc2)C[C@@H]1C] | [M]⁺ 1028.2 and [M + 2]⁺ 1030.2 |
| 13f | 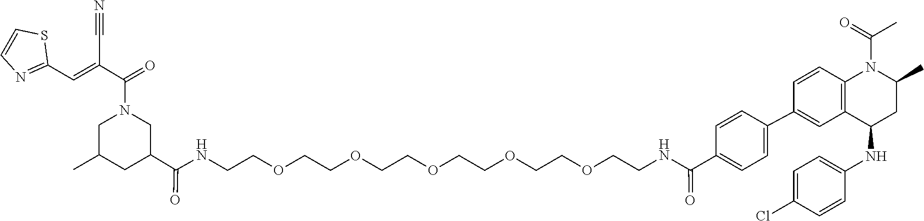 [SMILES: CC(=O)N1c2ccc(-c3ccc(C(=O)NCCOCCOCCOCCOCCOCCNC(=O)C4CC(C)CN(C(=O)/C(C#N)=C/c5nccs5)C4)cc3)cc2[C@H](Nc2ccc(Cl)cc2)C[C@@H]1C] | [M]⁺ 957.2 and [M + 2]⁺ 959.2 |
| 13g | 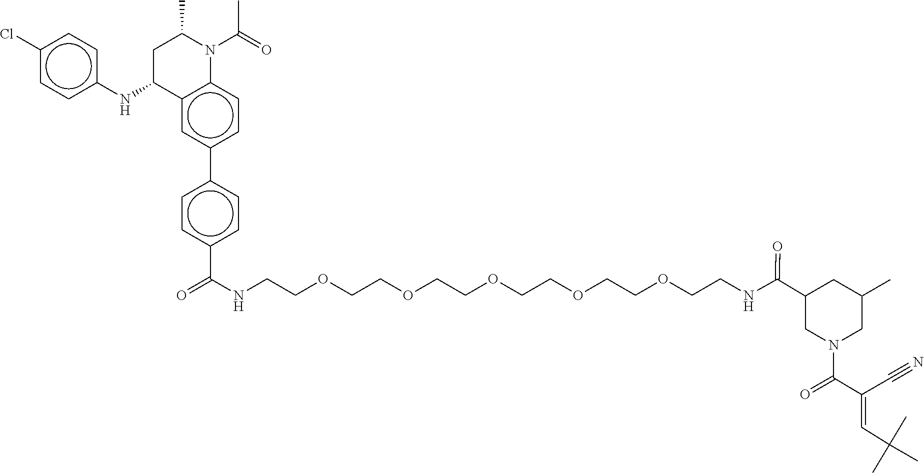 | [M]⁺ 957.2 and [M + 2]⁺ 959.2 |
| 13h | 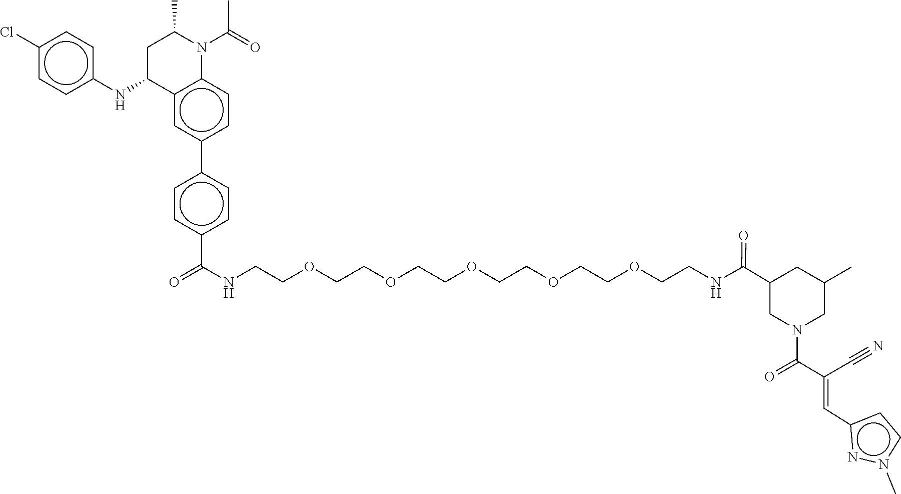 | |
| 13i | 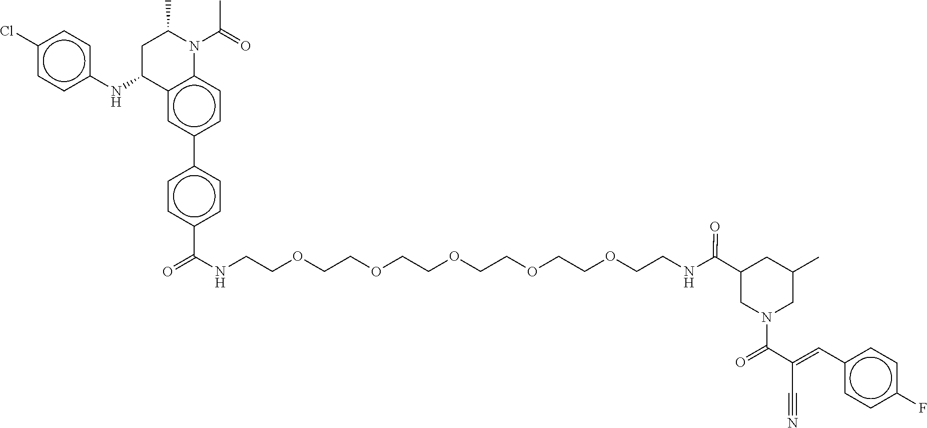 | [M]⁺ 995.2 and [M + 2]⁺ 997.2 |
| 13j | 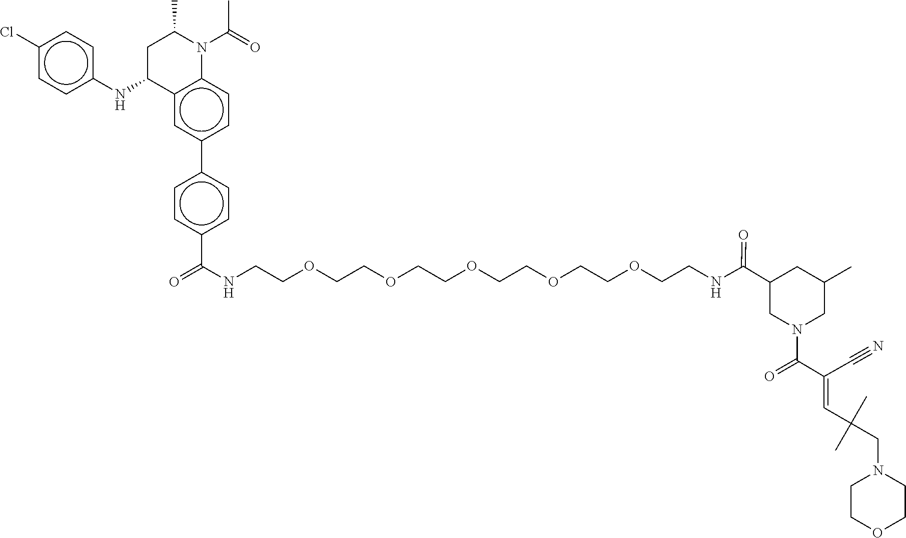 | |
| 13k | 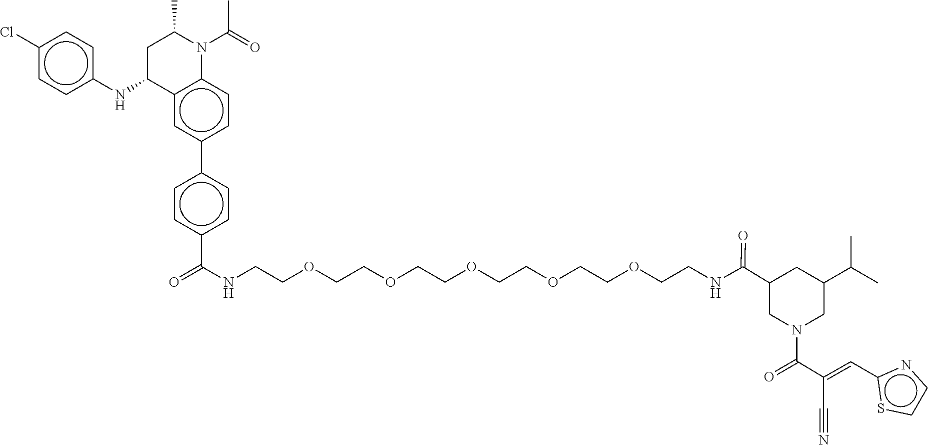 | [M]⁺ 1012.2 and [M + 2]⁺ 1014.2 |
| 13l | 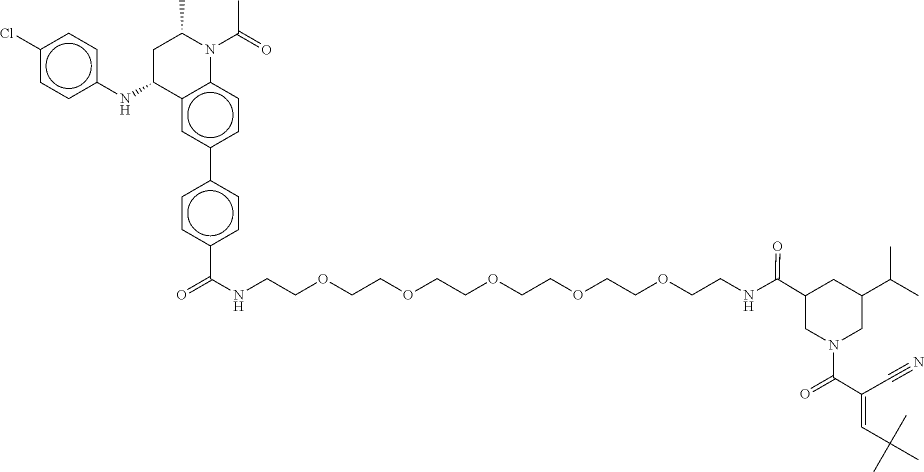 | [M]⁺ 985.2 and [M + 2]⁺ 987.2 |
| 13m | 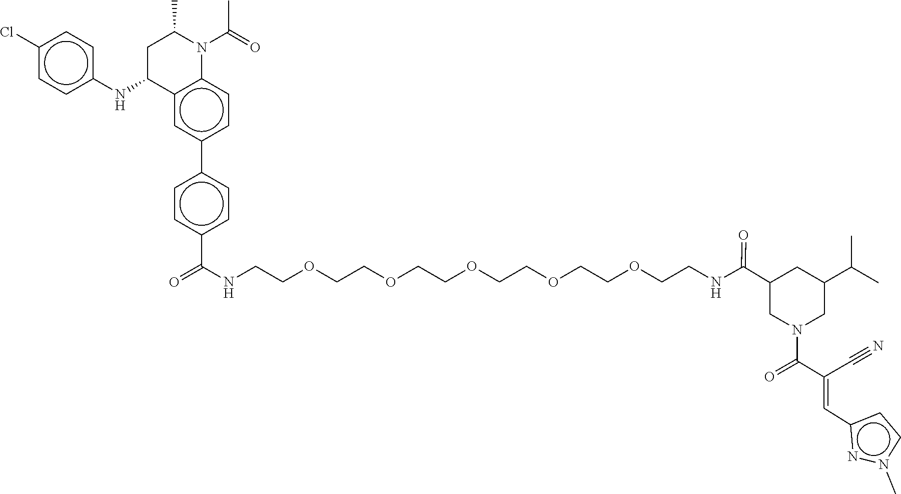 | [M]⁺ 1009.2 and [M + 2]⁺ 1011.2 |
| 13n | 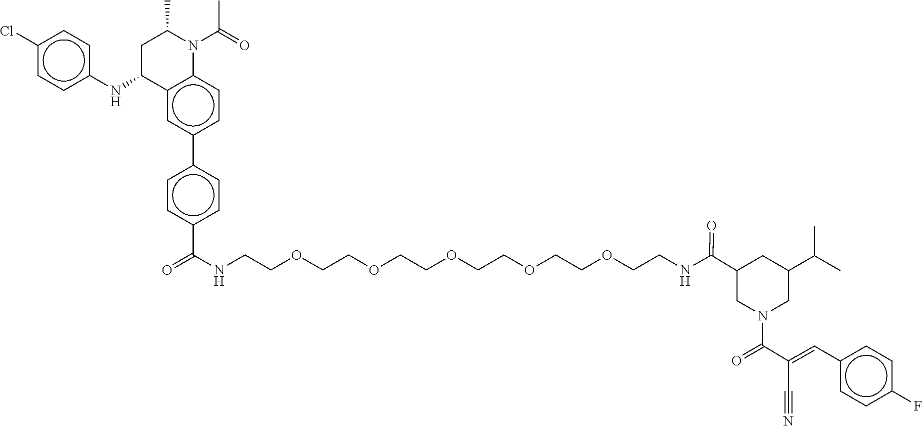 | [M]⁺ 1023.2 and [M + 2]⁺ 1025.2 |
| 13o | 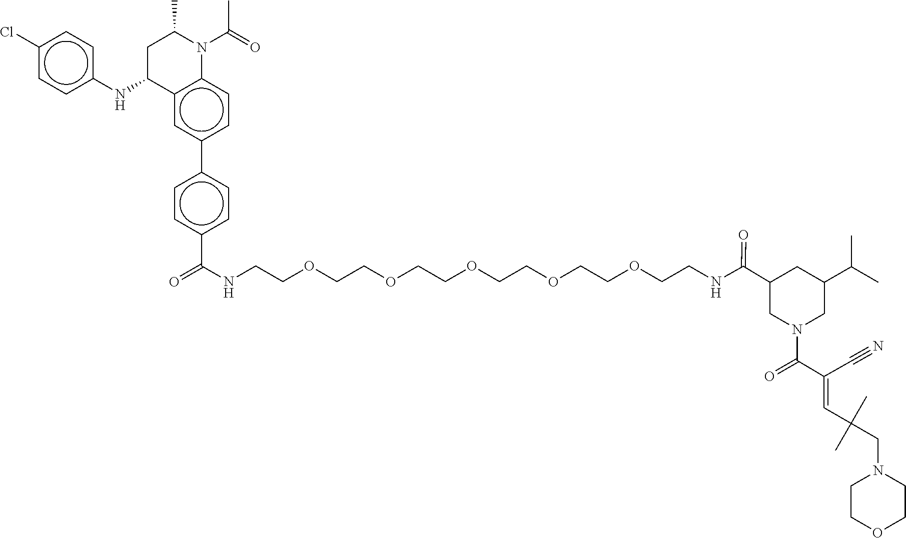 | [M]⁺ 1070.2 and [M + 2]⁺ 1072.2 |
| 13p | 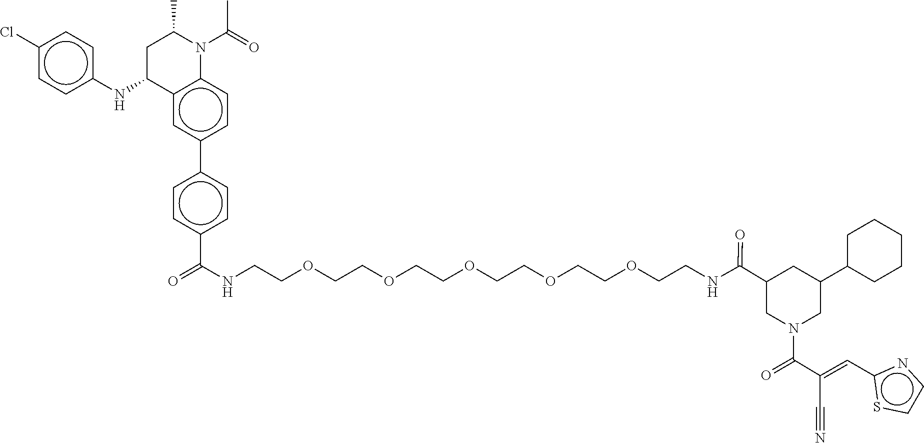 | [M]⁺ 1052.2 and [M + 2]⁺ 1054.2 |
| 13q | 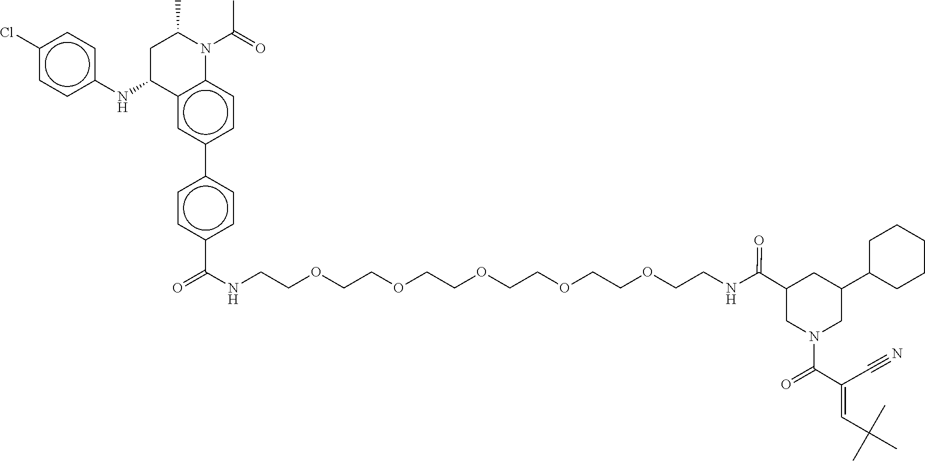 | [M]⁺ 1025.2 and [M + 2]⁺ 1027.2 |
| 13r | 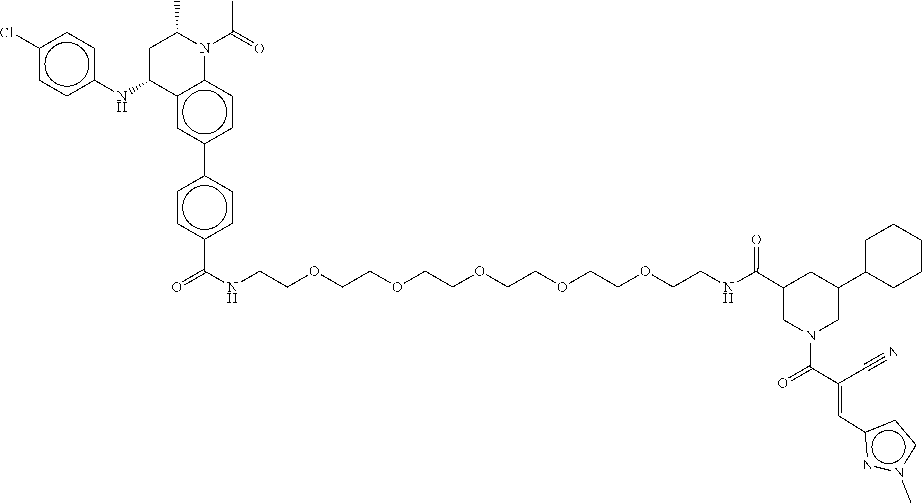 | [M]⁺ 1049.2 and [M + 2]⁺ 1051.2 |
| 13s | 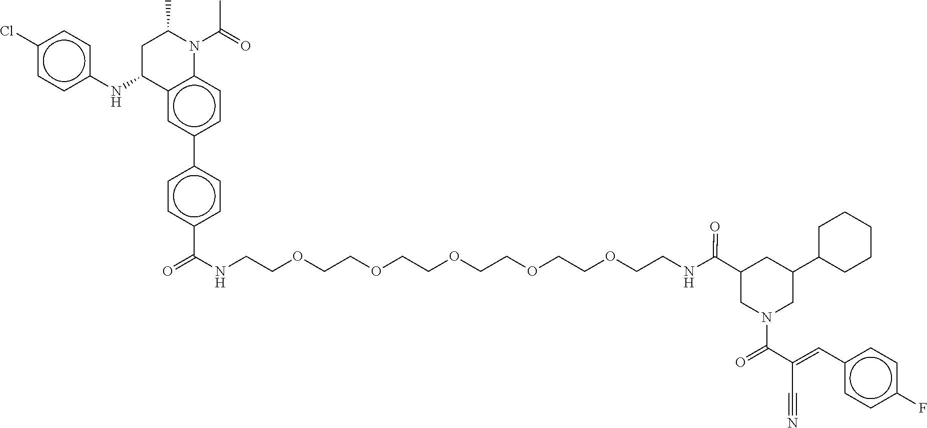 | [M]⁺ 1063.2 and [M + 2]⁺ 1065.2 |
| 13t | 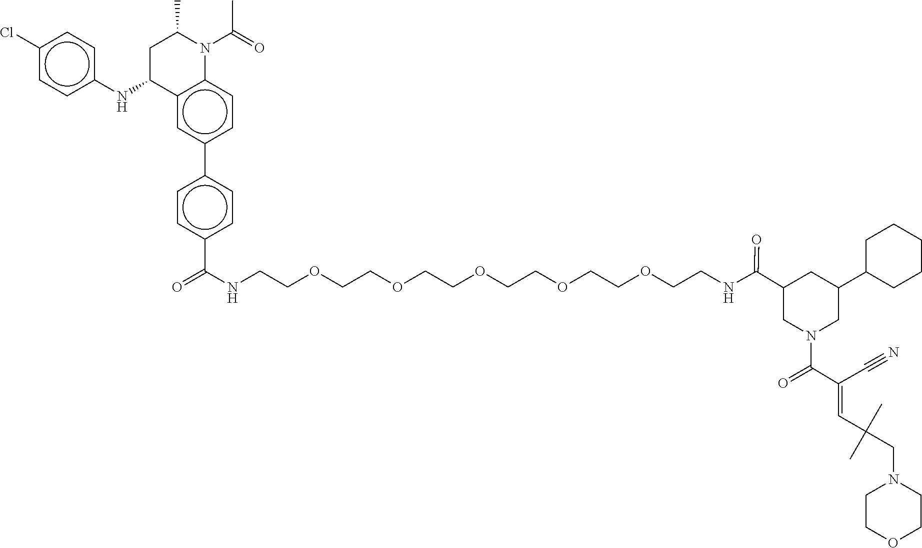 | [M]⁺ 1110.2 and [M + 2]⁺ 1112.2 |
| 13u | 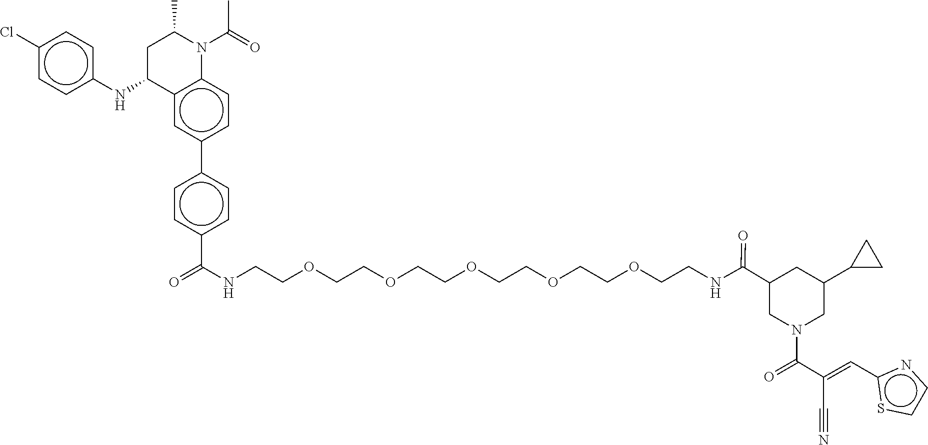 | [M]⁺ 1010.2 and [M + 2]⁺ 1012.2 |
| 13v | 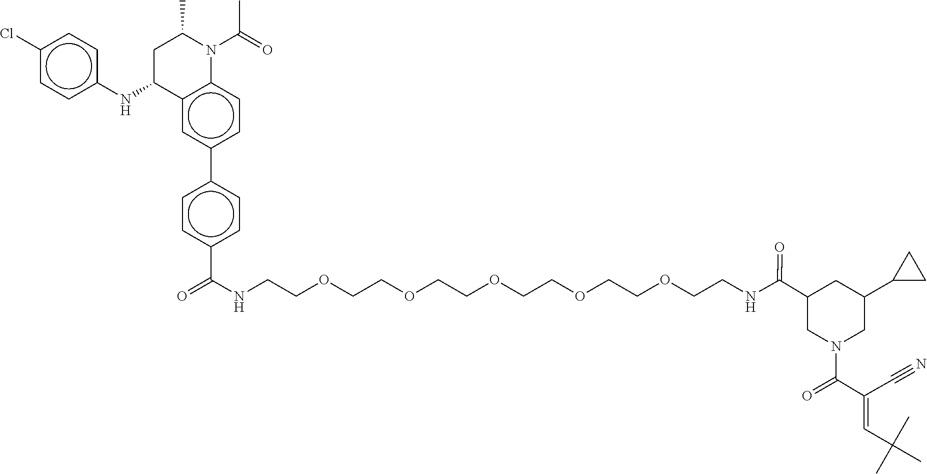 | [M]⁺ 983.2 and [M + 2]⁺ 985.2 |
| 13w | 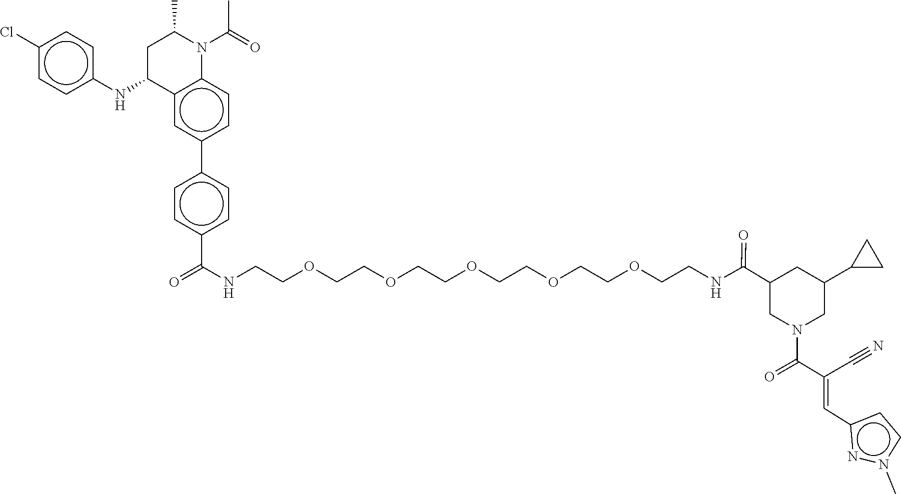 | |
| 13x | 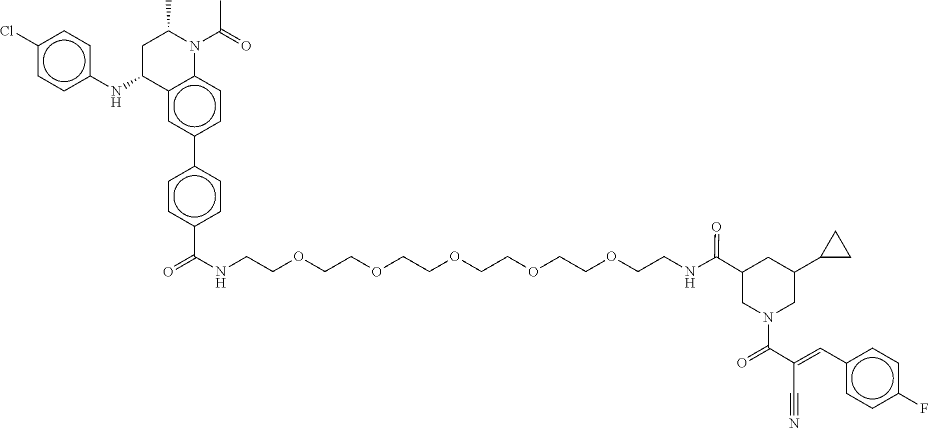 | [M]⁺ 1021.2 and [M + 2]⁺ 1023.2 |
| 13y | 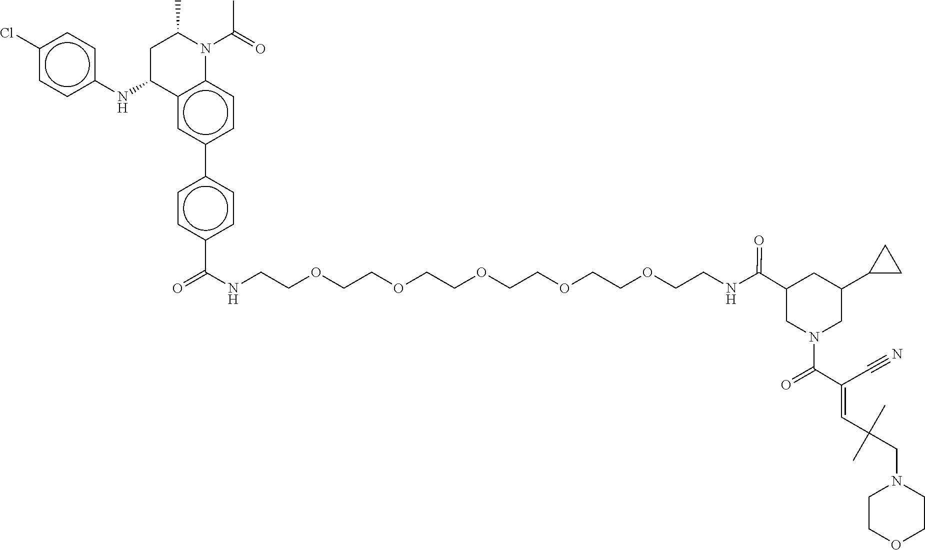 | |
| 13z | 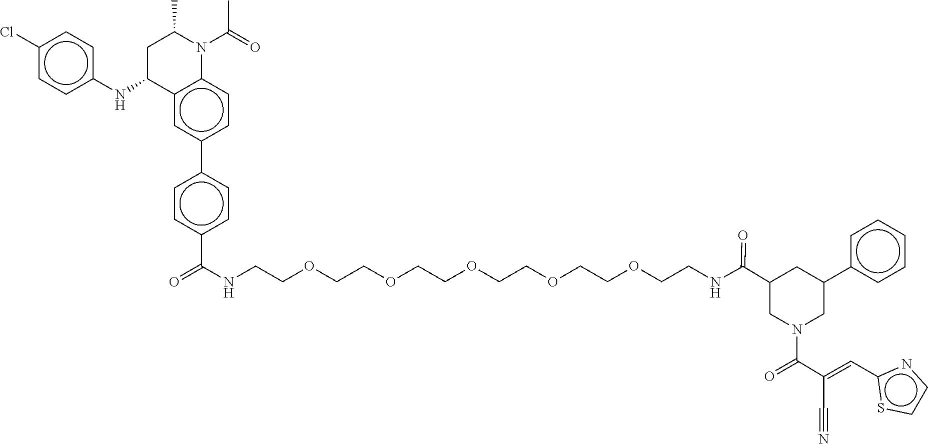 | |
| 13aa | 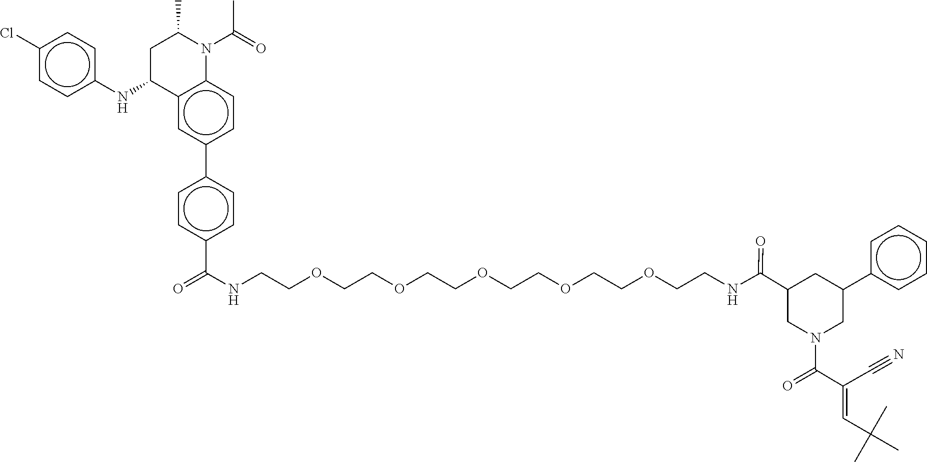 | [M]⁺ 1019.2 and [M + 2]⁺ 1021.2 |
| 13ab | 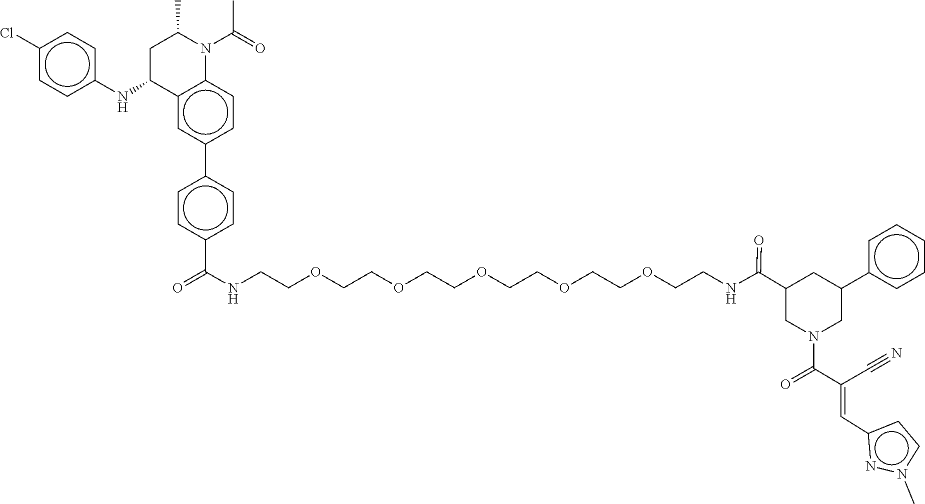 | [M]⁺ 1043.2 and [M + 2]⁺ 1045.2 |
| 13ac | 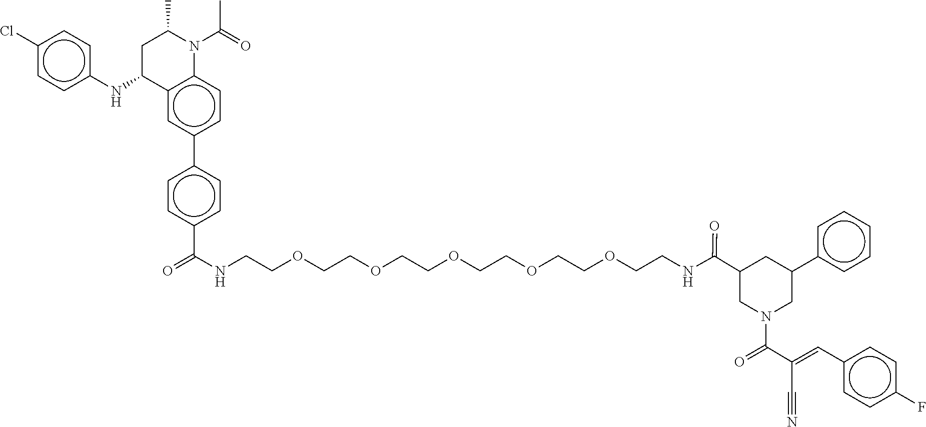 | [M]⁺ 1057.1 and [M + 2]⁺ 1059.1 |
| 13ad | 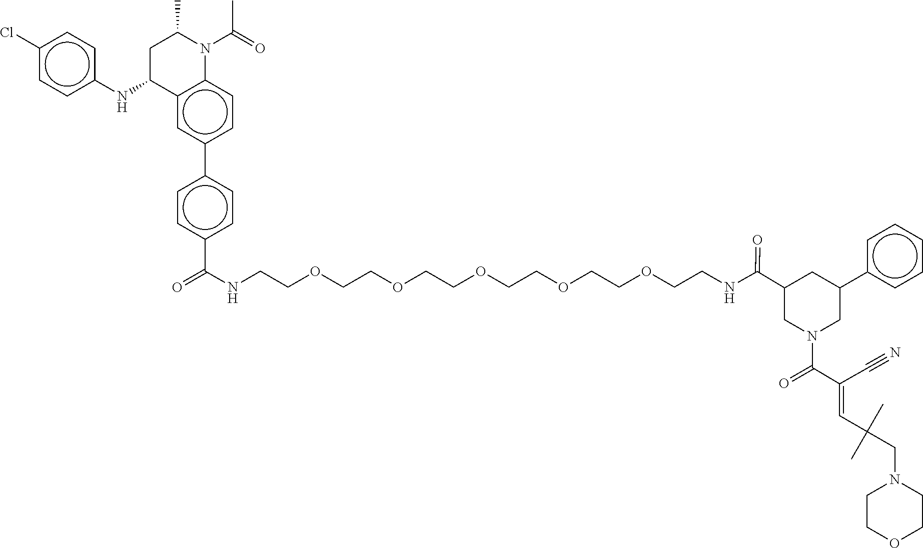 | |
| 13ae | 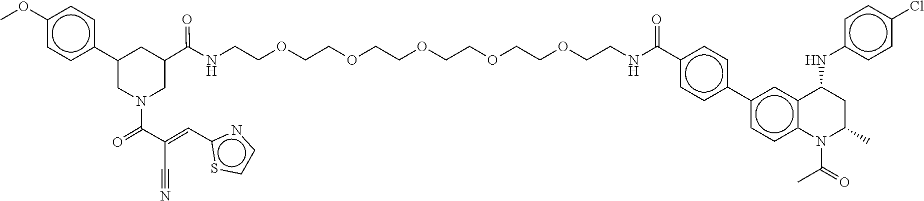 | [M]⁺ 1076.2 and [M + 2]⁺ 1078.2 |
| 13af | 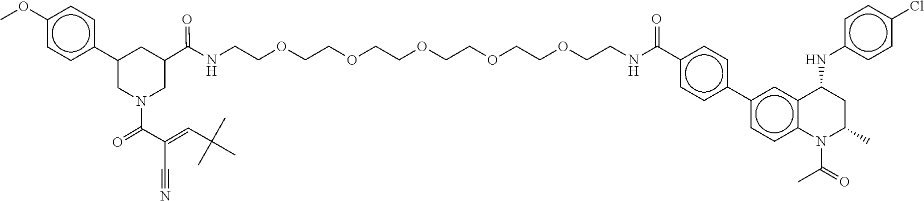 | [M]⁺ 1049.2 and [M + 2]⁺ 1051.2 |
| 13ag | 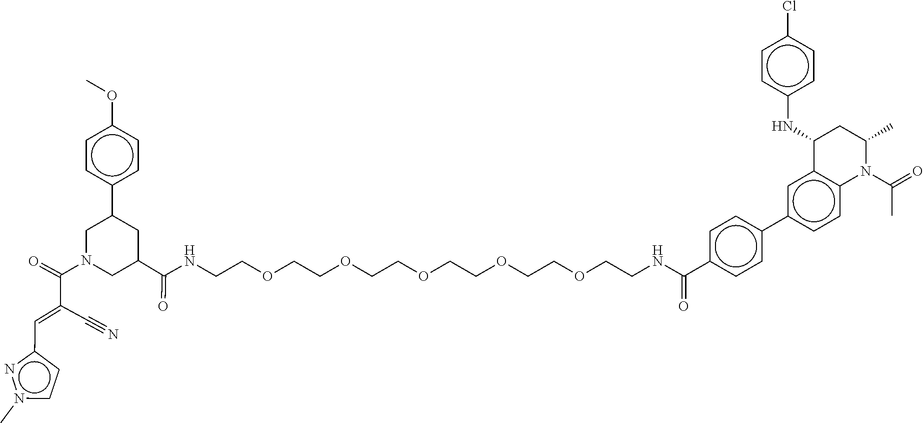 | [M]⁺ 1073.2 and [M + 2]⁺ 1075.2 |
| 13ah | 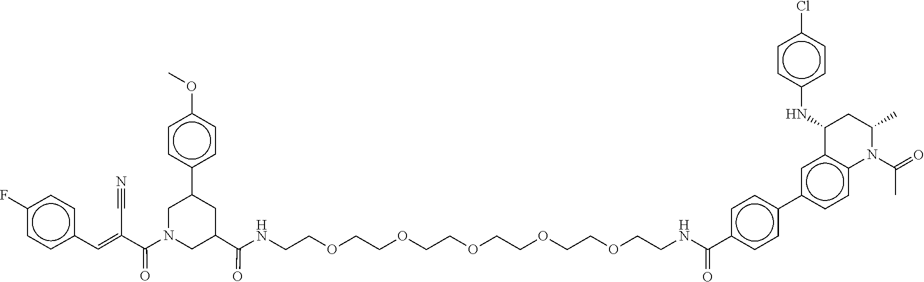 | [M]⁺ 1087.2 and [M + 2]⁺ 1089.2 |
| 13ai | 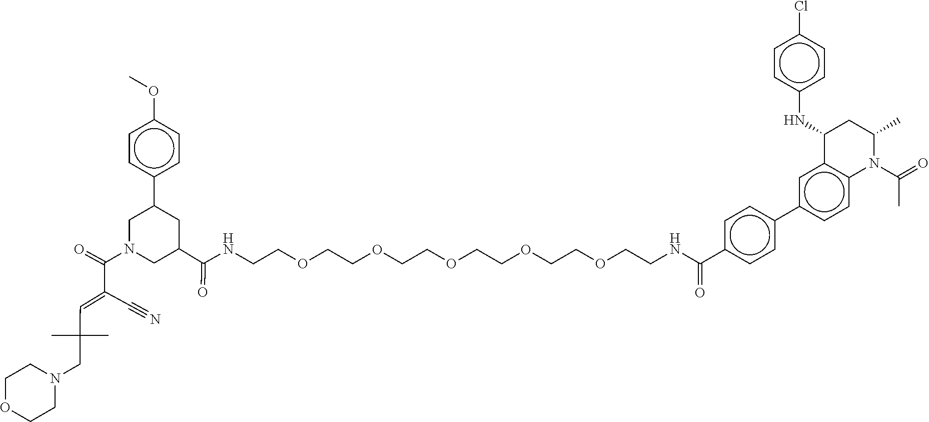 | [M]⁺ 1134.2 and [M + 2]⁺ 1136.2 |
| 13aj | 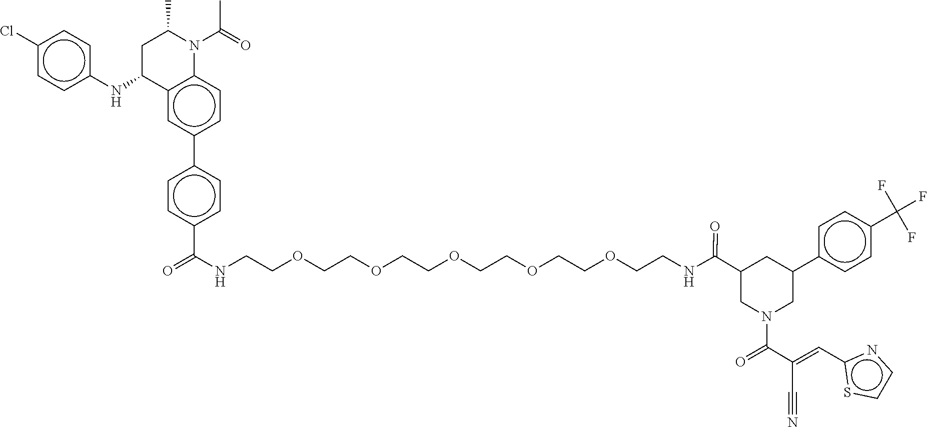 | [M]⁺ 1114.2 and [M + 2]⁺ 1116.2 |
| 13ak | 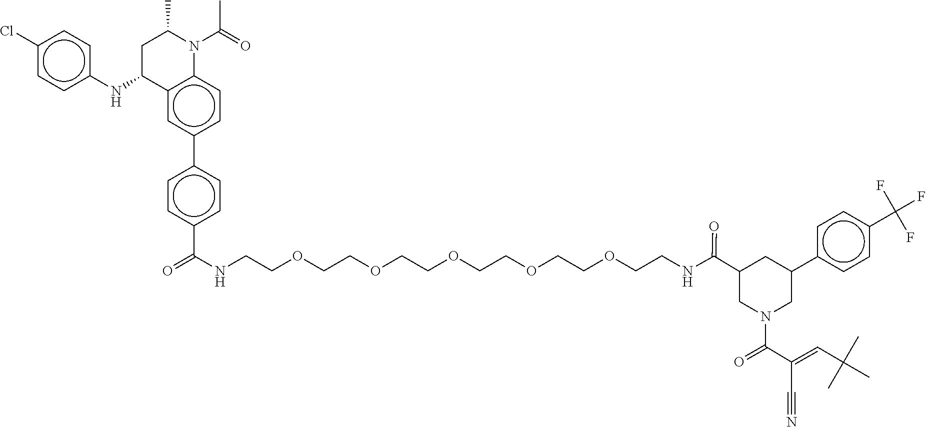 | [M]⁺ 1087.2 and [M + 2]⁺ 1089.2 |
| 13al | 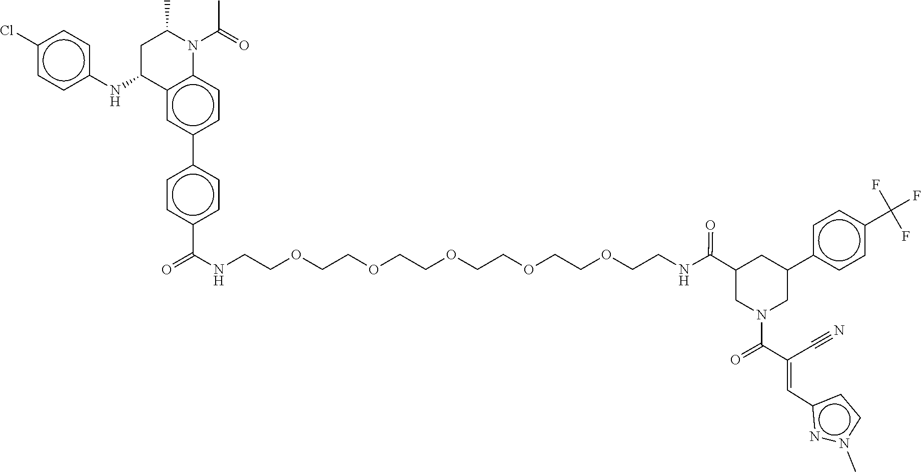 | [M]⁺ 1110.2 and [M + 2]⁺ 1112.2 |
| 13am | 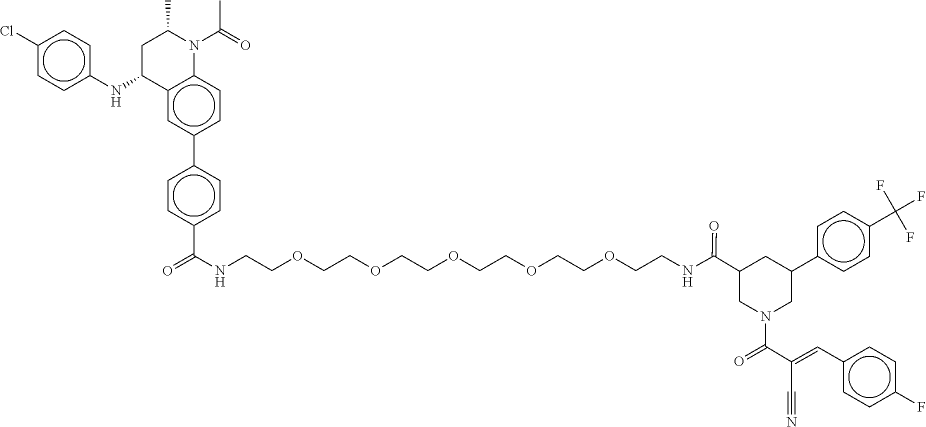 | [M]⁺ 1125.2 and [M + 2]⁺ 1127.2 |
| 13an | 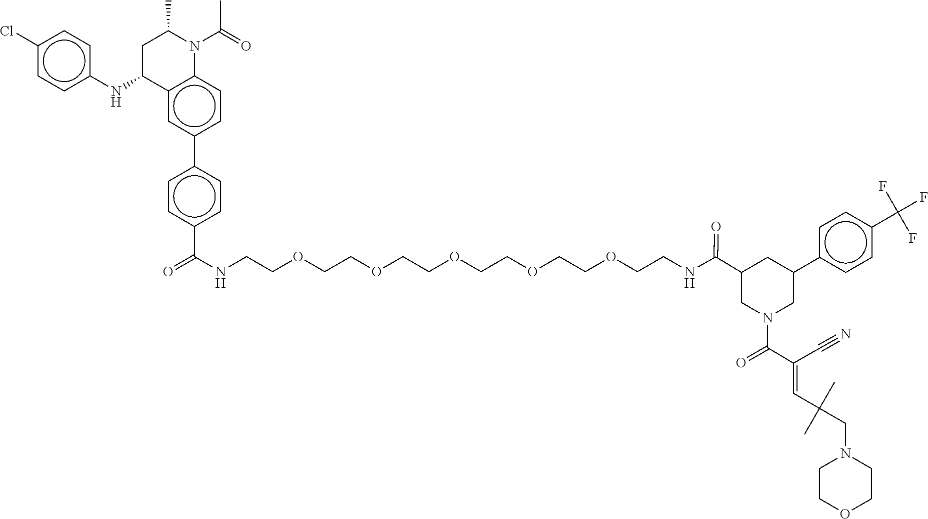 | [M]⁺ 1172.2 and [M + 2]⁺ 1174.2 |
| 13ao | 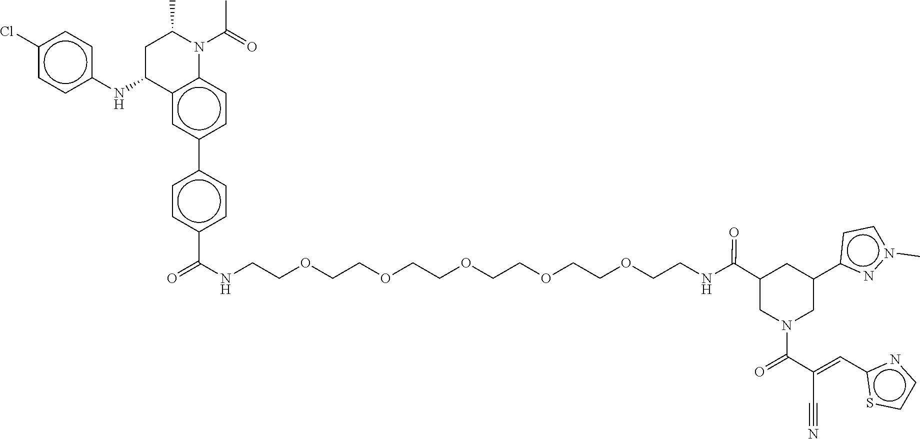 | |
| 13ap | 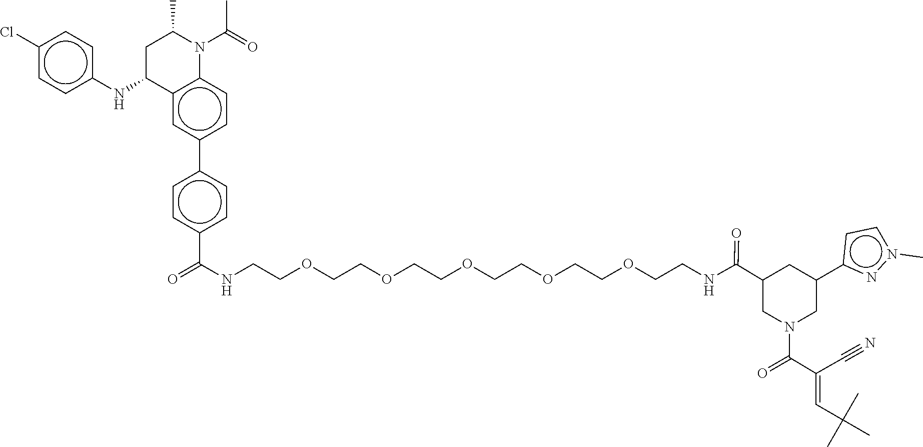 | |
| 13aq | 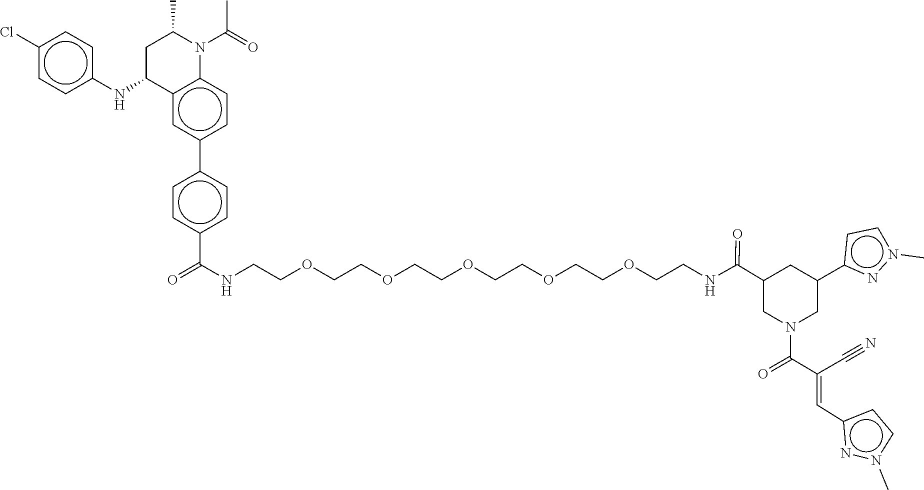 | |
| 13ar | 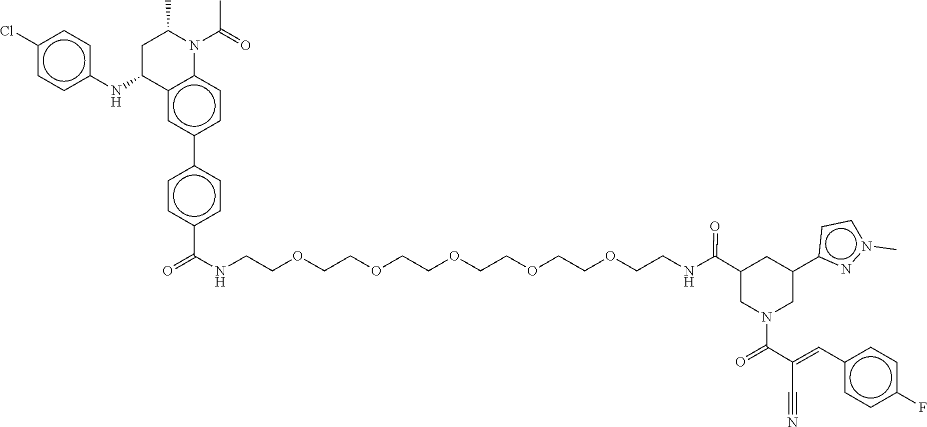 | |
| 13as | 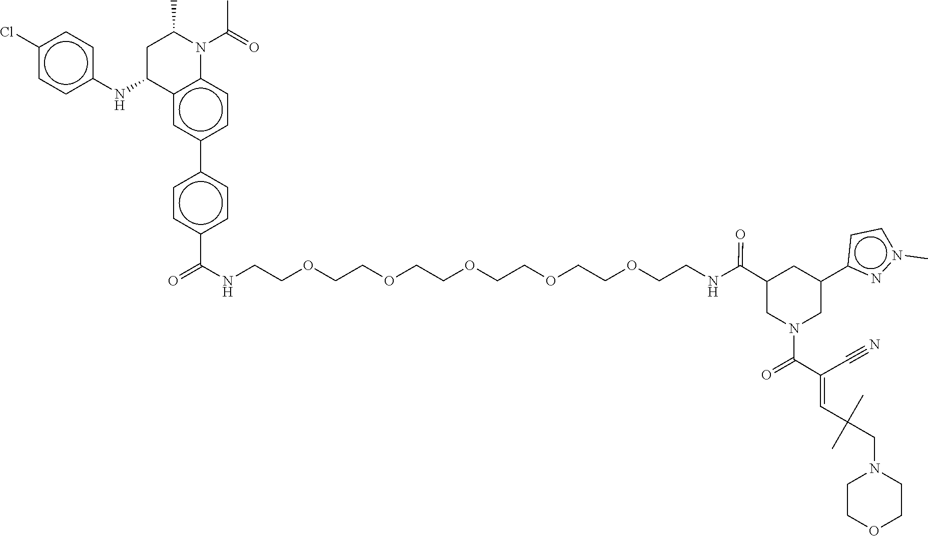 | |
| 13at | 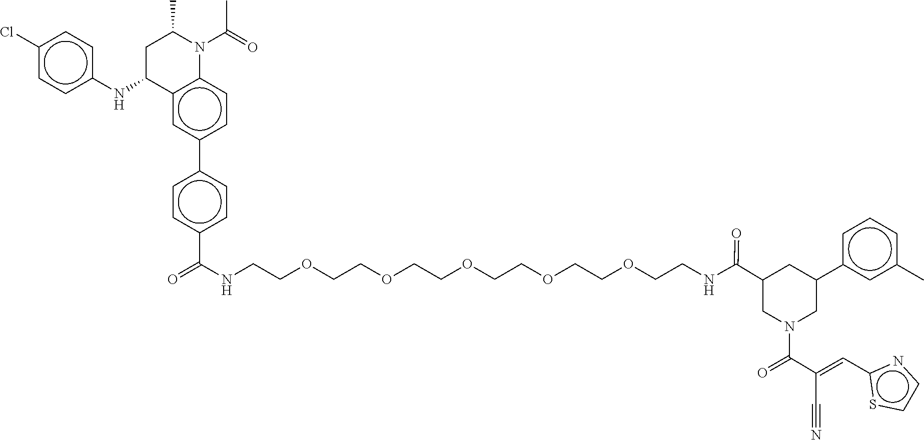 | [M]⁺ 1060.2 and [M + 2]⁺ 1116.2 |
| 13au | 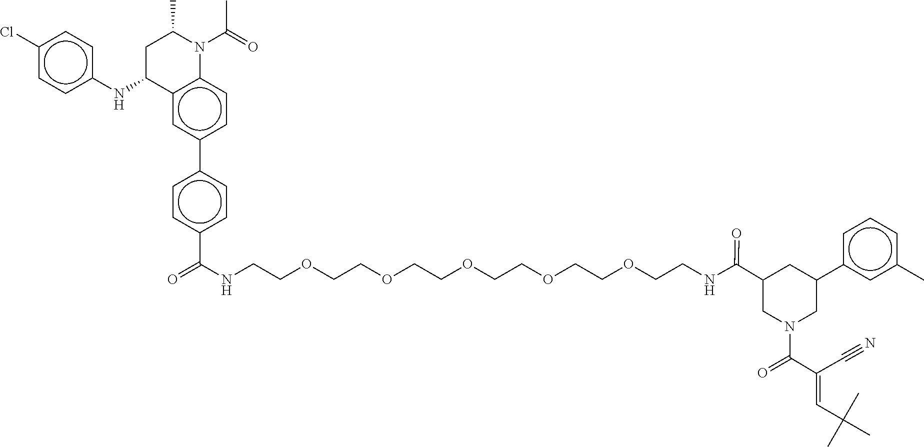 | [M]⁺ 1033.2 and [M + 2]⁺ 1033.2 |
| 13av | 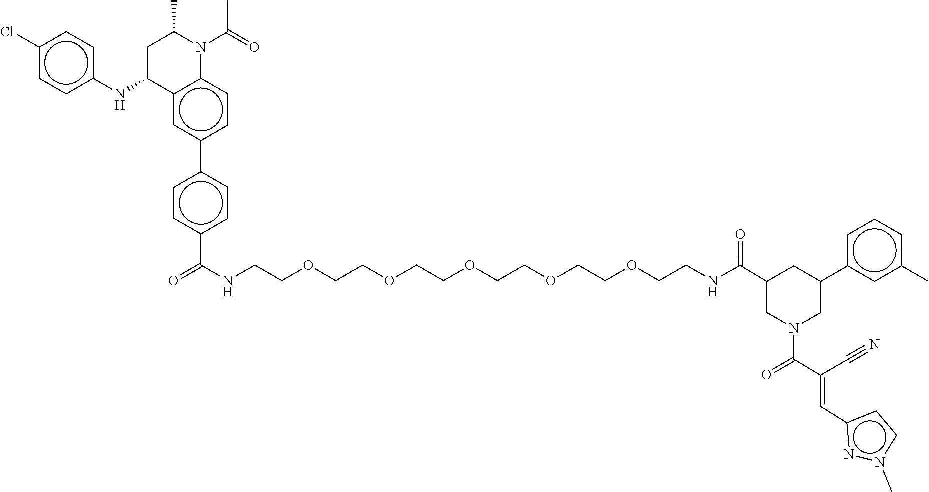 | [M]⁺ 1057.2 and [M + 2]⁺ 1059.2 |
| 13aw | 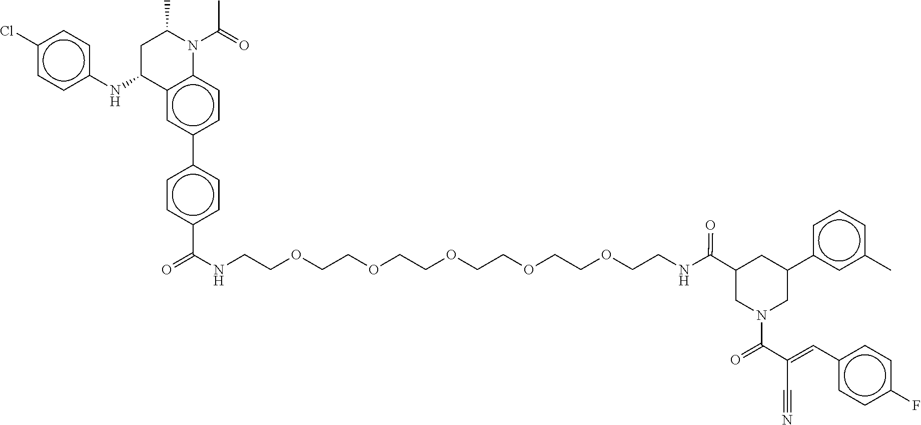 | [M]⁺ 1071.2 and [M + 2]⁺ 1073.2 |
| 13ax | 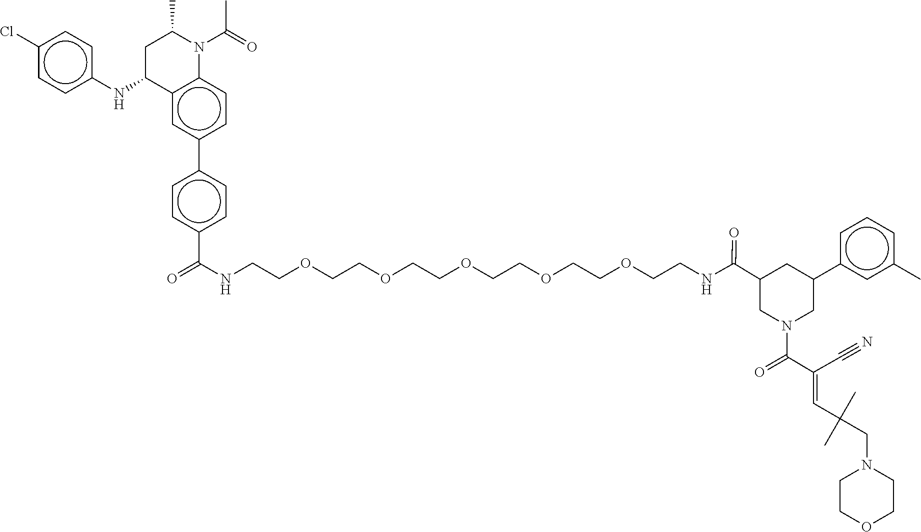 | [M]⁺ 1118.2 and [M + 2]⁺ 1120.2 |

### Piperidine—Pyrrolidine-Based Warheads

Further examples of bifunctional degraders comprising a piperidine-pyrrolidine-based moiety as Z are illustrated below.

Overviews of various exemplary synthetic methods that may be used to provide these compounds are shown below.

### Overview of Synthetic Pathway—Scheme 7

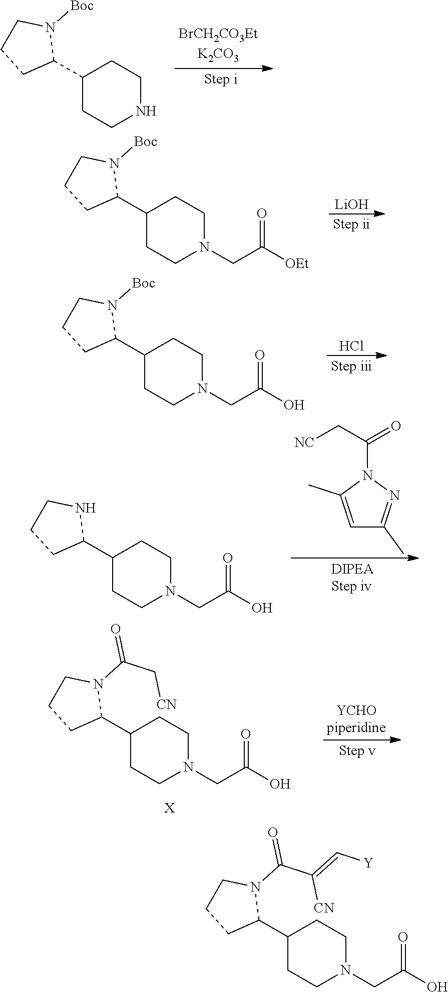

### Overview of Synthetic Pathway—Scheme 8

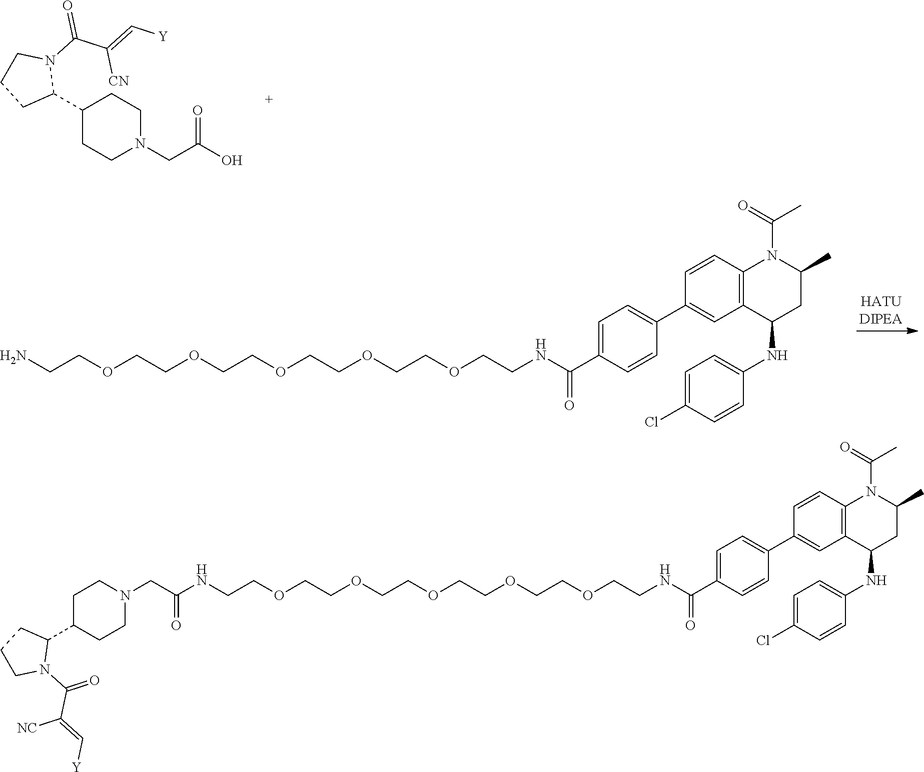

Synthesis from Commercially Available Mono-Boc Diamines (Scheme 7)

Commercially available mono-N-Boc protected diamines are alkylated (ethyl bromoacetate, K₂CO₃ in DMF or MeCN). Hydrolysis of the ester group (LiOH in THF/H₂O) affords the carboxylic acid. Deprotection of the N-Boc amine (HCl in DCM/dioxane) delivers the free amino acid. Acylation using 1 cyanoacetyl-3,5-dimethylpyrazole and DIPEA in dioxane or DMF affords the precursors X for the Knoevenagel reaction, which can be carried on using aldehydes Y shown below in ethanol at r.t. (or THF at 40 to 70° C.) using piperidine as catalyst.

Precursors X and aldehydes Y to be used in the Knoevenagel reaction (step v, Scheme 7) are shown below.

X

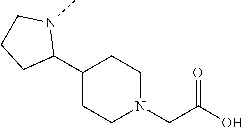

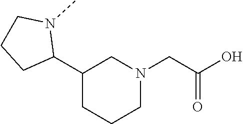

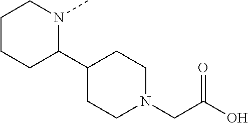

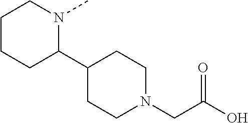

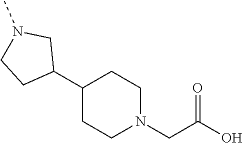

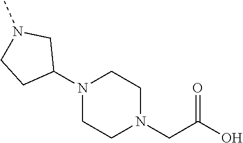

Aldehyde Y

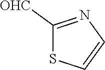

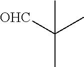

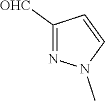

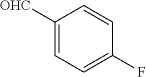

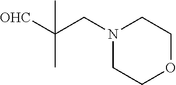

### Examples of Bifunctional Compounds

Synthesis of the final bifunctional degraders is illustrated in scheme 8.

Cyanoacrylamide acids and amine-linker functionalised target protein binding ligand are dissolved in DMF and treated with HATU and DIPEA at room temperature to afford the bifunctional compounds.

An example compound that are made in accordance with the method illustrated in scheme 8 are shown below.

| Ex- | |
|---|---|
| ample | |
| No. | Structure |
| 14a | 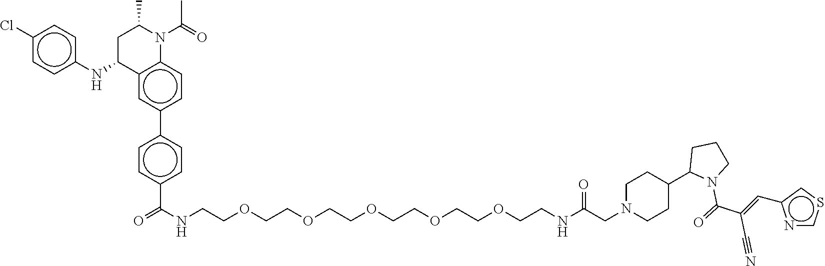 |

### Pyrrolidine-Based Warheads

Further examples of bifunctional degraders comprising a pyrrolidine-based moiety as Z are illustrated below.

Overviews of various exemplary synthetic methods that may be used to provide these compounds are shown below.

### Overview of Synthetic Pathway—Scheme 9

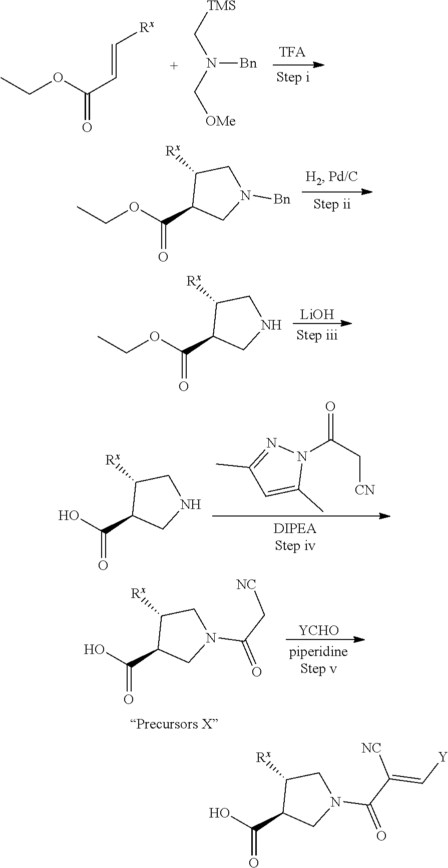

### Overview of Synthetic Pathway—Scheme 10

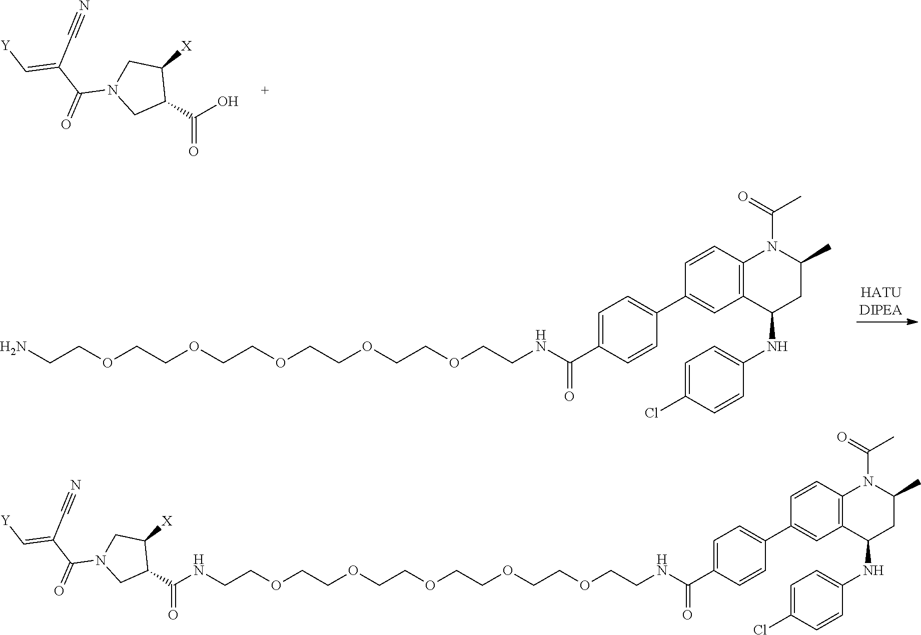

Synthesis from commercially available acrylate esters (scheme 9):

Acrylate esters are treated with N-benzyl-1-methoxy-N-((trimethylsilyl)methyl) methanamine and TFA in toluene to afford the trans-3,4-disubstituted N-benzyl-pyrrolidines. Cleavage of the benzyl group (H₂, Pd(OH)₂/C in ethanol or methanol) affords the free pyrrolidine analogues. Hydrolysis of the ester group (LiOH in THF/H₂O) affords the free amino acids. Acylation using 1 cyanoacetyl-3,5-dimethylpyrazole and DIPEA in dioxane or DMF affords the precursors X for the Knoevenagel reaction, which can be carried on using aldehydes Y shown below in ethanol at r.t. (or THF at 40 to 70° C.) using piperidine as catalyst.

Precursors X and aldehydes Y to be used in the Knoevenagel reaction (step v, Scheme 9) are shown below.

X

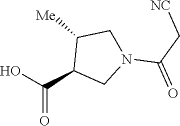 [SMILES: C[C@@H]1CN(C(=O)CC#N)C[C@H]1C(=O)O]

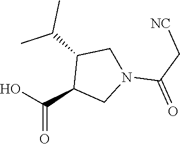 [SMILES: CC(C)[C@@H]1CN(C(=O)CC#N)C[C@H]1C(=O)O]

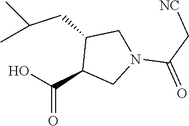 [SMILES: CC(C)C[C@@H]1CN(C(=O)CC#N)C[C@H]1C(=O)O]

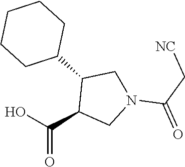 [SMILES: N#CCC(=O)N1C[C@@H](C(=O)O)[C@H](C2CCCCC2)C1]

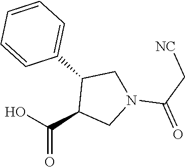 [SMILES: N#CCC(=O)N1C[C@@H](C(=O)O)[C@H](c2ccccc2)C1]

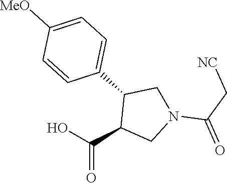 [SMILES: COc1ccc([C@@H]2CN(C(=O)CC#N)C[C@H]2C(=O)O)cc1]

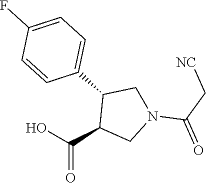 [SMILES: N#CCC(=O)N1C[C@@H](C(=O)O)[C@H](c2ccc(F)cc2)C1]

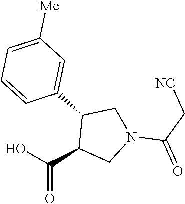 [SMILES: Cc1cccc([C@@H]2CN(C(=O)CC#N)C[C@H]2C(=O)O)c1]

Aldehyde Y

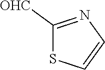

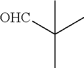

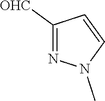

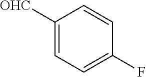

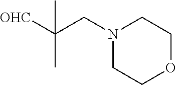

### Example: ethyl 1-benzyl-4-(m-tolyl)pyrrolidine-3-carboxylate

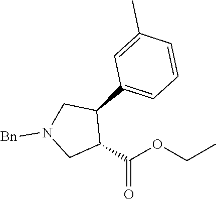 [SMILES: CCOC(=O)[C@@H]1CN(Cc2ccccc2)C[C@H]1c1cccc(C)c1]

To a stirred solution of ethyl (E)-3-(m-tolyl) acrylate (5.00 g, 26.3 mmol) in toluene (50 mL) was added N-benzyl-1-methoxy-N-((trimethylsilyl)methyl) methanamine (6.24 g, 26.3 mmol) at 25° C. The reaction mixture was cooled to 0° C. and then was added TFA (2.63 mL, 2.63 mmol) (1M in DCM) dropwise at 0° C. The reaction mixture was stirred for at 25° C. for 16 h under N₂ atmosphere. The progress of reaction mixture was monitored by TLC. The reaction mixture was diluted with water (50 mL), extracted with ethyl acetate (100 mL×2). Combined organic layer was washed with brine solution (50 mL), dried over Na₂SO₄ and filtered, concentrated under vacuum. The crude residue was purified by flash column chromatography, using 0 to 10% ethyl acetate in hexane to afford pure product ethyl 1-benzyl-4-(m-tolyl)pyrrolidine-3-carboxylate (8 g, 24.49 mmol, 93% yield) as a pale yellow liquid. LCMS: m/z=[M+H]⁺ 324.2; ¹H-NMR (400 MHz, DMSO-d6): δ 7.40-7.32 (m, 4H), 7.29-7.13 (m, 4H), 7.05 (d, J=0.8, 1H), 4.23-4.12 (m, 2H), 3.72-3.65 (m, 2H), 3.09-2.90 (m, 6H), 2.35 (s, 3H), 1.37-1.25 (m, 3H).

| | | m/z |
|---|---|---|
| Example number | Structure/Preparation | (M + H)⁺ |
| 1 | 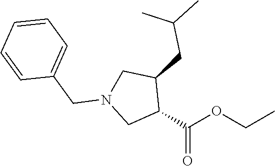 [SMILES: CCOC(=O)[C@@H]1CN(Cc2ccccc2)C[C@H]1CC(C)C] | 291.2 |
| 2 | 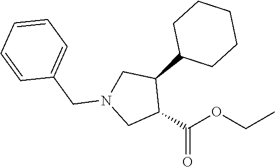 [SMILES: CCOC(=O)[C@@H]1CN(Cc2ccccc2)C[C@H]1C1CCCCC1] | 316.2 |
| 3 | 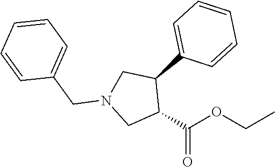 [SMILES: CCOC(=O)[C@@H]1CN(Cc2ccccc2)C[C@H]1c1ccccc1] | 311.2 |
| 4 | 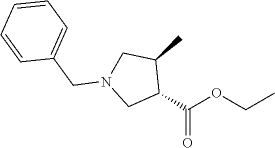 [SMILES: CCOC(=O)[C@@H]1CN(Cc2ccccc2)C[C@H]1C] | 248.1 |

### Ethyl 4-(m-tolyl)pyrrolidine-3-carboxylate

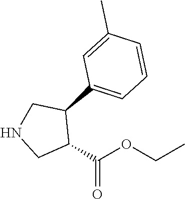 [SMILES: CCOC(=O)[C@@H]1CNC[C@H]1c1cccc(C)c1]

To a stirred solution of ethyl 1-benzyl-4-(m-tolyl) pyrrolidine-3-carboxylate (5.00 g, 15.5 mmol) in EtOH (40 mL) was added palladium hydroxide on carbon (1.085 g, 7.73 mmol) at 25° C. and the reaction was stirred for at 25° C. for 16 h under H₂ atmosphere. The progress of reaction mixture was monitored by TLC. The reaction mixture was filtered through celite pad and washed with ethanol, the filtrate was concentrated under vacuum to afford the product ethyl 4-(m-tolyl) pyrrolidine-3-carboxylate (3.5 g, 14.70 mmol, 95% yield) as a pale yellow liquid. LCMS: m/z=[M+H]⁺ 234.2; ¹H-NMR (400 MHz, DMSO-d6): δ 7.17 (t, J=8, 1H), 7.08-7.00 (m, 3H), 4.10-4.00 (m, 2H), 3.32-3.22 (m, 2H), 3.13-2.91 (m, 3H), 2.70-2.69 (m, 1H), 2.28 (s, 3H), 1.25-1.17 (m, 3H).

### Additional Example

| | | m/z |
|---|---|---|
| Example number | Structure/Preparation | (M + H)⁺ |
| 1 | 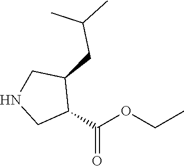 | 200.2 |
| 2 | 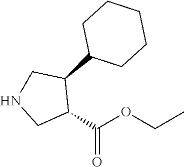 [SMILES: CCOC(=O)[C@@H]1CNC[C@H]1C1CCCCC1] | 226.2 |
| 3 | 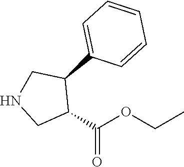 [SMILES: CCOC(=O)[C@@H]1CNC[C@H]1c1ccccc1] | 220.1 |
| 4 | 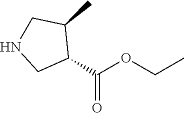 | 158.1 |

Any compounds used but not described in the experimental are commercially available.

Step-4: Synthesis of ethyl 1-(2-cyanoacetyl)-4-(m-tolyl)pyrrolidine-3-carboxylate:

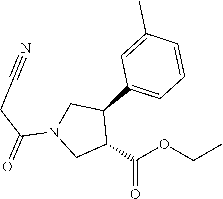 [SMILES: CCOC(=O)[C@@H]1CN(C(=O)CC#N)C[C@H]1c1cccc(C)c1]

To a stirred solution of ethyl 4-(m-tolyl) pyrrolidine-3-carboxylate (100 mg, 0.429 mmol) in Acetonitrile (2 mL) were added DIPEA (0.150 mL, 0.857 mmol), 3-(3,5-dimethyl-1H-pyrazol-1-yl)-3-oxopropanenitrile (84 mg, 0.514 mmol) at 25° C. The reaction mixture was stirred at 60° C. for 3 h under N₂ atmosphere. The progress of reaction mixture was monitored by TLC. The reaction mixture was diluted with water (2 mL), extracted with ethyl acetate (10 mL×2). Combined organic layer was dried over Na₂SO₄ and filtered, and concentrated under vacuum. The crude residue was purified by flash column using 0 to 20% ethyl acetate in hexanes to afford the product ethyl 1-(2-cyanoacetyl)-4-(m-tolyl)pyrrolidine-3-carboxylate (90 mg, 0.191 mmol, 44.7% yield) as a yellow semi-solid. LCMS: m/z=[M+H]⁺ 301.2; ¹H-NMR (400 MHz, DMSO-d6): δ 7.25-7.06 (m, 4H), 4.10-3.90 (m, 7H), 3.68-3.48 (m, 3H), 2.29 (s, 3H), 1.25-1.17 (m, 3H).

| | | m/z |
|---|---|---|
| Example number | Structure/Preparation | (M + H)⁺ |
| 1 | 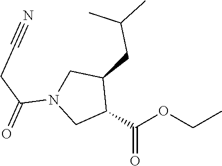 [SMILES: CCOC(=O)[C@@H]1CN(C(=O)CC#N)C[C@H]1CC(C)C] | 267.1 |
| 2 | 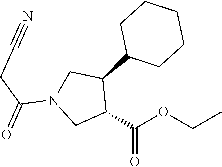 [SMILES: CCOC(=O)[C@@H]1CN(C(=O)CC#N)C[C@H]1C1CCCCC1] | 293.1 |
| 3 | 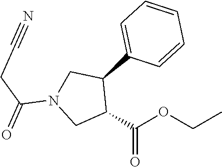 [SMILES: CCOC(=O)[C@@H]1CN(C(=O)CC#N)C[C@H]1c1ccccc1] | 287.1 |
| 4 | 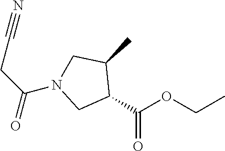 [SMILES: CCOC(=O)[C@@H]1CN(C(=O)CC#N)C[C@H]1C] | 225.1 |
| 5 | 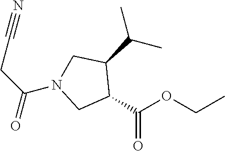 [SMILES: CCOC(=O)[C@@H]1CN(C(=O)CC#N)C[C@H]1C(C)C] | 253.2 |
| 6 | 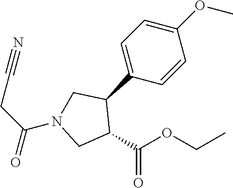 [SMILES: CCOC(=O)[C@@H]1CN(C(=O)CC#N)C[C@H]1c1ccc(OC)cc1] | 317.2 |
| 7 | 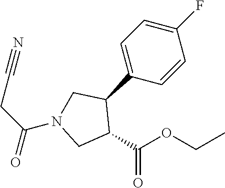 [SMILES: CCOC(=O)[C@@H]1CN(C(=O)CC#N)C[C@H]1c1ccc(F)cc1] | 305.2 |

### 1-(2-cyanoacetyl)-4-(m-tolyl) pyrrolidine-3-carboxylic acid (Pyracid 0)

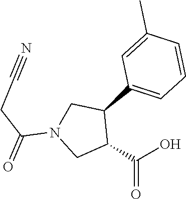 [SMILES: Cc1cccc([C@@H]2CN(C(=O)CC#N)C[C@H]2C(=O)O)c1]

To a stirred solution of ethyl (1-(2-cyanoacetyl)-4-(m-tolyl)pyrrolidine-3-carboxylate (2.3 g, 7.66 mmol) in MeOH (15 ml), Water (15.00 ml) was added LiOH (0.275 g, 11.49 mmol) at 0° C. The reaction mixture was stirred at 25° C. for 16 h under N₂ atmosphere. The progress of reaction mixture was monitored by TLC. The reaction mixture was concentrated under vacuum and diluted with water, acidified with 1.5N HCl solution to pH=2. The resulting precipitate was filtered, washed with water and dried under vacuum to afford pure product (1-(2-cyanoacetyl)-4-(m-tolyl)pyrrolidine-3-carboxylic acid (1 g, 3.67 mmol, 48.0% yield) as brown solid. LCMS: m/z=[M+H]⁺ 373.0; 1H-NMR (400 MHz, DMSO-d6): δ 12.5 (s, 1H), 7.24-7.18 (m, 1H), 7.17-7.06 (m, 3H), 4.00-3.85 (m, 4H), 3.60-3.18 (m, 4H), 2.29 (s, 3H).

| Example | | m/z |
|---|---|---|
| number | Structure/Preparation | (M + H)⁺ |
| Pyracid 1 | 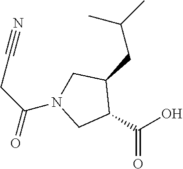 [SMILES: CC(C)C[C@@H]1CN(C(=O)CC#N)C[C@H]1C(=O)O] | 239.1 |
| Pyracid 2 | 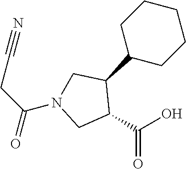 [SMILES: N#CCC(=O)N1C[C@@H](C(=O)O)[C@H](C2CCCCC2)C1] | 265.1 |
| Pyracid 3 | 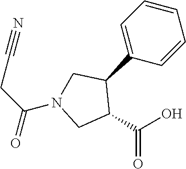 [SMILES: N#CCC(=O)N1C[C@@H](C(=O)O)[C@H](c2ccccc2)C1] | 259.1 |
| Pyracid 4 | 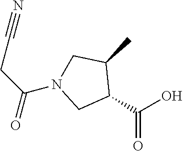 | 197.1 |
| Pyracid 5 | 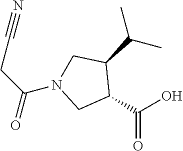 [SMILES: CC(C)[C@@H]1CN(C(=O)CC#N)C[C@H]1C(=O)O] | 225.2 |
| Pyracid 6 | 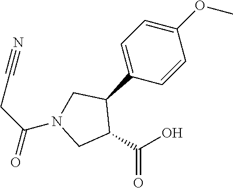 [SMILES: COc1ccc([C@@H]2CN(C(=O)CC#N)C[C@H]2C(=O)O)cc1] | 288.2 |
| Pyracid 7 | 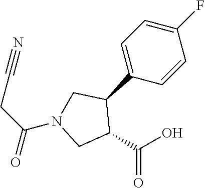 [SMILES: N#CCC(=O)N1C[C@@H](C(=O)O)[C@H](c2ccc(F)cc2)C1] | 277.1 |

### 1-((E)-2-cyano-4,4-dimethylpent-2-enoyl)-4-(m-tolyl)pyrrolidine-3-carboxylic acid (Pyracid 8)

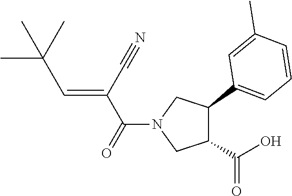 [SMILES: Cc1cccc([C@@H]2CN(C(=O)/C(C#N)=C/C(C)(C)C)C[C@H]2C(=O)O)c1]

To a stirred solution of 1-(2-cyanoacetyl)-4-(m-tolyl) pyrrolidine-3-carboxylic acid (100 mg, 0.367 mmol) in EtOH (2 mL) were added piperidine (62.5 mg, 0.734 mmol), pivalaldehyde (95 mg, 1.102 mmol) at 25° C. the reaction mixture was stirred at 25° C. for 16 h under N₂ atmosphere. The progress of reaction mixture was monitored by TLC. The reaction mixture was concentrated to afford crude. The crude obtained was purified by flash column using 0 to 10% MeOH in DCM to afford product 1-((Z)-2-cyano-4,4-dimethylpent-2-enoyl)-4-(m-tolyl)pyrrolidine-3-carboxylic acid (27 mg, 0.021 mmol, 5.62% yield) as a brown semi solid. LCMS: m/z=[M+H]⁺ 341.1; 1H-NMR (400 MHz, DMSO-d6): δ 12.62 (s, 1H), 7.25-7.04 (m, 5H), 3.99-3.51 (m, 5H), 3.43-3.40 (m, 1H), 2.29 (s, 3H), 1.25 (d, J=16, 9H).

| | | m/z |
|---|---|---|
| Example number | Structure/Preparation | (M + H)⁺ |
| Pyracid 9 | 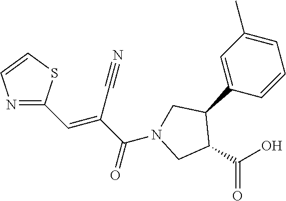 [SMILES: Cc1cccc([C@@H]2CN(C(=O)/C(C#N)=C/c3nccs3)C[C@H]2C(=O)O)c1] | 368.1 |
| Pyracid 10 | 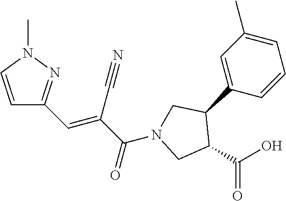 [SMILES: Cc1cccc([C@@H]2CN(C(=O)/C(C#N)=C/c3ccn(C)n3)C[C@H]2C(=O)O)c1] | 365.1 |
| Pyracid 11 | 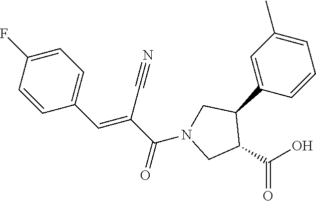 [SMILES: Cc1cccc([C@@H]2CN(C(=O)/C(C#N)=C/c3ccc(F)cc3)C[C@H]2C(=O)O)c1] | 379.1 |
| Pyracid 12 | 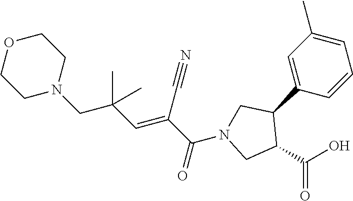 [SMILES: Cc1cccc([C@@H]2CN(C(=O)/C(C#N)=C/C(C)(C)CN3CCOCC3)C[C@H]2C(=O)O)c1] | 426.1 |
| Pyracid 13 | 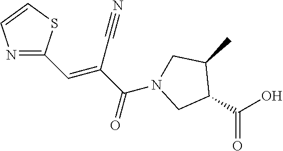 [SMILES: C[C@@H]1CN(C(=O)/C(C#N)=C/c2nccs2)C[C@H]1C(=O)O] | 292.1 |
| Pyracid 14 | 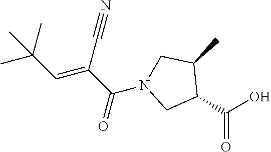 [SMILES: C[C@@H]1CN(C(=O)/C(C#N)=C/C(C)(C)C)C[C@H]1C(=O)O] | 265.1 |
| Pyracid 15 | 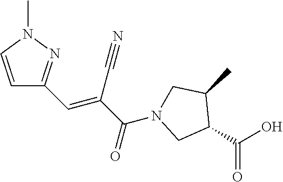 [SMILES: C[C@@H]1CN(C(=O)/C(C#N)=C/c2ccn(C)n2)C[C@H]1C(=O)O] | 288.1 |
| Pyracid 16 | 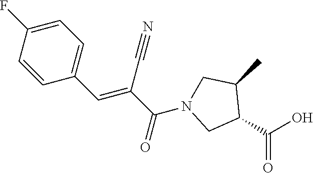 [SMILES: C[C@@H]1CN(C(=O)/C(C#N)=C/c2ccc(F)cc2)C[C@H]1C(=O)O] | 303.1 |
| Pyracid 17 | 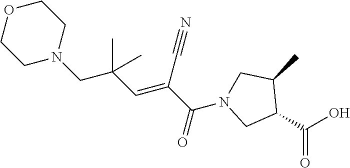 [SMILES: C[C@@H]1CN(C(=O)/C(C#N)=C/C(C)(C)CN2CCOCC2)C[C@H]1C(=O)O] | 350.1 |
| Pyracid 18 | 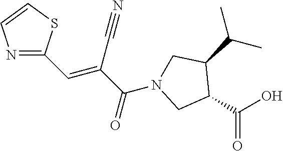 [SMILES: CC(C)[C@@H]1CN(C(=O)/C(C#N)=C/c2nccs2)C[C@H]1C(=O)O] | 320.1 |
| Pyracid 19 | 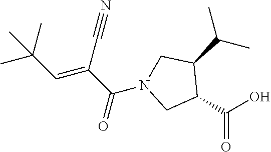 [SMILES: CC(C)[C@@H]1CN(C(=O)/C(C#N)=C/C(C)(C)C)C[C@H]1C(=O)O] | 293.1 |
| Pyracid 20 | 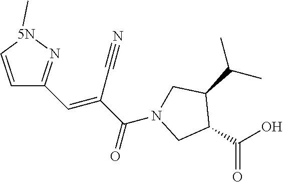 | 317.1 |
| Pyracid 21 | 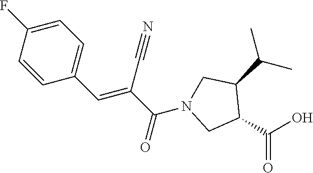 [SMILES: CC(C)[C@@H]1CN(C(=O)/C(C#N)=C/c2ccc(F)cc2)C[C@H]1C(=O)O] | 331.1 |
| Pyracid 22 | 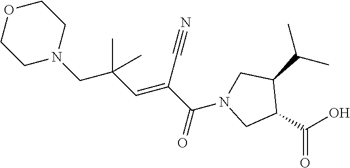 [SMILES: CC(C)[C@@H]1CN(C(=O)/C(C#N)=C/C(C)(C)CN2CCOCC2)C[C@H]1C(=O)O] | 378.1 |
| Pyracid 23 | 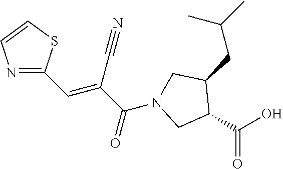 [SMILES: CC(C)C[C@@H]1CN(C(=O)/C(C#N)=C/c2nccs2)C[C@H]1C(=O)O] | 334.1 |
| Pyracid 24 | 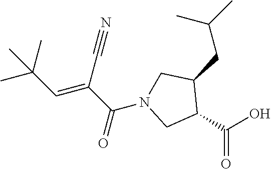 [SMILES: CC(C)C[C@@H]1CN(C(=O)/C(C#N)=C/C(C)(C)C)C[C@H]1C(=O)O] | 3073.1 |
| Pyracid 25 | 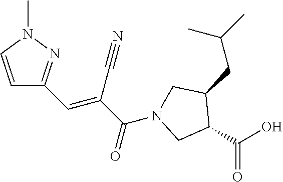 [SMILES: CC(C)C[C@@H]1CN(C(=O)/C(C#N)=C/c2ccn(C)n2)C[C@H]1C(=O)O] | 331.1 |
| Pyracid 26 | 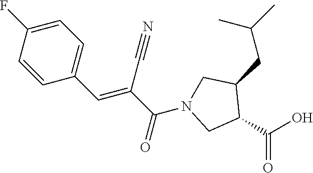 [SMILES: CC(C)C[C@@H]1CN(C(=O)/C(C#N)=C/c2ccc(F)cc2)C[C@H]1C(=O)O] | 345.1 |
| Pyracid 27 | 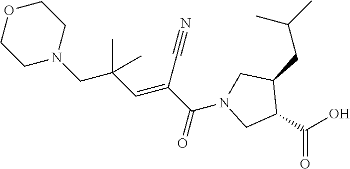 [SMILES: CC(C)C[C@@H]1CN(C(=O)/C(C#N)=C/C(C)(C)CN2CCOCC2)C[C@H]1C(=O)O] | 392.1 |
| Pyracid 28 | 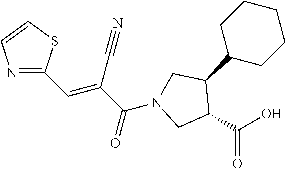 [SMILES: N#C/C(=C\c1nccs1)C(=O)N1C[C@@H](C(=O)O)[C@H](C2CCCCC2)C1] | 360.1 |
| Pyracid 29 | 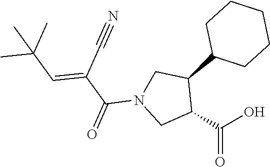 [SMILES: CC(C)(C)/C=C(\C#N)C(=O)N1C[C@@H](C(=O)O)[C@H](C2CCCCC2)C1] | 332.1 |
| Pyracid 30 | 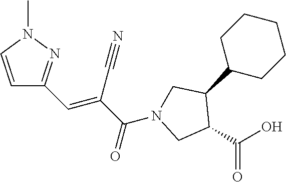 [SMILES: Cn1ccc(/C=C(\C#N)C(=O)N2C[C@@H](C(=O)O)[C@H](C3CCCCC3)C2)n1] | 357.1 |
| Pyracid 31 | 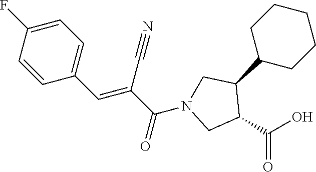 [SMILES: N#C/C(=C\c1ccc(F)cc1)C(=O)N1C[C@@H](C(=O)O)[C@H](C2CCCCC2)C1] | 371.1 |
| Pyracid 32 | 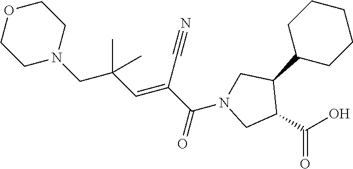 [SMILES: CC(C)(/C=C(\C#N)C(=O)N1C[C@@H](C(=O)O)[C@H](C2CCCCC2)C1)CN1CCOCC1] | 418.1 |
| Pyracid 33 | 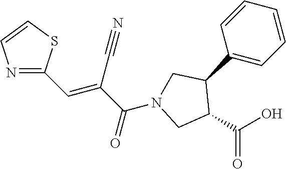 [SMILES: N#C/C(=C\c1nccs1)C(=O)N1C[C@@H](C(=O)O)[C@H](c2ccccc2)C1] | 354.1 |
| Pyracid 34 | 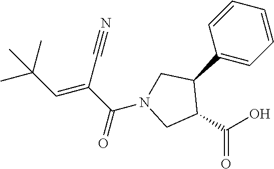 [SMILES: CC(C)(C)/C=C(\C#N)C(=O)N1C[C@@H](C(=O)O)[C@H](c2ccccc2)C1] | 327.1 |
| Pyracid 35 | 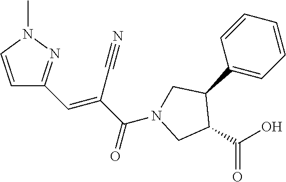 [SMILES: Cn1ccc(/C=C(\C#N)C(=O)N2C[C@@H](C(=O)O)[C@H](c3ccccc3)C2)n1] | 351.1 |
| Pyracid 36 | 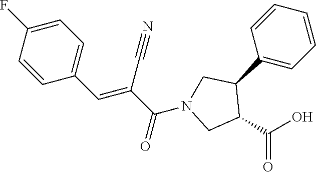 [SMILES: N#C/C(=C\c1ccc(F)cc1)C(=O)N1C[C@@H](C(=O)O)[C@H](c2ccccc2)C1] | 365.1 |
| Pyracid 37 | 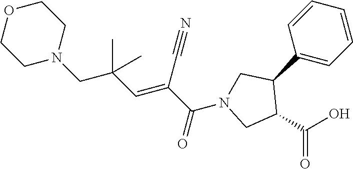 [SMILES: CC(C)(/C=C(\C#N)C(=O)N1C[C@@H](C(=O)O)[C@H](c2ccccc2)C1)CN1CCOCC1] | 412.1 |
| Pyracid 38 | 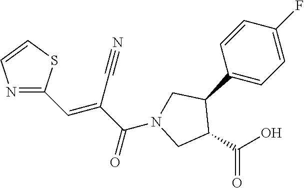 [SMILES: N#C/C(=C\c1nccs1)C(=O)N1C[C@@H](C(=O)O)[C@H](c2ccc(F)cc2)C1] | 372.1 |
| Pyracid 39 | 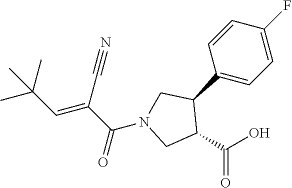 [SMILES: CC(C)(C)/C=C(\C#N)C(=O)N1C[C@@H](C(=O)O)[C@H](c2ccc(F)cc2)C1] | 345.1 |
| Pyracid 40 | 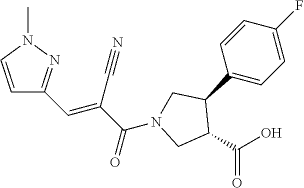 [SMILES: Cn1ccc(/C=C(\C#N)C(=O)N2C[C@@H](C(=O)O)[C@H](c3ccc(F)cc3)C2)n1] | 369.1 |
| Pyracid 41 | 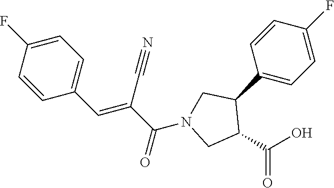 [SMILES: N#C/C(=C\c1ccc(F)cc1)C(=O)N1C[C@@H](C(=O)O)[C@H](c2ccc(F)cc2)C1] | 383.1 |
| Pyracid 42 | 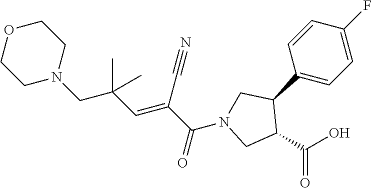 [SMILES: CC(C)(/C=C(\C#N)C(=O)N1C[C@@H](C(=O)O)[C@H](c2ccc(F)cc2)C1)CN1CCOCC1] | 430.1 |
| Pyracid 43 | 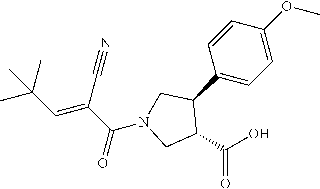 [SMILES: COc1ccc([C@@H]2CN(C(=O)/C(C#N)=C/C(C)(C)C)C[C@H]2C(=O)O)cc1] | 384.1 |
| Pyracid 44 | 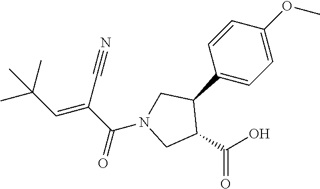 [SMILES: COc1ccc([C@@H]2CN(C(=O)/C(C#N)=C/C(C)(C)C)C[C@H]2C(=O)O)cc1] | 357.1 |
| Pyracid 45 | 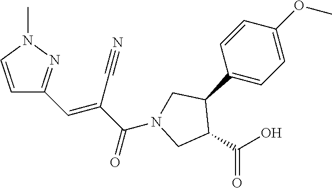 [SMILES: COc1ccc([C@@H]2CN(C(=O)/C(C#N)=C/c3ccn(C)n3)C[C@H]2C(=O)O)cc1] | 381.1 |
| Pyracid 46 | 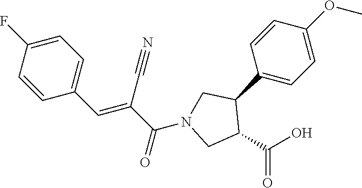 [SMILES: COc1ccc([C@@H]2CN(C(=O)/C(C#N)=C/c3ccc(F)cc3)C[C@H]2C(=O)O)cc1] | 395.1 |
| Pyracid 47 | 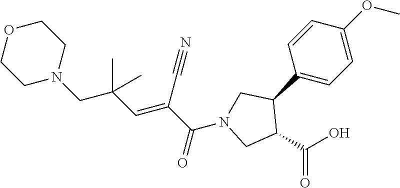 [SMILES: COc1ccc([C@@H]2CN(C(=O)/C(C#N)=C/C(C)(C)CN3CCOCC3)C[C@H]2C(=O)O)cc1] | 442.1 |

### Examples of Bifunctional Compounds:

Synthesis of the final bifunctional degraders is illustrated in scheme 10.

Cyanoacrylamide acids and amine-linker functionalised target protein binding ligand are dissolved in DMF and treated with HATU and DIPEA at room temperature to afford the bifunctional compounds.

Example compounds that are made in accordance with the method illustrated in scheme 10 are shown below.

### N-(1-(4-((2S,4R)-1-acetyl-4-((4-chlorophenyl)amino)-2-methyl-1,2,3,4-tetrahydroquinolin-6-yl)phenyl)-1-oxo-5,8,11,14,17-pentaoxa-2-azanonadecan-19-yl)-1-((E)-2-cyano-4,4-dimethylpent-2-enoyl)-4-(m-tolyl)pyrrolidine-3-carboxamide (15ak)

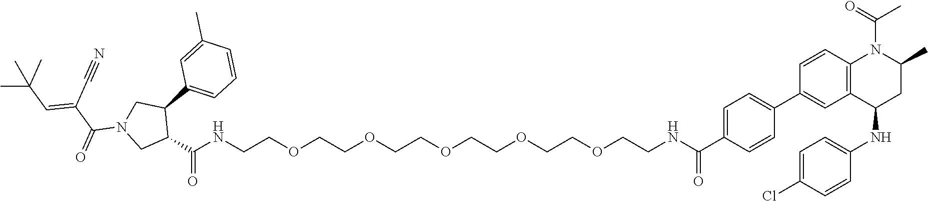 [SMILES: CC(=O)N1c2ccc(-c3ccc(C(=O)NCCOCCOCCOCCOCCOCCNC(=O)[C@@H]4CN(C(=O)/C(C#N)=C/C(C)(C)C)C[C@H]4c4cccc(C)c4)cc3)cc2[C@H](Nc2ccc(Cl)cc2)C[C@@H]1C]

To a stirred solution of 1-((Z)-2-cyano-4,4-dimethylpent-2-enoyl)-4-(m-tolyl)pyrrolidine-3-carboxylic acid (10.99 mg, 0.032 mmol) in DMF (0.5 mL) were added DIPEA (0.015 mL, 0.086 mmol), BOP (14.27 mg, 0.032 mmol) at 25° C. the reaction mixture was stirred for 5 minutes. Then 4-((2S,4R)-1-acetyl-4-((4-chlorophenyl)amino)-2-methyl-1,2,3,4-tetrahydroquinolin-6-yl)-N-(17-amino-3,6,9,12,15-pentaoxaheptadecyl)benzamide (15 mg, 0.022 mmol) was added at 25° C. The reaction mixture was stirred at 25° C. for 16 h under N₂ atmosphere. The progress of reaction mixture was monitored by LCMS. The reaction mixture was diluted with water (10 mL), extracted with ethyl acetate (20 mL×2) washed with brine solution (10 mL), the organic layer was dried over Na₂SO₄ and filtered, concentrated under vacuum to afford the crude product. Crude product was purified by mass directed prep purification (Mobile phase A: 0.1% HCOOH in H₂O, Mobile phase B: 0.1% HCOOH in ACN, Column: X-select C18 250*19.0, 5u. After purification desired fraction were lyophilized to give pure product N-(1-(4-((2S,4R)-1-acetyl-4-((4-chlorophenyl)amino)-2-methyl-1,2,3,4-tetrahydroquinolin-6-yl)phenyl)-1-oxo-5,8,11,14,17-pentaoxa-2-azanonadecan-19-yl)-1-2-cyano-4,4-dimethylpent-2-enoyl)-4-(m-tolyl)pyrrolidine-3-carboxamide (2.6 mg, 2.473 μmol) as a white solid. LCMS: m/z=[M+H]⁺ 1019.2 and [M+2]⁺ 1021.2 (Chloro pattern)

### Additional Examples

| Example | | m/z |
|---|---|---|
| number | Structure | (M + H)⁺ |
| 15a | 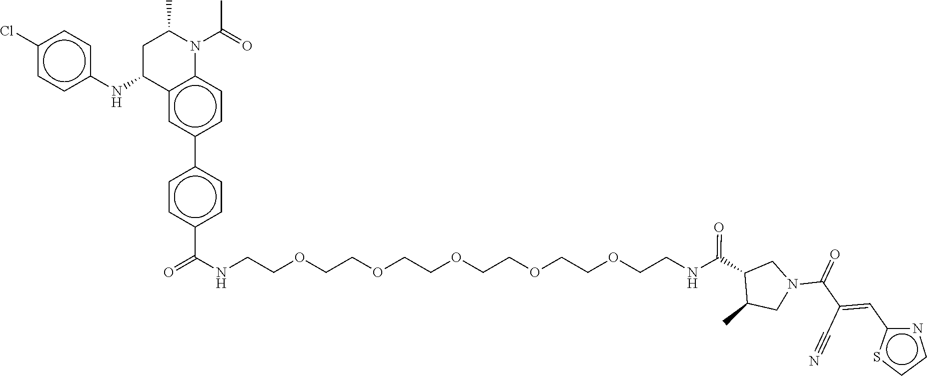 | [M]⁺ 970.2 and [M + 2]⁺ 972.2 |
| 15b | 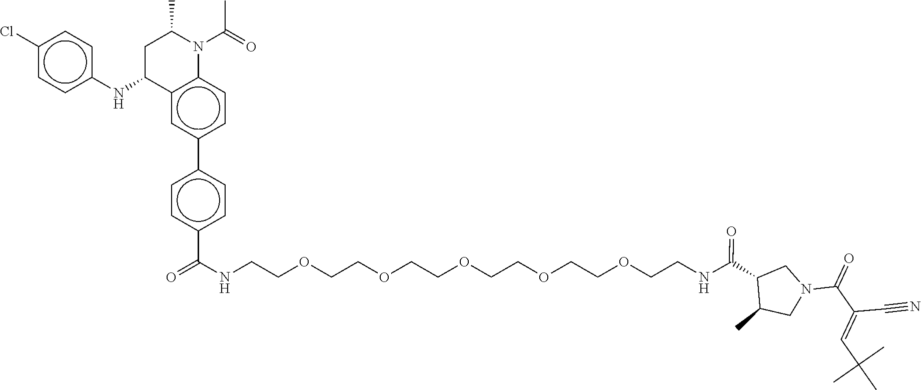 | [M]⁺ 943.2 and [M + 2]⁺ 945.2 |
| 15c | 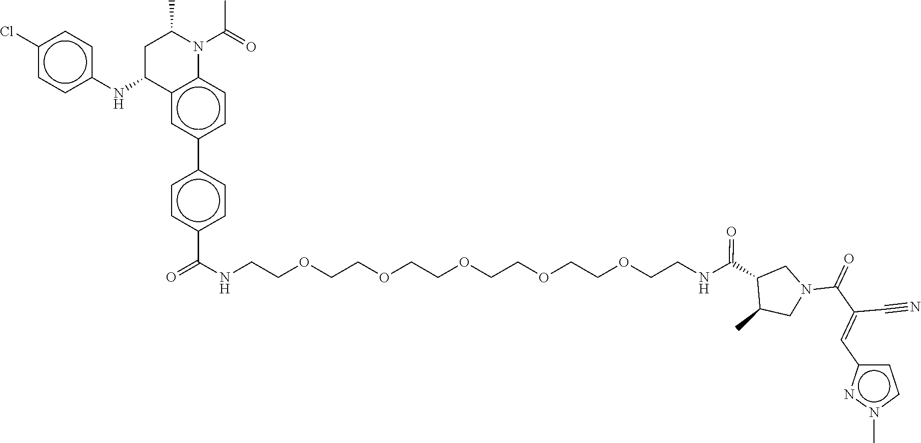 | [M]⁺ 967.2 and [M + 2]⁺ 969.2 |
| 15d | 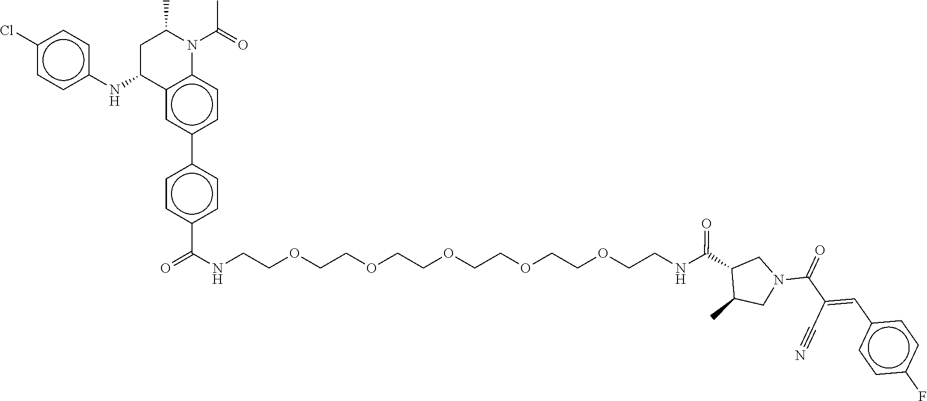 | [M]⁺ 981.1 and [M + 2]⁺ 983.1 |
| 15e | 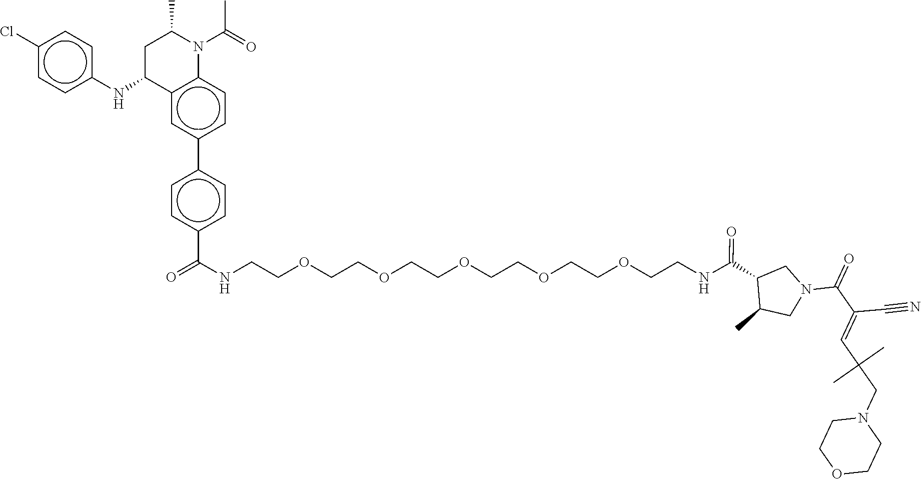 | |
| 15f | 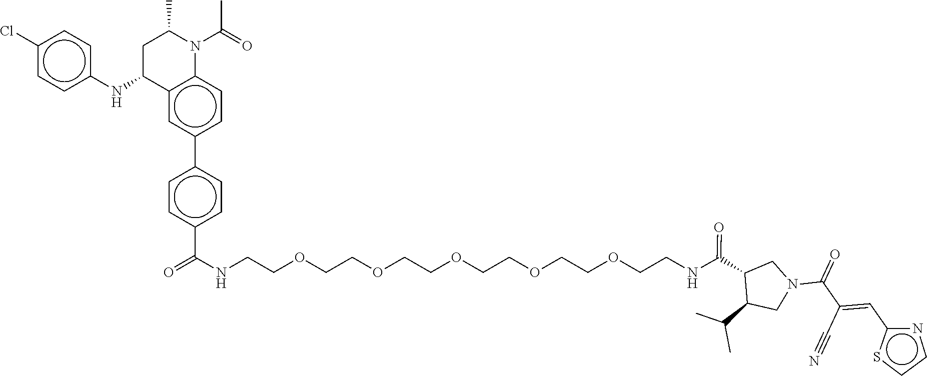 | [M]⁺ 998.2 and [M + 2]⁺ 1000.2 |
| 15g | 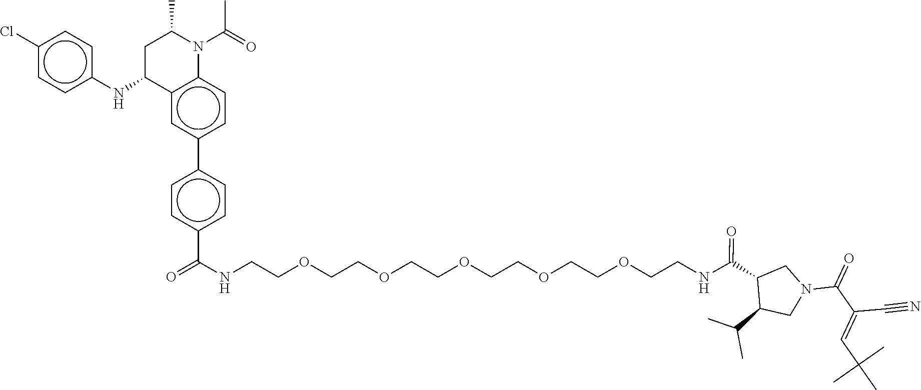 | [M]⁺ 971.2 and [M + 2]⁺ 973.2 |
| 15h | 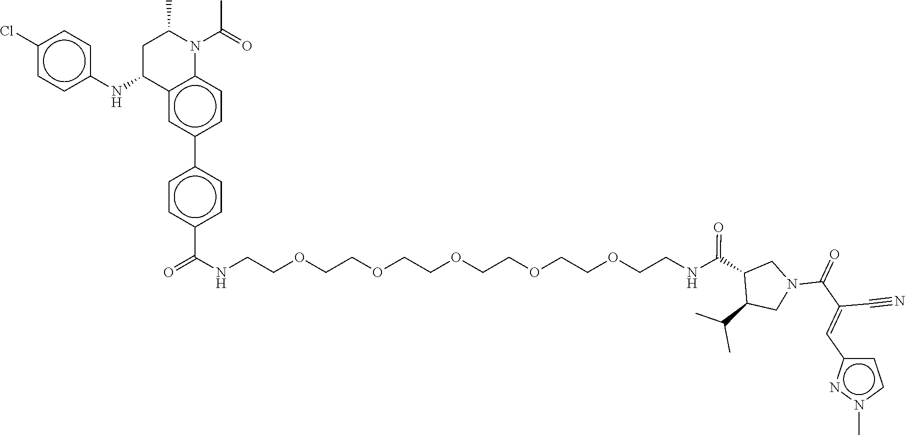 | [M]⁺ 995.2 and [M + 2]⁺ 997.2 |
| 15i | 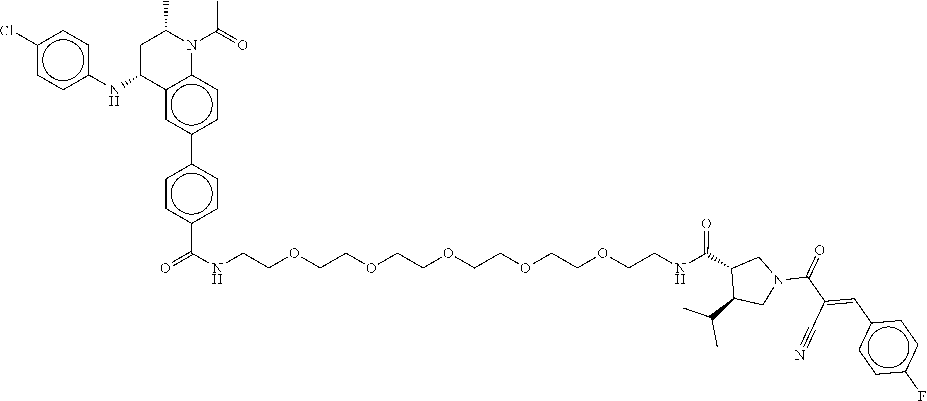 | [M]⁺ 1009.1 and [M + 2]⁺ 1011.1 |
| 15j | 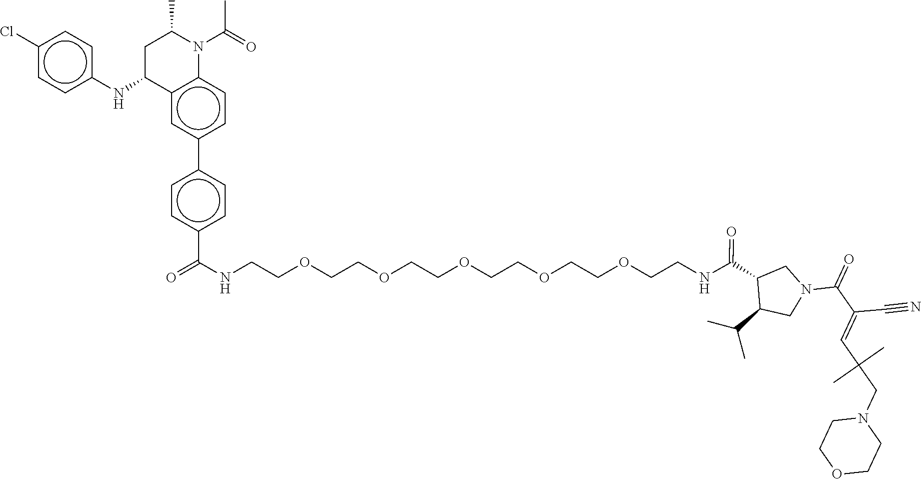 | [M]⁺ 1056.1 and [M + 2]⁺ 1058.1 |
| 15k | 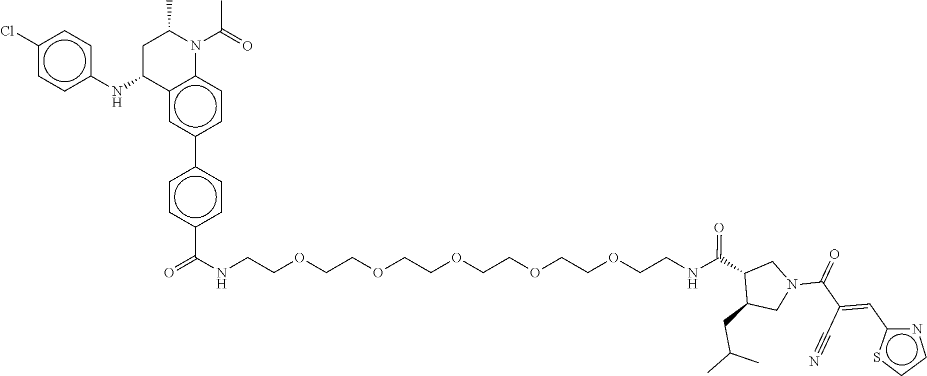 | [M]⁺ 1012.2 and [M + 2]⁺ 1014.2 |
| 15l | 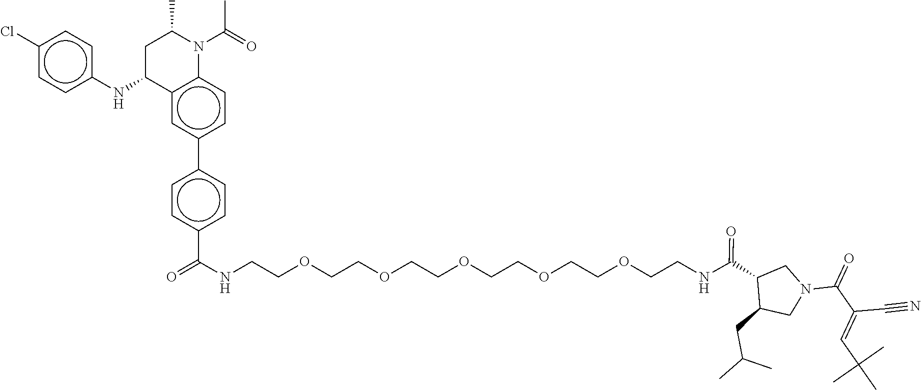 | [M]⁺ 985.2 and [M + 2]⁺ 987.2 |
| 15m | 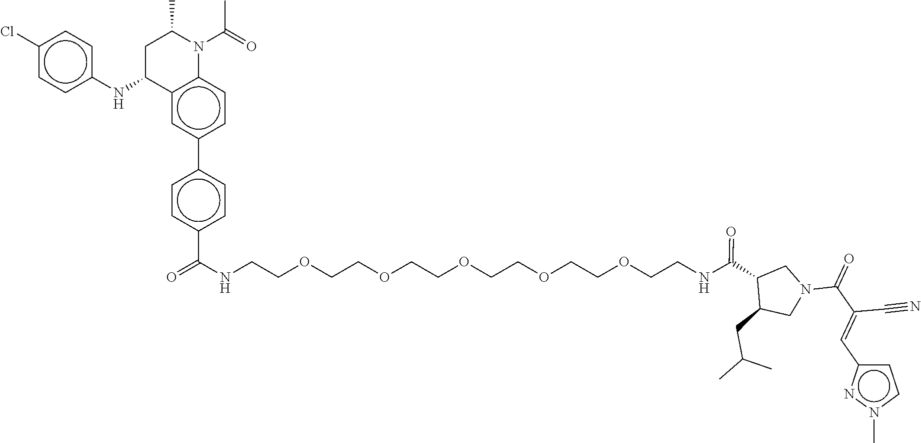 | [M]⁺ 1009.2 and [M + 2]⁺ 1011.2 |
| 15n | 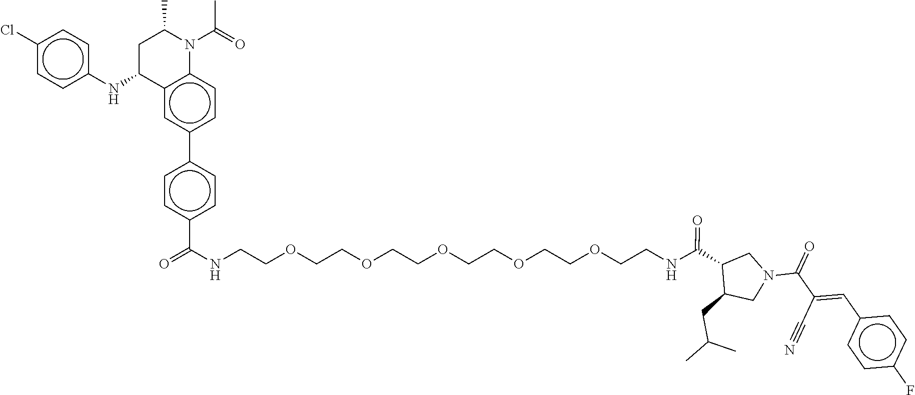 | [M]⁺ 1023.1 and [M + 2]⁺ 1025.1 |
| 15o | 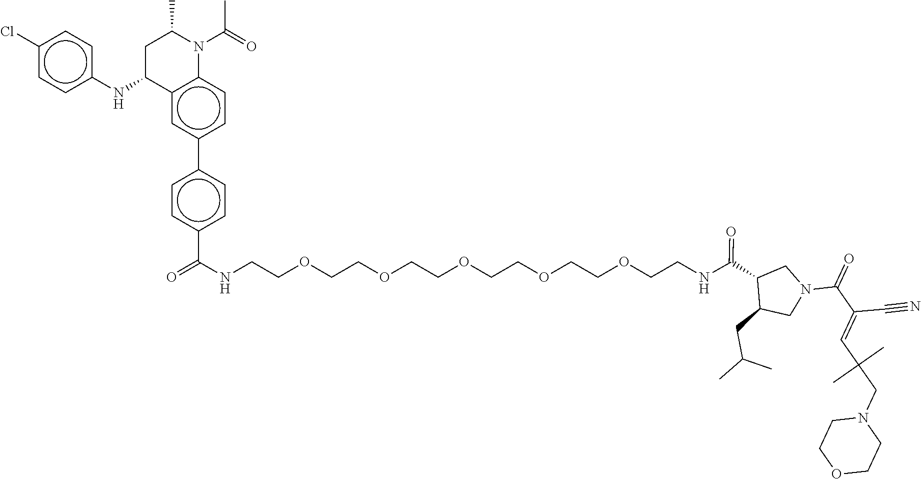 | [M]⁺ 1070.1 and [M + 2]⁺ 1072.1 |
| 15p | 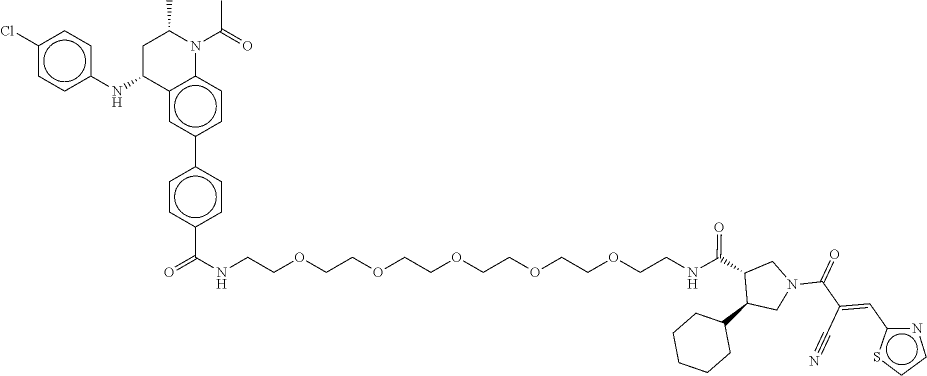 | [M]⁺ 1038.2 and [M + 2]⁺ 1040.2 |
| 15q | 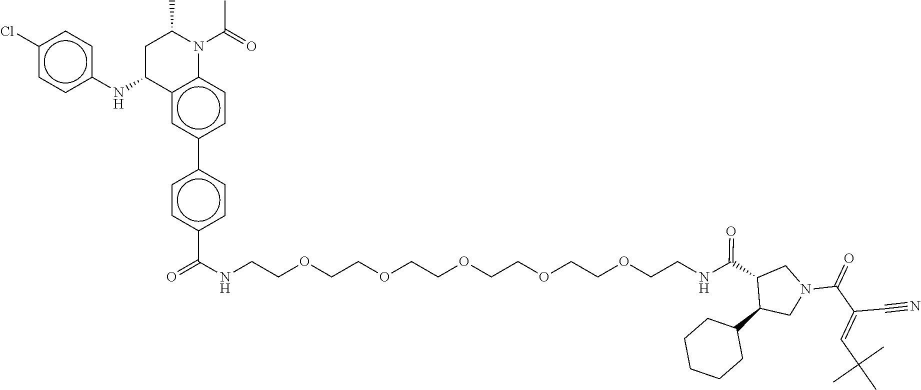 | [M]⁺ 1011.2 and [M + 2]⁺ 1013.2 |
| 15r | 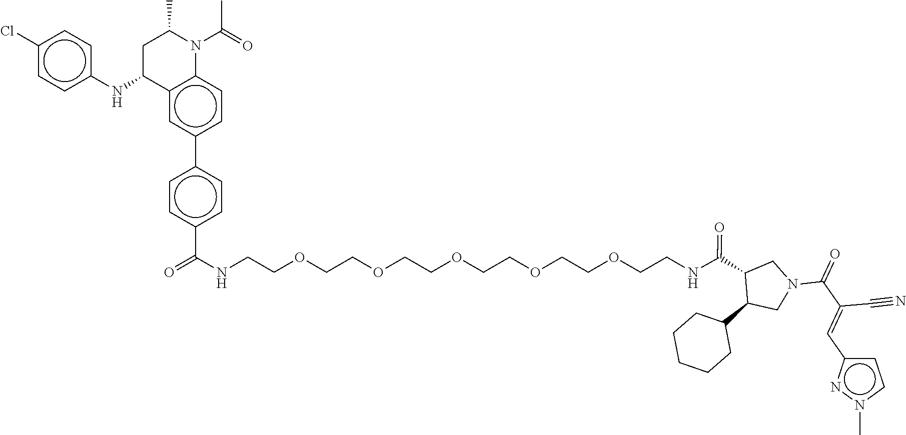 | [M]⁺ 1035.2 and [M + 2]⁺ 1037.2 |
| 15s | 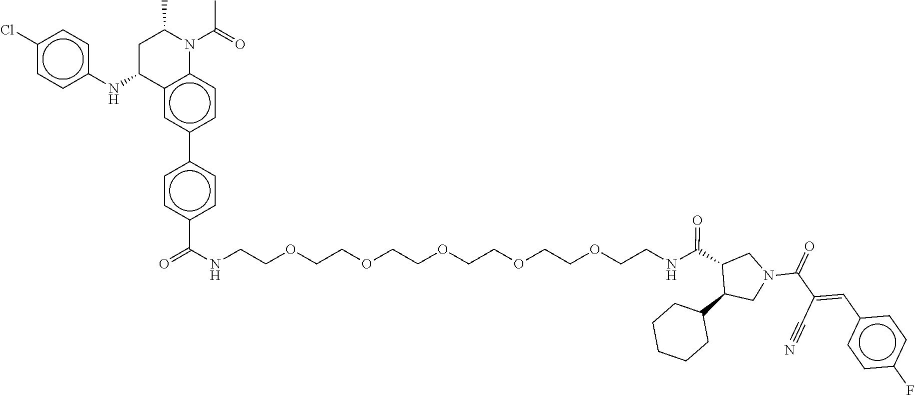 | [M]⁺ 1049.1 and [M + 2]⁺ 1051.1 |
| 15t | 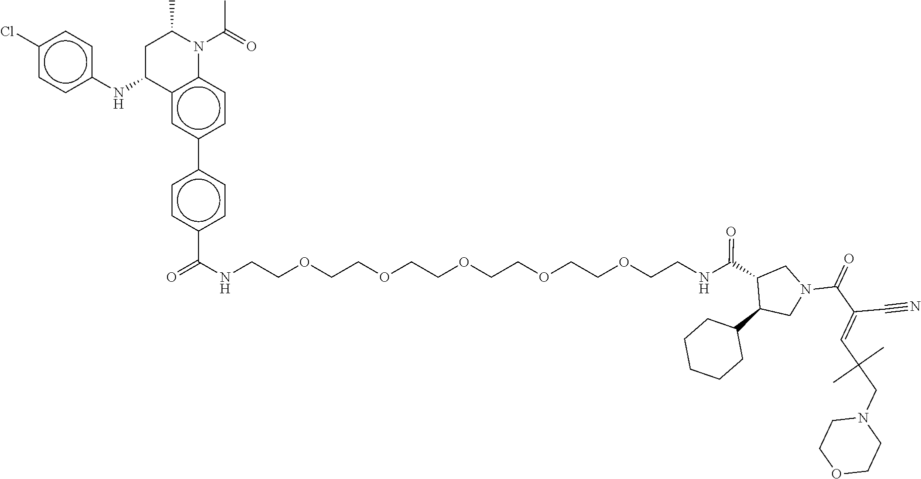 | [M]⁺ 1096.1 and [M + 2]⁺ 1098.1 |
| 15u | 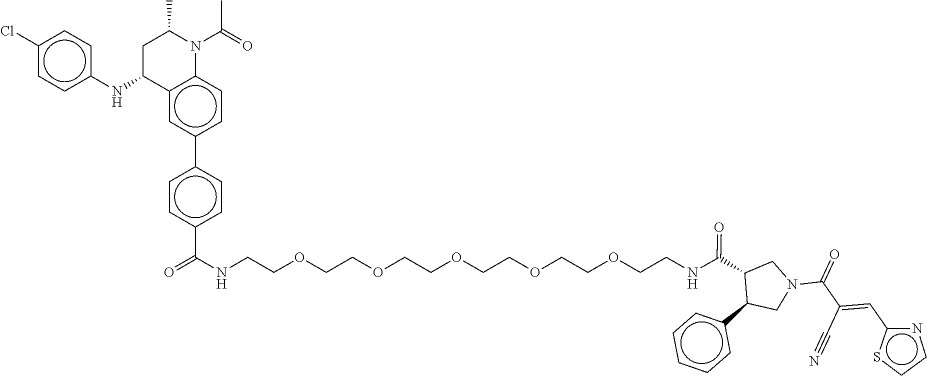 | [M]⁺ 1032.2 and [M + 2]⁺ 1034.2 |
| 15v | 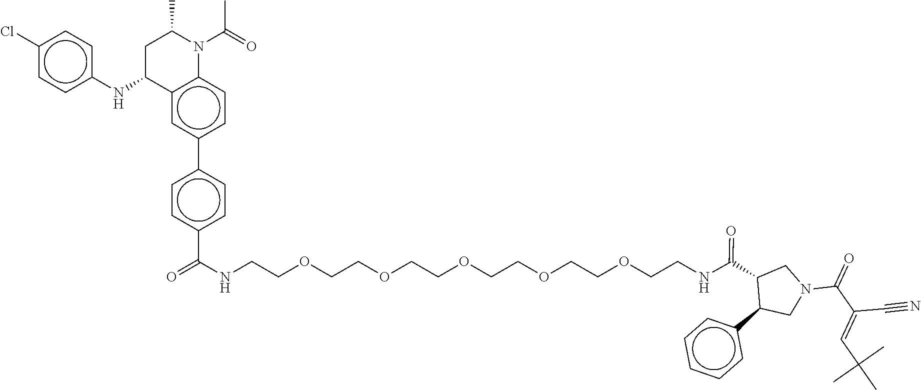 | [M]⁺ 1005.2 and [M + 2]⁺ 1007.2 |
| 15w | 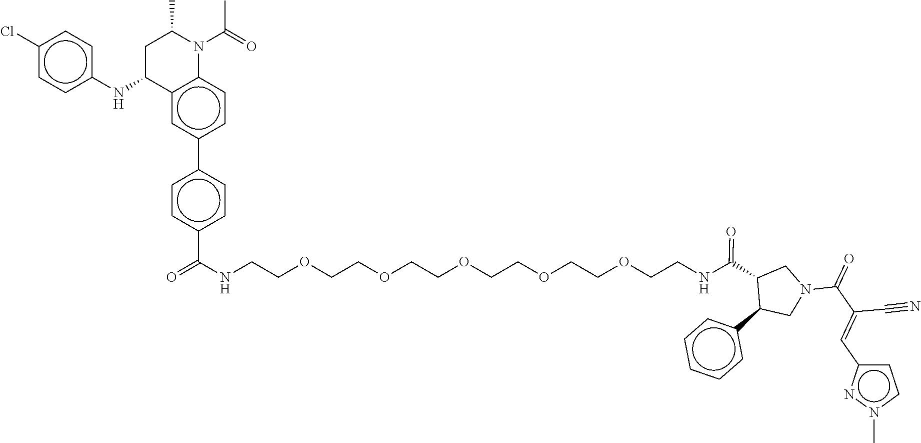 | [M]⁺ 1029.2 and [M + 2]⁺ 1031.2 |
| 15x | 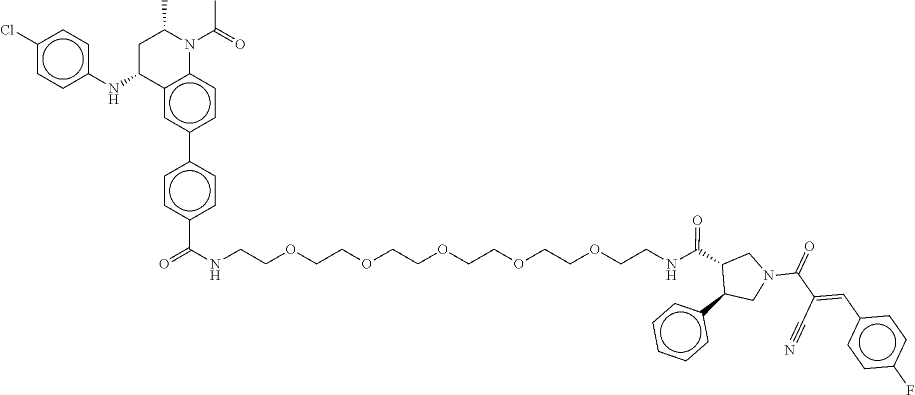 | [M]⁺ 1043.1 and [M + 2]⁺ 1045.1 |
| 15y | 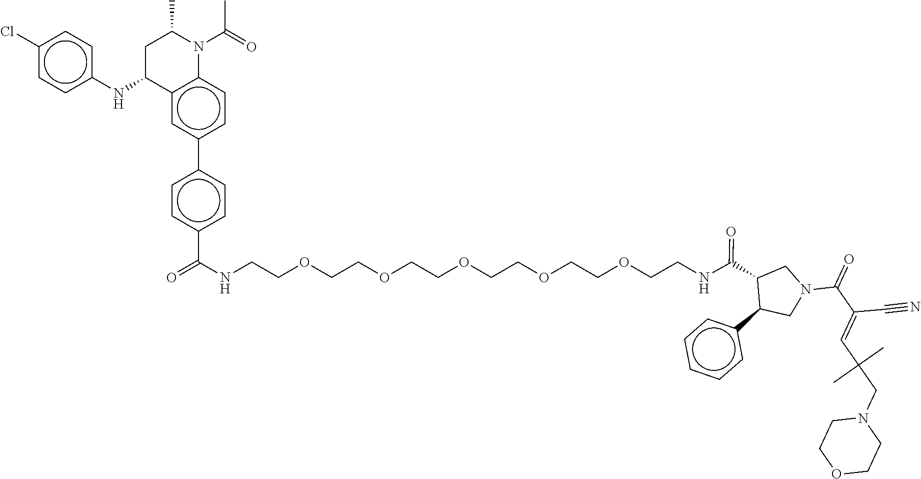 | [M]⁺ 1090.1 and [M + 2]⁺ 1092.1 |
| 15z | 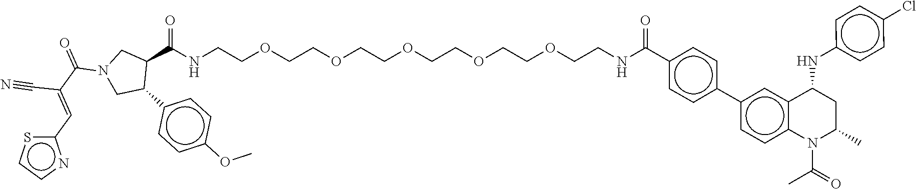 | [M]⁺ 1062.2 and [M + 2]⁺ 1064.2 |
| 15aa | 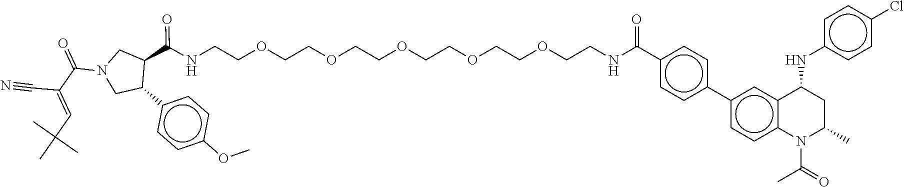 | [M]⁺ 1035.2 and [M + 2]⁺ 1037.2 |
| 15ab | 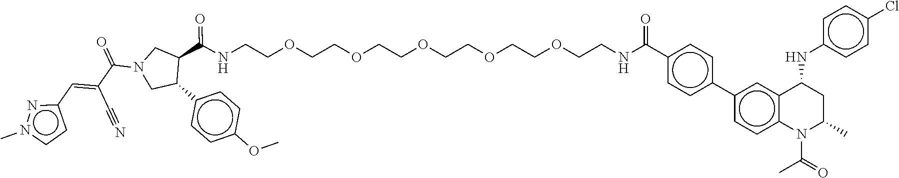 | [M]⁺ 1059.2 and [M + 2]⁺ 1061.2 |
| 15ac | 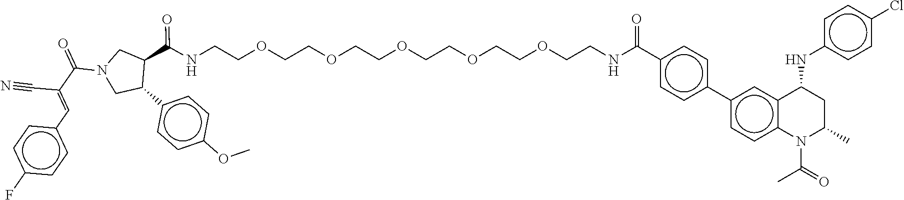 | [M]⁺ 1073.1 and [M + 2]⁺ 1075.1 |
| 15ad | 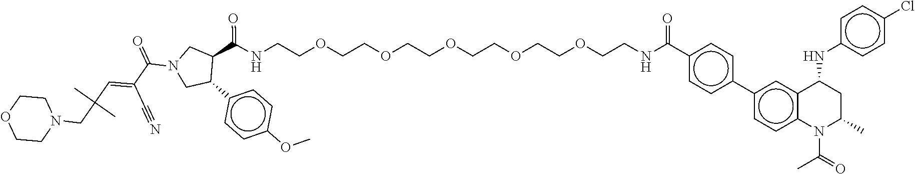 | [M]⁺ 1020.1 and [M + 2]⁺ 1022.1 |
| 15ae | 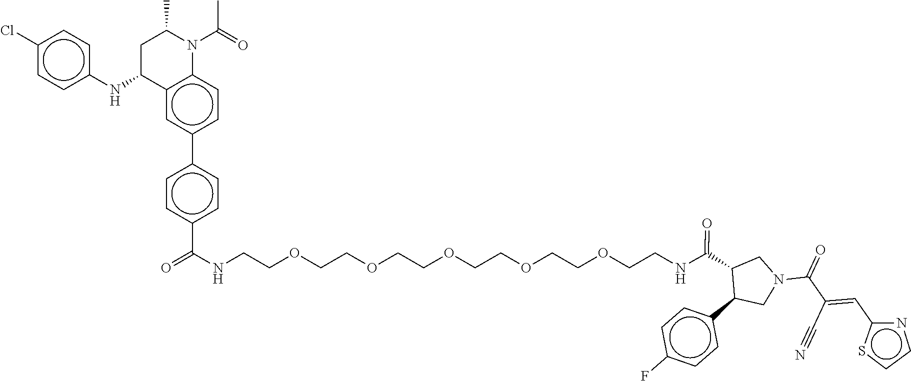 | [M]⁺ 1050.2 and [M + 2]⁺ 1052.2 |
| 15af | 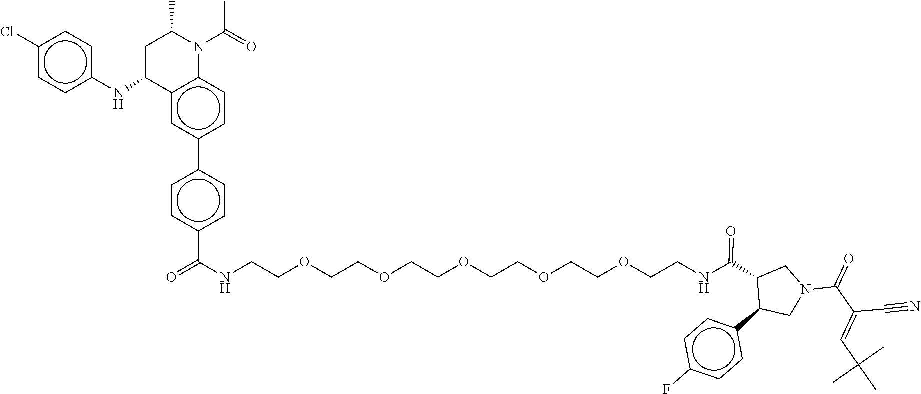 | [M]⁺ 1023.2 and [M + 2]⁺ 1025.2 |
| 15ag | 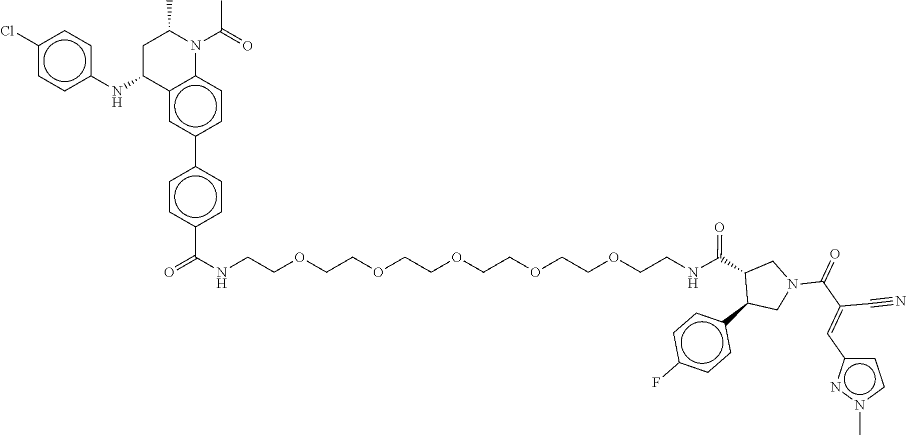 | [M]⁺ 1047.2 and [M + 2]⁺ 1049.2 |
| 15ah | 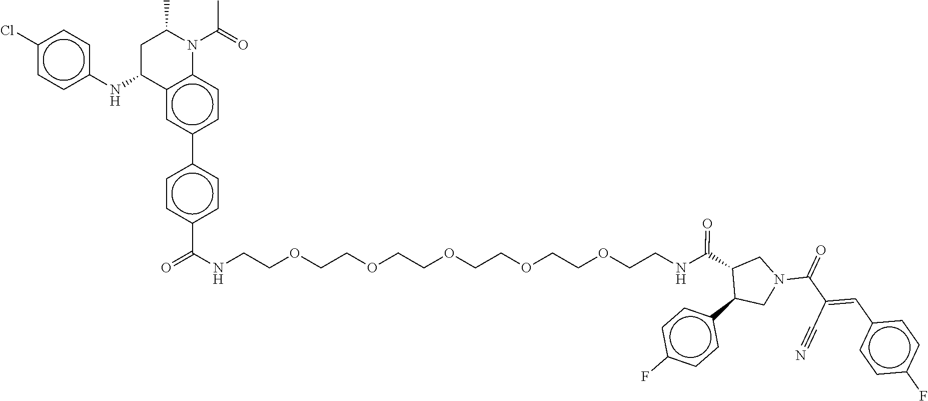 | [M]⁺ 1061.1 and [M + 2]⁺ 1063.1 |
| 15ai | 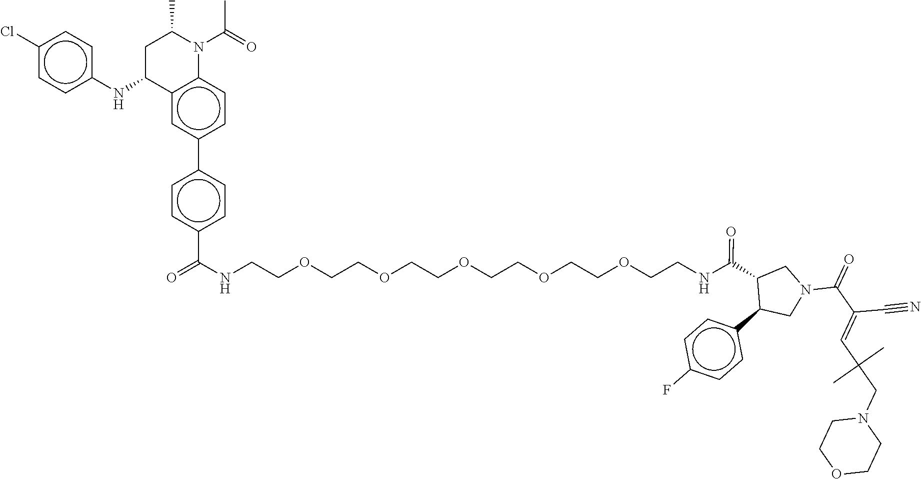 | [M]⁺ 1108.1 and [M + 2]⁺ 1110.1 |
| 15aj | 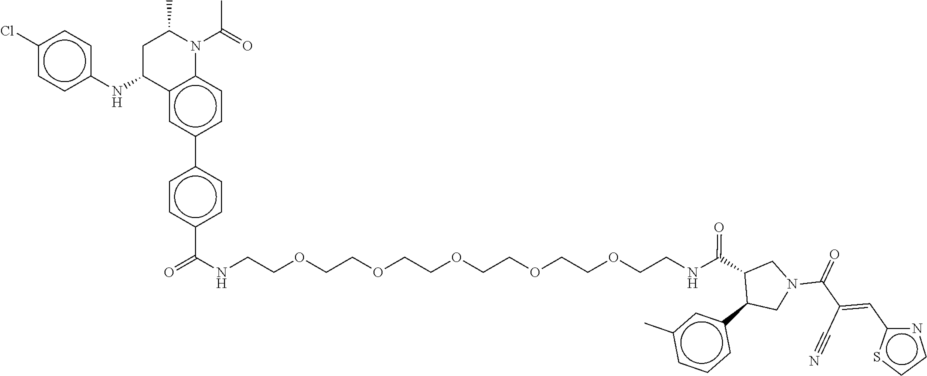 | [M]⁺ 1046.2 and [M + 2]⁺ 1048.2 |
| 15ak | 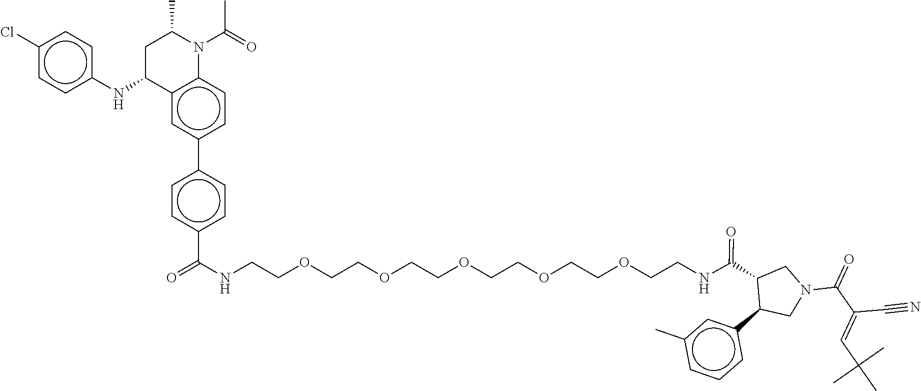 | |
| 15al | 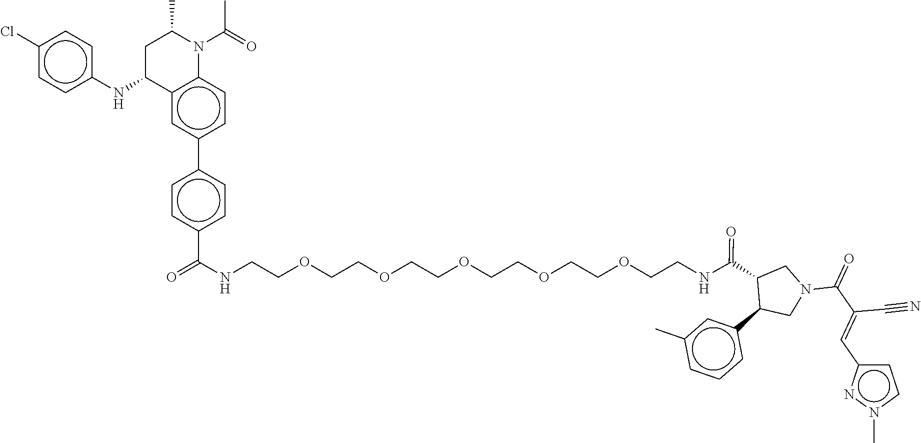 | [M]⁺ 1043.2 and [M + 2]⁺ 1045.2 |
| 15am | 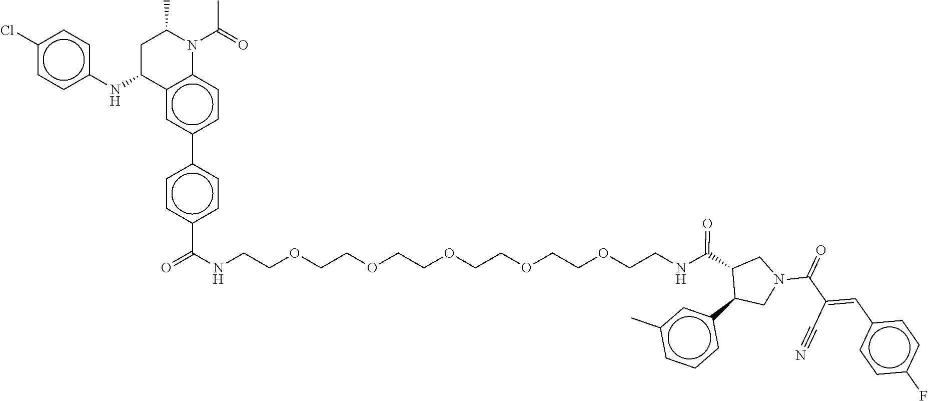 | [M]⁺ 1057.1 and [M + 2]⁺ 1059.1 |
| 15an | 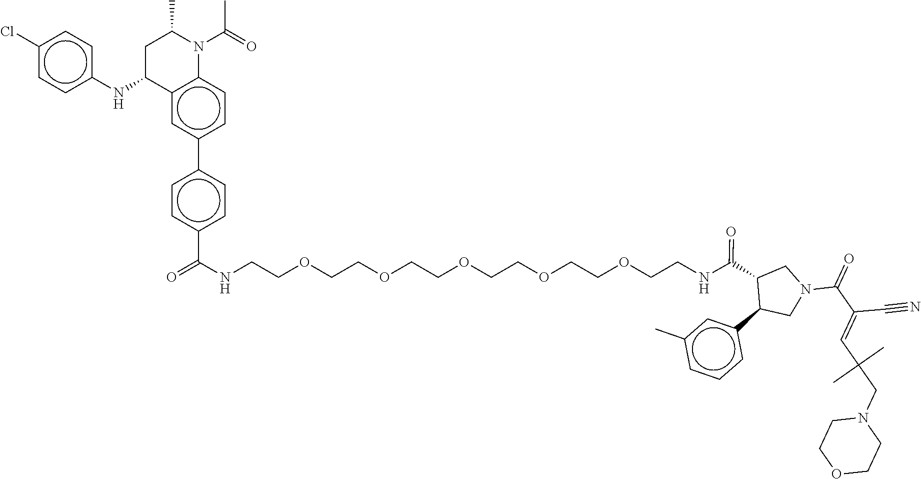 | [M]⁺ 1104.2 and [M + 2]⁺ 1106.2 |

### BRD9 Degraders

Overviews of various exemplary synthetic methods and general procedures that may be used to provide the compounds of the present disclosure are shown below.

### Reductive Amination—General Procedure 1a

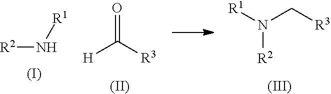

A solution of amine (I) (1.5 equiv.) and aldehyde (II) (1 equiv.) in MeOH (0.05 M) and acetic acid (1.0 equiv.) was stirred for 10 min at room temperature. MP-CNBH₃ (1.0 equiv. w/w) was added and the reaction was stirred for 16 h at 70° C. The reaction mixture was quenched with NaHCO₃ and was extracted with DCM. The combined organic extracts were washed with water, brine, dried over MgSO₄ and concentrated in vacuo. Where required, purification by silica gel column chromatography yielded the desired product.

### Boc Deprotection—General Procedure 2a

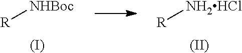

A solution of Boc protected amine (I) (1.0 equiv.) in CH₂Cl₂ (0.05 M) was treated with HCl (4 M in dioxane, 50 equiv.) and the mixture was stirred for 2 h. The volatiles were evaporated in vacuo to yield the corresponding amine hydrochloride (II).

### Amide Coupling—General Procedure 3a

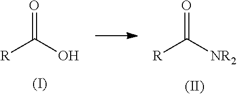

To a stirred solution of carboxylic acid (I) (1.5 equiv.) in DMF was added DIPEA (2.5 equiv.) and HATU (1.5 equiv.). The reaction mixture was stirred for 5 min, then relevant amine (1.0 equiv.) was added and the reaction mixture was stirred for 16 h at RT. The reaction was quenched with ice cold water and extracted with EtOAc. The combined organic layers were concentrated in vacuo to afford the crude product. Where stated, the crude product was purified by silica gel column chromatography/reverse phase preparative HPLC to give the desired amide (II).

### Amide Coupling—General Procedure 3b

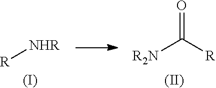

To a stirred solution of amine (I) (1.0 equiv.) and acid (1.0 equiv.) in DMF was added DIPEA (2.0 equiv.) and HATU (1.3 equiv.). The reaction mixture was stirred for 5 min, then relevant amine (1.0 equiv.) were added and the reaction mixture was stirred for 30 mins at RT. The reaction was diluted with methanol and concentrated in vacuo. Where stated, the crude product was purified by silica gel column chromatography/reverse phase preparative HPLC to give the desired amide (II).

### Amine Acylation—General Procedure 4a

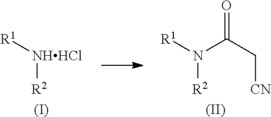

A suspension of amine (I) (1.0 equiv.) in 1,4-dioxane (0.05 M) was treated with Et₃N (4.0 equiv.) and 3-(3,5-dimethyl-1H-pyrazol-1-yl)-3-oxopropanenitrile (1.1 equiv.) and the mixture was heated to 90° C. for 2 h. The volatiles were concentrated in vacuo and purified by flash chromatography to yield the corresponding cyanoacetamide (II).

### Cyano-Knoevenagel Condensation—General Procedure 5a

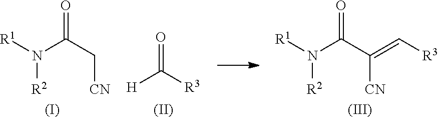

A solution of cyanoacetamide (I) (1.0 equiv.) in THF (0.1 M) was treated with aldehyde (II) (2.5 equiv.) and piperidine (0.5 equiv.) and the mixture was heated to reflux for 72 h. The volatiles were concentrated in vacuo and purified by silica gel column chromatography to yield the corresponding cyanoacrylamide (III).

Cyano-Knoevenagel Condensation—General procedure 6a

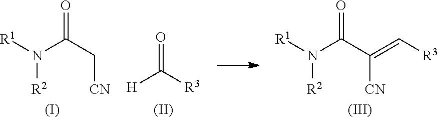

A solution of cyanoacetamide (I) (1.0 equiv.) and aldehyde (II) (4 equiv.) in pyrrolidine (0.6 mL) was treated with acetic acid (1 eq) and the mixture was stirred for 16 h at RT. The volatiles were concentrated in vacuo and purified by silica gel column chromatography to yield the corresponding cyanoacrylamide (III).

### Cyano-Knoevenagel Condensation—General Procedure 7a

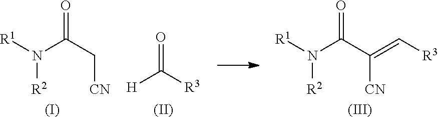

A solution of cyanoacetamide (I) (1.0 equiv.) in Ethanol (0.1 M) was treated with aldehyde (II) (3 equiv.) and piperidine (5 equiv.) and the mixture was heated to 50° C. for 16 h. The volatiles were concentrated in vacuo and purified by silica gel column chromatography to yield the corresponding cyanoacrylamide (III).

Cyano-Knoevenagel Condensation—General procedure 8a

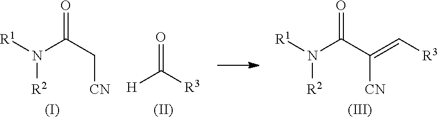

A solution of cyanoacetamide (I) (1.0 equiv.) and aldehyde (II) (4 equiv.) in ethanol: water (2:1, 0.064 M) was treated with beta-alanine (16.0 eq) and the mixture was for 16 h at RT. The volatiles were concentrated in vacuo and purified by silica gel column chromatography to yield the corresponding cyanoacrylamide (III).

### Synthetic Pathways

### Overview of Synthetic Pathway—Scheme 1a

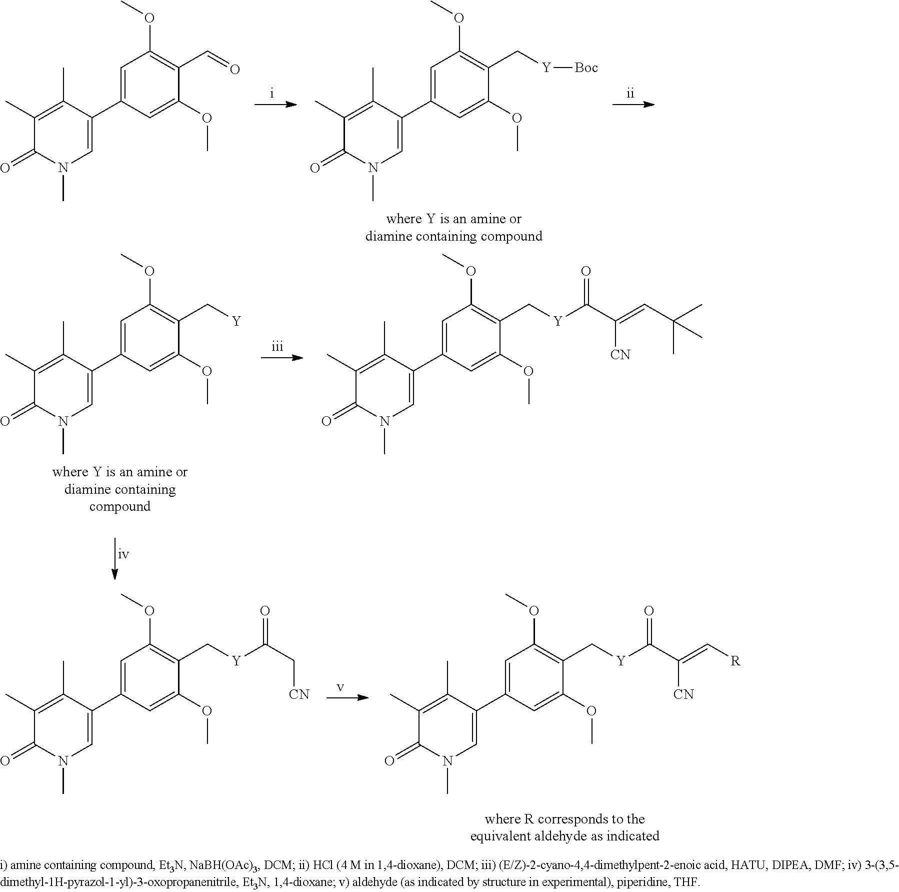

### Overview of Synthetic Pathway—Scheme 2a

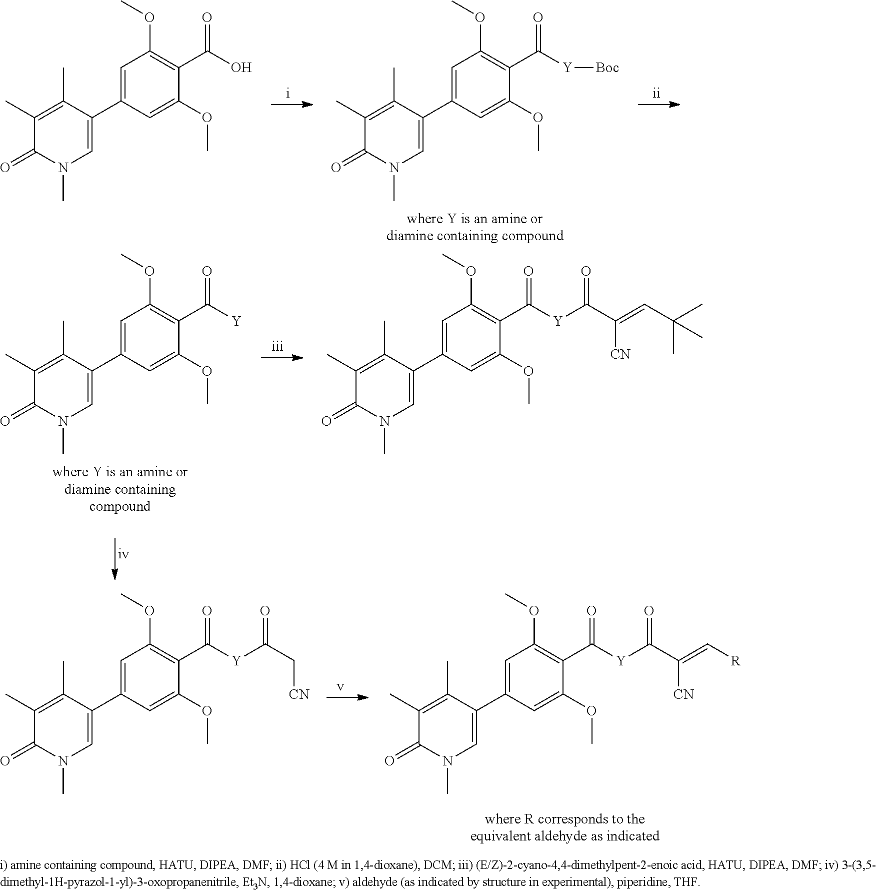

### Overview of Synthetic Pathway—Scheme 3a

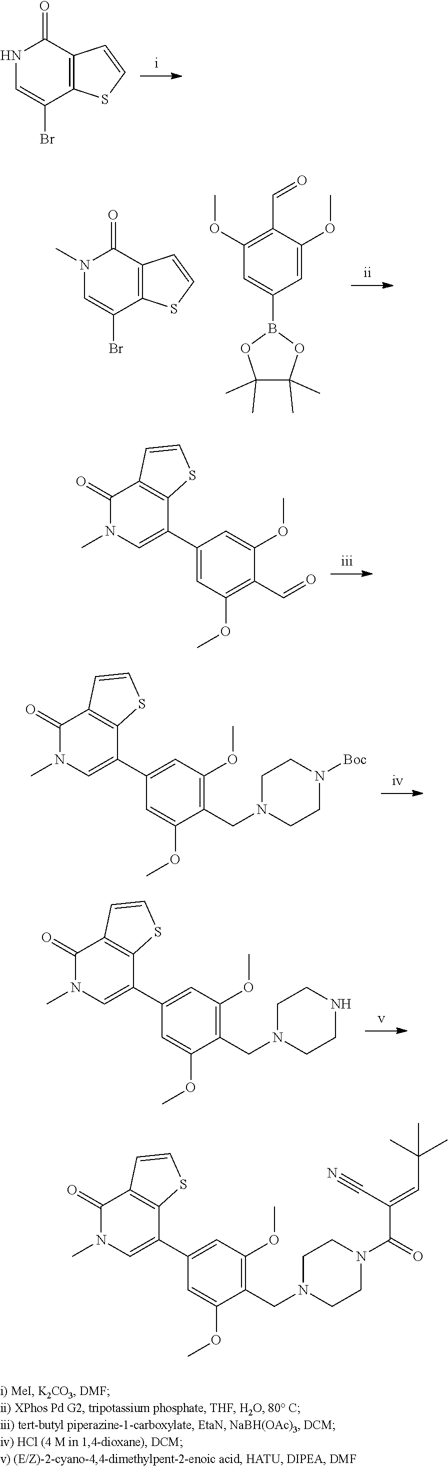

### Preparative Examples

TABLE 1a
The following examples were prepared following general procedure 1a using
aldehyde synthesized according to WO 2021/178920 (compound 172-11 on page 314) and
amine specified.
Table 1a

| | | | LCMS m/z |
|---|---|---|---|
| Name | Amine (Y) | Product Structure | [M + H]⁺/Yield |
| tert-butyl 4-(2,6-dimethoxy-4- (1,4,5-trimethyl-6-oxo-1,6- dihydropyridin-3- yl)benzyl)piperazine-1- carboxylate | 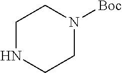 [SMILES: CC(C)(C)OC(=O)N1CCNCC1] | 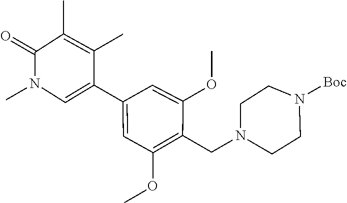 [SMILES: COc1cc(-c2cn(C)c(=O)c(C)c2C)cc(OC)c1CN1CCN(C(=O)OC(C)(C)C)CC1] | 472.2/96% |
| tert-butyl 5-(2,6-dimethoxy-4- (1,4,5-trimethyl-6-oxo-1,6- dihydropyridin-3-yl)benzyl)- 2,5- diazabicyclo[2.2.1]heptane-2- carboxylate | 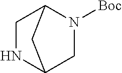 [SMILES: CC(C)(C)OC(=O)N1CC2CC1CN2] | 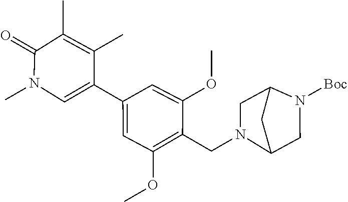 [SMILES: COc1cc(-c2cn(C)c(=O)c(C)c2C)cc(OC)c1CN1CC2CC1CN2C(=O)OC(C)(C)C] | 484.3/95% |
| tert-butyl 3-(2,6-dimethoxy-4- (1,4,5-trimethyl-6-oxo-1,6- dihydropyridin-3-yl)benzyl)- 3,6- diazabicyclo[3.1.1]heptane-6- carboxylate | 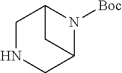 [SMILES: CC(C)(C)OC(=O)N1C2CNCC1C2] | 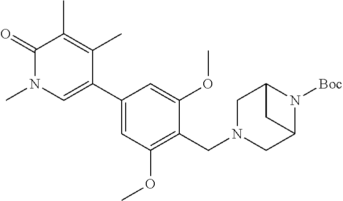 [SMILES: COc1cc(-c2cn(C)c(=O)c(C)c2C)cc(OC)c1CN1CC2CC(C1)N2C(=O)OC(C)(C)C] | 484.5/95% |
| tert-butyl 6-(2,6-dimethoxy-4- (1,4,5-trimethyl-6-oxo-1,6- dihydropyridin-3-yl)benzyl)- 3,6- diazabicyclo[3.1.1]heptane-3- carboxylate | 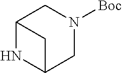 [SMILES: CC(C)(C)OC(=O)N1CC2CC(C1)N2] | 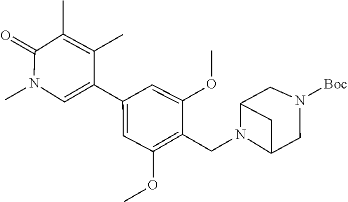 [SMILES: COc1cc(-c2cn(C)c(=O)c(C)c2C)cc(OC)c1CN1C2CC1CN(C(=O)OC(C)(C)C)C2] | 484.3/92% |
| tert-butyl 3-(2,6-dimethoxy-4- (1,4,5-trimethyl-6-oxo-1,6- dihydropyridin-3-yl)benzyl)- 3,8-diazabicyclo[3.2.1]octane- 8-carboxylate | 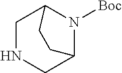 [SMILES: CC(C)(C)OC(=O)N1C2CCC1CNC2] | 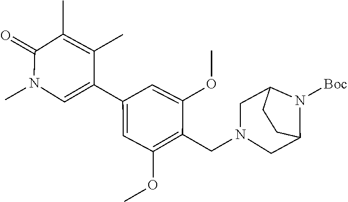 [SMILES: COc1cc(-c2cn(C)c(=O)c(C)c2C)cc(OC)c1CN1CC2CCC(C1)N2C(=O)OC(C)(C)C] | 498.4/91% |
| tert-butyl 4-(2,6-dimethoxy-4- (1,4,5-trimethyl-6-oxo-1,6- dihydropyridin-3-yl)benzyl)- 1,4-diazepane-1-carboxylate | 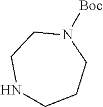 [SMILES: CC(C)(C)OC(=O)N1CCCNCC1] | 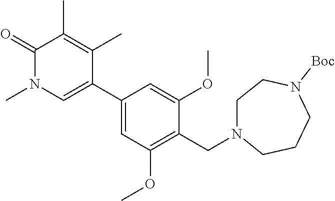 [SMILES: COc1cc(-c2cn(C)c(=O)c(C)c2C)cc(OC)c1CN1CCCN(C(=O)OC(C)(C)C)CC1] | 486.4/76% |
| tert-butyl 4-((2,6-dimethoxy-4- (1,4,5-trimethyl-6-oxo-1,6- dihydropyridin-3- yl)benzyl)(methyl)amino) piperidine-1-carboxylate | 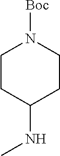 | 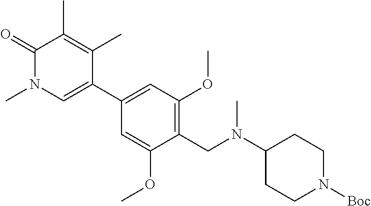 [SMILES: COc1cc(-c2cn(C)c(=O)c(C)c2C)cc(OC)c1CN(C)C1CCN(C(=O)OC(C)(C)C)CC1] | 500.2/97% |
| tert-butyl 7-(2,6-dimethoxy-4- (1,4,5-trimethyl-6-oxo-1,6- dihydropyridin-3-yl)benzyl)- 4,7-diazaspiro[2.5]octane-4- carboxylate | 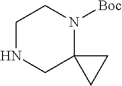 [SMILES: CC(C)(C)OC(=O)N1CCNCC12CC2] | 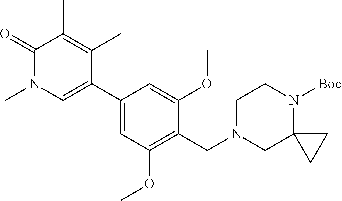 [SMILES: COc1cc(-c2cn(C)c(=O)c(C)c2C)cc(OC)c1CN1CCN(C(=O)OC(C)(C)C)C2(CC2)C1] | 498.3/95% |
| tert-butyl 4-(2,6-dimethoxy-4- (1,4,5-trimethyl-6-oxo-1,6- dihydropyridin-3-yl)benzyl)- 4,7-diazaspiro[2.5]octane-7- carboxylate | 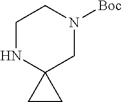 [SMILES: CC(C)(C)OC(=O)N1CCNC2(CC2)C1] | 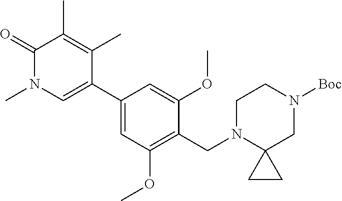 [SMILES: COc1cc(-c2cn(C)c(=O)c(C)c2C)cc(OC)c1CN1CCN(C(=O)OC(C)(C)C)CC12CC2] | 498.4/94% |
| tert-butyl 6-(2,6-dimethoxy-4- (1,4,5-trimethyl-6-oxo-1,6- dihydropyridin-3-yl)benzyl)- 2,6-diazaspiro[3.3]heptane-2- carboxylate | 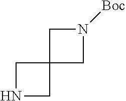 [SMILES: CC(C)(C)OC(=O)N1CC2(CNC2)C1] | 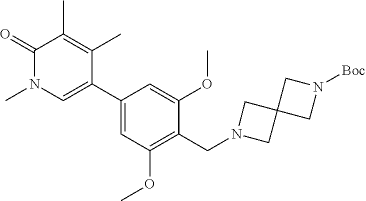 [SMILES: COc1cc(-c2cn(C)c(=O)c(C)c2C)cc(OC)c1CN1CC2(C1)CN(C(=O)OC(C)(C)C)C2] | 484.2/99% |
| tert-butyl 9-(2,6-dimethoxy-4- (1,4,5-trimethyl-6-oxo-1,6- dihydropyridin-3-yl)benzyl)- 3,9-diazaspiro[5.5]undecane-3- carboxylate | 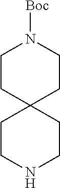 [SMILES: CC(C)(C)OC(=O)N1CCC2(CCNCC2)CC1] | 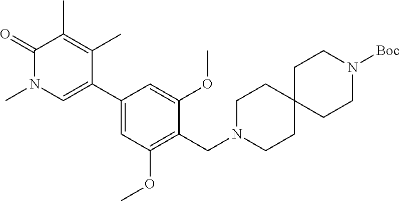 [SMILES: COc1cc(-c2cn(C)c(=O)c(C)c2C)cc(OC)c1CN1CCC2(CC1)CCN(C(=O)OC(C)(C)C)CC2] | 540.5/85% |
| tert-butyl (2R,5S)-4-(2,6- dimethoxy-4-(1,4,5-trimethyl- 6-oxo-1,6-dihydropyridin-3- yl)benzyl)-2,5- dimethylpiperazine-1- carboxylate | 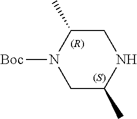 | 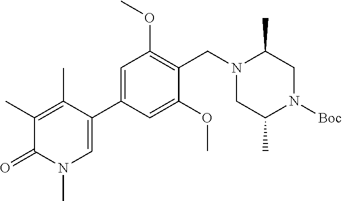 [SMILES: COc1cc(-c2cn(C)c(=O)c(C)c2C)cc(OC)c1CN1C[C@@H](C)N(C(=O)OC(C)(C)C)C[C@@H]1C] | 500.3/98% |

TABLE 2a
The following examples were prepared following general procedure 2a using the
starting materials synthesized in Table 1a.
Table 2a

| | | LCMS m/z |
|---|---|---|
| Name | Product Structure | [M + H]⁺/Yield |
| 5-(3,5-dimethoxy-4-(piperazin-1- ylmethyl)phenyl)-1,3,4- trimethylpyridin-2(1H)-one | 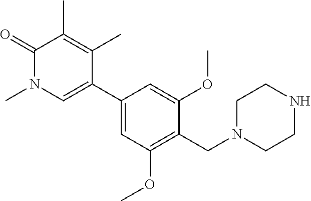 [SMILES: COc1cc(-c2cn(C)c(=O)c(C)c2C)cc(OC)c1CN1CCNCC1] | 372.4/94% |
| 5-(4-((2,5- diazabicyclo[2.2.1]heptan-2- yl)methyl)-3,5- dimethoxyphenyl)-1,3,4- trimethylpyridin-2(1H)-one | 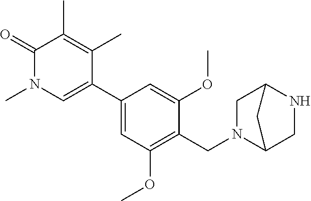 [SMILES: COc1cc(-c2cn(C)c(=O)c(C)c2C)cc(OC)c1CN1CC2CC1CN2] | 384.3/94% |
| 5-(4-((3,6- diazabicyclo[3.1.1]heptan-3- yl)methyl)-3,5- dimethoxyphenyl)-1,3,4- trimethylpyridin-2(1H)-one | 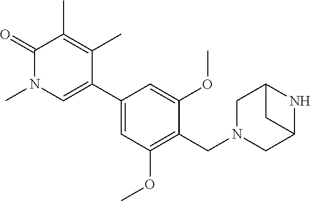 [SMILES: COc1cc(-c2cn(C)c(=O)c(C)c2C)cc(OC)c1CN1CC2CC(C1)N2] | 384.4/87% |
| 5-(4-((3,6- diazabicyclo[3.1.1]heptan-6- yl)methyl)-3,5- dimethoxyphenyl)-1,3,4- trimethylpyridin-2(1H)-one | 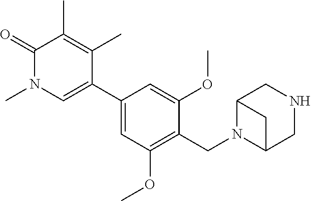 [SMILES: COc1cc(-c2cn(C)c(=O)c(C)c2C)cc(OC)c1CN1C2CNCC1C2] | 384.4/94% |
| 5-(4-((3,8- diazabicyclo[3.2.1]octan-3- yl)methyl)-3,5- dimethoxyphenyl)-1,3,4- trimethylpyridin-2(1H)-one | 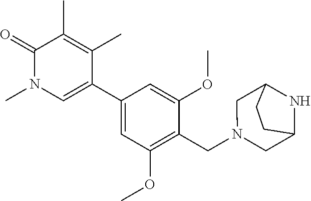 [SMILES: COc1cc(-c2cn(C)c(=O)c(C)c2C)cc(OC)c1CN1CC2CCC(C1)N2] | 398.5/87% |
| 5-(4-((1,4-diazepan-1-yl)methyl)- 3,5-dimethoxyphenyl)-1,3,4- trimethylpyridin-2(1H)-one | 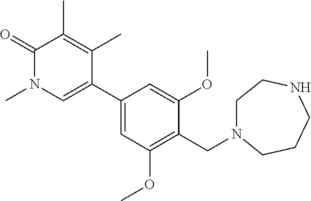 [SMILES: COc1cc(-c2cn(C)c(=O)c(C)c2C)cc(OC)c1CN1CCCNCC1] | 386.3/92% |
| 5-(3,5-dimethoxy-4- ((methyl(piperidin-4- yl)amino)methyl)phenyl)-1,3,4- trimethylpyridin-2(1H)-one | 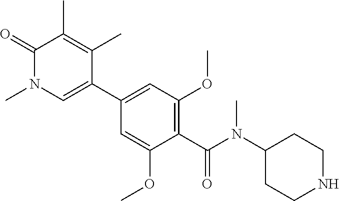 [SMILES: COc1cc(-c2cn(C)c(=O)c(C)c2C)cc(OC)c1C(=O)N(C)C1CCNCC1] | 400.2/94% |
| 5-(4-((4,7-diazaspiro[2.5]octan-7- yl)methyl)-3,5- dimethoxyphenyl)-1,3,4- trimethylpyridin-2(1H)-one | 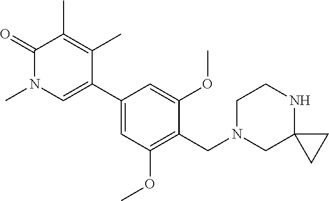 [SMILES: COc1cc(-c2cn(C)c(=O)c(C)c2C)cc(OC)c1CN1CCNC2(CC2)C1] | 398.3/93% |
| 5-(4-((4,7-diazaspiro[2.5]octan-4- yl)methyl)-3,5- dimethoxyphenyl)-1,3,4- trimethylpyridin-2(1H)-one | 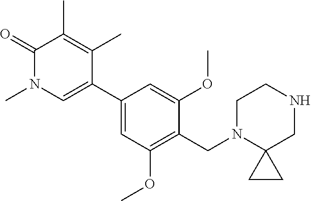 [SMILES: COc1cc(-c2cn(C)c(=O)c(C)c2C)cc(OC)c1CN1CCNCC12CC2] | 398.4/99% |
| 5-(4-((2,6-diazaspiro[3.3]heptan- 2-yl)methyl)-3,5- dimethoxyphenyl)-1,3,4- trimethylpyridin-2(1H)-one | 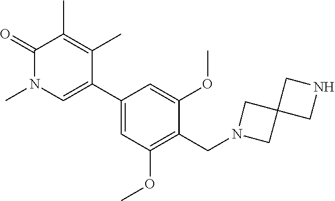 [SMILES: COc1cc(-c2cn(C)c(=O)c(C)c2C)cc(OC)c1CN1CC2(CNC2)C1] | 384.2/99% (using TFA instead of HCl) |
| 5-(4-((3,9- diazaspiro[5.5]undecan-3- yl)methyl)-3,5- dimethoxyphenyl)-1,3,4- trimethylpyridin-2(1H)-one | 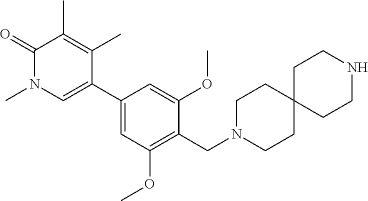 [SMILES: COc1cc(-c2cn(C)c(=O)c(C)c2C)cc(OC)c1CN1CCC2(CCNCC2)CC1] | 440.5/91% |
| 5-(4-(((2S,5R)-2,5- dimethylpiperazin-1-yl)methyl)- 3,5-dimethoxyphenyl)-1,3,4- trimethylpyridin-2(1H)-one | 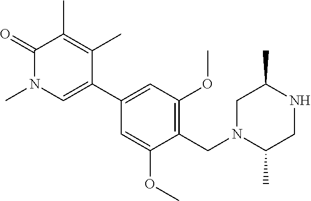 [SMILES: COc1cc(-c2cn(C)c(=O)c(C)c2C)cc(OC)c1CN1C[C@@H](C)NC[C@@H]1C] | 400.2/ not isolated |

TABLE 3a
The following examples were prepared following general procedure 3a using starting
materials synthesized in Table 2a and (E/Z)-2-cyano-4,4-dimethylpent-2-enoic acid.
Table 3a

| | | LCMS m/z |
|---|---|---|
| Name | Product Structure/Name | [M + H]⁺/Yield |
| BRD9a | 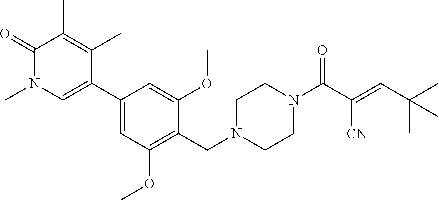 [SMILES: COc1cc(-c2cn(C)c(=O)c(C)c2C)cc(OC)c1CN1CCN(C(=O)/C(C#N)=C/C(C)(C)C)CC1] | 507.2/48% |
| | (E/Z)-2-(4-(2,6-dimethoxy-4-(1,4,5-trimethyl- | |
| | 6-oxo-1,6-dihydropyridin-3- | |
| | yl)benzyl)piperazine-1-carbonyl)-4,4- | |
| | dimethylpent-2-enenitrile | |
| BRD9b | 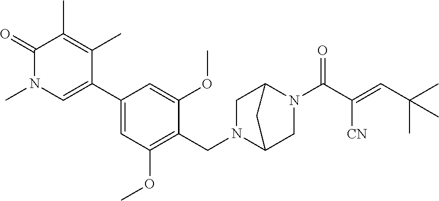 [SMILES: COc1cc(-c2cn(C)c(=O)c(C)c2C)cc(OC)c1CN1CC2CC1CN2C(=O)/C(C#N)=C/C(C)(C)C] | 519.2/27% |
| | (E/Z)-2-(5-(2,6-dimethoxy-4-(1,4,5-trimethyl- | |
| | 6-oxo-1,6-dihydropyridin-3-yl)benzyl)-2,5- | |
| | diazabicyclo[2.2.1]heptane-2-carbonyl)-4,4- | |
| | dimethylpent-2-enenitrile | |
| BRD9c | 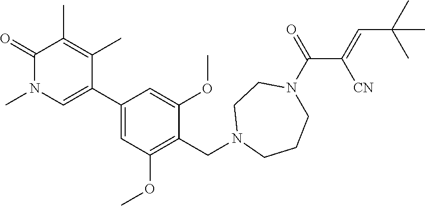 [SMILES: COc1cc(-c2cn(C)c(=O)c(C)c2C)cc(OC)c1CN1CCCN(C(=O)/C(C#N)=C/C(C)(C)C)CC1] | 521.2/48% |
| | (E/Z)-2-(4-(2,6-dimethoxy-4-(1,4,5-trimethyl- | |
| | 6-oxo-1,6-dihydropyridin-3-yl)benzyl)-1,4- | |
| | diazepane-1-carbonyl)-4,4-dimethylpent-2- | |
| | enenitrile | |
| BRD9d | 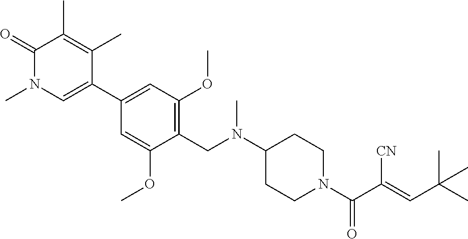 [SMILES: COc1cc(-c2cn(C)c(=O)c(C)c2C)cc(OC)c1CN(C)C1CCN(C(=O)/C(C#N)=C/C(C)(C)C)CC1] | 535.6/24% |
| | (E/Z)-2-(4-((2,6-dimethoxy-4-(1,4,5-trimethyl- | |
| | 6-oxo-1,6-dihydropyridin-3- | |
| | yl)benzyl)(methyl)amino)piperidine-1- | |
| | carbonyl)-4,4-dimethylpent-2-enenitrile | |
| BRD9e | 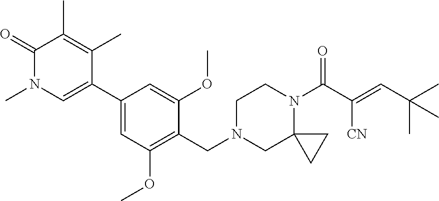 [SMILES: COc1cc(-c2cn(C)c(=O)c(C)c2C)cc(OC)c1CN1CCN(C(=O)/C(C#N)=C/C(C)(C)C)C2(CC2)C1] | 533.2/49% |
| | (E/Z)-2-(7-(2,6-dimethoxy-4-(1,4,5-trimethyl- | |
| | 6-oxo-1,6-dihydropyridin-3-yl)benzyl)-4,7- | |
| | diazaspiro[2.5]octane-4-carbonyl)-4,4- | |
| | dimethylpent-2-enenitrile | |
| BRD9f | 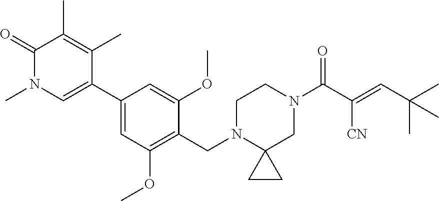 [SMILES: COc1cc(-c2cn(C)c(=O)c(C)c2C)cc(OC)c1CN1CCN(C(=O)/C(C#N)=C/C(C)(C)C)CC12CC2] | 533.3/35% |
| | (E/Z)-2-(4-(2,6-dimethoxy-4-(1,4,5-trimethyl- | |
| | 6-oxo-1,6-dihydropyridin-3-yl)benzyl)-4,7- | |
| | diazaspiro[2.5]octane-7-carbonyl)-4,4- | |
| | dimethylpent-2-enenitrile | |
| BRD9ad | 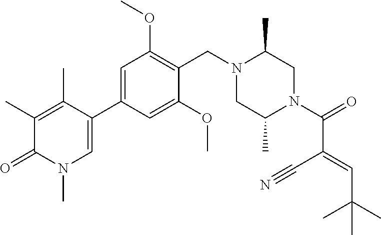 [SMILES: COc1cc(-c2cn(C)c(=O)c(C)c2C)cc(OC)c1CN1C[C@@H](C)N(C(=O)/C(C#N)=C/C(C)(C)C)C[C@@H]1C] | 535.3/18% |
| | (E)-2-((2R,5S)-4-(2,6-dimethoxy-4-(1,4,5- | |
| | trimethyl-6-oxo-1,6-dihydropyridin-3- | |
| | yl)benzyl)-2,5-dimethylpiperazine-1- | |
| | carbonyl)-4,4-dimethylpent-2-enenitrile | |

TABLE 4a
The following examples were prepared following general procedure 4a using
starting materials synthesized in Table 2a.
Table 4a

| | | LCMS m/z |
|---|---|---|
| Name | Product Structure | [M + H]⁺/Yield |
| 3-(4-(2,6-dimethoxy-4-(1,4,5- trimethyl-6-oxo-1,6- dihydropyridin-3- yl)benzyl)piperazin-1-yl)-3- oxopropanenitrile | 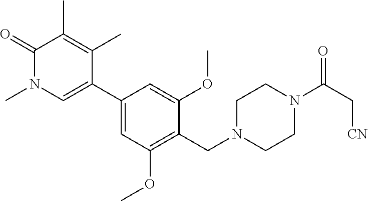 [SMILES: COc1cc(-c2cn(C)c(=O)c(C)c2C)cc(OC)c1CN1CCN(C(=O)CC#N)CC1] | 439.3/40% |
| 3-(5-(2,6-dimethoxy-4-(1,4,5- trimethyl-6-oxo-1,6- dihydropyridin-3-yl)benzyl)-2,5- diazabicyclo[2.2.1]heptan-2-yl)-3- oxopropanenitrile | 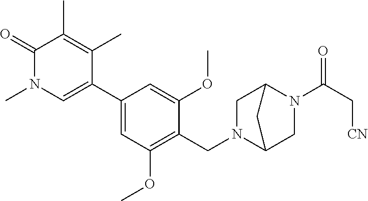 [SMILES: COc1cc(-c2cn(C)c(=O)c(C)c2C)cc(OC)c1CN1CC2CC1CN2C(=O)CC#N] | 451.4/62% |
| 3-(3-(2,6-dimethoxy-4-(1,4,5- trimethyl-6-oxo-1,6- dihydropyridin-3-yl)benzyl)-3,6- diazabicyclo[3.1.1]heptan-6-yl)-3- oxopropanenitrile | 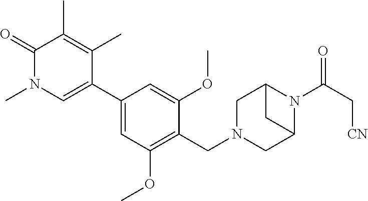 [SMILES: COc1cc(-c2cn(C)c(=O)c(C)c2C)cc(OC)c1CN1CC2CC(C1)N2C(=O)CC#N] | 451.5/60% |
| 3-(6-(2,6-dimethoxy-4-(1,4,5- trimethyl-6-oxo-1,6- dihydropyridin-3-yl)benzyl)-3,6- diazabicyclo[3.1.1]heptan-3-yl)-3- oxopropanenitrile | 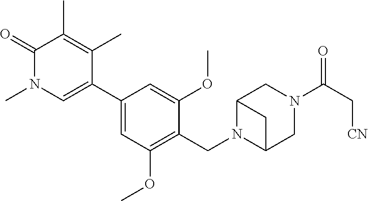 [SMILES: COc1cc(-c2cn(C)c(=O)c(C)c2C)cc(OC)c1CN1C2CC1CN(C(=O)CC#N)C2] | 451.4/49% |
| 3-(3-(2,6-dimethoxy-4-(1,4,5- trimethyl-6-oxo-1,6- dihydropyridin-3-yl)benzyl)-3,8- diazabicyclo[3.2.1]octan-8-yl)-3- oxopropanenitrile | 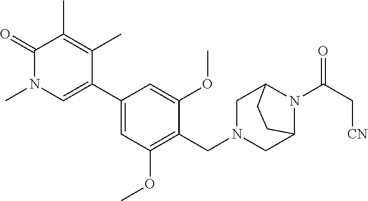 [SMILES: COc1cc(-c2cn(C)c(=O)c(C)c2C)cc(OC)c1CN1CC2CCC(C1)N2C(=O)CC#N] | 465.2/11% |
| 3-(4-((2,6-dimethoxy-4-(1,4,5- trimethyl-6-oxo-1,6- dihydropyridin-3- yl)benzyl)(methyl)amino)piperidin- 1-yl)-3-oxopropanenitrile | 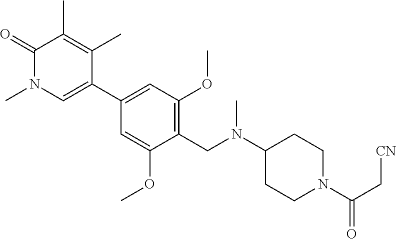 [SMILES: COc1cc(-c2cn(C)c(=O)c(C)c2C)cc(OC)c1CN(C)C1CCN(C(=O)CC#N)CC1] | 467.2/68% |
| 3-(9-(2,6-dimethoxy-4-(1,4,5- trimethyl-6-oxo-1,6- dihydropyridin-3-yl)benzyl)-3,9- diazaspiro[5.5]undecan-3-yl)-3- oxopropanenitrile | 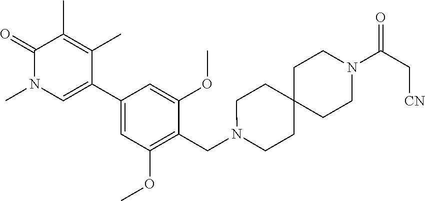 [SMILES: COc1cc(-c2cn(C)c(=O)c(C)c2C)cc(OC)c1CN1CCC2(CC1)CCN(C(=O)CC#N)CC2] | 507.4/45% |

TABLE 5a
The following examples were prepared following general procedure 5a using
starting materials synthesized in Table 4a.
Table 5a

| | | LCMS m/z |
|---|---|---|
| Name | Product Structure/Name | [M + H]⁺/Yield |
| BRD9g | 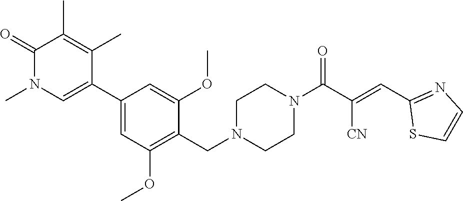 [SMILES: COc1cc(-c2cn(C)c(=O)c(C)c2C)cc(OC)c1CN1CCN(C(=O)/C(C#N)=C/c2nccs2)CC1] | 592.2/18% |
| | (E/Z)-2-(4-(2,6-dimethoxy-4-(1,4,5-trimethyl-6-oxo-1,6- | |
| | dihydropyridin-3-yl)benzyl)piperazine-1-carbonyl)-3-(thiazol- | |
| | 2-yl)acrylonitrile | |
| BRD9h | 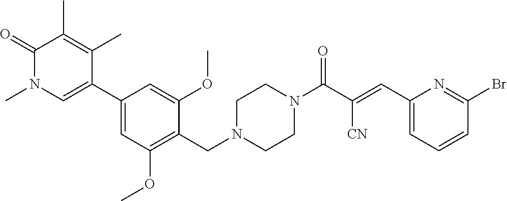 [SMILES: COc1cc(-c2cn(C)c(=O)c(C)c2C)cc(OC)c1CN1CCN(C(=O)/C(C#N)=C/c2cccc(Br)n2)CC1] | 608.2/14% |
| | (E/Z)-3-(6-bromopyridin-2-yl)-2-(4-(2,6-dimethoxy-4-(1,4,5- | |
| | trimethyl-6-oxo-1,6-dihydropyridin-3-yl)benzyl)piperazine-1- | |
| | carbonyl)acrylonitrile | |
| BRD9i | 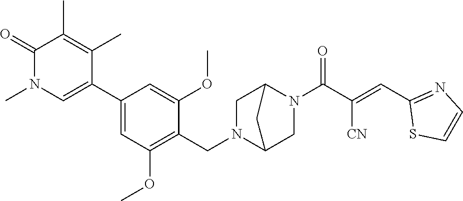 [SMILES: COc1cc(-c2cn(C)c(=O)c(C)c2C)cc(OC)c1CN1CC2CC1CN2C(=O)/C(C#N)=C/c1nccs1] | 546.2/13% |
| | (E/Z)-2-(5-(2,6-dimethoxy-4-(1,4,5-trimethyl-6-oxo-1,6- | |
| | dihydropyridin-3-yl)benzyl)-2,5-diazabicyclo[2.2.1]heptane- | |
| | 2-carbonyl)-3-(thiazol-2-yl)acrylonitrile | |
| BRD9j | 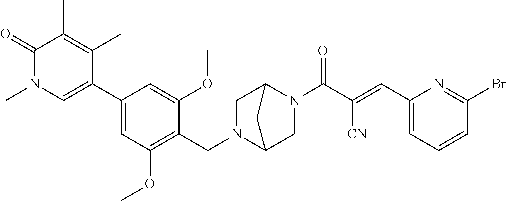 [SMILES: COc1cc(-c2cn(C)c(=O)c(C)c2C)cc(OC)c1CN1CC2CC1CN2C(=O)/C(C#N)=C/c1cccc(Br)n1] | 619.4/12% |
| | (E/Z)-3-(6-bromopyridin-2-yl)-2-(5-(2,6-dimethoxy-4-(1,4,5- | |
| | trimethyl-6-oxo-1,6-dihydropyridin-3-yl)benzyl)-2,5- | |
| | diazabicyclo[2.2.1]heptane-2-carbonyl)acrylonitrile | |
| BRD9k | 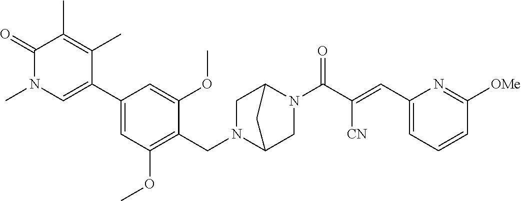 [SMILES: COc1cccc(/C=C(\C#N)C(=O)N2CC3CC2CN3Cc2c(OC)cc(-c3cn(C)c(=O)c(C)c3C)cc2OC)n1] | 570.5/16% |
| | (E/Z)-2-(5-(2,6-dimethoxy-4-(1,4,5-trimethyl-6-oxo-1,6- | |
| | dihydropyridin-3-yl)benzyl)-2,5-diazabicyclo[2.2.1]heptane- | |
| | 2-carbonyl)-3-(6-methoxypyridin-2-yl)acrylonitrile | |
| BRD9x | 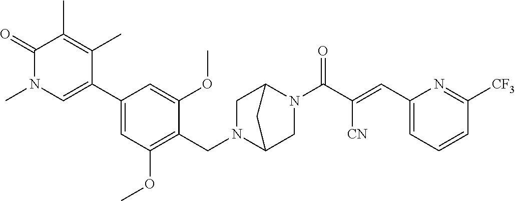 [SMILES: COc1cc(-c2cn(C)c(=O)c(C)c2C)cc(OC)c1CN1CC2CC1CN2C(=O)/C(C#N)=C/c1cccc(C(F)(F)F)n1] | 608.5/12% |
| | (E/Z)-2-(5-(2,6-dimethoxy-4-(1,4,5-trimethyl-6-oxo-1,6- | |
| | dihydropyridin-3-yl)benzyl)-2,5-diazabicyclo[2.2.1]heptane- | |
| | 2-carbonyl)-3-(6-(trifluoromethyl)pyridin-2-yl)acrylonitrile | |
| BRD9l | 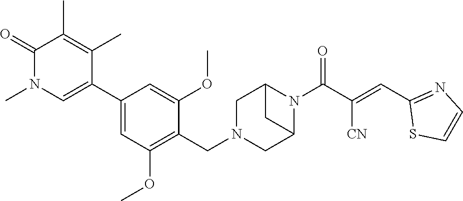 [SMILES: COc1cc(-c2cn(C)c(=O)c(C)c2C)cc(OC)c1CN1CC2CC(C1)N2C(=O)/C(C#N)=C/c1nccs1] | 546.2/18% |
| | (E/Z)-2-(3-(2,6-dimethoxy-4-(1,4,5-trimethyl-6-oxo-1,6- | |
| | dihydropyridin-3-yl)benzyl)-3,6-diazabicyclo[3.1.1]heptane- | |
| | 6-carbonyl)-3-(thiazol-2-yl)acrylonitrile | |
| BRD9m | 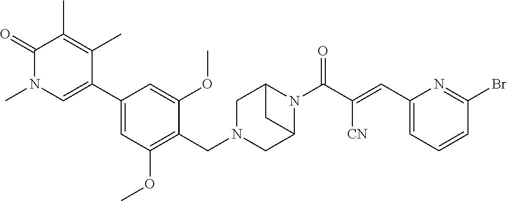 [SMILES: COc1cc(-c2cn(C)c(=O)c(C)c2C)cc(OC)c1CN1CC2CC(C1)N2C(=O)/C(C#N)=C/c1cccc(Br)n1] | 619.2/2% |
| | (E/Z)-3-(6-bromopyridin-2-yl)-2-(3-(2,6-dimethoxy-4-(1,4,5- | |
| | trimethyl-6-oxo-1,6-dihydropyridin-3-yl)benzyl)-3,6- | |
| | diazabicyclo[3.1.1]heptane-6-carbonyl)acrylonitrile | |
| BRD9n | 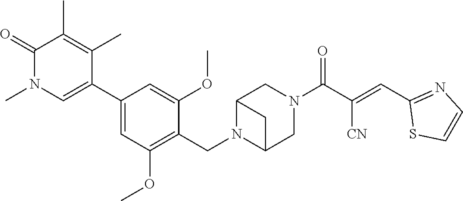 [SMILES: COc1cc(-c2cn(C)c(=O)c(C)c2C)cc(OC)c1CN1C2CC1CN(C(=O)/C(C#N)=C/c1nccs1)C2] | 546.2/13% |
| | (E/Z)-2-(6-(2,6-dimethoxy-4-(1,4,5-trimethyl-6-oxo-1,6- | |
| | dihydropyridin-3-yl)benzyl)-3,6-diazabicyclo[3.1.1]heptane- | |
| | 3-carbonyl)-3-(thiazol-2-yl)acrylonitrile | |
| BRD9o | 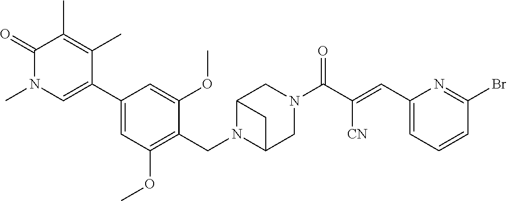 [SMILES: COc1cc(-c2cn(C)c(=O)c(C)c2C)cc(OC)c1CN1C2CC1CN(C(=O)/C(C#N)=C/c1cccc(Br)n1)C2] | 619.2/6% |
| | (E/Z)-3-(6-bromopyridin-2-yl)-2-(6-(2,6-dimethoxy-4-(1,4,5- | |
| | trimethyl-6-oxo-1,6-dihydropyridin-3-yl)benzyl)-3,6- | |
| | diazabicyclo[3.1.1]heptane-3-carbonyl)acrylonitrile | |
| BRD9p | 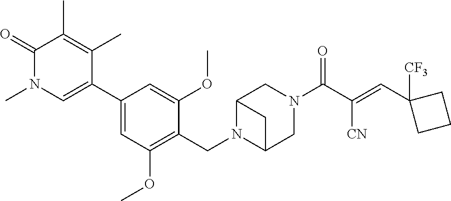 [SMILES: COc1cc(-c2cn(C)c(=O)c(C)c2C)cc(OC)c1CN1C2CC1CN(C(=O)/C(C#N)=C/C1(C(F)(F)F)CCC1)C2] | 585.6/9% |
| | (E/Z)-2-(6-(2,6-dimethoxy-4-(1,4,5-trimethyl-6-oxo-1,6- | |
| | dihydropyridin-3-yl)benzyl)-3,6-diazabicyclo[3.1.1]heptane- | |
| | 3-carbonyl)-3-(1-(trifluoromethyl)cyclobutyl)acrylonitrile | |
| BRD9y | 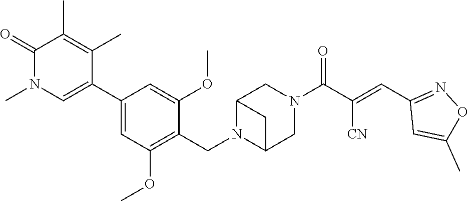 [SMILES: COc1cc(-c2cn(C)c(=O)c(C)c2C)cc(OC)c1CN1C2CC1CN(C(=O)/C(C#N)=C/c1cc(C)on1)C2] | 544.6/23% |
| | (E/Z)-2-(6-(2,6-dimethoxy-4-(1,4,5-trimethyl-6-oxo-1,6- | |
| | dihydropyridin-3-yl)benzyl)-3,6-diazabicyclo[3.1.1]heptane- | |
| | 3-carbonyl)-3-(5-methylisoxazol-3-yl)acrylonitrile | |
| BRD9q | 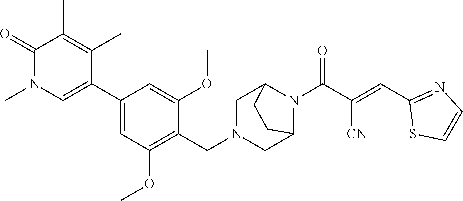 [SMILES: COc1cc(-c2cn(C)c(=O)c(C)c2C)cc(OC)c1CN1CC2CCC(C1)N2C(=O)/C(C#N)=C/c1nccs1] | 560.2/75% |
| | (E/Z)-2-(3-(2,6-dimethoxy-4-(1,4,5-trimethyl-6-oxo-1,6- | |
| | dihydropyridin-3-yl)benzyl)-3,8-diazabicyclo[3.2.1]octane-8- | |
| | carbonyl)-3-(thiazol-2-yl)acrylonitrile | |
| BRD9z | 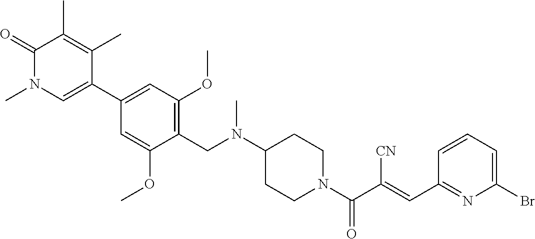 [SMILES: COc1cc(-c2cn(C)c(=O)c(C)c2C)cc(OC)c1CN(C)C1CCN(C(=O)/C(C#N)=C/c2cccc(Br)n2)CC1] | 634.2/4% |
| | (E/Z)-3-(6-bromopyridin-2-yl)-2-(4-((2,6-dimethoxy-4-(1,4,5- | |
| | trimethyl-6-oxo-1,6-dihydropyridin-3- | |
| | yl)benzyl)(methyl)amino)piperidine-1-carbonyl)acrylonitrile | |
| BRD9aa | 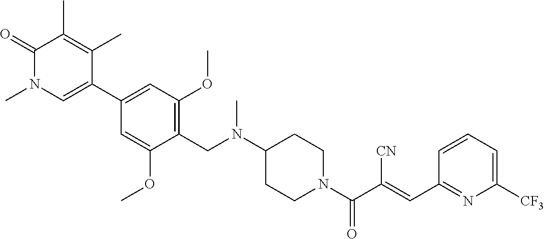 [SMILES: COc1cc(-c2cn(C)c(=O)c(C)c2C)cc(OC)c1CN(C)C1CCN(C(=O)/C(C#N)=C/c2cccc(C(F)(F)F)n2)CC1] | 624.7/10% |
| | (E/Z)-2-(4-((2,6-dimethoxy-4-(1,4,5-trimethyl-6-oxo-1,6- | |
| | dihydropyridin-3-yl)benzyl)(methyl)amino)piperidine-1- | |
| | carbonyl)-3-(6-(trifluoromethyl)pyridin-2-yl)acrylonitrile | |
| BRD9r | 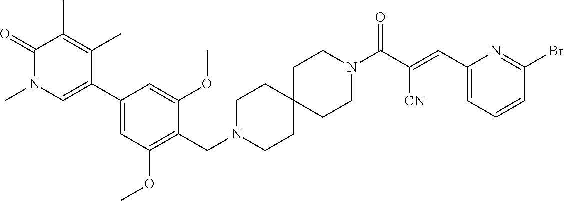 [SMILES: COc1cc(-c2cn(C)c(=O)c(C)c2C)cc(OC)c1CN1CCC2(CC1)CCN(C(=O)/C(C#N)=C/c1cccc(Br)n1)CC2] | 675.6/11% |
| | (E/Z)-3-(6-bromopyridin-2-yl)-2-(9-(2,6-dimethoxy-4-(1,4,5- | |
| | trimethyl-6-oxo-1,6-dihydropyridin-3-yl)benzyl)-3,9- | |
| | diazaspiro[5.5]undecane-3-carbonyl)acrylonitrile | |

TABLE 6a
The following examples were prepared following general procedure 7a using
starting materials synthesized in Table 4a.
Table 6a

| | | LCMS m/z |
|---|---|---|
| Name | Product Structure/Name | [M + H]⁺/Yield |
| BRD9af | 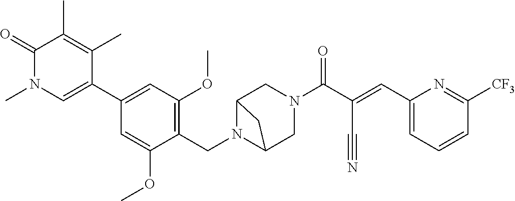 [SMILES: COc1cc(-c2cn(C)c(=O)c(C)c2C)cc(OC)c1CN1C2CC1CN(C(=O)/C(C#N)=C/c1cccc(C(F)(F)F)n1)C2] | 608.2/17% |
| | (E)-2-(6-(2,6-dimethoxy-4-(1,4,5-trimethyl-6-oxo-1,6- | |
| | dihydropyridin-3-yl)benzyl)-3,6-diazabicyclo[3.1.1]heptane- | |
| | 3-carbonyl)-3-(6-(trifluoromethyl)pyridin-2-yl)acrylonitrile | |

TABLE 7a
The following examples were prepared following general procedure 8a using
starting materials synthesized in Table 4a.
Table 7a

| | | LCMS m/z |
|---|---|---|
| Name | Product Structure/Name | [M + H]⁺/Yield |
| BRD9ag | 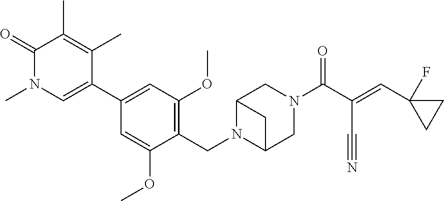 [SMILES: COc1cc(-c2cn(C)c(=O)c(C)c2C)cc(OC)c1CN1C2CC1CN(C(=O)/C(C#N)=C/C1(F)CC1)C2] | 521.2/25% |
| | (E)-2-(6-(2,6-dimethoxy-4-(1,4,5-trimethyl-6-oxo-1,6- | |
| | dihydropyridin-3-yl)benzyl)-3,6-diazabicyclo[3.1.1]heptane- | |
| | 3-carbonyl)-3-(1-fluorocyclopropyl)acrylonitrile | |

TABLE 8a
The following examples were prepared following general procedure 6a using
starting materials synthesized in Table 4a.
Table 8a

| | | LCMS m/z |
|---|---|---|
| Name | Product Structure/Name | [M + H]⁺/Yield |
| BRD9ae | 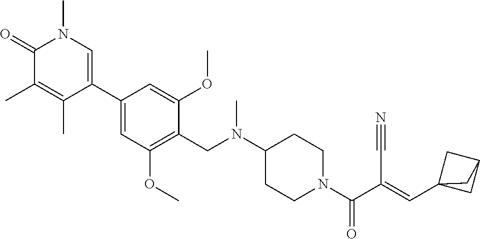 [SMILES: COc1cc(-c2cn(C)c(=O)c(C)c2C)cc(OC)c1CN(C)C1CCN(C(=O)/C(C#N)=C/C23CC(C2)C3)CC1] | 545.3/22% |
| | (E/Z)-3-(bicyclo[1.1.1]pentan-1-yl)-2-(4-((2,6-dimethoxy-4- | |
| | (1,4,5-trimethyl-6-oxo-1,6-dihydropyridin-3- | |
| | yl)benzyl)(methyl)amino)piperidine-1-carbonyl)acrylonitrile | |

TABLE 9a
The following examples were prepared following general procedure 3a using
carboxylic acid synthesized according to WO 2021/178920 (compound B11-1, page 316) and
amine specified in Table 9a.

| | | LCMS m/z |
|---|---|---|
| Amine (Y) | Product Structure/Name | [M + H]⁺/Yield |
| 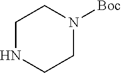 [SMILES: CC(C)(C)OC(=O)N1CCNCC1] | 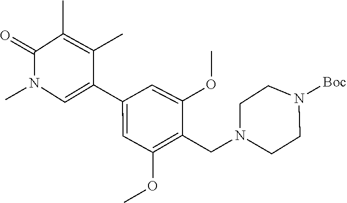 [SMILES: COc1cc(-c2cn(C)c(=O)c(C)c2C)cc(OC)c1CN1CCN(C(=O)OC(C)(C)C)CC1] | 486.4/52% |
| | tert-butyl 4-(2,6-dimethoxy-4-(1,4,5-trimethyl- | |
| | 6-oxo-1,6-dihydropyridin-3- | |
| | yl)benzoyl)piperazine-1-carboxylate | |
| 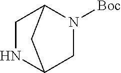 [SMILES: CC(C)(C)OC(=O)N1CC2CC1CN2] | 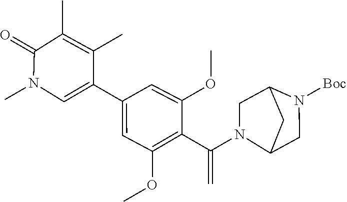 [SMILES: C=C(c1c(OC)cc(-c2cn(C)c(=O)c(C)c2C)cc1OC)N1CC2CC1CN2C(=O)OC(C)(C)C] | 498.2/43% |
| | tert-butyl 5-(2,6-dimethoxy-4-(1,4,5-trimethyl- | |
| | 6-oxo-1,6-dihydropyridin-3-yl)benzoyl)-2,5- | |
| | diazabicyclo[2.2.1]heptane-2-carboxylate | |
| 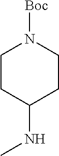 | 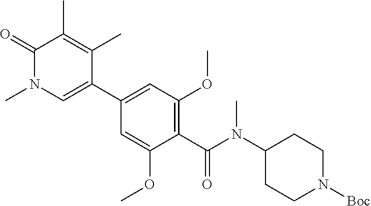 [SMILES: COc1cc(-c2cn(C)c(=O)c(C)c2C)cc(OC)c1C(=O)N(C)C1CCN(C(=O)OC(C)(C)C)CC1] | 514.2/26% |
| | tert-butyl 4-((2,6-dimethoxy-4-(1,4,5-trimethyl- | |
| | 6-oxo-1,6-dihydropyridin-3- | |
| | yl)benzyl)(methyl)amino)piperidine-1- | |
| | carboxylate | |
| 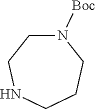 [SMILES: CC(C)(C)OC(=O)N1CCCNCC1] | 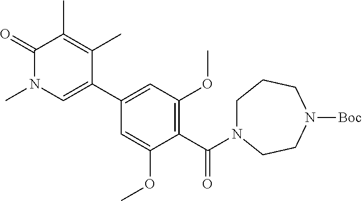 [SMILES: COc1cc(-c2cn(C)c(=O)c(C)c2C)cc(OC)c1C(=O)N1CCCN(C(=O)OC(C)(C)C)CC1] | 500.4/36% |
| | tert-butyl 4-(2,6-dimethoxy-4-(1,4,5-trimethyl- | |
| | 6-oxo-1,6-dihydropyridin-3-yl)benzoyl)-1,4- | |
| | diazepane-1-carboxylate | |

TABLE 10a
The following examples were prepared following general procedure 2a using
starting materials synthesized in Table 9a.
Table 10a

| | | LCMS m/z |
|---|---|---|
| Name | Product Structure | [M + H]⁺/Yield |
| 5-(3,5-dimethoxy-4- (piperazine-1- carbonyl)phenyl)-1,3,4- trimethylpyridin-2(1H)-one | 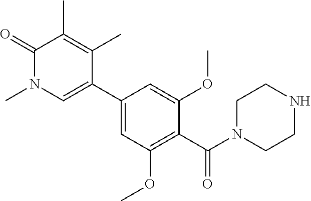 [SMILES: COc1cc(-c2cn(C)c(=O)c(C)c2C)cc(OC)c1C(=O)N1CCNCC1] | 386.5/87% |
| 5-(4-(2,5- diazabicyclo[2.2.1]heptane- 2-carbonyl)-3,5- dimethoxyphenyl)-1,3,4- trimethylpyridin-2(1H)-one | 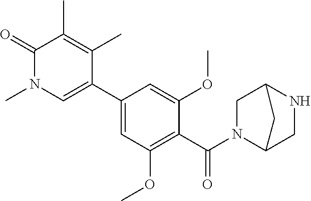 [SMILES: COc1cc(-c2cn(C)c(=O)c(C)c2C)cc(OC)c1C(=O)N1CC2CC1CN2] | 398.4/85% |
| 2,6-dimethoxy-N-methyl-N- (piperidin-4-yl)-4-(1,4,5- trimethyl-6-oxo-1,6- dihydropyridin-3- yl)benzamide | 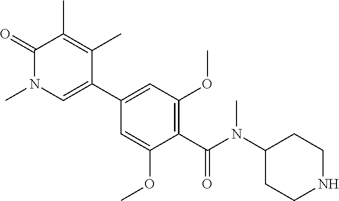 [SMILES: COc1cc(-c2cn(C)c(=O)c(C)c2C)cc(OC)c1C(=O)N(C)C1CCNCC1] | 414.2/83% |
| 5-(4-(1,4-diazepane-1- carbonyl)-3,5- dimethoxyphenyl)-1,3,4- trimethylpyridin-2(1H)-one | 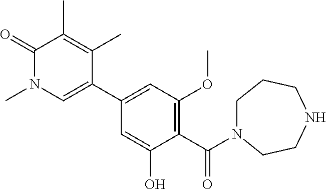 [SMILES: COc1cc(-c2cn(C)c(=O)c(C)c2C)cc(O)c1C(=O)N1CCCNCC1] | 400.6/92% |

### Synthesis of (E/Z)-2-cyano-4,4-dimethyl-pent-2-enoic acid

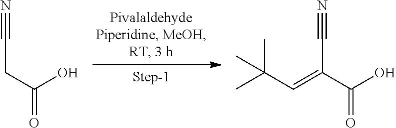

To a stirred solution of 2-cyanoacetic acid (6 g, 69.76 mmol) in methanol (60 mL) was added piperidine (6.51 g, 76.6 mmol) and pivaldehyde (11.86 g, 139 mmol) at room temperature. The resulting reaction mixture was stirred at room temperature for 3 h. The reaction was monitored by TLC; after completion, the reaction mixture was concentrated under reduced pressure and diluted with water (100 mL), then extracted with DCM. The aqueous layer was then acidified with 2 M HCl to pH 2 and extracted with DCM. Organic layer was dried over sodium sulfate, filtered and concentrated under reduced pressure to afford (E/Z)-2-cyano-4,4-dimethyl-pent-2-enoic acid (1.5g, crude) as off white solid.

¹H NMR (400 MHz, CDCl₃) δ=7.72 (s, 1H), 1.34 (s, 9H)

LC-MS: 154.3 [M+H]⁺; 94.11% at RT=1.21 min Method Details: Formic Acid (AMC-LCMS-15) Column: X Select CSH C18 2.5 μm; 3.0×50 mm Mobile Phase A: 0.05% FA in Water+5% ACN Mobile Phase B: 0.05% FA in ACN Flow Rate: 1.2 mL Oven Temperature: 50° C. Gradient Program (Time/B %): 0/2, 1.5/98,2.2/98,2.5/2,3/2.

TABLE 11a
The following examples were prepared following general procedure 3a using
starting materials synthesized in Table 10a and (E/Z)-2-cyano-4,4-dimethylpent-2-enoic acid.
Table 11a

| | | LCMS m/z |
|---|---|---|
| Name | Product Structure | [M + H]⁺/Yield |
| BRD9s | 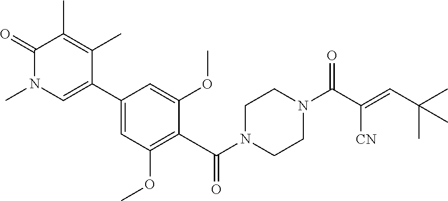 [SMILES: COc1cc(-c2cn(C)c(=O)c(C)c2C)cc(OC)c1C(=O)N1CCN(C(=O)/C(C#N)=C/C(C)(C)C)CC1] | 521.2/25% |
| | (E/Z)-2-(4-(2,6-dimethoxy-4-(1,4,5-trimethyl- | |
| | 6-oxo-1,6-dihydropyridin-3- | |
| | yl)benzoyl)piperazine-1-carbonyl)-4,4- | |
| | dimethylpent-2-enenitrile | |
| BRD9t | 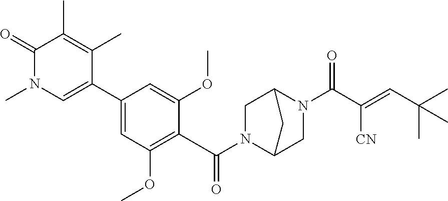 [SMILES: COc1cc(-c2cn(C)c(=O)c(C)c2C)cc(OC)c1C(=O)N1CC2CC1CN2C(=O)/C(C#N)=C/C(C)(C)C] | 533.4/12% |
| | (E/Z)-2-(5-(2,6-dimethoxy-4-(1,4,5-trimethyl- | |
| | 6-oxo-1,6-dihydropyridin-3-yl)benzoyl)-2,5- | |
| | diazabicyclo[2.2.1]heptane-2-carbonyl)-4,4- | |
| | dimethylpent-2-enenitrile | |
| BRD9u | 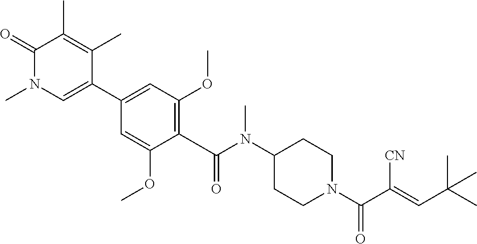 [SMILES: COc1cc(-c2cn(C)c(=O)c(C)c2C)cc(OC)c1C(=O)N(C)C1CCN(C(=O)/C(C#N)=C/C(C)(C)C)CC1] | 549.2/9% |
| | (E/Z)-2-(4-((2,6-dimethoxy-4-(1,4,5-trimethyl- | |
| | 6-oxo-1,6-dihydropyridin-3- | |
| | yl)benzyl)(methyl)amino)piperidine-1- | |
| | carbonyl)-4,4-dimethylpent-2-enenitrile | |
| BRD9v | 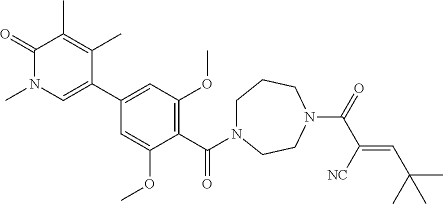 [SMILES: COc1cc(-c2cn(C)c(=O)c(C)c2C)cc(OC)c1C(=O)N1CCCN(C(=O)/C(C#N)=C/C(C)(C)C)CC1] | 535.4/18% |
| | (E/Z)-2-(4-(2,6-dimethoxy-4-(1,4,5-trimethyl- | |
| | 6-oxo-1,6-dihydropyridin-3-yl)benzoyl)-1,4- | |
| | diazepane-1-carbonyl)-4,4-dimethylpent-2- | |
| | enenitrile | |

### BRD9w: 7-bromo-5-methylthieno[3,2-c]pyridin-4(5H)-one

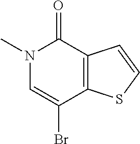

To a stirred solution of 7-bromothieno[3,2-c]pyridin-4 (5H)-one (5 g, 21.7 mmol) in DMF (50 mL) was added K₂CO₃ (6.01 g, 43.5 mmol) at 0° C. The reaction mixture was stirred for 1 h at RT, then Mel (1.49 ml, 23.9 mmol) was added and the reaction was stirred for 16 h. The reaction mixture was quenched with ice-cold water and extracted with EtOAc. The combined organic layers were dried over anhydrous sodium sulphate and concentrated in vacuo. The crude product was purified silica gel column chromatography (gradient=10% MeOH in DCM). The appropriate fractions were concentrated in vacuo to afford 7-bromo-5-methylthieno[3,2-c]pyridin-4 (5H)-one (4.5 g, 18.25 mmol, 84% yield). LCMS m/z [M+H]⁺=246.2

### 2,6-dimethoxy-4-(5-methyl-4-oxo-4,5-dihydrothieno[3,2-c]pyridin-7-yl)benzaldehyde

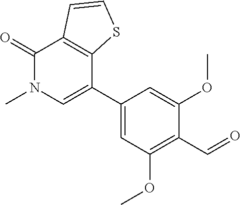 [SMILES: COc1cc(-c2cn(C)c(=O)c3ccsc23)cc(OC)c1C=O]

A stirred solution of 7-bromo-5-methylthieno[3,2-c]pyridin-4 (5H)-one (4.9 g, 20.1 mmol), 2,6-dimethoxy-4-(4,4,5,5-tetramethyl-1,3,2-dioxaborolan-2-yl)benzaldehyde (8.21 g, 28.1 mmol) and tripotassium phosphate (12.8 g, 60.2 mmol) in THF (120 mL) and water (20 mL) at RT was degassed with N₂ for 10 min. XPhos Pd G2 (0.79 g, 1.0 mmol]) was added to the reaction mixture which was heated at 80° C. for 5 h. The reaction mixture was then filtered through celite and washed with excess EtOAc. The filtrate was concentrated in vacuo and the crude compound was triturated with MTBE before being dried in vacuo to afford 2,6-dimethoxy-4-(5-methyl-4-oxo-4,5-dihydrothieno[3,2-c]pyridin-7-yl)benzaldehyde (6.0 g, 16.0 mmol, 88% yield). LCMS m/z [M+H]⁺=330.1

### tert-butyl 4-(2,6-dimethoxy-4-(5-methyl-4-oxo-4,5-dihydrothieno[3,2-c]pyridin-7-yl)benzyl)piperazine-1-carboxylate

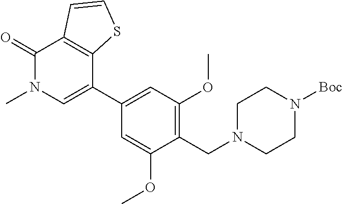 [SMILES: COc1cc(-c2cn(C)c(=O)c3ccsc23)cc(OC)c1CN1CCN(C(=O)OC(C)(C)C)CC1]

The compound was prepared following general procedure 1 using the aldehyde prepared above and tert-butyl piperazine-1-carboxylate. LCMS m/z [M+H]⁺=500.1

### 7-(3,5-dimethoxy-4-(piperazin-1-ylmethyl)phenyl)-5-methylthieno[3,2-c]pyridin-4 (5H)-one

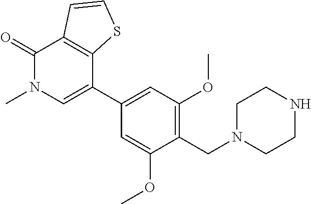 [SMILES: COc1cc(-c2cn(C)c(=O)c3ccsc23)cc(OC)c1CN1CCNCC1]

The compound was prepared following general procedure 2 using the Boc-protected amine prepared above. LCMS m/z [M+H]⁺=400.2

### (E/Z)-2-(4-(2,6-dimethoxy-4-(5-methyl-4-oxo-4,5-dihydrothieno[3,2-c]pyridin-7-yl)benzyl)piperazine-1-carbonyl)-4,4-dimethylpent-2-enenitrile

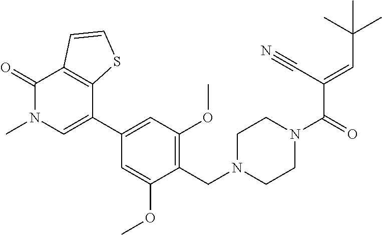 [SMILES: COc1cc(-c2cn(C)c(=O)c3ccsc23)cc(OC)c1CN1CCN(C(=O)/C(C#N)=C/C(C)(C)C)CC1]

The compound was prepared following general procedure 3 using the amine prepared above. LCMS m/z [M+H]⁺=535.2

### Additional Examples

TABLE 12a
The following exemplary compounds were prepared by General Procedure 3b using
5-(3,5-dimethoxy-4-(piperazin-1-ylmethyl)phenyl)-1,3,4-trimethylpyridin-2(1H)-one
synthesized in Table 2a and the appropriate acid selected from Pipacid 0 to 39 and
Pyracid 0 to 47 as defined above..

| | | m/z |
|---|---|---|
| Name | Structure | (M + H)⁺ |
| BRD9ba | 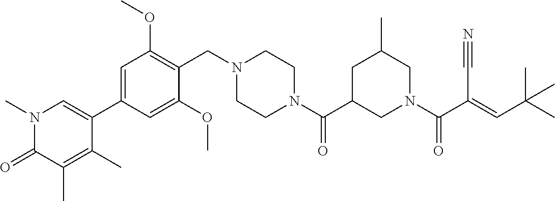 [SMILES: COc1cc(-c2cn(C)c(=O)c(C)c2C)cc(OC)c1CN1CCN(C(=O)C2CC(C)CN(C(=O)/C(C#N)=C/C(C)(C)C)C2)CC1] | 632.4 |
| | (E)-2-(3-(4-(2,6-dimethoxy-4-(1,4,5-trimethyl-6-oxo- | |
| | 1,6-dihydropyridin-3-yl)benzyl)piperazine-1-carbonyl)- | |
| | 5-methylpiperidine-1-carbonyl)-4,4-dimethylpent-2- | |
| | enenitrile | |
| BRD9bb | 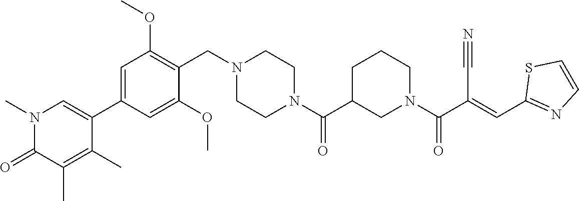 [SMILES: COc1cc(-c2cn(C)c(=O)c(C)c2C)cc(OC)c1CN1CCN(C(=O)C2CCCN(C(=O)/C(C#N)=C/c3nccs3)C2)CC1] | 645.3 |
| | (E)-2-(3-(4-(2,6-dimethoxy-4-(1,4,5-trimethyl-6-oxo-1,6-dihydropyridin-3- | |
| | yl)benzyl)piperazine-1-carbonyl)piperidine-1-carbonyl)-3-(thiazol-2- | |
| | yl)acrylonitrile | |
| BRD9bc | 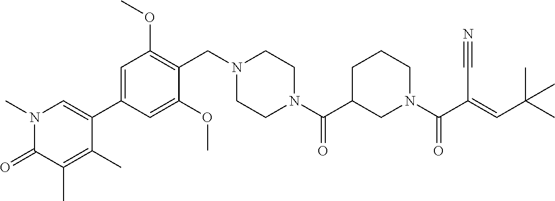 [SMILES: COc1cc(-c2cn(C)c(=O)c(C)c2C)cc(OC)c1CN1CCN(C(=O)C2CCCN(C(=O)/C(C#N)=C/C(C)(C)C)C2)CC1] | 618.4 |
| | (E)-2-(3-(4-(2,6-dimethoxy-4-(1,4,5-trimethyl-6-oxo-1,6-dihydropyridin-3 | |
| | yl)benzyl)piperazine-1-carbonyl)piperidine-1-carbonyl)-4,4-dimethylpent- | |
| | enenitrile | |
| BRD9bd | 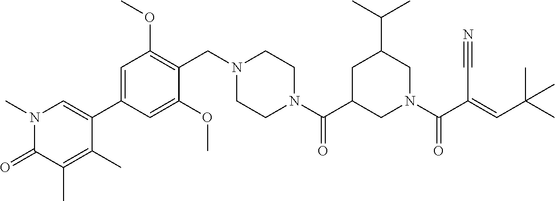 [SMILES: COc1cc(-c2cn(C)c(=O)c(C)c2C)cc(OC)c1CN1CCN(C(=O)C2CC(C(C)C)CN(C(=O)/C(C#N)=C/C(C)(C)C)C2)CC1] | 660.4 |
| | (E)-2-(3-(4-(2,6-dimethoxy-4-(1,4,5-trimethyl-6-oxo-1,6-dihydropyridin-3- | |
| | yl)benzyl)piperazine-1-carbonyl)-5-isopropylpiperidine-1-carbonyl)-4,4- | |
| | dimethylpent-2-enenitrile | |
| BRD9be | 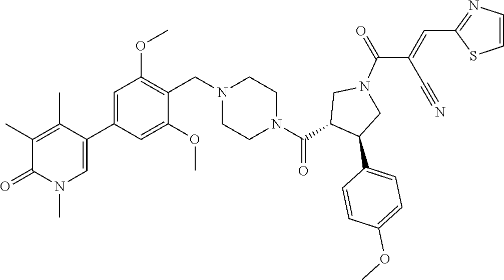 [SMILES: COc1ccc([C@@H]2CN(C(=O)/C(C#N)=C/c3nccs3)C[C@H]2C(=O)N2CCN(Cc3c(OC)cc(-c4cn(C)c(=O)c(C)c4C)cc3OC)CC2)cc1] | 737.3 |
| | (E)-2-((3S,4R)-3-(4-(2,6-dimethoxy-4-(1,4,5-trimethyl-6-oxo-1,6-dihydrop | |
| | yl)benzyl)piperazine-1-carbonyl)-4-(4-methoxyphenyl)pyrrolidine-1-carb | |
| | (thiazol-2-yl)acrylonitrile | |
| BRD9bf | 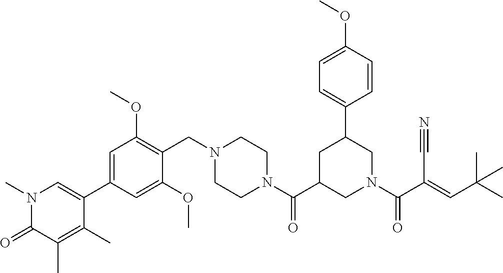 [SMILES: COc1ccc(C2CC(C(=O)N3CCN(Cc4c(OC)cc(-c5cn(C)c(=O)c(C)c5C)cc4OC)CC3)CN(C(=O)/C(C#N)=C/C(C)(C)C)C2)cc1] | 724.4 |
| | (E)-2-(3-(4-(2,6-dimethoxy-4-(1,4,5-trimethyl-6-oxo-1,6-dihydropyridin-3- | |
| | yl)benzyl)piperazine-1-carbonyl)-5-(4-methoxyphenyl)piperidine-1- | |
| | carbonyl)-4,4-dimethylpent-2-enenitrile | |
| BRD9bg | 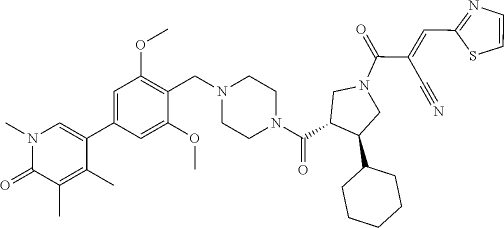 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2C2CCCCC2)CC1] | 713.4 |
| | (E)-2-((3S,4S)-3-cyclohexyl-4-(4-(2,6-dimethoxy-4-(1,4,5-trimethyl-6- | |
| | oxo-1,6-dihydropyridin-3-yl)benzyl)piperazine-1-carbonyl)pyrrolidine- | |
| | 1-carbonyl)-3-(thiazol-2-yl)acrylonitrile | |
| BRD9bh | 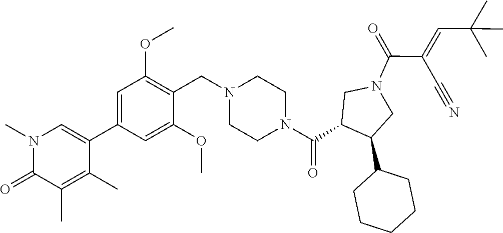 [SMILES: COc1cc(-c2cn(C)c(=O)c(C)c2C)cc(OC)c1CN1CCN(C(=O)[C@@H]2CN(C(=O)/C(C#N)=C/C(C)(C)C)C[C@H]2C2CCCCC2)CC1] | 686.4 |
| | (E)-2-((3S,4S)-3-cyclohexyl-4-(4-(2,6-dimethoxy-4-(1,4,5-trimethyl-6- | |
| | oxo-1,6-dihydropyridin-3-yl)benzyl)piperazine-1-carbonyl)pyrrolidine- | |
| | 1-carbonyl)-4,4-dimethylpent-2-enenitrile | |
| BRD9bi | 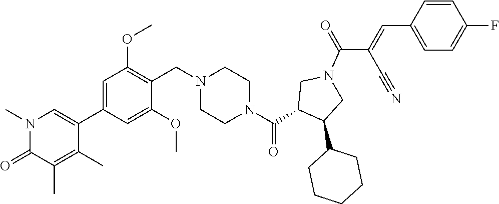 [SMILES: COc1cc(-c2cn(C)c(=O)c(C)c2C)cc(OC)c1CN1CCN(C(=O)[C@@H]2CN(C(=O)/C(C#N)=C/c3ccc(F)cc3)C[C@H]2C2CCCCC2)CC1] | 724.4 |
| | (E)-2-((3S,4S)-3-cyclohexyl-4-(4-(2,6-dimethoxy-4-(1,4,5- | |
| | trimethyl-6-oxo-1,6-dihydropyridin-3-yl)benzyl)piperazine- | |
| | 1-carbonyl)pyrrolidine-1-carbonyl)-3-(4- | |
| | fluorophenyl)acrylonitrile | |
| BRD9bj | 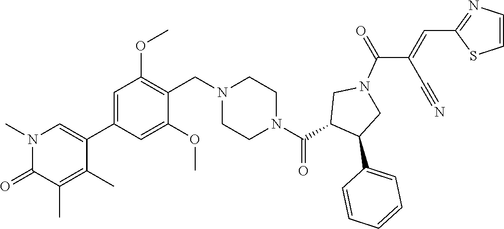 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2c2ccccc2)CC1] | 707.3 |
| | (E)-2-((3S,4R)-3-(4-(2,6-dimethoxy-4-(1,4,5-trimethyl-6- | |
| | oxo-1,6-dihydropyridin-3-yl)benzyl)piperazine-1-carbonyl)- | |
| | 4-phenylpyrrolidine-1-carbonyl)-3-(thiazol-2-yl)acrylonitrile | |
| BRD9bk | 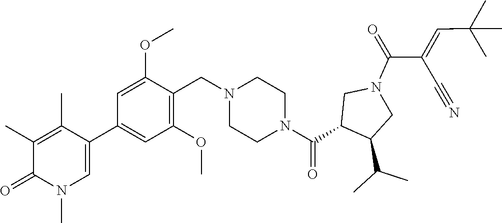 [SMILES: COc1cc(-c2cn(C)c(=O)c(C)c2C)cc(OC)c1CN1CCN(C(=O)[C@@H]2CN(C(=O)/C(C#N)=C/C(C)(C)C)C[C@H]2C(C)C)CC1] | 646.4 |
| | (E)-2-((3S,4S)-3-(4-(2,6-dimethoxy-4-(1,4,5-trimethyl-6-oxo-1,6- | |
| | dihydropyridin-3-yl)benzyl)piperazine-1-carbonyl)-4- | |
| | isopropylpyrrolidine-1-carbonyl)-4,4-dimethylpent-2-enenitrile | |
| BRD9bl | 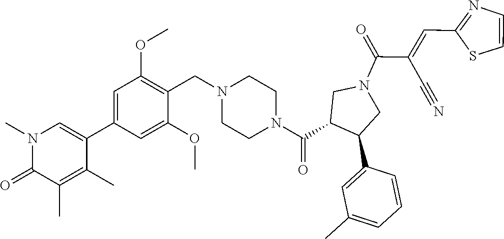 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2c2cccc(C)c2)CC1] | 721.3 |
| | (E)-2-((3S,4R)-3-(4-(2,6-dimethoxy-4-(1,4,5-trimethyl-6-oxo- | |
| | 1,6-dihydropyridin-3-yl)benzyl)piperazine-1-carbonyl)-4-(m- | |
| | tolyl)pyrrolidine-1-carbonyl)-3-(thiazol-2-yl)acrylonitrile | |
| BRD9bm | 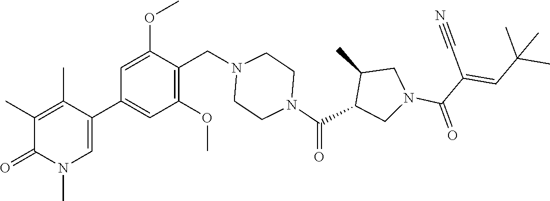 [SMILES: COc1cc(-c2cn(C)c(=O)c(C)c2C)cc(OC)c1CN1CCN(C(=O)[C@H]2CN(C(=O)/C(C#N)=C/C(C)(C)C)C[C@@H]2C)CC1] | 618.4 |
| | (E)-2-((3R,4R)-3-(4-(2,6-dimethoxy-4-(1,4,5-trimethyl-6-oxo-1,6- | |
| | dihydropyridin-3-yl)benzyl)piperazine-1-carbonyl)-4-methylpyrrolidine-1- | |
| | carbonyl)-4,4-dimethylpent-2-enenitrile | |

### SMARCA2/SMARCA4 Degraders

Protein degrading compounds that have an E3 ligase binding portion and a SMARCA2 or SMARCA4 binding portion wherein the SMARCA2 or SMARCA4 binding ligand binds to SMARCA2 or SMARCA4 and brings it to the ligase for ultimate degradation by the proteasome are described in WO 2021/155321 A², WO 2021/207291 A¹, WO 2021/142247 A¹, WO 2021/133920 A¹, WO 2020/251972 A¹, WO 2020/251971 A¹, WO 2020/251969 A¹, WO 2020/010227 A¹, WO 2021/083949 A¹, WO 2021/086785 A¹, WO 2021/067606 A¹, WO 2019/207538 A¹, WO 2020/078933 A¹, US20200038378 A¹, and WO 2019/195201 A¹.

### LCMS

Method-A: Column: X-Select CSH C18 (3.0*50 mm, 2.5 μm), Mobile Phase A: 0.05% FA in H₂O Mobile Phase B: 0.05% FA in ACN, Gradient % B: 0/2,0.3/2,2.0/98,2.8/98,3.0/2,3.7/2 Flow Rate: 1.0 ml/min

Method-B: Column: Bakerbond Q2100 C18 1.8 μm; 2.1×50 mm Mobile Phase A: 0.05% FA in Water Mobile Phase B: 0.05% FA in ACN Flow Rate: 0.6 ml Gradient Program (Time/B %): 0/5,0.2/5,2.3/98,3.3/98,3.8/5,4.5/5

Method-C: Column: X Select CSH C18 2.5 μm; 3.0×50 mm Mobile Phase A: 2.5 mM Ammonium Bicarbonate Water+5% ACN Mobile Phase B: ACN Flow Rate: 1.2 ml Gradient Program (Time/B %): 0/0, 1.5/100,2.4/100,2.6/0,3/0

Method-D: Column: X-Bridge BEH C18 (3.0*50 mm, 2.5 μm), Mobile Phase A: 0.05% FA in H2O: CAN (95:5), Mobile % B: Phase B: 0.05% FA in ACN, Gradient 0/2,0.2/2,2.2/98,3/98,3.2/2,4/2 Flow Rate: 1.2 ml/min

### Preparative Purification Method

Method: Column: Synergy (150*20 mm);5 μm Mobile Phase A: 0.1% FA in Water Mobile Phase B: 100% ACN Flow Rate: 18 ml/min Gradient: linear gradient.

### Amide Coupling—General Procedure 1b

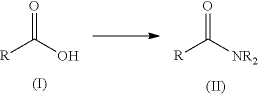

To a stirred solution of carboxylic acid (I) (1.5 equiv.) in DMF was added DIPEA (2.5 equiv.) and HATU (1.5 equiv.). The reaction mixture was stirred for 5 min, then relevant amine (1.0 equiv.) was added and the reaction mixture was stirred for 16 h at RT. The reaction was quenched with ice cold water and extracted with EtOAc. The combined organic layers were concentrated in vacuo to afford the crude product. Where stated, the crude product was purified by silica gel column chromatography/reverse phase preparative HPLC to give the desired amide (II).

### Synthesis of (E/Z)-2-cyano-4,4-dimethyl-pent-2-enoic acid

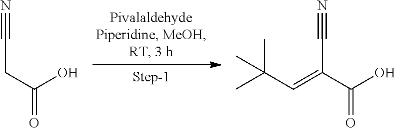

To a stirred solution of 2-cyanoacetic acid (6 g, 69.76 mmol) in methanol (60 mL) was added piperidine (6.51 g, 76.6 mmol) and pivaldehyde (11.86 g, 139 mmol) at room temperature. The resulting reaction mixture was stirred at room temperature for 3 h. The reaction was monitored by TLC; after completion, the reaction mixture was concentrated under reduced pressure and diluted with water (100 mL), then extracted with DCM. The aqueous layer was then acidified with 2 M HCl to pH 2 and extracted with DCM. Organic layer was dried over sodium sulfate, filtered and concentrated under reduced pressure to afford (E/Z)-2-cyano-4,4-dimethyl-pent-2-enoic acid (1.5g, crude) as off white solid.

¹H NMR (400 MHz, CDCl₃) δ=7.72 (s, 1H), 1.34 (s, 9H)

LC-MS: 154.3 [M+H]⁺; 94.11% at RT=1.21 min Method Details: Formic Acid (AMC-LCMS-15) Column: X Select CSH C18 2.5 μm; 3.0×50 mm Mobile Phase A: 0.05% FA in Water+5% ACN Mobile Phase B: 0.05% FA in ACN Flow Rate: 1.2 mL Oven Temperature: 50° C. Gradient Program (Time/B %): 0/2, 1.5/98,2.2/98,2.5/2,3/2.

### Synthesis of 2-(6-amino-5-(3,8-diazabicyclo[3.2.1]octan-3-yl)pyridazin-3-yl)-4-fluorophenol

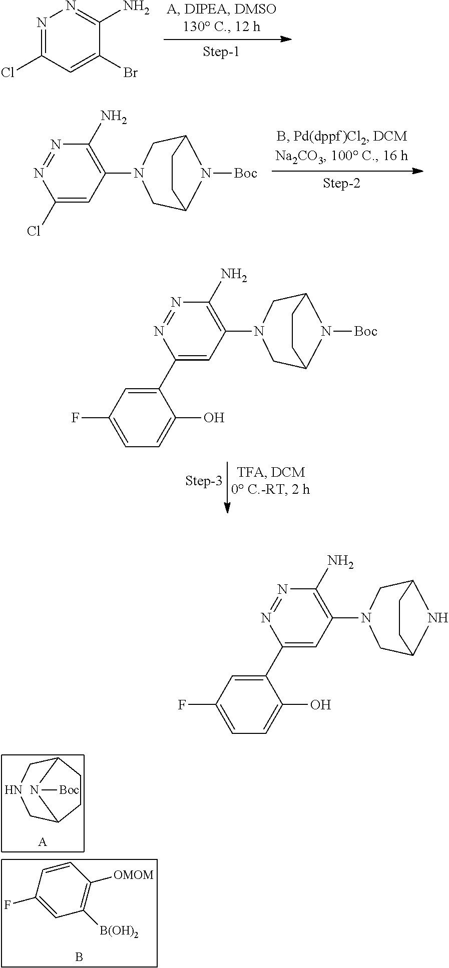

### Synthesis of tert-butyl 3-(3-amino-6-chloropyridazin-4-yl)-3,8-diazabicyclo[3.2.1]octane-8-carboxylate

To a stirring solution of 4-bromo-6-chloropyridazin-3-amine (4 g, 19.23 mmol) in DMSO (40 mL) was added tert-butyl 3,8-diazabicyclo[3.2.1]octane-8-carboxylate (compound A; 6 g, 28.7 mmol) followed by DIPEA (9.92 mL, 76.76 mmol) at room temperature. The reaction mixture was heated to 130° C. and stirred for 12 h. The reaction was monitored by TLC and LCMS. After completion, the reaction mixture was extracted with ethyl acetate (250 mL×2) and water (100 mL×2). Combined organic layer was dried over sodium sulfate and concentrated under reduced pressure to afford tert-butyl 3-(3-amino-6-chloropyridazin-4-yl)-3,8-diazabicyclo[3.2.1]octane-8-carboxylate (4 g, crude) as an off white solid.

¹H NMR (400 MHz, DMSO-d₆) δ=9.05 (br s, 1H), 7.00 (s, 2H), 5.99 (br s, 2H), 4.13 (br s, 2H), 3.44 (d, J=11.3 Hz, 4H), 3.14-3.06 (m, 2H), 2.99 (d, J=12.6 Hz, 2H), 2.26-2.18 (m, 2H), 1.98-1.87 (m, 2H), 1.18 (t, J=7.3 Hz, 2H)

LC-MS: 339.9 (M+H)+; 99.17% at RT: 2.004 min. Column: X select CSH-C18 (3.0λ⁵⁰ mm, 2.5 μm) Mobile Phase: A: 0.05% FA in water, Mobile Phase B: 0.05% FA in ACN T/B %: 0/2,0.3/2,2.0/98,3.0/98,3.2/2,4.0/2 Flow rate: 1.0 ml/min (Gradient)

### Synthesis of tert-butyl 3-(3-amino-6-chloropyridazin-4-yl)-3,8-diazabicyclo[3.2.1]octane-8-carboxylate

To a stirring solution of tert-butyl 3-(3-amino-6-chloropyridazin-4-yl)-3,8-diazabicyclo[3.2.1]octane-8-carboxylate′ (0.5 g, 1.47 mmol) in 1,4-dioxane and water (1 mL, 3:1) was added (5-fluoro-2-(methoxymethoxy)phenyl) boronic acid (compound B; 0.35 g, 1.76 mmol) followed by Na₂CO₃ (0.31 g, 2.94 mmol) at room temperature. The reaction mixture was degassed with argon for 20 min, and then Pd(dppf)Cl₂·DCM (0.12 g, 0.15 mmol) was added at the same temperature. The reaction mixture was heated to 100° C. and stirred for 16 h. The reaction was monitored by TLC. After completion, the reaction mixture was extracted with ethyl acetate (250 mL×2) and diluted with water (100 mL×2). The combined organic layer was dried over sodium sulfate and concentrated under reduced pressure. The crude product was purified by medium pressure liquid chromatography, eluted with 80% ethyl acetate in heptane to afford tert-butyl 3-(3-amino-6-chloropyridazin-4-yl)-3,8-diazabicyclo[3.2.1]octane-8-carboxylate (0.22 g, 31%) as pale yellow solid.

¹H NMR (400 MHz, DMSO-d₆) δ=13.97 (br s, 1H), 7.96-7.88 (m, 2H), 7.58 (s, 2H), 7.13-7.03 (m, 2H), 6.88 (dd, J=5.0, 8.6 Hz, 1H), 6.04 (br s, 1H), 4.24 (br s, 2H), 2.92 (d, J=12.7 Hz, 2H), 2.10-1.98 (m, 2H), 1.86 (br s, 2H), 1.43 (s, 9H)

LC-MS: 416.8 (M+H)⁺; 98.36% at RT: 2.25 min. Column: Bakerbond Q2100 C18 1.8 μm; 2.1×50 mm Mobile Phase A: 0.05% FA in Water B: 0.05% FA in ACN Flow Rate: 0.6 ml/min Gradient: Time/% B: 0/5_0.2/5_2.3/98_3.3/98_3.8/5_4.5/5

### Synthesis of 2-(6-amino-5-(3,8-diazabicyclo[3.2.1]octan-3-yl)pyridazin-3-yl)-4-fluorophenol

To a stirring solution of Int-2 (0.22 g, 0.53 mmol) in DCM (2 mL) was added TFA (1 mL) at 0° C. The reaction mixture was allowed to warm to room temperature and stirred for 2 h. The reaction was monitored by TLC. After completion, the reaction mixture was concentrated under reduced pressure. The crude product was purified by medium pressure liquid chromatography, eluted with 5% methanol in DCM to afford 2-(6-amino-5-(3,8-diazabicyclo[3.2.1]octan-3-yl)pyridazin-3-yl)-4-fluorophenol (0.12 g, 70%) as an off white solid. ¹H NMR (400 MHz, CD3OD) δ=7.57 (dd, J=3.0, 10.1 Hz, 1H), 7.50 (s, 1H), 7.04-6.98 (m, 1H), 6.90 (dd, J=5.0, 9.0 Hz, 1H), 3.70 (br s, 2H), 3.42-3.35 (m, 2H), 2.97 (d, J=11.5 Hz, 2H), 2.16-2.07 (m, 2H), 2.01-1.92 (m, 2H), exchangeable protons are not observed in 1H NMR.

LC-MS: 316.4 (M+H)⁺; 99.85% at RT: 1.35 min. Column: X-Select CSH C18, (50 mm*3.0 mm, 2.5u) Mobile Phase A: 0.05% TFA in Water Mobile Phase B: 0.05% TFA in Acetonitrile Flow rate: 1.0 mL/min. Gradient Program (B %): 0.0/2, 0.3/2,2.0/98, 3/98, 3.2/2,4.0/2

HPLC: 99.07%, RT: 5.692 min. Column: X-Bridge C18 (4.6*150) mm 5u Mobile Phase: A-5 mM Ammonium Bicarbonate in water B-Acetonitrile Flow Rate: 1.0. mL/minute Gradient program: Time (min)/B Conc: 0.01/5, 1.0/5, 8.0/100, 12.0/100, 14.0/5, 18.0/5

### Synthesis of (R)-2-(6-amino-5-(3-aminopiperidin-1-yl)pyridazin-3-yl)phenol

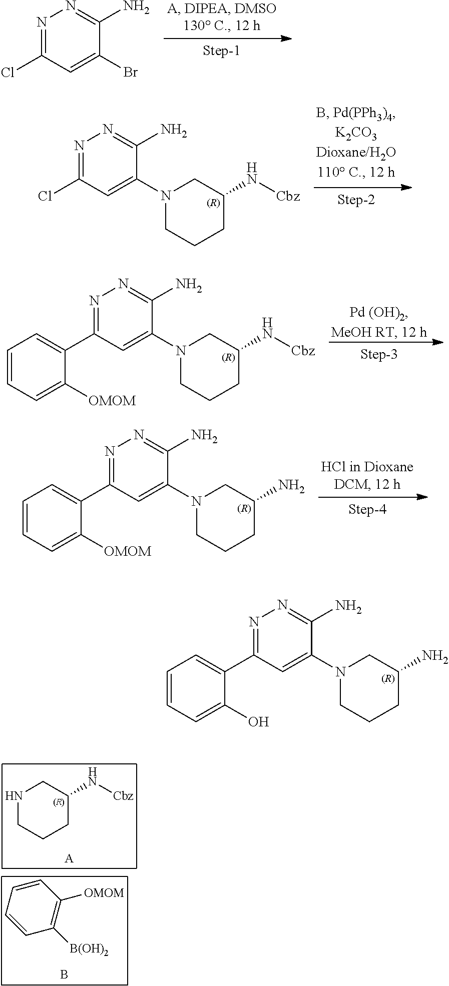

### Synthesis of benzyl (R)-(1-(3-amino-6-chloropyridazin-4-yl) piperidin-3-yl)carbamate

To a stirring solution of 4-bromo-6-chloropyridazin-3-amine (2.0 g, 9.6 mmol) in DMSO (15 mL) was added benzyl (R)-piperidin-3-ylcarbamate (compound A; 2.69 g, 11.5 mmol) followed by DIPEA (7.08 mL, 38.4 mmol) at room temperature. The reaction mixture was heated to 130° C. and stirred for 12 h. The reaction was monitored by TLC. After completion, the reaction mixture was quenched with ice cold water, and solid was precipitated. The precipitate was filtered, and the filtrate was dried under vacuum to afford benzyl (R)-(1-(3-amino-6-chloropyridazin-4-yl) piperidin-3-yl) carbamate (1.1 g, 31%) as brown solid.

LC-MS: 362.4 (M+H)+; 96.09% at RT: 1.686 min. Column: >select CSH-C18 (3.0×⁵⁰ mm, 2.5 μm) Mobile Phase: A: 0.5 ml Formic acid in950 ml water+50 ml ACN, B: 0.5 ml Formic acid in ACN T/B %: 0.01/2,0.2/2,2.2/98,3/98,3.2/2,4/2 Flow rate: 1.2 ml/min (Gradient)

### Synthesis of benzyl (R)-(1-(3-amino-6-(2-(methoxymethoxy)phenyl)pyridazin-4-yl)piperidin-3-yl)carbamate

To a stirring solution of benzyl (R)-(1-(3-amino-6-chloropyridazin-4-yl) piperidin-3-yl) carbamate (1.0 g, 2.7 mmol) in a mixture of 1,4-dioxane and water (15 mL) was added K₂CO₃ (1.14 g, 8.3 mmol), (2-(methoxymethoxy)phenyl) boronic acid (compound B; 0.75 g, 4.1 mmol) at room temperature and the reaction mixture was degassed for 10 min. Then to the reaction mixture was added Pd(PPh₃)₄ (0.31 g, 0.27 mmol) at the same temperature. The resulting reaction mixture was heated to 110° C. and stirred for 12 h. The reaction was monitored by TLC. After completion, the reaction mixture was concentrated under reduced pressure. The crude product was purified by medium pressure liquid chromatography, eluted with 5% methanol in DCM to afford benzyl (R)-(1-(3-amino-6-(2-(methoxymethoxy)phenyl)pyridazin-4-yl)piperidin-3-yl)carbamate (0.42 g, 33%) as yellow solid.

LC-MS: 464.6 (M+H)+; 97.12% at RT: 1.027 min. Column: X Select CSH C18 2.5 μm; 3.0×50 mm Mobile Phase A: 0.05% FA in Water+5% ACN Mobile Phase B: 0.05% FA in ACN Flow Rate: 1.2 ml Oven Temperature: 50° C. Gradient Program (Time/B %): 0/2, 1.5/98,2.2/98,2.5/2,3/2

### Synthesis of (R)-4-(3-aminopiperidin-1-yl)-6-(2-(methoxymethoxy)phenyl)pyridazin-3-amine

To a degassed solution of benzyl (R)-(1-(3-amino-6-(2-(methoxymethoxy)phenyl)pyridazin-4-yl)piperidin-3-yl)carbamate (1.0 g, 2.7 mmol) in methanol (40 mL) was added Pd(OH)₂ (0.5 g) at room temperature and the reaction mixture was stirred for 12 h under hydrogen gas pressure. The reaction was monitored by TLC. After completion, the reaction mixture was filtered through a celite bed, and the filtrate was concentrated under reduced pressure to afford (R)-4-(3-aminopiperidin-1-yl)-6-(2-(methoxymethoxy)phenyl)pyridazin-3-amine (1.0 g, 70%) as off white solid. The crude product was used as such in next step without further purification.

¹H NMR (400 MHz, DMSO-d₆) δ=7.63 (dd, J=1.6, 7.5 Hz, 1H), 7.39-7.31 (m, 2H), 7.22-7.14 (m, 2H), 7.12-7.04 (m, 1H), 5.96 (d, J=14.4 Hz, 2H), 5.21 (s, 2H), 3.20-3.14 (m, 2H), 3.01 (dd, J=3.6, 7.3 Hz, 4H), 2.78-2.64 (m, 1H), 2.31 (s, 1H), 1.89-1.74 (m, 4H), 1.64 (d, J=3.1, 8.8 Hz, 1H), 1.37-1.24 (m, 1H)

### Synthesis of (R)-2-(6-amino-5-(3-aminopiperidin-1-yl)pyridazin-3-yl)phenol

To a stirring solution of (R)-4-(3-aminopiperidin-1-yl)-6-(2-(methoxymethoxy)phenyl)pyridazin-3-amine (0.1 g, 0.30 mmol) in DCM (3 mL) was added 4 M HCl (1 mL, in dioxane) at room temperature and stirred for 12 h. The reaction was monitored by TLC. After completion, the reaction mixture was concentrated under reduced pressure. The crude product was washed with ether and concentrated under reduced pressure to afford (R)-2-(6-amino-5-(3-aminopiperidin-1-yl)pyridazin-3-yl)phenol (0.06 g, 70%) as pale yellow solid.

¹H NMR (400 MHz, CD₃OD) δ=7.77 (d, J=1.8 Hz, 1H), 7.75 (d, J=1.4 Hz, 1H), 7.49 (s, 1H), 7.26 (d, J=1.6 Hz, 1H), 7.24 (dd, J=0.9, 1.5 Hz, 1H), 7.22 (d, J=1.6 Hz, 1H), 6.93-6.89 (m, 1H), 3.42 (dd, J=3.4, 11.5 Hz, 1H), 3.26-3.19 (m, 1H), 3.12-3.05 (m, 1H), 2.92-2.84 (m, 1H), 2.65 (dd, J=8.5, 11.3 Hz, 1H), 2.03-1.89 (m, 1H), 1.82-1.72 (m, 1H)

LC-MS: 286.0 (M+H)+; 99.56% at RT: 1.89 min. Column: X-Select CSH C18, (50 mm*3.0 mm, 2.5μ) Mobile Phase A: 2.5 mM Ammonium Bicarbonate in Water+5% ACN Mobile Phase B: 100% ACN Flow rate: 1.0 mL/min. Column temperature: 40° C. Gradient Program (B %): 0.0/2, 0.3/2, 2.0/98, 2.8/98, 3.0/2,3.7/2

HPLC: 95.82%, RT: 4.427 min. Column: X-Select CSH C18 (4.6*150) mm 5u Mobile Phase: A-0.1% TFA in water B-Acetonitrile Flow Rate: 1.2. mL/minute Gradient program: Time (min)/B Conc: 0.01/5, 1.0/5, 8.0/100, 12.0/100, 14.0/5, 18.0/5

### Synthesis of 2-(4-(1-(4-(3-amino-6-(2-hydroxyphenyl)pyridazin-4-yl)-1H-pyrazol-1-yl)ethyl)piperidin-1-yl)acetic acid

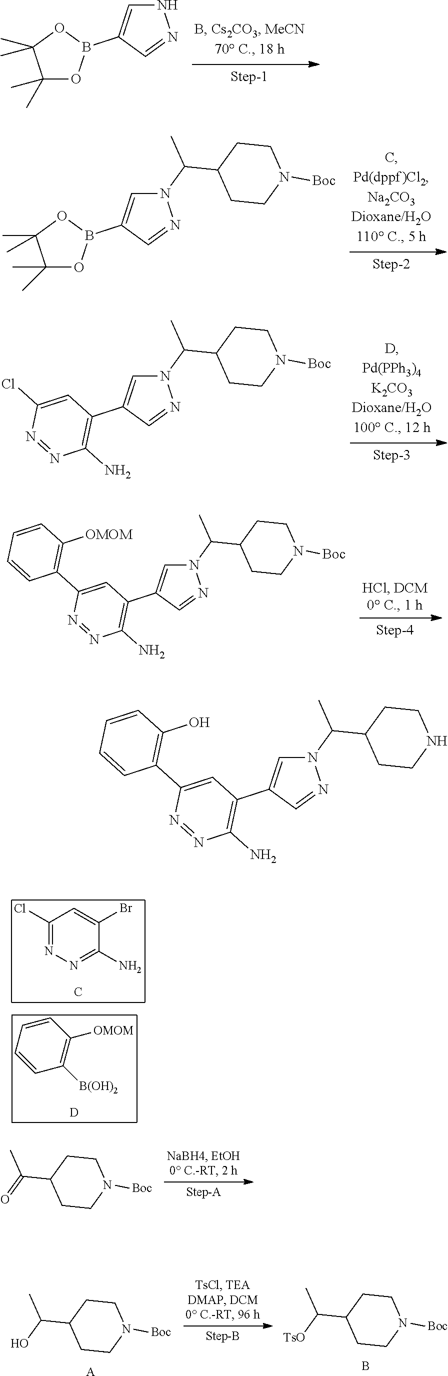

### Synthesis of tert-butyl 4-(1-hydroxyethyl)piperidine-1-carboxylate (compound A)

To a stirring solution of tert-butyl 4-acetylpiperidine-1-carboxylate (20 g, 72.20 mmol) in ethanol (200 mL) was added NaBH₄ (5.46 g, 144 mmol) at 0° C. The reaction mixture was allowed to warm to room temperature and stirred for 2 h. The reaction was monitored by TLC. After completion, the reaction mixture was quenched with ammonium chloride solution then extracted with ethyl acetate (200 mL×2) and diluted with water (100 mL). Combined organic layer was dried over sodium sulfatesulfate and concentrated under reduced pressure to afford tert-butyl 4-(1-hydroxyethyl) piperidine-1-carboxylate (20 g, quantitative yield) as brown liquid. LC-MS: 406.2 (M+H)+; 99.16% at RT: 1.59 min. Column: X-Bridge BEH C18 (3.0*50 mm, 2.5 um) Mobile Phase A: 2.5 mM Ammonium Bicarbonate Water+5% ACN Mobile Phase B: ACN Flow Rate: 1.0 ml/min Gradient Program (B %): 0.0/2, 0.3/2, 2.0/98, 3/98, 3.2/2,4.0/2

### Synthesis of tert-butyl 4-(1-(tosyloxy)ethyl)piperidine-1-carboxylate (compound B)

To a stirring solution of tert-butyl 4-(1-hydroxyethyl) piperidine-1-carboxylate (10 g, 35.84 mmol) in DCM (100 mL) was added TEA (25 mL, 179.4 mmol), PTS-CI (17 g, 89.60 mmol) followed by DMAP (0.87 g, 7.17 mmol) at 0° C. The reaction mixture was allowed to warm to room temperature and stirred for 96 h. The reaction was monitored by TLC. After completion, the reaction mixture was quenched with sodium bicarbonate solution then extracted with DCM (100 mL×2). Combined organic layer was washed with brine solution dried over sodium sulfate and concentrated under reduced pressure. The crude product was purified by medium pressure liquid chromatography, eluted with 50% ethyl acetate in hexane to afford tert-butyl 4-(1-(tosyloxy)ethyl) piperidine-1-carboxylate (6.8 g, 61%) as yellow liquid.

LC-MS: 357.3 (M−56+H)+; 82.73% at RT: 2.16 min. Column: X-Bridge BEH-C18-50×3.0 mm 2.5u Mobile Phase A: 2.5 mM Ammonium Bicarbonate in water+50mlACN Mobile phase B: 100% ACN Flow: 1.0 ml/min Gradient B %: 0.0/2_0.3/2_2.0/98_3/98_3.2/2_4.0/2

### Synthesis of tert-butyl 4-(1-(4-(4,4,5,5-tetramethyl-1,3,2-dioxaborolan-2-yl)-1H-pyrazol-1-yl)ethyl)piperidine-1-carboxylate

To a stirring solution of 4-(4,4,5,5-tetramethyl-1,3,2-dioxaborolan-2-yl)-1H-pyrazole (3.3 g, 17.01 mmol) in ACN (70 mL) was added tert-butyl 4-(1-(tosyloxy)ethyl) piperidine-1-carboxylate (compound B; 6.5 g, 17.01 mmol) followed by Cs₂CO₃ (11.06 g, 34.03 mmol) at room temperature. The reaction mixture was heated to 70° C. and stirred for 18 h. The reaction was monitored by TLC. After completion, the reaction mixture was extracted with ethyl acetate (250 mL×2) and water (100 mL×2). Combined organic layer was dried over sodium sulfate and concentrated under reduced pressure. The crude product was purified by medium pressure liquid chromatography, eluted with 30% ethyl acetate in hexane to afford tert-butyl 4-(1-(4-(4,4,5,5-tetramethyl-1,3,2-dioxaborolan-2-yl)-1H-pyrazol-1-yl)ethyl) piperidine-1-carboxylate (2 g, 29%) as yellow gummy liquid.

¹H NMR (400 MHz, DMSO-d₆) δ=7.48-7.22 (m, 2H), 3.78-3.49 (m, 11H), 3.15-3.06 (m, 1H), 2.78-2.55 (m, 2H), 2.15-2.01 (m, 2H), 1.80-1.53 (m, 6H), 1.39-1.35 (m, 12H)

### Synthesis of tert-butyl 4-(1-(4-(3-amino-6-chloropyridazin-4-yl)-1H-pyrazol-1-yl)ethyl)piperidine-1-carboxylate

To a stirring solution of tert-butyl 4-(1-(4-(4,4,5,5-tetramethyl-1,3,2-dioxaborolan-2-yl)-1H-pyrazol-1-yl)ethyl) piperidine-1-carboxylate (2.0 g, 4.94 mmol) in a mixture of 1,4 dioxane (18 mL) and water (2 mL) was added 4-bromo-6-chloropyridazin-3-amine (compound C; 1.02 g, 4.94 mmol) followed by Na₂CO₃ (1 g, 9.87 mmol) at room temperature. The reaction mixture was degassed for 30 min, and then Pd(dppf)Cl₂·DCM (0.36 g, 0.49 mmol) was added at the same temperature. The reaction mixture was heated to 110° C. and stirred for 5 h. The reaction was monitored by TLC. After completion, the reaction mixture was quenched with 10% methanol in DCM then concentrated under reduced pressure. The crude product was purified by medium pressure liquid chromatography, eluted with 50% ethyl acetate in hexane to afford tert-butyl 4-(1-(4-(3-amino-6-chloropyridazin-4-yl)-1H-pyrazol-1-yl)ethyl) piperidine-1-carboxylate (0.45 g, 22%) as brown solid.

LC-MS: 406.9 (M+H)+; 80.45% at RT: 2.09 min. Column: X select CSH-C18 (3.0×⁵⁰ mm, 2.5 μm) Mobile Phase: A: 0.05% FA in water, Mobile Phase B: 0.05% FA in ACN T/B %: 0/2,0.3/2,2.0/98,3.0/98,3.2/2,4.0/2 Flow rate: 1.0 ml/min (Gradient)

### Synthesis of tert-butyl 4-(1-(4-(3-amino-6-(2-(methoxymethoxy)phenyl)pyridazin-4-yl)-1H-pyrazol-1-yl)ethyl)piperidine-1-carboxylate

To a stirring solution of tert-butyl 4-(1-(4-(3-amino-6-chloropyridazin-4-yl)-1H-pyrazol-1-yl)ethyl) piperidine-1-carboxylate (0.45 g, 1.10 mmol) in a mixture of 1,4-dioxane (8 mL) and water (2 mL) was added (2-(methoxymethoxy)phenyl) boronic acid (compound D; 0.30 g, 1.66 mmol) followed by K₂CO₃ (0.30 g, 2.21 mmol) at room temperature. The reaction mixture was degassed for 30 min, and then tetrakis (0.25 g, 0.22 mmol) was added at the same temperature. The reaction mixture was heated to 100° C. and stirred for 12 h. The reaction was monitored by TLC. After completion, the reaction mixture was quenched with 10% methanol in DCM then concentrated under reduced pressure. The crude product was purified by medium pressure liquid chromatography, eluted with 50% ethyl acetate in hexane to afford tert-butyl 4-(1-(4-(3-amino-6-(2-(methoxymethoxy)phenyl)pyridazin-4-yl)-1H-pyrazol-1-yl)ethyl) piperidine-1-carboxylate (0.24 g, 55%) as yellow solid.

LC-MS: 340.3 (M−Boc+H)+; 33.36% at RT: 1.89 min. Column: X-Select CSH-C18-50×3.0 mm Instrument ID:-AMC-LCMS-20 Mobile Phase A: 0.05% FA in water B: 0.05% FA in ACN Flow Rate: 1.0 ml/min Gradient: Time/% B: 0.0/2_0.30/2_2.0/98_3.0/98_3.20/2_4.0/2

### Synthesis of 2-(6-amino-5-(1-(1-(piperidin-4-yl)ethyl)-1H-pyrazol-4-yl)pyridazin-3-yl)phenol

To a stirring solution of tert-butyl 4-(1-(4-(3-amino-6-(2-(methoxymethoxy)phenyl)pyridazin-4-yl)-1H-pyrazol-1-yl)ethyl) piperidine-1-carboxylate (0.25 g, 0.49 mmol) in DCM (3 mL) was added 4 M HCl (4 mL, 1.96 mmol, in 1,4-dioxane) at 0° C. and stirred for 1 h. The reaction was monitored by TLC. After completion, the reaction mixture was concentrated under reduced pressure. The crude product was washed with diethyl ether to afford 2-(6-amino-5-(1-(1-(piperidin-4-yl)ethyl)-1H-pyrazol-4-yl)pyridazin-3-yl)phenol (0.2 g, 99%) as yellow solid.

¹H NMR (400 MHz, DMSO-d₆) δ=8.89-8.73 (m, 1H), 8.54 (br s, 2H), 8.33-8.11 (m, 2H), 7.76-7.60 (m, 2H), 7.41-7.31 (m, 1H), 7.12-6.83 (m, 2H), 4.38-4.20 (m, 1H), 3.35-3.12 (m, 3H), 2.93-2.64 (m, 4H), 2.16-2.01 (m, 1H), 1.93-1.82 (m, 1H), 1.52-1.26 (m, 6H) LC-MS: 365.0 (M+H)⁺; 95.32% at RT: 1.353 min. Column: X select CSH-C18 (3.0×⁵⁰ mm, 2.5 μm) Mobile Phase: A: 0.05% FA in water, Mobile Phase B: 0.05% FA in ACN T/B %: 0/2,0.3/2,2.0/98,3.0/98,3.2/2,4.0/2 Flow rate: 1.0 ml/min (Gradient)

HPLC: 81.91%, RT: 4.852 min, Column: X-Select CSH C18 (4.6*150) mm 5u Mobile Phase: A-0.1% TFA in water B-Acetonitrile Flow Rate: 1.2. mL/minute Gradient program: Time (min)/B Conc.: 0.01/5, 1.0/5, 8.0/100, 12.0/100, 14.0/5, 18.0/5

### Synthesis of 2-(6,7,8,9-tetrahydro-5H-pyrido[3′,4′: 4,5]pyrrolo[2,3-c]pyridazin-3-yl)phenol

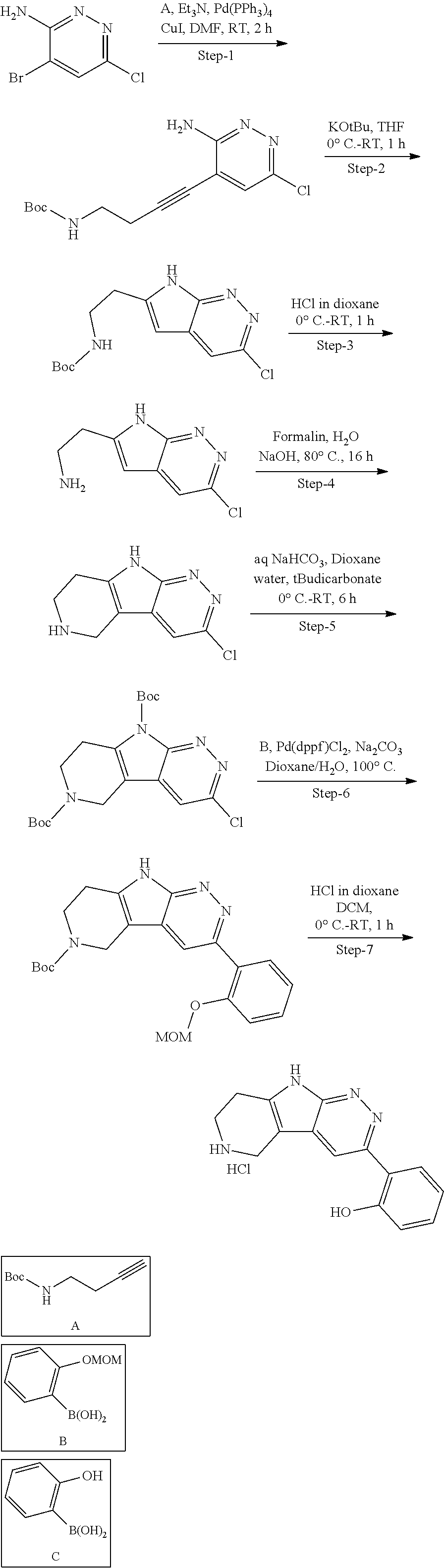

### Synthesis of tert-butyl (4-(3-amino-6-chloropyridazin-4-yl) but-3-yn-1-yl)carbamate

To a stirring solution of 4-bromo-6-chloropyridazin-3-amine SM (3 g, 14.49 mmol) in DMF (30 mL) was added tert-butyl but-3-yn-1-ylcarbamate (compound A; 2.95 g, 17.3 mmol) followed by TEA (14.67 g, 144.5 mmol) at room temperature and the reaction mixture was degassed for 10 min. Then to the reaction mixture was added CuI (0.27 g, 1.44 mmol) followed by Pd(PPh₃)₄ (0.84 g, 0.7 mmol) at room temperature and the reaction mixture was stirred for 16 h. The reaction was monitored by TLC. After completion, the reaction mixture was quenched with water and extracted with ethyl acetate. Combined organic layer was distilled and concentrated under reduced pressure. The crude product was purified by medium pressure liquid chromatography, eluted with 30% ethyl acetate in hexane to afford tert-butyl (4-(3-amino-6-chloropyridazin-4-yl) but-3-yn-1-yl) carbamate (3.2 g, 79%) as brown solid.

¹H NMR (400 MHz, DMSO-d₆) δ=7.67-7.58 (m, 1H), 7.56 (d, J=6.4 Hz, 1H), 7.46 (s, 2H), 7.39 (d, J=4.9 Hz, 1H), 7.12 (br s, 2H), 6.80 (br s, 4H), 3.19 (q, J=5.9 Hz, 5H), 2.61 (t, J=6.1 Hz, 5H), 1.38 (s, 9H)

LC-MS: 333.0 (M+H)+; 86.19% at RT: 2.623 min. Column: X select CSH-C18 (3.0×⁵⁰ mm, 2.5 μm) Mobile Phase: A: 0.05% FA in water, Mobile Phase B: 0.05% FA in can T/B %: 0/2,0.3/2,2.0/98,3.0/98,3.2/2,4.0/2 Flow rate: 1.0 ml/min (Gradient)

### Synthesis of tert-butyl(2-(3-chloro-7H-pyrrolo[2,3328yridazinezin-6-yl)ethyl)carbamate

To a stirring solution of tert-butyl (4-(3-amino-6-chloropyridazin-4-yl) but-3-yn-1-yl) carbamate (3 g, 10.3 mmol) in THF (10 mL) was added KOtBu (2.27 g, 20.2 mmol) at 0° C. The reaction mixture was allowed to warm to room temperature and stirred for 1 h. The reaction was monitored by TLC and LCMS. After completion, the reaction mixture was quenched with water and extracted with THF. Combined organic layer was dried over Na₂SO₄, filtered and the filtrate was distilled to give crude. The crude product was washed with heptane (15 mL) to afford tert-butyl(2-(3-chloro-7H-pyrrolo[2,3-c]pyridazin-6-yl)ethyl) carbamate (2.8 g, 93%) as brown solid.

¹H NMR (400 MHz, DMSO-d₆) δ=7.91-7.72 (m, 1H), 7.68-7.49 (m, 1H), 7.42-7.35 (m, 1H), 7.31-7.18 (m, 1H), 7.04-6.89 (m, 1H), 6.37 (br s, 1H), 2.93 (br s, 2H), 1.55 (br s, 9H) LC-MS: 296.9 (M+H)⁺; 73.81% at RT: 1.927 min. Column: >select CSH-C18 (3.0×⁵⁰ mm, 2.5 μm) Mobile Phase: A: 0.05% FA in water, Mobile Phase B: 0.05% FA in ACN T/B %: 0/2,0.3/2,2.0/98,3.0/98,3.2/2,4.0/2 Flow rate: 1.0 ml/min (Gradient)

### Synthesis of 2-(3-chloro-7H-pyrrolo[2,3-c]pyridazin-6-yl) ethan-1-amine

To a stirring solution of tert-butyl(2-(3-chloro-7H-pyrrolo[2,3-c]pyridazin-6-yl)ethyl) carbamate (2.8 g, 9.46 mmol) in DCM (15 mL) was added 4 M HCl (15 mL, in dioxane) at 0° C. The reaction mixture was allowed to warm to room temperature and stirred for 1 h. The reaction was monitored by TLC. After completion, the reaction mixture was concentrated under reduced pressure to afford 2-(3-chloro-7H-pyrrolo[2,3-c]pyridazin-6-yl) ethan-1-amine (1.69 g, 91%) as brown solid.

¹H NMR (400 MHz, DMSO-d₆) δ=8.23 (br s, 1H), 7.90 (s, 2H), 6.43 (s, 1H), 5.15 (br s, 2H), 3.31-3.16 (m, 2H)

LC-MS: 197.09 (M+H)⁺; 80.08% at RT: 0.21 min. Column: CORTECS UPLC C18 (3×30 mm, 1.6 μm) Mobile Phase: A: 0.05% FA in Water, B: 0.05% FA in ACN T/B %: 0/3,0.1/3, 1.4/97,2/97,2.05/3,2.5/3. Flow rate: 0.85 ml/min (Gradient)

### Synthesis of 3-chloro-6,7,8,9-tetrahydro-5H-pyrido[3′,4′:4,5]pyrrolo[2,3-c]pyridazine

To a stirring solution of 2-(3-chloro-7H-pyrrolo[2,3-c]pyridazin-6-yl) ethan-1-amine (0.5 g, 2.54 mmol) in water (10 mL) was added formalin (381 mg, 12.71 mmol) followed by sodium hydroxide (508 mg, 12.71 mmol) at 0° C. The reaction mixture was heated to 80° C. and stirred for 16 h. The reaction was monitored by LCMS. After completion, the reaction mixture was evaporated under reduced pressure to afford 3-chloro-6,7,8,9-tetrahydro-5H-pyrido[3′,4′:4,5]pyrrolo[2,3-c]pyridazine (0.5 g, 94.24%) as black solid.

LC-MS: 209.16 (M+H)+; 85.53% at RT: 0.21 min. Column: CORTECS UPLC C18 (3×30 mm, 1.6 μm) Mobile Phase: A: 0.05% FA in Water B: 0.05% FA in ACN T/B %: 0/3,0.1/3, 1.4/97,2/97,2.05/3,2.5/3. Flow rate: 0.85 ml/min (Gradient)

### Synthesis of di-tert-butyl 3-chloro-7,8-dihydro-5H-pyrido[3′,4′:4,5]pyrrolo[2,3-c]pyridazine-6,9-dicarboxylate

To a stirring solution of 3-chloro-6,7,8,9-tetrahydro-5H-pyrido[3′,4′:4,5]pyrrolo[2,3-c]pyridazine (0.5 g, 2.39 mmol) in a mixture of 1,4-dioxane and water (5 mL, 1:1) was added sodium bicarbonate (602 mg, 7.19 mmol) at 0° C., followed by di-tert-butyl dicarbonate (781 mg, 3.59 mmol). The reaction mixture was allowed to warm to room temperature and stirred for 6 h. The reaction was monitored by TLC. After completion, the reaction mixture was quenched with water and extracted with ethyl acetate. Combined organic layer was concentrated under reduced pressure. The crude product was purified by medium pressure liquid chromatography, eluted with 20% EtoAc in heptane to afford di-tert-butyl 3-chloro-7,8-dihydro-5H-pyrido[3′,4′:4,5]pyrrolo[2,3-c]pyridazine-6,9-dicarboxylate (0.3 g, 30.62%) as an off white solid.

LC-MS: 409.25 (M+H)+; 95.25% at RT: 1.68 min. Column: CORTECS UPLC C18 (3×30 mm, 1.6 μm) Mobile Phase: A: 0.05% FA in Water B: 0.05% FA in ACN T/B %: 0/3,0.1/3, 1.4/97,2/97,2.05/3,2.5/3. Flow rate: 0.85 ml/min (Gradient)

### Synthesis of tert-butyl 3-(2-(methoxymethoxy)phenyl)-5,7,8,9-tetrahydro-6H-pyrido[3′,4′: 4,5]pyrrolo[2,3-c]pyridazine-6-carboxylate

To a stirring solution of di-tert-butyl 3-chloro-7,8-dihydro-5H-pyrido[3′,4′:4,5]pyrrolo[2,3-c]pyridazine-6,9-dicarboxylate (0.3 g, 0.73 mmol) in 1,4-dioxane (4 mL) and water (1 mL) was added 2-(methoxymethoxy)phenylboronic acid (199 mg, 1.101 mmol), Na₂CO₃ (1 g, 2.20 mmol). The reaction mixture was degassed for 10 min, and then Pd(dppf)Cl₂·DCM (30.2 mg, 0.037 mmol) was added. The reaction was monitored by LCMS. After completion, the reaction mixture was diluted with water and extracted with ethyl acetate (15 mL×2). Combined organic layer was dried over sodium sulfate, filtered and concentrated under reduced pressure. The crude product was purified by medium pressure liquid chromatography, eluted with 100% ethyl acetate to afford tert-butyl 3-(2-(methoxymethoxy)phenyl)-5,7,8,9-tetrahydro-6H-pyrido[3′,4′:4,5]pyrrolo[2,3-c]pyridazine-6-carboxylate (0.18 g, 59.77%) as yellow solid.

LC-MS: 411.33 (M+H)+; 93.23% at RT: 1.20 min. Column: CORTECS UPLC C18 (3×30 mm, 1.6 μm) Mobile Phase: A: 0.05% FA in Water B: 0.05% FA in ACN T/B %: 0/3,0.1/3, 1.4/97,2/97,2.05/3,2.5/3. Flow rate: 0.85 ml/min (Gradient)/min

### Synthesis of 2-(6,7,8,9-tetrahydro-5H-pyrido[3′,4′:4,5]pyrrolo[2,3-c]pyridazin-3-yl)phenol

To a stirred solution of tert-butyl 3-(2-(methoxymethoxy)phenyl)-5,7,8,9-tetrahydro-6H-pyrido[3′,4′:4,5]pyrrolo[2,3-c]pyridazine-6-carboxylate (0.18 g, 0.44 mmol) in DCM (2 mL) was added HCl (4.0 mol/L) in dioxane (4.0 mol/L) at 0° C. and the reaction mixture was stirred at room temperature for 1 h. The reaction was monitored by TLC. After completion, the reaction mixture was concentrated under reduced pressure. The crude product was triturated with diethyl ether (10 mL×2) to afford 2-(6,7,8,9-tetrahydro-5H-pyrido[3′,4′:4,5]pyrrolo[2,3-c]pyridazin-3-yl)phenol (0.10 g, 89.92%; HCl salt) as brown solid.

¹H NMR (400 MHz, DMSO-d₆) δ=8.62 (s, 1H), 7.88 (d, J=7.8 Hz, 1H), 7.36 (t, J=7.6 Hz, 1H), 7.04-6.98 (m, 2H), 4.41 (s, 2H), 3.58-3.53 (m, 3H), 3.23-3.17 (m, 2H), 2.67 (br s, 1H), 2.33 (br s, 1H)

LC-MS: 267.2 (M+H)+; 96.85% at RT: 0.791 min. Column: X-Select CSH C18 (3.0*50 mm, 2.5 μm), Mobile Phase A: 0.05% FA in water Mobile Phase B: 0.05% FA in ACN, Gradient % B: 0/2,0.3/2,2.0/98,2.8/98,3.0/2,3.7/2 Flow Rate: 1.0 ml/min

HPLC: 93.65%, RT: 6.447 min, Column: X-Bridge C18 (4.6*150) mm 5u Mobile Phase: A-5 mM Ammonium Bicarbonate in water B—Acetonitrile Inj Volume; 5.0 μL, Flow Rate: 1.0. mL/minute Gradient program: Time (min)/B Conc: 0.01/5, 1.0/5, 8.0/100, 12.0/100, 14.0/5, 18.0/5

### Synthesis of (R)-2-(6-amino-5-(3-aminopiperidin-1-yl)pyridazin-3-yl)phenol

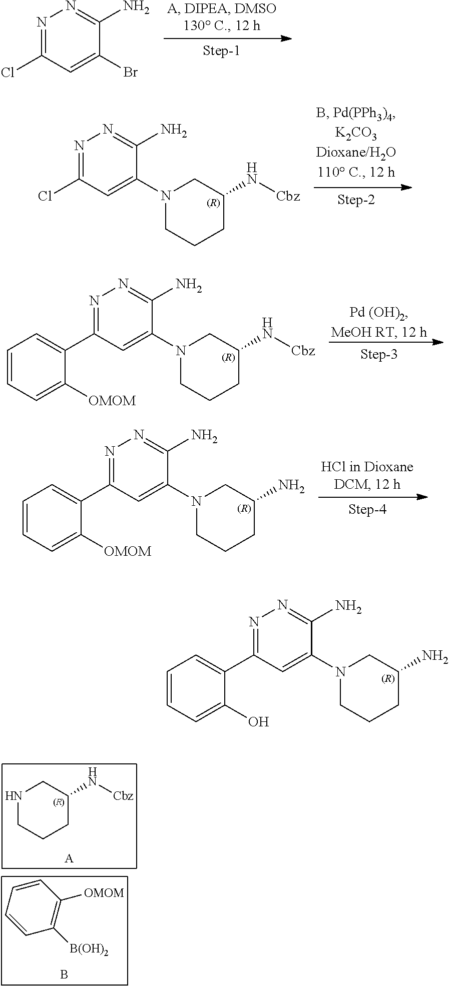

### Synthesis of benzyl (R)-(1-(3-amino-6-chloropyridazin-4-yl)piperidin-3-yl)carbamate

To a stirring solution of 4-bromo-6-chloropyridazin-3-amine (2.0 g, 9.6 mmol) in DMSO (15 mL) was added benzyl (R)-piperidin-3-ylcarbamate (compound A; 2.69 g, 11.5 mmol) followed by DIPEA (7.08 mL, 38.4 mmol) at room temperature. The reaction mixture was heated to 130° C. and stirred for 12 h. The reaction was monitored by TLC. After completion, the reaction mixture was quenched with ice cold water, and solid was precipitated. The precipitate was filtered, and the filtrate was dried under vacuum to afford benzyl (R)-(1-(3-amino-6-chloropyridazin-4-yl) piperidin-3-yl)carbamate (1.1 g, 31%) as brown solid.

LC-MS: 362.4 (M+H)+; 96.09% at RT: 1.686 min. Column: X select CSH-C18 (3.0×50 mm, 2.5 μm) Mobile Phase: A: 0.5 ml Formic acid in950 ml water+50 ml ACN, B: 0.5 ml Formic acid in ACN T/B %: 0.01/2,0.2/2,2.2/98,3/98,3.2/2,4/2 Flow rate: 1.2 ml/min (Gradient)

### Synthesis of benzyl (R)-(1-(3-amino-6-(2-(methoxymethoxy)phenyl)pyridazin-4-yl)piperidin-3-yl)carbamate

To a stirring solution of benzyl (R)-(1-(3-amino-6-chloropyridazin-4-yl)piperidin-3-yl)carbamate (1.0 g, 2.7 mmol) in a mixture of 1,4-dioxane and water (15 mL) was added K₂CO₃ (1.14 g, 8.3 mmol), (2-(methoxymethoxy)phenyl) boronic acid (compound B; 0.75 g, 4.1 mmol) at room temperature and the reaction mixture was degassed for 10 min. Then to the reaction mixture was added Pd(PPh₃)₄ (0.31 g, 0.27 mmol) at the same temperature. The resulting reaction mixture was heated to 110° C. and stirred for 12 h. The reaction was monitored by TLC. After completion, the reaction mixture was concentrated under reduced pressure. The crude product was purified by medium pressure liquid chromatography, eluted with 5% methanol in DCM to afford benzyl (R)-(1-(3-amino-6-(2-(methoxymethoxy)phenyl)pyridazin-4-yl)piperidin-3-yl)carbamate (0.42 g, 33%) as yellow solid.

LC-MS: 464.6 (M+H)+; 97.12% at RT: 1.027 min. Column: X Select CSH C18 2.5 μm; 3.0×50 mm Mobile Phase A: 0.05% FA in Water+5% ACN Mobile Phase B: 0.05% FA in ACN Flow Rate: 1.2 ml Oven Temperature: 50° C. Gradient Program (Time/B %): 0/2, 1.5/98,2.2/98,2.5/2,3/2

### Synthesis of (R)-4-(3-aminopiperidin-1-yl)-6-(2-(methoxymethoxy)phenyl)pyridazin-3-amine

To a degassed solution of benzyl (R)-(1-(3-amino-6-(2-(methoxymethoxy)phenyl)pyridazin-4-yl)piperidin-3-yl)carbamate (1.0 g, 2.7 mmol) in methanol (40 mL) was added Pd(OH)₂ (0.5 g) at room temperature and the reaction mixture was stirred for 12 h under hydrogen gas pressure. The reaction was monitored by TLC. After completion, the reaction mixture was filtered through a celite bed, and the filtrate was concentrated under reduced pressure to afford (R)-4-(3-aminopiperidin-1-yl)-6-(2-(methoxymethoxy)phenyl)pyridazin-3-amine (1.0 g, 70%) as off white solid. The crude product was used as such in next step without further purification.

¹H NMR (400 MHz, DMSO-d₆) δ=7.63 (dd, J=1.6, 7.5 Hz, 1H), 7.39-7.31 (m, 2H), 7.22-7.14 (m, 2H), 7.12-7.04 (m, 1H), 5.96 (d, J=14.4 Hz, 2H), 5.21 (s, 2H), 3.20-3.14 (m, 2H), 3.01 (dd, J=3.6, 7.3 Hz, 4H), 2.78-2.64 (m, 1H), 2.31 (s, 1H), 1.89-1.74 (m, 4H), 1.64 (d, J=3.1, 8.8 Hz, 1H), 1.37-1.24 (m, 1H)

### Synthesis of (R)-2-(6-amino-5-(3-aminopiperidin-1-yl)pyridazin-3-yl)phenol

To a stirring solution of (R)-4-(3-aminopiperidin-1-yl)-6-(2-(methoxymethoxy)phenyl)pyridazin-3-amine (0.1 g, 0.30 mmol) in DCM (3 mL) was added 4 M HCl (1 mL, in dioxane) at room temperature and stirred for 12 h. The reaction was monitored by TLC. After completion, the reaction mixture was concentrated under reduced pressure. The crude product was washed with ether and concentrated under reduced pressure to afford (R)-2-(6-amino-5-(3-aminopiperidin-1-yl)pyridazin-3-yl)phenol (0.06 g, 70%) as pale yellow solid.

¹H NMR (400 MHz, CD₃OD) δ=7.77 (d, J=1.8 Hz, 1H), 7.75 (d, J=1.4 Hz, 1H), 7.49 (s, 1H), 7.26 (d, J=1.6 Hz, 1H), 7.24 (dd, J=0.9, 1.5 Hz, 1H), 7.22 (d, J=1.6 Hz, 1H), 6.93-6.89 (m, 1H), 3.42 (dd, J=3.4, 11.5 Hz, 1H), 3.26-3.19 (m, 1H), 3.12-3.05 (m, 1H), 2.92-2.84 (m, 1H), 2.65 (dd, J=8.5, 11.3 Hz, 1H), 2.03-1.89 (m, 1H), 1.82-1.72 (m, 1H)

LC-MS: 286.0 (M+H)+; 99.56% at RT: 1.89 min. Column: X-Select CSH C18, (50 mm*3.0 mm, 2.5μ) Mobile Phase A: 2.5 mM Ammonium Bicarbonate in Water+5% ACN Mobile Phase B: 100% ACN Flow rate: 1.0 mL/min. Column temperature: 40° C. Gradient Program (B %): 0.0/2, 0.3/2, 2.0/98, 2.8/98, 3.0/2,3.7/2

HPLC: 95.82%, RT: 4.427 min. Column: X-Select CSH C18 (4.6*150) mm 5u Mobile Phase: A-0.1% TFA in water B-Acetonitrile Flow Rate: 1.2. mL/minute Gradient program: Time (min)/B Conc: 0.01/5, 1.0/5, 8.0/100, 12.0/100, 14.0/5, 18.0/5

### Synthesis of (E)-2-cyano-4,4-dimethyl-N-[2-(methylamino)ethyl]pent-2-enamide

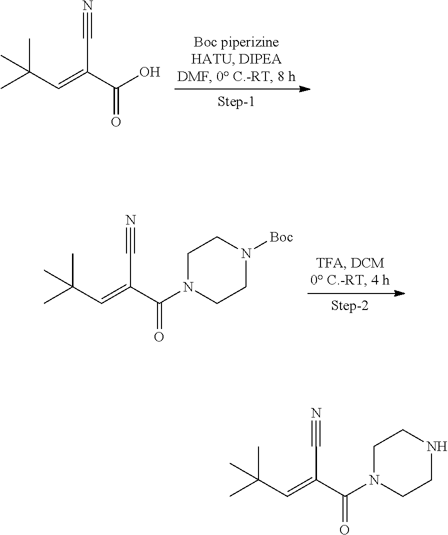 [SMILES: CC(C)(C)/C=C(\C#N)C(=O)O ; CC(C)(C)/C=C(\C#N)C(=O)N1CCN(C(=O)OC(C)(C)C)CC1 ; CC(C)(C)/C=C(\C#N)C(=O)N1CCNCC1]

### Synthesis of tert-butyl 4-[(E)-2-cyano-4,4-dimethyl-pent-2-enoyl]piperazine-1-carboxylate

To a stirred solution of (E)-2-cyano-4,4-dimethyl-pent-2-enoic acid (1 g, 6.530 mmol) and Boc-Piperazine (851 mg, 4.570 mmol) in DMF (10 ml) was added HATU (3.70 g, 9.75 mmol). Reaction mixture was cooled to 0° C. and DIPEA (2.11 ml, 16.5 mmol) was added dropwise. The resulting reaction mixture was stirred at RT for 8 h. The reaction was monitored by TLC; after completion, the reaction mixture was diluted with cold water (100 ml) and extracted the compound with ethyl acetate. The combined organic layer was dried over sodium sulphate, filtered and concentrated under reduced pressure. The crude was purified by flash chromatography to afford tert-butyl 4-[(E)-2-cyano-4,4-dimethyl-pent-2-enoyl]piperazine-1-carboxylate (800 mg, 40%) as an off white solid.

LC-MS: 222.1 [M−Boc+H]⁺; 82.06% at RT=1.36 min Method Conditions: Column: Xselect CSH-C18 (3.0×50 mm, 2.5 μm) Mobile Phase: A: 0.05% FA in Water+5% ACN, B: 0.05% FA in ACN

### Synthesis of (E)-2-cyano-4,4-dimethyl-N-[2-(methylamino)ethyl]pent-2-enamide

To a stirred solution of tert-butyl 4-[(E)-2-cyano-4,4-dimethyl-pent-2-enoyl]piperazine-1-carboxylate in DCM (4 ml) was added TFA (8 ml, 104.4 mmol) dropwise at 0° C. The resulting reaction mixture was stirred at RT for 4 h. The reaction was monitored by TLC; after completion, the reaction mixture was concentrated under reduced pressure. Semi solid mass obtained was triturated with diethyl ether and dried under reduced pressure to afford (E)-2-cyano-4,4-dimethyl-N-[2-(methylamino)ethyl]pent-2-enamide (476 mg, crude) as brown semi solid.

¹H NMR (400 MHz, DMSO-d₆) δ=3.75-3.55 (m, 4H), 3.17-3.13 (m, 4H), 2.67 (s, 1H), 1.22 (s, 9H)

LC-MS: 222.3 [M+H]⁺; 68.22% at RT=1.158 min Column: Xselect CSH-C18 (3.0×50 mm, 2.5 μm) Mobile Phase: A: 0.05% FA in water, MobilePhase: B: 0.05% FA in ACN T/B %: 0./2,0.3/2,2.0/98,3.0/98,3.2/2,3.5/2 Flow rate: 1.0 ml/min (Gradient), Column Oven Temp: 40° C.

### Synthesis of (E)-2-(4-(2-cyano-4,4-dimethylpent-2-enoyl)piperazin-1-yl)acetic acid

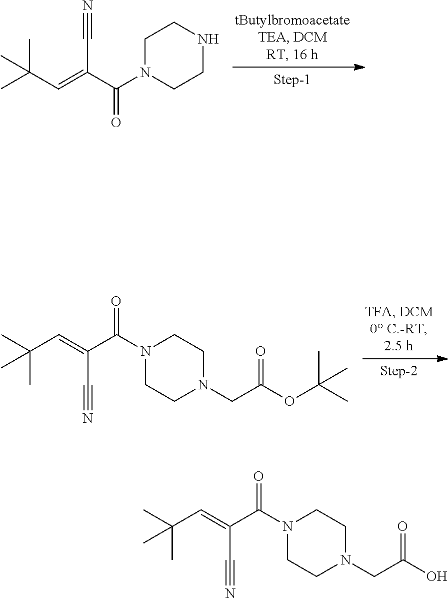

### Synthesis of tert-butyl (E)-2-(4-(2-cyano-4,4-dimethylpent-2-enoyl)piperazin-1-yl)acetate

To a stirring solution of (E)-2-cyano-4,4-dimethyl-N-[2-(methylamino)ethyl]pent-2-enamide (0.45 g, 2.03 mmol) in DCM (3 mL) was added TEA (0.61 mL, 6.09 mmol) at 10° C. followed by an addition of t-Butyl bromoacetate (0.59 g, 3.04 mmol). The reaction mixture was allowed to warm to room temperature and stirred for 16 h. The reaction was monitored by TLC; after completion, the reaction mixture was diluted with water and extracted with DCM. Combined organic layer was separated and aqueous layer was washed with DCM. Organic layer was distilled and concentrated under reduced pressure. The crude product was purified by medium pressure liquid chromatography, eluted with 70-80% ethyl acetate to afford tert-butyl (E)-2-(4-(2-cyano-4,4-dimethylpent-2-enoyl)piperazin-1-yl)acetate (0.3 g, 44%) as pale yellow solid.

¹H NMR (400 MHz, DMSO-d₆) δ=6.83-6.71 (m, 1H), 3.47 (br s, 4H), 3.22-3.10 (m, 3H), 1.55 (br s, 1H), 1.41 (br s, 9H), 1.28-1.14 (m, 9H), 1.07 (br s, 2H)

LC-MS: 74.06% 336.6 [M+H]⁺ at 1.62 min; Column: X select CSH-C18 (3.0×50 mm, 2.5 μm) Mobile Phase: A: 0.05% FA in Water+5% ACN, B: 0.05% FA in ACN T/B %: 0/2,0.3/2,2.0/98,2.8/98,3.0/2,3.5/2. Flow rate: 1.0 ml/min (Gradient)

### Synthesis of (E)-2-(4-(2-cyano-4,4-dimethylpent-2-enoyl)piperazin-1-yl)acetic acid (A³⁹)

To a stirring solution of tert-butyl (E)-2-(4-(2-cyano-4,4-dimethylpent-2-enoyl) piperazin-1-yl)acetate (0.3 g, 0.89 mmol) in DCM (3 mL) was added TFA (2.1 mL) at 0° C. The resulting reaction mixture was allowed to warm to room temperature and stirred for 2.5 h. The reaction was monitored by TLC; after completion, the reaction mixture was distilled off solvent completely. The combined organic layer was co-distilled with DCM 2-3 times then washed with diethyl ether to afford (E)-2-(4-(2-cyano-4,4-dimethylpent-2-enoyl)piperazin-1-yl) acetic acid (0.07 g, 28%) as pale yellow semi solid.

¹H NMR (400 MHz, DMSO-d₆) δ=6.85 (s, 1H), 4.00-3.94 (m, 1H), 3.77-3.57 (m, 4H), 3.50-3.35 (m, 4H), 1.23 (s, 9H), 1.19 (s, 1H), 1.08 (s, 1H)

LC-MS: 97.47% 280.2 [M+H]⁺ at 1.503 min; Column: X-Select CSH C18 (3.0*50 mm, 2.5 um) Mobile Phase A: 0.05% FA in H2O, Mobile Phase B: 0.05% FA in ACN, Gradient % B: 0/2,0.3/2,2.0/98,3.0/98,3.2/2,3.5/2 Flow Rate: 1.0 ml/min

HPLC: 86.79% at RT: 5.055 min, Column: X-Select CSH C18 (4.6*150) mm 5u Mobile Phase: A-0.1% Formic acid in water: Acetonitrile (95:05) B-Acetonitrile Flow Rate: 1.0. mL/minute Gradient program: Time (min)/B Conc: 0.01/5, 1.0/5, 8.0/100, 12.0/100, 14.0/5, 18.0/5

### Synthesis of tert-butyl 5-hydroxy-1-methyl-3,4-dihydroisoquinoline-2(1H)-carboxylate

A stirred solution of tert-butyl 5-bromo-1-methyl-3,4-dihydroisoquinoline-2(1H)-carboxylate (4.0 g, 12.2 mmol), BuXPhos-Pd-G₃ (0.48 g, 0.61 mmol) and KOH (73.6 mL, 73.6 mmol) in 1,4-dioxane (40 mL) was heated at 110° C. for 2 h. The reaction mixture was diluted with water and extracted with EtOAc. The organic layer was dried over anhydrous Na₂SO₄ and concentrated in vacuo. The resulting residue was purified by silica gel column chromatography (gradient=20-30% EtOAc in n-hexane) to afford tert-butyl 5-hydroxy-1-methyl-3,4-dihydroisoquinoline-2(1H)-carboxylate (2.3 g, 8.73 mmol, 71.2% yield). LCMS m/z=208.0 [M+H]⁺.

### Exemplary Bifunctional Compounds:

TABLE 1b
The following examples were prepared following general procedure 1b using starting
materials as indicated in Table 2b.

| Compound | |
|---|---|
| No. | Structure |
| SMARCA2a | 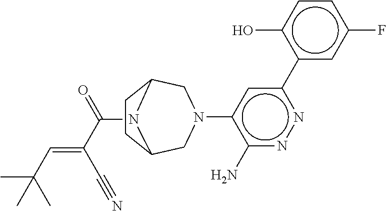 |
| SMARCA2b | 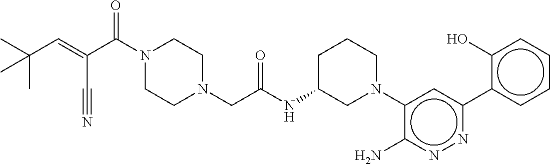 |
| SMARCA2c | 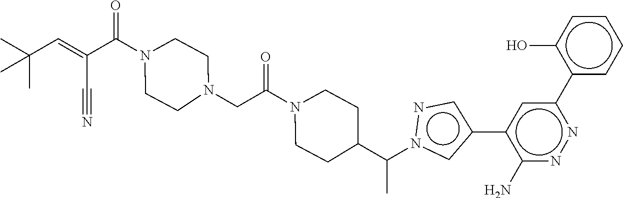 |
| SMARCA2d | 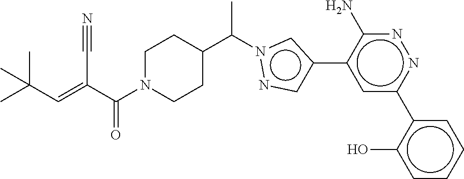 |
| SMARCA2e | 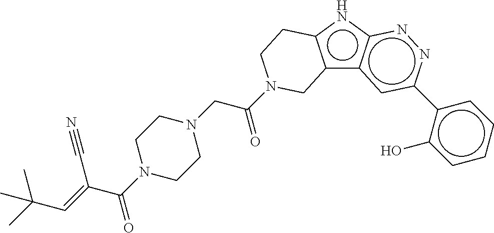 |
| SMARCA2f | 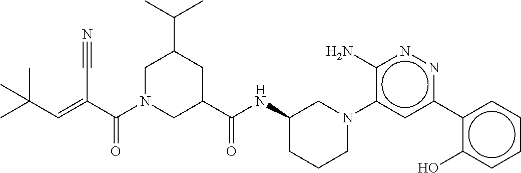 |
| SMARCA2g | 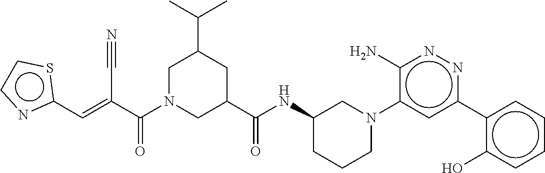 |
| SMARCA2h | 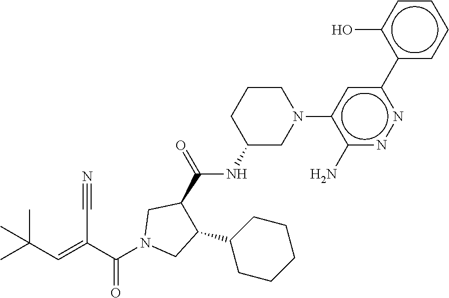 |
| SMARCA2i | 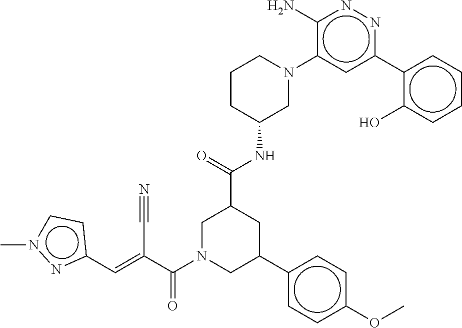 |
| SMARCA2j | 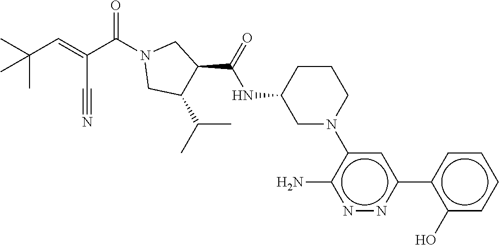 |
| SMARCA2k | 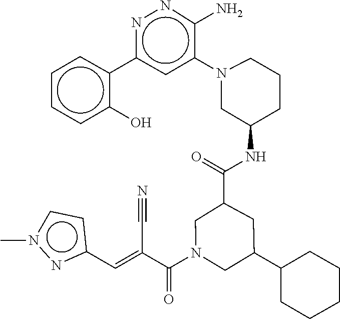 |
| SMARCA21 | 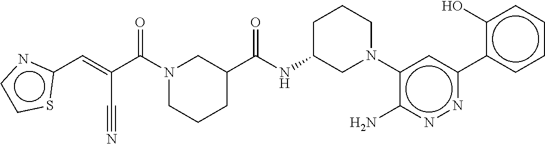 |

TABLE 2b
The following table indicates the intermediates used to prepare
the indicated examples, following general procedure 1b.

| Compound | | |
|---|---|---|
| No. | Intermediate A | Intermediate B |
| SMARCA2a | (E/Z)-2-cyano-4,4- | 2-(6-amino-5-(3,8- |
| | dimethyl-pent-2- | diazabicyclo[3.2.1]octan- |
| | enoic acid | 3-yl)pyridazin-3-yl)-4- |
| | | fluorophenol |
| SMARCA2b | (E)-2-(4-(2-cyano-4,4- | (R)-2-(6-amino-5-(3- |
| | dimethylpent-2- | aminopiperidin-1- |
| | enoyl)piperazin-1- | yl)pyridazin-3-yl)phenol |
| | yl)acetic acid | |
| SMARCA2c | (E)-2-(4-(2-cyano-4,4- | 2-(6-amino-5-(1-(1- |
| | dimethylpent-2- | (piperidin-4-yl)ethyl)-1H- |
| | enoyl)piperazin-1- | pyrazol-4-yl)pyridazin-3- |
| | yl)acetic acid | yl)phenol |
| SMARCA2d | (E/Z)-2-cyano-4,4- | 2-(6-amino-5-(1-(1- |
| | dimethyl-pent-2- | (piperidin-4-yl)ethyl)-1H- |
| | enoic acid | pyrazol-4-yl)pyridazin-3- |
| | | yl)phenol |
| SMARCA2e | (E)-2-(4-(2-cyano-4,4- | 2-(6,7,8,9-tetrahydro-5H- |
| | dimethylpent-2- | pyrido[3′,4′:4,5]pyrrolo[2,3- |
| | enoyl)piperazin-1- | c]pyridazin-3-yl)phenol |
| | yl)acetic acid | |
| SMARCA2f | Pipacid 16 | (R)-2-(6-amino-5-(3- |
| | | aminopiperidin-1- |
| | | yl)pyridazin-3-yl)phenol |
| SMARCA2g | Pipacid 15 | (R)-2-(6-amino-5-(3- |
| | | aminopiperidin-1- |
| | | yl)pyridazin-3-yl)phenol |
| SMARCA2h | Pyracid 29 | (R)-2-(6-amino-5-(3- |
| | | aminopiperidin-1- |
| | | yl)pyridazin-3-yl)phenol |
| SMARCA2i | Pipacid 27 | (R)-2-(6-amino-5-(3- |
| | | aminopiperidin-1- |
| | | yl)pyridazin-3-yl)phenol |
| SMARCA2j | Pyracid 46 | (R)-2-(6-amino-5-(3- |
| | | aminopiperidin-1- |
| | | yl)pyridazin-3-yl)phenol |
| SMARCA2k | Pipacid 22 | (R)-2-(6-amino-5-(3- |
| | | aminopiperidin-1- |
| | | yl)pyridazin-3-yl)phenol |
| SMARCA2l | Pipacid 5 | (R)-2-(6-amino-5-(3- |
| | | aminopiperidin-1- |
| | | yl)pyridazin-3-yl)phenol |

### PART B—Biological Data

The bifunctional compounds were assayed to investigate their ability to degrade target proteins in accordance with the following general procedures.

### Example 1—BET (BRD4) Degradation

### 1.1 Assay 1-Degradation of HiBit-BRD4 in HEK293

HEK293 containing a HiBit insertion for BRD4 were plated in 384-well tissue culture plates at a density of 8×10⁴ per well in a volume of 36 μL and incubated overnight at 37° C. and 5% CO₂. Wells were treated with test compounds for 6 h prior to addition of the NanoLuc substrate and reading on a ClarioSLARIOstar Plus. Degradation data was plotted and analysed using Prism 86 (Graphpad).

The degradation of target protein BRD4 was detected according to the procedure outlined in assay 1 (Degradation of HiBit-BRD4 in HEK293) for the following compounds.

| | Structure |
|---|---|
| 8a | 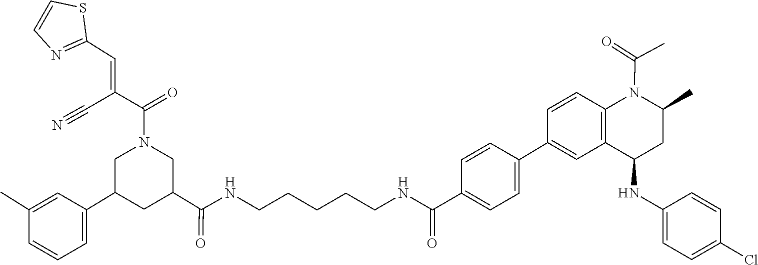 [SMILES: CC(=O)N1c2ccc(-c3ccc(C(=O)NCCCCCNC(=O)C4CC(c5cccc(C)c5)CN(C(=O)/C(C#N)=C/c5nccs5)C4)cc3)cc2[C@H](Nc2ccc(Cl)cc2)C[C@@H]1C] |
| 8b | 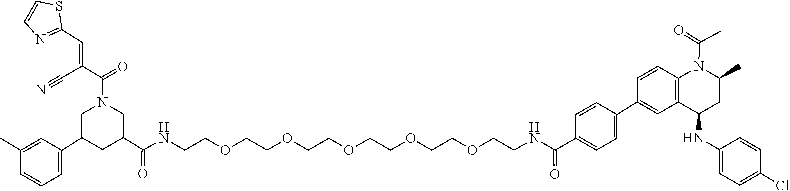 [SMILES: CC(=O)N1c2ccc(-c3ccc(C(=O)NCCOCCOCCOCCOCCOCCNC(=O)C4CC(c5cccc(C)c5)CN(C(=O)/C(C#N)=C/c5nccs5)C4)cc3)cc2[C@H](Nc2ccc(Cl)cc2)C[C@@H]1C] |
| 8d | 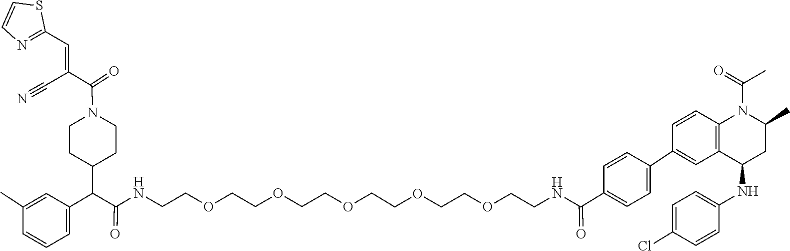 |
| 8c | 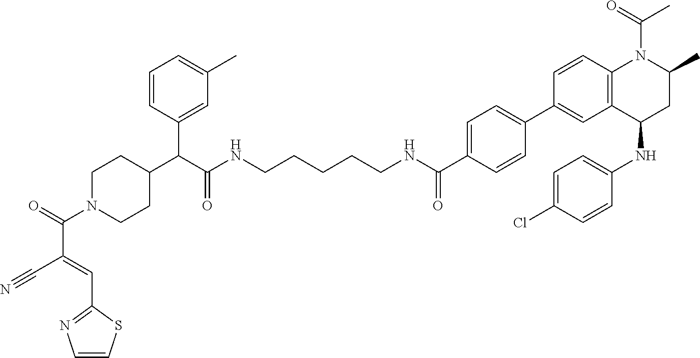 [SMILES: CC(=O)N1c2ccc(-c3ccc(C(=O)NCCCCCNC(=O)C(c4cccc(C)c4)C4CCN(C(=O)/C(C#N)=C/c5nccs5)CC4)cc3)cc2[C@H](Nc2ccc(Cl)cc2)C[C@@H]1C] |
| 8e | 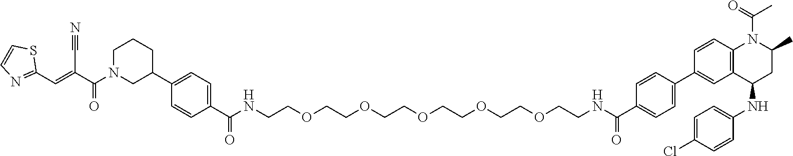 [SMILES: CC(=O)N1c2ccc(-c3ccc(C(=O)NCCOCCOCCOCCOCCOCCNC(=O)c4ccc(C5CCCN(C(=O)/C(C#N)=C/c6nccs6)C5)cc4)cc3)cc2[C@H](Nc2ccc(Cl)cc2)C[C@@H]1C] |
| 8h | 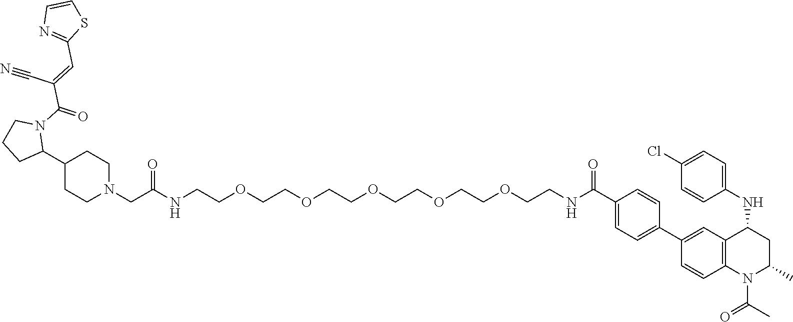 [SMILES: CC(=O)N1c2ccc(-c3ccc(C(=O)NCCOCCOCCOCCOCCOCCNC(=O)CN4CCC(C5CCCN5C(=O)/C(C#N)=C/c5nccs5)CC4)cc3)cc2[C@H](Nc2ccc(Cl)cc2)C[C@@H]1C] |
| 8f | 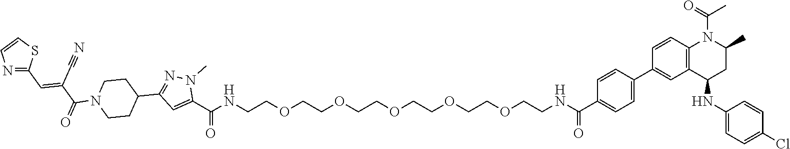 [SMILES: CC(=O)N1c2ccc(-c3ccc(C(=O)NCCOCCOCCOCCOCCOCCNC(=O)c4cc(C5CCN(C(=O)/C(C#N)=C/c6nccs6)CC5)nn4C)cc3)cc2[C@H](Nc2ccc(Cl)cc2)C[C@@H]1C] |
| 8g | 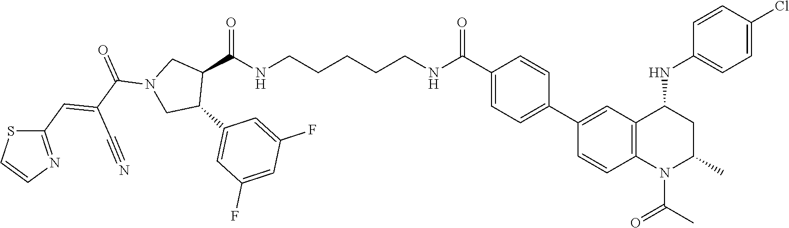 [SMILES: CC(=O)N1c2ccc(-c3ccc(C(=O)NCCCCCNC(=O)[C@@H]4CN(C(=O)/C(C#N)=C/c5nccs5)C[C@H]4c4cc(F)cc(F)c4)cc3)cc2[C@H](Nc2ccc(Cl)cc2)C[C@@H]1C] |

Table 1.1 shows the bifunctional molecules that were analysed in accordance with the procedure outlined in assay 1.

The DC₅₀ values for all of the compounds shown in Table 1.1 were all found to be less than or equal to 1000 nM. These molecules were all considered to be effective degraders.

The DC₅₀ values for compounds 8a, 8b, 8e, 8f, 8g, and 8 h was found to be less than 500 nM. These molecules were considered to be particularly effective degraders.

Degradation of target protein BRD4 was also detected according to the procedure outlined in assay 1 (Degradation of HiBit-BRD4 in HEK293) for the following compounds:

| Ref. | DC₅₀ | Dₘₐₓ |
|---|---|---|
| 13a | ++ | +++ |
| 13b | ++ | +++ |
| 13c | ++ | NA |
| 13d | + | + |
| 13e | + | + |
| 13f | + | ++ |
| 13g | + | ++ |
| 13i | + | + |
| 13k | +++ | +++ |
| 13l | + | + |
| 13m | ++ | ++ |
| 13n | + | ++ |
| 13p | +++ | +++ |
| 13q | ++ | ++ |
| 13r | ++ | +++ |
| 13s | ++ | +++ |
| 13t | ++ | +++ |
| 13u | +++ | +++ |
| 13v | ++ | +++ |
| 13x | ++ | +++ |
| 13z | +++ | +++ |
| 13aa | + | +++ |
| 13ab | + | ++ |
| 13ac | ++ | +++ |
| 13ae | ++ | +++ |
| 13af | ++ | ++ |
| 13ag | ++ | ++ |
| 13ah | ++ | +++ |
| 13ai | + | + |
| 13ak | ++ | +++ |
| 13an | ++ | + |
| 13at | ++ | +++ |
| 13au | ++ | +++ |
| 13av | + | ++ |
| 13aw | + | NA |
| 13ax | +++ | +++ |

NA = degradation does not reach 50%
DC₅₀ key: + = ≥200 nM, ++ = ≥25 nM and <200 nM, +++ = <25 nM
Dₘₐₓ key: + = ≥30% and <50%, ++ = ≥50% and <70%, +++ = ≥70%.

Degradation of target protein BRD4 was also detected according to the procedure outlined in assay 1 (Degradation of HiBit-BRD4 in HEK293) for the following compounds:

| Ref. | DC₅₀ | Dₘₐₓ |
|---|---|---|
| 14a | ++ | +++ |

DC₅₀ key: + = ≥200 nM, ++ = ≥25 nM and <200 nM, +++ = <25 nM
Dₘₐₓ key: + = ≥30% and <50%, ++ = ≥50% and <70%, +++ = ≥70%.

Degradation of target protein BRD4 was also detected according to the procedure outlined in assay 1 (Degradation of HiBit-BRD4 in HEK293) for the following compounds:

| Ref. | DC₅₀ | Dₘₐₓ |
|---|---|---|
| 15b | ++ | +++ |
| 15f | + | + |
| 15g | ++ | +++ |
| 15h | ++ | ++ |
| 15i | + | ++ |
| 15k | ++ | +++ |
| 15l | ++ | +++ |
| 15m | ++ | ++ |
| 15o | ++ | ++ |
| 15p | ++ | +++ |
| 15q | ++ | +++ |
| 15r | + | ++ |
| 15s | ++ | ++ |
| 15t | ++ | +++ |
| 15u | ++ | ++ |
| 15w | ++ | + |
| 15x | + | + |
| 15z | ++ | + |
| 15aa | ++ | +++ |
| 15ab | ++ | ++ |
| 15ac | + | NA |
| 15ad | + | NA |
| 15ae | ++ | +++ |
| 15af | ++ | +++ |
| 15ag | ++ | + |
| 15ah | + | + |
| 15ai | ++ | + |
| 15aj | ++ | ++ |
| 15ak | ++ | +++ |
| 15al | + | +++ |
| 15an | ++ | + |

NA = degradation does not reach 50%
DC₅₀ key: + = ≥200 nM, ++ = ≥25 nM and <200 nM, +++ = <25 nM
Dₘₐₓ key: + = ≥30% and <50%, ++ = ≥50% and <70%, +++ = ≥70%.

### Example 2—BRD9 Degradation

### Assay Protocol 1—Degradation of HiBit-BRD9 in HEK293

HEK293 stably expressing LgBit and containing a HiBit insertion for BRD9 were plated in 384-well tissue culture plates at a density of 8×10³ per well in a volume of 36 μL and incubated overnight at 37° C. and 5% CO₂. Wells were treated with test compounds (11 pt titration up to 10 μM) for 6 h prior to addition of Nano-Glo® Hibit Lytic buffer (1:50 substrate and 1:100 LgBit protein) and reading on a PheraSTAR. Degradation data was plotted and analysed using Prism 8 (Graphpad).

### Assay Protocol 2—Degradation of BRD9 (IF)

A suspension of MV4-11 cells (ATCC CRL-9591) was prepared in phenol red-free assay media (IMDM Thermo Scientific 21056023+10% FBS ATCC 302025) and cells were seeded at 20,000 cells per well (45 μL) in sterile black poly-d-lysine coated 384 well plates (Greiner 781948). Compounds were prepared at 1000× final concentration in DMSO, diluted 1:100 in assay media, and 5 μL compound was added to each well of the cell plate. Cells were incubated for 24 hours at 37° C. with 5% CO₂. All the following incubations for immunofluorescence staining were at room temperature. 15 μL of 16% PFA was added to each well (3.7% final concentration) and the cells were fixed for 15 min then washed twice with DPBS. Cells were permeabilised with 0.1% Triton X-100 for 10 min, Triton X-100 was removed, then blocked with 1% BSA in DPBS for 1 hour. Cells were stained with 25 μL anti-BRD9 E4Q3F antibody (CST 48306) diluted 1:25600 in 1% BSA in DPBS for 2-3 hours. Wells were washed twice with DPBS then incubated with 25 μL of 1% BSA containing a 1:1000 dilution of Anti-rabbit Alexa Fluor™ 647 secondary antibody (Thermo Scientific A²¹²⁴⁴) and 1 μg/mL Hoechst nuclear counter stain (Abcam ab228551) for 1 hour. Wells were washed twice with DPBS prior to imaging on a Perkin Elmer Operetta CLS with 10× air lens. Images were processed using Harmony High-Content Imaging and Analysis Software (Perkin Elmer) and the mean contrast ratio of Alexa Fluor™ 647 in central nuclei was used to quantify BRD9 protein levels. Data was further analysed using Dotmatics software and % BRD9 remaining was calculated by normalisation to average data from high and low control wells (cells treated with DMSO or 100 nM CFT-8634 respectively).

### Degradation Results

The degradation of BRD9 was detected according to the procedure outlined in the assay protocol above for a number of exemplary bifunctional molecules. The results are shown in Table I below.

TABLE I

| | | DC | Dma | DC | Dma |
|---|---|---|---|---|---|
| Ref. | Structure | 50¹ | x¹ | 50² | x² |
| BRD9a | 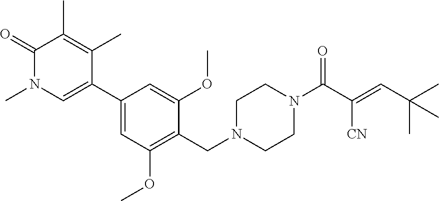 [SMILES: COc1cc(-c2cn(C)c(=O)c(C)c2C)cc(OC)c1CN1CCN(C(=O)/C(C#N)=C/C(C)(C)C)CC1] | ++ | ++ | +++ | ++ |
| BRD9b | 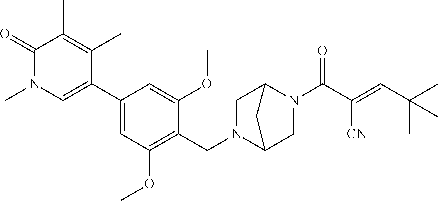 [SMILES: COc1cc(-c2cn(C)c(=O)c(C)c2C)cc(OC)c1CN1CC2CC1CN2C(=O)/C(C#N)=C/C(C)(C)C] | +++ | +++ | + | ++ |
| BRD9c | 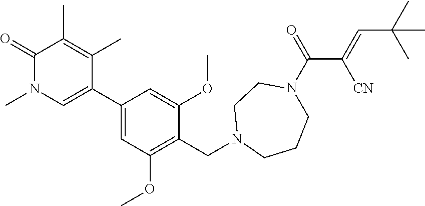 [SMILES: COc1cc(-c2cn(C)c(=O)c(C)c2C)cc(OC)c1CN1CCCN(C(=O)/C(C#N)=C/C(C)(C)C)CC1] | +++ | +++ | +++ | +++ |
| BRD9d | 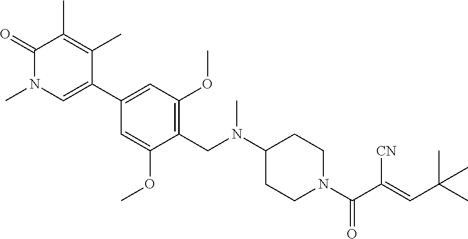 [SMILES: COc1cc(-c2cn(C)c(=O)c(C)c2C)cc(OC)c1CN(C)C1CCN(C(=O)/C(C#N)=C/C(C)(C)C)CC1] | +++ | +++ | +++ | ++ |
| BRD9e | 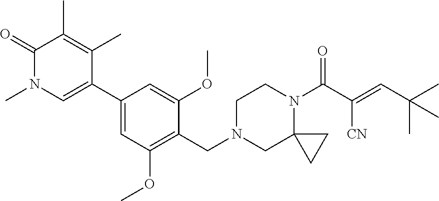 [SMILES: COc1cc(-c2cn(C)c(=O)c(C)c2C)cc(OC)c1CN1CCN(C(=O)/C(C#N)=C/C(C)(C)C)C2(CC2)C1] | | | | + |
| BRD9f | 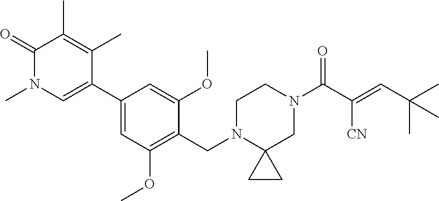 [SMILES: COc1cc(-c2cn(C)c(=O)c(C)c2C)cc(OC)c1CN1CCN(C(=O)/C(C#N)=C/C(C)(C)C)CC12CC2] | | | +++ | ++ |
| BRD9g | 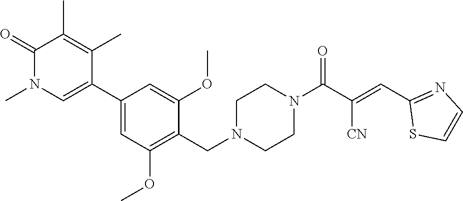 [SMILES: COc1cc(-c2cn(C)c(=O)c(C)c2C)cc(OC)c1CN1CCN(C(=O)/C(C#N)=C/c2nccs2)CC1] | + | + | | |
| BRD9h | 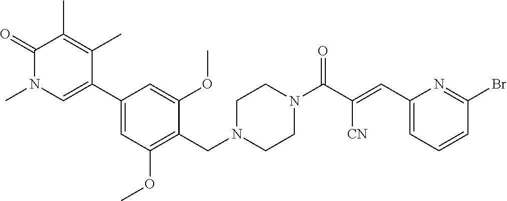 [SMILES: COc1cc(-c2cn(C)c(=O)c(C)c2C)cc(OC)c1CN1CCN(C(=O)/C(C#N)=C/c2cccc(Br)n2)CC1] | +++ | +++ | +++ | +++ |
| BRD9i | 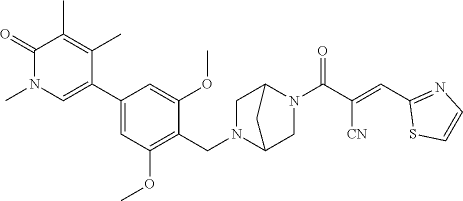 [SMILES: COc1cc(-c2cn(C)c(=O)c(C)c2C)cc(OC)c1CN1CC2CC1CN2C(=O)/C(C#N)=C/c1nccs1] | ++ | ++ | + | + |
| BRD9j | 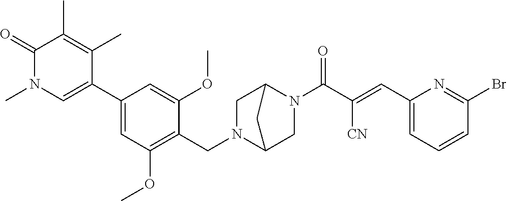 [SMILES: COc1cc(-c2cn(C)c(=O)c(C)c2C)cc(OC)c1CN1CC2CC1CN2C(=O)/C(C#N)=C/c1cccc(Br)n1] | | | +++ | +++ |
| BRD9k | 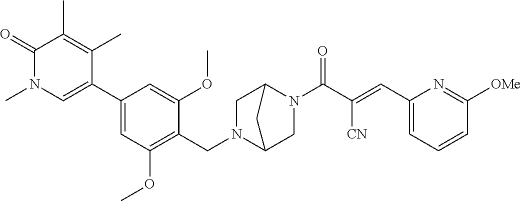 [SMILES: COc1cccc(/C=C(\C#N)C(=O)N2CC3CC2CN3Cc2c(OC)cc(-c3cn(C)c(=O)c(C)c3C)cc2OC)n1] | | | +++ | ++ |
| BRD9l | 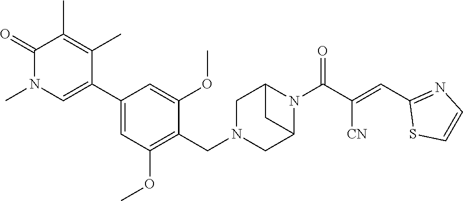 [SMILES: COc1cc(-c2cn(C)c(=O)c(C)c2C)cc(OC)c1CN1CC2CC(C1)N2C(=O)/C(C#N)=C/c1nccs1] | + | ++ | | + |
| BRD9m | 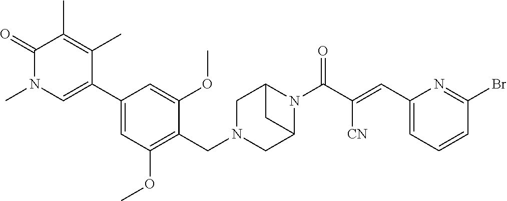 [SMILES: COc1cc(-c2cn(C)c(=O)c(C)c2C)cc(OC)c1CN1CC2CC(C1)N2C(=O)/C(C#N)=C/c1cccc(Br)n1] | +++ | +++ | +++ | +++ |
| BRD9n | 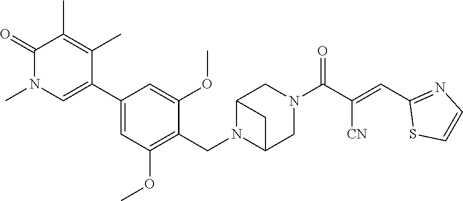 [SMILES: COc1cc(-c2cn(C)c(=O)c(C)c2C)cc(OC)c1CN1C2CC1CN(C(=O)/C(C#N)=C/c1nccs1)C2] | +++ | ++ | +++ | +++ |
| BRD9o | 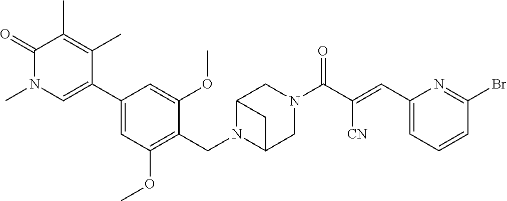 [SMILES: COc1cc(-c2cn(C)c(=O)c(C)c2C)cc(OC)c1CN1C2CC1CN(C(=O)/C(C#N)=C/c1cccc(Br)n1)C2] | +++ | +++ | +++ | +++ |
| BRD9p | 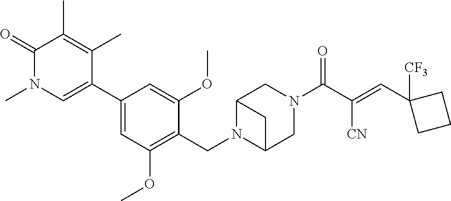 [SMILES: COc1cc(-c2cn(C)c(=O)c(C)c2C)cc(OC)c1CN1C2CC1CN(C(=O)/C(C#N)=C/C1(C(F)(F)F)CCC1)C2] | | | ++ | ++ |
| BRD9q | 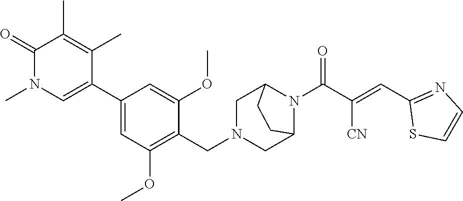 [SMILES: COc1cc(-c2cn(C)c(=O)c(C)c2C)cc(OC)c1CN1CC2CCC(C1)N2C(=O)/C(C#N)=C/c1nccs1] | + | ++ | + | ++ |
| BRD9r | 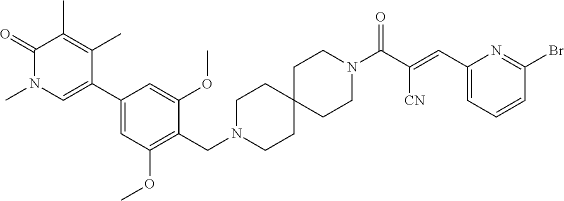 [SMILES: COc1cc(-c2cn(C)c(=O)c(C)c2C)cc(OC)c1CN1CCC2(CC1)CCN(C(=O)/C(C#N)=C/c1cccc(Br)n1)CC2] | +++ | +++ | +++ | +++ |
| BRD9s | 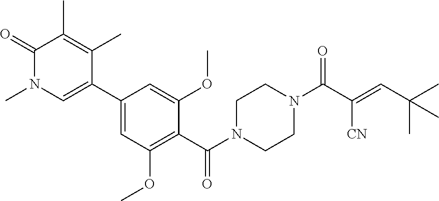 [SMILES: COc1cc(-c2cn(C)c(=O)c(C)c2C)cc(OC)c1C(=O)N1CCN(C(=O)/C(C#N)=C/C(C)(C)C)CC1] | | | ++ | ++ |
| BRD9t | 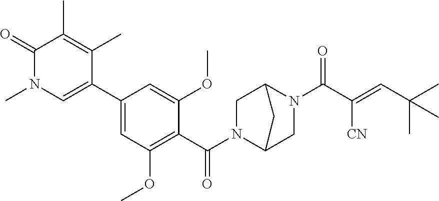 [SMILES: COc1cc(-c2cn(C)c(=O)c(C)c2C)cc(OC)c1C(=O)N1CC2CC1CN2C(=O)/C(C#N)=C/C(C)(C)C] | | | ++ | +++ |
| BRD9u | 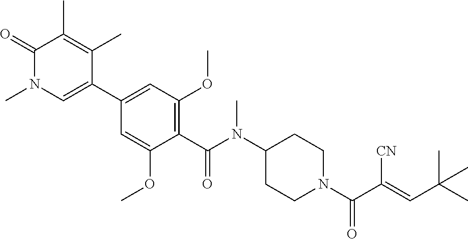 [SMILES: COc1cc(-c2cn(C)c(=O)c(C)c2C)cc(OC)c1C(=O)N(C)C1CCN(C(=O)/C(C#N)=C/C(C)(C)C)CC1] | | | | + |
| BRD9v | 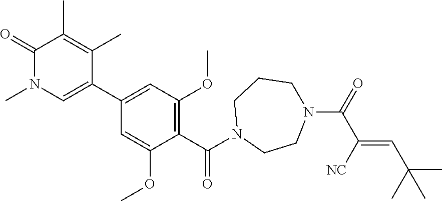 [SMILES: COc1cc(-c2cn(C)c(=O)c(C)c2C)cc(OC)c1C(=O)N1CCCN(C(=O)/C(C#N)=C/C(C)(C)C)CC1] | | | ++ | ++ |
| BRD9w | 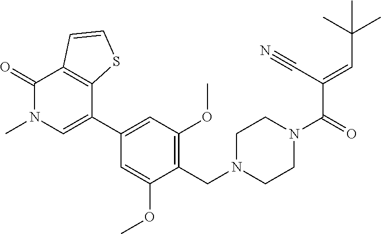 [SMILES: COc1cc(-c2cn(C)c(=O)c3ccsc23)cc(OC)c1CN1CCN(C(=O)/C(C#N)=C/C(C)(C)C)CC1] | ++ | +++ | | +++ |
| BRD9x | 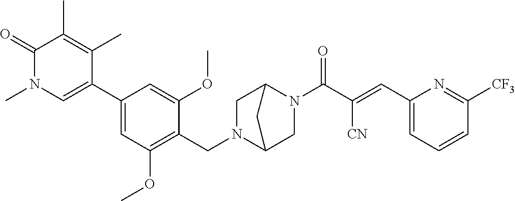 [SMILES: COc1cc(-c2cn(C)c(=O)c(C)c2C)cc(OC)c1CN1CC2CC1CN2C(=O)/C(C#N)=C/c1cccc(C(F)(F)F)n1] | | | +++ | +++ |
| BRD9y | 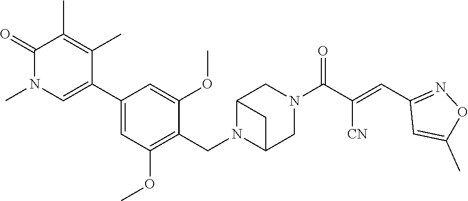 [SMILES: COc1cc(-c2cn(C)c(=O)c(C)c2C)cc(OC)c1CN1C2CC1CN(C(=O)/C(C#N)=C/c1cc(C)on1)C2] | | | | + |
| BRD9z | 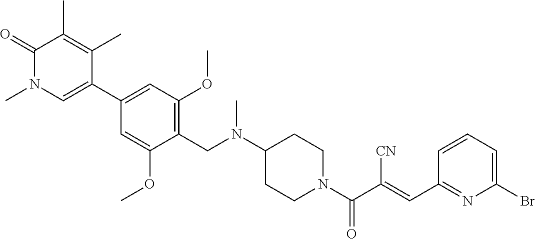 [SMILES: COc1cc(-c2cn(C)c(=O)c(C)c2C)cc(OC)c1CN(C)C1CCN(C(=O)/C(C#N)=C/c2cccc(Br)n2)CC1] | +++ | +++ | +++ | +++ |
| BRD9aa | 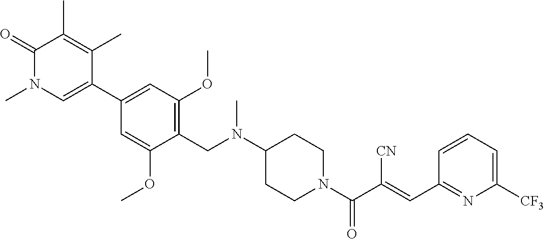 [SMILES: COc1cc(-c2cn(C)c(=O)c(C)c2C)cc(OC)c1CN(C)C1CCN(C(=O)/C(C#N)=C/c2cccc(C(F)(F)F)n2)CC1] | | | | +++ |
| BRD9ad | 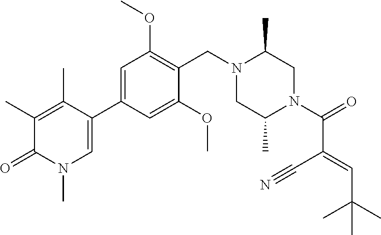 [SMILES: COc1cc(-c2cn(C)c(=O)c(C)c2C)cc(OC)c1CN1C[C@@H](C)N(C(=O)/C(C#N)=C/C(C)(C)C)C[C@@H]1C] | | | ++ | ++ |
| BRD9ae | 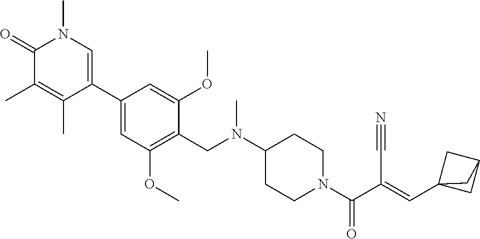 [SMILES: COc1cc(-c2cn(C)c(=O)c(C)c2C)cc(OC)c1CN(C)C1CCN(C(=O)/C(C#N)=C/C23CC(C2)C3)CC1] | +++ | +++ | +++ | +++ |
| BRD9af | 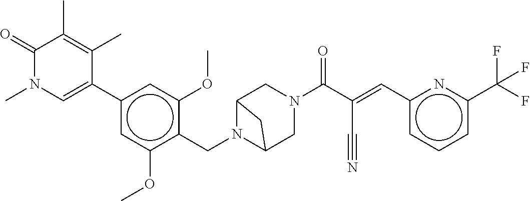 | | | +++ | +++ |
| BRD9ag | 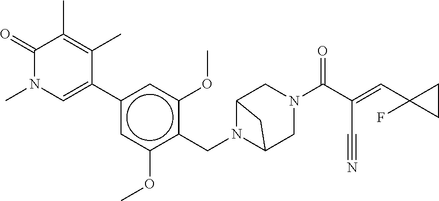 | | | +++ | +++ |
| BRD9ah | 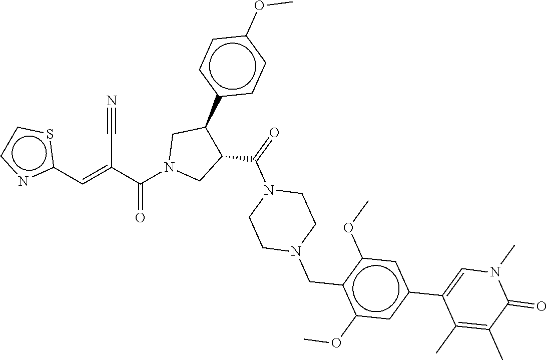 | | + | +++ | +++ |
| BRD9ai | 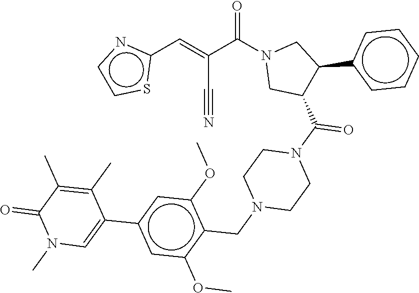 | | + | | ++ |
| BRD9ba | 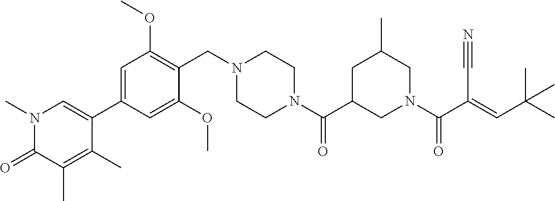 [SMILES: COc1cc(-c2cn(C)c(=O)c(C)c2C)cc(OC)c1CN1CCN(C(=O)C2CC(C)CN(C(=O)/C(C#N)=C/C(C)(C)C)C2)CC1] | | | +++ | +++ |
| BRD9bb | 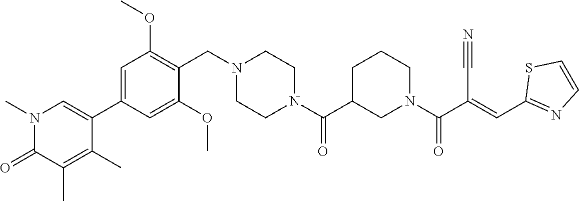 [SMILES: COc1cc(-c2cn(C)c(=O)c(C)c2C)cc(OC)c1CN1CCN(C(=O)C2CCCN(C(=O)/C(C#N)=C/c3nccs3)C2)CC1] | | + | ++ | ++ |
| BRD9bc | 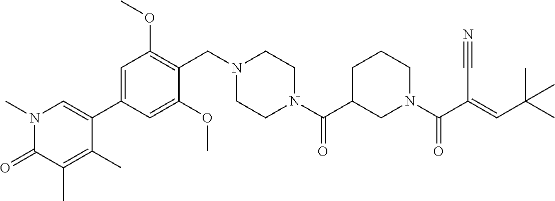 [SMILES: COc1cc(-c2cn(C)c(=O)c(C)c2C)cc(OC)c1CN1CCN(C(=O)C2CCCN(C(=O)/C(C#N)=C/C(C)(C)C)C2)CC1] | ++ | +++ | ++ | ++ |
| BRD9bd | 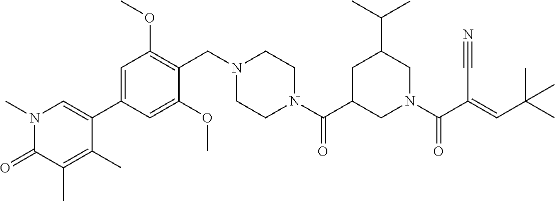 [SMILES: COc1cc(-c2cn(C)c(=O)c(C)c2C)cc(OC)c1CN1CCN(C(=O)C2CC(C(C)C)CN(C(=O)/C(C#N)=C/C(C)(C)C)C2)CC1] | ++ | +++ | ++ | +++ |
| BRD9be | 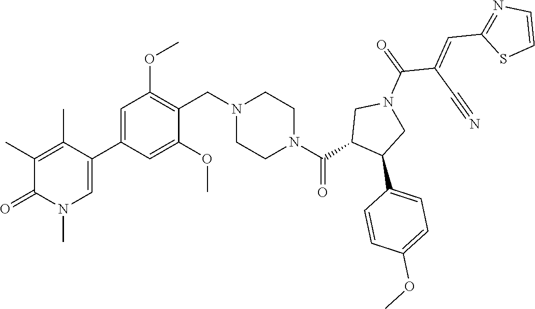 [SMILES: COc1ccc([C@@H]2CN(C(=O)/C(C#N)=C/c3nccs3)C[C@H]2C(=O)N2CCN(Cc3c(OC)cc(-c4cn(C)c(=O)c(C)c4C)cc3OC)CC2)cc1] | | ++ | +++ | +++ |
| BRD9bf | 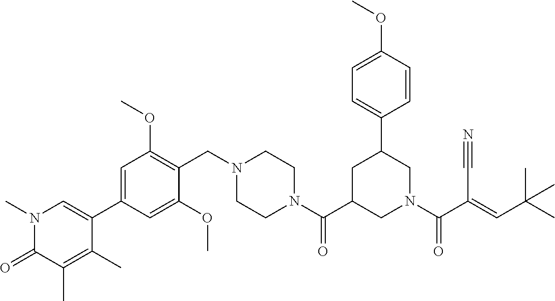 [SMILES: COc1ccc(C2CC(C(=O)N3CCN(Cc4c(OC)cc(-c5cn(C)c(=O)c(C)c5C)cc4OC)CC3)CN(C(=O)/C(C#N)=C/C(C)(C)C)C2)cc1] | +++ | +++ | +++ | +++ |
| BRD9bg | 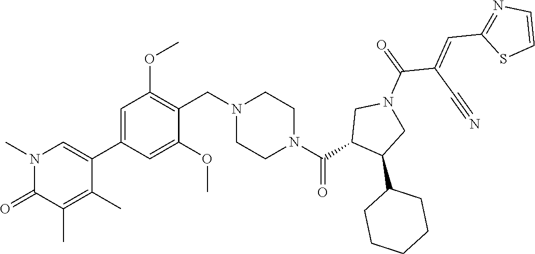 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2C2CCCCC2)CC1] | + | + | ++ | +++ |
| BRD9bh | 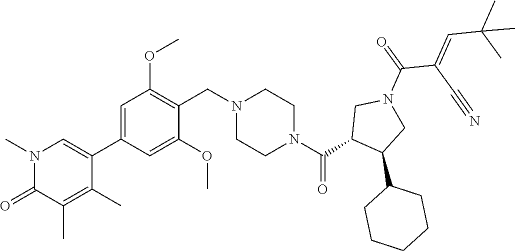 [SMILES: COc1cc(-c2cn(C)c(=O)c(C)c2C)cc(OC)c1CN1CCN(C(=O)[C@@H]2CN(C(=O)/C(C#N)=C/C(C)(C)C)C[C@H]2C2CCCCC2)CC1] | ++ | ++ | ++ | ++ |
| BRD9bi | 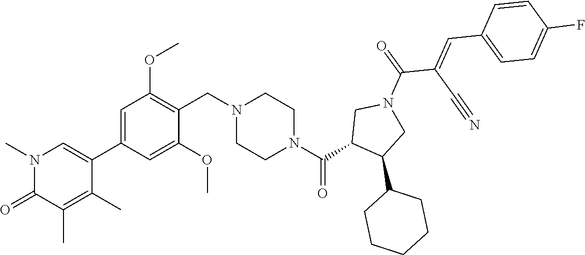 [SMILES: COc1cc(-c2cn(C)c(=O)c(C)c2C)cc(OC)c1CN1CCN(C(=O)[C@@H]2CN(C(=O)/C(C#N)=C/c3ccc(F)cc3)C[C@H]2C2CCCCC2)CC1] | + | +++ | + | ++ |
| BRD9bj | 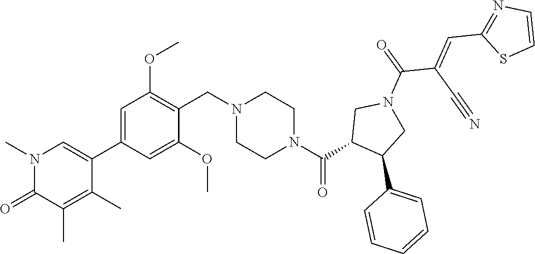 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2c2ccccc2)CC1] | | + | | ++ |
| BRD9bk | 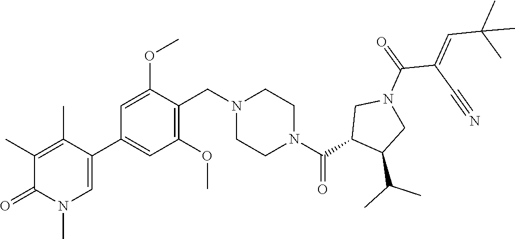 [SMILES: COc1cc(-c2cn(C)c(=O)c(C)c2C)cc(OC)c1CN1CCN(C(=O)[C@@H]2CN(C(=O)/C(C#N)=C/C(C)(C)C)C[C@H]2C(C)C)CC1] | ++ | ++ | ++ | ++ |
| BRD9bl | 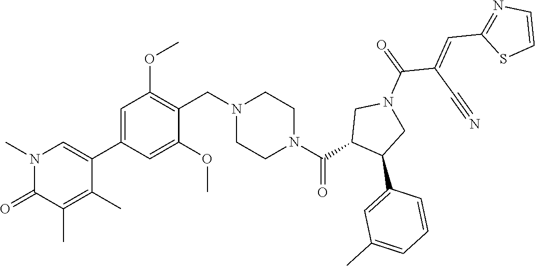 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2c2cccc(C)c2)CC1] | | + | + | ++ |
| BRD9bm | 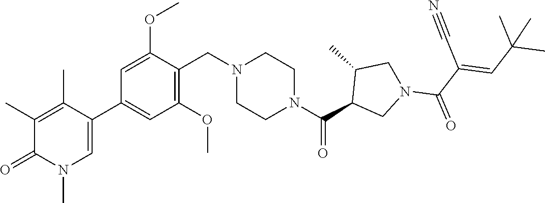 [SMILES: COc1cc(-c2cn(C)c(=O)c(C)c2C)cc(OC)c1CN1CCN(C(=O)[C@@H]2CN(C(=O)/C(C#N)=C/C(C)(C)C)C[C@H]2C)CC1] | | | + | ++ |

DC₅₀ key: + = ≥200 nM, ++ = ≥25 nM and <200 nM, +++ = <25 nM
Dₘₐₓ key: + = ≥30% and <50%, ++ = ≥50% and <70%, +++ = ≥70%.
¹ = determined according to Assay Protocol 1.
² = determined according to Assay Protocol 2.

### Example 3—SMARCA2 Degradation

The bifunctional compounds were assayed to investigate their ability to degrade target proteins in accordance with the following general procedures.

### Assay Protocol 1

Hela cells with a HiBiT tag knocked in at the 3′ end of SMARCA2 were purchased from Promega (Custom product) (1). HeLa SMARCA2 HiBiT transfected cells were treated with compounds at indicated concentrations using the FlexDrop IQ (Perkin Elmer). 24 hours after compound additions abundance of HiBiT tagged protein was quantified using NanoGlo HiBiT lytic detection system (Promega, N3050). Cell viability was measured using Cell Titre Glow cell viability assay (Promega, G9243). In each case luminescence was measured on a Pherastar platereader.

1 Schwinn M K, Machleidt T, Zimmerman K, Eggers C T, Dixon A S, Hurst R, Hall M P, Encell L P, Binkowski B F, Wood K V. CRISPR-Mediated Tagging of Endogenous Proteins with a Luminescent Peptide. ACS Chem Biol. 2018 Feb. 16;13 (2): 467-474. doi: 10.1021/acschembio.7b00549. Epub 2017 Sep. 21. PMID: 28892606.

### SMARCA2 Degradation Results

The degradation of SMARCA2 was detected according to the procedure outlined in the Assay Protocol for a number of exemplary bifunctional molecules. The results are shown in Table SMARCA2-1 below.

| Compound | Dₘₐₓ |
|---|---|
| SMARCA2a | N/A |
| SMARCA2b | + |
| SMARCA2c | N/A |
| SMARCA2d | N/A |
| SMARCA2e | + |
| SMARCA2f | + |
| SMARCA2g | ++ |
| SMARCA2h | ++ |
| SMARCA2i | + |
| SMARCA2j | ++ |
| SMARCA2k | N/A |
| SMARCA2l | + |

Key: NA = degradation does not reach 50%;
Dₘₐₓ Key: + = >50% and <65%, ++ = ≥65 and <80%, +++ = ≥80%

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A bifunctional molecule comprising the general formula: 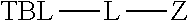
wherein TBL is a target protein binding ligand;
L is a linker; and
Z comprises a structure according to formula (ZI): 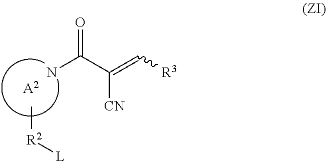
wherein:
ring A² is an optionally substituted 4- to 7-membered monocyclic N-heterocycloalkyl or an optionally substituted 7- to 12-membered bicyclic N-heterocycloalkyl, each optionally containing one or two additional ring heteroatoms selected from N, O and S;
R² is absent or is selected from the group consisting of aryl, substituted aryl, heteroaryl, substituted heteroaryl, heterocycloalkyl, substituted heterocycloalkyl, NR^{y}, —CH(aryl)-, CH(substituted aryl)-, —CH(heteroaryl)- and —CH(substituted heteroaryl)-;
R^{y} is optionally substituted C₁₋₆alkyl or H;
R³ is selected from the group consisting of C₁₋₆ alkyl, cycloalkyl, substituted cycloalkyl, alkylcycloalkyl, substituted alkylcycloalkyl, alkyl heterocycloalkyl, substituted alkylcycloalkyl, heterocycloalkyl, substituted heterocycloalkyl, aryl, substituted aryl, heteroaryl, and substituted heteroaryl, wherein the C₁₋₆ alkyl is optionally substituted with one or more halo, N, O and S; and
L shows the point of attachment of the linker;
and further wherein Z is not: 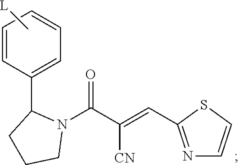
or a pharmaceutically acceptable salt thereof.

2-5. (canceled)

6. The bifunctional molecule of claim 1, wherein Z comprises a structure according to formula (ZII): 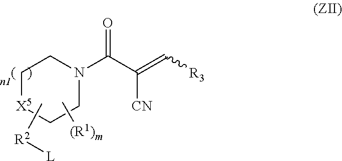
wherein R² is absent or is selected from the group consisting of aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
R^{y} is optionally substituted C₁₋₆alkyl or H;
R³ is selected from the group consisting of C₁ to C₆ alkyl, cycloalkyl, heterocycloalkyl, aryl, heteroaryl, substituted aryl, and substituted heteroaryl, optionally wherein the C₁ to C₆ alkyl is optionally substituted with a heterocycloalkyl group;
X⁵ is CR^{b}₂, NR^{b}, O or a 5- to 7-membered heterocycloalkyl;
each R¹ is independently selected from the group consisting of aryl, substituted aryl, heteroaryl, substituted heteroaryl, cycloalkyl, substituted cycloalkyl, heterocycloalkyl, substituted heterocycloalkyl, C₁ to C₆ alkyl and substituted C₁ to C₆ alkyl, and/or wherein two R¹ groups combine to form an optionally substituted C₁₋₃ bridge or optionally substituted C₃₋₅cycloalkyl;
each R^{b} is independently H or optionally substituted C₁₋₃alkyl;
n1 is 0, 1, 2 or 3;
m is 0, 1 or 2; and
L shows the point of attachment of the linker.

7. (canceled)

8. The bifunctional molecule of claim 1, wherein Z comprises a structure according to formula (ZIVa) to (ZIVj): 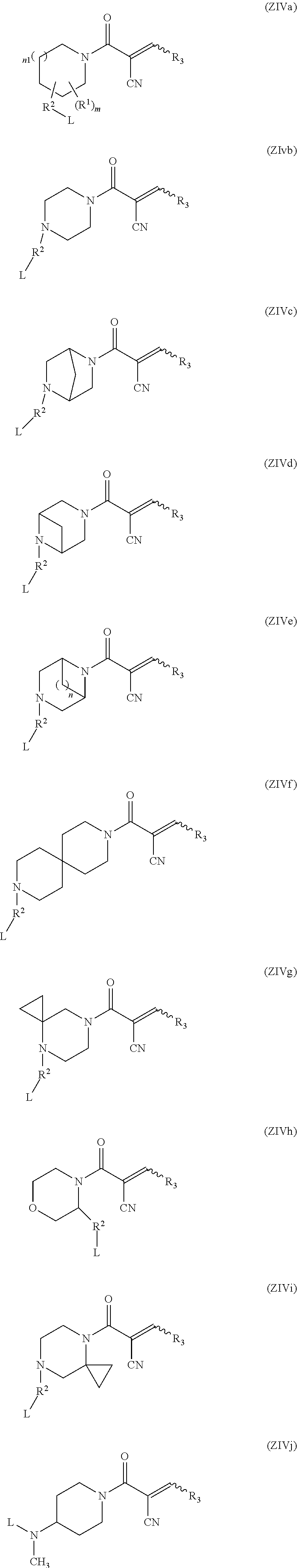
wherein R² is absent or is selected from the group consisting of aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
R^{y} is optionally substituted C₁₋₆alkyl or H;
R³ is selected from the group consisting of C₁ to C₆ alkyl, cycloalkyl, heterocycloalkyl, aryl, heteroaryl, substituted cycloalkyl, substituted heterocycloalkyl, substituted aryl, and substituted heteroaryl, wherein the C₁ to C₆ alkyl is optionally substituted with a heterocycloalkyl group;
each R¹ is independently selected from the group consisting of aryl, substituted aryl, heteroaryl, substituted heteroaryl, cycloalkyl, substituted cycloalkyl, C₁ to C₆ alkyl and substituted C₁ to C₆ alkyl;
n1 is 0, 1 or 2;
n′ is 1 or 2;
m is 0, 1 or 2; and
L shows the point of attachment of the linker.

9-23. (canceled)

24. The bifunctional molecule of claim 6, wherein R¹ is selected from one of the following structures:

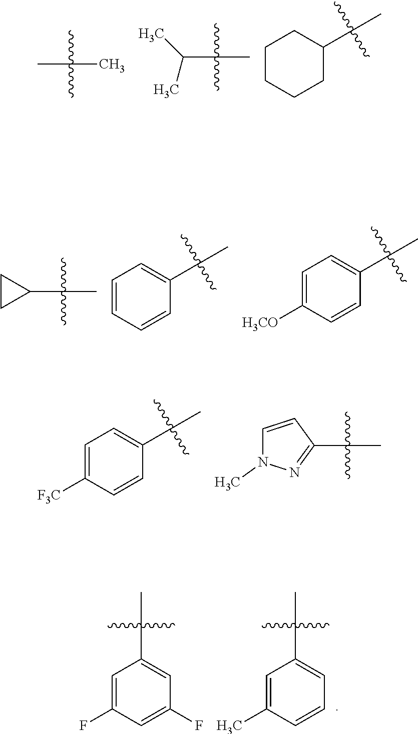

25. The bifunctional molecule of claim 1, wherein R² is) absent.

26. (canceled)

27. The bifunctional molecule of claim 1, wherein R³ is 0
selected from the group consisting of C₁ to C₆ alkyl optionally substituted with a heterocycloalkyl group having 5 to 7 ring atoms and containing 1 or 2 heteroatoms each independently selected from N, O and S; aryl having 6 to 10 carbon ring atoms; and heteroaryl having 5 to 10 ring atoms and containing 1 to 3 heteroatoms each independently selected from N, O and S; wherein the aryl and the heteroaryl are optionally substituted with one or two substituents selected from the group consisting of halo, C₁ to C₃ alkyl, C₁ to C₃ haloalkyl and C₁ to C₃ alkoxy.

28. (canceled)

29. The bifunctional molecule of claim 1, wherein R³ is selected from one of the following structures:

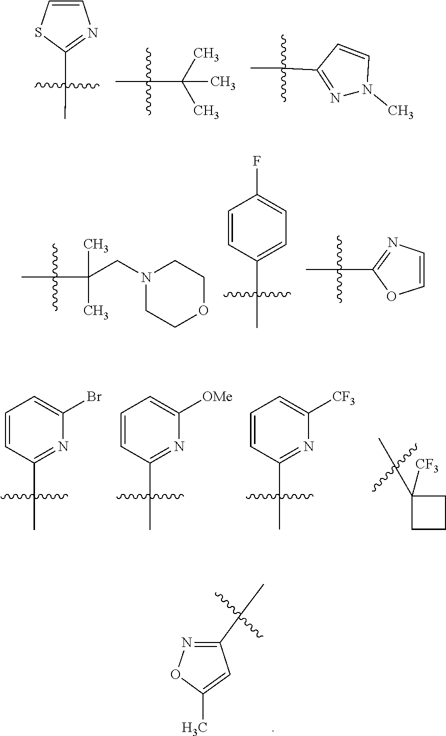

30. The bifunctional molecule of claim 1, wherein Z comprises one of the following structures: 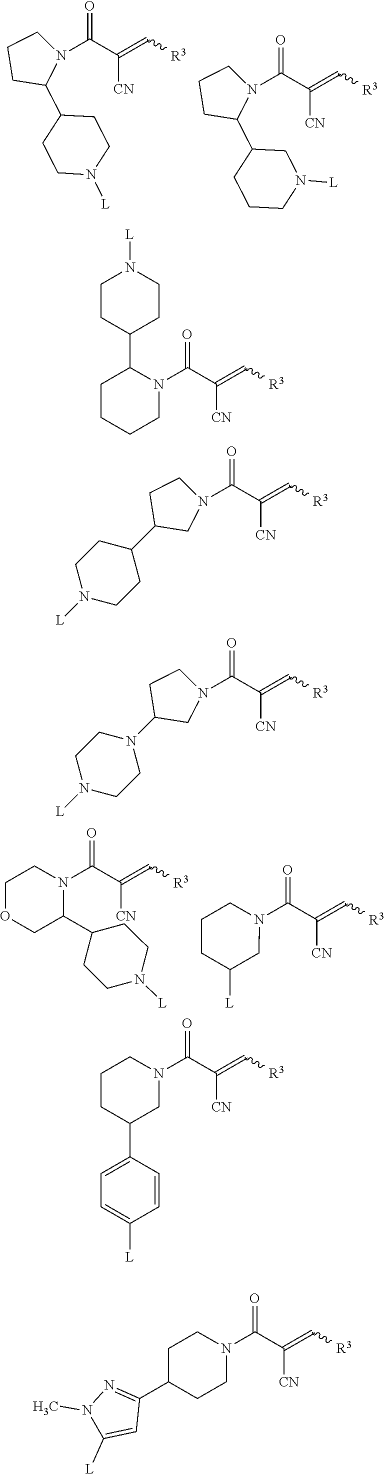 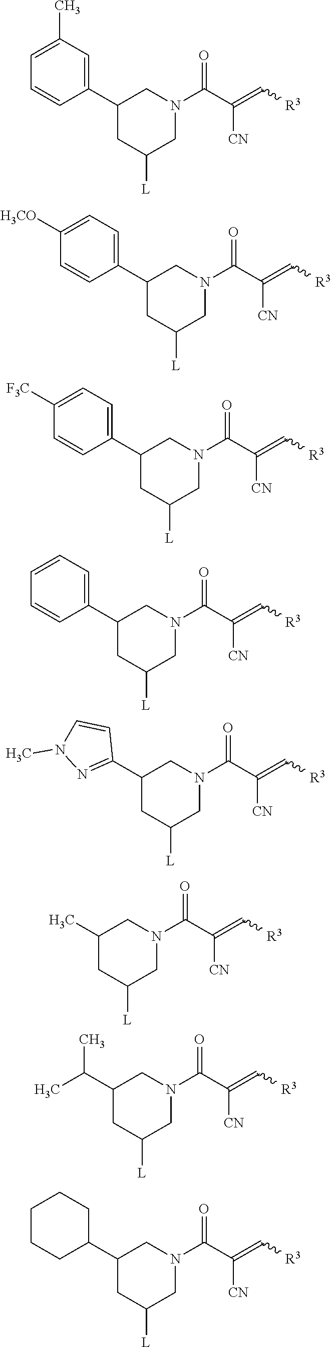 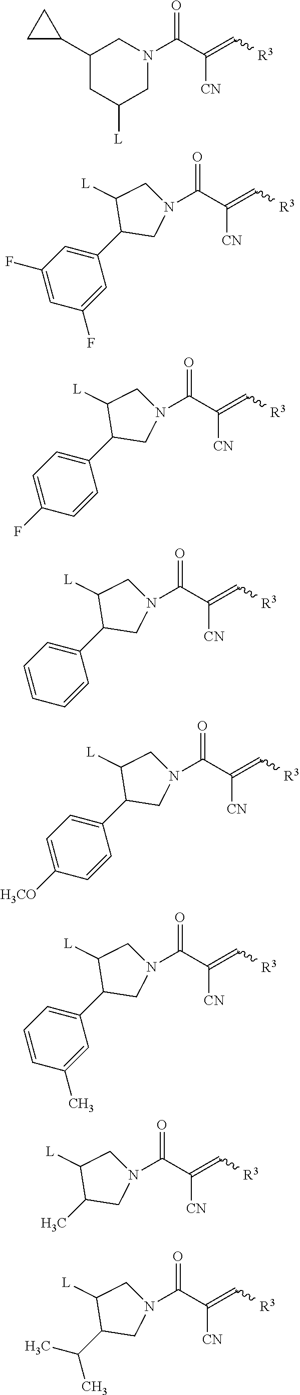 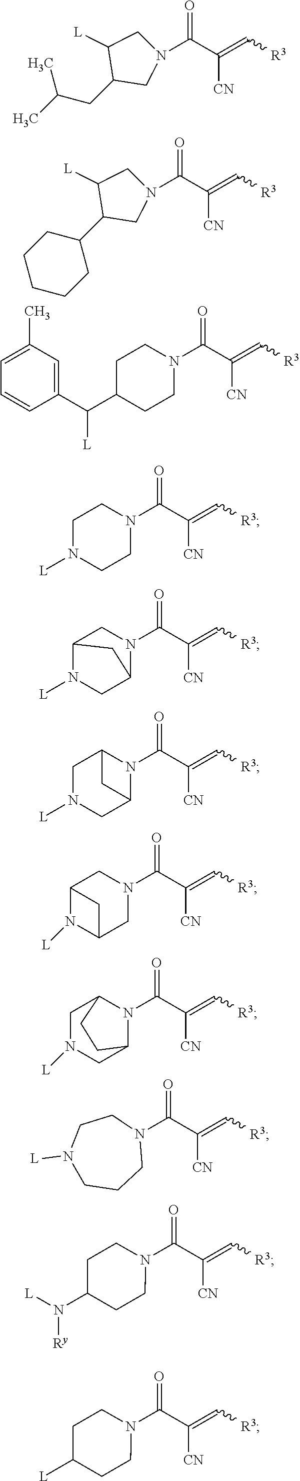 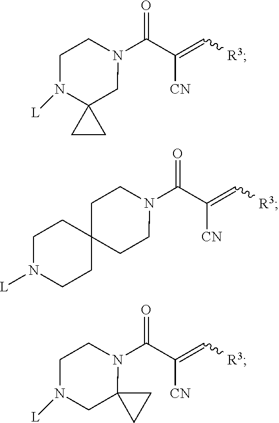
wherein R³ in each of the structures above is one of the following:

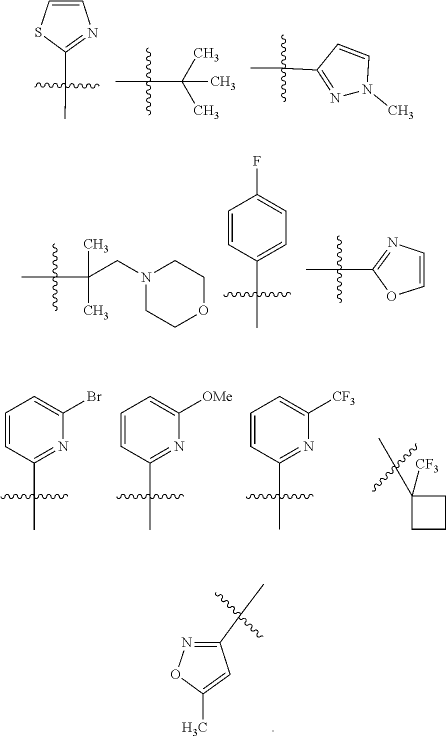

31-33. (canceled)

34. The bifunctional molecule according to claim 1, wherein the linker (L) has formula (L1a): 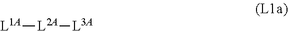
wherein L^{1A} is absent or is selected from the group consisting of C₁-C₆ alkylene, C₁-C₆ alkoxy and C₁-C₆ alkylamino;
L^{2A} is —NR^{L2A}C═O— or —C═ONR^{L2A}—; and
L^{3A} is selected from the group consisting of C₁-C₃ alkylene, C₁-C₆ alkoxy and C₁-C₆ alkylamino;
wherein R^{L2A} is H or C₁-C₆ alkyl;
or, the structure of the linker (L) has formula (L1b): 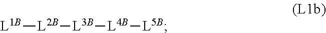
wherein L^{1B} is absent or is selected from the group consisting of C₁-C₃ alkylene, C₁-C₆ alkoxy and C₁-C₆ alkylamino;
L^{2B} is —NR^{L2A}C═O— or —C═ONR^{L2A}—;
L^{3B} is selected from the group consisting of C₁-C₁₅ alkylene and —[(CH₂)₂O]₁₋₆ (CH₂)₂—;
L^{4B} is —NR^{L2A}C═O— or —C═ONR^{L2A}— wherein R^{L2A} is H or C₁-C₆ alkyl;
L^{5B} is selected from the group consisting of C₁-C₃ alkylene, C₁-C₆ alkoxy and C₁-C₆ alkylamino;
wherein R^{L2A} is H or C₁-C₆ alkyl;
or, the structure of the linker (L) has formula (L1c): 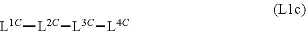
wherein L^{1C} is an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from the group consisting of N, O and S;
L^{2C} is absent or is selected from the group consisting of C₁-C₃ alkylene, C₁-C₆ alkoxy and C₁-C₆ alkylamino;
L^{3C} is —R^{L2B}C═O— or —(C═O)R^{L2B}—; and
L^{4C} is selected from the group consisting of C₁-C₃ alkylene, C₁-C₆ alkoxy and C₁-C₆ alkylamino;
wherein:
R^{L2A} is H or C₁-C₆ alkyl; and
R^{L2B} is NR^{L2A}, or an N-linked optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from the group consisting of N, O and S;
or, the structure of the linker (L) has formula (L1d): 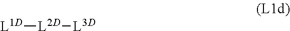
wherein L^{1D} is absent or is selected from the group consisting of C₁-C₃ alkylene, CO, and C₁-C₃ alkylene (N(C₁-C₃ alkyl);
L^{2D} is NR^{L2A} or an optionally substituted 4- to 7-membered monocyclic N-heterocycloalkyl, an optionally substituted 7- to 12-membered bicyclic N-heterocycloalkyl, or an optionally substituted 8- to 18-membered tricyclic N-heterocycloalkyl, each optionally containing one or two additional ring heteroatoms selected from the group consisting of N, O and S; wherein R^{L2A} is H or C₁-C₆ alkyl; and
L^{3D} is absent or is selected from the group consisting of C₁-C₃ alkylene, —O—, —N(C₁-C₃ alkyl)-, and CO;
or, the structure of the linker (L) has formula (L1e): 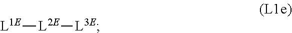
wherein L^{1E} is C₁-C₃ alkylene or CO;
L^{2E} is an optionally substituted 4- to 7-membered monocyclic N-heterocycloalkyl, an optionally substituted 7- to 12-membered bicyclic N-heterocycloalkyl, each optionally containing one or two additional ring heteroatoms selected from the group consisting of N, O and S; and
L^{3E} is selected from C₁-C₃ alkylene;
or, the linker (L) may be represented as shown in has formula (L1f): 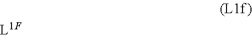
wherein L^{1F} is selected from C₁-C₃ alkylene, CO, and C₁-C₃ alkylene(NR^{L1C}); wherein R^{L1C} is H or C₁-C₃ alkyl.

35. The bifunctional molecule according to claim 1, wherein the target protein binding ligand (TBL) is selected from the group consisting of: (i) binders to kinases; (ii) binders to bromodomain-containing proteins; (iii) epigenetic modulator compounds; (iv) binders to transcription factors; (vi) binders to phosphatases, (vii) binders to ubiquitin E3 ligases and/or deubiquitinase enzymes; (viii) binders to nuclear hormone receptors; (ix) binders to aggregation-prone proteins; (x) binders to apoptotic & anti-apoptotic factors; and (xi) binders to polymerases.

36. A pharmaceutical composition comprising the bifunctional molecule according to claim 1, or a pharmaceutically acceptable salt thereof, and a pharmaceutically acceptable carrier.

37-39. (canceled)

40. A method of treating and/or preventing any disease or condition which is associated with and/or is caused by an abnormal level of protein activity, the method comprising administering a therapeutically effective amount of a bifunctional molecule of claim 1, or a pharmaceutically acceptable salt thereof, to a subject in need thereof.

41. The method of claim 40, wherein the disease or condition is cancer.

42. A method of selectively degrading and/or increasing proteolysis of a target protein in a cell, the method comprising contacting and/or treating the cell with a bifunctional molecule of claim 1, or a pharmaceutically acceptable salt thereof.

43. A method of selectively degrading and/or increasing proteolysis of a target protein in a subject in need thereof, the method comprising administering to the subject a therapeutically effective amount of a bifunctional molecule of claim 1, or a pharmaceutically acceptable salt thereof.

44-45. (canceled)

46. A compound according to formula (VI): 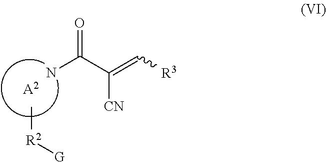
wherein;
ring A² is an optionally substituted 4- to 7-membered monocyclic N-heterocycloalkyl or an optionally substituted 7- to 12-membered bicyclic N-heterocycloalkyl, each optionally containing one or two additional ring heteroatoms selected from N, O and S;
R² is absent or is selected from the group consisting of aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
R^{y} is optionally substituted C₁₋₆alkyl or H;
R³ is selected from the group consisting of C₁₋₆ alkyl, cycloalkyl, substituted cycloalkyl, alkylcycloalkyl, substituted alkylcycloalkyl, alkyl heterocycloalkyl, substituted alkylcycloalkyl, heterocycloalkyl, substituted heterocycloalkyl, aryl, substituted aryl, heteroaryl, and substituted heteroaryl, wherein the C₁₋₆ alkyl is optionally substituted with one or more halo; and
G is configured to enable attachment of the Z moiety to another chemical structure via formation of a new covalent bond.

47. A compound comprising the structure having Formula (ZI): 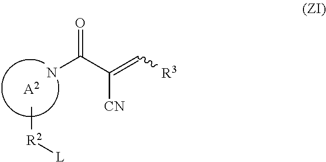
wherein:
ring A² is an optionally substituted 4- to 7-membered monocyclic N-heterocycloalkyl or an optionally substituted 7- to 12-membered bicyclic N-heterocycloalkyl, each optionally containing one or two additional ring heteroatoms selected from N, O and S;
R² is absent or is selected from the group consisting of aryl, substituted aryl, heteroaryl, substituted heteroaryl, heterocycloalkyl, substituted heterocycloalkyl, NR^{y}, —CH(aryl)-, —CH(substituted aryl)-, —CH(heteroaryl)- and —CH(substituted heteroaryl)-;
R^{y} is optionally substituted C₁₋₆alkyl or H;
R³ is selected from the group consisting of C₁₋₆ alkyl, cycloalkyl, substituted cycloalkyl, alkylcycloalkyl, substituted alkylcycloalkyl, alkyl heterocycloalkyl, substituted alkylcycloalkyl, heterocycloalkyl, substituted heterocycloalkyl, aryl, substituted aryl, heteroaryl, and substituted heteroaryl, wherein the C₁₋₆ alkyl is optionally substituted with one or more halo; and
L is a linker.

48. (canceled)

49. A method of making the bifunctional molecule of claim 1.

50. A method of screening the bifunctional molecules according to claim 1, comprising:
a. providing a bifunctional molecule comprising:
(i) a first ligand comprising a structure according to Z as defined in claim 1;
(ii) a second ligand that binds to a target protein; and
(iii) a linker that covalently attaches the first and second ligands;
b. contacting a cell with the bifunctional molecule;
c. detecting degradation of the target protein in the cell;
d. detecting degradation of the target protein in the cell in the absence of the bifunctional molecule; and
e. comparing the level of degradation of the target protein in the cell contacted with the bifunctional molecule to the level of degradation of the target protein in the absence of the bifunctional molecule;
wherein an increased level of degradation of the target protein in the cell contacted with the bifunctional molecule indicates that the bifunctional molecule has facilitated and/or promoted the degradation of the target protein,
optionally wherein detecting degradation of the target protein comprises detecting changes in the levels of the target protein in the cell.

51-52. (canceled)

53. The bifunctional molecule of claim 1 selected from the group consisting of: 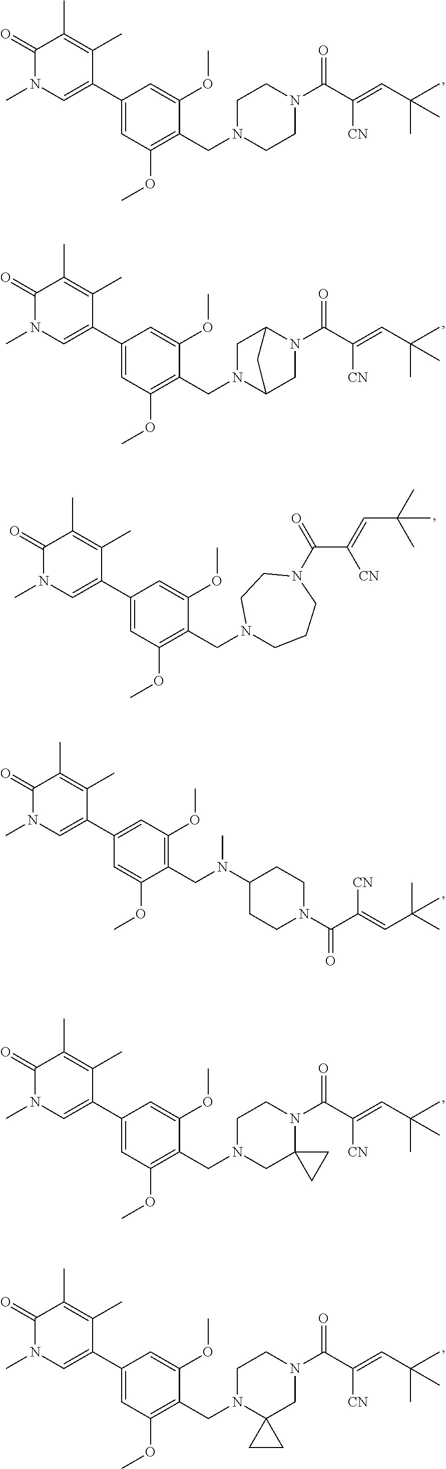 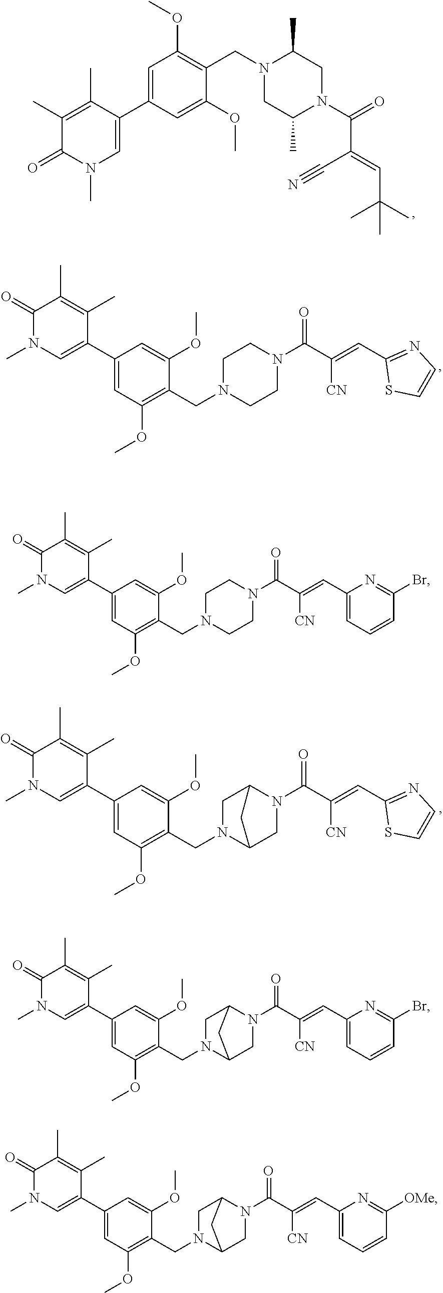 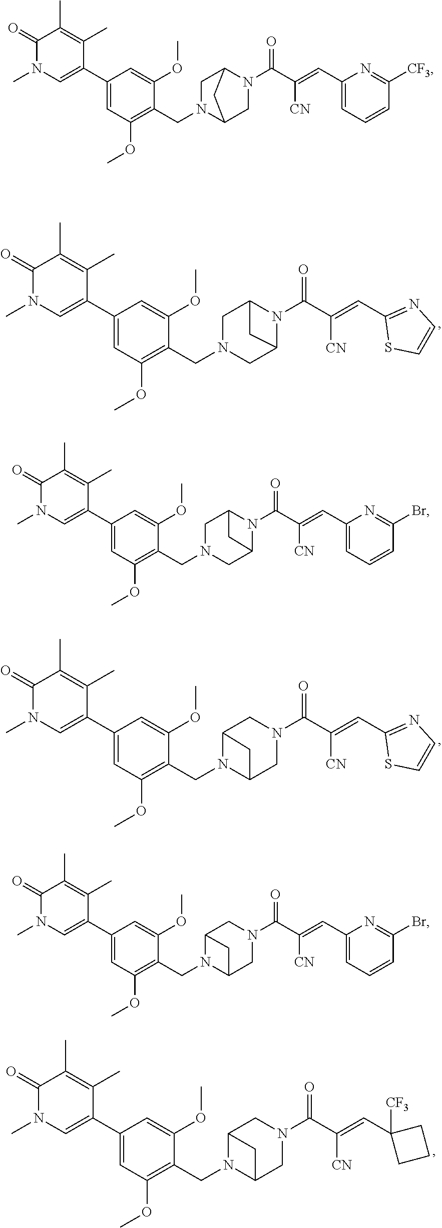 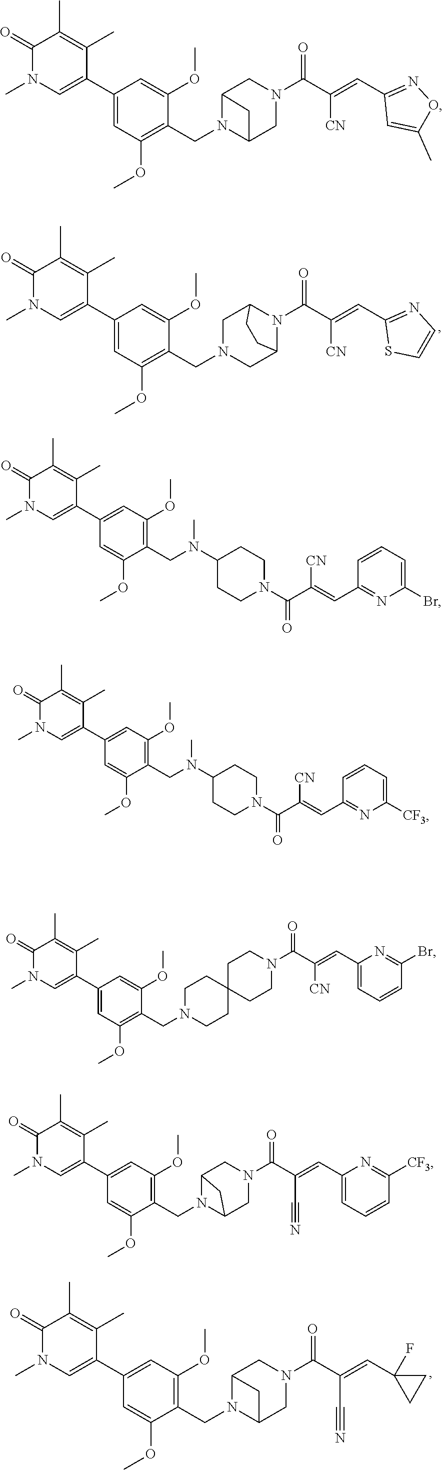 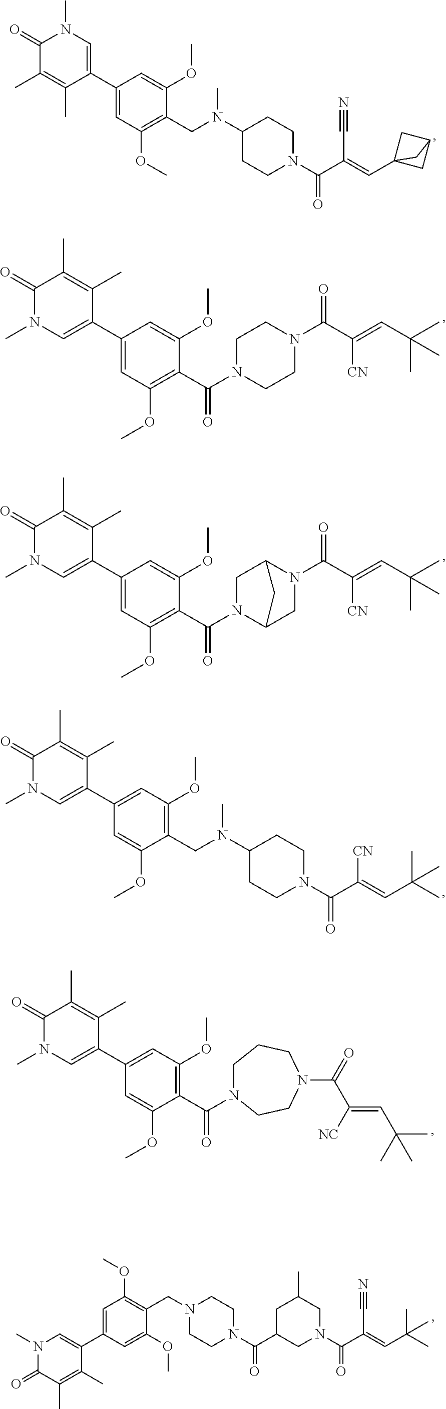 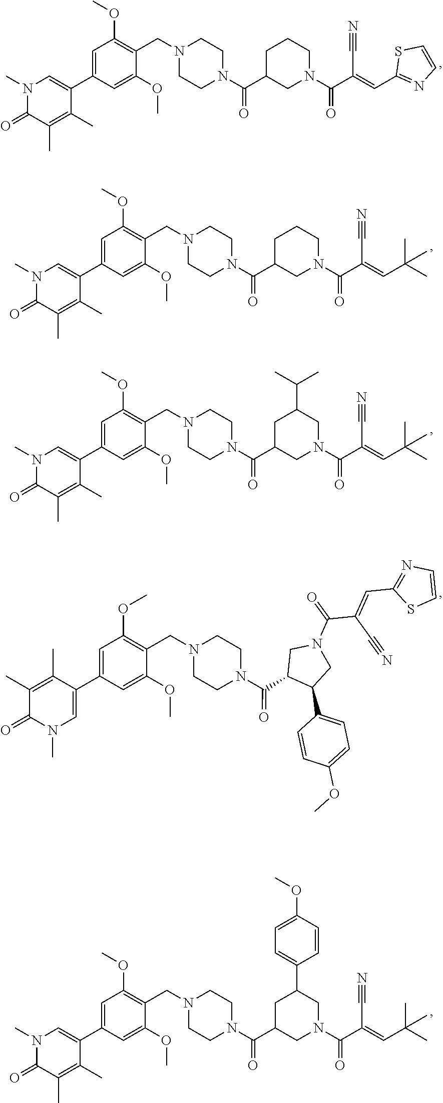 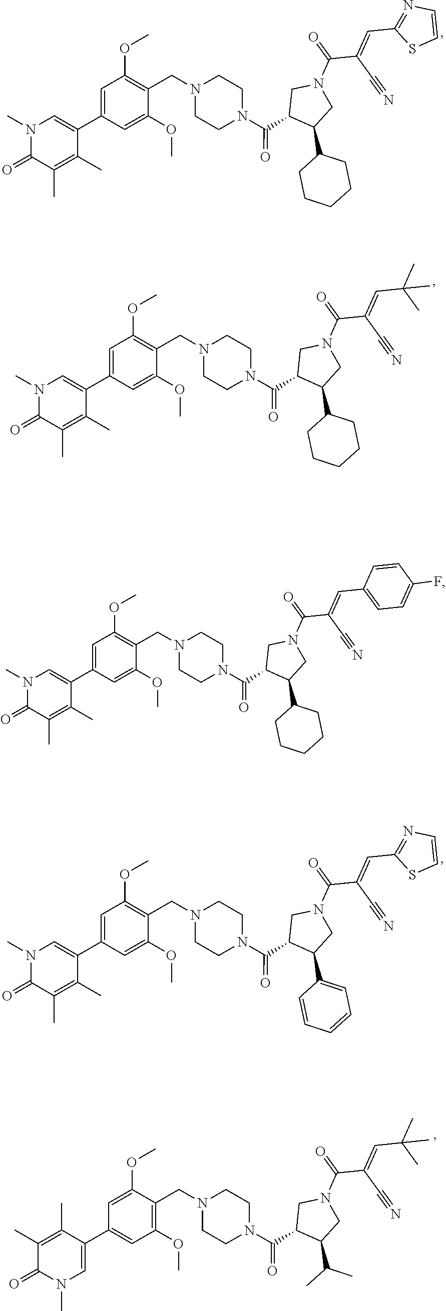 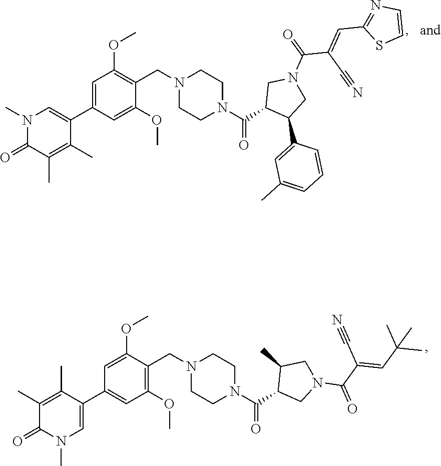 [SMILES: COc1cc(-c2cn(C)c(=O)c(C)c2C)cc(OC)c1CN1CCN(C(=O)[C@@H]2CN(C(=O)/C(C#N)=C/c3nccs3)C[C@H]2c2cccc(C)c2)CC1 ; "," ; "and" ; COc1cc(-c2cn(C)c(=O)c(C)c2C)cc(OC)c1CN1CCN(C(=O)[C@H]2CN(C(=O)/C(C#N)=C/C(C)(C)C)C[C@@H]2C)CC1 ; ","]
or a pharmaceutically acceptable salt thereof.
